SEQUENCE LISTING (1041 sequences)

SEQ ID NO: 1 - protein, 446 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, synthetic construct
DYGMH

SEQ ID NO: 4 - protein, 17 aa, synthetic construct
YISSGSSTIYYADTVKG

SEQ ID NO: 5 - protein, 7 aa, synthetic construct
RGLLLDY

SEQ ID NO: 6 - protein, 19 aa, synthetic construct
MGWSCIILFLVATATGVHS

SEQ ID NO: 7 - protein, 218 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, synthetic construct
RASKSVSTSSYSYMH

SEQ ID NO: 10 - protein, 7 aa, synthetic construct
YASYLES

SEQ ID NO: 11 - protein, 9 aa, synthetic construct
QHSREFPWT

SEQ ID NO: 12 - protein, 19 aa, synthetic construct
MGWSCIILFLVATATGVHS

SEQ ID NO: 13 - protein, 446 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, synthetic construct
NYGMH

SEQ ID NO: 16 - protein, 5 aa, synthetic construct
BYGMH

SEQ ID NO: 17 - protein, 17 aa, synthetic construct
YISSGSSTIYYADTVKG

SEQ ID NO: 18 - protein, 7 aa, synthetic construct
RGLLLDY

SEQ ID NO: 19 - protein, 19 aa, synthetic construct
MGWSCIILFLVATATGVHS

SEQ ID NO: 20 - protein, 218 aa, synthetic construct; identical to SEQ ID NO: 7

SEQ ID NO: 21 - protein, 111 aa, synthetic construct; identical to SEQ ID NO: 8

SEQ ID NO: 22 - protein, 15 aa, synthetic construct
RASKSVSTSSYSYMH

SEQ ID NO: 23 - protein, 7 aa, synthetic construct
YASYLES

SEQ ID NO: 24 - protein, 9 aa, synthetic construct
QHSREFPWT

SEQ ID NO: 25 - protein, 19 aa, synthetic construct
MGWSCIILFLVATATGVHS

SEQ ID NO: 26 - protein, 17 aa, synthetic construct
YISSGSSTIYYADSVKG

SEQ ID NO: 27 - protein, 17 aa, synthetic construct
YISSSSSTIYYADSVKG

SEQ ID NO: 28 - protein, 15 aa, synthetic construct
RASKSVSTSSYSYLA

SEQ ID NO: 29 - protein, 15 aa, synthetic construct
RASKTVSTSSYSYMH

SEQ ID NO: 30 - protein, 7 aa, synthetic construct
YASYLQS

SEQ ID NO: 31 - protein, 17 aa, synthetic construct
  VARIANT 5: note = X is separately any amino acid
  VARIANT 14: note = X is separately any amino acid
YISSXSSTIYYADXVKG

SEQ ID NO: 32 - protein, 15 aa, synthetic construct
  VARIANT 5: note = X is separately any amino acid
  VARIANT 14..15: note = X are separately any amino acids
RASKXVSTSSYSYXX

SEQ ID NO: 33 - protein, 7 aa, synthetic construct
  VARIANT 6: note = X can be any amino acid
YASYLXS

SEQ ID NO: 34 - protein, 5 aa, synthetic construct
QYGMH

SEQ ID NO: 35 - protein, 5 aa, synthetic construct
EYGMH

SEQ ID NO: 36 - skipped

SEQ ID NO: 37 - protein, 5 aa, synthetic construct
  VARIANT 5: note = X can be K or R
QYGMX

SEQ ID NO: 38 - protein, 5 aa, synthetic construct
  VARIANT 5: note = X can be K or R
EYGMX

SEQ ID NO: 39 - protein, 17 aa, synthetic construct
YISSGSSTIYYAETVKG

SEQ ID NO: 40 - protein, 17 aa, synthetic construct
  VARIANT 16: note = X can be R or H
YISSGSSTIYYADTVXG

SEQ ID NO: 41 - protein, 17 aa, synthetic construct
  VARIANT 16: note = X can be R or H
YISSGSSTIYYAETVXG

SEQ ID NO: 42 - protein, 7 aa, synthetic construct
  VARIANT 1: note = X can be K or H
XGLLLDY

SEQ ID NO: 43 - protein, 8 aa, synthetic construct
RGLLLBEY

SEQ ID NO: 44 - protein, 7 aa, synthetic construct
  VARIANT 1: note = X can be K or H
XGLLLEY

SEQ ID NO: 45 - protein, 15 aa, synthetic construct
  VARIANT 1: note = X can be K or H
XASKSVSTSSYSYMH

SEQ ID NO: 46 - protein, 15 aa, synthetic construct
  VARIANT 4: note = X can be R or H
RASXSVSTSSYSYMH

SEQ ID NO: 47 - protein, 15 aa, synthetic construct
  VARIANT 15: note = X can be K or R
RASKSVSTSSYSYMX

SEQ ID NO: 48 - protein, 15 aa, synthetic construct
  VARIANT 1: note = X can be K or H
  VARIANT 4: note = X can be R or H
XASXSVSTSSYSYMH

SEQ ID NO: 49 - protein, 15 aa, synthetic construct
  VARIANT 1: note = X can be K or H
  VARIANT 15: note = X can be K or R
XASKSVSTSSYSYMX

SEQ ID NO: 50 - protein, 15 aa, synthetic construct
  VARIANT 15: note = X can be K or R
  VARIANT 4: note = X can be R or H
RASXSVSTSSYSYMX

SEQ ID NO: 51 - protein, 7 aa, synthetic construct
YASYLDS

SEQ ID NO: 52 - protein, 9 aa, synthetic construct
  VARIANT 2: note = X can be K or R
QXSREFPWT

SEQ ID NO: 53 - protein, 9 aa, synthetic construct
  VARIANT 4: note = X can be K or H
QHSXEFPWT

SEQ ID NO: 54 - protein, 9 aa, synthetic construct
QHSRDFPWT

SEQ ID NO: 55 - protein, 9 aa, synthetic construct
  VARIANT 2: note = X can be K or R
  VARIANT 4: note = X can be K or H
QXSXEFPWT

SEQ ID NO: 56 - protein, 9 aa, synthetic construct
  VARIANT 2: note = X can be K or R
QXSRDFPWT

SEQ ID NO: 57 - protein, 9 aa, synthetic construct
  VARIANT 4: note = X can be K or H
QHSXDFPWT

SEQ ID NO: 58 - skipped

SEQ ID NO: 59 - protein, 17 aa, synthetic construct
  VARIANT 5: note = X can be G or S
  VARIANT 14: note = X can be T or S
  VARIANT 16: note = X can be K, R, or H
YISSXSSTIYYABXVXG

SEQ ID NO: 60 - protein, 7 aa, synthetic construct
  VARIANT 1: note = X can be K, R, or H
XGLLLBY

SEQ ID NO: 61 - protein, 15 aa, synthetic construct
  VARIANT 1: note = X can be K, R, or H
  VARIANT 4: note = X can be K, R, or H
  VARIANT 5: note = X can be T or S
  VARIANT 14: note = X can be M or L
  VARIANT 15: note = X can be K, R, H, or A
XASXXVSTSSYSYXX

SEQ ID NO: 62 - protein, 7 aa, synthetic construct
  VARIANT 6: note = X cab be D, E, N, or Q
YASYLXS

SEQ ID NO: 63 - protein, 9 aa, synthetic construct
  VARIANT 2: note = X can be K, R, or H
  VARIANT 4: note = X can be K, R, or H
QXSXBFPWT

SEQ ID NO: 64 - protein, 116 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSS

SEQ ID NO: 65 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSS

SEQ ID NO: 66 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSS

SEQ ID NO: 67 - protein, 116 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSS

SEQ ID NO: 68 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 69 - protein, 116 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 70 - protein, 116 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSS

SEQ ID NO: 71 - protein, 446 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 446 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 446 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 446 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 465 aa, synthetic construct
MGWSCIILFLVATATGVHSEVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 465 aa, synthetic construct
MGWSCIILFLVATATGVHSEVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 465 aa, synthetic construct
MGWSCIILFLVATATGVHSEVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 465 aa, synthetic construct
MGWSCIILFLVATATGVHSEVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIK

SEQ ID NO: 86 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIK

SEQ ID NO: 87 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIK

SEQ ID NO: 88 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIK

SEQ ID NO: 89 - protein, 111 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIK

SEQ ID NO: 90 - protein, 111 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIK

SEQ ID NO: 91 - protein, 218 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 218 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 218 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 237 aa, synthetic construct
MGWSCIILFLVATATGVHSDIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 237 aa, synthetic construct
MGWSCIILFLVATATGVHSDIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 116 aa, synthetic construct
EVQLVESGGGLVQPGGSLRLSCSASGFTFSNYGMHWVRQAPGKGLEYVSYISSGSSTIYYADTVKGRFTISRDNSKNTLYLQMSSLRAEDTAVYYCVKRGLLLDYWGQGTLVTVSS

SEQ ID NO: 104 - protein, 116 aa, synthetic construct
EVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 105 - protein, 116 aa, synthetic construct
EVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 106 - protein, 116 aa, synthetic construct
EVQLVQSGGGLIQPGGSLRLSCAASGFTFSNYGMHWVRQAPGKGLEWVSYISSGSSTIYYADSVKGRFTISRDNSKNTLYLQMNSLRAEDTAVYYCARRGLLLDYWGQGTTVTVSS

SEQ ID NO: 107 - protein, 116 aa, synthetic construct
EVQLVESGGGLIQPGGSLRLSCAASGFTFSNYGMHWVRQAPGKGLEWVSYISSGSSTIYYADSVKGRFTISRDNSKNTLYLQMTSLRAEDTAVYYCARRGLLLDYWGQGTTLTVSS

SEQ ID NO: 108 - protein, 116 aa, synthetic construct
EVQLQESGGGVVQPGGSLRLSCAASGFTFSNYGMHWIRQAPGKGLEWVSYISSGSSTIYYADSVKGRFTISRDNSKNTLYLQMNSLRSEDTAVYYCARRGLLLDYWGQGTLVTVSS

SEQ ID NO: 109 - protein, 116 aa, synthetic construct
EVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 110 - protein, 116 aa, synthetic construct
EVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 111 - protein, 116 aa, synthetic construct
EVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 112 - protein, 116 aa, synthetic construct
EVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 113 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGQGTKVEIK

SEQ ID NO: 114 - protein, 111 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIK

SEQ ID NO: 115 - protein, 111 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIK

SEQ ID NO: 116 - protein, 111 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIK

SEQ ID NO: 117 - protein, 111 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 118 - protein, 111 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIK

SEQ ID NO: 119 - protein, 111 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIK

SEQ ID NO: 120 - protein, 111 aa, synthetic construct
DIQMTQSPSSLSASVGDRVTITCRASKSVSTSSYSYLAWYQQKPEKAPKLLIKYASYLQSGVPSRFSGSGSGTDFTLTISSLQPEDFATYYCQHSREFPWTFGAGTKLELK

SEQ ID NO: 121 - protein, 111 aa, synthetic construct
DIQMTQSPSSLSASVGDRVTISCRASKSVSTSSYSYMHWYQQKPEKAPKLLIKYASYLQSGVPSRFSGSGSGTDFTLTISSLQPEDVATYYCQHSREFPWTFGAGTKLELK

SEQ ID NO: 122 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 123 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 124 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSSSSTIYYADSVKGRFTISRDNAKNSLYLQMNSLRAEDTAVYYCARRGLLLDYWGQGTTVTVSS

SEQ ID NO: 125 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 126 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 127 - protein, 515 aa, synthetic construct
DIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKSVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 128 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 129 - protein, 515 aa, synthetic construct
DIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 130 - protein, 515 aa, synthetic construct
DIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKSVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSSSSTIYYADSVKGRFTISRDNSKNTLYLQMNSLRAEDTAVYYCARRGLLLDYWGQGTTVTVSS

SEQ ID NO: 131 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 132 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 133 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 134 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGDVKPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 135 - protein, 515 aa, synthetic construct
DIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQAPKLLIKYASYLESGVPSRFSGSGSGTDFTLTISSLQPEDFATYYCQHSREFPWTFGQGTKVEIKRADAAPGGGGSGGGGSGGGGSEVQLVESGGGLVQPGGSLRLSCAASGFTFSNYGMHWVRQAPGKGLEWVSYISSGSSTIYYADSVKGRFTISRDNAKNSLYLQMNSLRAEDTAVYYCARRGLLLDYWGQGTTVTVSS

SEQ ID NO: 136 - protein, 515 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 137 - protein, 515 aa, synthetic construct
DIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSSASTKGPSVFPLAPLESSGSDIQMTQSPSSLSASVGDRVTITCRASKTVSTSSYSYMHWYQQKPGKAPKLLIKYASYLESGVPSRFSGSGSGTDFTLTISSLQPEDFATYYCQHSREFPWTFGQGTKVEIKRADAAPGGGGSGGGGSGGGGSEVQLVESGGGVVQPGGSLRLSCAASGFTFSNYGMHWVRQAPEKGLEWVSYISSGSSTIYYADSVKGRFTISRDNSKNTLYLQMNSLRAEDTAVYYCARRGLLLDYWGQGTTVTVSS

SEQ ID NO: 138 - RNA, 25 nt, synthetic construct
  source 1..25: mol_type = other RNA
gcttttttgccctgcgtgaccagat

SEQ ID NO: 139 - RNA, 21 nt, synthetic construct
  source 1..21: mol_type = other RNA
gccacagcatacatcctgttt

SEQ ID NO: 140 - RNA, 21 nt, synthetic construct
  source 1..21: mol_type = other RNA
gccacagcatacatcctgttt

SEQ ID NO: 141 - RNA, 27 nt, synthetic construct
  source 1..27: mol_type = other RNA
atctggtcacgcagggcaaaaaagctc

SEQ ID NO: 142 - RNA, 21 nt, synthetic construct
  source 1..21: mol_type = other RNA
acaggatgtatgctgtggctt

SEQ ID NO: 143 - RNA, 21 nt, synthetic construct
  source 1..21: mol_type = other RNA
acaggatgtatgctgtggctt

SEQ ID NO: 144 - DNA, 25 bp, synthetic construct
  misc_feature 24..25: note = DNA
  misc_feature 1..23: note = RNA
  source 1..25: mol_type = other DNA
  modified_base order(2,5,7,11,12,14,17,18): mod_base = OTHER; note = 2-Prime-O-methyl-modified nucleotides
  modified_base 1..25: mod_base = OTHER; note = Phosphodiester linkages
gcttttttgccctgcgtgaccagat

SEQ ID NO: 145 - RNA, 21 nt, synthetic construct
  source 1..21: mol_type = other RNA
  modified_base 1..21: mod_base = OTHER; note = Phosphodiester linkages
gccacagcatacatcctgttt

SEQ ID NO: 146 - RNA, 21 nt, synthetic construct
  source 1..21: mol_type = other RNA
  modified_base order(2,4,6,8,10,12,14,16,18,20): mod_base = OTHER; note = 2-Prime-O-methyl-modified nucleotides
  modified_base order(1,3,5,7,9,11,13,15,17,19,21): mod_base = OTHER; note = 2-Prime-fluoro-modified nucleotides
  modified_base 1..3: mod_base = OTHER; note = Phosphorothioate linkages
  modified_base 3..21: mod_base = OTHER; note = Phosphodiester linkages
gccacagcatacatcctgttt

SEQ ID NO: 147 - RNA, 27 nt, synthetic construct
  source 1..27: mol_type = other RNA
  modified_base order(1..4,11,13,23,25..27): mod_base = OTHER; note = 2-Prime-O-methyl-modified nucleotides
  modified_base 1..27: mod_base = OTHER; note = Phosphodiester linkages
atctggtcacgcagggcaaaaaagctc

SEQ ID NO: 148 - RNA, 21 nt, synthetic construct
  source 1..21: mol_type = other RNA
  modified_base 1..21: mod_base = OTHER; note = Phosphodiester linkages
acaggatgtatgctgtggctt

SEQ ID NO: 149 - RNA, 21 nt, synthetic construct
  source 1..21: mol_type = other RNA
  modified_base order(1,3,5,7,9,11,13,15,17,19,21): mod_base = OTHER; note = 2-Prime-O-methyl-modified nucleotides
  modified_base order(2,4,6,8,10,12,14,16,18,20): mod_base = OTHER; note = 2-Prime-fluoro-modified nucleotides
  modified_base order(1..3,19..21): mod_base = OTHER; note = Phosphorothioate linkages
  modified_base 3..19: mod_base = OTHER; note = Phosphodiester linkages
acaggatgtatgctgtggctt

SEQ ID NO: 150 - DNA, 13 bp, synthetic construct
  source 1..13: mol_type = other DNA
  misc_feature order(1..7,9,11..13): note = RNA
  misc_feature order(8,10): note = DNA
ttcaccatctctc

SEQ ID NO: 151 - DNA, 13 bp, synthetic construct
  misc_feature 4..11: note = DNA
  misc_feature order(1..3,12,13): note = RNA
  source 1..13: mol_type = other DNA
  modified_base order(1..3,12,13): mod_base = OTHER; note = 2-Prime-O-methyl-modified ribose nucleotides
  modified_base 1..13: mod_base = OTHER; note = Phosphorothioate linkages
ttcaccatctctc

SEQ ID NO: 152 - RNA, 23 nt, synthetic construct
  source 1..23: mol_type = other RNA
gtaaccaagagtattccatdtdt

SEQ ID NO: 153 - RNA, 23 nt, synthetic construct
  source 1..23: mol_type = other RNA
atggaatactcttggttacdtdt

SEQ ID NO: 154 - RNA, 23 nt, synthetic construct
  source 1..23: mol_type = other RNA
tcttggttacatgaaatcccatc

SEQ ID NO: 155 - RNA, 21 nt, synthetic construct
  source 1..21: mol_type = other RNA
tgggatttcatgtaaccaaga

SEQ ID NO: 156 - RNA, 21 nt, synthetic construct
  source 1..21: mol_type = other RNA
cagaaagagtgtctcatctta

SEQ ID NO: 157 - RNA, 23 nt, synthetic construct
  source 1..23: mol_type = other RNA
aatgatgagacactctttctggt

SEQ ID NO: 158 - RNA, 23 nt, synthetic construct
  source 1..23: mol_type = other RNA
tatatttccaggatgaaagtcca

SEQ ID NO: 159 - RNA, 21 nt, synthetic construct
  source 1..21: mol_type = other RNA
gactttcatcctggaaatata

SEQ ID NO: 160 - RNA, 23 nt, synthetic construct
  source 1..23: mol_type = other RNA
acaaaagcaaaacaggtctagaa

SEQ ID NO: 161 - RNA, 22 nt, synthetic construct
  source 1..22: mol_type = other RNA
ctagacctgttttgcttttgtn

SEQ ID NO: 162 - RNA, 36 nt, synthetic construct
  source 1..36: mol_type = other RNA
atgttgtcctttttatctgagcagccgaaaggctgc

SEQ ID NO: 163 - RNA, 22 nt, synthetic construct
  source 1..22: mol_type = other RNA
tcagataaaaaggacaacatgg

SEQ ID NO: 164 - RNA, 21 nt, synthetic construct
  source 1..21: mol_type = other RNA
ggttaacaccatttacttcaa

SEQ ID NO: 165 - RNA, 23 nt, synthetic construct
  source 1..23: mol_type = other RNA
ttgaagtaaatggtgttaaccag

SEQ ID NO: 166 - RNA, 19 nt, synthetic construct
  source 1..19: mol_type = other RNA
gagaatatttcacccttca

SEQ ID NO: 167 - RNA, 19 nt, synthetic construct
  source 1..19: mol_type = other RNA
tgaagggtgaaatattctc

SEQ ID NO: 168 - RNA, 19 nt, synthetic construct
  source 1..19: mol_type = other RNA
aggagttccacattctggc

SEQ ID NO: 169 - RNA, 19 nt, synthetic construct
  source 1..19: mol_type = other RNA
gccagaatgtggaactcct

SEQ ID NO: 170 - RNA, 19 nt, synthetic construct
  source 1..19: mol_type = other RNA
tgaagtagaagatgcgctt

SEQ ID NO: 171 - RNA, 19 nt, synthetic construct
  source 1..19: mol_type = other RNA
aagcgcatcttctacttca

SEQ ID NO: 172 - RNA, 36 nt, synthetic construct
  source 1..36: mol_type = other RNA
aaaccctttgtcttcttaaagcagccgaaaggctgc

SEQ ID NO: 173 - RNA, 22 nt, synthetic construct
  source 1..22: mol_type = other RNA
tttaagaagacaaagggtttgg

SEQ ID NO: 174 - RNA, 21 nt, synthetic construct
  source 1..21: mol_type = other RNA
ctggatcacatgcacaatgtt

SEQ ID NO: 175 - RNA, 21 nt, synthetic construct
  source 1..21: mol_type = other RNA
cattgtgcatgtgatccagtt

SEQ ID NO: 176 - RNA, 22 nt, synthetic construct
  source 1..22: mol_type = other RNA
aagcaagatatttttataatan

SEQ ID NO: 177 - RNA, 25 nt, synthetic construct
  source 1..25: mol_type = other RNA
tattataaaaatatcttgctttttt

SEQ ID NO: 178 - protein, 31 aa, synthetic construct
RASKSVSTSSYSYMHWYQQKPGQPPKLLIKY

SEQ ID NO: 179 - protein, 31 aa, synthetic construct
RASKTVSTSSYSYMHWYQQKPGQPPKLLIKY

SEQ ID NO: 180 - protein, 33 aa, synthetic construct
RVTITCRASKSVSTSSYSYMHWYQQKPGKAPKL

SEQ ID NO: 181 - protein, 32 aa, synthetic construct
  VARIANT 1: note = X is any residue, such as a basic residue (R or K)
  VARIANT 30: note = X is any residue, such as a basic residue (R or K)
XRASKTVSTSSYSYMHWYQQKPGQPPKLLXKY

SEQ ID NO: 182 - protein, 7 aa, synthetic construct
GQSSRSS

SEQ ID NO: 183 - protein, 18 aa, synthetic construct
GQSSRSSSGGGSSGGGGS

SEQ ID NO: 184 - protein, 4 aa, synthetic construct
GSGS

SEQ ID NO: 185 - protein, 4 aa, synthetic construct
GGGS

SEQ ID NO: 186 - protein, 10 aa, synthetic construct
GGGGSGGGGS

SEQ ID NO: 187 - protein, 20 aa, synthetic construct
GGGGSGGGGSGGGGSGGGGS

SEQ ID NO: 188 - protein, 15 aa, synthetic construct
GGGGSGGGGSGGGGS

SEQ ID NO: 189 - protein, 21 aa, synthetic construct
RADAAPGGGGSGGGGSGGGGS

SEQ ID NO: 190 - protein, 19 aa, synthetic construct
ASTKGPSVFPLAPLESSGS

SEQ ID NO: 191 - protein, 8 aa, synthetic construct
GFTFSDYG

SEQ ID NO: 192 - protein, 8 aa, synthetic construct
GFTFSNYG

SEQ ID NO: 193 - protein, 8 aa, synthetic construct
ISSGSSTI

SEQ ID NO: 194 - protein, 8 aa, synthetic construct
ISSSSSTI

SEQ ID NO: 195 - protein, 9 aa, synthetic construct
ARRGLLLDY

SEQ ID NO: 196 - protein, 10 aa, synthetic construct
KSVSTSSYSY

SEQ ID NO: 197 - protein, 10 aa, synthetic construct
KTVSTSSYSY

SEQ ID NO: 198 - protein, 9 aa, synthetic construct
QHSREFPWT

SEQ ID NO: 199 - protein, 108 aa, synthetic construct
MTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKVEIK

SEQ ID NO: 200 - protein, 116 aa, synthetic construct; identical to SEQ ID NO: 105

SEQ ID NO: 201 - RNA, 25 nt, synthetic construct
  source 1..25: mol_type = other RNA
tagctgcaaagcaagtgaagcggac

SEQ ID NO: 202 - RNA, 25 nt, synthetic construct
  source 1..25: mol_type = other RNA
atcacaaacacaacctacctcaggc

SEQ ID NO: 203 - RNA, 20 nt, synthetic construct
  source 1..20: mol_type = other RNA
tcgcaaacagtacaatggcg

SEQ ID NO: 204 - RNA, 15 nt, synthetic construct
  source 1..15: mol_type = other RNA
aacagtacaatggcg

SEQ ID NO: 205 - RNA, 15 nt, synthetic construct
  source 1..15: mol_type = other RNA
tcgcaaacagtacaa

SEQ ID NO: 206 - RNA, 15 nt, synthetic construct
  source 1..15: mol_type = other RNA
actagtcgcaaacag

SEQ ID NO: 207 - RNA, 15 nt, synthetic construct
  source 1..15: mol_type = other RNA
agctaactagtcgca

SEQ ID NO: 208 - RNA, 28 nt, synthetic construct
  source 1..28: mol_type = other RNA
agcccatcttctcctggtcctgggaagg

SEQ ID NO: 209 - RNA, 26 nt, synthetic construct
  source 1..26: mol_type = other RNA
gtgtctttctgagccgccatttctca

SEQ ID NO: 210 - RNA, 22 nt, synthetic construct
  source 1..22: mol_type = other RNA
tctgttagccacgccgccattt

SEQ ID NO: 211 - RNA, 24 nt, synthetic construct
  source 1..24: mol_type = other RNA
ctgttagccactcgccgccatttc

SEQ ID NO: 212 - RNA, 24 nt, synthetic construct
  source 1..24: mol_type = other RNA
ttgtgtctttcttctgttagccac

SEQ ID NO: 213 - RNA, 26 nt, synthetic construct
  source 1..26: mol_type = other RNA
tgttagccactgatctcaacagatct

SEQ ID NO: 214 - RNA, 26 nt, synthetic construct
  source 1..26: mol_type = other RNA
gttcagcttctgttctcaacagatct

SEQ ID NO: 215 - RNA, 26 nt, synthetic construct
  source 1..26: mol_type = other RNA
ttgtgtctttctgcgccgccatttct

SEQ ID NO: 216 - RNA, 26 nt, synthetic construct
  source 1..26: mol_type = other RNA
ctgagaaactgttcgccgccatttct

SEQ ID NO: 217 - RNA, 26 nt, synthetic construct
  source 1..26: mol_type = other RNA
tctcaggaatttgcgccgccatttct

SEQ ID NO: 218 - RNA, 26 nt, synthetic construct
  source 1..26: mol_type = other RNA
tgttagccactgacgccgccatttct

SEQ ID NO: 219 - RNA, 26 nt, synthetic construct
  source 1..26: mol_type = other RNA
gttcagcttctgtcgccgccatttct

SEQ ID NO: 220 - RNA, 30 nt, synthetic construct
  source 1..30: mol_type = other RNA
attctcaggaatttgaacgccgccatttct

SEQ ID NO: 221 - RNA, 30 nt, synthetic construct
  source 1..30: mol_type = other RNA
atttgtgtctttctgtttctcaacagatct

SEQ ID NO: 222 - RNA, 25 nt, synthetic construct
  source 1..25: mol_type = other RNA
attctcaggaatttgtttctcaaca

SEQ ID NO: 223 - RNA, 30 nt, synthetic construct
  source 1..30: mol_type = other RNA
atttgtgtctttctgaacgccgccatttct

SEQ ID NO: 224 - RNA, 26 nt, synthetic construct
  source 1..26: mol_type = other RNA
tctcaggaatttgtctcaacagatct

SEQ ID NO: 225 - RNA, 26 nt, synthetic construct
  source 1..26: mol_type = other RNA
gtttgccgctgcctcctggagttcct

SEQ ID NO: 226 - RNA, 24 nt, synthetic construct
  source 1..24: mol_type = other RNA
gtttgccgctgccctggagttcct

SEQ ID NO: 227 - RNA, 30 nt, synthetic construct
  source 1..30: mol_type = other RNA
cagtttgccgctgcccatcctggagttcct

SEQ ID NO: 228 - RNA, 26 nt, synthetic construct
  source 1..26: mol_type = other RNA
cagtttgccgctgccctggagttcct

SEQ ID NO: 229 - RNA, 30 nt, synthetic construct
  source 1..30: mol_type = other RNA
ttcttccccagttgcgccatcctggagttc

SEQ ID NO: 230 - RNA, 30 nt, synthetic construct
  source 1..30: mol_type = other RNA
cagacctcctgccacgccatcctggagttc

SEQ ID NO: 231 - RNA, 26 nt, synthetic construct
  source 1..26: mol_type = other RNA
ttgccgctgcccacatcctggagttc

SEQ ID NO: 232 - RNA, 26 nt, synthetic construct
  source 1..26: mol_type = other RNA
gccgctgcccacatcctggagttcct

SEQ ID NO: 233 - RNA, 26 nt, synthetic construct
  source 1..26: mol_type = other RNA
ccgctgcccaatgtcctggagttcct

SEQ ID NO: 234 - RNA, 25 nt, synthetic construct
  source 1..25: mol_type = other RNA
gctgcccaatgccatcctggagttc

SEQ ID NO: 235 - RNA, 25 nt, synthetic construct
  source 1..25: mol_type = other RNA
gctgcccaatgccatcctggagttc

SEQ ID NO: 236 - RNA, 25 nt, synthetic construct
  source 1..25: mol_type = other RNA
ttgccgctgcccaatgccatcctgg

SEQ ID NO: 237 - RNA, 25 nt, synthetic construct
  source 1..25: mol_type = other RNA
acagtttgccgctgcccaatgccat

SEQ ID NO: 238 - RNA, 25 nt, synthetic construct
  source 1..25: mol_type = other RNA
tgacaacagtttgccgctgcccaat

SEQ ID NO: 239 - RNA, 25 nt, synthetic construct
  source 1..25: mol_type = other RNA
tgttctgacaacagtttgccgctgc

SEQ ID NO: 240 - RNA, 25 nt, synthetic construct
  source 1..25: mol_type = other RNA
ttcaatgttctgacaacagtttgcc

SEQ ID NO: 241 - RNA, 25 nt, synthetic construct
  source 1..25: mol_type = other RNA
ttgcattcaatgttctgacaacagt

SEQ ID NO: 242 - RNA, 25 nt, synthetic construct
  source 1..25: mol_type = other RNA
cccagttgcattcaatgttctgaca

SEQ ID NO: 243 - RNA, 26 nt, synthetic construct
  source 1..26: mol_type = other RNA
ccaatgccatcctggagttcctgtaa

SEQ ID NO: 244 - RNA, 22 nt, synthetic construct
  source 1..22: mol_type = other RNA
caatgccatcctggagttcctg

SEQ ID NO: 245 - RNA, 34 nt, synthetic construct
  source 1..34: mol_type = other RNA
gcccaatgccatcctggagttcctgtaagatacc

SEQ ID NO: 246 - RNA, 31 nt, synthetic construct
  source 1..31: mol_type = other RNA
gcccaatgccatcctggagttcctgtaagat

SEQ ID NO: 247 - RNA, 39 nt, synthetic construct
  source 1..39: mol_type = other RNA
ttgccgctgcccaatgccatcctggagttcctgtaagat

SEQ ID NO: 248 - RNA, 22 nt, synthetic construct
  source 1..22: mol_type = other RNA
caatgccatcctggagttcctg

SEQ ID NO: 249 - RNA, 21 nt, synthetic construct
  source 1..21: mol_type = other RNA
atgggatccagtatacttaca

SEQ ID NO: 250 - RNA, 20 nt, synthetic construct
  source 1..20: mol_type = other RNA
atgggatccagtatacttac

SEQ ID NO: 251 - RNA, 19 nt, synthetic construct
  source 1..19: mol_type = other RNA
tgggatccagtatacttac

SEQ ID NO: 252 - RNA, 19 nt, synthetic construct
  source 1..19: mol_type = other RNA
atgggatccagtatactta

SEQ ID NO: 253 - RNA, 19 nt, synthetic construct
  source 1..19: mol_type = other RNA
gatccagtatacttacagg

SEQ ID NO: 254 - RNA, 19 nt, synthetic construct
  source 1..19: mol_type = other RNA
aatgggatccagtatactt

SEQ ID NO: 255 - RNA, 30 nt, synthetic construct
  source 1..30: mol_type = other RNA
ctccaacatcaaggaagatggcatttctag

SEQ ID NO: 256 - RNA, 20 nt, synthetic construct
  source 1..20: mol_type = other RNA
tcaaggaagatggcatttct

SEQ ID NO: 257 - RNA, 30 nt, synthetic construct
  source 1..30: mol_type = other RNA
gtgtcaccagagtaacagtctgagtaggag

SEQ ID NO: 258 - RNA, 30 nt, synthetic construct
  source 1..30: mol_type = other RNA
aggttgtgtcaccagagtaacagtctgagt

SEQ ID NO: 259 - RNA, 30 nt, synthetic construct
  source 1..30: mol_type = other RNA
ggcagtttccttagtaaccacaggttgtgt

SEQ ID NO: 260 - RNA, 30 nt, synthetic construct
  source 1..30: mol_type = other RNA
agatggcagtttccttagtaaccacaggtt

SEQ ID NO: 261 - RNA, 30 nt, synthetic construct
  source 1..30: mol_type = other RNA
atggcatttctagtttggagatggcagttt

SEQ ID NO: 262 - RNA, 30 nt, synthetic construct; identical to SEQ ID NO: 255
  source 1..30: mol_type = other RNA

SEQ ID NO: 263 - RNA, 30 nt, synthetic construct; identical to SEQ ID NO: 255
  source 1..30: mol_type = other RNA

SEQ ID NO: 264 - RNA, 21 nt, synthetic construct
  source 1..21: mol_type = other RNA
cctccggttctgaaggtgttc

SEQ ID NO: 265 - RNA, 30 nt, synthetic construct
  source 1..30: mol_type = other RNA
attcaactgttgcctccggttctgaaggtg

SEQ ID NO: 266 - RNA, 25 nt, synthetic construct
  source 1..25: mol_type = other RNA
ctgaaggtgttcttgtacttcatcc

SEQ ID NO: 267 - RNA, 31 nt, synthetic construct
  source 1..31: mol_type = other RNA
cattcaactgttgcctccggttctgaaggtg

SEQ ID NO: 268 - RNA, 21 nt, synthetic construct
  source 1..21: mol_type = other RNA
cctccggttctgaaggtgttc

SEQ ID NO: 269 - RNA, 25 nt, synthetic construct
  source 1..25: mol_type = other RNA
gttgcctccggttctgaaggtgttc

SEQ ID NO: 270 - RNA, 25 nt, synthetic construct
  source 1..25: mol_type = other RNA
gttgcctccggttctgaaggtgttc

SEQ ID NO: 271 - RNA, 21 nt, synthetic construct
  source 1..21: mol_type = other RNA
cctccggttctgaaggtgttc

SEQ ID NO: 272 - RNA, 25 nt, synthetic construct
  source 1..25: mol_type = other RNA
gttgcctccggttctgaaggtgttc

SEQ ID NO: 273 - RNA, 21 nt, synthetic construct
  source 1..21: mol_type = other RNA
gcagcctctcgctcactcacc

SEQ ID NO: 274 - RNA, 21 nt, synthetic construct
  source 1..21: mol_type = other RNA
ccaaagcagcctctcgctcac

SEQ ID NO: 275 - RNA, 21 nt, synthetic construct
  source 1..21: mol_type = other RNA
ttcttccaaagcagcctctcg

SEQ ID NO: 276 - RNA, 21 nt, synthetic construct
  source 1..21: mol_type = other RNA
atctatgagtttcttccaaag

SEQ ID NO: 277 - RNA, 21 nt, synthetic construct
  source 1..21: mol_type = other RNA
tgttgcagtaatctatgagtt

SEQ ID NO: 278 - RNA, 21 nt, synthetic construct
  source 1..21: mol_type = other RNA
cagggggaactgttgcagtaa

SEQ ID NO: 279 - RNA, 21 nt, synthetic construct
  source 1..21: mol_type = other RNA
tttccaggtccagggggaact

SEQ ID NO: 280 - RNA, 21 nt, synthetic construct
  source 1..21: mol_type = other RNA
gcaagaaacttttccaggtcc

SEQ ID NO: 281 - skipped

SEQ ID NO: 282 - RNA, 12 nt, synthetic construct
  source 1..12: mol_type = other RNA
cagcagcagcag

SEQ ID NO: 283 - RNA, 15 nt, synthetic construct
  source 1..15: mol_type = other RNA
cagcagcagcagcag

SEQ ID NO: 284 - RNA, 18 nt, synthetic construct
  source 1..18: mol_type = other RNA
cagcagcagcagcagcag

SEQ ID NO: 285 - skipped

SEQ ID NO: 286 - RNA, 12 nt, synthetic construct
  source 1..12: mol_type = other RNA
caggcaggcagg

SEQ ID NO: 287 - skipped

SEQ ID NO: 288 - RNA, 12 nt, synthetic construct
  source 1..12: mol_type = other RNA
aggcaggcaggc

SEQ ID NO: 289 - skipped

SEQ ID NO: 290 - RNA, 12 nt, synthetic construct
  source 1..12: mol_type = other RNA
ggcaggcaggca

SEQ ID NO: 291 - RNA, 18 nt, synthetic construct
  source 1..18: mol_type = other RNA
tcactttcataatgctgg

SEQ ID NO: 292 - RNA, 25 nt, synthetic construct
  source 1..25: mol_type = other RNA
ctgccaaggagggccctgctcagcc

SEQ ID NO: 293 - RNA, 25 nt, synthetic construct
  source 1..25: mol_type = other RNA
cgcactcttgcccacaccgccggcg

SEQ ID NO: 294 - RNA, 25 nt, synthetic construct
  source 1..25: mol_type = other RNA
ctcacctctatagtggggtcgtatt

SEQ ID NO: 295 - RNA, 21 nt, synthetic construct
  source 1..21: mol_type = other RNA
ttgcccacaccgccggcgccc

SEQ ID NO: 296 - RNA, 21 nt, synthetic construct
  source 1..21: mol_type = other RNA
ttgcccacaccgacggcgccc

SEQ ID NO: 297 - RNA, 25 nt, synthetic construct
  source 1..25: mol_type = other RNA
tatagtggggtcgtattcgtccaca

SEQ ID NO: 298 - RNA, 25 nt, synthetic construct
  source 1..25: mol_type = other RNA
tggttctggatcagctggatggtca

SEQ ID NO: 299 - RNA, 25 nt, synthetic construct
  source 1..25: mol_type = other RNA
cggggtcctcctacagggtctcctg

SEQ ID NO: 300 - RNA, 25 nt, synthetic construct
  source 1..25: mol_type = other RNA
gtcatcgctcctcaggggcctgcgg

SEQ ID NO: 301 - RNA, 25 nt, synthetic construct
  source 1..25: mol_type = other RNA
cccaccaccaccagcttatattccg

SEQ ID NO: 302 - RNA, 25 nt, synthetic construct
  source 1..25: mol_type = other RNA
gggtctcctgccccacctgccaagg

SEQ ID NO: 303 - RNA, 25 nt, synthetic construct
  source 1..25: mol_type = other RNA
ttcagtcattttcagcaggccttat

SEQ ID NO: 304 - RNA, 25 nt, synthetic construct
  source 1..25: mol_type = other RNA
tttacctctattgttggatcatatt

SEQ ID NO: 305 - RNA, 25 nt, synthetic construct
  source 1..25: mol_type = other RNA
atgaaaatggtcagagaaaccttta

SEQ ID NO: 306 - RNA, 25 nt, synthetic construct
  source 1..25: mol_type = other RNA
tctgtatcaaagaatggtcctgcac

SEQ ID NO: 307 - RNA, 25 nt, synthetic construct
  source 1..25: mol_type = other RNA
cagtaatatgcatattaaaacaaga

SEQ ID NO: 308 - RNA, 25 nt, synthetic construct
  source 1..25: mol_type = other RNA
aataaaaataatgaaaatgtgacta

SEQ ID NO: 309 - RNA, 25 nt, synthetic construct
  source 1..25: mol_type = other RNA
tattagaacatgtcacacataaggt

SEQ ID NO: 310 - RNA, 25 nt, synthetic construct
  source 1..25: mol_type = other RNA
agtttatattcagtcattttcagca

SEQ ID NO: 311 - RNA, 25 nt, synthetic construct
  source 1..25: mol_type = other RNA
tacgccaccagctccaactaccaca

SEQ ID NO: 312 - RNA, 25 nt, synthetic construct
  source 1..25: mol_type = other RNA
gctgtatcgtcaaggcactcttgcc

SEQ ID NO: 313 - RNA, 25 nt, synthetic construct
  source 1..25: mol_type = other RNA
tcgtccacaaaatgattctgaatta

SEQ ID NO: 314 - RNA, 25 nt, synthetic construct
  source 1..25: mol_type = other RNA
tttcacaccagcaagaacctgttgg

SEQ ID NO: 315 - RNA, 25 nt, synthetic construct
  source 1..25: mol_type = other RNA
cctcacctctatggtgggatcatat

SEQ ID NO: 316 - RNA, 25 nt, synthetic construct
  source 1..25: mol_type = other RNA
accaccagtttgtactcagtcattt

SEQ ID NO: 317 - RNA, 25 nt, synthetic construct
  source 1..25: mol_type = other RNA
caacaccacctgctccaaccaccac

SEQ ID NO: 318 - RNA, 25 nt, synthetic construct
  source 1..25: mol_type = other RNA
gtgcgcttttcccaacaccacctgc

SEQ ID NO: 319 - RNA, 25 nt, synthetic construct
  source 1..25: mol_type = other RNA
tggttctggattagctggattgtca

SEQ ID NO: 320 - RNA, 25 nt, synthetic construct
  source 1..25: mol_type = other RNA
ggtgggatcatattcatctacaaag

SEQ ID NO: 321 - RNA, 25 nt, synthetic construct
  source 1..25: mol_type = other RNA
aatcagggttaattggcgagccaca

SEQ ID NO: 322 - RNA, 25 nt, synthetic construct
  source 1..25: mol_type = other RNA
caggatcaggtcagcgggctaccac

SEQ ID NO: 323 - RNA, 25 nt, synthetic construct
  source 1..25: mol_type = other RNA
aaaaatgtctcctgcgagagcaaac

SEQ ID NO: 324 - RNA, 25 nt, synthetic construct
  source 1..25: mol_type = other RNA
ccaccatggacaagaaaaatgtctc

SEQ ID NO: 325 - RNA, 22 nt, synthetic construct
  source 1..22: mol_type = other RNA
cagcacgcatcccaccatggac

SEQ ID NO: 326 - RNA, 25 nt, synthetic construct
  source 1..25: mol_type = other RNA
atcactgtgtctggagcaacaatgc

SEQ ID NO: 327 - RNA, 25 nt, synthetic construct
  source 1..25: mol_type = other RNA
tcgtttgataggctccccttcgcag

SEQ ID NO: 328 - RNA, 25 nt, synthetic construct
  source 1..25: mol_type = other RNA
atcagggagaaaactcacctctcgt

SEQ ID NO: 329 - RNA, 22 nt, synthetic construct
  source 1..22: mol_type = other RNA
acctacccagttcggaatgatc

SEQ ID NO: 330 - RNA, 18 nt, synthetic construct
  source 1..18: mol_type = other RNA
ctacccagttcggaatga

SEQ ID NO: 331 - RNA, 15 nt, synthetic construct
  source 1..15: mol_type = other RNA
tacccagttcggaat

SEQ ID NO: 332 - RNA, 22 nt, synthetic construct
  source 1..22: mol_type = other RNA
atgtctcctctgttgtctaatg

SEQ ID NO: 333 - RNA, 22 nt, synthetic construct
  source 1..22: mol_type = other RNA
taacagcaaagacgatcattcc

SEQ ID NO: 334 - RNA, 20 nt, synthetic construct
  source 1..20: mol_type = other RNA
gggctctgggctcctgagcc

SEQ ID NO: 335 - RNA, 20 nt, synthetic construct
  source 1..20: mol_type = other RNA
ccgagctgctgcagtgggag

SEQ ID NO: 336 - RNA, 20 nt, synthetic construct
  source 1..20: mol_type = other RNA
gcaggtcccgcacagcacgg

SEQ ID NO: 337 - RNA, 20 nt, synthetic construct
  source 1..20: mol_type = other RNA
tggcagatgccttgtcggca

SEQ ID NO: 338 - RNA, 20 nt, synthetic construct
  source 1..20: mol_type = other RNA
gagccgctggcagatgcctt

SEQ ID NO: 339 - RNA, 20 nt, synthetic construct
  source 1..20: mol_type = other RNA
ctgagccgctggcagatgcc

SEQ ID NO: 340 - RNA, 20 nt, synthetic construct
  source 1..20: mol_type = other RNA
aggagatgggtccgcccacc

SEQ ID NO: 341 - RNA, 20 nt, synthetic construct
  source 1..20: mol_type = other RNA
cagaggagatgggtccgccc

SEQ ID NO: 342 - RNA, 20 nt, synthetic construct
  source 1..20: mol_type = other RNA
gccagaggagatgggtccgc

SEQ ID NO: 343 - RNA, 18 nt, synthetic construct
  source 1..18: mol_type = other RNA
aggtcccgggagaaggga

SEQ ID NO: 344 - RNA, 18 nt, synthetic construct
  source 1..18: mol_type = other RNA
cagccaggtcccgggaga

SEQ ID NO: 345 - RNA, 18 nt, synthetic construct
  source 1..18: mol_type = other RNA
ggagacagccaggtcccg

SEQ ID NO: 346 - RNA, 18 nt, synthetic construct
  source 1..18: mol_type = other RNA
agtctggagacagccagg

SEQ ID NO: 347 - RNA, 18 nt, synthetic construct
  source 1..18: mol_type = other RNA
gagcaagtctggagacag

SEQ ID NO: 348 - RNA, 18 nt, synthetic construct
  source 1..18: mol_type = other RNA
aaaaggagcaagtctgga

SEQ ID NO: 349 - RNA, 18 nt, synthetic construct
  source 1..18: mol_type = other RNA
gggacaaaaggagcaagt

SEQ ID NO: 350 - RNA, 18 nt, synthetic construct
  source 1..18: mol_type = other RNA
gcagtggtagtaagtggg

SEQ ID NO: 351 - RNA, 18 nt, synthetic construct
  source 1..18: mol_type = other RNA
gggatcagatcacagcca

SEQ ID NO: 352 - RNA, 18 nt, synthetic construct
  source 1..18: mol_type = other RNA
cagatcacagccatgcac

SEQ ID NO: 353 - RNA, 18 nt, synthetic construct
  source 1..18: mol_type = other RNA
cccagcgtcgccctgggc

SEQ ID NO: 354 - RNA, 18 nt, synthetic construct
  source 1..18: mol_type = other RNA
cccatcccagcgtcgccc

SEQ ID NO: 355 - RNA, 18 nt, synthetic construct
  source 1..18: mol_type = other RNA
cgtcacccatcccagcgt

SEQ ID NO: 356 - RNA, 18 nt, synthetic construct
  source 1..18: mol_type = other RNA
cctgacgtcacccatccc

SEQ ID NO: 357 - RNA, 18 nt, synthetic construct
  source 1..18: mol_type = other RNA
ggcagcctgacgtcaccc

SEQ ID NO: 358 - RNA, 18 nt, synthetic construct
  source 1..18: mol_type = other RNA
cagtgggcagcctgacgt

SEQ ID NO: 359 - RNA, 18 nt, synthetic construct
  source 1..18: mol_type = other RNA
acagtcagtgggcagcct

SEQ ID NO: 360 - RNA, 18 nt, synthetic construct
  source 1..18: mol_type = other RNA
acaggacagtcagtgggc

SEQ ID NO: 361 - RNA, 18 nt, synthetic construct
  source 1..18: mol_type = other RNA
acaggatcagcagagcct

SEQ ID NO: 362 - RNA, 18 nt, synthetic construct
  source 1..18: mol_type = other RNA
atcagcagagcctgccag

SEQ ID NO: 363 - RNA, 21 nt, synthetic construct
  source 1..21: mol_type = other RNA
agggcttaaaatttctcctgc

SEQ ID NO: 364 - RNA, 21 nt, synthetic construct
  source 1..21: mol_type = other RNA
ccaagggcttaaaatttctcc

SEQ ID NO: 365 - RNA, 21 nt, synthetic construct
  source 1..21: mol_type = other RNA
tttgaatcagagtccaagggc

SEQ ID NO: 366 - RNA, 21 nt, synthetic construct
  source 1..21: mol_type = other RNA
aagtttctcagtccacttggg

SEQ ID NO: 367 - RNA, 21 nt, synthetic construct
  source 1..21: mol_type = other RNA
ccggccattctctttctgttc

SEQ ID NO: 368 - RNA, 20 nt, synthetic construct
  source 1..20: mol_type = other RNA
atgcccggccattctctttc

SEQ ID NO: 369 - RNA, 20 nt, synthetic construct
  source 1..20: mol_type = other RNA
tgaccactgtagtagcaatg

SEQ ID NO: 370 - RNA, 20 nt, synthetic construct
  source 1..20: mol_type = other RNA
acaccattggttctcatagg

SEQ ID NO: 371 - RNA, 20 nt, synthetic construct
  source 1..20: mol_type = other RNA
atggtttgcagccacaacac

SEQ ID NO: 372 - RNA, 20 nt, synthetic construct
  source 1..20: mol_type = other RNA
ccacaacaccaatgcgatat

SEQ ID NO: 373 - RNA, 20 nt, synthetic construct
  source 1..20: mol_type = other RNA
gcatggtttgcagccacaac

SEQ ID NO: 374 - RNA, 20 nt, synthetic construct
  source 1..20: mol_type = other RNA
cttggggaagattctaaggt

SEQ ID NO: 375 - RNA, 18 nt, synthetic construct
  source 1..18: mol_type = other RNA
gtatccgctgagggaata

SEQ ID NO: 376 - RNA, 18 nt, synthetic construct
  source 1..18: mol_type = other RNA
gtgtagtatccgctgagg

SEQ ID NO: 377 - RNA, 18 nt, synthetic construct
  source 1..18: mol_type = other RNA
gatgggtgtagtatccgc

SEQ ID NO: 378 - RNA, 18 nt, synthetic construct
  source 1..18: mol_type = other RNA
aaatggatgggtgtagta

SEQ ID NO: 379 - RNA, 18 nt, synthetic construct
  source 1..18: mol_type = other RNA
ccttgaaatggatgggtg

SEQ ID NO: 380 - RNA, 18 nt, synthetic construct
  source 1..18: mol_type = other RNA
ggcgcagaaccaccaggt

SEQ ID NO: 381 - RNA, 18 nt, synthetic construct
  source 1..18: mol_type = other RNA
agggagttgcaatatcga

SEQ ID NO: 382 - RNA, 18 nt, synthetic construct
  source 1..18: mol_type = other RNA
gcctccacagattcaggg

SEQ ID NO: 383 - RNA, 18 nt, synthetic construct
  source 1..18: mol_type = other RNA
aaacttcattctggaaca

SEQ ID NO: 384 - RNA, 18 nt, synthetic construct
  source 1..18: mol_type = other RNA
agggctttggccattttc

SEQ ID NO: 385 - RNA, 18 nt, synthetic construct
  source 1..18: mol_type = other RNA
acagtttgcacttccggg

SEQ ID NO: 386 - RNA, 18 nt, synthetic construct
  source 1..18: mol_type = other RNA
cgcctaaggaattcactc

SEQ ID NO: 387 - RNA, 18 nt, synthetic construct
  source 1..18: mol_type = other RNA
cgcctaaggaattcactc

SEQ ID NO: 388 - RNA, 18 nt, synthetic construct
  source 1..18: mol_type = other RNA
ggatacgcctaaggaatt

SEQ ID NO: 389 - RNA, 18 nt, synthetic construct
  source 1..18: mol_type = other RNA
gccagtgcccatagcggg

SEQ ID NO: 390 - RNA, 18 nt, synthetic construct
  source 1..18: mol_type = other RNA
cttatgccagtgcccata

SEQ ID NO: 391 - RNA, 18 nt, synthetic construct
  source 1..18: mol_type = other RNA
cccaagggagccacgcgt

SEQ ID NO: 392 - RNA, 18 nt, synthetic construct
  source 1..18: mol_type = other RNA
aagggagccacgcgttac

SEQ ID NO: 393 - RNA, 18 nt, synthetic construct
  source 1..18: mol_type = other RNA
agcccaagggagccacgc

SEQ ID NO: 394 - RNA, 18 nt, synthetic construct
  source 1..18: mol_type = other RNA
cttccgggacagtgtgga

SEQ ID NO: 395 - RNA, 18 nt, synthetic construct
  source 1..18: mol_type = other RNA
ttcttggcagctggaaca

SEQ ID NO: 396 - RNA, 18 nt, synthetic construct
  source 1..18: mol_type = other RNA
gtagtcctggtggcatgc

SEQ ID NO: 397 - RNA, 18 nt, synthetic construct
  source 1..18: mol_type = other RNA
cccctgtagtcctggtgg

SEQ ID NO: 398 - RNA, 18 nt, synthetic construct
  source 1..18: mol_type = other RNA
ctgtagtcctggtggcat

SEQ ID NO: 399 - RNA, 18 nt, synthetic construct
  source 1..18: mol_type = other RNA
tggcatcacggacaatgc

SEQ ID NO: 400 - RNA, 18 nt, synthetic construct
  source 1..18: mol_type = other RNA
cgacaaagagattcagca

SEQ ID NO: 401 - RNA, 18 nt, synthetic construct
  source 1..18: mol_type = other RNA
gacggcgacaaagagatt

SEQ ID NO: 402 - RNA, 18 nt, synthetic construct
  source 1..18: mol_type = other RNA
atgatgacggcgacaaag

SEQ ID NO: 403 - RNA, 18 nt, synthetic construct
  source 1..18: mol_type = other RNA
tgtccatgatgacggcga

SEQ ID NO: 404 - RNA, 18 nt, synthetic construct
  source 1..18: mol_type = other RNA
aaagttgtccatgatgac

SEQ ID NO: 405 - RNA, 18 nt, synthetic construct
  source 1..18: mol_type = other RNA
gacttgtgggtgttctca

SEQ ID NO: 406 - RNA, 18 nt, synthetic construct
  source 1..18: mol_type = other RNA
gagtggacttgtgggtgt

SEQ ID NO: 407 - RNA, 18 nt, synthetic construct
  source 1..18: mol_type = other RNA
cgctggagtggacttgtg

SEQ ID NO: 408 - RNA, 18 nt, synthetic construct
  source 1..18: mol_type = other RNA
tagctcgctggagtggac

SEQ ID NO: 409 - RNA, 18 nt, synthetic construct
  source 1..18: mol_type = other RNA
gtcttctgccttgggctt

SEQ ID NO: 410 - RNA, 18 nt, synthetic construct
  source 1..18: mol_type = other RNA
gataggtcttctgccttg

SEQ ID NO: 411 - RNA, 18 nt, synthetic construct
  source 1..18: mol_type = other RNA
atctaaaatctgcttctt

SEQ ID NO: 412 - RNA, 18 nt, synthetic construct
  source 1..18: mol_type = other RNA
atgcagataggtcttctg

SEQ ID NO: 413 - RNA, 18 nt, synthetic construct
  source 1..18: mol_type = other RNA
gtcttctgccttgggctt

SEQ ID NO: 414 - RNA, 18 nt, synthetic construct
  source 1..18: mol_type = other RNA
ctcaaatgcagataggtc

SEQ ID NO: 415 - RNA, 18 nt, synthetic construct
  source 1..18: mol_type = other RNA
tcttttccatgtattcca

SEQ ID NO: 416 - RNA, 18 nt, synthetic construct
  source 1..18: mol_type = other RNA
accacgttcaccccgttc

SEQ ID NO: 417 - RNA, 18 nt, synthetic construct
  source 1..18: mol_type = other RNA
ggccggtcaggagggcgg

SEQ ID NO: 418 - RNA, 18 nt, synthetic construct
  source 1..18: mol_type = other RNA
gagccggccggtcaggag

SEQ ID NO: 419 - RNA, 18 nt, synthetic construct
  source 1..18: mol_type = other RNA
accgggagccggccggtc

SEQ ID NO: 420 - RNA, 18 nt, synthetic construct
  source 1..18: mol_type = other RNA
tccgaaccgggagccggc

SEQ ID NO: 421 - RNA, 18 nt, synthetic construct
  source 1..18: mol_type = other RNA
tccgaaccgggagccggc

SEQ ID NO: 422 - RNA, 21 nt, synthetic construct
  source 1..21: mol_type = other RNA
ccagaaaaaataaatactttc

SEQ ID NO: 423 - RNA, 18 nt, synthetic construct
  source 1..18: mol_type = other RNA
aatgttccagaaaaaata

SEQ ID NO: 424 - RNA, 18 nt, synthetic construct
  source 1..18: mol_type = other RNA
tctaaatgttccagaaaa

SEQ ID NO: 425 - RNA, 18 nt, synthetic construct
  source 1..18: mol_type = other RNA
ccactgttcatagggatc

SEQ ID NO: 426 - RNA, 26 nt, synthetic construct
  source 1..26: mol_type = other RNA
ccatacctgtataggagagattcata

SEQ ID NO: 427 - RNA, 26 nt, synthetic construct
  source 1..26: mol_type = other RNA
ccatacctgtataggagagaattaga

SEQ ID NO: 428 - RNA, 32 nt, synthetic construct
  source 1..32: mol_type = other RNA
ccatacctgtataggagagaattagattcata

SEQ ID NO: 429 - RNA, 25 nt, synthetic construct
  source 1..25: mol_type = other RNA
atcctctttggtaacctcacctcac

SEQ ID NO: 430 - RNA, 30 nt, synthetic construct
  source 1..30: mol_type = other RNA
tctccacggtcgcccttcagcccgcgttct

SEQ ID NO: 431 - RNA, 25 nt, synthetic construct
  source 1..25: mol_type = other RNA
tctccacggtcgcccttcagcccgc

SEQ ID NO: 432 - RNA, 25 nt, synthetic construct
  source 1..25: mol_type = other RNA
cctttctctccccgttctccctgaa

SEQ ID NO: 433 - RNA, 25 nt, synthetic construct
  source 1..25: mol_type = other RNA
cctttctctccccattctccctgaa

SEQ ID NO: 434 - RNA, 26 nt, synthetic construct
  source 1..26: mol_type = other RNA
ccacctgttctccacgttctcctttc

SEQ ID NO: 435 - RNA, 24 nt, synthetic construct
  source 1..24: mol_type = other RNA
ggcctcttggaccctgcagaccct

SEQ ID NO: 436 - RNA, 24 nt, synthetic construct
  source 1..24: mol_type = other RNA
ggcctcttggaccctacagaccct

SEQ ID NO: 437 - RNA, 22 nt, synthetic construct
  source 1..22: mol_type = other RNA
acctacccagttcggaatgatc

SEQ ID NO: 438 - RNA, 18 nt, synthetic construct
  source 1..18: mol_type = other RNA
ctacccagttcggaatga

SEQ ID NO: 439 - RNA, 15 nt, synthetic construct
  source 1..15: mol_type = other RNA
tacccagttcggaat

SEQ ID NO: 440 - RNA, 22 nt, synthetic construct
  source 1..22: mol_type = other RNA
atgtctcctctgttgtctaatg

SEQ ID NO: 441 - RNA, 22 nt, synthetic construct
  source 1..22: mol_type = other RNA
taacagcaaagacgatcattcc

SEQ ID NO: 442 - RNA, 22 nt, synthetic construct
  source 1..22: mol_type = other RNA
gaaacaactttttgtttgagaa

SEQ ID NO: 443 - RNA, 22 nt, synthetic construct
  source 1..22: mol_type = other RNA
gtttttccagagtgcacaacta

SEQ ID NO: 444 - RNA, 19 nt, synthetic construct
  source 1..19: mol_type = other RNA
agtccaagtgtggctcaaa

SEQ ID NO: 445 - RNA, 19 nt, synthetic construct
  source 1..19: mol_type = other RNA
tttgagccacacttggact

SEQ ID NO: 446 - RNA, 19 nt, synthetic construct
  source 1..19: mol_type = other RNA
ggctcaaaggataatatca

SEQ ID NO: 447 - RNA, 19 nt, synthetic construct
  source 1..19: mol_type = other RNA
tgatattatcctttgagcc

SEQ ID NO: 448 - RNA, 19 nt, synthetic construct
  source 1..19: mol_type = other RNA
gcaacgtccagtccaagtg

SEQ ID NO: 449 - RNA, 19 nt, synthetic construct
  source 1..19: mol_type = other RNA
cacttggactggacgttgc

SEQ ID NO: 450 - RNA, 25 nt, synthetic construct
  source 1..25: mol_type = other RNA
agacagacctgatcacctacctggt

SEQ ID NO: 451 - RNA, 25 nt, synthetic construct
  source 1..25: mol_type = other RNA
ggtatctgccagaaaatgcacagga

SEQ ID NO: 452 - RNA, 25 nt, synthetic construct
  source 1..25: mol_type = other RNA
ctaccagcctaccatgttaccttga

SEQ ID NO: 453 - RNA, 25 nt, synthetic construct
  source 1..25: mol_type = other RNA
ccacaggcgttgcactttgcaatgc

SEQ ID NO: 454 - RNA, 25 nt, synthetic construct
  source 1..25: mol_type = other RNA
cacaggcgttgcactttgcaatgct

SEQ ID NO: 455 - RNA, 25 nt, synthetic construct
  source 1..25: mol_type = other RNA
acaggcgttgcactttgcaatgctg

SEQ ID NO: 456 - RNA, 25 nt, synthetic construct
  source 1..25: mol_type = other RNA
caggcgttgcactttgcaatgctgc

SEQ ID NO: 457 - RNA, 25 nt, synthetic construct
  source 1..25: mol_type = other RNA
caggcgttgcactttgcaatgctgc

SEQ ID NO: 458 - RNA, 25 nt, synthetic construct
  source 1..25: mol_type = other RNA
aggcgttgcactttgcaatgctgct

SEQ ID NO: 459 - RNA, 25 nt, synthetic construct
  source 1..25: mol_type = other RNA
ggcgttgcactttgcaatgctgctg

SEQ ID NO: 460 - RNA, 25 nt, synthetic construct
  source 1..25: mol_type = other RNA
gcgttgcactttgcaatgctgctgt

SEQ ID NO: 461 - RNA, 25 nt, synthetic construct
  source 1..25: mol_type = other RNA
cgttgcactttgcaatgctgctgtc

SEQ ID NO: 462 - RNA, 23 nt, synthetic construct
  source 1..23: mol_type = other RNA
cgttgcactttgcaatgctgctg

SEQ ID NO: 463 - RNA, 25 nt, synthetic construct
  source 1..25: mol_type = other RNA
gttgcactttgcaatgctgctgtct

SEQ ID NO: 464 - RNA, 25 nt, synthetic construct
  source 1..25: mol_type = other RNA
ttgcactttgcaatgctgctgtctt

SEQ ID NO: 465 - RNA, 25 nt, synthetic construct
  source 1..25: mol_type = other RNA
tgcactttgcaatgctgctgtcttc

SEQ ID NO: 466 - RNA, 25 nt, synthetic construct
  source 1..25: mol_type = other RNA
gcactttgcaatgctgctgtcttct

SEQ ID NO: 467 - RNA, 25 nt, synthetic construct
  source 1..25: mol_type = other RNA
cactttgcaatgctgctgtcttctt

SEQ ID NO: 468 - RNA, 25 nt, synthetic construct
  source 1..25: mol_type = other RNA
actttgcaatgctgctgtcttcttg

SEQ ID NO: 469 - RNA, 25 nt, synthetic construct
  source 1..25: mol_type = other RNA
ctttgcaatgctgctgtcttcttgc

SEQ ID NO: 470 - RNA, 25 nt, synthetic construct
  source 1..25: mol_type = other RNA
tttgcaatgctgctgtcttcttgct

SEQ ID NO: 471 - RNA, 25 nt, synthetic construct
  source 1..25: mol_type = other RNA
ttgcaatgctgctgtcttcttgcta

SEQ ID NO: 472 - RNA, 25 nt, synthetic construct
  source 1..25: mol_type = other RNA
tgcaatgctgctgtcttcttgctat

SEQ ID NO: 473 - RNA, 25 nt, synthetic construct
  source 1..25: mol_type = other RNA
gcaatgctgctgtcttcttgctatg

SEQ ID NO: 474 - RNA, 25 nt, synthetic construct
  source 1..25: mol_type = other RNA
caatgctgctgtcttcttgctatga

SEQ ID NO: 475 - RNA, 25 nt, synthetic construct
  source 1..25: mol_type = other RNA
aatgctgctgtcttcttgctatgaa

SEQ ID NO: 476 - RNA, 25 nt, synthetic construct
  source 1..25: mol_type = other RNA
atgctgctgtcttcttgctatgaat

SEQ ID NO: 477 - RNA, 25 nt, synthetic construct
  source 1..25: mol_type = other RNA
tgctgctgtcttcttgctatgaata

SEQ ID NO: 478 - RNA, 25 nt, synthetic construct
  source 1..25: mol_type = other RNA
gctgctgtcttcttgctatgaataa

SEQ ID NO: 479 - RNA, 25 nt, synthetic construct
  source 1..25: mol_type = other RNA
ctgctgtcttcttgctatgaataat

SEQ ID NO: 480 - RNA, 25 nt, synthetic construct
  source 1..25: mol_type = other RNA
tgctgtcttcttgctatgaataatg

SEQ ID NO: 481 - RNA, 25 nt, synthetic construct
  source 1..25: mol_type = other RNA
gctgtcttcttgctatgaataatgt

SEQ ID NO: 482 - RNA, 25 nt, synthetic construct
  source 1..25: mol_type = other RNA
ctgtcttcttgctatgaataatgtc

SEQ ID NO: 483 - RNA, 25 nt, synthetic construct
  source 1..25: mol_type = other RNA
tgtcttcttgctatgaataatgtca

SEQ ID NO: 484 - RNA, 25 nt, synthetic construct
  source 1..25: mol_type = other RNA
gtcttcttgctatgaataatgtcaa

SEQ ID NO: 485 - RNA, 25 nt, synthetic construct
  source 1..25: mol_type = other RNA
tcttcttgctatgaataatgtcaat

SEQ ID NO: 486 - RNA, 25 nt, synthetic construct
  source 1..25: mol_type = other RNA
cttcttgctatgaataatgtcaatc

SEQ ID NO: 487 - RNA, 25 nt, synthetic construct
  source 1..25: mol_type = other RNA
ttcttgctatgaataatgtcaatcc

SEQ ID NO: 488 - RNA, 25 nt, synthetic construct
  source 1..25: mol_type = other RNA
tcttgctatgaataatgtcaatccg

SEQ ID NO: 489 - RNA, 25 nt, synthetic construct
  source 1..25: mol_type = other RNA
cttgctatgaataatgtcaatccga

SEQ ID NO: 490 - RNA, 25 nt, synthetic construct
  source 1..25: mol_type = other RNA
ttgctatgaataatgtcaatccgac

SEQ ID NO: 491 - RNA, 25 nt, synthetic construct
  source 1..25: mol_type = other RNA
tgctatgaataatgtcaatccgacc

SEQ ID NO: 492 - RNA, 25 nt, synthetic construct
  source 1..25: mol_type = other RNA
gctatgaataatgtcaatccgacct

SEQ ID NO: 493 - RNA, 25 nt, synthetic construct
  source 1..25: mol_type = other RNA
ctatgaataatgtcaatccgacctg

SEQ ID NO: 494 - RNA, 25 nt, synthetic construct
  source 1..25: mol_type = other RNA
tatgaataatgtcaatccgacctga

SEQ ID NO: 495 - RNA, 25 nt, synthetic construct
  source 1..25: mol_type = other RNA
atgaataatgtcaatccgacctgag

SEQ ID NO: 496 - RNA, 25 nt, synthetic construct
  source 1..25: mol_type = other RNA
tgaataatgtcaatccgacctgagc

SEQ ID NO: 497 - RNA, 25 nt, synthetic construct
  source 1..25: mol_type = other RNA
gaataatgtcaatccgacctgagct

SEQ ID NO: 498 - RNA, 25 nt, synthetic construct
  source 1..25: mol_type = other RNA
aataatgtcaatccgacctgagctt

SEQ ID NO: 499 - RNA, 25 nt, synthetic construct
  source 1..25: mol_type = other RNA
ataatgtcaatccgacctgagcttt

SEQ ID NO: 500 - RNA, 25 nt, synthetic construct
  source 1..25: mol_type = other RNA
taatgtcaatccgacctgagctttg

SEQ ID NO: 501 - RNA, 25 nt, synthetic construct
  source 1..25: mol_type = other RNA
aatgtcaatccgacctgagctttgt

SEQ ID NO: 502 - RNA, 25 nt, synthetic construct
  source 1..25: mol_type = other RNA
atgtcaatccgacctgagctttgtt

SEQ ID NO: 503 - RNA, 25 nt, synthetic construct
  source 1..25: mol_type = other RNA
tgtcaatccgacctgagctttgttg

SEQ ID NO: 504 - RNA, 25 nt, synthetic construct
  source 1..25: mol_type = other RNA
gtcaatccgacctgagctttgttgt

SEQ ID NO: 505 - RNA, 25 nt, synthetic construct
  source 1..25: mol_type = other RNA
tcaatccgacctgagctttgttgta

SEQ ID NO: 506 - RNA, 25 nt, synthetic construct
  source 1..25: mol_type = other RNA
caatccgacctgagctttgttgtag

SEQ ID NO: 507 - RNA, 25 nt, synthetic construct
  source 1..25: mol_type = other RNA
aatccgacctgagctttgttgtaga

SEQ ID NO: 508 - RNA, 25 nt, synthetic construct
  source 1..25: mol_type = other RNA
atccgacctgagctttgttgtagac

SEQ ID NO: 509 - RNA, 25 nt, synthetic construct
  source 1..25: mol_type = other RNA
tccgacctgagctttgttgtagact

SEQ ID NO: 510 - RNA, 25 nt, synthetic construct
  source 1..25: mol_type = other RNA
ccgacctgagctttgttgtagacta

SEQ ID NO: 511 - RNA, 20 nt, synthetic construct
  source 1..20: mol_type = other RNA
cgacctgagctttgttgtag

SEQ ID NO: 512 - RNA, 25 nt, synthetic construct
  source 1..25: mol_type = other RNA
cgacctgagctttgttgtagactat

SEQ ID NO: 513 - RNA, 25 nt, synthetic construct
  source 1..25: mol_type = other RNA
gacctgagctttgttgtagactatc

SEQ ID NO: 514 - RNA, 25 nt, synthetic construct
  source 1..25: mol_type = other RNA
acctgagctttgttgtagactatca

SEQ ID NO: 515 - RNA, 23 nt, synthetic construct
  source 1..23: mol_type = other RNA
cctgagctttgttgtagactatc

SEQ ID NO: 516 - RNA, 22 nt, synthetic construct
  source 1..22: mol_type = other RNA
gcaatgctgctgtcttcttgct

SEQ ID NO: 517 - RNA, 22 nt, synthetic construct
  source 1..22: mol_type = other RNA
aacaaaatgtacaaggaccgac

SEQ ID NO: 518 - RNA, 22 nt, synthetic construct
  source 1..22: mol_type = other RNA
tgcaaagtgcaacgcctgtgga

SEQ ID NO: 519 - RNA, 22 nt, synthetic construct
  source 1..22: mol_type = other RNA
atagtctacaacaaagctcagg

SEQ ID NO: 520 - RNA, 22 nt, synthetic construct
  source 1..22: mol_type = other RNA
ctgttttaaaatttttatatta

SEQ ID NO: 521 - RNA, 22 nt, synthetic construct
  source 1..22: mol_type = other RNA
atatatgtgttacctacccttg

SEQ ID NO: 522 - RNA, 22 nt, synthetic construct
  source 1..22: mol_type = other RNA
acattttgttaactttttccca

SEQ ID NO: 523 - RNA, 22 nt, synthetic construct
  source 1..22: mol_type = other RNA
ctttttcccattggaaatcaag

SEQ ID NO: 524 - RNA, 22 nt, synthetic construct
  source 1..22: mol_type = other RNA
tttttcccattggaaatcaagc

SEQ ID NO: 525 - RNA, 22 nt, synthetic construct
  source 1..22: mol_type = other RNA
tcctgtagcttcaccctttcca

SEQ ID NO: 526 - RNA, 22 nt, synthetic construct
  source 1..22: mol_type = other RNA
aatgtacaaggaccgacaaggg

SEQ ID NO: 527 - RNA, 22 nt, synthetic construct
  source 1..22: mol_type = other RNA
acaaaatgtacaaggaccgaca

SEQ ID NO: 528 - RNA, 22 nt, synthetic construct
  source 1..22: mol_type = other RNA
aacaaaatgtacaaggaccgac

SEQ ID NO: 529 - RNA, 22 nt, synthetic construct
  source 1..22: mol_type = other RNA
ggaaaaagttaacaaaatgtac

SEQ ID NO: 530 - RNA, 22 nt, synthetic construct
  source 1..22: mol_type = other RNA
tctctcccagcttgatttccaa

SEQ ID NO: 531 - RNA, 22 nt, synthetic construct
  source 1..22: mol_type = other RNA
ctctctcccagcttgatttcca

SEQ ID NO: 532 - RNA, 22 nt, synthetic construct
  source 1..22: mol_type = other RNA
gcctgtggaaagggtgaagcta

SEQ ID NO: 533 - RNA, 22 nt, synthetic construct
  source 1..22: mol_type = other RNA
gcaaagtgcaacgcctgtggaa

SEQ ID NO: 534 - RNA, 22 nt, synthetic construct
  source 1..22: mol_type = other RNA
tgcaaagtgcaacgcctgtgga

SEQ ID NO: 535 - RNA, 22 nt, synthetic construct
  source 1..22: mol_type = other RNA
agcattgcaaagtgcaacgcct

SEQ ID NO: 536 - RNA, 22 nt, synthetic construct
  source 1..22: mol_type = other RNA
atagtctacaacaaagctcagg

SEQ ID NO: 537 - RNA, 22 nt, synthetic construct
  source 1..22: mol_type = other RNA
aaagatagtctacaacaaagct

SEQ ID NO: 538 - RNA, 16 nt, synthetic construct
  source 1..16: mol_type = other RNA
gtggctaacagaagct

SEQ ID NO: 539 - RNA, 17 nt, synthetic construct
  source 1..17: mol_type = other RNA
gggaacatgctaaatac

SEQ ID NO: 540 - RNA, 18 nt, synthetic construct
  source 1..18: mol_type = other RNA
agacacaaattcctgaga

SEQ ID NO: 541 - RNA, 11 nt, synthetic construct
  source 1..11: mol_type = other RNA
ctgttgagaaa

SEQ ID NO: 542 - RNA, 22 nt, synthetic construct
  source 1..22: mol_type = other RNA
atttagcatgttcccaattctc

SEQ ID NO: 543 - RNA, 22 nt, synthetic construct
  source 1..22: mol_type = other RNA
aatcgcctgcaggtaaaagcat

SEQ ID NO: 544 - RNA, 22 nt, synthetic construct
  source 1..22: mol_type = other RNA
tctcagaaagacacaaattcct

SEQ ID NO: 545 - RNA, 22 nt, synthetic construct
  source 1..22: mol_type = other RNA
tatttagcatgttcccaattct

SEQ ID NO: 546 - RNA, 22 nt, synthetic construct
  source 1..22: mol_type = other RNA
aacagatctgtcaaatcgcctg

SEQ ID NO: 547 - RNA, 22 nt, synthetic construct
  source 1..22: mol_type = other RNA
aatcgcctgcaggtaaaagcat

SEQ ID NO: 548 - RNA, 22 nt, synthetic construct
  source 1..22: mol_type = other RNA
tgctaaatacaaatggtatctt

SEQ ID NO: 549 - RNA, 22 nt, synthetic construct
  source 1..22: mol_type = other RNA
attgggaacatgctaaatacaa

SEQ ID NO: 550 - RNA, 22 nt, synthetic construct
  source 1..22: mol_type = other RNA
aaagacacaaattcctgagaat

SEQ ID NO: 551 - RNA, 21 nt, synthetic construct
  source 1..21: mol_type = other RNA
gaaagacacaaattcctgaga

SEQ ID NO: 552 - RNA, 22 nt, synthetic construct
  source 1..22: mol_type = other RNA
atgatataaagatatttaatca

SEQ ID NO: 553 - RNA, 22 nt, synthetic construct
  source 1..22: mol_type = other RNA
ttgacagatctgttgagaaatg

SEQ ID NO: 554 - RNA, 22 nt, synthetic construct
  source 1..22: mol_type = other RNA
gatttgacagatctgttgagaa

SEQ ID NO: 555 - RNA, 22 nt, synthetic construct
  source 1..22: mol_type = other RNA
ttgatccatatgcttttacctg

SEQ ID NO: 556 - RNA, 25 nt, synthetic construct
  source 1..25: mol_type = other RNA
tttgccgctgcccaatgccatcctg

SEQ ID NO: 557 - RNA, 25 nt, synthetic construct
  source 1..25: mol_type = other RNA
tttgccgctgcccaatgccatcctg

SEQ ID NO: 558 - RNA, 25 nt, synthetic construct
  source 1..25: mol_type = other RNA
  modified_base 7: mod_base = i
tttgccnctgcccaatgccatcctg

SEQ ID NO: 559 - RNA, 22 nt, synthetic construct
  source 1..22: mol_type = other RNA
tagaatactggcatctgttttt

SEQ ID NO: 560 - RNA, 22 nt, synthetic construct
  source 1..22: mol_type = other RNA
tggcatctgtttttgaggattg

SEQ ID NO: 561 - RNA, 22 nt, synthetic construct
  source 1..22: mol_type = other RNA
ttgccgctgcccaatgccatcc

SEQ ID NO: 562 - RNA, 22 nt, synthetic construct
  source 1..22: mol_type = other RNA
gaggtagggcgacagatctaat

SEQ ID NO: 563 - RNA, 22 nt, synthetic construct
  source 1..22: mol_type = other RNA
tctacaggaaaaattgggaagc

SEQ ID NO: 564 - RNA, 22 nt, synthetic construct
  source 1..22: mol_type = other RNA
gcggcaaactgttgtcagaaca

SEQ ID NO: 565 - RNA, 22 nt, synthetic construct
  source 1..22: mol_type = other RNA
ttttggtatcttacaggaactc

SEQ ID NO: 566 - RNA, 22 nt, synthetic construct
  source 1..22: mol_type = other RNA
agacctcctgccaccgcagatt

SEQ ID NO: 567 - RNA, 22 nt, synthetic construct
  source 1..22: mol_type = other RNA
tcccaatttttcctgtagaata

SEQ ID NO: 568 - RNA, 22 nt, synthetic construct
  source 1..22: mol_type = other RNA
tagaatactggcatctgttttt

SEQ ID NO: 569 - RNA, 22 nt, synthetic construct
  source 1..22: mol_type = other RNA
tttgccgctgcccaatgccatc

SEQ ID NO: 570 - RNA, 22 nt, synthetic construct
  source 1..22: mol_type = other RNA
ggagttcctgtaagataccaaa

SEQ ID NO: 571 - RNA, 22 nt, synthetic construct
  source 1..22: mol_type = other RNA
agaggtagggcgacagatctaa

SEQ ID NO: 572 - RNA, 22 nt, synthetic construct
  source 1..22: mol_type = other RNA
ctgtcagacagaaaaaagaggt

SEQ ID NO: 573 - RNA, 22 nt, synthetic construct
  source 1..22: mol_type = other RNA
gctgtcagacagaaaaaagagg

SEQ ID NO: 574 - RNA, 22 nt, synthetic construct
  source 1..22: mol_type = other RNA
aacagctgtcagacagaaaaaa

SEQ ID NO: 575 - RNA, 22 nt, synthetic construct
  source 1..22: mol_type = other RNA
aagcctgaatctgcggtggcag

SEQ ID NO: 576 - RNA, 22 nt, synthetic construct
  source 1..22: mol_type = other RNA
gggaagcctgaatctgcggtgg

SEQ ID NO: 577 - RNA, 22 nt, synthetic construct
  source 1..22: mol_type = other RNA
aattgggaagcctgaatctgcg

SEQ ID NO: 578 - RNA, 22 nt, synthetic construct
  source 1..22: mol_type = other RNA
aaaaattgggaagcctgaatct

SEQ ID NO: 579 - RNA, 22 nt, synthetic construct
  source 1..22: mol_type = other RNA
gccagtattctacaggaaaaat

SEQ ID NO: 580 - RNA, 22 nt, synthetic construct
  source 1..22: mol_type = other RNA
tgccagtattctacaggaaaaa

SEQ ID NO: 581 - RNA, 22 nt, synthetic construct
  source 1..22: mol_type = other RNA
aaaaacagatgccagtattcta

SEQ ID NO: 582 - RNA, 22 nt, synthetic construct
  source 1..22: mol_type = other RNA
tgtcagaacattgaatgcaact

SEQ ID NO: 583 - RNA, 22 nt, synthetic construct
  source 1..22: mol_type = other RNA
ttgtcagaacattgaatgcaac

SEQ ID NO: 584 - RNA, 22 nt, synthetic construct
  source 1..22: mol_type = other RNA
gttgtcagaacattgaatgcaa

SEQ ID NO: 585 - RNA, 22 nt, synthetic construct
  source 1..22: mol_type = other RNA
aactccaggatggcattgggca

SEQ ID NO: 586 - RNA, 22 nt, synthetic construct
  source 1..22: mol_type = other RNA
tacaggaactccaggatggcat

SEQ ID NO: 587 - RNA, 22 nt, synthetic construct
  source 1..22: mol_type = other RNA
ttacaggaactccaggatggca

SEQ ID NO: 588 - RNA, 22 nt, synthetic construct
  source 1..22: mol_type = other RNA
ggtatcttacaggaactccagg

SEQ ID NO: 589 - RNA, 22 nt, synthetic construct
  source 1..22: mol_type = other RNA
ttttggtatcttacaggaactc

SEQ ID NO: 590 - RNA, 22 nt, synthetic construct
  source 1..22: mol_type = other RNA
gttttgcctttttggtatctta

SEQ ID NO: 591 - RNA, 22 nt, synthetic construct
  source 1..22: mol_type = other RNA
tcttttctcaaataaaaagaca

SEQ ID NO: 592 - RNA, 25 nt, synthetic construct
  source 1..25: mol_type = other RNA
gcttttcttttagttgctgctcttt

SEQ ID NO: 593 - RNA, 25 nt, synthetic construct
  source 1..25: mol_type = other RNA
cttttcttttagttgctgctctttt

SEQ ID NO: 594 - RNA, 25 nt, synthetic construct
  source 1..25: mol_type = other RNA
ttttcttttagttgctgctcttttc

SEQ ID NO: 595 - RNA, 25 nt, synthetic construct
  source 1..25: mol_type = other RNA
tttcttttagttgctgctcttttcc

SEQ ID NO: 596 - RNA, 25 nt, synthetic construct
  source 1..25: mol_type = other RNA
ttcttttagttgctgctcttttcca

SEQ ID NO: 597 - RNA, 25 nt, synthetic construct
  source 1..25: mol_type = other RNA
tcttttagttgctgctcttttccag

SEQ ID NO: 598 - RNA, 25 nt, synthetic construct
  source 1..25: mol_type = other RNA
cttttagttgctgctcttttccagg

SEQ ID NO: 599 - RNA, 25 nt, synthetic construct
  source 1..25: mol_type = other RNA
ttttagttgctgctcttttccaggt

SEQ ID NO: 600 - RNA, 25 nt, synthetic construct
  source 1..25: mol_type = other RNA
tttagttgctgctcttttccaggtt

SEQ ID NO: 601 - RNA, 25 nt, synthetic construct
  source 1..25: mol_type = other RNA
ttagttgctgctcttttccaggttc

SEQ ID NO: 602 - RNA, 25 nt, synthetic construct
  source 1..25: mol_type = other RNA
tagttgctgctcttttccaggttca

SEQ ID NO: 603 - RNA, 25 nt, synthetic construct
  source 1..25: mol_type = other RNA
agttgctgctcttttccaggttcaa

SEQ ID NO: 604 - RNA, 25 nt, synthetic construct
  source 1..25: mol_type = other RNA
gttgctgctcttttccaggttcaag

SEQ ID NO: 605 - RNA, 25 nt, synthetic construct
  source 1..25: mol_type = other RNA
ttgctgctcttttccaggttcaagt

SEQ ID NO: 606 - RNA, 25 nt, synthetic construct
  source 1..25: mol_type = other RNA
tgctgctcttttccaggttcaagtg

SEQ ID NO: 607 - RNA, 25 nt, synthetic construct
  source 1..25: mol_type = other RNA
gctgctcttttccaggttcaagtgg

SEQ ID NO: 608 - RNA, 25 nt, synthetic construct
  source 1..25: mol_type = other RNA
ctgctcttttccaggttcaagtggg

SEQ ID NO: 609 - RNA, 25 nt, synthetic construct
  source 1..25: mol_type = other RNA
tgctcttttccaggttcaagtggga

SEQ ID NO: 610 - RNA, 25 nt, synthetic construct
  source 1..25: mol_type = other RNA
gctcttttccaggttcaagtgggac

SEQ ID NO: 611 - RNA, 25 nt, synthetic construct
  source 1..25: mol_type = other RNA
ctcttttccaggttcaagtgggata

SEQ ID NO: 612 - RNA, 25 nt, synthetic construct
  source 1..25: mol_type = other RNA
tcttttccaggttcaagtgggatac

SEQ ID NO: 613 - RNA, 20 nt, synthetic construct
  source 1..20: mol_type = other RNA
tcttttccaggttcaagtgg

SEQ ID NO: 614 - RNA, 25 nt, synthetic construct
  source 1..25: mol_type = other RNA
cttttccaggttcaagtgggatact

SEQ ID NO: 615 - RNA, 25 nt, synthetic construct
  source 1..25: mol_type = other RNA
ttttccaggttcaagtgggatacta

SEQ ID NO: 616 - RNA, 25 nt, synthetic construct
  source 1..25: mol_type = other RNA
tttccaggttcaagtgggatactag

SEQ ID NO: 617 - RNA, 25 nt, synthetic construct
  source 1..25: mol_type = other RNA
ttccaggttcaagtgggatactagc

SEQ ID NO: 618 - RNA, 25 nt, synthetic construct
  source 1..25: mol_type = other RNA
tccaggttcaagtgggatactagca

SEQ ID NO: 619 - RNA, 25 nt, synthetic construct
  source 1..25: mol_type = other RNA
ccaggttcaagtgggatactagcaa

SEQ ID NO: 620 - RNA, 25 nt, synthetic construct
  source 1..25: mol_type = other RNA
caggttcaagtgggatactagcaat

SEQ ID NO: 621 - RNA, 25 nt, synthetic construct
  source 1..25: mol_type = other RNA
aggttcaagtgggatactagcaatg

SEQ ID NO: 622 - RNA, 25 nt, synthetic construct
  source 1..25: mol_type = other RNA
ggttcaagtgggatactagcaatgt

SEQ ID NO: 623 - RNA, 25 nt, synthetic construct
  source 1..25: mol_type = other RNA
gttcaagtgggatactagcaatgtt

SEQ ID NO: 624 - RNA, 25 nt, synthetic construct
  source 1..25: mol_type = other RNA
ttcaagtgggatactagcaatgtta

SEQ ID NO: 625 - RNA, 25 nt, synthetic construct
  source 1..25: mol_type = other RNA
tcaagtgggatactagcaatgttat

SEQ ID NO: 626 - RNA, 25 nt, synthetic construct
  source 1..25: mol_type = other RNA
caagtgggatactagcaatgttatc

SEQ ID NO: 627 - RNA, 25 nt, synthetic construct
  source 1..25: mol_type = other RNA
aagtgggatactagcaatgttatct

SEQ ID NO: 628 - RNA, 25 nt, synthetic construct
  source 1..25: mol_type = other RNA
agtgggatactagcaatgttatctg

SEQ ID NO: 629 - RNA, 25 nt, synthetic construct
  source 1..25: mol_type = other RNA
gtgggatactagcaatgttatctgc

SEQ ID NO: 630 - RNA, 25 nt, synthetic construct
  source 1..25: mol_type = other RNA
tgggatactagcaatgttatctgct

SEQ ID NO: 631 - RNA, 25 nt, synthetic construct
  source 1..25: mol_type = other RNA
gggatactagcaatgttatctgctt

SEQ ID NO: 632 - RNA, 25 nt, synthetic construct
  source 1..25: mol_type = other RNA
ggatactagcaatgttatctgcttc

SEQ ID NO: 633 - RNA, 25 nt, synthetic construct
  source 1..25: mol_type = other RNA
gatactagcaatgttatctgcttcc

SEQ ID NO: 634 - RNA, 25 nt, synthetic construct
  source 1..25: mol_type = other RNA
atactagcaatgttatctgcttcct

SEQ ID NO: 635 - RNA, 25 nt, synthetic construct
  source 1..25: mol_type = other RNA
tactagcaatgttatctgcttcctc

SEQ ID NO: 636 - RNA, 25 nt, synthetic construct
  source 1..25: mol_type = other RNA
actagcaatgttatctgcttcctcc

SEQ ID NO: 637 - RNA, 25 nt, synthetic construct
  source 1..25: mol_type = other RNA
ctagcaatgttatctgcttcctcca

SEQ ID NO: 638 - RNA, 25 nt, synthetic construct
  source 1..25: mol_type = other RNA
tagcaatgttatctgcttcctccaa

SEQ ID NO: 639 - RNA, 25 nt, synthetic construct
  source 1..25: mol_type = other RNA
agcaatgttatctgcttcctccaac

SEQ ID NO: 640 - RNA, 25 nt, synthetic construct
  source 1..25: mol_type = other RNA
gcaatgttatctgcttcctccaacc

SEQ ID NO: 641 - RNA, 25 nt, synthetic construct
  source 1..25: mol_type = other RNA
caatgttatctgcttcctccaacca

SEQ ID NO: 642 - RNA, 25 nt, synthetic construct
  source 1..25: mol_type = other RNA
aatgttatctgcttcctccaaccat

SEQ ID NO: 643 - RNA, 25 nt, synthetic construct
  source 1..25: mol_type = other RNA
atgttatctgcttcctccaaccata

SEQ ID NO: 644 - RNA, 25 nt, synthetic construct
  source 1..25: mol_type = other RNA
tgttatctgcttcctccaaccataa

SEQ ID NO: 645 - RNA, 25 nt, synthetic construct
  source 1..25: mol_type = other RNA
ccaatagtggtcagtccaggagcta

SEQ ID NO: 646 - RNA, 25 nt, synthetic construct
  source 1..25: mol_type = other RNA
caatagtggtcagtccaggagctag

SEQ ID NO: 647 - RNA, 25 nt, synthetic construct
  source 1..25: mol_type = other RNA
aatagtggtcagtccaggagctagg

SEQ ID NO: 648 - RNA, 25 nt, synthetic construct
  source 1..25: mol_type = other RNA
atagtggtcagtccaggagctaggt

SEQ ID NO: 649 - RNA, 21 nt, synthetic construct
  source 1..21: mol_type = other RNA
atagtggtcagtccaggagct

SEQ ID NO: 650 - RNA, 25 nt, synthetic construct
  source 1..25: mol_type = other RNA
tagtggtcagtccaggagctaggtc

SEQ ID NO: 651 - RNA, 25 nt, synthetic construct
  source 1..25: mol_type = other RNA
agtggtcagtccaggagctaggtca

SEQ ID NO: 652 - RNA, 25 nt, synthetic construct
  source 1..25: mol_type = other RNA
gtggtcagtccaggagctaggtcag

SEQ ID NO: 653 - RNA, 25 nt, synthetic construct
  source 1..25: mol_type = other RNA
tggtcagtccaggagctaggtcagg

SEQ ID NO: 654 - RNA, 25 nt, synthetic construct
  source 1..25: mol_type = other RNA
ggtcagtccaggagctaggtcaggc

SEQ ID NO: 655 - RNA, 25 nt, synthetic construct
  source 1..25: mol_type = other RNA
gtcagtccaggagctaggtcaggct

SEQ ID NO: 656 - RNA, 25 nt, synthetic construct
  source 1..25: mol_type = other RNA
tcagtccaggagctaggtcaggctg

SEQ ID NO: 657 - RNA, 25 nt, synthetic construct
  source 1..25: mol_type = other RNA
cagtccaggagctaggtcaggctgc

SEQ ID NO: 658 - RNA, 25 nt, synthetic construct
  source 1..25: mol_type = other RNA
agtccaggagctaggtcaggctgct

SEQ ID NO: 659 - RNA, 25 nt, synthetic construct
  source 1..25: mol_type = other RNA
gtccaggagctaggtcaggctgctt

SEQ ID NO: 660 - RNA, 25 nt, synthetic construct
  source 1..25: mol_type = other RNA
tccaggagctaggtcaggctgcttt

SEQ ID NO: 661 - RNA, 25 nt, synthetic construct
  source 1..25: mol_type = other RNA
ccaggagctaggtcaggctgctttg

SEQ ID NO: 662 - RNA, 25 nt, synthetic construct
  source 1..25: mol_type = other RNA
caggagctaggtcaggctgctttgc

SEQ ID NO: 663 - RNA, 25 nt, synthetic construct
  source 1..25: mol_type = other RNA
aggagctaggtcaggctgctttgcc

SEQ ID NO: 664 - RNA, 25 nt, synthetic construct
  source 1..25: mol_type = other RNA
ggagctaggtcaggctgctttgccc

SEQ ID NO: 665 - RNA, 25 nt, synthetic construct
  source 1..25: mol_type = other RNA
gagctaggtcaggctgctttgccct

SEQ ID NO: 666 - RNA, 25 nt, synthetic construct
  source 1..25: mol_type = other RNA
agctaggtcaggctgctttgccctc

SEQ ID NO: 667 - RNA, 25 nt, synthetic construct
  source 1..25: mol_type = other RNA
gctaggtcaggctgctttgccctca

SEQ ID NO: 668 - RNA, 25 nt, synthetic construct
  source 1..25: mol_type = other RNA
ctaggtcaggctgctttgccctcag

SEQ ID NO: 669 - RNA, 25 nt, synthetic construct
  source 1..25: mol_type = other RNA
taggtcaggctgctttgccctcagc

SEQ ID NO: 670 - RNA, 25 nt, synthetic construct
  source 1..25: mol_type = other RNA
aggtcaggctgctttgccctcagct

SEQ ID NO: 671 - RNA, 25 nt, synthetic construct
  source 1..25: mol_type = other RNA
ggtcaggctgctttgccctcagctc

SEQ ID NO: 672 - RNA, 25 nt, synthetic construct
  source 1..25: mol_type = other RNA
gtcaggctgctttgccctcagctct

SEQ ID NO: 673 - RNA, 25 nt, synthetic construct
  source 1..25: mol_type = other RNA
tcaggctgctttgccctcagctctt

SEQ ID NO: 674 - RNA, 25 nt, synthetic construct
  source 1..25: mol_type = other RNA
caggctgctttgccctcagctcttg

SEQ ID NO: 675 - RNA, 25 nt, synthetic construct
  source 1..25: mol_type = other RNA
aggctgctttgccctcagctcttga

SEQ ID NO: 676 - RNA, 25 nt, synthetic construct
  source 1..25: mol_type = other RNA
ggctgctttgccctcagctcttgaa

SEQ ID NO: 677 - RNA, 25 nt, synthetic construct
  source 1..25: mol_type = other RNA
gctgctttgccctcagctcttgaag

SEQ ID NO: 678 - RNA, 25 nt, synthetic construct
  source 1..25: mol_type = other RNA
ctgctttgccctcagctcttgaagt

SEQ ID NO: 679 - RNA, 25 nt, synthetic construct
  source 1..25: mol_type = other RNA
tgctttgccctcagctcttgaagta

SEQ ID NO: 680 - RNA, 25 nt, synthetic construct
  source 1..25: mol_type = other RNA
gctttgccctcagctcttgaagtaa

SEQ ID NO: 681 - RNA, 25 nt, synthetic construct
  source 1..25: mol_type = other RNA
ctttgccctcagctcttgaagtaaa

SEQ ID NO: 682 - RNA, 25 nt, synthetic construct
  source 1..25: mol_type = other RNA
tttgccctcagctcttgaagtaaac

SEQ ID NO: 683 - RNA, 25 nt, synthetic construct
  source 1..25: mol_type = other RNA
ttgccctcagctcttgaagtaaacg

SEQ ID NO: 684 - RNA, 25 nt, synthetic construct
  source 1..25: mol_type = other RNA
tgccctcagctcttgaagtaaacgg

SEQ ID NO: 685 - RNA, 25 nt, synthetic construct
  source 1..25: mol_type = other RNA
gccctcagctcttgaagtaaacggt

SEQ ID NO: 686 - RNA, 25 nt, synthetic construct
  source 1..25: mol_type = other RNA
ccctcagctcttgaagtaaacggtt

SEQ ID NO: 687 - RNA, 20 nt, synthetic construct
  source 1..20: mol_type = other RNA
cctcagctcttgaagtaaac

SEQ ID NO: 688 - RNA, 21 nt, synthetic construct
  source 1..21: mol_type = other RNA
cctcagctcttgaagtaaacg

SEQ ID NO: 689 - RNA, 20 nt, synthetic construct
  source 1..20: mol_type = other RNA
ctcagctcttgaagtaaacg

SEQ ID NO: 690 - RNA, 25 nt, synthetic construct
  source 1..25: mol_type = other RNA
cctcagctcttgaagtaaacggttt

SEQ ID NO: 691 - RNA, 25 nt, synthetic construct
  source 1..25: mol_type = other RNA
ctcagctcttgaagtaaacggttta

SEQ ID NO: 692 - RNA, 25 nt, synthetic construct
  source 1..25: mol_type = other RNA
tcagctcttgaagtaaacggtttac

SEQ ID NO: 693 - RNA, 25 nt, synthetic construct
  source 1..25: mol_type = other RNA
cagctcttgaagtaaacggtttacc

SEQ ID NO: 694 - RNA, 25 nt, synthetic construct
  source 1..25: mol_type = other RNA
agctcttgaagtaaacggtttaccg

SEQ ID NO: 695 - RNA, 25 nt, synthetic construct
  source 1..25: mol_type = other RNA
gctcttgaagtaaacggtttaccgc

SEQ ID NO: 696 - RNA, 25 nt, synthetic construct
  source 1..25: mol_type = other RNA
ctcttgaagtaaacggtttaccgcc

SEQ ID NO: 697 - RNA, 22 nt, synthetic construct
  source 1..22: mol_type = other RNA
actattggagcctgtaagtata

SEQ ID NO: 698 - RNA, 22 nt, synthetic construct
  source 1..22: mol_type = other RNA
aggaagttagaagatctgagct

SEQ ID NO: 699 - RNA, 22 nt, synthetic construct
  source 1..22: mol_type = other RNA
agaatgggatccagtatactta

SEQ ID NO: 700 - RNA, 22 nt, synthetic construct
  source 1..22: mol_type = other RNA
agtatacttacaggctccaata

SEQ ID NO: 701 - RNA, 22 nt, synthetic construct
  source 1..22: mol_type = other RNA
caggctccaatagtggtcagtc

SEQ ID NO: 702 - RNA, 22 nt, synthetic construct
  source 1..22: mol_type = other RNA
caatagtggtcagtccaggagc

SEQ ID NO: 703 - RNA, 22 nt, synthetic construct
  source 1..22: mol_type = other RNA
gtggtcagtccaggagctaggt

SEQ ID NO: 704 - RNA, 22 nt, synthetic construct
  source 1..22: mol_type = other RNA
ttgccctcagctcttgaagtaa

SEQ ID NO: 705 - RNA, 22 nt, synthetic construct
  source 1..22: mol_type = other RNA
agtatactggatcccattctct

SEQ ID NO: 706 - RNA, 22 nt, synthetic construct
  source 1..22: mol_type = other RNA
actattggagcctgtaagtata

SEQ ID NO: 707 - RNA, 22 nt, synthetic construct
  source 1..22: mol_type = other RNA
ctagctcctggactgaccacta

SEQ ID NO: 708 - RNA, 22 nt, synthetic construct
  source 1..22: mol_type = other RNA
gcaaagcagcctgacctagctc

SEQ ID NO: 709 - RNA, 22 nt, synthetic construct
  source 1..22: mol_type = other RNA
aaaccgtttacttcaagagctg

SEQ ID NO: 710 - RNA, 22 nt, synthetic construct
  source 1..22: mol_type = other RNA
taaaccgtttacttcaagagct

SEQ ID NO: 711 - RNA, 22 nt, synthetic construct
  source 1..22: mol_type = other RNA
agatctgagctctgagtggaag

SEQ ID NO: 712 - RNA, 22 nt, synthetic construct
  source 1..22: mol_type = other RNA
agaagatctgagctctgagtgg

SEQ ID NO: 713 - RNA, 22 nt, synthetic construct
  source 1..22: mol_type = other RNA
agttagaagatctgagctctga

SEQ ID NO: 714 - RNA, 22 nt, synthetic construct
  source 1..22: mol_type = other RNA
atgtgtatgcttttctgttaaa

SEQ ID NO: 715 - RNA, 21 nt, synthetic construct
  source 1..21: mol_type = other RNA
atgggatccagtatacttaca

SEQ ID NO: 716 - RNA, 20 nt, synthetic construct
  source 1..20: mol_type = other RNA
atgggatccagtatacttac

SEQ ID NO: 717 - RNA, 19 nt, synthetic construct
  source 1..19: mol_type = other RNA
tgggatccagtatacttac

SEQ ID NO: 718 - RNA, 19 nt, synthetic construct
  source 1..19: mol_type = other RNA
atgggatccagtatactta

SEQ ID NO: 719 - RNA, 19 nt, synthetic construct
  source 1..19: mol_type = other RNA
gatccagtatacttacagg

SEQ ID NO: 720 - RNA, 19 nt, synthetic construct
  source 1..19: mol_type = other RNA
ggatccagtatacttacag

SEQ ID NO: 721 - RNA, 19 nt, synthetic construct
  source 1..19: mol_type = other RNA
aatgggatccagtatactt

SEQ ID NO: 722 - RNA, 25 nt, synthetic construct
  source 1..25: mol_type = other RNA
gtacctccaacatcaaggaagatgg

SEQ ID NO: 723 - RNA, 25 nt, synthetic construct
  source 1..25: mol_type = other RNA
tacctccaacatcaaggaagatggc

SEQ ID NO: 724 - RNA, 25 nt, synthetic construct
  source 1..25: mol_type = other RNA
acctccaacatcaaggaagatggca

SEQ ID NO: 725 - RNA, 25 nt, synthetic construct
  source 1..25: mol_type = other RNA
cctccaacatcaaggaagatggcat

SEQ ID NO: 726 - RNA, 25 nt, synthetic construct
  source 1..25: mol_type = other RNA
ctccaacatcaaggaagatggcatt

SEQ ID NO: 727 - RNA, 25 nt, synthetic construct
  source 1..25: mol_type = other RNA
tccaacatcaaggaagatggcattt

SEQ ID NO: 728 - RNA, 25 nt, synthetic construct
  source 1..25: mol_type = other RNA
ccaacatcaaggaagatggcatttc

SEQ ID NO: 729 - RNA, 25 nt, synthetic construct
  source 1..25: mol_type = other RNA
caacatcaaggaagatggcatttct

SEQ ID NO: 730 - RNA, 25 nt, synthetic construct
  source 1..25: mol_type = other RNA
aacatcaaggaagatggcatttcta

SEQ ID NO: 731 - RNA, 25 nt, synthetic construct
  source 1..25: mol_type = other RNA
acatcaaggaagatggcatttctag

SEQ ID NO: 732 - RNA, 25 nt, synthetic construct
  source 1..25: mol_type = other RNA
catcaaggaagatggcatttctagt

SEQ ID NO: 733 - RNA, 25 nt, synthetic construct
  source 1..25: mol_type = other RNA
atcaaggaagatggcatttctagtt

SEQ ID NO: 734 - RNA, 25 nt, synthetic construct
  source 1..25: mol_type = other RNA
tcaaggaagatggcatttctagttt

SEQ ID NO: 735 - RNA, 25 nt, synthetic construct
  source 1..25: mol_type = other RNA
caaggaagatggcatttctagtttg

SEQ ID NO: 736 - RNA, 25 nt, synthetic construct
  source 1..25: mol_type = other RNA
aaggaagatggcatttctagtttgg

SEQ ID NO: 737 - RNA, 25 nt, synthetic construct
  source 1..25: mol_type = other RNA
aggaagatggcatttctagtttgga

SEQ ID NO: 738 - RNA, 25 nt, synthetic construct
  source 1..25: mol_type = other RNA
ggaagatggcatttctagtttggag

SEQ ID NO: 739 - RNA, 25 nt, synthetic construct
  source 1..25: mol_type = other RNA
gaagatggcatttctagtttggaga

SEQ ID NO: 740 - RNA, 25 nt, synthetic construct
  source 1..25: mol_type = other RNA
aagatggcatttctagtttggagat

SEQ ID NO: 741 - RNA, 25 nt, synthetic construct
  source 1..25: mol_type = other RNA
agatggcatttctagtttggagatg

SEQ ID NO: 742 - RNA, 25 nt, synthetic construct
  source 1..25: mol_type = other RNA
gatggcatttctagtttggagatgg

SEQ ID NO: 743 - RNA, 25 nt, synthetic construct
  source 1..25: mol_type = other RNA
atggcatttctagtttggagatggc

SEQ ID NO: 744 - RNA, 25 nt, synthetic construct
  source 1..25: mol_type = other RNA
tggcatttctagtttggagatggca

SEQ ID NO: 745 - RNA, 25 nt, synthetic construct
  source 1..25: mol_type = other RNA
ggcatttctagtttggagatggcag

SEQ ID NO: 746 - RNA, 25 nt, synthetic construct
  source 1..25: mol_type = other RNA
gcatttctagtttggagatggcagt

SEQ ID NO: 747 - RNA, 25 nt, synthetic construct
  source 1..25: mol_type = other RNA
catttctagtttggagatggcagtt

SEQ ID NO: 748 - RNA, 25 nt, synthetic construct
  source 1..25: mol_type = other RNA
atttctagtttggagatggcagttt

SEQ ID NO: 749 - RNA, 25 nt, synthetic construct
  source 1..25: mol_type = other RNA
tttctagtttggagatggcagtttc

SEQ ID NO: 750 - RNA, 25 nt, synthetic construct
  source 1..25: mol_type = other RNA
ttctagtttggagatggcagtttcc

SEQ ID NO: 751 - RNA, 25 nt, synthetic construct
  source 1..25: mol_type = other RNA
tctagtttggagatggcagtttcct

SEQ ID NO: 752 - RNA, 25 nt, synthetic construct
  source 1..25: mol_type = other RNA
ctagtttggagatggcagtttcctt

SEQ ID NO: 753 - RNA, 25 nt, synthetic construct
  source 1..25: mol_type = other RNA
tagtttggagatggcagtttcctta

SEQ ID NO: 754 - RNA, 25 nt, synthetic construct
  source 1..25: mol_type = other RNA
agtttggagatggcagtttccttag

SEQ ID NO: 755 - RNA, 25 nt, synthetic construct
  source 1..25: mol_type = other RNA
gtttggagatggcagtttccttagt

SEQ ID NO: 756 - RNA, 25 nt, synthetic construct
  source 1..25: mol_type = other RNA
tttggagatggcagtttccttagta

SEQ ID NO: 757 - RNA, 25 nt, synthetic construct
  source 1..25: mol_type = other RNA
ttggagatggcagtttccttagtaa

SEQ ID NO: 758 - RNA, 25 nt, synthetic construct
  source 1..25: mol_type = other RNA
tggagatggcagtttccttagtaac

SEQ ID NO: 759 - RNA, 25 nt, synthetic construct
  source 1..25: mol_type = other RNA
gagatggcagtttccttagtaacca

SEQ ID NO: 760 - RNA, 25 nt, synthetic construct
  source 1..25: mol_type = other RNA
agatggcagtttccttagtaaccac

SEQ ID NO: 761 - RNA, 25 nt, synthetic construct
  source 1..25: mol_type = other RNA
gatggcagtttccttagtaaccaca

SEQ ID NO: 762 - RNA, 25 nt, synthetic construct
  source 1..25: mol_type = other RNA
atggcagtttccttagtaaccacag

SEQ ID NO: 763 - RNA, 25 nt, synthetic construct
  source 1..25: mol_type = other RNA
tggcagtttccttagtaaccacagg

SEQ ID NO: 764 - RNA, 25 nt, synthetic construct
  source 1..25: mol_type = other RNA
ggcagtttccttagtaaccacaggt

SEQ ID NO: 765 - RNA, 25 nt, synthetic construct
  source 1..25: mol_type = other RNA
gcagtttccttagtaaccacaggtt

SEQ ID NO: 766 - RNA, 25 nt, synthetic construct
  source 1..25: mol_type = other RNA
cagtttccttagtaaccacaggttg

SEQ ID NO: 767 - RNA, 25 nt, synthetic construct
  source 1..25: mol_type = other RNA
agtttccttagtaaccacaggttgt

SEQ ID NO: 768 - RNA, 25 nt, synthetic construct
  source 1..25: mol_type = other RNA
gtttccttagtaaccacaggttgtg

SEQ ID NO: 769 - RNA, 25 nt, synthetic construct
  source 1..25: mol_type = other RNA
tttccttagtaaccacaggttgtgt

SEQ ID NO: 770 - RNA, 25 nt, synthetic construct
  source 1..25: mol_type = other RNA
ttccttagtaaccacaggttgtgtc

SEQ ID NO: 771 - RNA, 25 nt, synthetic construct
  source 1..25: mol_type = other RNA
tccttagtaaccacaggttgtgtca

SEQ ID NO: 772 - RNA, 25 nt, synthetic construct
  source 1..25: mol_type = other RNA
ccttagtaaccacaggttgtgtcac

SEQ ID NO: 773 - RNA, 25 nt, synthetic construct
  source 1..25: mol_type = other RNA
cttagtaaccacaggttgtgtcacc

SEQ ID NO: 774 - RNA, 25 nt, synthetic construct
  source 1..25: mol_type = other RNA
ttagtaaccacaggttgtgtcacca

SEQ ID NO: 775 - RNA, 25 nt, synthetic construct
  source 1..25: mol_type = other RNA
tagtaaccacaggttgtgtcaccag

SEQ ID NO: 776 - RNA, 25 nt, synthetic construct
  source 1..25: mol_type = other RNA
agtaaccacaggttgtgtcaccaga

SEQ ID NO: 777 - RNA, 25 nt, synthetic construct
  source 1..25: mol_type = other RNA
gtaaccacaggttgtgtcaccagag

SEQ ID NO: 778 - RNA, 25 nt, synthetic construct
  source 1..25: mol_type = other RNA
taaccacaggttgtgtcaccagagt

SEQ ID NO: 779 - RNA, 25 nt, synthetic construct
  source 1..25: mol_type = other RNA
aaccacaggttgtgtcaccagagta

SEQ ID NO: 780 - RNA, 25 nt, synthetic construct
  source 1..25: mol_type = other RNA
accacaggttgtgtcaccagagtaa

SEQ ID NO: 781 - RNA, 25 nt, synthetic construct
  source 1..25: mol_type = other RNA
ccacaggttgtgtcaccagagtaac

SEQ ID NO: 782 - RNA, 25 nt, synthetic construct
  source 1..25: mol_type = other RNA
cacaggttgtgtcaccagagtaaca

SEQ ID NO: 783 - RNA, 25 nt, synthetic construct
  source 1..25: mol_type = other RNA
acaggttgtgtcaccagagtaacag

SEQ ID NO: 784 - RNA, 25 nt, synthetic construct
  source 1..25: mol_type = other RNA
caggttgtgtcaccagagtaacagt

SEQ ID NO: 785 - RNA, 25 nt, synthetic construct
  source 1..25: mol_type = other RNA
aggttgtgtcaccagagtaacagtc

SEQ ID NO: 786 - RNA, 25 nt, synthetic construct
  source 1..25: mol_type = other RNA
ggttgtgtcaccagagtaacagtct

SEQ ID NO: 787 - RNA, 25 nt, synthetic construct
  source 1..25: mol_type = other RNA
gttgtgtcaccagagtaacagtctg

SEQ ID NO: 788 - RNA, 25 nt, synthetic construct
  source 1..25: mol_type = other RNA
ttgtgtcaccagagtaacagtctga

SEQ ID NO: 789 - RNA, 25 nt, synthetic construct
  source 1..25: mol_type = other RNA
tgtgtcaccagagtaacagtctgag

SEQ ID NO: 790 - RNA, 25 nt, synthetic construct
  source 1..25: mol_type = other RNA
gtgtcaccagagtaacagtctgagt

SEQ ID NO: 791 - RNA, 25 nt, synthetic construct
  source 1..25: mol_type = other RNA
tgtcaccagagtaacagtctgagta

SEQ ID NO: 792 - RNA, 25 nt, synthetic construct
  source 1..25: mol_type = other RNA
gtcaccagagtaacagtctgagtag

SEQ ID NO: 793 - RNA, 25 nt, synthetic construct
  source 1..25: mol_type = other RNA
tcaccagagtaacagtctgagtagg

SEQ ID NO: 794 - RNA, 25 nt, synthetic construct
  source 1..25: mol_type = other RNA
caccagagtaacagtctgagtagga

SEQ ID NO: 795 - RNA, 25 nt, synthetic construct
  source 1..25: mol_type = other RNA
accagagtaacagtctgagtaggag

SEQ ID NO: 796 - RNA, 22 nt, synthetic construct
  source 1..22: mol_type = other RNA
tagtaaccacaggttgtgtcac

SEQ ID NO: 797 - RNA, 22 nt, synthetic construct
  source 1..22: mol_type = other RNA
gttgtgtcaccagagtaacagt

SEQ ID NO: 798 - RNA, 22 nt, synthetic construct
  source 1..22: mol_type = other RNA
tctgagtaggagctaaaatatt

SEQ ID NO: 799 - RNA, 22 nt, synthetic construct
  source 1..22: mol_type = other RNA
gagggtgatggtgggtgacctt

SEQ ID NO: 800 - RNA, 22 nt, synthetic construct
  source 1..22: mol_type = other RNA
tataaaatcacagagggtgatg

SEQ ID NO: 801 - RNA, 22 nt, synthetic construct
  source 1..22: mol_type = other RNA
ttgatcaagttataaaatcaca

SEQ ID NO: 802 - RNA, 22 nt, synthetic construct
  source 1..22: mol_type = other RNA
tgatcatctcgttgatatcctc

SEQ ID NO: 803 - RNA, 22 nt, synthetic construct
  source 1..22: mol_type = other RNA
ttgatcaagcagagaaagccag

SEQ ID NO: 804 - RNA, 22 nt, synthetic construct
  source 1..22: mol_type = other RNA
agtcggtaagttctgtccaagc

SEQ ID NO: 805 - RNA, 22 nt, synthetic construct
  source 1..22: mol_type = other RNA
gcccggttgaaatctgccagag

SEQ ID NO: 806 - RNA, 22 nt, synthetic construct
  source 1..22: mol_type = other RNA
cagagcaggtacctccaacatc

SEQ ID NO: 807 - RNA, 22 nt, synthetic construct
  source 1..22: mol_type = other RNA
ggtacctccaacatcaaggaag

SEQ ID NO: 808 - RNA, 22 nt, synthetic construct
  source 1..22: mol_type = other RNA
caaggaagatggcatttctagt

SEQ ID NO: 809 - RNA, 22 nt, synthetic construct
  source 1..22: mol_type = other RNA
agatggcatttctagtttggag

SEQ ID NO: 810 - RNA, 22 nt, synthetic construct
  source 1..22: mol_type = other RNA
atggcagtttccttagtaacca

SEQ ID NO: 811 - RNA, 22 nt, synthetic construct
  source 1..22: mol_type = other RNA
gtcaccagagtaacagtctgag

SEQ ID NO: 812 - RNA, 22 nt, synthetic construct
  source 1..22: mol_type = other RNA
tctgagtaggagctaaaatatt

SEQ ID NO: 813 - RNA, 22 nt, synthetic construct
  source 1..22: mol_type = other RNA
aaatattttgggtttttgcaaa

SEQ ID NO: 814 - RNA, 22 nt, synthetic construct
  source 1..22: mol_type = other RNA
gtatgagaaaaaatgataaaag

SEQ ID NO: 815 - RNA, 22 nt, synthetic construct
  source 1..22: mol_type = other RNA
caacgagatgatcatcaagcag

SEQ ID NO: 816 - RNA, 22 nt, synthetic construct
  source 1..22: mol_type = other RNA
gagggtgatggtgggtgacctt

SEQ ID NO: 817 - RNA, 22 nt, synthetic construct
  source 1..22: mol_type = other RNA
ataaaatcacagagggtgatgg

SEQ ID NO: 818 - RNA, 22 nt, synthetic construct
  source 1..22: mol_type = other RNA
tataaaatcacagagggtgatg

SEQ ID NO: 819 - RNA, 22 nt, synthetic construct
  source 1..22: mol_type = other RNA
agttataaaatcacagagggtg

SEQ ID NO: 820 - RNA, 22 nt, synthetic construct
  source 1..22: mol_type = other RNA
tgatcaagttataaaatcacag

SEQ ID NO: 821 - RNA, 22 nt, synthetic construct
  source 1..22: mol_type = other RNA
ctgagtaggagctaaaatattt

SEQ ID NO: 822 - DNA, 22 bp, synthetic construct
  source 1..22: mol_type = other DNA
ttgatcaagttataaaatcaca

SEQ ID NO: 823 - DNA, 22 bp, synthetic construct
  source 1..22: mol_type = other DNA
gggcttggacagaacttaccga

SEQ ID NO: 824 - DNA, 22 bp, synthetic construct
  source 1..22: mol_type = other DNA
ctctggcagatttcaaccgggc

SEQ ID NO: 825 - DNA, 22 bp, synthetic construct
  source 1..22: mol_type = other DNA
acctgctctggcagatttcaac

SEQ ID NO: 826 - DNA, 22 bp, synthetic construct
  source 1..22: mol_type = other DNA
tacctgctctggcagatttcaa

SEQ ID NO: 827 - DNA, 22 bp, synthetic construct
  source 1..22: mol_type = other DNA
cttgatgttggaggtacctgct

SEQ ID NO: 828 - DNA, 22 bp, synthetic construct
  source 1..22: mol_type = other DNA
aatgccatcttccttgatgttg

SEQ ID NO: 829 - DNA, 22 bp, synthetic construct
  source 1..22: mol_type = other DNA
agaaatgccatcttccttgatg

SEQ ID NO: 830 - DNA, 22 bp, synthetic construct
  source 1..22: mol_type = other DNA
ggtgacacaacctgtggttact

SEQ ID NO: 831 - DNA, 22 bp, synthetic construct
  source 1..22: mol_type = other DNA
tgttactctggtgacacaacct

SEQ ID NO: 832 - DNA, 22 bp, synthetic construct
  source 1..22: mol_type = other DNA
agctcctactcagactgttact

SEQ ID NO: 833 - DNA, 30 bp, synthetic construct
  source 1..30: mol_type = other DNA
  modified_base order(8,13): mod_base = i
cggtaagntctgncctcaaggaagatggca

SEQ ID NO: 834 - DNA, 30 bp, synthetic construct
  source 1..30: mol_type = other DNA
ctcataccttctgcttcaaggaagatggca

SEQ ID NO: 835 - DNA, 30 bp, synthetic construct
  source 1..30: mol_type = other DNA
ctccaacatcaaggaagatggcatttctag

SEQ ID NO: 836 - DNA, 21 bp, synthetic construct
  source 1..21: mol_type = other DNA
aacatcaaggaagatggcatt

SEQ ID NO: 837 - DNA, 21 bp, synthetic construct
  source 1..21: mol_type = other DNA
tccaacatcaaggaagatggc

SEQ ID NO: 838 - DNA, 21 bp, synthetic construct
  source 1..21: mol_type = other DNA
acctccaacatcaaggaagat

SEQ ID NO: 839 - RNA, 21 nt, synthetic construct
  source 1..21: mol_type = other RNA
gagtaacagtctgagtaggag

SEQ ID NO: 840 - RNA, 21 nt, synthetic construct
  source 1..21: mol_type = other RNA
tgtgtcaccagagtaacagtc

SEQ ID NO: 841 - RNA, 21 nt, synthetic construct
  source 1..21: mol_type = other RNA
aaccacaggttgtgtcaccag

SEQ ID NO: 842 - RNA, 21 nt, synthetic construct
  source 1..21: mol_type = other RNA
tttccttagtaaccacaggtt

SEQ ID NO: 843 - RNA, 21 nt, synthetic construct
  source 1..21: mol_type = other RNA
gagatggcagtttccttagta

SEQ ID NO: 844 - RNA, 21 nt, synthetic construct
  source 1..21: mol_type = other RNA
ttctagtttggagatggcagt

SEQ ID NO: 845 - RNA, 21 nt, synthetic construct
  source 1..21: mol_type = other RNA
aagatggcatttctagtttgg

SEQ ID NO: 846 - RNA, 21 nt, synthetic construct
  source 1..21: mol_type = other RNA
aacatcaaggaagatggcatt

SEQ ID NO: 847 - RNA, 21 nt, synthetic construct
  source 1..21: mol_type = other RNA
aggtacctccaacatcaagga

SEQ ID NO: 848 - RNA, 21 nt, synthetic construct
  source 1..21: mol_type = other RNA
ctgccagagcaggtacctcca

SEQ ID NO: 849 - RNA, 21 nt, synthetic construct
  source 1..21: mol_type = other RNA
cggttgaaatctgccagagca

SEQ ID NO: 850 - RNA, 21 nt, synthetic construct
  source 1..21: mol_type = other RNA
tgtccaagcccggttgaaatc

SEQ ID NO: 851 - RNA, 21 nt, synthetic construct
  source 1..21: mol_type = other RNA
cggtaagttctgtccaagccc

SEQ ID NO: 852 - RNA, 21 nt, synthetic construct
  source 1..21: mol_type = other RNA
gaaagccagtcggtaagttct

SEQ ID NO: 853 - RNA, 21 nt, synthetic construct
  source 1..21: mol_type = other RNA
atcaagcagagaaagccagtc

SEQ ID NO: 854 - RNA, 21 nt, synthetic construct
  source 1..21: mol_type = other RNA
ttataacttgatcaagcagag

SEQ ID NO: 855 - RNA, 21 nt, synthetic construct
  source 1..21: mol_type = other RNA
ctctgtgattttataacttga

SEQ ID NO: 856 - RNA, 21 nt, synthetic construct
  source 1..21: mol_type = other RNA
caccatcaccctctgtgattt

SEQ ID NO: 857 - RNA, 21 nt, synthetic construct
  source 1..21: mol_type = other RNA
caaggtcacccaccatcaccc

SEQ ID NO: 858 - RNA, 21 nt, synthetic construct
  source 1..21: mol_type = other RNA
ttgatatcctcaaggtcaccc

SEQ ID NO: 859 - RNA, 21 nt, synthetic construct
  source 1..21: mol_type = other RNA
gatcatctcgttgatatcctc

SEQ ID NO: 860 - RNA, 21 nt, synthetic construct
  source 1..21: mol_type = other RNA
tctgcttgatgatcatctcgt

SEQ ID NO: 861 - RNA, 21 nt, synthetic construct
  source 1..21: mol_type = other RNA
ggcatttctagtttggagatg

SEQ ID NO: 862 - RNA, 21 nt, synthetic construct
  source 1..21: mol_type = other RNA
caaggaagatggcatttctag

SEQ ID NO: 863 - RNA, 21 nt, synthetic construct
  source 1..21: mol_type = other RNA
cctccaacatcaaggaagatg

SEQ ID NO: 864 - RNA, 25 nt, synthetic construct
  source 1..25: mol_type = other RNA
agcctcttgattgctggtcttgttt

SEQ ID NO: 865 - RNA, 25 nt, synthetic construct
  source 1..25: mol_type = other RNA
gcctcttgattgctggtcttgtttt

SEQ ID NO: 866 - RNA, 25 nt, synthetic construct
  source 1..25: mol_type = other RNA
cctcttgattgctggtcttgttttt

SEQ ID NO: 867 - RNA, 20 nt, synthetic construct
  source 1..20: mol_type = other RNA
cctcttgattgctggtcttg

SEQ ID NO: 868 - RNA, 25 nt, synthetic construct
  source 1..25: mol_type = other RNA
ctcttgattgctggtcttgtttttc

SEQ ID NO: 869 - RNA, 25 nt, synthetic construct
  source 1..25: mol_type = other RNA
tcttgattgctggtcttgtttttca

SEQ ID NO: 870 - RNA, 25 nt, synthetic construct
  source 1..25: mol_type = other RNA
cttgattgctggtcttgtttttcaa

SEQ ID NO: 871 - RNA, 25 nt, synthetic construct
  source 1..25: mol_type = other RNA
ttgattgctggtcttgtttttcaaa

SEQ ID NO: 872 - RNA, 25 nt, synthetic construct
  source 1..25: mol_type = other RNA
tgattgctggtcttgtttttcaaat

SEQ ID NO: 873 - RNA, 25 nt, synthetic construct
  source 1..25: mol_type = other RNA
gattgctggtcttgtttttcaaatt

SEQ ID NO: 874 - RNA, 20 nt, synthetic construct
  source 1..20: mol_type = other RNA
gattgctggtcttgtttttc

SEQ ID NO: 875 - RNA, 25 nt, synthetic construct
  source 1..25: mol_type = other RNA
attgctggtcttgtttttcaaattt

SEQ ID NO: 876 - RNA, 25 nt, synthetic construct
  source 1..25: mol_type = other RNA
ttgctggtcttgtttttcaaatttt

SEQ ID NO: 877 - RNA, 25 nt, synthetic construct
  source 1..25: mol_type = other RNA
tgctggtcttgtttttcaaattttg

SEQ ID NO: 878 - RNA, 25 nt, synthetic construct
  source 1..25: mol_type = other RNA
gctggtcttgtttttcaaattttgg

SEQ ID NO: 879 - RNA, 25 nt, synthetic construct
  source 1..25: mol_type = other RNA
ctggtcttgtttttcaaattttggg

SEQ ID NO: 880 - RNA, 25 nt, synthetic construct
  source 1..25: mol_type = other RNA
tggtcttgtttttcaaattttgggc

SEQ ID NO: 881 - RNA, 25 nt, synthetic construct
  source 1..25: mol_type = other RNA
ggtcttgtttttcaaattttgggca

SEQ ID NO: 882 - RNA, 25 nt, synthetic construct
  source 1..25: mol_type = other RNA
gtcttgtttttcaaattttgggcag

SEQ ID NO: 883 - RNA, 25 nt, synthetic construct
  source 1..25: mol_type = other RNA
tcttgtttttcaaattttgggcagc

SEQ ID NO: 884 - RNA, 25 nt, synthetic construct
  source 1..25: mol_type = other RNA
cttgtttttcaaattttgggcagcg

SEQ ID NO: 885 - RNA, 25 nt, synthetic construct
  source 1..25: mol_type = other RNA
ttgtttttcaaattttgggcagcgg

SEQ ID NO: 886 - RNA, 25 nt, synthetic construct
  source 1..25: mol_type = other RNA
tgtttttcaaattttgggcagcggt

SEQ ID NO: 887 - RNA, 25 nt, synthetic construct
  source 1..25: mol_type = other RNA
gtttttcaaattttgggcagcggta

SEQ ID NO: 888 - RNA, 25 nt, synthetic construct
  source 1..25: mol_type = other RNA
tttttcaaattttgggcagcggtaa

SEQ ID NO: 889 - RNA, 25 nt, synthetic construct
  source 1..25: mol_type = other RNA
ttttcaaattttgggcagcggtaat

SEQ ID NO: 890 - RNA, 25 nt, synthetic construct
  source 1..25: mol_type = other RNA
tttcaaattttgggcagcggtaatg

SEQ ID NO: 891 - RNA, 25 nt, synthetic construct
  source 1..25: mol_type = other RNA
ttcaaattttgggcagcggtaatga

SEQ ID NO: 892 - RNA, 25 nt, synthetic construct
  source 1..25: mol_type = other RNA
tcaaattttgggcagcggtaatgag

SEQ ID NO: 893 - RNA, 25 nt, synthetic construct
  source 1..25: mol_type = other RNA
caaattttgggcagcggtaatgagt

SEQ ID NO: 894 - RNA, 25 nt, synthetic construct
  source 1..25: mol_type = other RNA
aaattttgggcagcggtaatgagtt

SEQ ID NO: 895 - RNA, 25 nt, synthetic construct
  source 1..25: mol_type = other RNA
aattttgggcagcggtaatgagttc

SEQ ID NO: 896 - RNA, 25 nt, synthetic construct
  source 1..25: mol_type = other RNA
attttgggcagcggtaatgagttct

SEQ ID NO: 897 - RNA, 25 nt, synthetic construct
  source 1..25: mol_type = other RNA
ttttgggcagcggtaatgagttctt

SEQ ID NO: 898 - RNA, 25 nt, synthetic construct
  source 1..25: mol_type = other RNA
tttgggcagcggtaatgagttcttc

SEQ ID NO: 899 - RNA, 25 nt, synthetic construct
  source 1..25: mol_type = other RNA
ttgggcagcggtaatgagttcttcc

SEQ ID NO: 900 - RNA, 25 nt, synthetic construct
  source 1..25: mol_type = other RNA
tgggcagcggtaatgagttcttcca

SEQ ID NO: 901 - RNA, 25 nt, synthetic construct
  source 1..25: mol_type = other RNA
gggcagcggtaatgagttcttccaa

SEQ ID NO: 902 - RNA, 25 nt, synthetic construct
  source 1..25: mol_type = other RNA
ggcagcggtaatgagttcttccaac

SEQ ID NO: 903 - RNA, 25 nt, synthetic construct
  source 1..25: mol_type = other RNA
gcagcggtaatgagttcttccaact

SEQ ID NO: 904 - RNA, 25 nt, synthetic construct
  source 1..25: mol_type = other RNA
cagcggtaatgagttcttccaactg

SEQ ID NO: 905 - RNA, 25 nt, synthetic construct
  source 1..25: mol_type = other RNA
agcggtaatgagttcttccaactgg

SEQ ID NO: 906 - RNA, 25 nt, synthetic construct
  source 1..25: mol_type = other RNA
gcggtaatgagttcttccaactggg

SEQ ID NO: 907 - RNA, 25 nt, synthetic construct
  source 1..25: mol_type = other RNA
cggtaatgagttcttccaactgggg

SEQ ID NO: 908 - RNA, 25 nt, synthetic construct
  source 1..25: mol_type = other RNA
ggtaatgagttcttccaactgggga

SEQ ID NO: 909 - RNA, 22 nt, synthetic construct
  source 1..22: mol_type = other RNA
ggtaatgagttcttccaactgg

SEQ ID NO: 910 - RNA, 25 nt, synthetic construct
  source 1..25: mol_type = other RNA
gtaatgagttcttccaactggggac

SEQ ID NO: 911 - RNA, 25 nt, synthetic construct
  source 1..25: mol_type = other RNA
taatgagttcttccaactggggacg

SEQ ID NO: 912 - RNA, 25 nt, synthetic construct
  source 1..25: mol_type = other RNA
aatgagttcttccaactggggacgc

SEQ ID NO: 913 - RNA, 25 nt, synthetic construct
  source 1..25: mol_type = other RNA
atgagttcttccaactggggacgcc

SEQ ID NO: 914 - RNA, 25 nt, synthetic construct
  source 1..25: mol_type = other RNA
tgagttcttccaactggggacgcct

SEQ ID NO: 915 - RNA, 25 nt, synthetic construct
  source 1..25: mol_type = other RNA
gagttcttccaactggggacgcctc

SEQ ID NO: 916 - RNA, 25 nt, synthetic construct
  source 1..25: mol_type = other RNA
agttcttccaactggggacgcctct

SEQ ID NO: 917 - RNA, 25 nt, synthetic construct
  source 1..25: mol_type = other RNA
gttcttccaactggggacgcctctg

SEQ ID NO: 918 - RNA, 25 nt, synthetic construct
  source 1..25: mol_type = other RNA
ttcttccaactggggacgcctctgt

SEQ ID NO: 919 - RNA, 25 nt, synthetic construct
  source 1..25: mol_type = other RNA
tcttccaactggggacgcctctgtt

SEQ ID NO: 920 - RNA, 25 nt, synthetic construct
  source 1..25: mol_type = other RNA
cttccaactggggacgcctctgttc

SEQ ID NO: 921 - RNA, 25 nt, synthetic construct
  source 1..25: mol_type = other RNA
ttccaactggggacgcctctgttcc

SEQ ID NO: 922 - RNA, 25 nt, synthetic construct
  source 1..25: mol_type = other RNA
tccaactggggacgcctctgttcca

SEQ ID NO: 923 - RNA, 25 nt, synthetic construct
  source 1..25: mol_type = other RNA
ccaactggggacgcctctgttccaa

SEQ ID NO: 924 - RNA, 25 nt, synthetic construct
  source 1..25: mol_type = other RNA
caactggggacgcctctgttccaaa

SEQ ID NO: 925 - RNA, 25 nt, synthetic construct
  source 1..25: mol_type = other RNA
aactggggacgcctctgttccaaat

SEQ ID NO: 926 - RNA, 25 nt, synthetic construct
  source 1..25: mol_type = other RNA
actggggacgcctctgttccaaatc

SEQ ID NO: 927 - RNA, 25 nt, synthetic construct
  source 1..25: mol_type = other RNA
ctggggacgcctctgttccaaatcc

SEQ ID NO: 928 - RNA, 25 nt, synthetic construct
  source 1..25: mol_type = other RNA
tggggacgcctctgttccaaatcct

SEQ ID NO: 929 - RNA, 25 nt, synthetic construct
  source 1..25: mol_type = other RNA
ggggacgcctctgttccaaatcctg

SEQ ID NO: 930 - RNA, 25 nt, synthetic construct
  source 1..25: mol_type = other RNA
gggacgcctctgttccaaatcctgc

SEQ ID NO: 931 - RNA, 25 nt, synthetic construct
  source 1..25: mol_type = other RNA
ggacgcctctgttccaaatcctgca

SEQ ID NO: 932 - RNA, 25 nt, synthetic construct
  source 1..25: mol_type = other RNA
gacgcctctgttccaaatcctgcat

SEQ ID NO: 933 - RNA, 25 nt, synthetic construct
  source 1..25: mol_type = other RNA
ctctggcctgtcctaagacctgctc

SEQ ID NO: 934 - RNA, 25 nt, synthetic construct
  source 1..25: mol_type = other RNA
tctggcctgtcctaagacctgctca

SEQ ID NO: 935 - RNA, 25 nt, synthetic construct
  source 1..25: mol_type = other RNA
ctggcctgtcctaagacctgctcag

SEQ ID NO: 936 - RNA, 25 nt, synthetic construct
  source 1..25: mol_type = other RNA
tggcctgtcctaagacctgctcagc

SEQ ID NO: 937 - RNA, 25 nt, synthetic construct
  source 1..25: mol_type = other RNA
ggcctgtcctaagacctgctcagct

SEQ ID NO: 938 - RNA, 25 nt, synthetic construct
  source 1..25: mol_type = other RNA
gcctgtcctaagacctgctcagctt

SEQ ID NO: 939 - RNA, 25 nt, synthetic construct
  source 1..25: mol_type = other RNA
cctgtcctaagacctgctcagcttc

SEQ ID NO: 940 - RNA, 25 nt, synthetic construct
  source 1..25: mol_type = other RNA
ctgtcctaagacctgctcagcttct

SEQ ID NO: 941 - RNA, 25 nt, synthetic construct
  source 1..25: mol_type = other RNA
tgtcctaagacctgctcagcttctt

SEQ ID NO: 942 - RNA, 25 nt, synthetic construct
  source 1..25: mol_type = other RNA
gtcctaagacctgctcagcttcttc

SEQ ID NO: 943 - RNA, 25 nt, synthetic construct
  source 1..25: mol_type = other RNA
tcctaagacctgctcagcttcttcc

SEQ ID NO: 944 - RNA, 25 nt, synthetic construct
  source 1..25: mol_type = other RNA
cctaagacctgctcagcttcttcct

SEQ ID NO: 945 - RNA, 25 nt, synthetic construct
  source 1..25: mol_type = other RNA
ctaagacctgctcagcttcttcctt

SEQ ID NO: 946 - RNA, 25 nt, synthetic construct
  source 1..25: mol_type = other RNA
taagacctgctcagcttcttcctta

SEQ ID NO: 947 - RNA, 25 nt, synthetic construct
  source 1..25: mol_type = other RNA
aagacctgctcagcttcttccttag

SEQ ID NO: 948 - RNA, 25 nt, synthetic construct
  source 1..25: mol_type = other RNA
agacctgctcagcttcttccttagc

SEQ ID NO: 949 - RNA, 25 nt, synthetic construct
  source 1..25: mol_type = other RNA
gacctgctcagcttcttccttagct

SEQ ID NO: 950 - RNA, 25 nt, synthetic construct
  source 1..25: mol_type = other RNA
acctgctcagcttcttccttagctt

SEQ ID NO: 951 - RNA, 25 nt, synthetic construct
  source 1..25: mol_type = other RNA
cctgctcagcttcttccttagcttc

SEQ ID NO: 952 - RNA, 25 nt, synthetic construct
  source 1..25: mol_type = other RNA
ctgctcagcttcttccttagcttcc

SEQ ID NO: 953 - RNA, 25 nt, synthetic construct
  source 1..25: mol_type = other RNA
tgctcagcttcttccttagcttcca

SEQ ID NO: 954 - RNA, 25 nt, synthetic construct
  source 1..25: mol_type = other RNA
gctcagcttcttccttagcttccag

SEQ ID NO: 955 - RNA, 25 nt, synthetic construct
  source 1..25: mol_type = other RNA
ctcagcttcttccttagcttccagc

SEQ ID NO: 956 - RNA, 25 nt, synthetic construct
  source 1..25: mol_type = other RNA
tcagcttcttccttagcttccagcc

SEQ ID NO: 957 - RNA, 25 nt, synthetic construct
  source 1..25: mol_type = other RNA
cagcttcttccttagcttccagcca

SEQ ID NO: 958 - RNA, 25 nt, synthetic construct
  source 1..25: mol_type = other RNA
agcttcttccttagcttccagccat

SEQ ID NO: 959 - RNA, 25 nt, synthetic construct
  source 1..25: mol_type = other RNA
gcttcttccttagcttccagccatt

SEQ ID NO: 960 - RNA, 25 nt, synthetic construct
  source 1..25: mol_type = other RNA
cttcttccttagcttccagccattg

SEQ ID NO: 961 - RNA, 25 nt, synthetic construct
  source 1..25: mol_type = other RNA
ttcttccttagcttccagccattgt

SEQ ID NO: 962 - RNA, 25 nt, synthetic construct
  source 1..25: mol_type = other RNA
tcttccttagcttccagccattgtg

SEQ ID NO: 963 - RNA, 25 nt, synthetic construct
  source 1..25: mol_type = other RNA
cttccttagcttccagccattgtgt

SEQ ID NO: 964 - RNA, 25 nt, synthetic construct
  source 1..25: mol_type = other RNA
ttccttagcttccagccattgtgtt

SEQ ID NO: 965 - RNA, 25 nt, synthetic construct
  source 1..25: mol_type = other RNA
tccttagcttccagccattgtgttg

SEQ ID NO: 966 - RNA, 25 nt, synthetic construct
  source 1..25: mol_type = other RNA
ccttagcttccagccattgtgttga

SEQ ID NO: 967 - RNA, 25 nt, synthetic construct
  source 1..25: mol_type = other RNA
cttagcttccagccattgtgttgaa

SEQ ID NO: 968 - RNA, 25 nt, synthetic construct
  source 1..25: mol_type = other RNA
ttagcttccagccattgtgttgaat

SEQ ID NO: 969 - RNA, 25 nt, synthetic construct
  source 1..25: mol_type = other RNA
tagcttccagccattgtgttgaatc

SEQ ID NO: 970 - RNA, 25 nt, synthetic construct
  source 1..25: mol_type = other RNA
agcttccagccattgtgttgaatcc

SEQ ID NO: 971 - RNA, 25 nt, synthetic construct
  source 1..25: mol_type = other RNA
gcttccagccattgtgttgaatcct

SEQ ID NO: 972 - RNA, 25 nt, synthetic construct
  source 1..25: mol_type = other RNA
cttccagccattgtgttgaatcctt

SEQ ID NO: 973 - RNA, 25 nt, synthetic construct
  source 1..25: mol_type = other RNA
ttccagccattgtgttgaatccttt

SEQ ID NO: 974 - RNA, 25 nt, synthetic construct
  source 1..25: mol_type = other RNA
tccagccattgtgttgaatccttta

SEQ ID NO: 975 - RNA, 25 nt, synthetic construct
  source 1..25: mol_type = other RNA
ccagccattgtgttgaatcctttaa

SEQ ID NO: 976 - RNA, 25 nt, synthetic construct
  source 1..25: mol_type = other RNA
cagccattgtgttgaatcctttaac

SEQ ID NO: 977 - RNA, 25 nt, synthetic construct
  source 1..25: mol_type = other RNA
agccattgtgttgaatcctttaaca

SEQ ID NO: 978 - RNA, 25 nt, synthetic construct
  source 1..25: mol_type = other RNA
gccattgtgttgaatcctttaacat

SEQ ID NO: 979 - RNA, 25 nt, synthetic construct
  source 1..25: mol_type = other RNA
ccattgtgttgaatcctttaacatt

SEQ ID NO: 980 - RNA, 25 nt, synthetic construct
  source 1..25: mol_type = other RNA
cattgtgttgaatcctttaacattt

SEQ ID NO: 981 - RNA, 22 nt, synthetic construct
  source 1..22: mol_type = other RNA
ccttggtttctgtgattttctt

SEQ ID NO: 982 - RNA, 22 nt, synthetic construct
  source 1..22: mol_type = other RNA
tccttagcttccagccattgtg

SEQ ID NO: 983 - RNA, 22 nt, synthetic construct
  source 1..22: mol_type = other RNA
cttgtacttcatcccactgatt

SEQ ID NO: 984 - RNA, 22 nt, synthetic construct
  source 1..22: mol_type = other RNA
actgattctgaattctttcaac

SEQ ID NO: 985 - RNA, 22 nt, synthetic construct
  source 1..22: mol_type = other RNA
agccaagcttgagtcatggaag

SEQ ID NO: 986 - RNA, 22 nt, synthetic construct
  source 1..22: mol_type = other RNA
ttaggacaggccagagccaagc

SEQ ID NO: 987 - RNA, 22 nt, synthetic construct
  source 1..22: mol_type = other RNA
gcaacagttgaatgaaatgtta

SEQ ID NO: 988 - RNA, 22 nt, synthetic construct
  source 1..22: mol_type = other RNA
ccttcagaaccggaggcaacag

SEQ ID NO: 989 - RNA, 22 nt, synthetic construct
  source 1..22: mol_type = other RNA
agttgaaagaattcagaatcag

SEQ ID NO: 990 - RNA, 22 nt, synthetic construct
  source 1..22: mol_type = other RNA
ttttattctagttgaaagaatt

SEQ ID NO: 991 - RNA, 22 nt, synthetic construct
  source 1..22: mol_type = other RNA
tttttccttttattctagttga

SEQ ID NO: 992 - RNA, 22 nt, synthetic construct
  source 1..22: mol_type = other RNA
aaaggtatctttgatactaacc

SEQ ID NO: 993 - RNA, 22 nt, synthetic construct
  source 1..22: mol_type = other RNA
ccttggtttctgtgattttctt

SEQ ID NO: 994 - RNA, 22 nt, synthetic construct
  source 1..22: mol_type = other RNA
cttttggattgcatctactgta

SEQ ID NO: 995 - RNA, 22 nt, synthetic construct
  source 1..22: mol_type = other RNA
ttttggattgcatctactgtat

SEQ ID NO: 996 - RNA, 22 nt, synthetic construct
  source 1..22: mol_type = other RNA
accctccttccatgactcaagc

SEQ ID NO: 997 - RNA, 22 nt, synthetic construct
  source 1..22: mol_type = other RNA
cttccatgactcaagcttggct

SEQ ID NO: 998 - RNA, 22 nt, synthetic construct
  source 1..22: mol_type = other RNA
acatttcattcaactgttgcct

SEQ ID NO: 999 - RNA, 22 nt, synthetic construct
  source 1..22: mol_type = other RNA
tcaactgttgcctccggttctg

SEQ ID NO: 1000 - RNA, 22 nt, synthetic construct
  source 1..22: mol_type = other RNA
ccaaaagaaaatcacagaaacc

SEQ ID NO: 1001 - RNA, 22 nt, synthetic construct
  source 1..22: mol_type = other RNA
gccaagcttgagtcatggaagg

SEQ ID NO: 1002 - RNA, 22 nt, synthetic construct
  source 1..22: mol_type = other RNA
agccaagcttgagtcatggaag

SEQ ID NO: 1003 - RNA, 22 nt, synthetic construct
  source 1..22: mol_type = other RNA
cagagccaagcttgagtcatgg

SEQ ID NO: 1004 - RNA, 22 nt, synthetic construct
  source 1..22: mol_type = other RNA
aggccagagccaagcttgagtc

SEQ ID NO: 1005 - RNA, 22 nt, synthetic construct
  source 1..22: mol_type = other RNA
agaagctgagcaggtcttagga

SEQ ID NO: 1006 - RNA, 22 nt, synthetic construct
  source 1..22: mol_type = other RNA
aaggaagaagctgagcaggtct

SEQ ID NO: 1007 - RNA, 22 nt, synthetic construct
  source 1..22: mol_type = other RNA
ggaagctaaggaagaagctgag

SEQ ID NO: 1008 - RNA, 22 nt, synthetic construct
  source 1..22: mol_type = other RNA
ttcaacacaatggctggaagct

SEQ ID NO: 1009 - RNA, 22 nt, synthetic construct
  source 1..22: mol_type = other RNA
gttaaaggattcaacacaatgg

SEQ ID NO: 1010 - RNA, 22 nt, synthetic construct
  source 1..22: mol_type = other RNA
aaatgttaaaggattcaacaca

SEQ ID NO: 1011 - RNA, 22 nt, synthetic construct
  source 1..22: mol_type = other RNA
gcaacagttgaatgaaatgtta

SEQ ID NO: 1012 - RNA, 22 nt, synthetic construct
  source 1..22: mol_type = other RNA
tacaagaacaccttcagaaccg

SEQ ID NO: 1013 - RNA, 22 nt, synthetic construct
  source 1..22: mol_type = other RNA
aagtacaagaacaccttcagaa

SEQ ID NO: 1014 - RNA, 22 nt, synthetic construct
  source 1..22: mol_type = other RNA
agttgaaagaattcagaatcag

SEQ ID NO: 1015 - RNA, 22 nt, synthetic construct
  source 1..22: mol_type = other RNA
tagttgaaagaattcagaatca

SEQ ID NO: 1016 - RNA, 20 nt, synthetic construct
  source 1..20: mol_type = other RNA
ctgttgcagtaatctatgag

SEQ ID NO: 1017 - RNA, 20 nt, synthetic construct
  source 1..20: mol_type = other RNA
tgcagtaatctatgagtttc

SEQ ID NO: 1018 - RNA, 18 nt, synthetic construct
  source 1..18: mol_type = other RNA
gagtcttctaggagcctt

SEQ ID NO: 1019 - RNA, 23 nt, synthetic construct
  source 1..23: mol_type = other RNA
tgccattgtttcatcagctcttt

SEQ ID NO: 1020 - RNA, 20 nt, synthetic construct
  source 1..20: mol_type = other RNA
tcctgtaggacattggcagt

SEQ ID NO: 1021 - RNA, 20 nt, synthetic construct
  source 1..20: mol_type = other RNA
cttggagtcttctaggagcc

SEQ ID NO: 1022 - RNA, 17 nt, synthetic construct
  source 1..17: mol_type = other RNA
taggtgcctgccggctt

SEQ ID NO: 1023 - RNA, 18 nt, synthetic construct
  source 1..18: mol_type = other RNA
ttcagctgtagccacacc

SEQ ID NO: 1024 - RNA, 21 nt, synthetic construct
  source 1..21: mol_type = other RNA
ctgaactgctggaaagtcgcc

SEQ ID NO: 1025 - RNA, 30 nt, synthetic construct
  source 1..30: mol_type = other RNA
ctggcttccaaatgggacctgaaaaagaac

SEQ ID NO: 1026 - RNA, 21 nt, synthetic construct
  source 1..21: mol_type = other RNA
caatttttcccactcagtatt

SEQ ID NO: 1027 - RNA, 19 nt, synthetic construct
  source 1..19: mol_type = other RNA
ttgaagttcctggagtctt

SEQ ID NO: 1028 - RNA, 22 nt, synthetic construct
  source 1..22: mol_type = other RNA
tcctcaggaggcagctctaaat

SEQ ID NO: 1029 - RNA, 16 nt, synthetic construct
  source 1..16: mol_type = other RNA
tggctctctcccaggg

SEQ ID NO: 1030 - RNA, 27 nt, synthetic construct
  source 1..27: mol_type = other RNA
gagatggctctctcccagggaccctgg

SEQ ID NO: 1031 - RNA, 17 nt, synthetic construct
  source 1..17: mol_type = other RNA
gggcactttgtttggcg

SEQ ID NO: 1032 - protein, 4 aa, synthetic construct
GGFG

SEQ ID NO: 1033 - protein, 4 aa, synthetic construct
GFGG

SEQ ID NO: 1034 - protein, 4 aa, synthetic construct
GGGF

SEQ ID NO: 1035 - protein, 4 aa, synthetic construct
FGGG

SEQ ID NO: 1036 - protein, 4 aa, synthetic construct
GGGG

SEQ ID NO: 1037 - protein, 4 aa, synthetic construct
LLQG

SEQ ID NO: 1038 - protein, 5 aa, synthetic construct
KVEPK

SEQ ID NO: 1039 - protein, 116 aa, synthetic construct
  VARIANT 13: note = X can be any amino acid
  VARIANT 42: note = X can be any amino acid
  VARIANT 84: note = X can be any amino acid
  VARIANT 88: note = X can be any amino acid
  VARIANT 93: note = X can be any amino acid
  VARIANT 111..112: note = X can be any amino acid
  VARIANT 116: note = X can be any amino acid
EVQLVESGGGLVXPGGSRKLSCAASGFTFSNYGMHWVRQAPXKGLEWVAYISSGSSTIYYADTVKGRFTISRDNAKNTLFLQMXSLRXEDTAXYYCARRGLLLDYWGOGTXXTVSX

SEQ ID NO: 1040 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSA

SEQ ID NO: 1041 - protein, 111 aa, synthetic construct
  VARIANT 15: note = X can be any amino acid
  VARIANT 87: note = X can be any amino acid
  VARIANT 89: note = X can be any amino acid
  VARIANT 108: note = X can be any amino acid
DIVLTQSPASLAVSXGQRATISCRASKSVSTSSYSYMHWYQQKPGQPPKLLIKYASYLESGVPARFSGSGSGTDFTLNIHPVEEEDXAXYYCQHSREFPWTFGGGTKXEIK

(19) United States
(12) Patent Application Publication
(10) Pub. No.: US 2026/0241023 A1
(43) Pub. Date: Aug. 20, 2026

(54) ANTIBODY OLIGONUCLEOTIDE CONJUGATES

(71) Applicant: YALE UNIVERSITY, New Haven, CT (US)

(72) Inventors: Dale L. LUDWIG, New Haven, CT (US); Poornima Bala Sahithi PAMARTHY, New Haven, CT (US); Rebaz A. AHMED, New Haven, CT (US); Venugopalareddy BOMMIREDDY VENKATA, New Haven, CT (US); Elias QUIJANO, New Haven, CT (US); Peter Glazer, New Haven, CT (US)

(73) Assignee: YALE UNIVERSITY, New Haven, CT (US)

(21) Appl. No.: 19/150,521

(22) PCT Filed: Jan. 23, 2024
(86) PCT No.: PCT/US2024/012633

§ 371 Date: Jul. 23, 2025

Related U.S. Application Data
(60) Provisional application No. 63/481,132, filed on Jan. 23, 2023.
(60) Provisional application No. 63/487,850, filed on Mar. 1, 2023.
(60) Provisional application No. 63/580,971, filed on Sep. 6, 2023.
(60) Provisional application No. 63/585,831, filed on Sep. 27, 2023.

(51) Int. Cl. A61K 47/68; A61P 35/00

(52) U.S. Cl. CPC A61K 47/6807; A61K 47/6835; A61K 47/6889; A61P 35/00

(57) ABSTRACT

The present disclosure provides antibody-oligonucleotide conjugates (AOC) having an antibody, antigen-binding fragment thereof, or antigen-binding fragment thereof, conjugated through a linker to an oligonucleotide moiety (e.g., an siRNA molecule or an antisense ASO molecule). Compositions including the AOCs and methods of using the AOCs are also provided.

### CROSS REFERENCE TO RELATED APPLICATIONS

This application claim priority to U.S. Provisional Patent Application No. 63/481,132, filed Jan. 23, 2023, U.S. Provisional Patent Application No. 63/487,850, filed Mar. 1, 2023, U.S. Provisional Patent Application No. 63/580,971, filed Sep. 6, 2023, and U.S. Provisional Patent Application No. 63/585,831, filed Sep. 27, 2023, the contents of which are hereby incorporated by reference herein, in their entireties, for all purposes.

### STATEMENT REGARDING FEDERALLY-SPONSORED RESEARCH AND DEVELOPMENT

This invention was made with government support under NIH/CA197574, awarded by National Institutes of Health. The Government has certain rights in the invention.

### SEQUENCE LISTING

The instant application contains a Sequence Listing which has been submitted herewith and is hereby incorporated by reference in its entirety. Said .xml copy, created on Jan. 23, 2024 is named 127689-5014-WO-SEQ.XML.SUPP, and is 969,195 bytes in size.

### TECHNICAL FIELD

The present disclosure relates to compositions and methods for treating cancer with antibody-oligonucleotide conjugates (AOCs).

### BACKGROUND

Antibody-oligonucleotide conjugates (AOCs) have evolved to be an important therapeutic modality in the treatment of various cancers due to their preferential delivery of chemotherapeutics to cancer cells. Although selectivity in an AOC is achieved through the antibody, non-specific toxicity is still observed. If more precise payloads could be used, the synergistic combination could potentially result in more selective and safer AOCs.

Oligonucleotides are a class of precise payloads, for example, because their sequence can be used to affect a particular genomic and/or transcriptomic location through hybridization. In this fashion, for example, oligonucleotides can be designed to inhibit protein transcription and/or translation by targeting specific genes and/or transcripts in a sequence-dependent manner. Even though oligonucleotides, e.g., siRNA and antisense oligonucleotides (ASOs), have great promise as targeted therapeutics, several challenges with their implementation exist, such as short serum stability, low membrane permeability, and lack of tissue selectivity. Antibodies, with their longer half-life, ability to selectively deliver therapeutics inside the cells, and targeting properties, are ideal partners for the targeted delivery of oligonucleotides, particularly in the form of an antibody-oligonucleotide conjugate, with a cleavable or non-cleavable linker.

The 3E10 antibody is an ideal molecular delivery vehicle due to its efficiency in penetrating into living cells with specific nuclear localization, absence of toxicity, and successful delivery of therapeutic cargo proteins in vitro and in vivo. 3E10 selectively penetrates cells expressing the nucleoside transporter ENT2. To date, in vitro and in vivo studies on 3E10 have not shown any cellular toxicity in normal cells.

There is a need to develop antibody-oligonucleotide conjugates (AOCs), which combine the high precision of siRNA and ASOs, with the deliverability of the 3E10 antibody, taking advantage of both technologies.

### SUMMARY

Given the background above, there is a need in the art for improved methods of treating cancers with therapeutic polynucleotides. Polynucleotide-based cancer therapies present a promising path for cancer therapy because of their versatility to encode any polypeptide, the availability of highly reproducible manufacturing methods, the ability to make simple and precise adjustments to polynucleotide sequences, their inexpensive nature, their ability to specifically target and/or edit any genetic sequence, etc. However, the delivery of polynucleotide therapeutics to specific tissues in vivo has posed many challenges, including the rapid degradation of foreign nucleic acids in the body and immunogenicity caused by common delivery vehicles, such as liposomes and viral vectors. See, for example, Zhou et al., Medicine in Drug Discovery, 6 (2020) 100023 and Dahlman et al., Nature Nanotechnol. 9 (8): 648-655 (2014), the contents of which are incorporated herein by reference. Advantageously, the present disclosure provides compositions, conjugates, and methods for polynucleotide-based cancer therapy that are not reliant upon liposomal or viral vector based nucleic acid delivery.

In some aspects, the disclosure provides a conjugate of Formula (I):

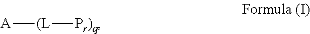

wherein in Formula (I):
A is an antibody or antigen-binding fragment thereof comprising a heavy chain variable region (VH) CDR1 comprising the amino acid sequence of SEQ ID NO:58, CDR2 comprising the amino acid sequence of SEQ ID NO:59, CDR3 comprising SEQ ID NO:60; and a light chain variable region (VL) CDR1 comprising the amino acid sequence of SEQ ID NO:61, CDR2 comprising the amino acid sequence of SEQ ID NO:62, CDR3 comprising the amino acid sequence of SEQ ID NO:63;
L is a linker;
P is an oligonucleotide moiety;
r is an integer from 1 to 4; and
q is an integer from 1 to 16.

In some embodiments, the linker L comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}—, —N═CR^{a}—, —CR^{a}═N—, —S—, —S(O)—, —S(O)₂—, —OP(O)OR^{a}—, —OP(O)OR^{a} O—, —P(O)OR^{a} O—, —O—, —CR^{b}₂—, —[(CR^{b}₂)₁₋₁₂O]₁₋₅₀—, —C(O)—, —C(S)—, —C(═N—OH)—, —C(NR^{a})—, —C(NH₂Cl)—, —C(O)O—, —OC(O)—, —C(O)S—, —SC(O)—, —C(O)NR^{a}—, —NR^{a}C(O)—, —C(O)NR^{a} SO₂—, —SO₂NR^{a}C(O)—, —OC(O)O—, —OC(O)S—, —SC(O)O—, —OC(O)NR^{a}, —NR^{a}C(O)O—, —SC(O)NR^{a}—, —NR^{a}C(O)S—, —S(O)ₜN(R^{a})— (where t is 1 or 2), —N(R^{a})S(O)ₜ— (where t is 1 or 2), and —X_{AA}—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{h} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
two independent R^{b} groups or an R^{a} and an R^{b} can be joined together to form an optionally substituted cycle; and
—X_{AA}— is an amino acid sequence comprising 1 to 6 amino acid moieties.

In some embodiments, each amino acid moiety of —X_{AA}— is independently selected from alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), citrulline (Cit), and homocitrulline (HoCit).

In some embodiments, the linker L comprises one or more groups selected from optionally substituted C₁-C₁₈ alkylene, —C≡C—, —CR^{a}— CR^{a}—, optionally substituted 6- to 14-membered arylene, optionally substituted C₃-C₂₀ cycloalkylene, —[CH₂O]₁₋₁₈—, [CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N—CR^{a}—, —CR^{a}—N—, —S—, —OP(O)OR^{a} O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—.

In some embodiments, the linker L comprises one or more groups selected from optionally substituted C₁-C₁₆ alkylene, —C≡C—, —CR^{a}═CR^{a}—, optionally substituted phenylene, optionally substituted C₃-C₆ cycloalkylene, —[CH₂CH₂O]₁₋₆—, —[CH₂CH₂CH₂O]₁₋₆—, optionally substituted 5- to 6-membered substituted 5- to 20-membered heterocycloalkylene, —NR^{a}—, —N═CR^{a}—, —CR^{a}═N—, —S—, —OP(O)OR^{a} O—, —O—, —CR^{b}₂—, —C(O)—, —C(O)O—, —OC(O)—, —C(O)NR^{a}—, —NR^{a}C(O)—, —OC(O)O—, —OC(O)NR^{a}—, —NR^{a}C(O)O—, and —X_{AA}—.

In some embodiments, the linker L comprises one or more groups selected from —[C(R^{b})₂]₁₋₆—, —C≡C—, —CR^{a}—CR^{a}—, —[CH₂CH₂O]₁₋₆—, —NR^{a}—, —N═CR^{a}—, —CR^{a}═N—, —S—, —OP(O)OR^{a} O—, —O—, —C(O)—, —C(O)O—, —OC(O)—, —C(O)NR^{a}—, —NR^{a}C(O)—, —OC(O)O—, —OC(O)NR^{a}—, —NR^{a}C(O)O—, —X_{AA}—,

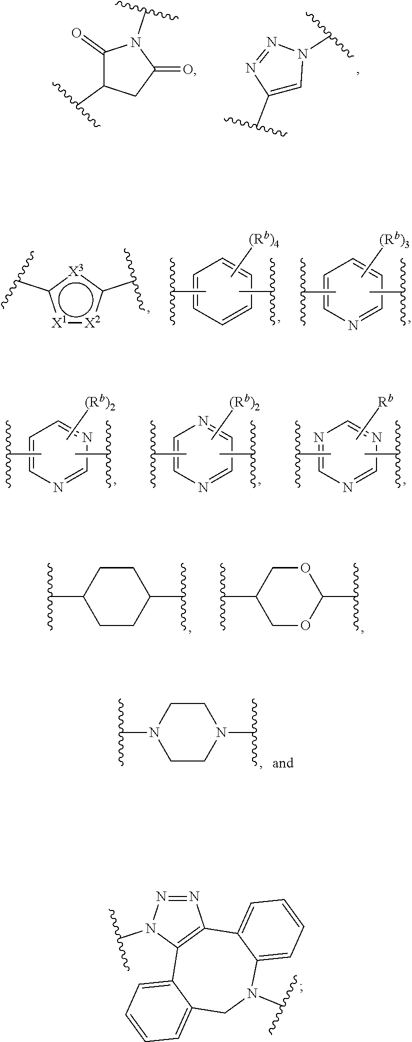

and
X¹, X², and X³ are independently selected at each occurrence from NR^{a}, N, CR^{b}, S, and O.

In some embodiments, the linker L is of Formula (L-1):

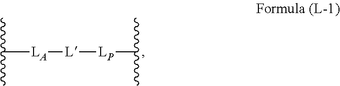

wherein in Formula (L-1):
L_{A} is a connecting moiety through which A is covalently attached to L′;
L′ is a bond or comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}—, —N═CR^{a}—, —CR^{a}═N—, —S—, —S(O)—, —S(O)₂—, —OP(O)OR^{a} O—, —O—, —CR^{b}₂—, —[(CR^{b}₂)₁₋₁₂O]₁₋₅₀—, —C(O)—, —C(S)—, —C(NR^{a})—, —C(NH₂Cl)—, —C(O)O—, —OC(O)—, —C(O)S—, —SC(O)—, —C(O)NR^{a}—, —NR^{a}C(O)—, —C(O)NR^{a} SO₂—, —SO₂NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, —S(O); N(R^{a})—, —N(R^{a})S(O)ₜ—, and —X_{AA}—; and
L_{P} is a connecting moiety through which P is covalently attached to L′.

In some embodiments, the linker L comprises at least one cleavable moiety. In some embodiments, the cleavable moiety comprises an acid-labile moiety, a reducibly-labile moiety, or an enzymatically-labile moiety. In some embodiments, the cleavable moiety comprises one or more groups selected from:

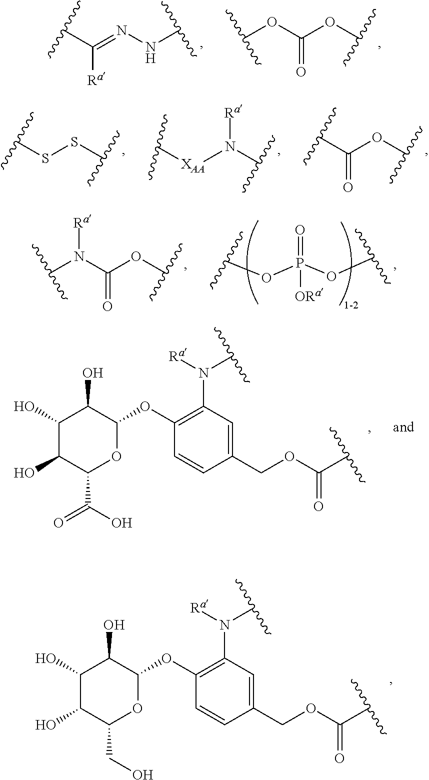

wherein:
each R^{a′} is independently selected at each occurrence from hydrogen, optionally substituted alkyl, and optionally substituted heteroalkyl.

In some embodiments, the cleavable moiety comprises the reducibly-labile moiety —S—S—.

In some embodiments, the linker L is of Formula (L-10):

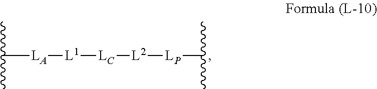

wherein in Formula (L-10):
L_{A} is selected from a bond, —NR^{a′}—, and —S—;
L′ is a bond or comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}═N—, —S—, —OP(O)OR^{a} O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—;
L_{C} is selected from an acid-labile moiety, a reducibly-labile moiety, and an enzymatically-labile moiety;
L² is a bond or comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}═N—, —S—, —OP(O)OR^{a} O—, —O—, —CR^{b}₂—, —C(O)—, —C(O)O—, —OC(O)—, —C(O)S—, —SC(O)—, —C(O)NR^{a}, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—;
L_{P} is selected from a bond, —NR^{a′}—, —S—, and —O—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{a′} is independently selected at each occurrence from hydrogen, optionally substituted alkyl, and optionally substituted heteroalkyl;
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl; and
—X_{AA}— is an amino acid sequence comprising 1 to 4 amino acid moieties.

In some embodiments, L_{C} is selected from:

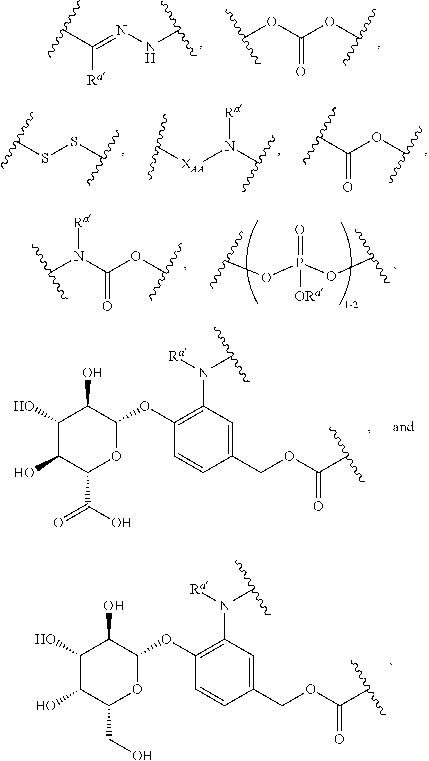

wherein:
each R^{a′} is independently selected at each occurrence from hydrogen, optionally substituted alkyl, and optionally substituted heteroalkyl.

In some embodiments, L_{C} is —S—S—.

In some embodiments, the linker L is of Formula (L-11):

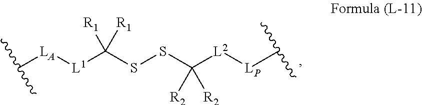

wherein in Formula (L-11):
L_{A} is selected from a bond, —NH—, and —S—;
L¹ is a bond or comprises one or more groups selected from —[C(R^{b})₂]₁₋₆—, —C≡C—, —CR^{a}—CR^{a}—, —[CH₂CH₂O]₁₋₁₆—, —NR^{a}—, —N═CR^{a}—, —CR^{a}═N—, —S—, —OP(O)OR^{a} O—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—,

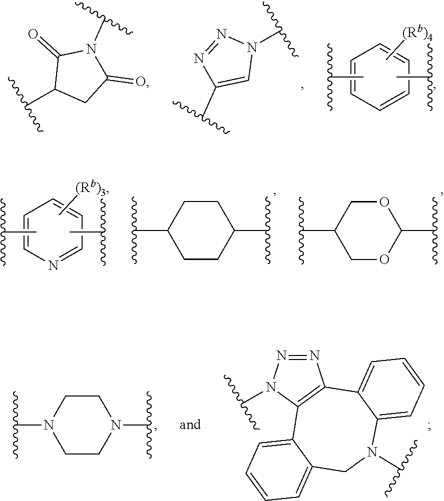

L² is a bond or comprises one or more groups selected from —[C(R^{b})₂]₁₋₆—, —C≡C—, —CR^{a}═CR^{a}—, —[CH₂CH₂O]₁₋₆—, —NR^{a}—, —N═CR^{a}—, —CR^{a}═N—, —S—, —OP(O)OR^{a} O—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—,

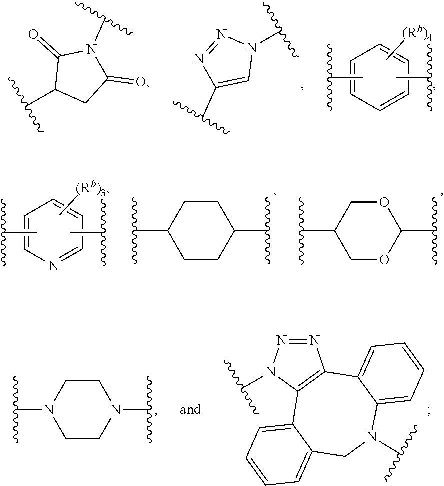

L_{P} is selected from a bond, —NR^{a′}—, and —O—;
each R₁ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₁ groups are taken together to form optionally substituted cycloalkyl;
each R₂ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₂ groups are taken together to form optionally substituted cycloalkyl;
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted alkyl;
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl; and —X_{AA}— is an amino acid sequence comprising 2 to 4 amino acid moieties.

In some embodiments, the linker L is of Formula (L-12):

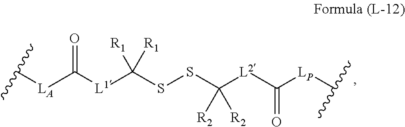

wherein in Formula (L-12):
L_{A} is selected from a bond and —NH—;
L^{1′} comprises one or more groups selected from —[C(R^{b})₂]₁₋₁₀—, —[CH₂CH₂O]₁₋₁₀—, —NR^{a}—, —O—, —C(O)—, —C(O)O—, —OC(O)—, —C(O)NR^{a}—, —NR^{a}C(O)—, —OC(O)O—, —X_{AA}—, —OC(O)NR^{a}, —NR^{a}C(O)O—,

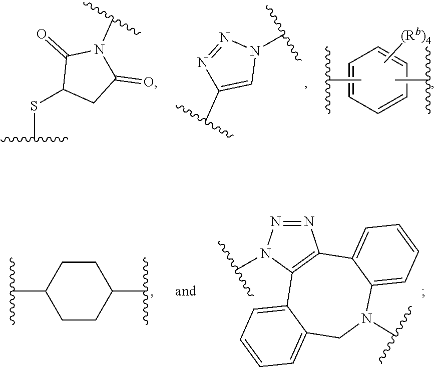

L^{2′} comprises one or more groups selected from —[C(R^{b})₂]₁₋₁₀—, —[CH₂CH₂O]₁₋₁₀—, —NR^{a}, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—,

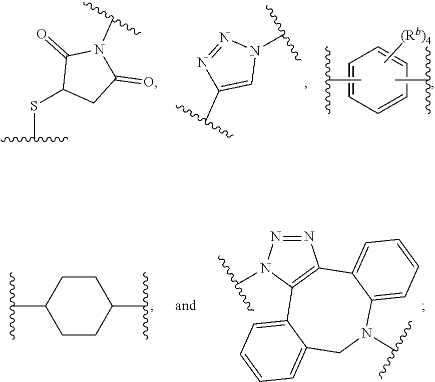

L_{P} is selected from a bond and —NR^{a′}—;
each R₁ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₁ groups are taken together to form optionally substituted C₃-C₆ cycloalkyl;
each R² is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₂ groups are taken together to form optionally substituted C₃-C₆ cycloalkyl;
each R^{a} is independently selected at each occurrence from hydrogen, optionally substituted C₁-C₆ alkyl, optionally substituted C₁-C₆ fluoroalkyl, and optionally substituted C₃-C₆ cycloalkyl;
each R^{a′} is independently selected at each occurrence from hydrogen and optionally substituted C₁-C₆ alkyl;
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₆ alkyl, optionally substituted C₁-C₆ fluoroalkyl, and optionally substituted C₃-C₆ cycloalkyl; or two independent R^{b} groups attached to the same carbon atom are taken together to form optionally substituted C₃-C₆ cycloalkyl; and —X_{AA}— is an amino acid sequence comprising 2 or 3 amino acid moieties.

In some embodiments, at least one R₁ or R₂ is other than hydrogen. In some embodiments, at least one R₁ is an optionally substituted C₁-C₈ alkyl. In some embodiments, each R₁ is independently an optionally substituted C₁-C₈ alkyl. In some embodiments, at least one R₂ is an optionally substituted C₁-C₈alkyl. In some embodiments, each R₂ is independently an optionally substituted C₁-C₈ alkyl.

In some embodiments, the linker L is selected from:

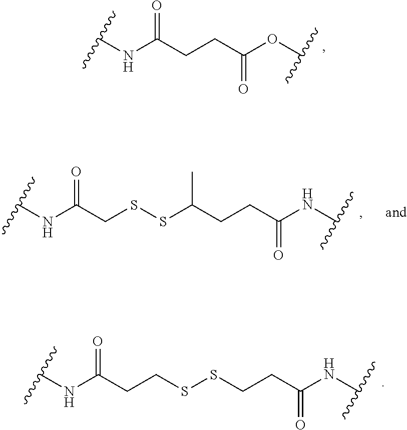 [SMILES: *NC(=O)CCC(=O)O* ; *NC(=O)CCC(C)SSCC(=O)N* ; *NC(=O)CCSSCCC(=O)N*]

In some embodiments, the linker L is selected from:

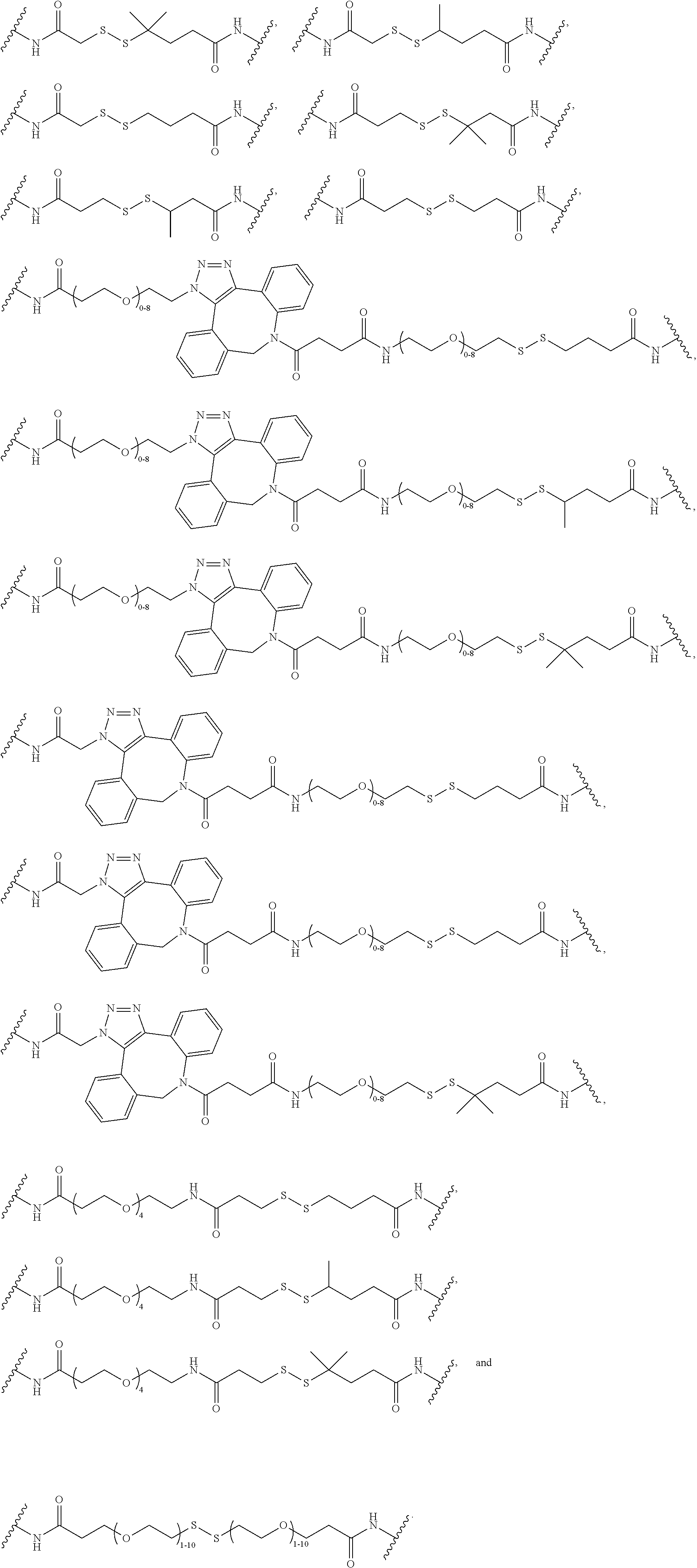

In some embodiments, the linker is a cleavable linker.

In some embodiments, the linker L is of Formula (L-20):

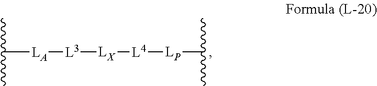

wherein in Formula (L-20):
L_{A} is selected from a bond, —NR^{a′}—, and —S—;
L³ is a bond or comprises one or more groups selected from —[C(R^{b})₂]₁₋₈—, —NR^{a}—, —C(O)—, —C(S)—, —C(NR^{a})—, —C(NH₂Cl)—, —C≡C—, —CR^{a}—CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
L_{X} comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —S—, —O—, —CR^{b}₂—, —C(O)—, —C(S)—, —C(NR^{a})—, —C(NH₂Cl)—, —C(O)O—, —OC(O)—, —C(O)S—, —SC(O)—, —C(O)NR^{a}—, and —NR^{a}C(O)—;
L⁴ is a bond or comprises one or more groups selected from —[C(R^{b})₂]₁₋₈—, —NR^{a}—, —C(O)—, —C(S)—, —C(NR^{a})—, —C(NH₂Cl)—, —C≡C—, —CR^{a}—CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
L_{P} is selected from a bond, —NR^{a′}—, —S—, and —O—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{a′} is independently selected at each occurrence from hydrogen, optionally substituted alkyl, and optionally substituted heteroalkyl; and
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl.

In some embodiments, the linker L is of Formula (L-21):

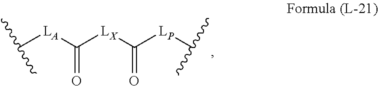

wherein in Formula (L-21):
L_{A} is selected from a bond and —NH—;
L_{X} comprises one or more groups selected from optionally substituted —[C(R^{b})₂]₁₋₆—, —C≡C—, —CR^{a}═CR^{a}—, —[CH₂CH₂CH₂O]₁₋₆—, —NR^{a}—, —O—, —C(O)—, —C(O)O—, —OC(O)—, —C(O)S—, —SC(O)—, —C(O)NR^{a}—, —NR^{a}C(O)—,

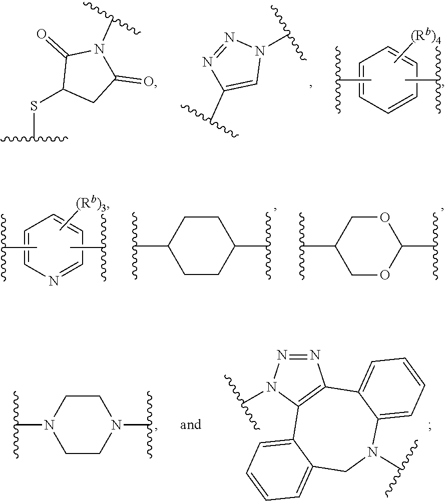

L_{P} is selected from a bond and —NR^{a′}—;
each R^{a} is independently selected at each occurrence from hydrogen, optionally substituted C₁-C₈alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

In some embodiments, the linker L is of Formula (L-22a) or Formula (L-22b):

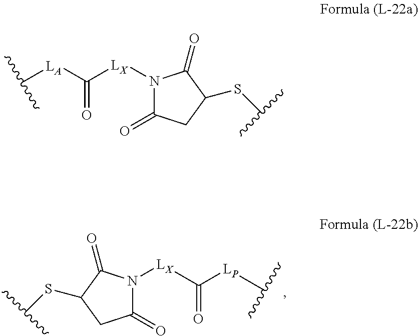

wherein in Formulas (L-22a) and (L-22b):
L_{A} is selected from a bond and —NH—;
L_{X} comprises one or more groups selected from optionally substituted —[C(R^{b})₂]₁₋₁₀—, —C≡C—, —CR^{a}—CR^{a}—, —[CH₂CH₂CH₂O]₁₋₁₀—, —NR^{a}—, —C(O)—, —C(O)O—, —OC(O)—, —C(O)S—, —SC(O)—, —C(O)NR^{a}—, —NR^{a}C(O)—,

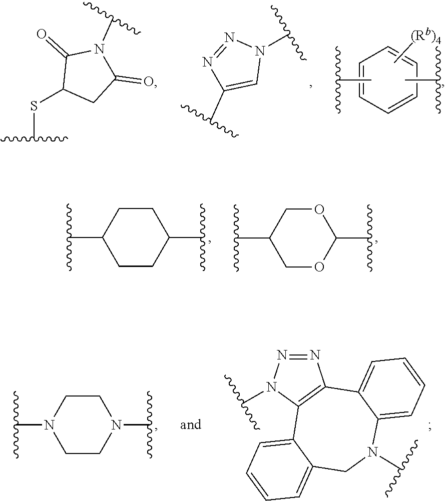

L_{P} is selected from a bond and —NR^{a′}—;
each R^{a} is independently selected at each occurrence from hydrogen, optionally substituted C₁-C₈alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

In some embodiments, the linker L is selected from:

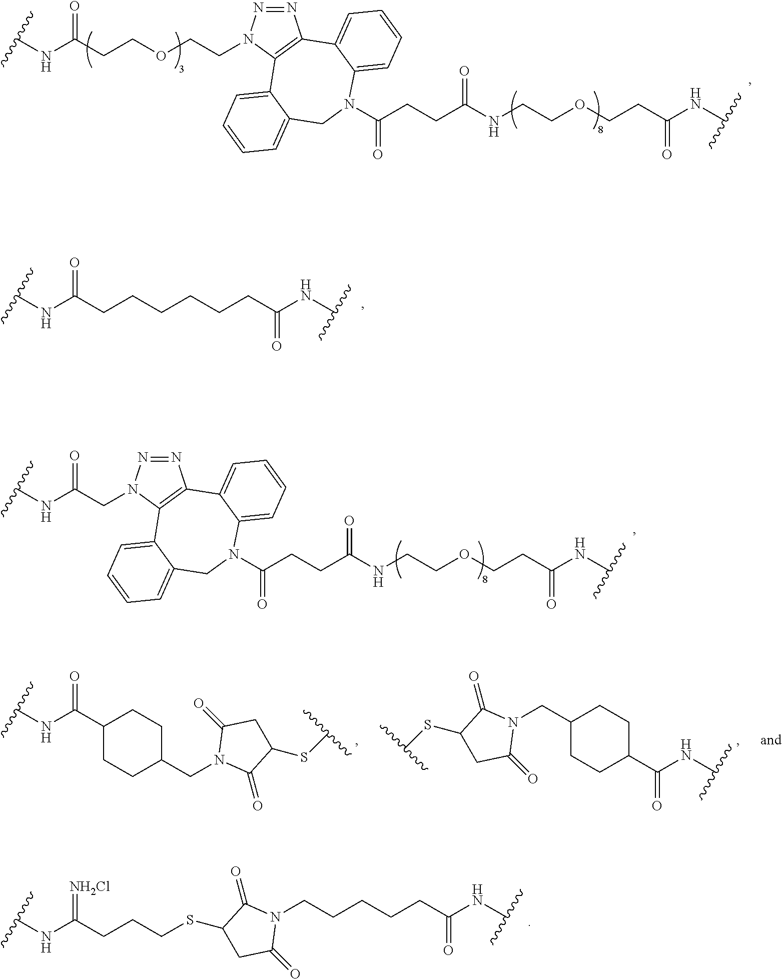

In some embodiments, the linker is a non-cleavable linker.

In some embodiments, the linker L comprises a moiety of Formula (L-100) or Formula (L-101):

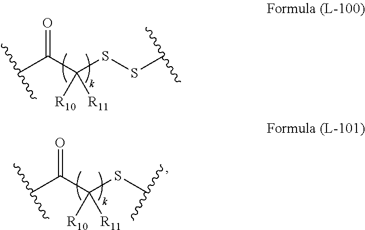

wherein in Formulas (L-100) and (L-101):
each R₁₀ is independently selected at each occurrence from hydrogen, —OH, and C₁-C₆ alkyl;
each R¹¹ is independently selected at each occurrence from hydrogen, —OH, and C₁-C₆ alkyl; and
k is 1, 2, 3, 4, 5, 6, 7, 8, 9 or 10.

In some embodiments, each R₁₀ is hydrogen; each R¹¹ is independently selected at each occurrence from hydrogen, —CH₃, —CH₂CH₃, and —CH(CH₃)₂; and k is 3, 4, 5, 6, 7, 8, 9, or 10. In some embodiments, each R₁₀ is hydrogen; each R¹¹ is independently selected at each occurrence from hydrogen, —CH₃, and —CH₂CH₃; and k is 3, 4, 5, 6, 7, 8, 9, or 10. In some embodiments, each R₁₀ is hydrogen; each R₁ is independently selected at each occurrence from hydrogen and —CH₃; and k is 3, 4, 5, 6, 7, 8, 9, or 10. In some embodiments, each R₁₀ is hydrogen; each R¹¹ is hydrogen; and k is 3, 4, 5, 6, 7, 8, 9, or 10.

In some embodiments, the linker L is selected from:

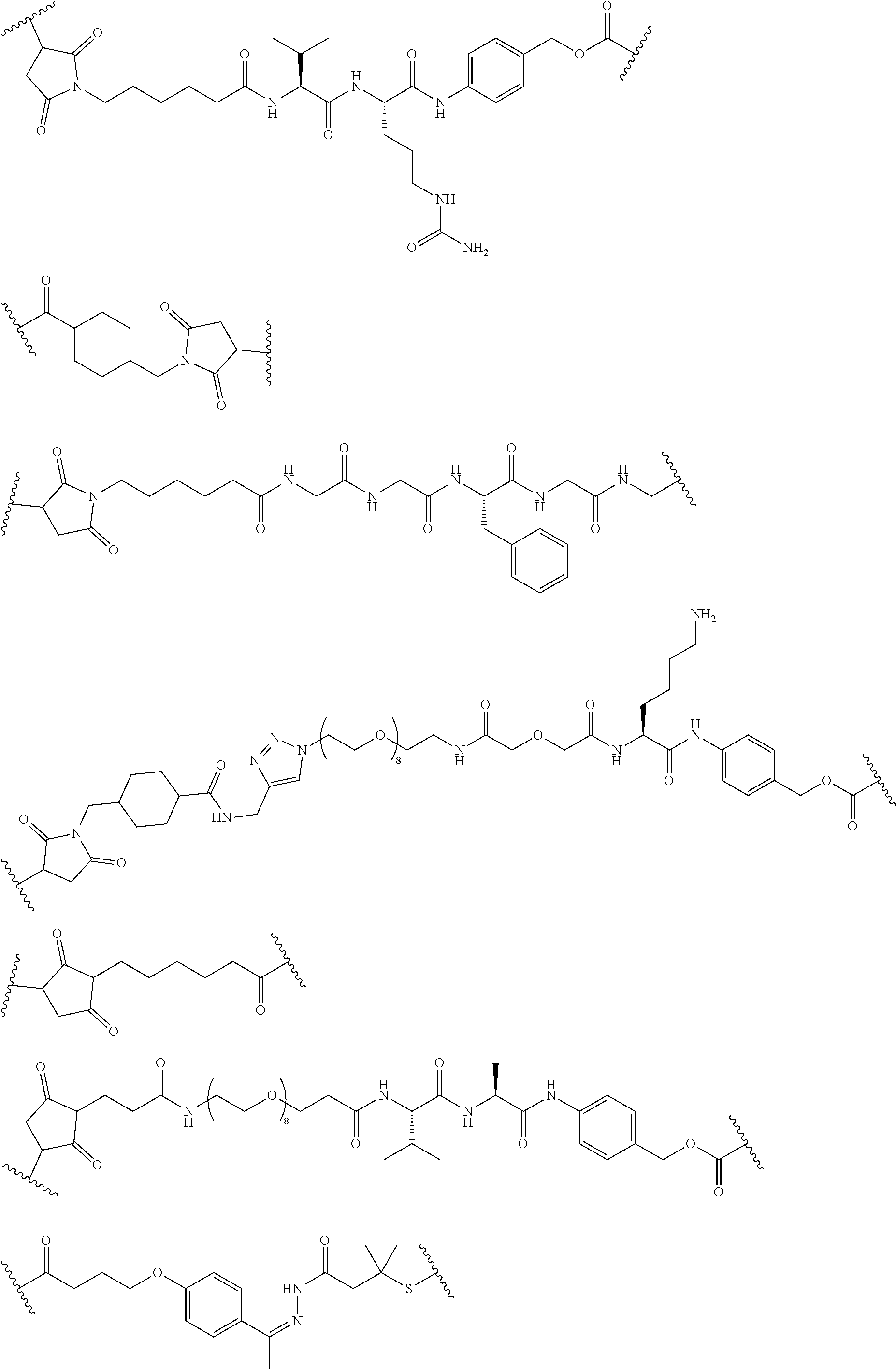

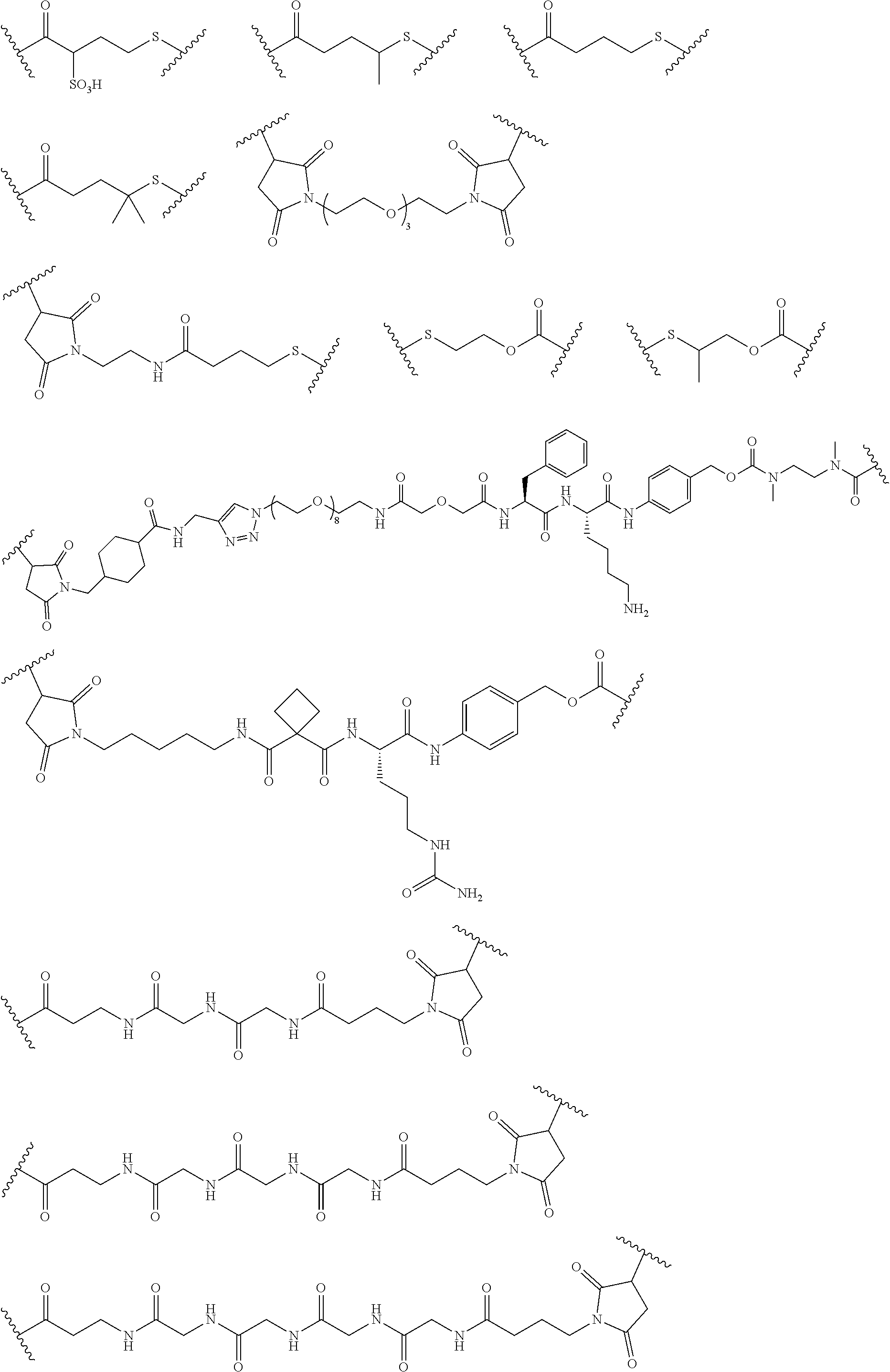

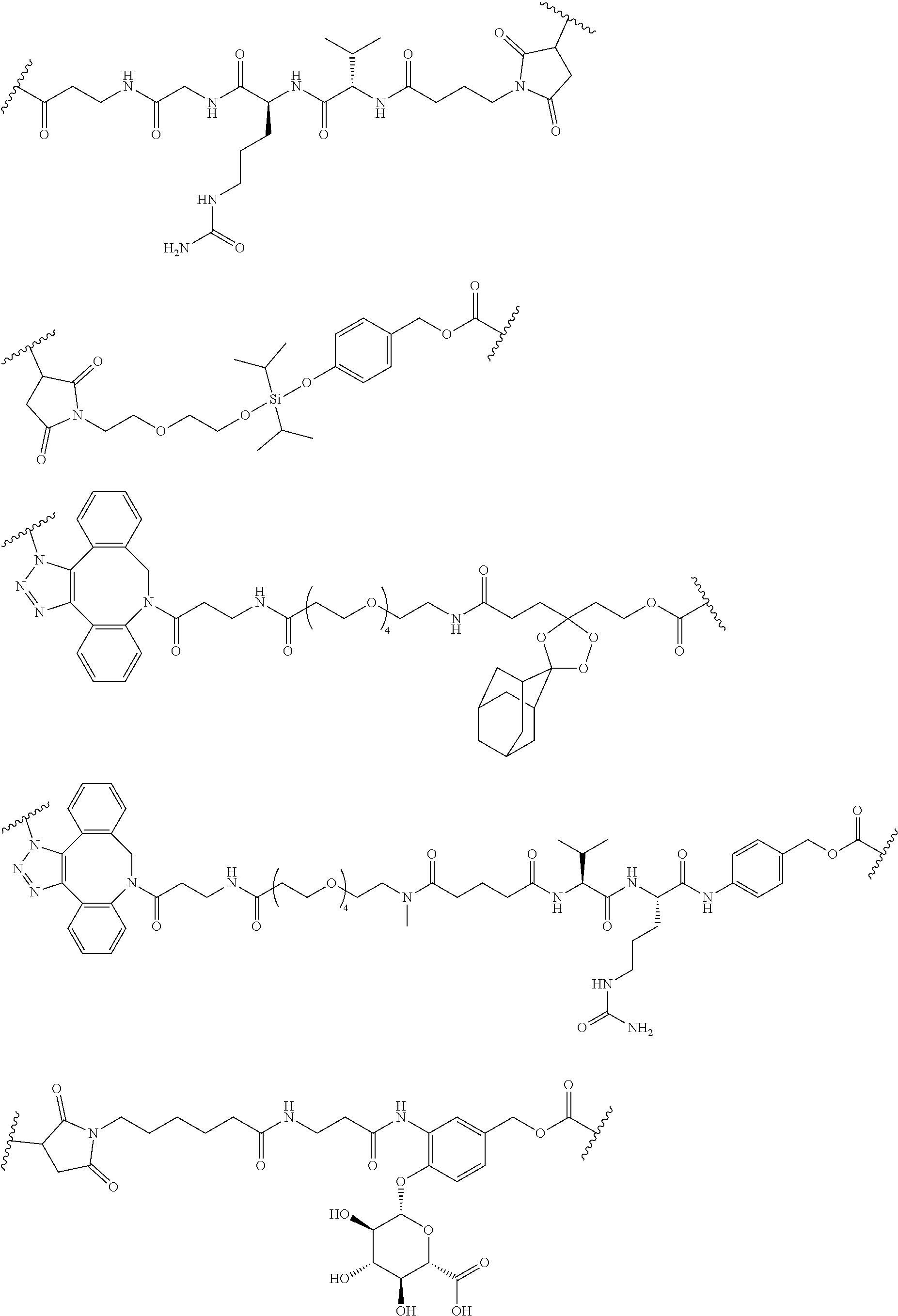

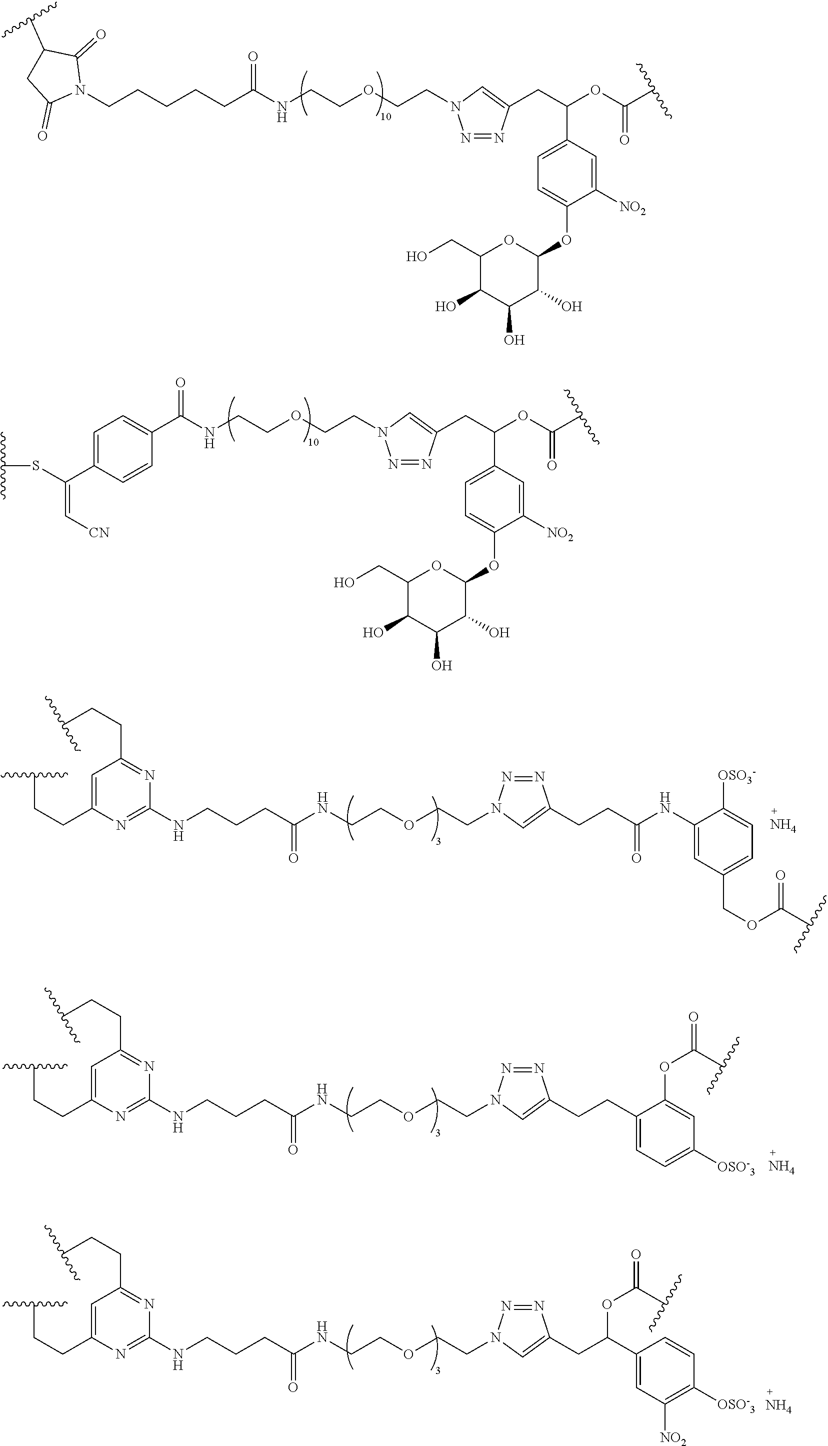

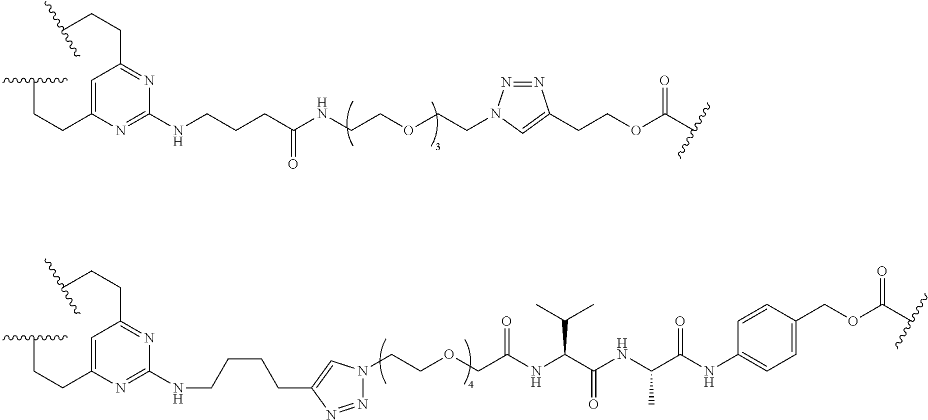

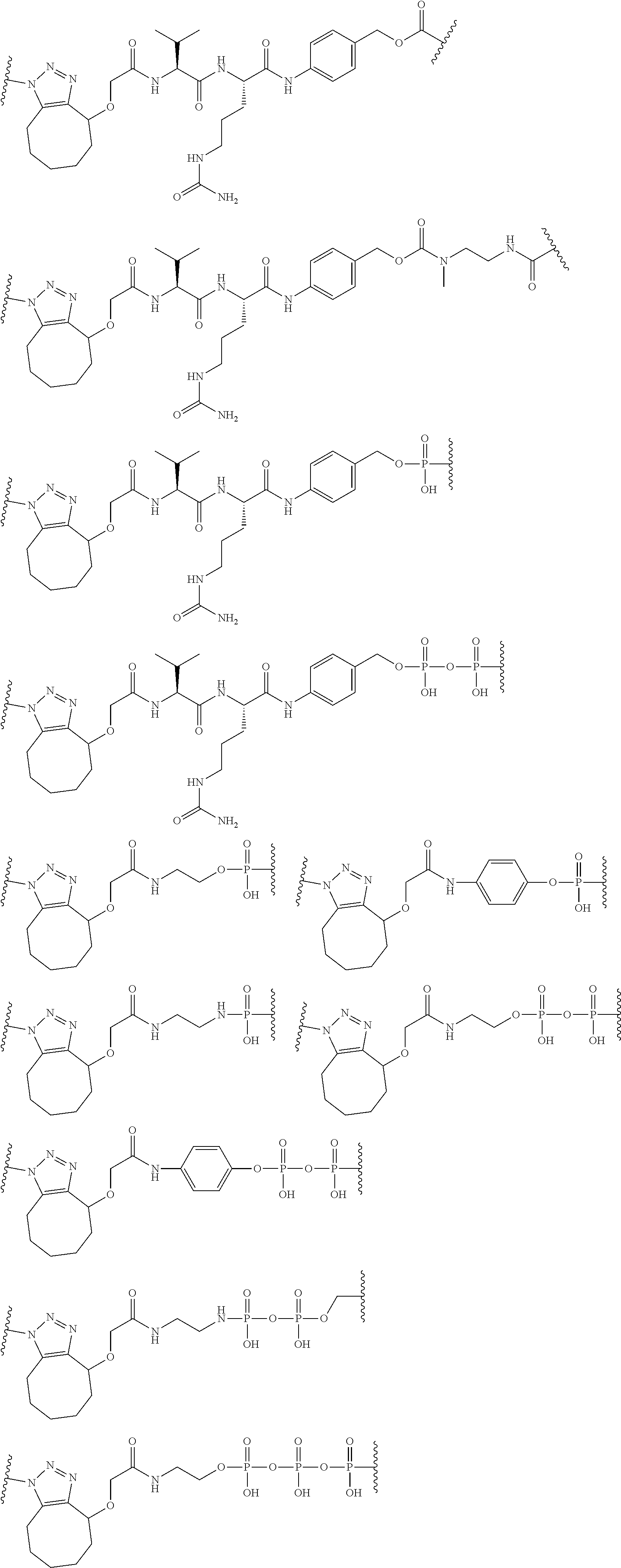

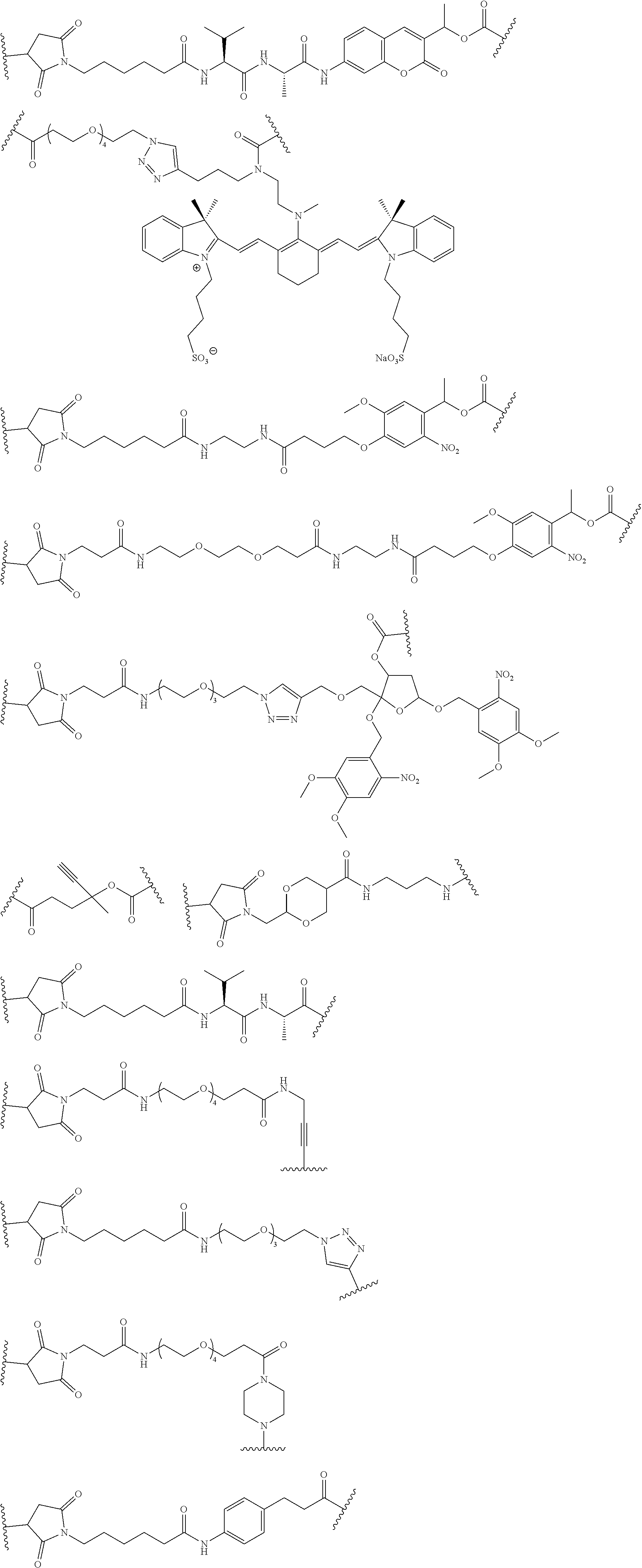

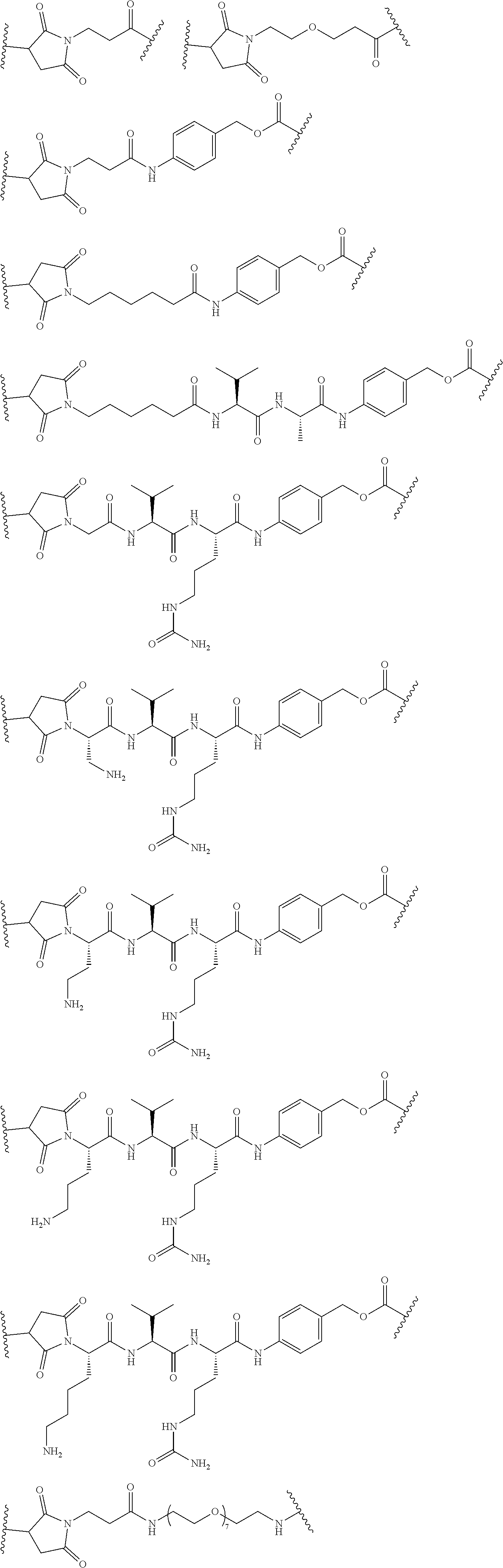

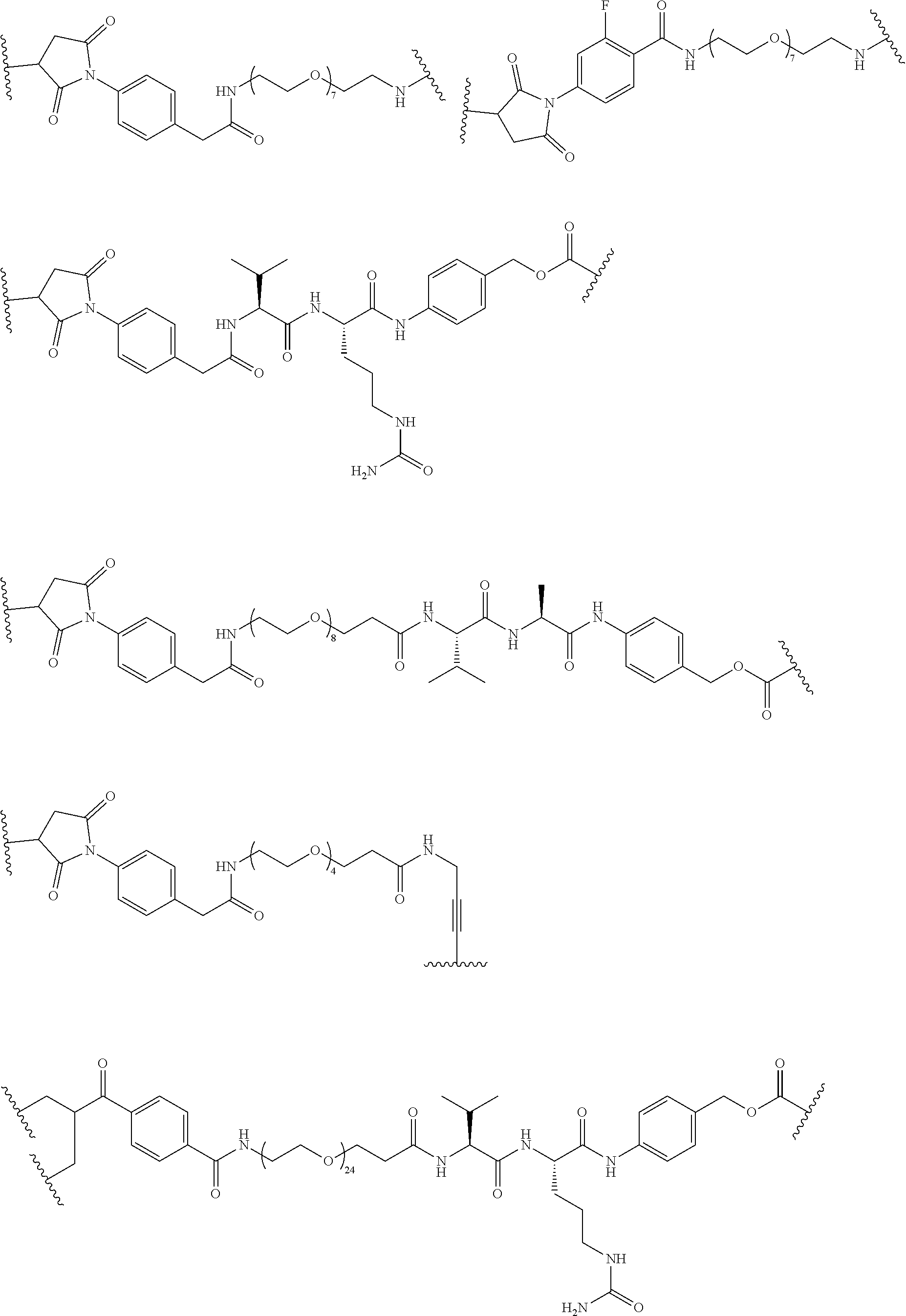

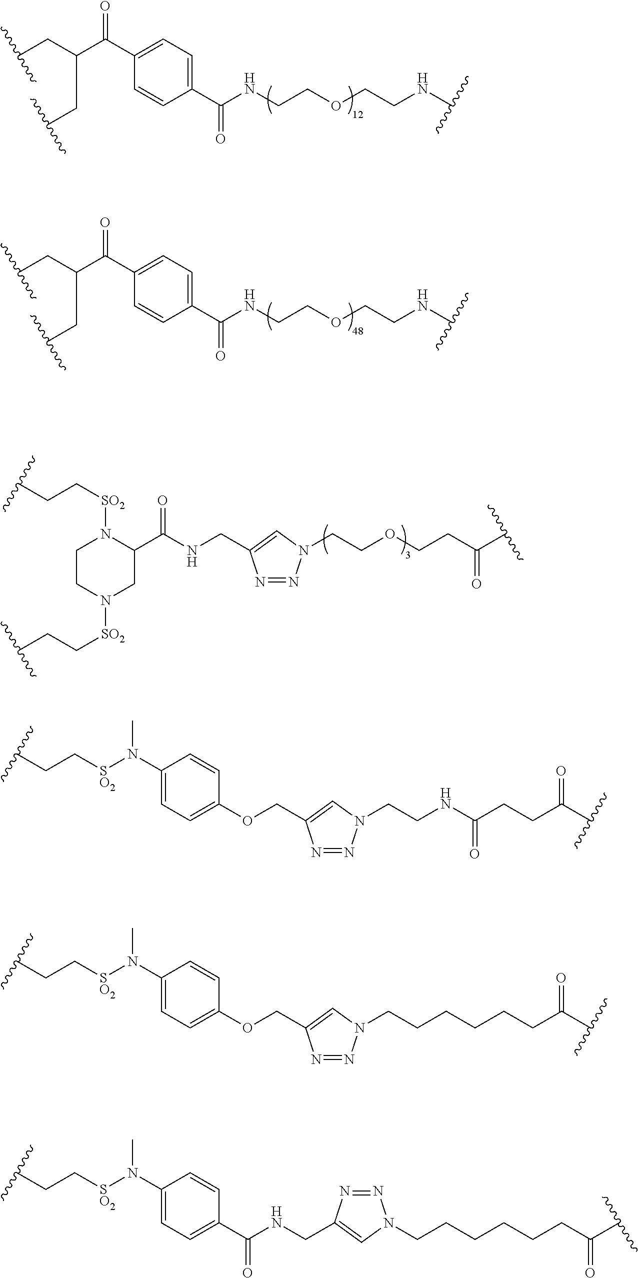

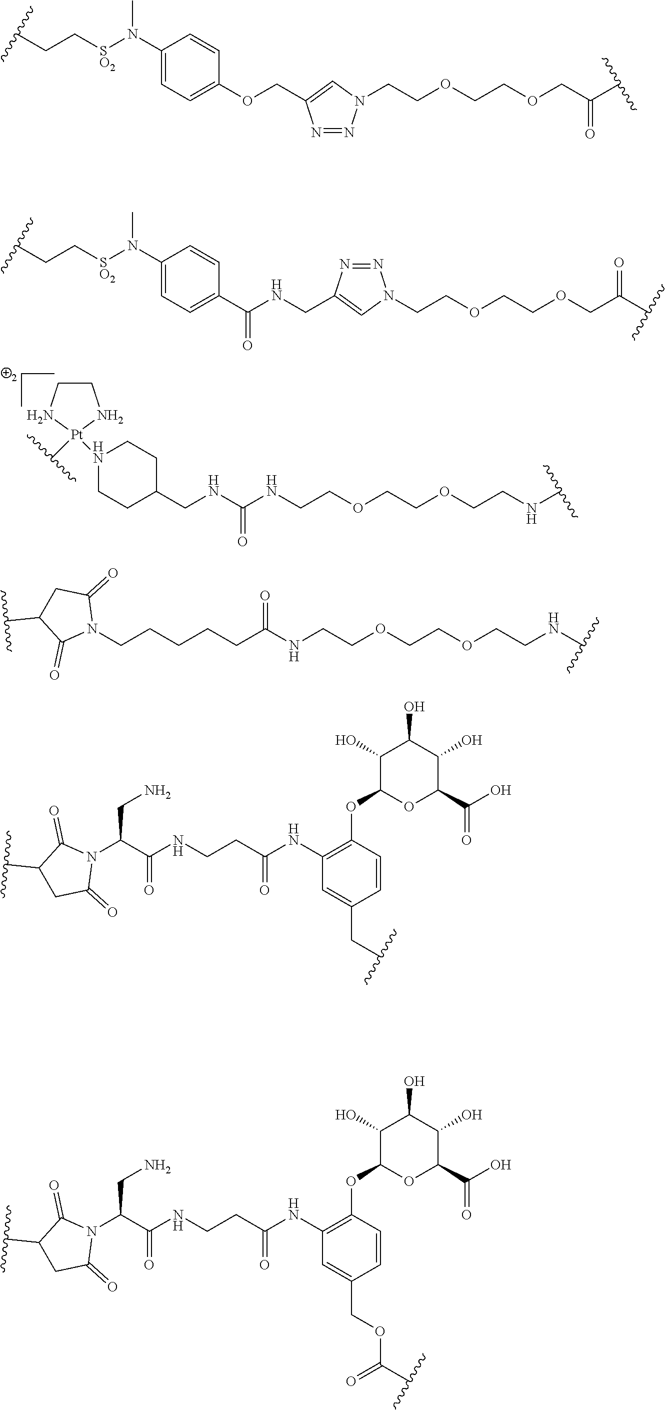

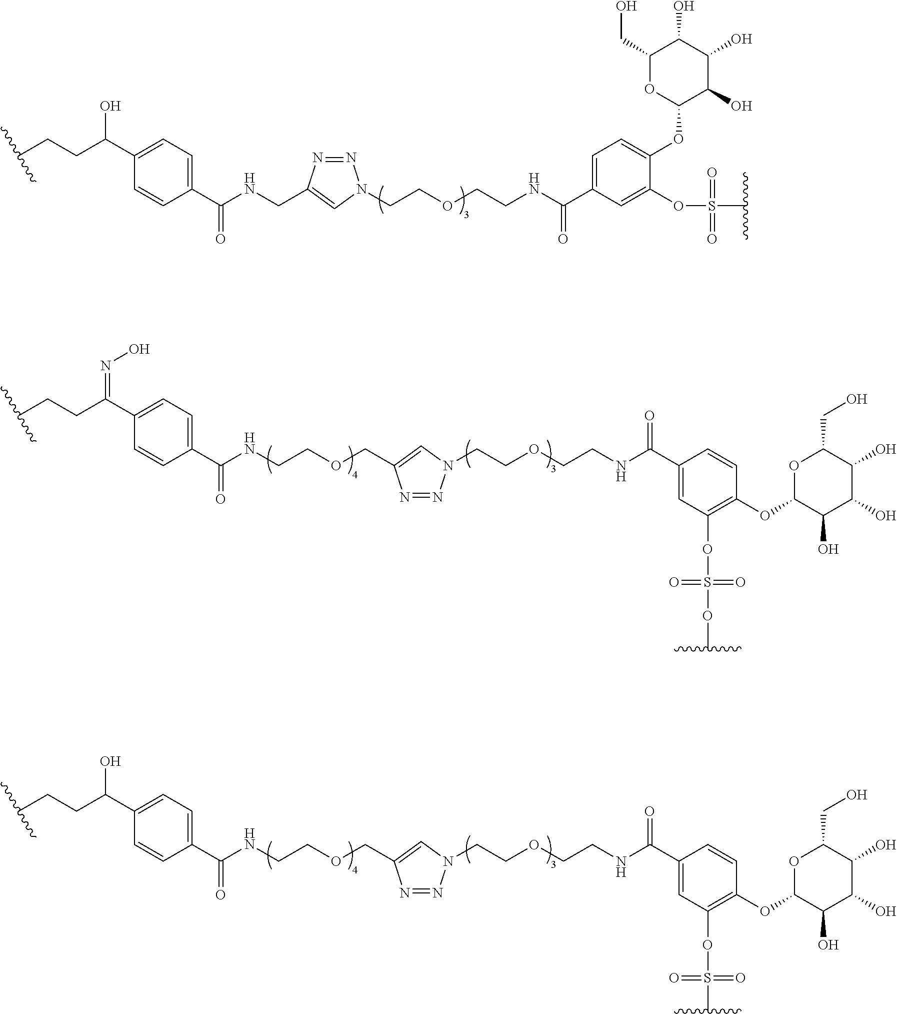

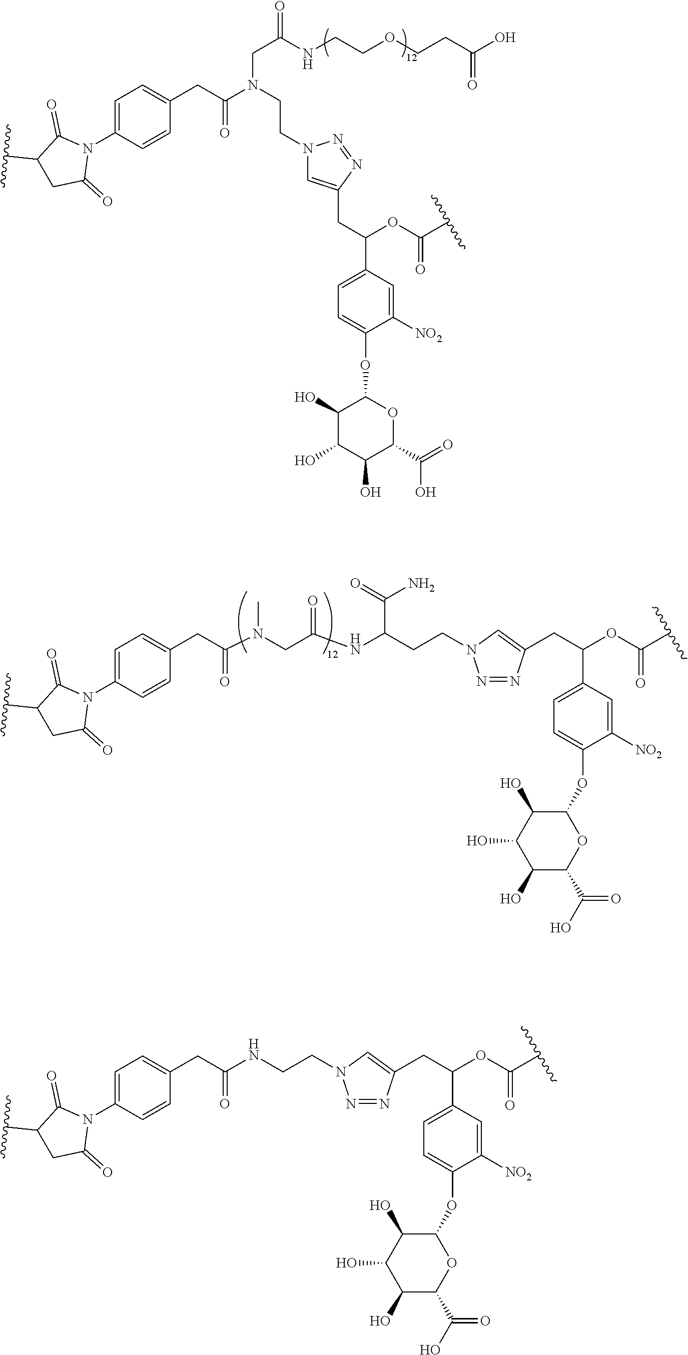

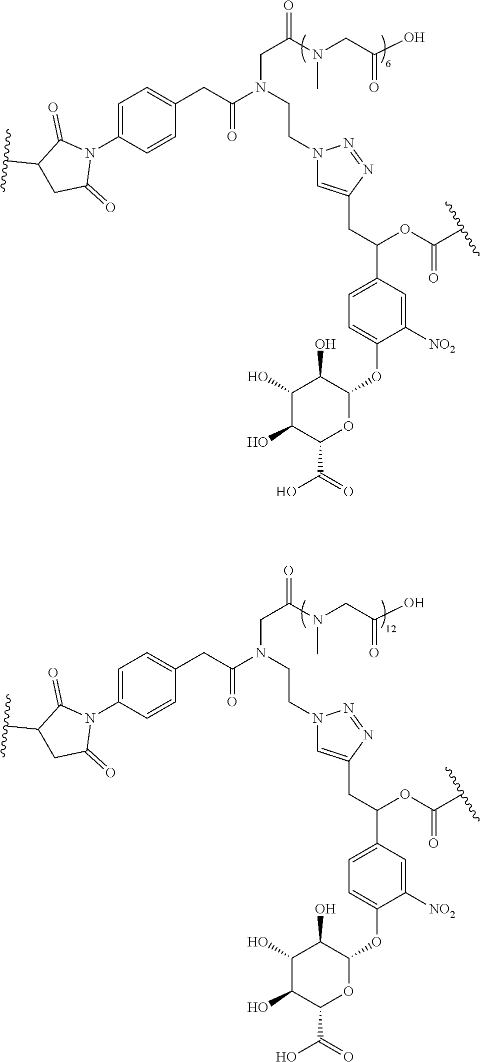

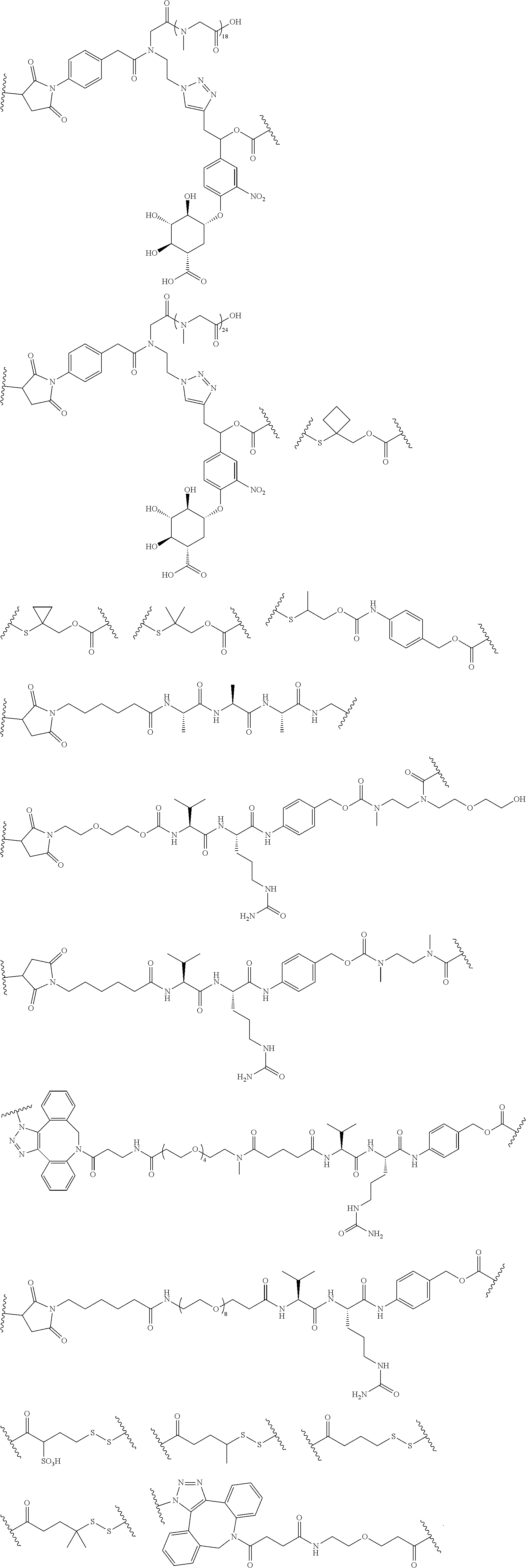

In some embodiments, each amino acid moiety of —X_{AA}— is independently selected from alanine (Ala), arginine (Arg), glycine (Gly), histidine (His), isoleucine (Ile), leucine (Leu), lysine (Lys), phenylalanine (Phe), tryptophan (Trp), tyrosine (Tyr), valine (Val), citrulline (Cit), and homocitrulline (HoCit). In some embodiments, each amino acid moiety of —X_{AA}— is independently selected from alanine (Ala), glycine (Gly), lysine (Lys), phenylalanine (Phe), valine (Val), and citrulline (Cit). In some embodiments, the amino acid sequence —X_{AA}— is selected from -Val-Cit-, —Cit-Val-, -Val-Ala-, -Ala-Val-, -Phe-Lys-, -Lys-Phe-, -Ala-Ala-, -Val-Val-, -Gly-Gly-, -Ala-Ala-Ala-, -Gly-Gly-Gly-, -Gly-Gly-Phe-Gly-(SEQ ID NO:1032), -Gly-Phe-Gly-Gly-(SEQ ID NO:1033), -Gly-Gly-Gly-Phe-(SEQ ID NO:1034), -Phe-Gly-Gly-Gly-(SEQ ID NO:1035), and -Gly-Gly-Gly-Gly-(SEQ ID NO:1036). In some embodiments, the amino acid sequence —X_{AA}— is selected from -Val-Cit-, —Cit-Val-, -Val-Ala-, -Ala-Val-, -Phe-Lys-, -Lys-Phe-, -Ala-Ala-, -Val-Val-, -Gly-Gly-, -Ala-Ala-Ala-, and -Gly-Gly-Gly-.

In some embodiments, the oligonucleotide moiety P is selected from a small interfering RNA (siRNA) molecule and an antisense oligonucleotide (ASO) molecule. In some embodiments, the oligonucleotide moiety P is a small interfering RNA (siRNA) molecule. In some embodiments, the oligonucleotide moiety P is an antisense oligonucleotide (ASO) molecule. In some embodiments, the siRNA molecule targets cMyc mRNA.

In some embodiments, the siRNA molecule comprises a nucleotide sequence selected from the group consisting of 5′-GCUUUUUUGCCCUGCGUGACCAGAT-3′ (SEQ ID NO: 138), 5′-GCCACAGCAUACAUCCUGUUU-3′ (SEQ ID NO:139), and 5′-GCCACAGCAUACAUCCUGUUU-3′ (SEQ ID NO:140), 3′-CUCGAAAAAACGGGACGCACUGGUCUA-5′ (SEQ ID NO:141), 3′-UUCGGUGUCGUAUGUAGGACA-5′ (SEQ ID NO:142), and 3′-UUCGGUGUCGUAUGUAGGACA-5′ (SEQ ID NO:143).

In some embodiments, the siRNA molecule comprises at least one 2′ modified nucleotide comprising a 2′-O-methyl, 2′-O-methoxyethyl(2′-O-MOE), 2′-O-aminopropyl, 2′-deoxy, 2′-deoxy-2′-fluoro, 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), or 2′—O—N-methylacetamido (2′-O-NMA) modified nucleotide, at least one modified internucleotide linkage, or at least one inverted abasic moiety, or a combination thereof.

In some embodiments, the siRNA molecules comprises a nucleotide sequence selected from the group consisting of: 5′-N6-G-mC-U-U-mU-U-mU-U-G-C-mC-mC-U-mG-C-G-mU-mG-A-C-C-A-G-dA-dT-3′ (SEQ ID NO:144), 5′-N6-G-C-C-A-C-A-G-C-A-U-A-C-A-U-C-C-U-G-U-U-U-3′ (SEQ ID NO:145), 5′-N6*2′FG*mC*2′FC-mA-2′FC-mA-2′FG-mC-2′FA-mU-2′FA-mC-2′FA-mU-2′FC-mC-2′FU-mG-2′FU-mU-2′FU-3′ (SEQ ID NO:146), 3′-mC-mU-mC-G-mA-A-A-A-A-A-C-G-G-G-mA-C-mG-C-A-C-U-G-G-mU-mC-mU-mA 5′ (SEQ ID NO: 147), 3′-U-U-C-G-G-U-G-U-C-G-U-A-U-G-U-A-G-G-A-C-A-5′ (SEQ ID NO: 148), and 3′-mU*2′FU*mC-2′FG-mG-2′FU-mG-2′FU-mC-2′FG-mU-2′FA-mU-2′FG-mU-2′FA-mG-2′FG-mA*2′FC*mA-5′ (SEQ ID NO:149).

In some embodiments, the siRNA molecule hybridizes to at least 8 contiguous bases of the c-Myc mRNA. In some embodiments, the siRNA molecule is from 8 to 50 nucleotides in length or from 10 to 30 nucleotides in length. In some embodiments, the siRNA molecule comprises a nucleotide sequence selected from:

| Strand | Sequence | SEQ ID NO |
|---|---|---|
| sense | GUAACCAAGAGUAUUCCAUDTDT | 152 |
| antisense | AUGGAAUACUCUUGGUUACDTDT | 153 |
| sense | UCUUGGUUACAUGAAAUCCCAUC | 154 |
| antisense | UGGGAUUUCAUGUAACCAAGA | 155 |
| sense | CAGAAAGAGUGUCUCAUCUUA | 156 |
| antisense | AAUGAUGAGACACUCUUUCUGGU | 157 |
| sense | UAUAUUUCCAGGAUGAAAGUCCA | 158 |
| antisense | GACUUUCAUCCUGGAAAUAUA | 159 |
| sense | ACAAAAGCAAAACAGGUCUAGAA | 160 |
| antisense | CUAGACCUGUTUUGCUUUUGUN | 161 |
| sense | AUGUUGUCCUUUUUAUCUGAGCAGC | 162 |
| | CGAAAGGCUGC | |
| antisense | UCAGAUAAAAAGGACAACAUGG | 163 |
| sense | GGUUAACACCAUUUACUUCAA | 164 |
| antisense | UUGAAGUAAAUGGUGUUAACCAG | 165 |
| sense | GAGAAUAUUUCACCCUUCA | 166 |
| antisense | UGAAGGGUGAAAUAUUCUC | 167 |
| sense | AGGAGUUCCACAUUCUGGC | 168 |
| antisense | GCCAGAAUGUGGAACUCCU | 169 |
| sense | UGAAGUAGAAGAUGCGCUU | 170 |
| antisense | AAGCGCAUCUUCUACUUCA | 171 |
| sense | AAACCCUUUGUCUUCUUAAAGCAGC | 172 |
| | CGAAAGGCUGC | |
| antisense | UUUAAGAAGACAAAGGGUUUGG | 173 |
| sense | CUGGAUCACAUGCACAAUGTT | 174 |
| antisense | CAUUGUGCAUGUGAUCCAGTT | 175 |
| sense | AAGCAAGAUAUUUUUAUAAUAN | 176 |
| antisense | UAUUAUAAAAAUAUCUUGCUUUUTT | 177 |

In some embodiments, the ASO molecule targets a c-Myc gene. In some embodiments, the ASO molecule targets exon 2 of the c-Myc gene. In some embodiments, the ASO molecule comprises a sequence of UUCACCATCTCUC (SEQ ID NO:150). In some embodiments, the ASO molecule comprises a sequence of mU*mU*mC*A*C*C*A*T*C*T*C*mU*mC (SEQ ID NO:151), wherein m denotes 2′-O-methyl-modified ribose nucleotides and * denotes phosphorothioate linkage.

In some embodiments, the VL CDR1 comprises the amino acid sequence of SEQ ID NO: 9, CDR2 comprises the amino acid sequence of SEQ ID NO:10, CDR3 comprises SEQ ID NO: 11; and the VH CDR1 comprises the amino acid sequence of SEQ ID NO:15, CDR2 comprises the amino acid sequence of SEQ ID NO:4, CDR3 comprises the amino acid sequence of SEQ ID NO: 5.

In some embodiments, the VL CDR1 comprises the amino acid sequence of SEQ ID NO: 29, CDR2 comprises the amino acid sequence of SEQ ID NO: 10, CDR3 comprises SEQ ID NO: 11; and the VH CDR1 comprises the amino acid sequence of SEQ ID NO:15, CDR2 comprises the amino acid sequence of SEQ ID NO:26, CDR3 comprises the amino acid sequence of SEQ ID NO:5.

In some embodiments, the antibody or antigen-binding fragment thereof comprises a light chain variable region (VL) comprising an amino acid sequence of SEQ ID NO:21 and a heavy chain variable region (VH) comprising an amino acid sequence of SEQ ID NO:14.

In some embodiments, the antibody or antigen-binding fragment thereof comprises a full length light chain (LC) comprising an amino acid sequence of SEQ ID NO:20 and a full length heavy chain (HC) comprising an amino acid sequence of SEQ ID NO:13.

In some embodiments, the antibody or antigen-binding fragment thereof comprises: a light chain variable domain (VL) comprising an amino acid sequence that is at least 95% identical to an amino acid sequence selected from the group consisting of 3E10-VL-H₁ (SEQ ID NO:85), 3E10-VL-H₂ (SEQ ID NO:86), 3E10-VL-H₃ (SEQ ID NO:87), 3E10-VL-H₄ (SEQ ID NO:88), 3E10-VL-H₅ (SEQ ID NO:89), and 3E10-VL-H₆ (SEQ ID NO:90); and a heavy chain variable domain (VH) comprising an amino acid sequence that is at least 95% identical to an amino acid sequence selected from the group consisting of 3E10-VH-H₁ (SEQ ID NO:64), 3E10-VH-H₂ (SEQ ID NO:65), 3E10-VH-H₃ (SEQ ID NO:66), 3E10-VH-H₄ (SEQ ID NO:67), 3E10-VH-H₅ (SEQ ID NO:68), 3E10-VH-H₆ (SEQ ID NO:69), and 3E10-VH-H₇ (SEQ ID NO:70).

In some embodiments, the antibody or antigen-binding fragment thereof comprises: a light chain variable domain (VL) comprising an amino acid sequence selected from the group consisting of 3E10-VL-H₁ (SEQ ID NO:85), 3E10-VL-H₂ (SEQ ID NO:86), 3E10-VL-H₃ (SEQ ID NO: 87), 3E10-VL-H₄ (SEQ ID NO:88), 3E10-VL-H₅ (SEQ ID NO:89), and 3E10-VL-H₆ (SEQ ID NO:90); and a heavy chain variable domain (VH) comprising an amino acid sequence selected from the group consisting of 3E10-VH-H₁ (SEQ ID NO:64), 3E10-VH-H₂ (SEQ ID NO: 65), 3E10-VH-H₃ (SEQ ID NO:66), 3E10-VH-H₄ (SEQ ID NO:67), 3E10-VH-H₅ (SEQ ID NO: 68), 3E10-VH-H₆ (SEQ ID NO:69), and 3E10-VH-H₇ (SEQ ID NO:70).

In some embodiments, the antibody or antigen-binding fragment thereof comprises a VL/VH pair selected from the group consisting of (a) VL1 (SEQ ID NO:85) and VH1 (SEQ ID NO: 64), (b) VL1 (SEQ ID NO:85) and VH2 (SEQ ID NO:65), (c) VL1 (SEQ ID NO:85) and VH3 (SEQ ID NO:66), (d) VL1 (SEQ ID NO:85) and VH4 (SEQ ID NO:67), (e) VL2 (SEQ ID NO:86) and VH1 (SEQ ID NO:64), (f) VL2 (SEQ ID NO:86) and VH2 (SEQ ID NO:65), (g) VL2 (SEQ ID NO: 86) and VH3 (SEQ ID NO:66), (h) VL2 (SEQ ID NO:86) and VH4 (SEQ ID NO:67), (i) VL3 (SEQ ID NO:87) and VH1 (SEQ ID NO:64), (j) VL3 (SEQ ID NO:87) and VH2 (SEQ ID NO: 65), (k) VL3 (SEQ ID NO:87) and VH3 (SEQ ID NO:66), (1) VL3 (SEQ ID NO:87) and VH4 (SEQ ID NO:67), (m) VL4 (SEQ ID NO:88) and VH1 (SEQ ID NO:64), (n) VL4 (SEQ ID NO:88) and VH2 (SEQ ID NO:65), (0) VL4 (SEQ ID NO:88) and VH3 (SEQ ID NO:66), (p) VL4 (SEQ ID NO: 88) and VH4 (SEQ ID NO:67), (q) VL5 (SEQ ID NO:89) and VH5 (SEQ ID NO:68), (r) VL5 (SEQ ID NO:89) and VH6 (SEQ ID NO:69), (s) VL6 (SEQ ID NO:90) and VH5 (SEQ ID NO: 68), and (t) VL6 (SEQ ID NO:90) and VH6 (SEQ ID NO:69).

In some embodiments, the antibody or antigen-binding fragment thereof comprises: a light chain variable domain (VL) comprising 3E10-VL-H₆ (SEQ ID NO:90) and a heavy chain variable domain (VH) comprising 3E10-VH-H₆ (SEQ ID NO:69).

In some embodiments, the antibody or antigen-binding fragment thereof comprises: a light chain variable domain (VL) comprising the amino acid sequence (DIQMTQSPSSLSASLGDRATITCRASKTVSTSSYSYMHWYQQKPGQPPKLLIKYASYLE SGVPSRFSGSGSGTDFTLTISSLQPEDAATYYCQHSREFPWTFGGGTKVEIK) (SEQ ID NO: 117) and a heavy chain variable domain (VH) comprising the amino acid sequence (EVOLVESGGGLVQPGGSLRLSCAASGFTFSNYGMHWVRQAPGKGLEWVSYISSGSSTI YYADSVKGRFTISRDNAKNSLYLQMNSLRAEDTAVYYCARRGLLLDYWGQGTTVTVS S) (SEQ ID NO:105).

In some aspects, the present disclosure provides a method for treating a subject in need thereof, the method comprising administering a therapeutically effective amount of a conjugate of the disclosure to the subject.

### BRIEF DESCRIPTION OF THE DRAWINGS

FIG. 1 (SEQ ID NOs: 1-12) illustrates amino acid sequences for the parent 3E10 monoclonal antibody.

FIGS. 2A (SEQ ID NOs: 13-25), 2B (SEQ ID NOs: 26-30), and 2C (SEQ ID NOs: 31-33) illustrate amino acid sequences for the D31N variant (FIG. 2A), other CDR variants (FIG. 2B), and additionally contemplated CDR variants (FIG. 2C) of the 3E10 monoclonal antibody, in accordance with some embodiments of the present disclosure.

FIG. 3 (SEQ ID NOs: 34-57) illustrates example charge-conserved CDR variants of the 3E10 monoclonal antibody, in accordance with various embodiments of the present disclosure.

FIG. 4 (SEQ ID NOs: 58-63) illustrates example CDR variants containing a combination of amino acid substitutions, charged-conserved amino acid substitutions, and rationally-designed amino acid substitutions of the 3E10 monoclonal antibody, in accordance with various embodiments of the present disclosure.

FIG. 5 (SEQ ID NOs: 64-70) illustrates amino acid sequences of humanized 3E10 variable heavy (3E10-VH) domains, in accordance with various embodiments of the present disclosure.

FIG. 6 (SEQ ID NOs: 71-77) illustrates amino acid sequences of mature humanized 3E10 heavy chains (3E10-HC), lacking a signal peptide, in accordance with various embodiments of the present disclosure.

FIG. 7 (SEQ ID NOs: 78-84) illustrates amino acid sequences of humanized 3E10 heavy chains (3E10-HC), in accordance with various embodiments of the present disclosure.

FIG. 8 (SEQ ID NOs: 85-90) illustrates amino acid sequences of humanized 3E10 variable light (3E10-VL) domains, in accordance with various embodiments of the present disclosure.

FIG. 9 (SEQ ID NOs: 91-96) illustrates amino acid sequences of mature humanized 3E10 light chains (3E10-LC), lacking a signal peptide, in accordance with various embodiments of the present disclosure.

FIG. 10 (SEQ ID NOs: 97-102) illustrates amino acid sequences of humanized 3E10 light chains (3E10-LC), in accordance with various embodiments of the present disclosure.

FIG. 11 shows the schematic representations of the evaluated antibody-oligonucleotide conjugates (AOCs) 3E10-SATA-SPP-cMyc, 3E10-Az/DBCO-PEG8-cMyc, 3E10-DSP-cMyc, and 3E10-C6-cMyc.

FIG. 12 shows the results of Myc luciferase activity in HCT1116 cells treated with the antibody-oligonucleotide conjugates 3E10-SATA-SPP-cMyc, 3E10-Az/DBCO-PEG8-cMyc, 3E10-DSP-cMyc, and 3E10-C6-cMyc. The humanized antibody (V66) alone, or PBS served as controls and luciferase activity is normalized to PBS control.

FIG. 13 shows the results of Myc luciferase activity in HCT1116 cells treated with a 3E10-cMyc siRNA conjugate with a cleavable linker (i.e., 3E10-SATA-SPP-cMyc). The humanized antibody (V66) alone, or PBS served as controls and luciferase activity is normalized to PBS control.

FIG. 14 shows micrographs of HCT1116 cells treated with PBS (Vehicle), 2.5 μM 3E10 (V66), 0.625 μM 3E10 (V66)-cMyc siRNA conjugate, 1.25 μM 3E10 (V66)-cMyc siRNA conjugate, 2.5 μM 3E10 (V66)-cMyc siRNA conjugate.

FIG. 15 shows dose response curves measuring in vitro Myc luciferase activity in a HCT116 cell line treated with 3E10-Myc-siRNA.

FIG. 16 shows the results of a Cell Titer Glo assay in OVCAr8 cells treated with a humanized 3E10 antibody (V66), a 3E10-cMyc siRNA conjugate with a cleavable linker.

FIG. 17 shows micrographs of OVCAr8 cells treated with PBS (Vehicle), 2.5 UM 3E10 (V66), 0.625 μM 3E10 (V66)-cMyc siRNA conjugate, 1.25 μM 3E10 (V66)-cMyc siRNA conjugate, 2.5 μM 3E10 (V66)-cMyc siRNA conjugate.

FIG. 18 (SEQ ID NOs: 103-112) illustrates a sequence alignment of examples of humanized 3E10 heavy chain variable regions, with CDRs underlined as indicated.

FIG. 19 (SEQ ID NOs: 113-121) illustrates a sequence alignment of examples of humanized 3E10 light chain variable regions, with CDRs and putative nuclear localization signals (NLS) underlined as indicated.

FIGS. 20A, 20B, 20C, 20D, and 20E (SEQ ID NOs: 122-137) collectively illustrate a sequence alignment of example of humanized di-scFv constructs of the 3E10 monoclonal antibody.

FIGS. 21A, 21B, 21C, and 21D collectively show the effect of 3E10-D31N antibody (V66) therapy as a single agent, in a non-covalent complex with a c-Myc antisense oligonucleotide (ASO), and in combination with the chemotherapeutic, carboplatin, on a xenograft OVCAR-8 ovarian tumor model.

FIG. 22A (SEQ ID NO:1038) shows the Lys-azide conjugation intermediates of 3E10-D31N monoclonal antibody (V66) after proteolysis.

FIG. 22B shows the mass spectroscopy results of mapped Lys-azide conjugation intermediates of 3E10-D31N monoclonal antibody (V66) after proteolysis.

FIG. 22C (SEQ ID NOs: 1039, 1040, 69, 1041, 8 and 90) shows the sequence of humanized 3E10-D31N monoclonal antibody (V66) and 3E10-D31N.

FIGS. 23A and 23B collectively illustrate improved cellular internalization of 3E10-D31N monoclonal antibody (V66) oligonucleotide conjugates in A427 cells utilizing trasglutaminase-mediated enzymatic conjugation.

FIG. 24A shows improved exon skipping utilizing transglutaminase-mediated enzymatic conjugation. FIG. 24B shows varying the length of the PEG linkers (e.g., PEG4, PEG8, and PEG12) did not significantly impact exon skipping. FIG. 24C shows enhanced exon-skipping of cleavable linkers, protease Cathepsin-B and SPDMV (disulfide) compared to non-cleavable linkers with transglutaminase-mediated enzymatic conjugation.

FIG. 25A show a di-methyl-hindered disulfide cleavable linker and protease cleavable linker have greater stability over a single methyl-hindered disulfide linker in mouse serum. FIG. 25B show a data table indicating the panel of antibody-oligonucleotide conjugates (AOCs) tested and their oligonucleotide to antibody ratios (OAR).

FIG. 26 shows a summary table of the in vitro performance of several antibody-oligonucleotides conjugates utilizing lysine and transglutaminase-mediated conjugation.

### DETAILED DESCRIPTION

### I. Introduction

The present disclosure provides compositions, conjugates, and methods for delivering therapeutic oligonucleotides to tissues expressing ENT2. In some embodiments, the methods and compositions find particular use for the treatment of cancers. For instance, compositions comprising a conjugate of (i) a 3E10 antibody or antigen-binding fragment thereof, and (ii) a therapeutic oligonucleotides, as well as methods for using such compositions for the treatment of cancers, are described.

The studies described herein demonstrate that AOCs comprising a cell-penetrating and nucleic acid-binding 3E10 antibody conjugated via a linker to an oligonucleotide can effectively penetrate and kill tumor cells with specificity. For example, Examples 1 and 2 demonstrate that a 3E10-cMyc siRNA conjugate with a cleavable linker was able to effectively knock down c-Myc expression in a human colon cancer cell line. Example 3 demonstrates that a 3E10-cMyc siRNA conjugate with a cleavable linker significantly inhibited cell viability of the human ovarian cancer cell line, OVCAR8, in a dose dependent manner. Example 4 demonstrates that a combination of a 3E10-cMyc siRNA conjugate with carboplatin significantly reduced tumor volumes in a xenograft OVCAR-8 ovarian tumor model.

### II. Definitions

The terminology used in the present disclosure is for the purpose of describing particular embodiments only and is not intended to be limiting.

As used herein, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.” Additionally, where the terms “comprising,” “including,” “includes,” “having,” “has,” “with,” or variants thereof are used in either the detailed description and/or the claims, alternatives reciting “consisting of” or “consisting essentially of” are intended to be encompassed within such disclosures.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

As used herein, the term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The term “antibody,” as used herein, is used in the broadest sense and encompasses monoclonal antibodies (including full length monoclonal antibodies), polyclonal antibodies, multispecific antibodies (e.g., bispecific antibodies), chimeric antibodies, humanized antibodies, human antibodies, fusion proteins comprising an antigen determination portion of an antibody, and antibody fragments (such as Fab, Fab′, F(ab′)₂, Fv fragments, scFv molecules), and any other modified immunoglobulin molecule comprising an antigen recognition site, so long as they exhibit one or more of the desired biological activities. In embodiments, “desired biological activity” of an antibody refers to the ability of the antibody to bind to its target antigen, e.g., a nucleic acid, e.g., DNA. In embodiments, “desired biological activity” can further include antibody binding to its target antigen and resulting in a measurable biological response which can be measured in vitro or in vivo. Such activity can be antagonistic or agonistic. In embodiments, “desired biological activity” of an antibody refers to the ability of the antibody to bind to a target, e.g., nucleic acid molecules. In embodiments, “desired biological activity” of an antibody refers to the ability of the antibody to bind to a cellular receptor, e.g., ENT2. In embodiments, “desired biological activity” of an antibody refers to the ability of the antibody to be internalized by a target cell. “Target antigen,” as used herein, refers to the molecule that is bound specifically by the antigen-binding domain comprising the variable regions of a given antibody. The term “specifically binds” refers to the binding of an antibody to its cognate antigen (e.g., a nucleic acid, e.g., DNA) while not significantly binding to other antigens.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these can be further divided into subclasses or isotypes, e.g., IgG₁, IgG₂, IgG₃, IgG₄, IgA₁, and IgA₂. “Isotype,” as used herein, refers to any of the subclasses of immunoglobulins defined by the chemical and antigenic characteristics of their constant regions. The heavy chain constant domains that correspond to the different classes of immunoglobulins are called α, γ, ε, γ, and μ, respectively. The subunit structures and three-dimensional configurations of different classes of immunoglobulins are well known and described generally in, for example, Abbas et al. Cellular and Mol. Immunology, 4^{th} ed. (W.B. Saunders, Co., 2000). It should be understood that antibodies disclosed herein can also comprise hybrids of isotypes and/or subclasses.

Antibodies of the present disclosure are generally isolated or recombinant. “Isolated,” when used to describe the various polypeptides disclosed herein, refers to a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. As used herein, “recombinant antibody” refers to an antibody that is generated using recombinant nucleic acid techniques in exogenous host cells, and recombinant antibodies can be isolated as well.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The term “constant domain” refers to the portion of an immunoglobulin molecule having a more conserved amino acid sequence relative to the other portion of the immunoglobulin, the variable domain, which contains the antigen-binding site. The constant domain contains the CH₁, CH₂ and CH₃ domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) or complementary determining regions (CDRs), both in the light-chain and the heavy-chain variable domains, that confer antigen specificity. A “variable heavy domain” pairs with a “variable light domain” to form an antigen-binding domain (ABD) that specifically binds a target antigen. The more highly conserved portions of variable domains are called the framework regions (FR). The variable domains of native heavy and light chains each comprise four FR regions, largely adopting a beta-sheet configuration, connected by three CDRs/HVRs, which form loops connecting, and in some cases forming part of, the beta-sheet structure. The CDRs/HVRs in each chain are held together in close proximity by the FR regions and, with the CDRs/HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The terms “hypervariable region,” “HVR,” “HV,” “complementary determining region,” and “CDR,” used interchangeably herein, refer to the regions of an antibody variable domain which are hypervariable in sequence and/or form structurally defined loops. Generally, antibodies comprise six HVRs or CDRs; three in the VH (H₁, H₂, H₃; or VH CDR1, VH CDR2, VH CDR3), and three in the VL (L1, L2, L3; or VL CDR1, VL CDR2, VL CDR3).

The “light chains” of antibodies (immunoglobulins) from any mammalian species can be assigned to one of two clearly distinct types, called kappa (“κ”) and lambda (“λ”), based on the amino acid sequences of their constant domains.

Together, the CDRs of the VH and VL domains form an Fv region. In embodiments, a VH and a VL domain comprise the six CDRs of the ABD. In a “Fab” format, the variable heavy domain (VH; containing VH CDR1, VH CDR2, and VH CDR3) and the variable light domain (VL or VL; containing the VL CDR1, VL CDR2 and VL CDR3), comprise the set of 6 CDRs, with the C-terminus of the VH domain being attached to the N-terminus of the CH₁ domain of the heavy chain and the C-terminus of the VL domain being attached to the N-terminus of the constant light domain (and thus forming the light chain). In an “scFv” format, the VH and VL domains are covalently attached, generally through the use of a linker (e.g., an “scFv linker”), into a single polypeptide sequence, which can have the N- to C-terminus arrangement of VH-linker-VL or VL-linker-VH. In general, the C-terminus of the scFv domain is attached to the N-terminus of the hinge in the second monomer.

“Fab” or “Fab region,” as used herein, refers to a polypeptide that comprises VH, CH₁, VL, and CL immunoglobulin domains, generally on two different polypeptide chains (e.g., VH-CH₁ on one chain and VL-CL on the other). Fab can refer to this region in isolation, or this region in the context of an antibody of the disclosure. In embodiments, a Fab comprises an Fv region in addition to CH1 CL domains.

Another part of the heavy chain is the hinge region. As used herein, “hinge,” “hinge region,” “antibody hinge region,” or “hinge domain” refers to the flexible polypeptide comprising the amino acids between the first and second constant domains of an antibody. Structurally, the IgG CH1 domain ends at EU position 215, and the IgG CH₂ domain begins at residue EU position 231. Thus, for IgG, the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

“Heavy chain constant region,” as used herein, refers to the CH₁-hinge-CH₂-CH₃ portion of an antibody or fragment thereof, excluding the variable heavy domain. In embodiments, the heavy chain constant region comprises amino acids 118-447 of human IgG1, in EU numbering. As used herein, “heavy chain constant region fragment” refers to a heavy chain constant region that contains fewer amino acids from either or both of the N- and C-termini but still retains the ability to form a dimer with another heavy chain constant region.

“Fv,” “Fv fragment,” or “Fv region,” as used herein, refers to a polypeptide that comprises VL and VH domains of an antibody binding domain. Fv regions can be formatted as both Fabs and scFvs, where the VL and VH domains are combined (e.g., by way of a linker, as discussed herein) to form an scFv.

“Fc,” “Fc region,” or “Fc domain,” as used herein, refers to a polypeptide comprising CH₂-CH₃ domains of an IgG molecule, and, in some cases, inclusive of the hinge. In EU numbering for human IgG1, the CH₂-CH₃ domain comprises amino acids 231 to 447, and the hinge is 216 to 230. Thus, the definition of “Fc domain” includes both amino acids 231-447 (CH₂—CH₃) and 216-447 (hinge-CH₂-CH₃) of IgG1, or fragments thereof. An “Fc fragment” in this context can contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.). In embodiments, the disclosed AOCs comprise human Fc domains. In embodiments, the disclosed AOCs comprise Fc domains from human IgG1, IgG2, or IgG4.

A “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In embodiments, variant Fc domains have at least about 80%, at least about 85%, at least about 90%, at least about 95%, at least about 97%, at least about 98%, or at least about 99% identity to the corresponding parental human IgG Fc domain. In embodiments, the percent identity is calculated using the identity algorithms discussed below. In embodiments, the percent identity is calculated using the BLAST algorithm known in the art, using default parameters. In embodiments, variant Fc domains have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. In embodiments, variant Fc domains retain the ability to form a dimer with Ir Fc domain as measured using known techniques as described herein, such as non-denaturing gel electrophoresis.

For all positions discussed in the present disclosure that relate to antibodies, unless otherwise noted, amino acid position numbering is according to the EU index. The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5^{th} edition, NIH publication, No. 91-3242, E. A. Kabat et al.; Edelman et al., 1969, Proc Natl Acad Sci USA 63:78-85, the contents of which are incorporated herein by reference. In embodiments of the present disclosure, amino acid position numbering is according to the IMGT system.

The terms “full length antibody,” “intact antibody” and “whole antibody” are used herein interchangeably to refer to an antibody in its substantially intact form, not antibody fragments as defined below. The terms particularly refer to an antibody with heavy chains that contain an Fc region.

An “antibody fragment” comprises a portion of an intact antibody, preferably comprising the antigen-binding region thereof. Examples of antibody fragments include Fab, Fab′, F(ab′)₂, and Fv fragments; diabodies; linear antibodies; single-chain antibody molecules; and multispecific antibodies formed from antibody fragments.

A “naked antibody” for the purposes herein is an antibody that is not conjugated to a payload, e.g., an oligonucleotide, cytotoxic moiety, or radiolabel.

The term “monoclonal antibody” as used herein refers to an antibody obtained from a population of substantially homogeneous antibodies, e.g., the individual antibodies comprising the population are identical except for possible mutations, e.g., naturally occurring mutations, that can be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this disclosure. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

Antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit one or more of the desired biological activities (see, e.g., U.S. Pat. No. 4,816,567; and Morrison et al., Proc. Natl. Acad. Sci. USA 81:6851-6855 (1984)). Typically, the variable region of both light and heavy chains corresponds to the variable region of antibodies derived from one species of mammals (e.g., mouse, rat, rabbit, etc.) with the desired specificity, affinity, and/or capability, while the constant regions are homologous to the sequences of antibodies derived from another species of mammals (e.g., human) to avoid eliciting an immune response In that species. Chimeric antibodies include PRIMATTZED® antibodies wherein the antigen-binding region of the antibody is derived from an antibody produced by, e.g., immunizing macaque monkeys with the antigen of interest.

“Humanized” forms of non-human (e.g., murine) antibodies are chimeric antibodies that contain minimal sequence derived from non-human immunoglobulin. In embodiments, a humanized antibody is a human immunoglobulin (recipient antibody) in which residues from a CDR/HVR of the recipient are replaced by residues from a CDR/HVR of a non-human species (donor antibody) such as mouse, rat, rabbit, or nonhuman primate having the desired specificity, affinity, and/or capacity. In some instances, FR residues of the human immunoglobulin are replaced by corresponding non-human residues. Furthermore, humanized antibodies can comprise residues that are not found in the recipient antibody or in the donor antibody. These modifications can be made to further refine antibody performance. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See, e.g., Jones et al., Nature 321:522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409. Examples of methods used to generate humanized antibodies are described in U.S. Pat. No. 5,225,539 or 5,639,641, incorporated herein by reference in their entireties.

As used herein, the term “human antibody” refers to an antibody which possesses an amino acid sequence which corresponds to that of an antibody produced by a human and/or has been made using any technique known in the art. This definition of a human antibody includes intact or full-length antibodies, fragments thereof, and/or antibodies comprising at least one human heavy and/or light chain polypeptide. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147 (1): 86-95 (1991). See also van Dijk and van de Winkel, Curr. Opin. Pharmacol., 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “species-dependent antibody” is one which has a stronger binding affinity for an antigen from a first mammalian species than it has for a homologue of that antigen from a second mammalian species. Normally, the species-dependent antibody “binds specifically” to a human antigen (e.g., has a binding affinity (Kd) value of no more than about 1×10⁻⁷M, preferably no more than about 1×10⁻⁸M and preferably no more than about 1×10⁻⁹M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above, but preferably is a humanized or human antibody.

The expression “linear antibodies” refers to the antibodies described in Zapata et al. (1995 Protein Eng, 8 (10): 1057-1062). Briefly, these antibodies comprise a pair of tandem Fd segments (VH—CH1-VH—CH1) which, together with complementary light chain polypeptides, form a pair of antigen-binding regions. Linear antibodies can be bispecific or monospecific.

“Modification,” as used herein, refers to an amino acid substitution, insertion, deletion, and/or any other mutation in a polypeptide sequence.

“Variant protein,” or “protein variant,” or “variant,” as used herein refers to a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., described herein, are generally at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% identical to the parent polypeptide, using any alignment program known in the art, such as BLAST.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters.

In embodiments, a parent polypeptide, for example an Fc parent polypeptide, is a human wild type sequence, such as the heavy constant domain or Fc region from IgG1, IgG2, IgG3 or IgG4, although human sequences with variants can also serve as “parent polypeptides.” In embodiments, antibody sequences described herein have at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 99.5% sequence identity with a parent polypeptide sequence. Accordingly, “antibody variant” or “variant antibody” as used herein refers to an antibody that differs from a parent antibody by virtue of at least one amino acid modification; “IgG variant” or “variant IgG” as used herein refers to an IgG that differs from a parent IgG (e.g., from a human IgG sequence) by virtue of at least one amino acid modification; “immunoglobulin variant” or “variant immunoglobulin” as used herein refers to an immunoglobulin sequence that differs from that of a parent immunoglobulin sequence by virtue of at least one amino acid modification; and “Fc variant” or “variant Fc” as used herein refers to an Fc that differs from a parent Fc, e.g., an Fc domain of human IgG1, IgG2, IgG3, or IgG4, by virtue of at least one amino acid modification.

“IgG subclass modification” or “isotype modification,” as used herein, refers to amino acid modifications that convert one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

“Non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

As used herein, “oligonucleotide” or “polynucleotide,” used interchangeably, refers to a linear polymer of natural or modified nucleoside monomers linked by phosphodiester bonds or analogs thereof. The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides and polynucleotides can be natural or synthetic. Oligonucleotides and polynucleotides can include deoxyribonucleosides, ribonucleosides, and/or non-natural analogs thereof. In embodiments, oligonucleotides or polynucleotides are capable of specifically binding to a target genome by way of a regular pattern of monomer-to-monomer interactions, such as Watson-Crick type of base pairing, base stacking, Hoogsteen or reverse Hoogsteen types of base pairing, or the like. As used herein, “functional nucleic acid” refers to a nucleic acid having biological functions in vivo or in cells, such as enzymatic functions, catalytic functions, or biologically inhibiting or enhancing functions (e.g., inhibition or enhancement of transcription or translation). In embodiments, examples of functional nucleic acids include, but are not limited to, siRNA, ASO, shRNA, miRNA (including pri-miRNA and pre-miRNA), nucleic acid aptamers (including RNA aptamers and DNA aptamers), ribozymes (including deoxyribozymes), riboswitches, Ul adaptors, molecular beacons, and transcriptional factor-binding regions.

As used herein, a “3E10 antibody” refers to an antibody with a set of heavy chain CDRs (VH CDR1, VH CDR2, and VH CDR3), identified according to the Kabat system, comprising amino acid sequences that vary from SEQ ID NOS: 58, 59, and 60 by no more than two amino acids each, respectively, a set of light chain CDRs (VL CDR1, VL CDR2, and VL CRD3) comprising amino acid sequences that vary from SEQ ID NOS: 61, 62, and 63 by no more than two amino acids each, respectively, that binds nucleic acids and is cell-penetrating at least when bound to a nucleic acid, as well as antigen-binding fragments thereof. As described herein, the 3E10 antigen is a polynucleotide.

As used herein, the term “cell-penetrating” refers to an antibody or antigen binding fragment thereof that can penetrate a cell, e.g., a mammalian cell, without the aid of an exogeneous transport vehicle, such as a liposome, or a conjugated cell-penetrating peptide. With respect to 3E10 antibodies and antigen binding fragments thereof, the cell-penetrating antibody or antigen binding fragment thereof can penetrate a cell expressing an ENT2 receptor on its cell surface in the presence of nucleic acids, e.g., non-covalently bound and/or conjugated to the 3E10 antibody or antigen binding fragment thereof, resulting in internalization of the 3E10 antibodies and antigen binding fragments thereof. In some embodiments, the cell-penetrating 3E10 antibody or antigen binding fragment thereof is conjugated to a functional molecule, e.g., a chemical agent, polynucleotide, or polypeptide. Although the cell-penetrating molecules are generally referred to herein as “cell-penetrating antibodies,” it will be appreciated that fragments, including antigen-binding fragments, variants, binding proteins and fusion proteins such as scFv, di-scFv, tri-scFv, and other single chain variable fragments, and other cell-penetrating molecules disclosed herein are also expressly provided for use in compositions, conjugates, and methods disclosed herein. Autoantibodies against double-stranded deoxyribonucleic acid (dsDNA) are frequently identified in the serum of patients with systemic lupus erythematosus (SLE) and are often implicated in disease pathogenesis. Therefore, in embodiments, cell-penetrating antibodies (e.g., cell-penetrating anti-DNA antibodies) can be derived orisolated from patients with SLE or animal models of SLE.

As used herein, “antibody-oligonucleotide conjugate” or “AOC” refers to an antibody or antigen-binding fragment thereof that is covalently linked or conjugated to a biologically active molecule, for example an oligonucleotide or anti-tumor oligonucleotide, for example, an siRNA molecule, an antisense oligonucleotide.

As used herein, a “linker” is any chemical moiety that is capable of linking or connecting a molecule, including an oligonucleotide, to a cell-binding agent such as an antibody, such as a 3E10 antibody or a fragment thereof, in a stable, covalent manner. In embodiments, a “linker” is any chemical moiety that is capable of linking or connecting a compound such as an oligonucleotide, a polynucleotide, a DNA damage-inducing agent, a DNA repair inhibitor, an immune modulatory molecule, an alkylating agent, a microtubule inhibitor, an immune checkpoint inhibitor, an angiogenesis inhibitor, an adoptive cell therapy, or a topoisomerase inhibitor, to a cell-binding agent such as a 3E10 antibody or a fragment thereof, in a stable, covalent manner. Linkers can be susceptible to or be substantially resistant to acid-induced cleavage, light-induced cleavage, peptidase-induced cleavage, esterase-induced cleavage, and/or disulfide bond cleavage, at conditions under which the compound and/or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and know in the art.

As used herein, the term “subject” means any individual who is the target of administration. The subject can be any animal (e.g., a mammal. Thus), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment). In embodiments, the subject is a human. In embodiments, methods of the disclosure are useful in treatment a human subject. In embodiments, the human may be referred to as a patient. In embodiments, the human is a female. In embodiments, the human is a male. In embodiments, the human has an age in a range of from about 1 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

The terms “cancer” and “cancerous” refer to or describe the physiological condition in mammals in which a population of cells are characterized by unregulated cell growth. Examples of cancer include, but are not limited to, colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumors.

“Tumor” and “neoplasm” refer to any mass of tissue that results from excessive cell growth or proliferation, either benign (noncancerous) or malignant (cancerous), including pre-cancerous lesions.

The terms “cancer cell,” “tumor cell,” and grammatical equivalents thereof refer to the total population of cells derived from a tumor or a pre-cancerous lesion, including both non-tumorigenic cells, which comprise the bulk of the tumor cell population, and tumorigenic stem cells (cancer stem cells).

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. In embodiments, the carrier or excipient is selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein “antibody-oligonucleotide conjugate” or “AOC” refers to an antibody or antigen-binding fragment thereof that is conjugated via a linker to a therapeutic oligonucleotide, for example, an oligonucleotide, an siRNA, or an antisense oligonucleotide (ASO), which may be delivered to specific cells or tissues otherwise not targetable by oligonucleotide delivery. The conjugation of an oligonucleotide with an antibody or antigen-binding fragment thereof may also improve the pharmacokinetic properties of therapeutic oligonucleotides, expanding application of this therapeutic modality.

A “pharmacologically effective amount,” “pharmacologically effective dose,” “therapeutically effective amount,” or “effective amount” refers to an amount sufficient to produce the desired physiological effect or amount capable of achieving the desired result, particularly for treating or preventing the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures, tissue samples, tissue homogenates or experimental animals, e.g., for determining the LD50 (the dose lethal to about 50% of the population) and the ED50 (the dose therapeutically effective in about 50% of the population) or the maximum tolerated dose. The dosage can vary depending upon the dosage form employed and the route of administration utilized. The dose ratio between toxic and therapeutic effects is the therapeutic index and can be expressed as the ratio LD50/ED50. In embodiments, compositions, conjugates, and methods that exhibit large therapeutic indices are preferred. A therapeutically effective dose can be estimated initially from in vitro assays, including, for example, cell culture assays or measurements. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In embodiments, the effect will result in a quantifiable change of at least about 10%, at least about 20%, at least about 30%, at least about 50%, at least about 70%, or at least about 90%. In embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

### III. Antibody-Oligonucleotide Conjugates (AOCs)

The present disclosure is directed, in part, to antibody-oligonucleotide conjugates (AOCs) comprising a cell-penetrating antibody, e.g., a 3E10 antibody or antigen-binding fragment thereof, conjugated via a linker to an oligonucleotide, e.g., a therapeutic oligonucleotide. In embodiments, an AOC described herein has the formula A-(L-Pᵣ)_{q}, wherein: A is a 3E10 antibody or antigen-binding fragment thereof, L is a linker, and P is an oligonucleotide moiety as described herein.

In embodiments, the present disclosure relates to the use of 3E10 antibodies, and derivatives thereof, for delivering therapeutic agents to a subject. Although generally referred to herein as “3E10” or “3E10 antibodies,” it will be appreciated that fragments, variants, and binding proteins, including antigen-binding fragments and fusion proteins, such as scFv, di-scFv, tr-scFv, and other single chain variable fragments, and other cell-penetrating, nucleic acid transporting molecules disclosed herein, are encompassed by the phrase and are also expressly provided for use in compositions, conjugates, and methods disclosed herein. Thus, the antibodies and other binding proteins are also referred to herein as cell-penetrating.

In some aspects, the antibody is conjugated to the biologically active molecule via a linker. In some aspects, the antibody is a 3E10 antibody or antigen-binding fragment thereof, as described herein. In some aspects, the antibody is a humanized 3E10 antibody or antigen-binding fragment thereof, as described herein. Any variety of agents can be transported via conjugation to the 3E10 antibody or antigen-binding fragment thereof, or humanized 3E10 antibody or antigen-binding fragment thereof, herein, such as inorganic and organic molecules, pharmaceutical agents, drugs, peptides, proteins, genetic material, and the like. In some aspects, the antibody-oligonucleotide conjugate (AOC) comprises an oligonucleotide.

### A. Antigen-Binding Domains (ABDs)

As used herein, the term “antigen-binding domain” or “ABD” refers to a domain comprising a three-dimensional structure capable of immunospecifically binding to an epitope of an antigen. Thus, in embodiments, an ABD comprises a hypervariable region, optionally a VH and/or VL domain of an antibody, optionally at least a VH domain. In embodiments, an ABD comprises at least one complementarity determining region (CDR) of an antibody. In embodiments, an ABD comprises at least two CDRs of an antibody. In embodiments, an ABD comprises at least three CDRs of an antibody. In embodiments, an ABD comprises at least four CDRs of an antibody. In embodiments, an ABD comprises at least five CDRs of an antibody. In embodiments, an ABD comprises six CDRs of an antibody.

### 1. 3E10 Antibodies

In embodiments, the present disclosure relates to the use of 3E10 antibodies and antigen binding fragments thereof, e.g., for delivering therapeutic agents (e.g., oligonucleotides) into a cell within a subject. Although generally referred to herein as “3E10,” “3E10 antibodies,” and the like, it will be appreciated that disclosure herein referring to such antibodies also encompass antigen-binding fragments thereof, e.g., scFv, di-scFv, tr-scFv, regardless of whether it is specifically recited in each instance. Thus, when describing a feature of a 3E10 antibody for use in the various compositions, conjugates, fusion proteins, and methods disclosed herein, that same feature is implicitly disclosed with respect to 3E10 antigen-binding fragments as well.

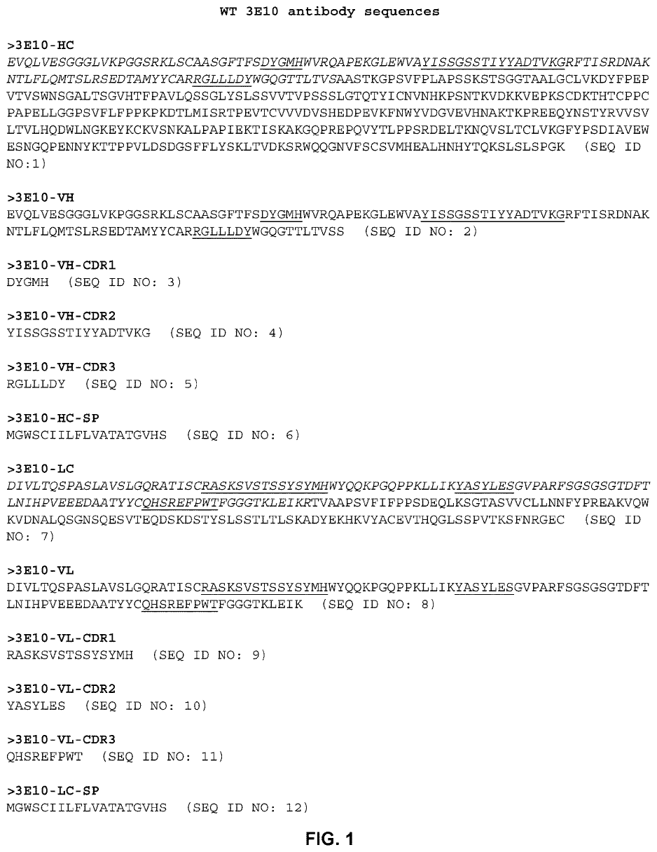

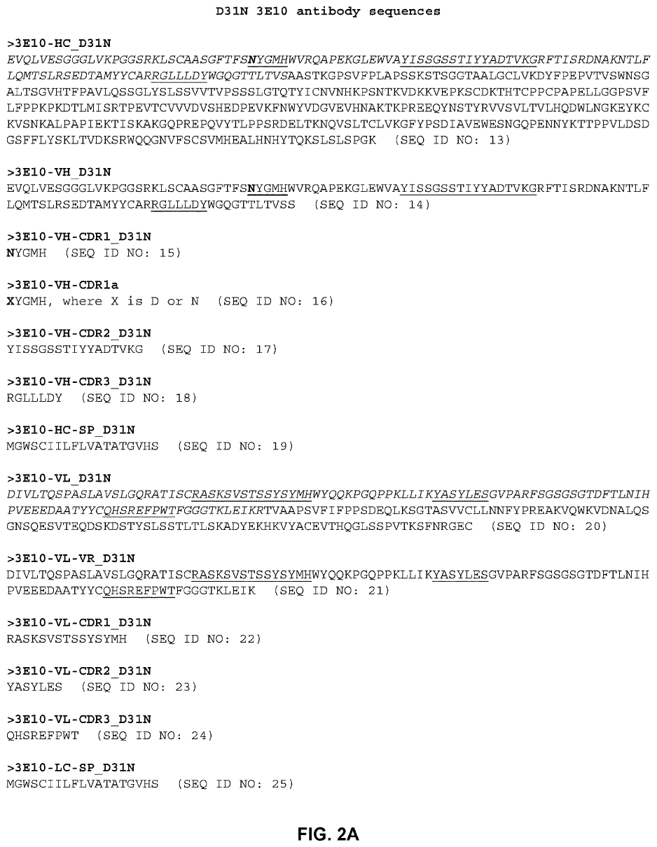

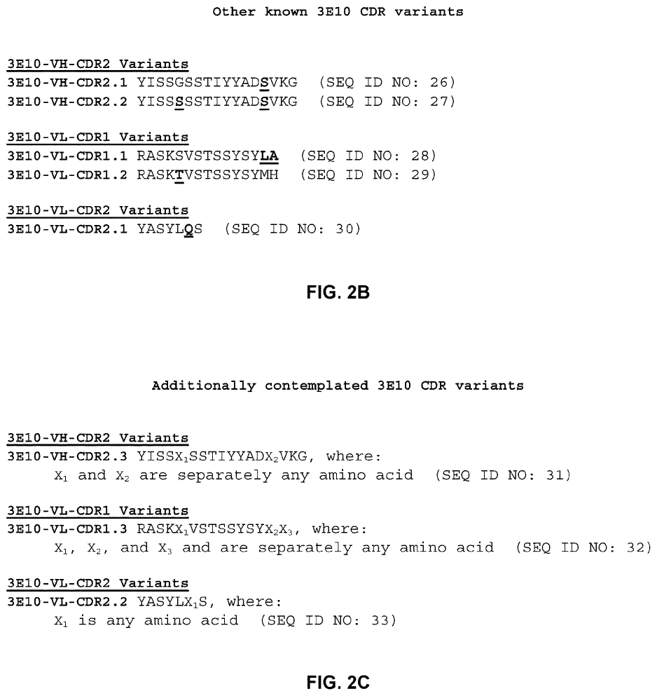

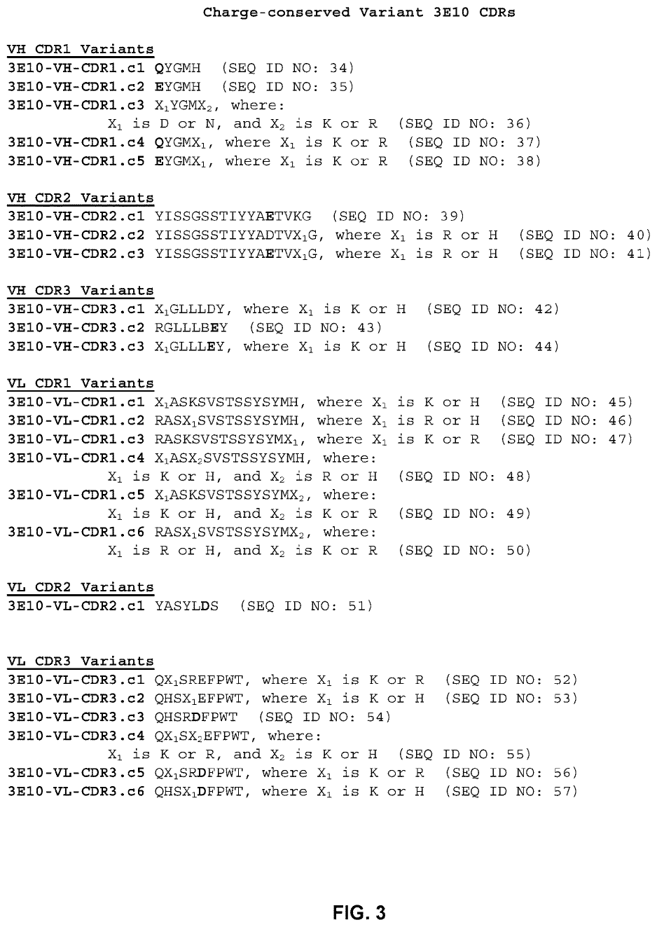

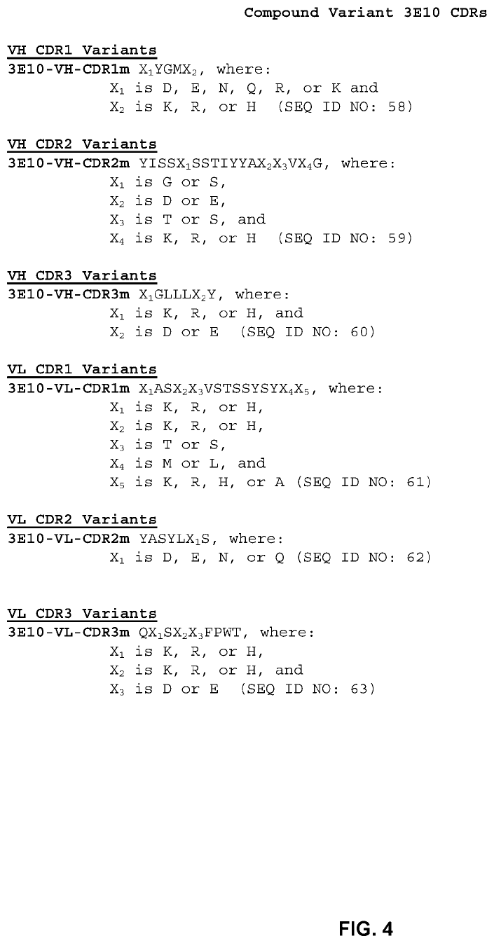

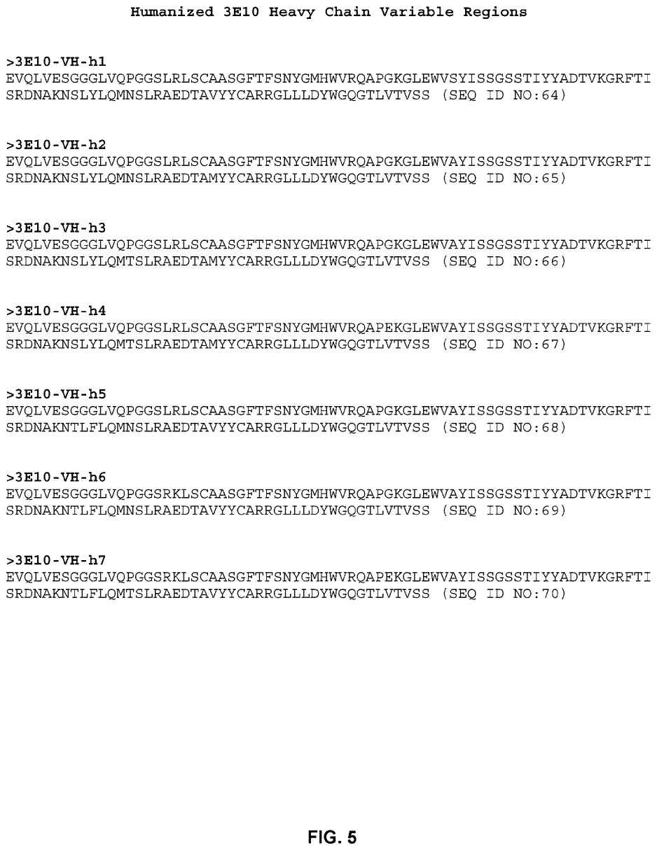

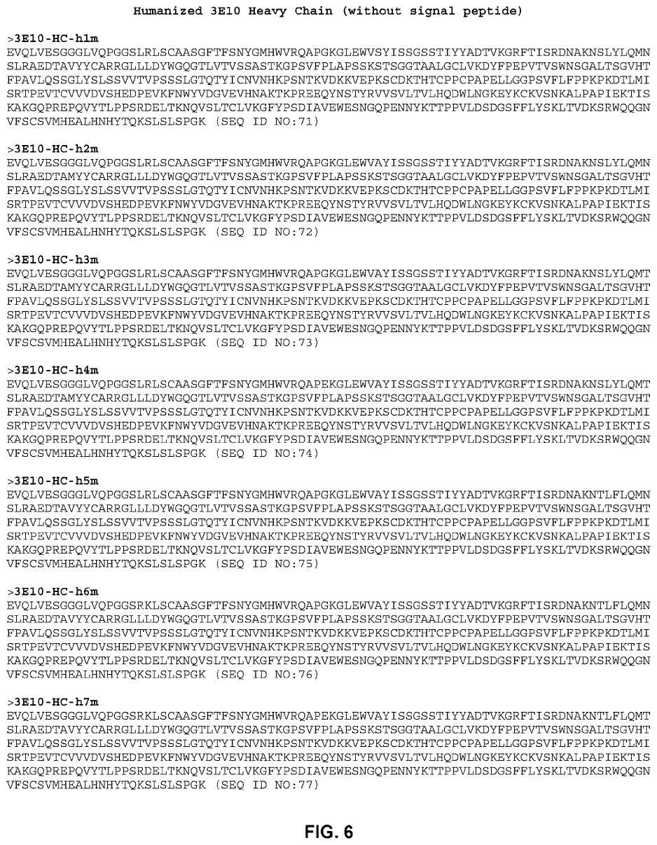

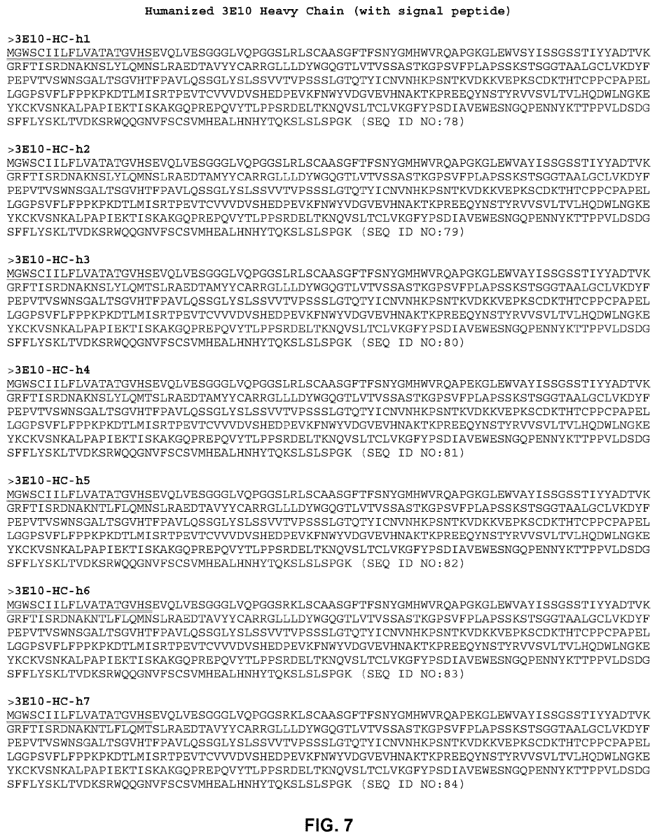

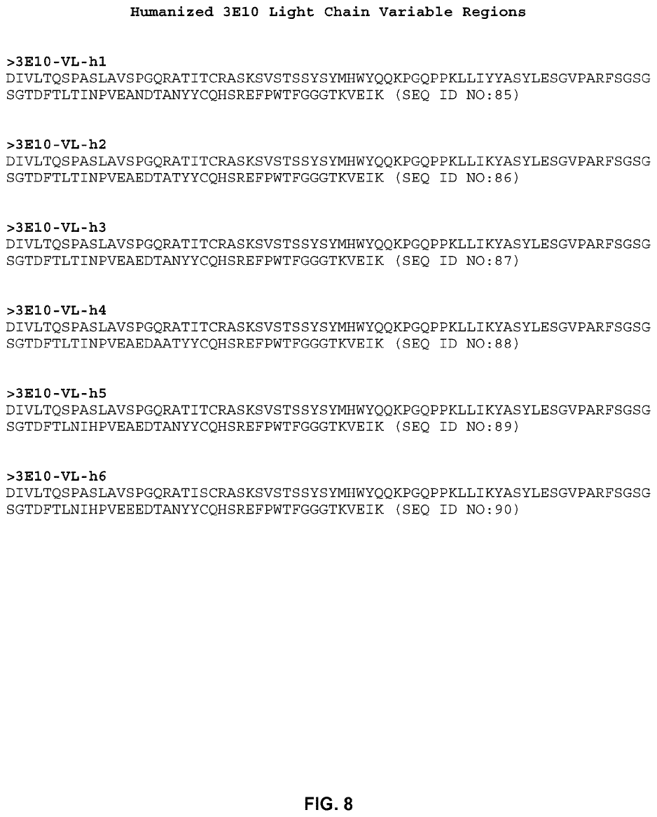

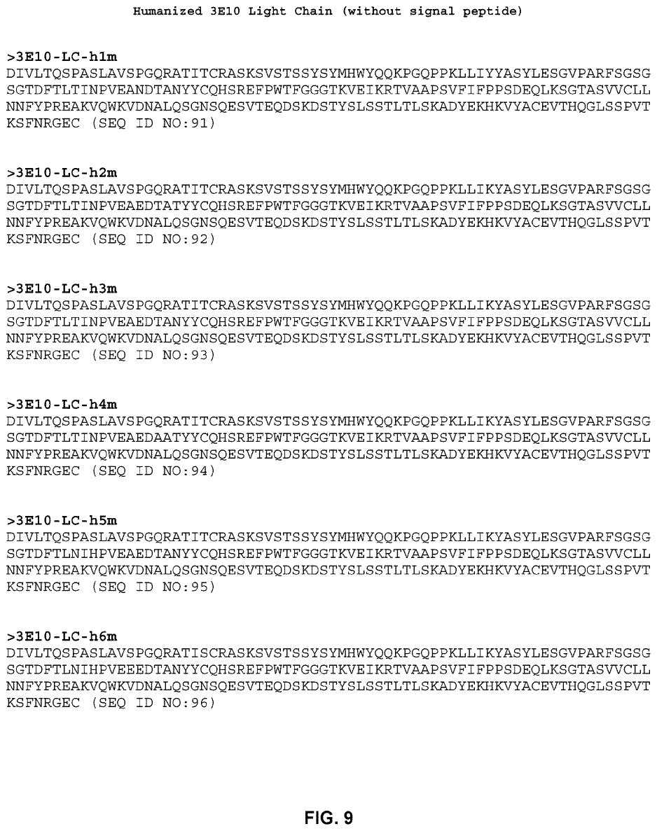

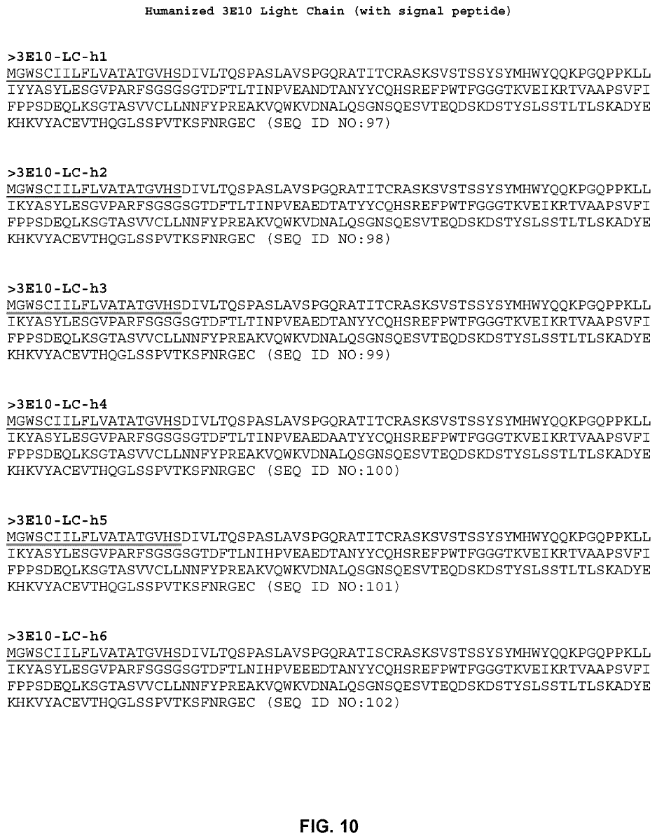

In embodiments, a 3E10 antibody comprises VL CDRs of SEQ ID NOs: 61, 62, and 63 and VH CDRs of SEQ ID NOs: 58, 59, and 60. In embodiments, a 3E10 antibody comprises VL CDRs of SEQ ID NOs: 9, 10, and 11 and VH CDRs of SEQ ID NOs: 3, 4, and 5. In embodiments, a 3E10 antibody comprises VL CDRs of SEQ ID NOs: 22, 23, and 24 and VH CDRs of SEQ ID NOs: 15, 17, and 18. In embodiments, a 3E10 antibody comprises VL CDRs of SEQ ID NOs: 9, 10, and 11 and VH CDRs of SEQ ID NOs: 16, 4, and 5. Other examples of 3E10 VL and VH CDR sequences are shown in FIGS. 1-4.

3E10 is known to interact with the ENT2 nucleoside transporter expressed on various cell types, including muscle cells and cancer cells. In fact, ENT2 is overexpressed in most, if not all cancers. Accordingly, an AOC provided herein can widely target cancers based on cell surface expression of ENT2 on cancer cells. Advantageously, an AOC described herein can target ENT2 and extracellular DNA simultaneously. Importantly, 3E10 has been shown to preferentially localize into tumor cell nuclei in vivo, likely due to increased DNA in the local environment released from ischemic and necrotic regions of tumor. Targeting of the 3E10 antibody to extracellular DNA is described in, for example in Weisbart, Sci Reports, 2015, which is herein incorporated by reference. By targeting ENT2 as well as extracellular DNA, an 3E10 AOC described herein presents a platform to target a variety of cancers and deliver therapeutic oligonucleotides to target and kill cancer cells.

In some aspects of the present disclosure, the antibody or antigen-binding fragment thereof is a murine, chimeric, humanized, or human antibody or antigen-binding fragment thereof.

In some aspects, an AOC of the present disclosure penetrates into cells and nuclei in an ENT2-dependent manner.

In embodiments, a second polynucleotide is non-covalently bound to an AOC of the present disclosure, to help facilitate cellular internalization of the AOC. That is, in some embodiments, polynucleotides conjugated to the antibody (cargo polynucleotides) do not interact with the nucleic acid-binding paratope of the antibody, and a second polynucleotide (e.g., carrier nucleic acid) is non-covalently complexed with the paratope to help facilitate internalization. In embodiments, the second polynucleotide is precomplexed with the AOC prior to administering the AOC to a subject. In embodiments, the second polynucleotide is an extracellular polynucleotide that is bound by the AOC at a site of interest in vivo, for example, at a site of tumor ischemia and/or necrosis. In embodiments, the second polynucleotide is DNA. In embodiments, the second polynucleotide is RNA.

In embodiments, an AOC disclosed herein comprises a VH and VL domain of a 3E10 antibody. In embodiments, the AOC comprises a VH CDR1, VH CDR2, VH CDR3, VL CDR1, VL CDR2, and/or VL CDR3 of a 3E10 antibody.

In some aspects, the present disclosure provides an antibody-oligonucleotide conjugate having the formula A-(L-Pᵣ)_{q}, wherein: A is a 3E10 antibody or antigen-binding fragment thereof, L is a linker, and P is an oligonucleotide as described herein, wherein the linker L links A to P. In embodiments, L is a cleavable linker and P is an siRNA molecule that targets cMyc mRNA. In some aspects of the present disclosure, the amino acid residue corresponding with D31 of the heavy chain CDR1 of the 3E10 antibody or antigen-binding fragment thereof is substituted with N. It is known in the art that mutation of aspartic acid at residue 31 of VH CDR1 to asparagine increases the cationic charge of this residue and enhanced nucleic acid binding and delivery in vivo (3E10-D31N). In embodiments, additional 3E10 antibody variants include mutation of aspartic acid at residue 31 of VH CDR1 to arginine (3E10-D31R), which modeling indicates expands cationic charge, or lysine (3E10-D31K) which modeling indicates changes charge orientation. Thus, in some aspects, the 3E10 antibody or antigen-binding fragment thereof includes a D31R or D31K substitution. In embodiments, additional 3E10 antibody variants include R96N, and/or S30D, alone or in combination with D31N, D31R, or D31K. All of the sequences disclosed herein having the residue corresponding to 3E10 D31 or N31, are expressly disclosed with a D31R or D31K or N31R or N31K substitution.

In embodiments, the present disclosure provides an antibody-oligonucleotide conjugate (AOC) having the formula A-(L-Pᵣ)_{q}, wherein: A is a 3E10 antibody or antigen-binding fragment thereof, L is a linker, P is an oligonucleotide as described herein, r is an integer from 1 to 4, and q is an integer from 1 to 16, wherein the linker L links A to (P); wherein the 3E10 antibody or antigen-binding fragment thereof comprises a heavy chain variable region (VH) CDR1 comprising the amino acid sequence of X1YGMX2, where X1 is D, E, N, Q, R, or K and X² is K, R, or H (SEQ ID NO:58). In embodiments, the antibody or antigen-binding fragment thereof comprises (a) a light chain variable region (VL) complementarity determining region (CDR) 1 comprising the amino acid sequence of X1ASX2X3VSTSSYSYX4X⁵, where X¹ is K, R, or H, X2 is K, R, or H, X3 is T or S, X4 is M or L, and X5 is K, R, H, or A (SEQ ID NO:61), (b) a VL CDR2 comprising the amino acid sequence of YASYLXIS, where X¹ is D, E, N, or Q (SEQ ID NO: 62), and (c) a VL CDR3 comprising the amino acid sequence of QX1SX2X3FPWT, where X1 is K, R, or H, X2 is K, R, or H, and X3 is D or E (SEQ ID NO:63), and (d) a heavy chain variable region (VH) CDR1 comprising the amino acid sequence of X1YGMX2, where X1 is D, E, N, Q, R, or K and X2 is K, R, or H (SEQ ID NO:58), (e) a VH CDR2 comprising the amino acid sequence of YISSX1SSTIYYAX2X3VX4G, where X1 is G or S, X2 is D or E, X3 is T or S, and X4 is K, R, or H (SEQ ID NO:59), and (f) a VH CDR3 comprising the amino acid sequence of X1GLLLX2Y, where X1 is K, R, or H, and X2 is D or E (SEQ ID NO:60).

In embodiments, the present disclosure provides an antibody-oligonucleotide conjugate (AOC) having the formula A-(L-Pᵣ)_{q}, wherein: A is a 3E10 antibody or antigen-binding fragment thereof, L is a linker, P is a payload as described herein, r is an integer from 1 to 4, and q is an integer from 1 to 16, wherein the linker L links A to P; wherein the 3E10 antibody or antigen-binding fragment thereof comprises a heavy chain variable region (VH) CDR1 comprising the amino acid sequence of NYGMH (SEQ ID NO: 15). In embodiments, the antibody or antigen-binding fragment thereof comprises (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO: 10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5). In embodiments, the antibody or antigen-binding fragment thereof comprises a light chain variable region (VL) comprising an amino acid sequence that is identical to SEQ ID NO:21. In embodiments, the antibody or antigen-binding fragment thereof comprises a heavy chain variable region (VH) comprising an amino acid sequence that is identical to SEQ ID NO:14. In embodiments, the antibody or antigen-binding fragment thereof comprises a full length light chain (LC) comprising an amino acid sequence that is identical to SEQ ID NO:20. In embodiments, the antibody or antigen-binding fragment thereof comprises a full length heavy chain (HC) comprising an amino acid sequence that is identical to SEQ ID NO: 13.

In embodiments, the antibody or antigen-binding fragment thereof comprises a light chain variable region (VL) comprising an amino acid sequence that is at least about 80%, at least about 85%, at least about 90%, at least about 95%, at least about 96%, at least about 97%, at least about 98%, or at least about 99% identical to SEQ ID NO:21. In embodiments, the antibody or antigen-binding fragment thereof comprises a heavy chain variable region (VH) comprising an amino acid sequence that is at least about 80%, at least about 85%, at least about 90%, at least about 95%, at least about 96%, at least about 97%, at least about 98%, or at least about 99% identical to SEQ ID NO:14. In embodiments, the antibody or antigen-binding fragment thereof comprises a full length light chain (LC) comprising an amino acid sequence that is at least about 80%, at least about 85%, at least about 90%, at least about 95%, at least about 96%, at least about 97%, at least about 98%, or at least about 99% identical to SEQ ID NO:20. In embodiments, the antibody or antigen-binding fragment thereof comprises a full length heavy chain (HC) comprising an amino acid sequence that is at least about 80%, at least about 85%, at least about 90%, at least about 95%, at least about 96%, at least about 97%, at least about 98%, or at least about 99% identical to SEQ ID NO:13.

In some aspects, the 3E10 antibody or antigen-binding fragment thereof can be transported into the cytoplasm and/or nucleus of the cells without the aid of a carrier or conjugate. For example, a monoclonal 3E10 antibody and active fragments thereof that are transported in vivo to the nucleus of mammalian cells without cytotoxic effect are disclosed in U.S. Pat. Nos. 4,812,397 and 7,189,396 to Richard Weisbart, the disclosures of which are incorporated by reference herein, in their entireties.

Amino acid sequences of 3E10 monoclonal antibodies and antigen-binding fragments thereof are known in the art. Example sequences of 3E10 heavy and light chains are provided below herein.

A murine version of the 3E10 antibody is described in Zack, et al., Immunology and Cell Biology, 72:513-520 (1994), the disclosure of which is incorporated by reference herein, in its entirety.

Amino acid variants of the 3E10 antibody are also known in the art, for example, as described in Zack, et al., J. Immunol., 157 (5): 2082-8 (1996). For example, amino acid position 31, in CDR1 of the heavy chain variable region of 3E10, influences nucleic acid binding and the antibody's ability to penetrate nuclei. Substitution of the ‘wild-type’ (e.g., relative to the original murine antibody) aspartic acid by asparagine (the ‘D31N’ mutation) improves nucleic acid binding and nuclei penetration of the antibody, relative to the ‘wild type’ murine antibody. See, for example, Zack, et al., Immunology and Cell Biology, 72:513-520 (1994); Weisbart, et al., J. Autoimmun., 11, 539-546 (1998); and Weisbart, Int. J. Oncol., 25, 1867-1873 (2004) (which are incorporated by reference herein, in their entireties).

Sequences for 3E10 antibodies and antigen-binding fragments or variants thereof, with the D31N substitution, are disclosed herein. In some aspects, the 3E10 antibodies and antigen-binding fragments thereof disclosed herein include the D31N substitution. In some aspects, other amino acids are substituted at position 31 in the 3E10 antibodies and antigen-binding fragments thereof disclosed herein. For example, D31R, D31K, or D31R substitutions are incorporated in some aspects of the present disclosure.

Other 3E10 light chain sequences are known in the art. See, for example, Zack, et al., J. Immunol., 15; 154 (4): 1987-94 (1995); GenBank: L16981.1-Mouse Ig rearranged L-chain gene, partial cds; GenBank: AAA65681.1-immunoglobulin light chain, partial [Mus musculus]).

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. In embodiments, an antibody disclosed herein is an IgA, IgD, IgE, IgG, or IgM antibody, including any subtype or isotype thereof. In embodiments, an antibody disclosed herein is based on the IgG class. In embodiments, an antibody disclosed herein is based on one of the subclasses of IgG, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M), and in embodiments, antibodies disclosed herein are based on IgG1 having D or E at position 356 and/or L or M at position 358.

The light chain generally comprises two domains, the variable light domain (containing the light chain CDRs and together with the variable heavy domains forming the Fv region), and a constant light chain region (often referred to as CL or CK). The heavy chain comprises a variable heavy domain and a constant domain, which includes a CH1-optional hinge-Fc domain comprising a CH₂-CH₃.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g., residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs useful for the compositions, conjugates, and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the VH CDRs (e.g., VH CDR1, VH CDR2 and VH CDR3) and the disclosure of each variable light region is a disclosure of the VL CDRs (e.g., VL CDR1, VL CDR2 and VL CDR3).

Throughout the present disclosure, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)). In some aspects, the present specification uses the IMGT system to define the complementarity determining regions (CDRs) provided herein.

The present disclosure provides a large number of different CDR sets. In this case, a “full CDR set” comprises the three variable light CDRs, e.g., a VL CDR1, VL CDR2, and VL CDR3, and the three variable heavy CDRs, e.g. VH CDR1, VH CDR2, and VH CDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As noted above herein, the present disclosure refers to different antibody domains of a 3E10 antibody or antigen-binding fragment thereof. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy (VH) domain, the variable light (VL) domain, the light constant domain, Fab domains and scFv domains.

### 2. Humanized Antibodies

In certain aspects, the antibodies of the disclosure comprise a heavy chain variable region from a particular germline heavy chain immunoglobulin gene and/or a light chain variable region from a particular germline light chain immunoglobulin gene. For example, such antibodies can comprise or consist of murine, chimeric, humanized, or human antibodies or antigen-binding fragments thereof comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence can contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody can be at least 95%, at least 96%, at least 97%, at least 98%, or at least 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody can display no more than 5, or even no more than 4, 3, 2, or 1 amino acid difference from the amino acid sequence encoded by the germline immunoglobulin gene.

In one aspect, the parent antibody has been affinity matured, as is known in the art. Structure-based methods can be employed for humanization and affinity maturation, for example, as U.S. Patent Publication No. 2006/0008883, which is incorporated herein by reference. Selection based methods can be employed to humanize and/or affinity mature antibody variable regions, including but not limited to methods described in Wu et al., 1999, J. Mol. Biol. 294:151-162; Baca et al., 1997, J. Biol. Chem. 272 (16): 10678-10684; Rosok et al., 1996, J. Biol. Chem. 271 (37): 22611-22618; Rader et al., 1998, Proc. Natl. Acad. Sci. USA 95:8910-8915; Krauss et al., 2003, Protein Engineering 16 (10): 753-759, all of which are incorporated herein by reference. Other humanization methods can involve the grafting of only parts of the CDRs, including but not limited to methods described in U.S. Patent Publication No. 2001/0035606; Tan et al., 2002, J. Immunol. 169:1119-1125; De Pascalis et al., 2002, J. Immunol. 169:3076-3084, all of which are incorporated herein by reference.

### 3. Fc Variants

In embodiments, one or more amino acid modifications can be introduced into the Fc region of an antibody provided herein, thereby generating an Fc region variant. The Fc region variant can comprise a human Fc region sequence (e.g., a human IgG1, IgG2, IgG3 or IgG4 Fc region) comprising an amino acid modification (e.g. a substitution) at one or more amino acid positions.

In embodiments, an Fc region variant possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays known in the art can be conducted to ensure that the antibody lacks FcγR binding (hence likely lacking ADCC activity), but retains FcRn binding ability. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18 (12): 1759-1769 (2006)).

In embodiments, an antibody provided herein can have reduced effector function and thus can comprise a substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In embodiments, an Fc region variant provided herein can have improved or diminished binding to FcRs. See, e.g., U.S. Pat. No. 6,737,056; WO 2004/056312, and Shields et al., J. Biol. Chem. 9 (2): 6591-6604 (2001), the disclosure of which are incorporated herein by reference, in their entireties.

In embodiments, an Fc region variant provided herein comprises an Fc region with one or more amino acid substitutions which improve ADCC, e.g., substitutions at positions 298, 333, and/or 334 of the Fc region (EU numbering of residues).

In embodiments, an Fc region variant provided herein comprises alterations that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164:4178-4184 (2000).

In embodiments, an Fc region variant provided herein comprises alterations that result in increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), e.g., as described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

In embodiments, an Fc region variant provided herein comprises “knob-in-hole” or “skew” variants, which refer to amino acid engineering that creates stearic influences to favor heterodimeric formation and disfavor homodimeric formation, as described in U.S. Ser. No. 61/596,846, Ridgway et al, Protein Engineering 9 (7): 617 (1996); Atwell et al, J. Mol. Biol. 1997 270:26; U.S. Pat. No. 8,216,805, all of which are hereby incorporated by reference in their entirety.

In embodiments, an Fc region variant provided herein comprises alterations described in Duncan & Winter, Nature 322:738-40 (1988); U.S. Pat. Nos. 5,648,260; 5,624,821; and WO 94/29351.

### 4. Antibody Fragments

In embodiments, the antibody portion of an AOC described herein comprises an antigen-binding fragment of a 3E10 antibody. In embodiments, the antigen-binding fragment retains the desired biological activity of a 3E10 antibody. In embodiments, the antigen-binding fragment retains at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% of the desired biological activity of a 3E10 antibody. In embodiments, the antigen-binding fragment retains the ability of the antibody to bind to its target antigen, e.g., a nucleic acid, e.g., DNA. In embodiments, the antigen-binding fragment retains the ability of the antibody to bind to a cellular receptor, e.g., ENT2. In embodiments, the antigen-binding fragment retains the ability of the antibody to be internalized by a target cell.

In embodiments, the 3E10 antibody or antigen binding fragment thereof comprises a single-chain fragment variable (scFv), a tandem double scFv, an (scFv)₂, a minibody, a VHH, an scFv-Fc, a CrossMab, a dual variable domain immunoglobulin (DVD-Ig), a single-chain tandem fragment variable (scTaFv), a diabody, a tandem diabody (TandAb), a Fabsc, a modular IgG-scFv, a Fab, or an F(ab′)₂.

In embodiments, an antigen-binding fragment of a 3E10 antibody or antigen-binding fragment thereof comprises a CrossMab. In the CrossMab format, complementary mutations are introduced in the heavy chain constant region of each arm to generate so-called “holes and knobs,” resulting in preferred association between different arms, forming a heterodimer, rather than a homodimer of two of the same arms. The exact residues that are mutated in the heavy chain constant region of a CrossMab bispecific antibody to form “holes” and “knobs” can vary depending on the specific design and optimization goals of the antibody. For more information on CrossMab antibodies see, for example, Huang, J., et al., Journal of Biological Chemistry, 294 (50): 19001-10 (2019), the disclosure of which is incorporated herein by reference in its entirety.

In embodiments, an antigen-binding fragment of a 3E10 antibody or antigen-binding fragment thereof comprises a divalent, dual-variable domain immunoglobulin (DVD-Ig) of a 3E10 antibody or antigen binding fragment thereof. In the DVD-Ig format, each arm of the antibody contains two VH/VL pairs. In some embodiments, one of the VH/VL pairs comprises 3E10 VH and VL CDRs. For more information on DVD-Ig antibodies see, for example, Polson A G, et al., Journal of immunotherapy. 29 (3): 241-50 (2006) and U.S. Pat. No. 7,612,181, the disclosures of which are incorporated herein by reference in their entireties.

In embodiments, an antigen-binding fragment of a 3E10 antibody or antigen-binding fragment thereof comprises a single-chain variable fragment (scFv). “Single chain Fv” or “scFv,” as used herein, refers to a VH domain covalently attached to a VL domain through a linker, e.g., a scFv linker as discussed herein, to form a continuous protein chain. A scFv domain can be in either arrangement from N- to C-terminus (i.e., VH-linker-VL or VL-linker-VH). In the sequences depicted in the sequence listing and in the figures herein, the order of the VH and VL domain is indicated in the name, e.g., H.X_L.Y means the N- to C-terminus arrangement is VH-linker-VL, and L.Y_H.X means the N- to C-terminus arrangement is VL-linker-VH.

In embodiments, an antigen-binding fragment of a 3E10 antibody or antigen-binding fragment thereof comprises a tandem double scFv. A tandem double scFv has two scFv domains linked in a linear fashion. In some embodiments, each scFv domain is derived from a different antibody and provides independent antigen-binding specificity. In some embodiments, one of the scFv domains comprises 3E10 VH and VL CDRs. For more information on tandem double scFvs see, for example, Bossen C, et al., MAbs, 4 (2): 200-08 (2012), the disclosure of which is incorporated herein by reference in its entirety.

In embodiments, an antigen-binding fragment of a 3E10 antibody or antigen-binding fragment thereof comprises a dimeric scFv antibody (scFv)₂. A dimeric scFv antibody has two scFv domains linked in a dimeric arrangement. In some embodiments, each scFv domain is derived from a different antibody and provides independent antigen-binding specificity. In some embodiments, one of the scFv domains comprises 3E10 VH and VL CDRs. For more information on dimeric scFv antibodies see, for example, Llewellyn C, et al., Journal of immunological methods, 273 (1-2): 33-44 (2002), the disclosure of which is incorporated herein by reference in its entirety.

In embodiments, an antigen-binding fragment of a 3E10 antibody or antigen-binding fragment thereof comprises a scFv-Fc. An “scFv-Fc,” as meant herein, is a polypeptide that consists of a heavy and a light chain variable region of an antibody joined by a linker, which is followed by an Fc polypeptide chain of an antibody, optionally the Fc region of a human IgG antibody, such as an IgG1, IgG2, IgG3, or IgG4 antibody.

In embodiments, an antigen-binding fragment of a 3E10 antibody or antigen-binding fragment thereof comprises a single-chain tandem fragment variable (scTaFv) antibody. A single-chain tandem fragment variable (scTaFv) antibody is a type of bispecific antibody that consists of two variable fragment (VH and VL) domains linked in a tandem arrangement. In some embodiments, one of the variable fragment domains comprises 3E10 VH and VL CDRs. For more information on scTaFv antibodies see, for example, Schramm C, et al., MAbs 5 (3): 442-49 (2013), the disclosure of which is incorporated herein by reference in its entirety.

In embodiments, an antigen-binding fragment of a 3E10 antibody or antigen-binding fragment thereof comprises a VHH, also referred to as a nanobody. As used herein, the term “VHH” refers to a variable domain of heavy chain of heavy-chain antibody. A VHH is a molecule that can recognize an antigen through a single domain and is the smallest unit among antibody molecules that have been found to date. In embodiments, a VHH can include one or more variable domains of heavy chain derived from a heavy-chain antibody, and the number of the variable domains of heavy chain included in the VHH is not limited.

In embodiments, an antigen-binding fragment of a 3E10 antibody or antigen-binding fragment thereof comprises a diabody. As used herein, “diabody” refers to a divalent antibody comprising two polypeptide chains, wherein each polypeptide chain is too short for a pair to form between two domains on the same chain such that each domain is paired with a complementary domain on another polypeptide chain (see, e.g., Holliger et al., 1993, Proc. Natl. Acad. Sci. USA 90:6444-48 and Poljak et al., 1994, Structure 2:1121-23). If the two polypeptide chains of the diabody are identical, there will be two identical antigen-binding sites in the diabody resulting from their pairing. In embodiments, one of the antigen binding domains of the diabody comprises 3E10 VH and VL CDRs. For more information on diabodies see, for example, Hoogenboom H R, et al., Trends Biotechnol., 21(12): 553-57 (2003), the disclosure of which is incorporated herein by reference in its entirety. Polypeptide chains of different sequences can be used to prepare diabodies with two different antigen-binding sites. Similarly, as used herein, “triabodies” and “tetrabodies” refer to antibodies that contain three and four polypeptide chains, respectively, and form three and four antigen-binding sites (which can be the same or different), respectively.

The term “minibody” is used to refer to an scFv-CH₃ fusion protein that self-assembles into a bivalent dimer of 80 kDa (ScFv-CH3)2.

In embodiments, an antigen-binding fragment of a 3E10 antibody or antigen-binding fragment thereof comprises a tandem diabody (TandAb). A tandem diabody has wo antigen-binding domains (VH and VL) linked in a tandem arrangement by a flexible peptide linker. In some embodiments, one of the antigen binding domains comprises 3E10 VH and VL CDRs. For more information on diabodies see, for example, Sidelmann J G, et al., Mol Immunol., 45 (9): 2597-607 (2008), the disclosure of which is incorporated herein by reference in its entirety.

In embodiments, an antigen-binding fragment of a 3E10 antibody or antigen-binding fragment thereof comprises a Fabsc. As used herein, a “Fabsc” format antibody molecule typically refers to a bispecific antibody molecule having a Fab fragment, which generally includes a hinge region, which is at the C-terminus of the Fab fragment linked to the N-terminus of a CH₂ domain, of which the C-terminus is in turn linked to the N-terminus of a scFv fragment.

In embodiments, an antigen-binding fragment of a 3E10 antibody or antigen-binding fragment thereof comprises a scFab. A scFab, also known as a single-chain fragment antigen binding (Fab), is a type of antibody fragment that combines the variable heavy chain (VH) and variable light chain (VL) domains into a single polypeptide chain, linked by a peptide linker. The domain structure of a Fabsc includes the variable domains of both the heavy chain and light chain (VH and VL), and a peptide linker that connects the two domains. In addition to the variable domains, a Fabsc also includes the constant domains of the light chain (CL) and the hinge region of the heavy chain. In some embodiments, one of the antigen binding domains comprises 3E10 VH and VL CDRs. For more information on Fabscs see, for example, Kettner, C., et al., Frontiers in Immunology, 8 (8): 453 (2017), the disclosure of which is incorporated herein by reference in its entirety.

In embodiments, an antigen-binding fragment of a 3E10 antibody or antigen-binding fragment thereof comprises an IgG-scFv. An IgG-scFv is an antibody in which a scFv is fused to the light chain or heavy chain of an IgG. In some embodiments, the scFv comprises 3E10 VH and VL CDRs. In some embodiments, the IgG comprises 3E10 VH and V LCDRs. In some embodiments, the antibody is an F(ab′)2.

### 5. Bispecific Antibodies

In embodiments, 3E10 antibodies and antigen-binding fragments thereof can be modified to improve their therapeutic potential. For example, in embodiments, the cell-penetrating anti-DNA antibody is conjugated to another antibody specific for a second therapeutic target in the cytoplasm and/or nucleus of a target cell. For example, in embodiments, the cell-penetrating 3E10 antibody is a bispecific antibody having a first heavy chain and a first light chain from 3E10 and a second heavy chain and a second light chain from a monoclonal antibody that specifically binds a second therapeutic target.

Bispecific antibodies and other binding proteins having a first heavy chain and a first light chain from 3E10 and a second heavy chain and a second light chain from a monoclonal antibody that specifically binds a second target are discussed in Weisbart, et al., Mol. Cancer Ther., 11(10): 2169-73 (2012), and Weisbart, et al., Int. J. Oncology, 25:1113-8 (2004), and U.S. Patent Application No. 2013/0266570, which are specifically incorporated by reference in their entireties. In embodiments, the second target is specific for a target cell-type, tissue, organ, etc. Thus the second heavy chain and second light chain can serve as a targeting moiety that targets the complex to the target cell-type, tissue, organ. In embodiments, the second heavy chain and second light chain target, hematopoietic stem cells, CD34⁺ cells, T cells or any another cell type of interest, e.g., by targeting a receptor or ligand expressed on the cell type of interest. In embodiments, the second heavy chain and second light chain target the thymus, spleen, or cancer cells.

Bispecific antibodies can be used to direct cytotoxic agents or drugs to cells which express a particular antigen. These antibodies possess two binding sites directed at two different antigens or two different epitopes on the same antigen. For example, in embodiments the bispecific can comprise one arm for ENT2 engagement and another arm for a second target. Bispecific antibody design can include a variety of antibody designs with multiple binding arms. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168). Antibodies with more than two valencies are also contemplated. For example, trispecific antibodies can be prepared (Tutt et al., J. Immunol. 147:60 (1991)). In embodiments the contemplated bispecific antibody disclosed herein can be conjugated as a bispecific AOC.

Heteroconjugate antibodies are also within the scope of the present disclosure. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate. In embodiments the contemplated heteroconjugate antibody disclosed herein can be conjugated as a heteroconjugate AOC.

### 6. 3E10 Sequences

In embodiments, a 3E10 antibody or antigen-binding fragment thereof described herein includes CDR sequences corresponding to the parent 3E10 antibody.

Accordingly, in embodiments, a 3E10 antibody or antigen-binding fragment thereof comprises (a) a light chain variable region (VL) complementarity determining region (CDR) 1 comprising the amino acid sequence of X1ASX2X3VSTSSYSYX4X5, where X1 is K, R, or H, X2 is K, R, or H, X3 is T or S, X4 is M or L, and X5 is K, R, H, or A (SEQ ID NO:61), (b) a VL CDR2 comprising the amino acid sequence of YASYLXIS, where X1 is D, E, N, or Q (SEQ ID NO:62), and (c) a VL CDR3 comprising the amino acid sequence of QX1SX2X3FPWT, where X1 is K, R, or H, X2 is K, R, or H, and X₃ is D or E (SEQ ID NO:63), and (d) a heavy chain variable region (VH) CDR1 comprising the amino acid sequence of X1YGMX2, where X1 is D, E, N, Q, R, or K and X2 is K, R, or H (SEQ ID NO:58), (e) a VH CDR2 comprising the amino acid sequence of YISSX1SSTIYYAX2X3VX4G, where X1 is G or S, X2 is D or E, X3 is T or S, and X4 is K, R, or H (SEQ ID NO:59), and (f) a VH CDR3 comprising the amino acid sequence of X1GLLLX2Y, where X1 is K, R, or H, and X2 is D or E (SEQ ID NO:60).

In embodiments, a 3E10 antibody or antigen-binding fragment thereof includes a light chain variable region (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1 (SEQ ID NO:3), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In embodiments, a 3E10 antibody or antigen-binding fragment thereof described herein includes CDR sequences from a variant 3E10 antibody that includes a D31N amino acid substitution in the VH CDR1. Accordingly, in embodiments, the a 3E10 antibody or antigen-binding fragment thereof includes a light chain variable region (VL) complementarity determining region (CDR) 1 comprising the amino acid sequence of 3E10-VL-CDR1_D31N (SEQ ID NO:22), a VL CDR2 comprising the amino acid sequence of 3E10-VL-CDR2_D31N (SEQ ID NO:23), a VL CDR3 comprising the amino acid sequence of 3E10-VL-CDR3_D31N (SEQ ID NO:24), a heavy chain variable region (VH) CDR1 comprising the amino acid sequence of 3E10-VH-CDR1_D31N (SEQ ID NO:15), a VH CDR2 comprising the amino acid sequence of 3E10-VH-CDR2_D31N (SEQ ID NO:17), and a VH CDR3 comprising the amino acid sequence of 3E10-VH-CDR3_D31N (SEQ ID NO:18).

In embodiments, a 3E10 antibody or antigen-binding fragment thereof described herein refers to CDR sequences corresponding to the parent 3E10 antibody, optionally including a D31N amino acid substitution in the VH CDR1. Accordingly, in embodiments, a 3E10 antibody or antigen-binding fragment thereof includes a light chain variable region (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable region (VH) CDR1 comprising the amino acid sequence of 3E10-VH-CDR1a (SEQ ID NO: 16), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In embodiments, a 3E10 antibody or antigen-binding fragment thereof described herein includes CDR sequences corresponding to the parent 3E10 antibody, with a known amino acid substitution in one or more CDR. Accordingly, in embodiments, a 3E10 antibody or antigen-binding fragment thereof described herein includes one or more amino acid substitution, relative to the CDR sequences of the parent 3E10 or 3E10-D31N variant, selected from a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in embodiments, a 3E10 antibody or antigen-binding fragment thereof includes VH CDR2 comprising the amino acid sequence of 3E10-VH-CDR2.1 (SEQ ID NO: 26) or 3E10-VH-CDR2.2 (SEQ ID NO:27). In embodiments, the 3E10 antibody or antigen-binding fragment thereof further includes VL CDRs 1-3, and VH CDRs 1 and 3 according to the parent 3E10 antibody. In embodiments, the 3E10 antibody or antigen-binding fragment thereof further includes VL CDRs 1-3, and VH CDRs 1 and 3 according to the 3E10-D31N variant. In embodiments, the 3E10 antibody or antigen-binding fragment thereof further includes VL CDRs 1-3, and VH CDRs 1 and 3 having one or more amino acid substitutions relative to the CDRs of the parent 3E10 antibody or relative to the 3E10-D31N variant.

In embodiments, a 3E10 antibody or antigen-binding fragment thereof includes VL CDR1 comprising the amino acid sequence of 3E10-VL-CDR1.1 (SEQ ID NO:28) or 3E10-VL-CDR1.2 (SEQ ID NO:29). In embodiments, the 3E10 antibody or antigen-binding fragment thereof further includes VL CDRs 2 and 3, and VH CDRs 1-3 according to the parent 3E10 antibody. In embodiments, the 3E10 antibody or antigen-binding fragment thereof further includes VL CDRs 2 and 3, and VH CDRs 1-3 according to the 3E10-D31N variant. In embodiments, the 3E10 antibody or antigen-binding fragment thereof further includes VL CDRs 2 and 3, and VH CDRs 1-3 having one or more amino acid substitutions relative to the CDRs of the parent 3E10 antibody or relative to the 3E10-D31N variant.

In embodiments, a 3E10 antibody or antigen-binding fragment thereof includes VL CDR2 comprising the amino acid sequence of 3E10-VL-CDR2.1 (SEQ ID NO:30). In embodiments, the 3E10 antibody or antigen-binding fragment thereof further includes VL CDRs 1 and 3, and VH CDRs 1-3 according to the parent 3E10 antibody. In embodiments, the 3E10 antibody or antigen-binding fragment thereof further includes VL CDRs 1 and 3, and VH CDRs 1-3 according to the 3E10-D31N variant. In embodiments, the 3E10 antibody or antigen-binding fragment thereof further includes VL CDRs 1 and 3, and VH CDRs 1-3 having one or more amino acid substitutions relative to the CDRs of the parent 3E10 antibody or relative to the 3E10-D31N variant.

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

In embodiments, a 3E10 antibody or antigen-binding fragment thereof includes VH CDR2 comprising the amino acid sequence of 3E10-VH-CDR2.3 (SEQ ID NO:31). In embodiments, the 3E10 antibody or antigen-binding fragment thereof further includes VL CDRs 1-3, and VH CDRs 1 and 3 according to the parent 3E10 antibody. In embodiments, the 3E10 antibody or antigen-binding fragment thereof further includes VL CDRs 1-3, and VH CDRs 1 and 3 according to the 3E10-D31N variant. In embodiments, the 3E10 antibody or antigen-binding fragment thereof further includes VL CDRs 1-3, and VH CDRs 1 and 3 having one or more amino acid substitutions relative to the CDRs of the parent 3E10 antibody or relative to the 3E10-D31N variant, e.g., as described herein.

In embodiments, a 3E10 antibody or antigen-binding fragment thereof includes VL CDR1 comprising the amino acid sequence of 3E10-VL-CDR1.3 (SEQ ID NO:32). In embodiments, the 3E10 antibody or antigen-binding fragment thereof further includes VL CDRs 2 and 3, and VH CDRs 1-3 according to the parent 3E10 antibody. In embodiments, the 3E10 antibody or antigen-binding fragment thereof further includes VL CDRs 2 and 3, and VH CDRs 1-3 according to the 3E10-D31N variant. In embodiments, the 3E10 antibody or antigen-binding fragment thereof further includes VL CDRs 2 and 3, and VH CDRs 1-3 having one or more amino acid substitutions relative to the CDRs of the parent 3E10 antibody or relative to the 3E10-D31N variant, e.g., as described herein.

In embodiments, a 3E10 antibody or antigen-binding fragment thereof, includes VL CDR2 comprising the amino acid sequence of 3E10-VL-CDR2.2 (SEQ ID NO:33). In embodiments, the 3E10 antibody or antigen-binding fragment thereof further includes VL CDRs 1 and 3, and VH CDRs 1-3 according to the parent 3E10 antibody. In embodiments, the 3E10 antibody or antigen-binding fragment thereof further includes VL CDRs 1 and 3, and VH CDRs 1-3 according to the 3E10-D31N variant. In embodiments, the 3E10 antibody or antigen-binding fragment thereof further includes VL CDRs 1 and 3, and VH CDRs 1-3 having one or more amino acid substitutions relative to the CDRs of the parent 3E10 antibody or relative to the 3E10-D31N variant, e.g., as described herein.

In embodiments, a 3E10 antibody or antigen-binding fragment thereof includes VH CDR1 comprising the amino acid sequence of 3E10-VH-CDR1.c1 (SEQ ID NO:34), 3E10-VH-CDR1.c2 (SEQ ID NO:35), 3E10-VH-CDR1.c3 (SEQ ID NO:36), 3E10-VH-CDR1.c4 (SEQ ID NO: 37), or 3E10-VH-CDR1.c5 (SEQ ID NO:38). In embodiments, the 3E10 antibody or antigen-binding fragment thereof further includes VL CDRs 1-3, and VH CDRs 2 and 3 according to the parent 3E10 antibody. In embodiments, the 3E10 antibody or antigen-binding fragment thereof further includes VL CDRs 1-3, and VH CDRs 2 and 3 having one or more amino acid substitutions relative to the CDRs of the parent 3E10 antibody, e.g., as described herein.

In embodiments, a 3E10 antibody or antigen-binding fragment thereof includes VH CDR2 comprising the amino acid sequence of 3E10-VH-CDR2.c1 (SEQ ID NO:39), 3E10-VH-CDR2.c2 (SEQ ID NO:40), or 3E10-VH-CDR2.c3 (SEQ ID NO:41). In embodiments, the 3E10 antibody or antigen-binding fragment thereof further includes VL CDRs 1-3, and VH CDRs 1 and 3 according to the parent 3E10 antibody. In embodiments, the 3E10 antibody or antigen-binding fragment thereof further includes VL CDRs 1-3, and VH CDRs 1 and 3 according to the 3E10-D31N variant. In embodiments, the 3E10 antibody or antigen-binding fragment thereof further includes VL CDRs 1-3, and VH CDRs 1 and 3 having one or more amino acid substitutions relative to the CDRs of the parent 3E10 antibody, e.g., as described herein.

In embodiments, a 3E10 antibody or antigen-binding fragment thereof includes VH CDR3 comprising the amino acid sequence of 3E10-VH-CDR3.c1 (SEQ ID NO:42), 3E10-VH-CDR3.c2 (SEQ ID NO:43), or 3E10-VH-CDR3.c3 (SEQ ID NO:44). In embodiments, the 3E10 antibody or antigen-binding fragment thereof further includes VL CDRs 1-3, and VH CDRs 1 and 2 according to the parent 3E10 antibody. In embodiments, the 3E10 antibody or antigen-binding fragment thereof further includes VL CDRs 1-3, and VH CDRs 1 and 2 according to the 3E10-D31N variant. In embodiments, the 3E10 antibody or antigen-binding fragment thereof further includes VL CDRs 1-3, and VH CDRs 1 and 2 having one or more amino acid substitutions relative to the CDRs of the parent 3E10 antibody, e.g., as described herein.

In embodiments, a 3E10 antibody or antigen-binding fragment thereof includes VL CDR1 comprising the amino acid sequence of 3E10-VL-CDR1.c1 (SEQ ID NO:45), 3E10-VL-CDR1.c2 (SEQ ID NO:46), 3E10-VL-CDR1.c3 (SEQ ID NO:47), 3E10-VL-CDR1.c4 (SEQ ID NO: 48), 3E10-VL-CDR1.c5 (SEQ ID NO:49), or 3E10-VL-CDR1.c6 (SEQ ID NO:50). In embodiments, the 3E10 antibody or antigen-binding fragment thereof further includes VL CDRs 2 and 3, and VH CDRs 1-3 according to the parent 3E10 antibody. In embodiments, the 3E10 antibody or antigen-binding fragment thereof further includes VL CDRs 2 and 3, and VH CDRs 1-3 according to the 3E10-D31N variant. In embodiments, the 3E10 antibody or antigen-binding fragment thereof further includes VL CDRs 2 and 3, and VH CDRs 1-3 having one or more amino acid substitutions relative to the CDRs of the parent 3E10 antibody, e.g., as described herein.

In embodiments, a 3E10 antibody or antigen-binding fragment thereof includes VL CDR2 comprising the amino acid sequence of 3E10-VL-CDR2.c1 (SEQ ID NO:51). In embodiments, the 3E10 antibody or antigen-binding fragment thereof further includes VL CDRs 1 and 3, and VH CDRs 1-3 according to the parent 3E10 antibody. In embodiments, the 3E10 antibody or antigen-binding fragment thereof further includes VL CDRs 1 and 3, and VH CDRs 1-3 according to the 3E10-D31N variant. In embodiments, the 3E10 antibody or antigen-binding fragment thereof further includes VL CDRs 1 and 3, and VH CDRs 1-3 having one or more amino acid substitutions relative to the CDRs of the parent 3E10 antibody, e.g., as described herein.

In embodiments, a 3E10 antibody or antigen-binding fragment thereof includes VL CDR3 comprising the amino acid sequence of 3E10-VL-CDR3.c1 (SEQ ID NO:52), 3E10-VL-CDR3.c2 (SEQ ID NO:53), 3E10-VL-CDR3.c3 (SEQ ID NO:54), 3E10-VL-CDR3.c4 (SEQ ID NO: 55), 3E10-VL-CDR3.c5 (SEQ ID NO:56), or 3E10-VL-CDR3.c6 (SEQ ID NO:57). In embodiments, the 3E10 antibody or antigen-binding fragment thereof further includes VL CDRs 1 and 2, and VH CDRs 1-3 according to the parent 3E10 antibody. In embodiments, the 3E10 antibody or antigen-binding fragment thereof further includes VL CDRs 1 and 2, and VH CDRs 1-3 according to the 3E10-D31N variant. In embodiments, the 3E10 antibody or antigen-binding fragment thereof further includes VL CDRs 1 and 2, and VH CDRs 1-3 having one or more amino acid substitutions relative to the CDRs of the parent 3E10 antibody, e.g., as described herein.

It is also contemplated that a 3E10 antibody or antigen-binding fragment thereof, as described herein, includes any combination of the 3E10 CDR amino acid substitutions described above.

Accordingly, in embodiments, a 3E10 antibody or antigen-binding fragment thereof includes VH CDR1 comprising the amino acid sequence of 3E10-VH-CDR1m (SEQ ID NO: 58). In embodiments, the 3E10 antibody or antigen-binding fragment thereof further includes VL CDRs 1-3, and VH CDRs 2 and 3 according to the parent 3E10 antibody. In embodiments, the 3E10 antibody or antigen-binding fragment thereof further includes VL CDRs 1-3, and VH CDRs 2 and 3 having one or more amino acid substitutions relative to the CDRs of the parent 3E10 antibody, e.g., as described herein.

In embodiments, a 3E10 antibody or antigen-binding fragment thereof includes VH CDR2 comprising the amino acid sequence of 3E10-VH-CDR2m (SEQ ID NO:59). In embodiments, the 3E10 antibody or antigen-binding fragment thereof further includes VL CDRs 1-3, and VH CDRs 1 and 3 according to the parent 3E10 antibody. In embodiments, the 3E10 antibody or antigen-binding fragment thereof further includes VL CDRs 1-3, and VH CDRs 1 and 3 according to the 3E10-D31N variant. In embodiments, the 3E10 antibody or antigen-binding fragment thereof further includes VL CDRs 1-3, and VH CDRs 1 and 3 having one or more amino acid substitutions relative to the CDRs of the parent 3E10 antibody, e.g., as described herein.

In embodiments, a 3E10 antibody or antigen-binding fragment thereof includes VH CDR3 comprising the amino acid sequence of 3E10-VH-CDR3m (SEQ ID NO:60). In embodiments, the 3E10 antibody or antigen-binding fragment thereof further includes VL CDRs 1-3, and VH CDRs 1 and 2 according to the parent 3E10 antibody. In embodiments, the 3E10 antibody or antigen-binding fragment thereof further includes VL CDRs 1-3, and VH CDRs 1 and 2 according to the 3E10-D31N variant. In embodiments, the 3E10 antibody or antigen-binding fragment thereof further includes VL CDRs 1-3, and VH CDRs 1 and 2 having one or more amino acid substitutions relative to the CDRs of the parent 3E10 antibody, e.g., as described herein.

In embodiments, a 3E10 antibody or antigen-binding fragment thereof includes VL CDR1 comprising the amino acid sequence of 3E10-VL-CDR1m (SEQ ID NO:61). In embodiments, the 3E10 antibody or antigen-binding fragment thereof further includes VL CDRs 2 and 3, and VH CDRs 1-3 according to the parent 3E10 antibody. In embodiments, the 3E10 antibody or antigen-binding fragment thereof further includes VL CDRs 2 and 3, and VH CDRs 1-3 according to the 3E10-D31N variant. In embodiments, the 3E10 antibody or antigen-binding fragment thereof further includes VL CDRs 2 and 3, and VH CDRs 1-3 having one or more amino acid substitutions relative to the CDRs of the parent 3E10 antibody, e.g., as described herein.

In embodiments, a 3E10 antibody or antigen-binding fragment thereof includes VL CDR2 comprising the amino acid sequence of 3E10-VL-CDR2m (SEQ ID NO:62). In embodiments, the 3E10 antibody or antigen-binding fragment thereof further includes VL CDRs 1 and 3, and VH CDRs 1-3 according to the parent 3E10 antibody. In embodiments, the 3E10 antibody or antigen-binding fragment thereof further includes VL CDRs 1 and 3, and VH CDRs 1-3 according to the 3E10-D31N variant. In embodiments, the 3E10 antibody or antigen-binding fragment thereof further includes VL CDRs 1 and 3, and VH CDRs 1-3 having one or more amino acid substitutions relative to the CDRs of the parent 3E10 antibody, e.g., as described herein.

In embodiments, a 3E10 antibody or antigen-binding fragment thereof includes VL CDR3 comprising the amino acid sequence of 3E10-VL-CDR3m (SEQ ID NO:63). In embodiments, the 3E10 antibody or antigen-binding fragment thereof further includes VL CDRs 1 and 2, and VH CDRs 1-3 according to the parent 3E10 antibody. In embodiments, the 3E10 antibody or antigen-binding fragment thereof further includes VL CDRs 1 and 2, and VH CDRs 1-3 according to the 3E10-D31N variant. In embodiments, the 3E10 antibody or antigen-binding fragment thereof further includes VL CDRs 1 and 2, and VH CDRs 1-3 having one or more amino acid substitutions relative to the CDRs of the parent 3E10 antibody, e.g., as described herein.

In embodiments, a 3E10 antibody or antigen-binding fragment thereof described herein includes a VL CDR 1 comprising the amino acid sequence of 3E10-VL-CDR1m (SEQ ID NO: 61), a VL CDR2 comprising the amino acid sequence of 3E10-VL-CDR2m (SEQ ID NO:62), a VL CDR3 comprising the amino acid sequence of 3E10-VL-CDR3m (SEQ ID NO:63), a heavy chain variable region (VH) CDR1 comprising the amino acid sequence of 3E10-VH-CDR1m (SEQ ID NO:58), a VH CDR2 comprising the amino acid sequence of 3E10-VH-CDR2m (SEQ ID NO: 59), and a VH CDR3 comprising the amino acid sequence of 3E10-VH-CDR3m (SEQ ID NO: 60).

In embodiments, a 3E10 antibody or antigen-binding fragment thereof described herein refers to CDR sequences having no more than one amino acid substitution relative to the parent 3E10 antibody optionally including a D31N amino acid substitution in the VH CDR1. Accordingly, in embodiments, a 3E10 antibody or antigen-binding fragment thereof includes a VL CDR 1 comprising an amino acid sequence having no more than one amino acid substitution relative to 3E10-VL-CDR1 (SEQ ID NO:9), a VL CDR2 comprising an amino acid sequence having no more than one amino acid substitution relative to 3E10-VL-CDR2 (SEQ ID NO:10), a VL CDR3 comprising an amino acid sequence having no more than one amino acid substitution relative to 3E10-VL-CDR3 (SEQ ID NO:11), a heavy chain variable region (VH) CDR1 comprising an amino acid sequence having no more than one amino acid substitution relative to 3E10-VH-CDR1a (SEQ ID NO:16), a VH CDR2 comprising an amino acid sequence having no more than one amino acid substitution relative to 3E10-VH-CDR2 (SEQ ID NO:4), and a VH CDR3 comprising an amino acid sequence having no more than one amino acid substitution relative to 3E10-VH-CDR3 (SEQ ID NO:5).

In embodiments, a 3E10 antibody or antigen-binding fragment thereof described herein refers to CDR sequences having no more than two amino acid substitution relative to the parent 3E10 antibody optionally including a D31N amino acid substitution in the VH CDR1. Accordingly, in embodiments, a 3E10 antibody or antigen-binding fragment thereof includes a VL CDR 1 comprising an amino acid sequence having no more than two amino acid substitutions relative to 3E10-VL-CDR1 (SEQ ID NO:9), a VL CDR2 comprising an amino acid sequence having no more than two amino acid substitutions relative to 3E10-VL-CDR2 (SEQ ID NO:10), a VL CDR3 comprising an amino acid sequence having no more than two amino acid substitutions relative to 3E10-VL-CDR3 (SEQ ID NO:11), a heavy chain variable region (VH) CDR1 comprising an amino acid sequence having no more than two amino acid substitutions relative to 3E10-VH-CDR1a (SEQ ID NO:16), a VH CDR2 comprising an amino acid sequence having no more than two amino acid substitutions relative to 3E10-VH-CDR2 (SEQ ID NO:4), and a VH CDR3 comprising an amino acid sequence having no more than two amino acid substitutions relative to 3E10-VH-CDR3 (SEQ ID NO:5).

Other variants of a 3E10 antibody or antigen-binding fragment thereof are also known in the art, as disclosed for example, in Zack, et al., J. Immunol., 157 (5): 2082-8 (1996). For example, amino acid position 31 of the heavy chain variable region of 3E10 has been determined to be influential in the ability of the antibody and fragments thereof to penetrate nuclei and bind to DNA. A D31N mutation in CDR1 penetrates nuclei and binds DNA with much greater efficiency than the original antibody (Zack, et al., Immunology and Cell Biology, 72:513-520 (1994), Weisbart, et al., J. Autoimmun., 11, 539-546 (1998); Weisbart, Int. J. Oncol., 25, 1867-1873 (2004)). In embodiments, the antibody or antigen-binding fragment described herein has the D31N substitution.

AOCs described herein can be prepared with any 3E10 antibodies or antigen-fragments thereof, or any humanized 3E10 antibodies or antigen-fragments thereof, disclosed in the prior art. See, for example WO 2015/106290, 2016/033324, WO 2019/018426, and WO 2019/018428 (each of which is specifically incorporated by reference herein, in its entirety).

In embodiments, an AOC comprises a humanized 3E10 antibody. Generally, a humanized antibody has one or more amino acid residues introduced into it from a source that is non-human. These non-human amino acid residues are often referred to as “import” residues, which are typically taken from an “import” variable domain. Antibody humanization techniques generally involve the use of recombinant DNA technology to manipulate the DNA sequence encoding one or more polypeptide chains of an antibody molecule.

In embodiments, the disclosure provides humanized antibodies, or antigen-binding fragments thereof, that incorporate any combination of the humanized VL and VH sequences disclosed here, as well as VL and VH sequences having sequence identity thereto, e.g., having at least about 90%, at least about 91%, at least about 92%, at least about 93%, at least about 94%, at least about 95%, at least about 96%, at least about 97%, at least about 98%, or at least about 99% identity to a VH or VL sequence described herein.

In embodiments, an AOC provided herein comprises a 3E10 antibody or antigen-binding fragment thereof comprising one, two, or three CDRs of a heavy chain having the sequence of any one of SEQ ID NOs: 1, 13, or 71-84. In embodiments, an AOC provided herein comprises a 3E10 antibody or antigen-binding fragment thereof comprising one, two, or three CDRs of a heavy chain having the sequence of SEQ ID NO:1. In embodiments, an AOC provided herein comprises a 3E10 antibody or antigen-binding fragment thereof comprising one, two, or three CDRs of a heavy chain having the sequence of SEQ ID NO:13. In embodiments, an AOC provided herein comprises a 3E10 antibody or antigen-binding fragment thereof comprising one, two, or three CDRs of a heavy chain having the sequence of SEQ ID NO:71. In embodiments, an AOC provided herein comprises a 3E10 antibody or antigen-binding fragment thereof comprising one, two, or three CDRs of a heavy chain having the sequence of SEQ ID NO:72. In embodiments, an AOC provided herein comprises a 3E10 antibody or antigen-binding fragment thereof comprising one, two, or three CDRs of a heavy chain having the sequence of SEQ ID NO:73. In embodiments, an AOC provided herein comprises a 3E10 antibody or antigen-binding fragment thereof comprising one, two, or three CDRs of a heavy chain having the sequence of SEQ ID NO:74. In embodiments, an AOC provided herein comprises a 3E10 antibody or antigen-binding fragment thereof comprising one, two, or three CDRs of a heavy chain having the sequence of SEQ ID NO: 75. In embodiments, an AOC provided herein comprises a 3E10 antibody or antigen-binding fragment thereof comprising one, two, or three CDRs of a heavy chain having the sequence of SEQ ID NO:76. In embodiments, an AOC provided herein comprises a 3E10 antibody or antigen-binding fragment thereof comprising one, two, or three CDRs of a heavy chain having the sequence of SEQ ID NO:77. In embodiments, an AOC provided herein comprises a 3E10 antibody or antigen-binding fragment thereof comprising one, two, or three CDRs of a heavy chain having the sequence of SEQ ID NO: 78. In embodiments, an AOC provided herein comprises a 3E10 antibody or antigen-binding fragment thereof comprising one, two, or three CDRs of a heavy chain having the sequence of SEQ ID NO:79. In embodiments, an AOC provided herein comprises a 3E10 antibody or antigen-binding fragment thereof comprising one, two, or three CDRs of a heavy chain having the sequence of SEQ ID NO:80. In embodiments, an AOC provided herein comprises a 3E10 antibody or antigen-binding fragment thereof comprising one, two, or three CDRs of a heavy chain having the sequence of SEQ ID NO:81. In embodiments, an AOC provided herein comprises a 3E10 antibody or antigen-binding fragment thereof comprising one, two, or three CDRs of a heavy chain having the sequence of SEQ ID NO:82. In embodiments, an AOC provided herein comprises a 3E10 antibody or antigen-binding fragment thereof comprising one, two, or three CDRs of a heavy chain having the sequence of SEQ ID NO:83. In embodiments, an AOC provided herein comprises a 3E10 antibody or antigen-binding fragment thereof comprising one, two, or three CDRs of a heavy chain having the sequence of SEQ ID NO:84.

In embodiments, an AOC provided herein comprises a 3E10 antibody or antigen-binding fragment thereof comprising one, two, or three CDRs of a VH having the sequence of any one of SEQ ID NOs: 2, 14, 64-70, 103-112. In embodiments, an AOC provided herein comprises a 3E10 antibody or antigen-binding fragment thereof comprising one, two, or three CDRs of a VH having the sequence of SEQ ID NO:2. In embodiments, an AOC provided herein comprises a 3E10 antibody or antigen-binding fragment thereof comprising one, two, or three CDRs of a VH having the sequence of SEQ ID NO:14. In embodiments, an AOC provided herein comprises a 3E10 antibody or antigen-binding fragment thereof comprising one, two, or three CDRs of a VH having the sequence of SEQ ID NO:64. In embodiments, an AOC provided herein comprises a 3E10 antibody or antigen-binding fragment thereof comprising one, two, or three CDRs of a VH having the sequence of SEQ ID NO:65. In embodiments, an AOC provided herein comprises a 3E10 antibody or antigen-binding fragment thereof comprising one, two, or three CDRs of a VH having the sequence of SEQ ID NO:66. In embodiments, an AOC provided herein comprises a 3E10 antibody or antigen-binding fragment thereof comprising one, two, or three CDRs of a VH having the sequence of SEQ ID NO:67. In embodiments, an AOC provided herein comprises a 3E10 antibody or antigen-binding fragment thereof comprising one, two, or three CDRs of a VH having the sequence of SEQ ID NO:68. In embodiments, an AOC provided herein comprises a 3E10 antibody or antigen-binding fragment thereof comprising one, two, or three CDRs of a VH having the sequence of SEQ ID NO:69. In embodiments, an AOC provided herein comprises a 3E10 antibody or antigen-binding fragment thereof comprising one, two, or three CDRs of a VH having the sequence of SEQ ID NO:70. In embodiments, an AOC provided herein comprises a 3E10 antibody or antigen-binding fragment thereof comprising one, two, or three CDRs of a VH having the sequence of SEQ ID NO:103. In embodiments, an AOC provided herein comprises a 3E10 antibody or antigen-binding fragment thereof comprising one, two, or three CDRs of a VH having the sequence of SEQ ID NO:104. In embodiments, an AOC provided herein comprises a 3E10 antibody or antigen-binding fragment thereof comprising one, two, or three CDRs of a VH having the sequence of SEQ ID NO: 105. In embodiments, an AOC provided herein comprises a 3E10 antibody or antigen-binding fragment thereof comprising one, two, or three CDRs of a VH having the sequence of SEQ ID NO:106. In embodiments, an AOC provided herein comprises a 3E10 antibody or antigen-binding fragment thereof comprising one, two, or three CDRs of a VH having the sequence of SEQ ID NO:107. In embodiments, an AOC provided herein comprises a 3E10 antibody or antigen-binding fragment thereof comprising one, two, or three CDRs of a VH having the sequence of SEQ ID NO:108. In embodiments, an AOC provided herein comprises a 3E10 antibody or antigen-binding fragment thereof comprising one, two, or three CDRs of a VH having the sequence of SEQ ID NO:109. In embodiments, an AOC provided herein comprises a 3E10 antibody or antigen-binding fragment thereof comprising one, two, or three CDRs of a VH having the sequence of SEQ ID NO:110. In embodiments, an AOC provided herein comprises a 3E10 antibody or antigen-binding fragment thereof comprising one, two, or three CDRs of a VH having the sequence of SEQ ID NO:111. In embodiments, an AOC provided herein comprises a 3E10 antibody or antigen-binding fragment thereof comprising one, two, or three CDRs of a VH having the sequence of SEQ ID NO:112.

In embodiments, an AOC provided herein comprises a 3E10 antibody or antigen-binding fragment thereof comprising one, two, or three CDRs of a light chain having the sequence of any one of SEQ ID NOs: 7, 20, or 91-102. In embodiments, an AOC provided herein comprises a 3E10 antibody or antigen-binding fragment thereof comprising one, two, or three CDRs of a light chain having the sequence of SEQ ID NO:7. In embodiments, an AOC provided herein comprises a 3E10 antibody or antigen-binding fragment thereof comprising one, two, or three CDRs of a light chain having the sequence of SEQ ID NO:20. In embodiments, an AOC provided herein comprises a 3E10 antibody or antigen-binding fragment thereof comprising one, two, or three CDRs of a light chain having the sequence of SEQ ID NO:91. In embodiments, an AOC provided herein comprises a 3E10 antibody or antigen-binding fragment thereof comprising one, two, or three CDRs of a light chain having the sequence of SEQ ID NO:92. In embodiments, an AOC provided herein comprises a 3E10 antibody or antigen-binding fragment thereof comprising one, two, or three CDRs of a light chain having the sequence of SEQ ID NO:93. In embodiments, an AOC provided herein comprises a 3E10 antibody or antigen-binding fragment thereof comprising one, two, or three CDRs of a light chain having the sequence of SEQ ID NO:94. In embodiments, an AOC provided herein comprises a 3E10 antibody or antigen-binding fragment thereof comprising one, two, or three CDRs of a light chain having the sequence of SEQ ID NO:95. In embodiments, an AOC provided herein comprises a 3E10 antibody or antigen-binding fragment thereof comprising one, two, or three CDRs of a light chain having the sequence of SEQ ID NO:96. In embodiments, an AOC provided herein comprises a 3E10 antibody or antigen-binding fragment thereof comprising one, two, or three CDRs of a light chain having the sequence of SEQ ID NO:97. In embodiments, an AOC provided herein comprises a 3E10 antibody or antigen-binding fragment thereof comprising one, two, or three CDRs of a light chain having the sequence of SEQ ID NO:98. In embodiments, an AOC provided herein comprises a 3E10 antibody or antigen-binding fragment thereof comprising one, two, or three CDRs of a light chain having the sequence of SEQ ID NO:99.

In embodiments, an AOC provided herein comprises a 3E10 antibody or antigen-binding fragment thereof comprising one, two, or three CDRs of a light chain having the sequence of SEQ ID NO: 100. In embodiments, an AOC provided herein comprises a 3E10 antibody or antigen-binding fragment thereof comprising one, two, or three CDRs of a light chain having the sequence of SEQ ID NO: 101. In embodiments, an AOC provided herein comprises a 3E10 antibody or antigen-binding fragment thereof comprising one, two, or three CDRs of a light chain having the sequence of SEQ ID NO: 102.

In embodiments, an AOC provided herein comprises a 3E10 antibody or antigen-binding fragment thereof comprising one, two, or three CDRs of a VL having the sequence of any one of SEQ ID NOs: 8, 21, 85-90, or 113-121. In embodiments, an AOC provided herein comprises a 3E10 antibody or antigen-binding fragment thereof comprising one, two, or three CDRs of a VL having the sequence of SEQ ID NO:8. In embodiments, an AOC provided herein comprises a 3E10 antibody or antigen-binding fragment thereof comprising one, two, or three CDRs of a VL having the sequence of SEQ ID NO:21. In embodiments, an AOC provided herein comprises a 3E10 antibody or antigen-binding fragment thereof comprising one, two, or three CDRs of a VL having the sequence of SEQ ID NO:85. In embodiments, an AOC provided herein comprises a 3E10 antibody or antigen-binding fragment thereof comprising one, two, or three CDRs of a VL having the sequence of SEQ ID NO:86. In embodiments, an AOC provided herein comprises a 3E10 antibody or antigen-binding fragment thereof comprising one, two, or three CDRs of a VL having the sequence of SEQ ID NO:87. In embodiments, an AOC provided herein comprises a 3E10 antibody or antigen-binding fragment thereof comprising one, two, or three CDRs of a VL having the sequence of SEQ ID NO:88. In embodiments, an AOC provided herein comprises a 3E10 antibody or antigen-binding fragment thereof comprising one, two, or three CDRs of a VL having the sequence of SEQ ID NO:89. In embodiments, an AOC provided herein comprises a 3E10 antibody or antigen-binding fragment thereof comprising one, two, or three CDRs of a VL having the sequence of SEQ ID NO:90. In embodiments, an AOC provided herein comprises a 3E10 antibody or antigen-binding fragment thereof comprising one, two, or three CDRs of a VL having the sequence of SEQ ID NO:113. In embodiments, an AOC provided herein comprises a 3E10 antibody or antigen-binding fragment thereof comprising one, two, or three CDRs of a VL having the sequence of SEQ ID NO:114. In embodiments, an AOC provided herein comprises a 3E10 antibody or antigen-binding fragment thereof comprising one, two, or three CDRs of a VL having the sequence of SEQ ID NO:115. In embodiments, an AOC provided herein comprises a 3E10 antibody or antigen-binding fragment thereof comprising one, two, or three CDRs of a VL having the sequence of SEQ ID NO:116. In embodiments, an AOC provided herein comprises a 3E10 antibody or antigen-binding fragment thereof comprising one, two, or three CDRs of a VL having the sequence of SEQ ID NO:117. In embodiments, an AOC provided herein comprises a 3E10 antibody or antigen-binding fragment thereof comprising one, two, or three CDRs of a VL having the sequence of SEQ ID NO:118. In embodiments, an AOC provided herein comprises a 3E10 antibody or antigen-binding fragment thereof comprising one, two, or three CDRs of a VL having the sequence of SEQ ID NO:119. In embodiments, an AOC provided herein comprises a 3E10 antibody or antigen-binding fragment thereof comprising one, two, or three CDRs of a VL having the sequence of SEQ ID NO: 120. In embodiments, an AOC provided herein comprises a 3E10 antibody or antigen-binding fragment thereof comprising one, two, or three CDRs of a VL having the sequence of SEQ ID NO: 121.

In embodiments, an AOC provided herein comprises a 3E10 antibody or antigen-binding fragment thereof comprising one, two, three, four, five, or six CDRs of an scFv having the sequence of any one of SEQ ID NOs: 122-137. In embodiments, an AOC provided herein comprises a 3E10 antibody or antigen-binding fragment thereof comprising one, two, three, four, five, or six CDRs of an scFv having the sequence of SEQ ID NO: 122. In embodiments, an AOC provided herein comprises a 3E10 antibody or antigen-binding fragment thereof comprising one, two, three, four, five, or six CDRs of an scFv having the sequence of SEQ ID NO:123. In embodiments, an AOC provided herein comprises a 3E10 antibody or antigen-binding fragment thereof comprising one, two, three, four, five, or six CDRs of an scFv having the sequence of SEQ ID NO: 124. In embodiments, an AOC provided herein comprises a 3E10 antibody or antigen-binding fragment thereof comprising one, two, three, four, five, or six CDRs of an scFv having the sequence of SEQ ID NO:125. In embodiments, an AOC provided herein comprises a 3E10 antibody or antigen-binding fragment thereof comprising one, two, three, four, five, or six CDRs of an scFv having the sequence of SEQ ID NO:126. In embodiments, an AOC provided herein comprises a 3E10 antibody or antigen-binding fragment thereof comprising one, two, three, four, five, or six CDRs of an scFv having the sequence of SEQ ID NO: 127. In embodiments, an AOC provided herein comprises a 3E10 antibody or antigen-binding fragment thereof comprising one, two, three, four, five, or six CDRs of an scFv having the sequence of SEQ ID NO:128. In embodiments, an AOC provided herein comprises a 3E10 antibody or antigen-binding fragment thereof comprising one, two, three, four, five, or six CDRs of an scFv having the sequence of SEQ ID NO: 129. In embodiments, an AOC provided herein comprises a 3E10 antibody or antigen-binding fragment thereof comprising one, two, three, four, five, or six CDRs of an scFv having the sequence of SEQ ID NO:130. In embodiments, an AOC provided herein comprises a 3E10 antibody or antigen-binding fragment thereof comprising one, two, three, four, five, or six CDRs of an scFv having the sequence of SEQ ID NO:131. In embodiments, an AOC provided herein comprises a 3E10 antibody or antigen-binding fragment thereof comprising one, two, three, four, five, or six CDRs of an scFv having the sequence of SEQ ID NO:132. In embodiments, an AOC provided herein comprises a 3E10 antibody or antigen-binding fragment thereof comprising one, two, three, four, five, or six CDRs of an scFv having the sequence of SEQ ID NO:133. In embodiments, an AOC provided herein comprises a 3E10 antibody or antigen-binding fragment thereof comprising one, two, three, four, five, or six CDRs of an scFv having the sequence of SEQ ID NO: 134. In embodiments, an AOC provided herein comprises a 3E10 antibody or antigen-binding fragment thereof comprising one, two, three, four, five, or six CDRs of an scFv having the sequence of SEQ ID NO:135. In embodiments, an AOC provided herein comprises a 3E10 antibody or antigen-binding fragment thereof comprising one, two, three, four, five, or six CDRs of an scFv having the sequence of SEQ ID NO:136. In embodiments, an AOC provided herein comprises a 3E10 antibody or antigen-binding fragment thereof comprising one, two, three, four, five, or six CDRs of an scFv having the sequence of SEQ ID NO:137.

In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to any one of SEQ ID NOs: 1, 13, or 71-84. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:1. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:13. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO: 71. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:72. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:73. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:74. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:75. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:76. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:77. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO: 78. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:79. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:80. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:81. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:82. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:83. In embodiments, an AOC provided herein comprises a 3E10 antibody or antigen-binding fragment thereof having a heavy chain sequence having at least about 75%, at least about 80%, at least about 85%, at least about 90%, at least about 95%, at least about 96%, at least about 97%, at least about 98%, at least about 99%, or 100% identity to SEQ ID NO:84.

In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to any one of SEQ ID NOs: 7, 20, or 91-102. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:7. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:20. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:91. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:92. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:93. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:94. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO: 95. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:96. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:97. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:98. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:99. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO: 100. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO:101. In embodiments, an AOC provided herein comprises a 3E10 antibody or antigen-binding fragment thereof having a light chain sequence having at least about 75%, at least about 80%, at least about 85%, at least about 90%, at least about 95%, at least about 96%, at least about 97%, at least about 98%, at least about 99%, or 100% identity to SEQ ID NO: 102.

In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to any one of SEQ ID NOs: 2, 14, 64-70, or 103-112. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:2. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:14. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:64. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:65. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:66. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:67. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:68. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:69. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:70. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:103. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:104. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:105. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO: 106. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:107. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:108. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO: 109. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:110. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:111. In embodiments, an AOC provided herein comprises a 3E10 antibody or antigen-binding fragment thereof having a VH sequence having at least about 75%, at least about 80%, at least about 85%, at least about 90%, at least about 95%, at least about 96%, at least about 97%, at least about 98%, at least about 99%, or 100% identity to SEQ ID NO:112.

In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to any one of SEQ ID NOs: 8, 21, 85-90, or 113-121. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:8. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:21. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:85. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:86. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:87. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:88. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:89. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:90. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:113. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO: 114. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:115. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:116. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:117. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:118. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:119. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO: 120. In embodiments, an AOC provided herein comprises a 3E10 antibody or antigen-binding fragment thereof having a VL sequence having at least about 75%, at least about 80%, at least about 85%, at least about 90%, at least about 95%, at least about 96%, at least about 97%, at least about 98%, at least about 99%, or 100% identity to SEQ ID NO:121.

In embodiments, an AOC provided herein comprises a humanized 3E10 antibody, or antigen-binding fragment thereof, comprising a light chain variable domain (3E10-VL) comprising an amino acid sequence that is at least about 97% identical to an amino acid sequence selected from the group consisting of 3E10-VL-h1 (SEQ ID NO:85), 3E10-VL-h2 (SEQ ID NO: 86), 3E10-VL-h3 (SEQ ID NO:87), 3E10-VL-h4 (SEQ ID NO:88), 3E10-VL-h5 (SEQ ID NO: 89), and 3E10-VL-h6 (SEQ ID NO:90) and a heavy chain variable domain (3E10-VH) comprising an amino acid sequence that is at least about 95% identical to an amino acid sequence selected from the group consisting of 3E10-VH-h1 (SEQ ID NO:64), 3E10-VH-h2 (SEQ ID NO: 65), 3E10-VH-h3 (SEQ ID NO:66), 3E10-VH-h4 (SEQ ID NO:67), 3E10-VH-h5 (SEQ ID NO: 68), 3E10-VH-h6 (SEQ ID NO:69), and 3E10-VH-h7 (SEQ ID NO:70).

In embodiments, the sequence of the 3E10-VL is at least about 97% identical to 3E10-VL-h1 (SEQ ID NO:85). In embodiments, the sequence of the 3E10-VL is at least about 98% identical to 3E10-VL-h1 (SEQ ID NO:85). In embodiments, the sequence of the 3E10-VL is at least about 99% identical to 3E10-VL-h1 (SEQ ID NO:85). In embodiments, the sequence of the 3E10-VL is 3E10-VL-h1 (SEQ ID NO:85).

In embodiments, the sequence of the 3E10-VL is at least about 97% identical to 3E10-VL-h2 (SEQ ID NO:86). In embodiments, the sequence of the 3E10-VL is at least about 98% identical to 3E10-VL-h2 (SEQ ID NO:86). In embodiments, the sequence of the 3E10-VL is at least about 99% identical to 3E10-VL-h2 (SEQ ID NO:86). In embodiments, the sequence of the 3E10-VL is 3E10-VL-h2 (SEQ ID NO:86).

In embodiments, the sequence of the 3E10-VL is at least about 97% identical to 3E10-VL-h3 (SEQ ID NO:87). In embodiments, the sequence of the 3E10-VL is at least about 98% identical to 3E10-VL-h3 (SEQ ID NO:87). In embodiments, the sequence of the 3E10-VL is at least about 99% identical to 3E10-VL-h3 (SEQ ID NO:87). In embodiments, the sequence of the 3E10-VL is 3E10-VL-h3 (SEQ ID NO:87).

In embodiments, the sequence of the 3E10-VL is at least about 97% identical to 3E10-VL-h4 (SEQ ID NO:88). In embodiments, the sequence of the 3E10-VL is at least about 98% identical to 3E10-VL-h4 (SEQ ID NO:88). In embodiments, the sequence of the 3E10-VL is at least about 99% identical to 3E10-VL-h4 (SEQ ID NO:88). In embodiments, the sequence of the 3E10-VL is 3E10-VL-h4 (SEQ ID NO:88).

In embodiments, the sequence of the 3E10-VL is at least about 97% identical to 3E10-VL-h5 (SEQ ID NO:89). In embodiments, the sequence of the 3E10-VL is at least about 98% identical to 3E10-VL-h5 (SEQ ID NO:89). In embodiments, the sequence of the 3E10-VL is at least about 99% identical to 3E10-VL-h5 (SEQ ID NO:89). In embodiments, the sequence of the 3E10-VL is 3E10-VL-h5 (SEQ ID NO:89).

In embodiments, the sequence of the 3E10-VL is at least about 97% identical to 3E10-VL-h6 (SEQ ID NO:90). In embodiments, the sequence of the 3E10-VL is at least about 98% identical to 3E10-VL-h6 (SEQ ID NO:90). In embodiments, the sequence of the 3E10-VL is at least about 99% identical to 3E10-VL-h6 (SEQ ID NO:90). In embodiments, the sequence of the 3E10-VL is 3E10-VL-h6 (SEQ ID NO:90).

In embodiments, the sequence of the 3E10-VH is at least about 95% identical to 3E10-VH-h1 (SEQ ID NO:64). In embodiments, the sequence of the 3E10-VH is at least about 96% identical to 3E10-VH-h1 (SEQ ID NO:64). In embodiments, the sequence of the 3E10-VH is at least about 97% identical to 3E10-VH-h1 (SEQ ID NO:64). In embodiments, the sequence of the 3E10-VH is at least about 98% identical to 3E10-VH-h1 (SEQ ID NO:64). In embodiments, the sequence of the 3E10-VH is at least about 99% identical to 3E10-VH-h1 (SEQ ID NO:64). In embodiments, the sequence of the 3E10-VH is 3E10-VH-h1 (SEQ ID NO:64).

In embodiments, the sequence of the 3E10-VH is at least about 95% identical to 3E10-VH-h2 (SEQ ID NO:65). In embodiments, the sequence of the 3E10-VH is at least about 96% identical to 3E10-VH-h2 (SEQ ID NO:65). In embodiments, the sequence of the 3E10-VH is at least about 97% identical to 3E10-VH-h2 (SEQ ID NO:65). In embodiments, the sequence of the 3E10-VH is at least about 98% identical to 3E10-VH-h2 (SEQ ID NO:65). In embodiments, the sequence of the 3E10-VH is at least about 99% identical to 3E10-VH-h2 (SEQ ID NO:65). In embodiments, the sequence of the 3E10-VH is 3E10-VH-h2 (SEQ ID NO:65).

In embodiments, the sequence of the 3E10-VH is at least about 95% identical to 3E10-VH-h3 (SEQ ID NO:66). In embodiments, the sequence of the 3E10-VH is at least about 96% identical to 3E10-VH-h3 (SEQ ID NO:66). In embodiments, the sequence of the 3E10-VH is at least about 97% identical to 3E10-VH-h3 (SEQ ID NO:66). In embodiments, the sequence of the 3E10-VH is at least about 98% identical to 3E10-VH-h3 (SEQ ID NO:66). In embodiments, the sequence of the 3E10-VH is at least about 99% identical to 3E10-VH-h3 (SEQ ID NO:66). In embodiments, the sequence of the 3E10-VH is 3E10-VH-h3 (SEQ ID NO:66).

In embodiments, the sequence of the 3E10-VH is at least about 95% identical to 3E10-VH-h4 (SEQ ID NO:67). In embodiments, the sequence of the 3E10-VH is at least about 96% identical to 3E10-VH-h4 (SEQ ID NO:67). In embodiments, the sequence of the 3E10-VH is at least about 97% identical to 3E10-VH-h4 (SEQ ID NO:67). In embodiments, the sequence of the 3E10-VH is at least about 98% identical to 3E10-VH-h4 (SEQ ID NO:67). In embodiments, the sequence of the 3E10-VH is at least about 99% identical to 3E10-VH-h4 (SEQ ID NO:67). In embodiments, the sequence of the 3E10-VH is 3E10-VH-h4 (SEQ ID NO: 67).

In embodiments, the sequence of the 3E10-VH is at least about 95% identical to 3E10-VH-h5 (SEQ ID NO:68). In embodiments, the sequence of the 3E10-VH is at least about 96% identical to 3E10-VH-h5 (SEQ ID NO:68). In embodiments, the sequence of the 3E10-VH is at least about 97% identical to 3E10-VH-h5 (SEQ ID NO:68). In embodiments, the sequence of the 3E10-VH is at least about 98% identical to 3E10-VH-h5 (SEQ ID NO:68). In embodiments, the sequence of the 3E10-VH is at least about 99% identical to 3E10-VH-h5 (SEQ ID NO:68). In embodiments, the sequence of the 3E10-VH is 3E10-VH-h5 (SEQ ID NO:68).

In embodiments, the sequence of the 3E10-VH is at least about 95% identical to 3E10-VH-h6 (SEQ ID NO:69). In embodiments, the sequence of the 3E10-VH is at least about 96% identical to 3E10-VH-h6 (SEQ ID NO:69). In embodiments, the sequence of the 3E10-VH is at least about 97% identical to 3E10-VH-h6 (SEQ ID NO:69). In embodiments, the sequence of the 3E10-VH is at least about 98% identical to 3E10-VH-h6 (SEQ ID NO:69). In embodiments, the sequence of the 3E10-VH is at least about 99% identical to 3E10-VH-h6 (SEQ ID NO:69). In embodiments, the sequence of the 3E10-VH is 3E10-VH-h6 (SEQ ID NO:69).

In embodiments, the sequence of the 3E10-VH is at least about 95% identical to 3E10-VH-h7 (SEQ ID NO:70). In embodiments, the sequence of the 3E10-VH is at least about 96% identical to 3E10-VH-h7 (SEQ ID NO:70). In embodiments, the sequence of the 3E10-VH is at least about 97% identical to 3E10-VH-h7 (SEQ ID NO:70). In embodiments, the sequence of the 3E10-VH is at least about 98% identical to 3E10-VH-h7 (SEQ ID NO:70). In embodiments, the sequence of the 3E10-VH is at least about 99% identical to 3E10-VH-h7 (SEQ ID NO:70). In embodiments, the sequence of the 3E10-VH is 3E10-VH-h7 (SEQ ID NO:70).

In embodiments, an AOC comprising a humanized 3E10 antibody, or antigen-binding fragment thereof, described herein includes a light chain (3E10-LC) comprising an amino acid sequence that is at least about 95% identical to an amino acid sequence selected from the group consisting of 3E10-LC-h1m (SEQ ID NO:91), 3E10-LC-h2m (SEQ ID NO:92), 3E10-LC-h3m (SEQ ID NO:93), 3E10-LC-h4m (SEQ ID NO:94), 3E10-LC-h5m (SEQ ID NO:95), and 3E10-LC-h6m (SEQ ID NO:96) and a heavy chain (3E10-HC) comprising an amino acid sequence that is at least about 95% identical to an amino acid sequence selected from the group consisting of 3E10-HC-h1m (SEQ ID NO:71), 3E10-HC-h2m (SEQ ID NO:72), 3E10-HC-h3m (SEQ ID NO: 73), 3E10-HC-h4m (SEQ ID NO:74), 3E10-HC-h5m (SEQ ID NO:75), 3E10-HC-h6m (SEQ ID NO: 76), and 3E10-HC-h7m (SEQ ID NO:77).

In embodiments, the sequence of the 3E10-LC is at least about 95% identical to 3E10-LC-h1m (SEQ ID NO:91). In embodiments, the sequence of the 3E10-LC is at least about 96% identical to 3E10-LC-h1m (SEQ ID NO:91). In embodiments, the sequence of the 3E10-LC is at least about 97% identical to 3E10-LC-h1m (SEQ ID NO:91). In embodiments, the sequence of the 3E10-LC is at least about 98% identical to 3E10-LC-h1m (SEQ ID NO:91). In embodiments, the sequence of the 3E10-LC is at least about 99% identical to 3E10-LC-h1m (SEQ ID NO:91). In embodiments, the sequence of the 3E10-LC is 3E10-LC-h1m (SEQ ID NO:91).

In embodiments, the sequence of the 3E10-LC is at least about 95% identical to 3E10-LC-h2m (SEQ ID NO:92). In embodiments, the sequence of the 3E10-LC is at least about 96% identical to 3E10-LC-h2m (SEQ ID NO:92). In embodiments, the sequence of the 3E10-LC is at least about 97% identical to 3E10-LC-h2m (SEQ ID NO:92). In embodiments, the sequence of the 3E10-LC is at least about 98% identical to 3E10-LC-h2m (SEQ ID NO:92). In embodiments, the sequence of the 3E10-LC is at least about 99% identical to 3E10-LC-h2m (SEQ ID NO:92). In embodiments, the sequence of the 3E10-LC is 3E10-LC-h2m (SEQ ID NO:92).

In embodiments, the sequence of the 3E10-LC is at least about 95% identical to 3E10-LC-h3m (SEQ ID NO:93). In embodiments, the sequence of the 3E10-LC is at least about 96% identical to 3E10-LC-h3m (SEQ ID NO:93). In embodiments, the sequence of the 3E10-LC is at least about 97% identical to 3E10-LC-h3m (SEQ ID NO:93). In embodiments, the sequence of the 3E10-LC is at least about 98% identical to 3E10-LC-h3m (SEQ ID NO:93). In embodiments, the sequence of the 3E10-LC is at least about 99% identical to 3E10-LC-h3m (SEQ ID NO:93). In embodiments, the sequence of the 3E10-LC is 3E10-LC-h3m (SEQ ID NO:93).

In embodiments, the sequence of the 3E10-LC is at least about 95% identical to 3E10-LC-h4m (SEQ ID NO:94). In embodiments, the sequence of the 3E10-LC is at least about 96% identical to 3E10-LC-h4m (SEQ ID NO:94). In embodiments, the sequence of the 3E10-LC is at least about 97% identical to 3E10-LC-h4m (SEQ ID NO:94). In embodiments, the sequence of the 3E10-LC is at least about 98% identical to 3E10-LC-h4m (SEQ ID NO:94). In embodiments, the sequence of the 3E10-LC is at least about 99% identical to 3E10-LC-h4m (SEQ ID NO:94). In embodiments, the sequence of the 3E10-LC is 3E10-LC-h4m (SEQ ID NO:94).

In embodiments, the sequence of the 3E10-LC is at least about 95% identical to 3E10-LC-h5m (SEQ ID NO:95). In embodiments, the sequence of the 3E10-LC is at least about 96% identical to 3E10-LC-h5m (SEQ ID NO:95). In embodiments, the sequence of the 3E10-LC is at least about 97% identical to 3E10-LC-h5m (SEQ ID NO:95). In embodiments, the sequence of the 3E10-LC is at least about 98% identical to 3E10-LC-h5m (SEQ ID NO:95). In embodiments, the sequence of the 3E10-LC is at least about 99% identical to 3E10-LC-h5m (SEQ ID NO:95). In embodiments, the sequence of the 3E10-LC is 3E10-LC-h5m (SEQ ID NO:95).

In embodiments, the sequence of the 3E10-LC is at least about 95% identical to 3E10-LC-h6m (SEQ ID NO:96). In embodiments, the sequence of the 3E10-LC is at least about 96% identical to 3E10-LC-h6m (SEQ ID NO:96). In embodiments, the sequence of the 3E10-LC is at least about 97% identical to 3E10-LC-h6m (SEQ ID NO:96). In embodiments, the sequence of the 3E10-LC is at least about 98% identical to 3E10-LC-h6m (SEQ ID NO:96). In embodiments, the sequence of the 3E10-LC is at least about 99% identical to 3E10-LC-h6m (SEQ ID NO:96). In embodiments, the sequence of the 3E10-LC is 3E10-LC-h6m (SEQ ID NO:96).

In embodiments, the sequence of the 3E10-HC is at least about 95% identical to 3E10-HC-h1m (SEQ ID NO:71). In embodiments, the sequence of the 3E10-HC is at least about 96% identical to 3E10-HC-h1m (SEQ ID NO:71). In embodiments, the sequence of the 3E10-HC is at least about 97% identical to 3E10-HC-h1m (SEQ ID NO:71). In embodiments, the sequence of the 3E10-HC is at least about 98% identical to 3E10-HC-h1m (SEQ ID NO:71). In embodiments, the sequence of the 3E10-HC is at least about 99% identical to 3E10-HC-h1m (SEQ ID NO: 71). In embodiments, the sequence of the 3E10-HC is 3E10-HC-h1m (SEQ ID NO:71).

In embodiments, the sequence of the 3E10-HC is at least about 95% identical to 3E10-HC-h2m (SEQ ID NO:72). In embodiments, the sequence of the 3E10-HC is at least about 96% identical to 3E10-HC-h2m (SEQ ID NO:72). In embodiments, the sequence of the 3E10-HC is at least about 97% identical to 3E10-HC-h2m (SEQ ID NO:72). In embodiments, the sequence of the 3E10-HC is at least about 98% identical to 3E10-HC-h2m (SEQ ID NO:72). In embodiments, the sequence of the 3E10-HC is at least about 99% identical to 3E10-HC-h2m (SEQ ID NO: 72). In embodiments, the sequence of the 3E10-HC is 3E10-HC-h2m (SEQ ID NO:72).

In embodiments, the sequence of the 3E10-HC is at least about 95% identical to 3E10-HC-h3m (SEQ ID NO:73). In embodiments, the sequence of the 3E10-HC is at least about 96% identical to 3E10-HC-h3m (SEQ ID NO:73). In embodiments, the sequence of the 3E10-HC is at least about 97% identical to 3E10-HC-h3m (SEQ ID NO:73). In embodiments, the sequence of the 3E10-HC is at least about 98% identical to 3E10-HC-h3m (SEQ ID NO:73). In embodiments, the sequence of the 3E10-HC is at least about 99% identical to 3E10-HC-h3m (SEQ ID NO: 73). In embodiments, the sequence of the 3E10-HC is 3E10-HC-h3m (SEQ ID NO:73).

In embodiments, the sequence of the 3E10-HC is at least about 95% identical to 3E10-HC-h4m (SEQ ID NO:74). In embodiments, the sequence of the 3E10-HC is at least about 96% identical to 3E10-HC-h4m (SEQ ID NO:74). In embodiments, the sequence of the 3E10-HC is at least about 97% identical to 3E10-HC-h4m (SEQ ID NO:74). In embodiments, the sequence of the 3E10-HC is at least about 98% identical to 3E10-HC-h4m (SEQ ID NO:74). In embodiments, the sequence of the 3E10-HC is at least about 99% identical to 3E10-HC-h4m (SEQ ID NO: 74). In embodiments, the sequence of the 3E10-HC is 3E10-HC-h4m (SEQ ID NO:74).

In embodiments, the sequence of the 3E10-HC is at least about 95% identical to 3E10-HC-h5m (SEQ ID NO:75). In embodiments, the sequence of the 3E10-HC is at least about 96% identical to 3E10-HC-h5m (SEQ ID NO:75). In embodiments, the sequence of the 3E10-HC is at least about 97% identical to 3E10-HC-h5m (SEQ ID NO:75). In embodiments, the sequence of the 3E10-HC is at least about 98% identical to 3E10-HC-h5m (SEQ ID NO:75). In embodiments, the sequence of the 3E10-HC is at least about 99% identical to 3E10-HC-h5m (SEQ ID NO: 75). In embodiments, the sequence of the 3E10-HC is 3E10-HC-h5m (SEQ ID NO:75).

In embodiments, the sequence of the 3E10-HC is at least about 95% identical to 3E10-HC-h6m (SEQ ID NO:76). In embodiments, the sequence of the 3E10-HC is at least about 96% identical to 3E10-HC-h6m (SEQ ID NO:76). In embodiments, the sequence of the 3E10-HC is at least about 97% identical to 3E10-HC-h6m (SEQ ID NO:76). In embodiments, the sequence of the 3E10-HC is at least about 98% identical to 3E10-HC-h6m (SEQ ID NO:76). In embodiments, the sequence of the 3E10-HC is at least about 99% identical to 3E10-HC-h6m (SEQ ID NO: 76). In embodiments, the sequence of the 3E10-HC is 3E10-HC-h6m (SEQ ID NO:76).

In embodiments, the sequence of the 3E10-HC is at least about 95% identical to 3E10-HC-h7m (SEQ ID NO:77). In embodiments, the sequence of the 3E10-HC is at least about 96% identical to 3E10-HC-h7m (SEQ ID NO:77). In embodiments, the sequence of the 3E10-HC is at least about 97% identical to 3E10-HC-h7m (SEQ ID NO:77). In embodiments, the sequence of the 3E10-HC is at least about 98% identical to 3E10-HC-h7m (SEQ ID NO:77). In embodiments, the sequence of the 3E10-HC is at least about 99% identical to 3E10-HC-h7m (SEQ ID NO: 77). In embodiments, the sequence of the 3E10-HC is 3E10-HC-h7m (SEQ ID NO:77).

In embodiments, an AOC provided herein comprises a humanized 3E10 antibody, or antigen-binding fragment thereof, comprising a light chain (3E10-LC) comprising an amino acid sequence that is at least about 95% identical to an amino acid sequence selected from the group consisting of 3E10-LC-h1 (SEQ ID NO:97), 3E10-LC-h2 (SEQ ID NO:98), 3E10-LC-h3 (SEQ ID NO:99), 3E10-LC-h4 (SEQ ID NO: 100), 3E10-LC-h5 (SEQ ID NO:101), and 3E10-LC-h6 (SEQ ID NO:102) and a heavy chain (3E10-HC) comprising an amino acid sequence that is at least about 95% identical to an amino acid sequence selected from the group consisting of 3E10-HC-h1 (SEQ ID NO:78), 3E10-HC-h2 (SEQ ID NO:79), 3E10-HC-h3 (SEQ ID NO:80), 3E10-HC-h4 (SEQ ID NO:81), 3E10-HC-h5 (SEQ ID NO:82), 3E10-HC-h6 (SEQ ID NO:83), and 3E10-HC-h7 (SEQ ID NO:84).

In embodiments, the sequence of the 3E10-LC is at least about 95% identical to 3E10-LC-h1 (SEQ ID NO:97. In embodiments, the sequence of the 3E10-LC is at least about 96% identical to 3E10-LC-h1 (SEQ ID NO:97). In embodiments, the sequence of the 3E10-LC is at least about 97% identical to 3E10-LC-h1 (SEQ ID NO:97). In embodiments, the sequence of the 3E10-LC is at least about 98% identical to 3E10-LC-h1 (SEQ ID NO:97). In embodiments, the sequence of the 3E10-LC is at least about 99% identical to 3E10-LC-h1 (SEQ ID NO:97). In embodiments, the sequence of the 3E10-LC is 3E10-LC-h1 (SEQ ID NO:97).

In embodiments, the sequence of the 3E10-LC is at least about 95% identical to 3E10-LC-h2 (SEQ ID NO:98). In embodiments, the sequence of the 3E10-LC is at least about 96% identical to 3E10-LC-h2 (SEQ ID NO:98). In embodiments, the sequence of the 3E10-LC is at least about 97% identical to 3E10-LC-h2 (SEQ ID NO:98). In embodiments, the sequence of the 3E10-LC is at least about 98% identical to 3E10-LC-h2 (SEQ ID NO:98). In embodiments, the sequence of the 3E10-LC is at least about 99% identical to 3E10-LC-h2 (SEQ ID NO:98). In embodiments, the sequence of the 3E10-LC is 3E10-LC-h2 (SEQ ID NO:98).

In embodiments, the sequence of the 3E10-LC is at least about 95% identical to 3E10-LC-h3 (SEQ ID NO:99). In embodiments, the sequence of the 3E10-LC is at least about 96% identical to 3E10-LC-h3 (SEQ ID NO:99). In embodiments, the sequence of the 3E10-LC is at least about 97% identical to 3E10-LC-h3 (SEQ ID NO:99). In embodiments, the sequence of the 3E10-LC is at least about 98% identical to 3E10-LC-h3 (SEQ ID NO:99). In embodiments, the sequence of the 3E10-LC is at least about 99% identical to 3E10-LC-h3 (SEQ ID NO:99). In embodiments, the sequence of the 3E10-LC is 3E10-LC-h3 (SEQ ID NO:99).

In embodiments, the sequence of the 3E10-LC is at least about 95% identical to 3E10-LC-h4 (SEQ ID NO:100). In embodiments, the sequence of the 3E10-LC is at least about 96% identical to 3E10-LC-h4 (SEQ ID NO:100). In embodiments, the sequence of the 3E10-LC is at least about 97% identical to 3E10-LC-h4 (SEQ ID NO:100). In embodiments, the sequence of the 3E10-LC is at least about 98% identical to 3E10-LC-h4 (SEQ ID NO:100). In embodiments, the sequence of the 3E10-LC is at least about 99% identical to 3E10-LC-h4 (SEQ ID NO:100). In embodiments, the sequence of the 3E10-LC is 3E10-LC-h4 (SEQ ID NO:100).

In embodiments, the sequence of the 3E10-LC is at least about 95% identical to 3E10-LC-h5 (SEQ ID NO:101). In embodiments, the sequence of the 3E10-LC is at least about 96% identical to 3E10-LC-h5 (SEQ ID NO:101). In embodiments, the sequence of the 3E10-LC is at least about 97% identical to 3E10-LC-h5 (SEQ ID NO:101). In embodiments, the sequence of the 3E10-LC is at least about 98% identical to 3E10-LC-h5 (SEQ ID NO:101). In embodiments, the sequence of the 3E10-LC is at least about 99% identical to 3E10-LC-h5 (SEQ ID NO:101). In embodiments, the sequence of the 3E10-LC is 3E10-LC-h5 (SEQ ID NO:101).

In embodiments, the sequence of the 3E10-LC is at least about 95% identical to 3E10-LC-h6 (SEQ ID NO:102). In embodiments, the sequence of the 3E10-LC is at least about 96% identical to 3E10-LC-h6 (SEQ ID NO:102). In embodiments, the sequence of the 3E10-LC is at least about 97% identical to 3E10-LC-h6 (SEQ ID NO:102). In embodiments, the sequence of the 3E10-LC is at least about 98% identical to 3E10-LC-h6 (SEQ ID NO:102). In embodiments, the sequence of the 3E10-LC is at least about 99% identical to 3E10-LC-h6 (SEQ ID NO:102). In embodiments, the sequence of the 3E10-LC is 3E10-LC-h6 (SEQ ID NO:102).

In embodiments, the sequence of the 3E10-HC is at least about 95% identical to 3E10-HC-h1 (SEQ ID NO:78). In embodiments, the sequence of the 3E10-HC is at least about 96% identical to 3E10-HC-h1 (SEQ ID NO:78). In embodiments, the sequence of the 3E10-HC is at least about 97% identical to 3E10-HC-h1 (SEQ ID NO:78). In embodiments, the sequence of the 3E10-HC is at least about 98% identical to 3E10-HC-h1 (SEQ ID NO:78). In embodiments, the sequence of the 3E10-HC is at least about 99% identical to 3E10-HC-h1 (SEQ ID NO:78). In embodiments, the sequence of the 3E10-HC is 3E10-HC-h1 (SEQ ID NO:78).

In embodiments, the sequence of the 3E10-HC is at least about 95% identical to 3E10-HC-h2 (SEQ ID NO:79). In embodiments, the sequence of the 3E10-HC is at least about 96% identical to 3E10-HC-h2 (SEQ ID NO:79). In embodiments, the sequence of the 3E10-HC is at least about 97% identical to 3E10-HC-h2 (SEQ ID NO:79). In embodiments, the sequence of the 3E10-HC is at least about 98% identical to 3E10-HC-h2 (SEQ ID NO:79). In embodiments, the sequence of the 3E10-HC is at least about 99% identical to 3E10-HC-h2 (SEQ ID NO:79). In embodiments, the sequence of the 3E10-HC is 3E10-HC-h2 (SEQ ID NO:79).

In embodiments, the sequence of the 3E10-HC is at least about 95% identical to 3E10-HC-h3 (SEQ ID NO:80). In embodiments, the sequence of the 3E10-HC is at least about 96% identical to 3E10-HC-h3 (SEQ ID NO:80). In embodiments, the sequence of the 3E10-HC is at least about 97% identical to 3E10-HC-h3 (SEQ ID NO:80). In embodiments, the sequence of the 3E10-HC is at least about 98% identical to 3E10-HC-h3 (SEQ ID NO:80). In embodiments, the sequence of the 3E10-HC is at least about 99% identical to 3E10-HC-h3 (SEQ ID NO:80). In embodiments, the sequence of the 3E10-HC is 3E10-HC-h3 (SEQ ID NO:80).

In embodiments, the sequence of the 3E10-HC is at least about 95% identical to 3E10-HC-h4 (SEQ ID NO:81). In embodiments, the sequence of the 3E10-HC is at least about 96% identical to 3E10-HC-h4 (SEQ ID NO:81). In embodiments, the sequence of the 3E10-HC is at least about 97% identical to 3E10-HC-h4 (SEQ ID NO:81). In embodiments, the sequence of the 3E10-HC is at least about 98% identical to 3E10-HC-h4 (SEQ ID NO:81). In embodiments, the sequence of the 3E10-HC is at least about 99% identical to 3E10-HC-h4 (SEQ ID NO:81). In embodiments, the sequence of the 3E10-HC is 3E10-HC-h4 (SEQ ID NO:81).

In embodiments, the sequence of the 3E10-HC is at least about 95% identical to 3E10-HC-h5 (SEQ ID NO:82). In embodiments, the sequence of the 3E10-HC is at least about 96% identical to 3E10-HC-h5 (SEQ ID NO:82). In embodiments, the sequence of the 3E10-HC is at least about 97% identical to 3E10-HC-h5 (SEQ ID NO:82). In embodiments, the sequence of the 3E10-HC is at least about 98% identical to 3E10-HC-h5 (SEQ ID NO:82). In embodiments, the sequence of the 3E10-HC is at least about 99% identical to 3E10-HC-h5 (SEQ ID NO:82). In embodiments, the sequence of the 3E10-HC is 3E10-HC-h5 (SEQ ID NO:82).

In embodiments, the sequence of the 3E10-HC is at least about 95% identical to 3E10-HC-h6 (SEQ ID NO:83). In embodiments, the sequence of the 3E10-HC is at least about 96% identical to 3E10-HC-h6 (SEQ ID NO:83). In embodiments, the sequence of the 3E10-HC is at least about 97% identical to 3E10-HC-h6 (SEQ ID NO:83). In embodiments, the sequence of the 3E10-HC is at least about 98% identical to 3E10-HC-h6 (SEQ ID NO:83). In embodiments, the sequence of the 3E10-HC is at least about 99% identical to 3E10-HC-h6 (SEQ ID NO:83). In some aspects, the sequence of the 3E10-HC is 3E10-HC-h6 (SEQ ID NO:83).

In embodiments, the sequence of the 3E10-HC is at least about 95% identical to 3E10-HC-h7 (SEQ ID NO:84). In embodiments, the sequence of the 3E10-HC is at least about 96% identical to 3E10-HC-h7 (SEQ ID NO:84). In embodiments, the sequence of the 3E10-HC is at least about 97% identical to 3E10-HC-h7 (SEQ ID NO:84). In embodiments, the sequence of the 3E10-HC is at least about 98% identical to 3E10-HC-h7 (SEQ ID NO:84). In embodiments, the sequence of the 3E10-HC is at least about 99% identical to 3E10-HC-h7 (SEQ ID NO:84). In embodiments, the sequence of the 3E10-HC is 3E10-HC-h7 (SEQ ID NO:84).

In embodiments, an AOC comprising a humanized 3E10 antibody or antigen-binding fragment thereof described herein has CDR sequences corresponding to those in the parent 3E10 antibody, optionally including a D31N amino acid substitution in the VH CDR1. Accordingly, in embodiments, a humanized 3E10 antibody or antigen-binding fragment thereof includes a light chain variable domain (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable domain (VH) CDR1 comprising the amino acid sequence of 3E10-VH-CDR1a (SEQ ID NO: 16), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

In embodiments, an AOC comprising a humanized 3E10 antibody or antigen-binding fragment thereof described herein includes CDR sequences from a variant humanized 3E10 antibody that includes a D31N amino acid substitution in the VH CDR1 (SEQ ID NO:15).

In embodiments, an AOC comprising a humanized 3E10 antibody or antigen-binding fragment thereof described herein includes a set of complementarity determining regions (CDRs) collectively having no more than seven amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than ten amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than nine amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than eight amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than seven amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than six amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than five amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than four amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than three amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than two amino acid substitutions, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In embodiments, an AOC comprising a humanized 3E10 antibody or antigen-binding fragment thereof described herein includes a set of complementarity determining regions (CDRs) collectively having no more than one amino acid substitution, relative to the set of CDRs having the amino acid sequences of 3E10-VL-CDR1 (SEQ ID NO:9), 3E10-VL-CDR2 (SEQ ID NO: 10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

Accordingly, in embodiments, an AOC described herein can comprise a humanized 3E10 antibody or antigen-binding fragment thereof includes a light chain variable domain (VL) complementarity determining region (CDR) 1 comprising the amino acid sequence of 3E10-VL-CDR1 (SEQ ID NO: 9), a VL CDR2 comprising the amino acid sequence of 3E10-VL-CDR2 (SEQ ID NO: 10), a VL CDR3 comprising the amino acid sequence of 3E10-VL-CDR3 (SEQ ID NO: 11), a heavy chain variable domain (VH) CDR 1 comprising the amino acid sequence of 3E10-VH-CDR1_D31N (SEQ ID NO: 15), a VH CDR2 comprising the amino acid sequence of 3E10-VH-CDR2 (SEQ ID NO: 4), and a VH CDR3 comprising the amino acid sequence of 3E10-VH-CDR3 (SEQ ID NO: 5).

In embodiments, an AOC comprising a humanized 3E10 antibody or antigen-binding fragment thereof described herein includes a set of complementarity determining regions (CDRs) collectively having no more than 12, 11, 10, 9, 8, 7, 6, 5, 4, 3, 2, or 1 amino acid substitutions, relative to the CDR sequences of 3E10-D31N variant (SEQ ID NOs: 15-18 and 22-24), selected from, but not limited to, a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in embodiments, an AOC comprising a humanized 3E10 antibody or antigen-binding fragment thereof includes VH CDR2 comprising the amino acid sequence of 3E10-VH-CDR2.1 (SEQ ID NO: 26) or 3E10-VH-CDR2.2 (SEQ ID NO: 27). In embodiments, the 3E10 antibody or antigen-binding fragment thereof further includes VL CDRs 1-3 (SEQ ID NOs: 9-11), and VH CDRs 1 and 3 (SEQ ID NOs: 3 and 5) according to the parent 3E10 antibody. In embodiments, the 3E10 antibody or antigen-binding fragment thereof further includes VL CDRs 1-3 (SEQ ID NOs: 22-24), and VH CDRs 1 and 3 (SEQ ID NOs: 15 and 18) according to the 3E10-D31N variant.

Similarly, in embodiments, an AOC comprising a humanized 3E10 antibody or antigen-binding fragment thereof includes VL CDR1 comprising the amino acid sequence of 3E10-VL-CDR1.1 (SEQ ID NO: 28) or 3E10-VL-CDR1.2 (SEQ ID NO: 29). In embodiments, the 3E10 antibody or antigen-binding fragment thereof further includes VL CDRs 2 and 3 (SEQ ID NOs: 10 and 11), and VH CDRs 1-3 (SEQ ID NOs: 3-5) according to the parent 3E10 antibody. In embodiments, the humanized 3E10 antibody or antigen-binding fragment thereof further includes VL CDRs 2 and 3 (SEQ ID NOs: 23 and 24), and VH CRDs 1-3 (SEQ ID NOs: 15, 17 and 18) according to the 3E10-D31N variant.

Similarly, in embodiments, an AOC comprising a humanized 3E10 antibody or antigen-binding fragment thereof includes VL CDR2 comprising the amino acid sequence of 3E10-VL-CDR2.1 (SEQ ID NO: 30). In embodiments, the 3E10 antibody or antigen-binding fragment thereof further includes VL CDRs 1 and 3 (SEQ ID NOs: 9 and 11), and VH CDRs 1-3 (SEQ ID NOs: 3-5) according to the parent 3E10 antibody. In embodiments, the humanized 3E10 antibody or antigen-binding fragment thereof further includes VL CDRs 1 and 3 (SEQ ID NOs: 22 and 24), and VH CDRs 1-3 (SEQ ID NOs: 15, 17 and 18) according to the 3E10-D31N variant.

While some of the amino acid substitutions described above are fairly conservative substitutions—e.g., an S to T substitution at position 5 of VL CDR1-other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in embodiments, an AOC comprising a humanized 3E10 antibody or antigen-binding fragment thereof includes VH CDR2 comprising the amino acid sequence of 3E10-VH-CDR2.3 (SEQ ID NO: 31). In embodiments, the humanized 3E10 antibody or antigen-binding fragment thereof further includes VL CDRs 1-3 (SEQ ID NOs: 9-11), and VH CDRs 1 and 3 (SEQ ID NOs: 3 and 5) according to the parent 3E10 antibody. In embodiments, the 3E10 antibody or antigen-binding fragment thereof further includes VL CDRs 1-3 (SEQ ID NOs: 22-24), and VH CDRs 1 and 3 (SEQ ID NOs: 15 and 18) according to the 3E10-D31N.

Similarly, in embodiments, an AOC comprising a humanized 3E10 antibody or antigen-binding fragment thereof includes VL CDR1 comprising the amino acid sequence of 3E10-VL-CDR1.3 (SEQ ID NO: 32). In embodiments, the humanized 3E10 antibody or antigen-binding fragment thereof further includes VL CDRs 2 and 3 (SEQ ID NOs: 10 and 11), and VH CDRs 1-3 (SEQ ID NOs: 15, 17 and 18) according to the parent 3E10 antibody. In embodiments, the 3E10 antibody or antigen-binding fragment thereof further includes VL CDRs 2 and 3 (SEQ ID NOs: 23 and 24), and VH CDRs 1-3 (SEQ ID NOs: 15, 17 and 18) according to the 3E10-D31N variant.

Similarly, in embodiments, an AOC comprising a humanized 3E10 antibody or antigen-binding fragment thereof, includes VL CDR2 comprising the amino acid sequence of 3E10-VL-CDR2.2 (SEQ ID NO: 33). In embodiments, the humanized 3E10 antibody or antigen-binding fragment thereof further includes VL CDRs 1 and 3 (SEQ ID NOs: 9 and 11), and VH CDRs 1-3 (SEQ ID NOs: 3-5) according to the parent 3E10 antibody. In embodiments, the 3E10 antibody or antigen-binding fragment thereof further includes VL CDRs 1 and 3 (SEQ ID NOs: 22 and 24), and VH CDRs 1-3 (SEQ ID NOs: 15, 17 and 18) according to the 3E10-D31N variant.

Accordingly, in embodiments, an AOC comprising a humanized 3E10 antibody or antigen-binding fragment thereof includes VH CDR1 comprising the amino acid sequence of 3E10-VH-CDR1.c1 (SEQ ID NO: 34), 3E10-VH-CDR1.c2 (SEQ ID NO: 35), 3E10-VH-CDR1.c3 (SEQ ID NO: 36), 3E10-VH-CDR1.c4 (SEQ ID NO: 37), or 3E10-VH-CDR1.c5 (SEQ ID NO: 38). In embodiments, the humanized 3E10 antibody or antigen-binding fragment thereof further includes VL CDRs 1-3 (SEQ ID NOs: 9-11), and VH CDRs 2 and 3 (SEQ ID NOs: 4 and 5) according to the parent 3E10 antibody.

Similarly, in embodiments, an AOC comprising a humanized 3E10 antibody or antigen-binding fragment thereof includes VH CDR2 comprising the amino acid sequence of 3E10-VH-CDR2.c1 (SEQ ID NO: 39), 3E10-VH-CDR2.c2 (SEQ ID NO: 40), or 3E10-VH-CDR2.c3 (SEQ ID NO: 41). In embodiments, the humanized 3E10 antibody or antigen-binding fragment thereof further includes VL CDRs 1-3 (SEQ ID NOs: 9-11), and VH CDRs 1 and 3 (SEQ ID NOs: 3 and 5) according to the parent 3E10 antibody. In embodiments, the 3E10 antibody or antigen-binding fragment thereof further includes VL CDRs 1-3 (SEQ ID NOs: 22-24), and VH CDRs 1 and 3 (SEQ ID NOs: 15 and 18) according to the 3E10-D31N variant.

Similarly, in embodiments, an AOC comprising a humanized 3E10 antibody or antigen-binding fragment thereof includes VH CDR3 comprising the amino acid sequence of 3E10-VH-CDR3.c1 (SEQ ID NO: 42), 3E10-VH-CDR3.c2 (SEQ ID NO: 43), or 3E10-VH-CDR3.c3 (SEQ ID NO: 44). In embodiments, the humanized 3E10 antibody or antigen-binding fragment thereof further includes VL CDRs 1-3 (SEQ ID NOs: 9-11), and VH CDRs 1 and 2 (SEQ ID NOs: 3 and 4) according to the parent 3E10 antibody. In embodiments, the 3E10 antibody or antigen-binding fragment thereof further includes VL CDRs 1-3 (SEQ ID NOs: 22-24), and VH CDRs 1 and 2 (SEQ ID NOs: 15 and 17) according to the 3E10-D31N variant.

Similarly, in embodiments, an AOC comprising a humanized 3E10 antibody or antigen-binding fragment thereof includes VL CDR1 comprising the amino acid sequence of 3E10-VL-CDR1.c1 (SEQ ID NO: 45), 3E10-VL-CDR1.c2 (SEQ ID NO: 46), 3E10-VL-CDR1.c3 (SEQ ID NO: 47), 3E10-VL-CDR1.c4 (SEQ ID NO: 48), 3E10-VL-CDR1.c5 (SEQ ID NO: 49), or 3E10-VL-CDR1.c6 (SEQ ID NO: 50). In embodiments, the humanized 3E10 antibody or antigen-binding fragment thereof further includes VL CDRs 2 and 3 (SEQ ID NOs: 10 and 11), and VH CDRs 1-3 (SEQ ID NOs: 3-5) according to the parent 3E10 antibody. In embodiments, the humanized 3E10 antibody or antigen-binding fragment thereof further includes VL CDRs 2 and 3 (SEQ ID NOs: 23 and 24), and VH CRDs 1-3 (SEQ ID NOs: 15, 17, 18) according to the 3E10-D31N variant.

Similarly, in embodiments, an AOC comprising a humanized 3E10 antibody or antigen-binding fragment thereof includes VL CDR2 comprising the amino acid sequence of 3E10-VL-CDR2.c1 (SEQ ID NO: 51). In embodiments, the humanized 3E10 antibody or antigen-binding fragment thereof further includes VL CDRs 1 and 3 (SEQ ID NOs: 9 and 11), and VH CDRs 1-3 (SEQ ID NOs: 3-5) according to the parent 3E10 antibody. In embodiments, the humanized 3E10 antibody or antigen-binding fragment thereof further includes VL CDRs 1 and 3 (SEQ ID NOs: 22 and 24), and VH CDRs 1-3 (SEQ ID NOs: 15, 17 and 18) according to the 3E10-D31N variant.

Similarly, in some aspects, an AOC comprising a humanized 3E10 antibody or antigen-binding fragment thereof includes VL CDR3 comprising the amino acid sequence of 3E10-VL-CDR3.c1 (SEQ ID NO: 52), 3E10-VL-CDR3.c2 (SEQ ID NO: 53), 3E10-VL-CDR3.c3 (SEQ ID NO: 54), 3E10-VL-CDR3.c4 (SEQ ID NO: 55), 3E10-VL-CDR3.c5 (SEQ ID NO: 56), or 3E10-VL-CDR3.c6 (SEQ ID NO: 57). In embodiments, the humanized 3E10 antibody or antigen-binding fragment thereof further includes VL CDRs 1 and 2 (SEQ ID NOs: 9 and 10), and VH CDRs 1-3 (SEQ ID NOs: 3-5) according to the parent 3E10 antibody. In embodiments, the humanized 3E10 antibody or antigen-binding fragment thereof further includes VL CDRs 1 and 2 (SEQ ID NOs: 22 and 23), and VH CDRs 1-3 (SEQ ID NOs: 15, 17 and 18) according to the 3E10-D31N variant.

It is also contemplated that an AOC comprising a humanized 3E10 antibody or antigen-binding fragment thereof, as described herein, includes no more than 12, 11, 10, 9, 8, 7, 6, 5, 4, 3, 2, or 1 CDR amino acid substitutions of the CDR amino acid substitutions described above. Further examples of 3E10 variant CDR sequences are described herein (SEQ ID NOs: 58-63).

Accordingly, in embodiments, an AOC comprising a humanized 3E10 antibody or antigen-binding fragment thereof includes VH CDR1 comprising the amino acid sequence of 3E10-VH-CDR1m (SEQ ID NO: 58). In embodiments, the humanized 3E10 antibody or antigen-binding fragment thereof further includes VL CDRs 1-3 (SEQ ID NOs: 9-11), and VH CDRs 2 and 3 (SEQ ID NOs: 4 and 5) according to the parent 3E10 antibody.

Similarly, in embodiments, an AOC comprising a humanized 3E10 antibody or antigen-binding fragment thereof includes VH CDR2 comprising the amino acid sequence of 3E10-VH-CDR2m (SEQ ID NO: 59). In embodiments, the humanized 3E10 antibody or antigen-binding fragment thereof further includes VL CDRs 1-3 (SEQ ID NOs: 9-11), and VH CDRs 1 and 3 (SEQ ID NOs: 3 and 5) according to the parent 3E10 antibody. In embodiments, the humanized 3E10 antibody or antigen-binding fragment thereof further includes VL CDRs 1-3 (SEQ ID NOs: 22-24), and VH CDRs 1 and 3 (SEQ ID NOs: 15 and 18) according to the 3E10-D31N variant.

Similarly, in embodiments, an AOC comprising a humanized 3E10 antibody or antigen-binding fragment thereof includes VH CDR3 comprising the amino acid sequence of 3E10-VH-CDR3m (SEQ ID NO: 60). In embodiments, the humanized 3E10 antibody or antigen-binding fragment thereof further includes VL CDRs 1-3 (SEQ ID NOs: 9-11), and VH CDRs 1 and 2 (SEQ ID NOs: 3 and 4) according to the parent 3E10 antibody. In embodiments, the humanized 3E10 antibody or antigen-binding fragment thereof further includes VL CDRs 1-3 (SEQ ID NOs: 22-24), and VH CDRs 1 and 2 (SEQ ID NOs: 15 and 17) according to the 3E10-D31N variant.

Similarly, in embodiments, an AOC comprising a humanized 3E10 antibody or antigen-binding fragment thereof includes VL CDR1 comprising the amino acid sequence of 3E10-VL-CDR1m (SEQ ID NO: 61). In embodiments, the humanized 3E10 antibody or antigen-binding fragment thereof further includes VL CDRs 2 and 3 (SEQ ID NOs: 10 and 11), and VH CDRs 1-3 (SEQ ID NOs: 3-5) according to the parent 3E10 antibody. In embodiments, the humanized 3E10 antibody or antigen-binding fragment thereof further includes VL CDRs 2 and 3 (SEQ ID NOs: 23 and 24), and VH CDRs 1-3 (SEQ ID NOs: 15, 17, and 18) according to the 3E10-D31N variant.

Similarly, in embodiments, an AOC comprising a humanized 3E10 antibody or antigen-binding fragment thereof includes VL CDR2 comprising the amino acid sequence of 3E10-VL-CDR2m (SEQ ID NO: 62). In embodiments, the humanized 3E10 antibody or antigen-binding fragment thereof further includes VL CDRs 1 and 3 (SEQ ID NOs: 9 and 11), and VH CDRs 1-3 (SEQ ID NOs: 3-5) according to the parent 3E10 antibody. In embodiments, the humanized 3E10 antibody or antigen-binding fragment thereof further includes VL CDRs 1 and 3 (SEQ ID NOs: 22 and 24), and VH CDRs 1-3 (SEQ ID NOs: 15, 17 and 18) according to the 3E10-D31N variant.

Similarly, in embodiments, an AOC comprising a humanized 3E10 antibody or antigen-binding fragment thereof includes VL CDR3 comprising the amino acid sequence of 3E10-VL-CDR3m (SEQ ID NO: 63). In embodiments, the humanized 3E10 antibody or antigen-binding fragment thereof further includes VL CDRs 1 and 2 (SEQ ID NOs: 9 and 10), and VH CDRs 1-3 (SEQ ID NOs: 3-5) according to the parent 3E10 antibody. In embodiments, the humanized 3E10 antibody or antigen-binding fragment thereof further includes VL CDRs 1 and 2 (SEQ ID NOs: 9 and 10), and VH CDRs 1-3 (SEQ ID NOs: 15, 17 and 18) according to the 3E10-D31N variant.

In embodiments, an AOC comprising a humanized 3E10 antibody or antigen-binding fragment thereof described herein includes a light chain variable domain (3E10-VL) comprising an amino acid sequence that is at least about 90% identical to an amino acid sequence selected from the group consisting of 3E10-VL-h1 (SEQ ID NO:85), 3E10-VL-h2 (SEQ ID NO: 86), 3E10-VL-h3 (SEQ ID NO:87), 3E10-VL-h4 (SEQ ID NO:88), 3E10-VL-h5 (SEQ ID NO: 89), and 3E10-VL-h6 (SEQ ID NO:90), where the light chain variable domain (3E10-VL) further comprises one or more amino acid residues selected from proline (Pro) at position 15, threonine (Thr) at position 22, tyrosine (Tyr) at position 49, Thr at position 74, asparagine (Asn) at position 76, alanine (Ala) at position 80, Asn at position 81, Thr at position 83, Asn at position 85, and valine (Val) at position 104, of the 3E10-VL according to Kabat numbering, and a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), and where the antibody includes a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In embodiments, an AOC comprising the humanized 3E10 antibody or antigen-binding fragment thereof includes a set of 3E10-VL CDRs comprising no more than 5 amino acid substitutions relative to the set of CDRs having the amino acid sequences of 3E10-VL-CDR1 (SEQ ID NO: 9), 3E10-VL-CDR2 (SEQ ID NO:10), 3E10-VL-CDR3 (SEQ ID NO:11).

In embodiments, an AOC comprising the humanized 3E10 antibody or antigen-binding fragment thereof includes a set of 3E10-VL CDRs comprising no more than 4 amino acid substitutions relative to the set of CDRs having the amino acid sequences of 3E10-VL-CDR1 (SEQ ID NO: 9), 3E10-VL-CDR2 (SEQ ID NO:10), 3E10-VL-CDR3 (SEQ ID NO:11).

In embodiments, an AOC comprising the humanized 3E10 antibody or antigen-binding fragment thereof includes a set of 3E10-VL CDRs comprising no more than 3 amino acid substitutions relative to the set of CDRs having the amino acid sequences of 3E10-VL-CDR1 (SEQ ID NO: 9), 3E10-VL-CDR2 (SEQ ID NO:10), 3E10-VL-CDR3 (SEQ ID NO:11).

In embodiments, an AOC comprising the humanized 3E10 antibody or antigen-binding fragment thereof includes a set of 3E10-VL CDRs comprising no more than 2 amino acid substitutions relative to the set of CDRs having the amino acid sequences of 3E10-VL-CDR1 (SEQ ID NO: 9), 3E10-VL-CDR2 (SEQ ID NO:10), 3E10-VL-CDR3 (SEQ ID NO:11).

In embodiments, an AOC comprising the humanized 3E10 antibody or antigen-binding fragment thereof includes a set of 3E10-VL CDRs comprising no more than 1 amino acid substitution relative to the set of CDRs having the amino acid sequences of 3E10-VL-CDR1 (SEQ ID NO: 9), 3E10-VL-CDR2 (SEQ ID NO:10), 3E10-VL-CDR3 (SEQ ID NO:11).

In embodiments, an AOC comprising the humanized 3E10 antibody or antigen-binding fragment thereof includes a set of 3E10-VL CDRs comprising a set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In one aspect, the present disclosure provides an AOC comprising a humanized antibody or antigen-binding fragment thereof with a lysine (Lys) residue at position 49 of the 3E10-VL according to Kabat numbering.

In one aspect, the present disclosure provides an AOC comprising a humanized antibody or antigen-binding fragment thereof with a glutamic acid (Glu) residue at position 81 of the 3E10-VL according to Kabat numbering.

In one aspect, the present disclosure provides an AOC comprising a humanized antibody or antigen-binding fragment thereof with a proline (Pro) residue at position 15 of the 3E10-VL according to Kabat numbering.

In one aspect, the present disclosure provides an AOC comprising a humanized antibody or antigen-binding fragment thereof with a valine (Val) residue at position 104, of the 3E10-VL according to Kabat numbering.

In embodiments, an AOC comprising a humanized 3E10 antibody or antigen-binding fragment thereof described herein includes a heavy chain variable domain (3E10-VH) comprising an amino acid sequence that is at least about 90% identical to an amino acid sequence selected from the group consisting of 3E10-VH-h1 (SEQ ID NO:64), 3E10-VH-h2 (SEQ ID NO: 65), 3E10-VH-h3 (SEQ ID NO:66), 3E10-VH-h4 (SEQ ID NO:67), 3E10-VH-h5 (SEQ ID NO: 68), 3E10-VH-h6 (SEQ ID NO:69), and 3E10-VH-h7 (SEQ ID NO:70), where the heavy chain variable domain (3E10-VH) further comprises one or more amino acid residues selected from glutamine (Gln) at position 13, leucine (Leu) at position 18, arginine (Arg) at position 19, glycine (Gly) at position 42, serine (Ser) at position 49, Ser at position 77, tyrosine (Tyr) at position 79, Asn at position 82, Ala at position 84, Val at position 89, leucine (Leu) at position 108, Val at position 109, and Ser at position 113, of the 3E10-VH according to Kabat numbering, and where the antibody includes a set of 3E10-VH CDRs collectively having no more than 6 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In embodiments, an AOC comprising the humanized 3E10 antibody or antigen-binding fragment thereof includes a set of 3E10-VH CDRs comprising no more than 5 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In embodiments, an AOC comprising the humanized 3E10 antibody or antigen-binding fragment thereof includes a set of 3E10-VH CDRs comprising no more than 4 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In embodiments, an AOC comprising the humanized 3E10 antibody or antigen-binding fragment thereof includes a set of 3E10-VH CDRs comprising no more than 3 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In embodiments, an AOC comprising the humanized 3E10 antibody or antigen-binding fragment thereof includes set of 3E10-VH CDRs comprising no more than 2 amino acid substitutions relative to the a set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In embodiments, an AOC comprising the humanized 3E10 antibody or antigen-binding fragment thereof includes a set of 3E10-VH CDRs comprising no more than 1 amino acid substitution relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO: 5).

In embodiments, an AOC comprising the humanized 3E10 antibody or antigen-binding fragment thereof includes a set of 3E10-VH CDRs comprising no more than 5, 4, 3, 2, or 1 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In one aspect, the present disclosure provides an AOC comprising a humanized antibody or antigen-binding fragment thereof with an arginine (Arg) residue at position 18 of the 3E10-VH according to Kabat numbering.

In one aspect, the present disclosure provides an AOC comprising a humanized antibody or antigen-binding fragment thereof with a (Lys) residue at position 19 of the 3E10-VH according to Kabat numbering.

In one aspect, the present disclosure provides an AOC comprising a humanized antibody or antigen-binding fragment thereof with an alanine (Ala) residue at position 49 of the 3E10-VH according to Kabat numbering.

In one aspect, the present disclosure provides an AOC comprising a humanized antibody or antigen-binding fragment thereof with a glutamine (Gln) residue at position 13, of the 3E10-VH according to Kabat numbering.

In one aspect, the present disclosure provides an AOC comprising a humanized antibody or antigen-binding fragment thereof with a leucine (Leu) residue at position 108, of the 3E10-VH according to the Kabat numbering.

In one aspect, the present disclosure provides an AOC comprising a humanized antibody or antigen-binding fragment thereof with a valine (Val) residue at position 109, of the 3E10-VH according to Kabat numbering.

In one aspect, the present disclosure provides an AOC comprising a humanized antibody or antigen-binding fragment thereof with a serine (Ser) residue at position 113, of the 3E10-VH according to Kabat numbering.

In embodiments, the present disclosure provides an AOC comprising a humanized 3E10 antibody or antigen-binding fragment thereof with a fragment crystallizable (Fc) region.

In embodiments, the present disclosure provides an AOC comprising a humanized 3E10 antibody or antigen-binding fragment thereof with an Fc region selected from a human IgG1 Fc, a human IgG2a Fc, a human IgG2b Fc, a human IgG3 Fc, and a human IgG4 Fc.

In embodiments, the present disclosure provides an AOC comprising humanized 3E10 antibodies or variants thereof, or antigen-binding fragments thereof comprising a heavy chain constant domain (CH).

In embodiments, an AOC comprising the humanized 3E10 antibody or antigen-binding fragment thereof comprises an Fc region selected from a human γ1 CH1, a human γ2 CH1, a human γ3 CH1, and a human γ4 CH1.

In embodiments, the present disclosure provides an AOC comprising a humanized 3E10 antibody or antigen-binding fragment thereof comprising a light chain constant domain (CL).

In one aspect, the present disclosure provides an AOC comprising a humanized 3E10 antibody or variant comprising an Fc region selected from the group consisting of a human λ CL and a human κ CL.

In embodiments, an AOC comprising the humanized 3E10 antibody or antigen-binding fragment thereof comprising a combination of a light chain variable domain (VL) and a heavy chain variable domain (VH) selected from 3E10-VL-h1 (SEQ ID NO:85) and 3E10-VH-h1 (SEQ ID NO:64), 3E10-VL-h1 (SEQ ID NO:85) and 3E10-VH-h2 (SEQ ID NO:65), 3E10-VL-h1 (SEQ ID NO:85) and 3E10-VH-h3 (SEQ ID NO:66), 3E10-VL-h1 (SEQ ID NO:85) and 3E10-VH-h4 (SEQ ID NO:67), 3E10-VL-h2 (SEQ ID NO:86) and 3E10-VH-h1 (SEQ ID NO:64), 3E10-VL-h2 (SEQ ID NO:86) and 3E10-VH-h2 (SEQ ID NO:65), 3E10-VL-h3 (SEQ ID NO:87) and 3E10-VH-h1 (SEQ ID NO:64), 3E10-VL-h5 (SEQ ID NO:89) and 3E10-VH-h5 (SEQ ID NO:68), 3E10-VL-h5 (SEQ ID NO:89) and 3E10-VH-h6 (SEQ ID NO:69), 3E10-VL-h6 (SEQ ID NO:90) and 3E10-VH-h5 (SEQ ID NO:68), and 3E10-VL-h6 (SEQ ID NO:90) and 3E10-VH-h6 (SEQ ID NO: 69).

In embodiments, an AOC comprising the humanized 3E10 antibody or antigen-binding fragment thereof comprises a combination of a light chain variable domain (VL) of 3E10-VL-h6 (SEQ ID NO:90) and a heavy chain variable domain (VH) of 3E10-VH-h6 (SEQ ID NO: 69).

Antibodies useful in the compositions, conjugates, and methods described herein include whole immunoglobulin (i.e., an intact antibody) of any class, fragments thereof, and synthetic proteins containing at least the antigen-binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies. Therefore, the antibodies typically contain at least the CDRs necessary to maintain DNA binding.

The 3E10 antibody is typically a monoclonal 3E10, or a variant, derivative, fragment, fusion, or humanized form thereof that binds the same or different epitope(s) as 3E10.

A deposit according to the terms of the Budapest Treaty of a hybridoma cell line producing monoclonal antibody 3E10 was received on Sep. 6, 2000, and accepted by, American Type Culture Collection (ATCC), 10801 University Blvd., Manassas, VA 20110-2209, USA, and given Patent Deposit Number PTA-2439. Thus, the antibody can have the same or different epitope specificity as monoclonal antibody 3E10 produced by ATCC No. PTA 2439 hybridoma. The antibody can have the paratope of monoclonal antibody 3E10. The antibody can be a single chain variable fragment of 3E10, or a variant, e.g., a conservative variant thereof. For example, the antibody can be a single chain variable fragment of 3E10 (3E10 Fv), or a variant thereof.

Additionally, or alternatively, the heavy chain complementarity determining regions (CDRs) can be defined according to the IMGT system. The complementarity determining regions (CDRs) as identified by the IMGT system include CDR H1.3 (original sequence): GFTFSDYG (SEQ ID NO:191); CDR H1.4 (with D31N mutation): GFTFSNYG (SEQ ID NO: 192); CDR H2.2: ISSGSSTI (SEQ ID NO:193) and variant ISSSSSTI (SEQ ID NO:194); CDR H3.2: ARRGLLLDY (SEQ ID NO:195).

Additionally, or alternatively, the light chain complementarity determining regions (CDRs) can be defined according to the IMGT system. The complementarity determining regions (CDRs) as identified by the IMGT system include CDR L1.2 KSVSTSSYSY (SEQ ID NO:196) and variant KTVSTSSYSY (SEQ ID NO: 197); CDR L2.2: YAS; CDR L3.2: QHSREFPWT (SEQ ID NO: 198).

The disclosed compositions, conjugates, and methods typically utilize antibodies that maintain the ability to penetrate cells, and optionally nuclei. The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur J Immunol 38, 3178-3190 (2008), Yanase et al., J Clin Invest 100, 25-31 (1997)). The 3E10 antibodies and antigen-binding fragments thereof can transit cellular membranes via an equilibrative nucleoside (ENT) transporter. In embodiments, 3E10 transits cellular membranes via an ENT1, ENT2, ENT3 or ENT4 transporter (See, e.g., WO 2015/106290 A1 and WO 2016/033324 A1, each of which is incorporated by reference herein, in its entirety). In embodiments, 3E10 penetrates cells in an Fc-independent mechanism (as evidenced by the ability of 3E10 fragments lacking an Fc to penetrate cells) but involves presence of the nucleoside transporter ENT2 (Weisbart et al., Sci Rep 5:12022. doi: 10.1038/srep12022. (2015), Zack et al., J Immunol 157, 2082-2088 (1996), Hansen et al., J Biol Chem 282, 20790-20793 (2007)). Thus, in embodiments, the antibodies utilized in the disclosed compositions, conjugates, and methods are ones that penetrates cells in an Fc-independent mechanism and involve the presence of the nucleoside transporter ENT2.

Mutations in 3E10 that interfere with its ability to bind DNA can render the antibody incapable of nuclear penetration. Thus, typically the disclosed variants and humanized forms of the antibody maintain the ability to bind nucleic acids, particularly DNA. In addition, 3E10 scFv has previously been shown capable of penetrating into living cells and nuclei in an ENT2-dependent manner, with efficiency of uptake impaired in ENT2-deficient cells (Hansen, et al., J. Biol. Chem. 282, 20790-20793 (2007)). Thus, in embodiments, the disclosed variants and humanized forms of the antibody maintain the ability penetrate into cell nuclei in an ENT2-dependent manner.

### B. Linkers

In some aspects, the antibody-oligonucleotide conjugates (AOC) provided herein comprises a linker. The linker (L) described herein links the 3E10 antibody or antigen-binding fragment thereof to an oligonucleotide, e.g., an siRNA or antisense oligonucleotide. The term “linker” as used herein includes, without limitation, any known linker for use in antibody-oligonucleotide-conjugates known in the art. In some aspects, the AOC comprises, one, two, three, four, or more linkers.

### 1. Conjugation Sites and Methods

In some embodiments, one or more amino acids suitable conjugation of a 3E10 antibody or antigen-binding fragment thereof provided herein are selected from lysine, cysteine, histidine, arginine, aspartic acid, glutamine, serine, threonine and tyrosine. In some embodiments, one or more amino acids suitable for conjugation are introduced by substitution of one or more amino acids in the 3E10 antibody or antigen-binding fragment thereof. In some embodiments, the one or more conjugated amino acids are lysine or arginine, and conjugation is conducted via amine conjugation. In some embodiments, one or more conjugated amino acids are glutamine (Gln) and conjugation is conducted via transglutaminase (TGase) mediated enzymatic conjugation. In some embodiments, one or more conjugated amino acids are cysteine (Cys) and conjugation is conducted via thiol conjugation. For example, NHS-PEG reagent can be used to modify primary amines in a 3E10 antibody antigen-binding fragment thereof provided herein.

In particular, a method of site-specific conjugation is by means of transglutaminase. Transglutaminases (TGases) which also include bacterial transglutaminase (BTG) are a family of enzymes which catalyse the formation of a covalent bond between the γ-carbonyl-amide group of glutamines and the primary amine group of lysines.

A peptide or antibody can be a substrate for transglutaminase according to the methods of the present disclosure. Thus, in some embodiments, peptide or antibody contains a Gln or a Lys residue, and in particular a Gln residue. In some embodiments, the peptide or antibody is not a transglutaminase substrate, so one or more Gln or Lys residues, and in particular Gln residues, are inserted into the peptide or antibody sequence to make the peptide a substrate for transglutaminase. In embodiments, a Gln or Lys residue may be inserted at any position in the peptide or antibody sequence, however, it is preferably inserted at a position where the physiological properties, such as the therapeutic activity of the peptide is not affected to a degree where the peptide is not useful anymore, e.g., in a therapeutic intervention. Insertions of amino acid residues in peptides can be brought about by standard techniques known to persons skilled in the art, such as post-translational chemical modification or transgenic techniques, as described in U.S. Pat. No. 11,123,439 and US/2016/0355859, the contents of which are hereby incorporated by reference.

Since such transglutaminases also accept substrates other than lysine as amine donor, they are used to modify proteins including antibodies at suitable acceptor glutamines (Josten et al., J. Immunol. Methods 240, 47-54 (2000); Mindt et al., Bioconjugate Chem. 19, 271-278 (2008); Dennler et al., in Antibody Drug Conjugates (Ducry, L., Ed.), pp 205-215, Humana Press. (2013), the contents of which are incorporated herein by reference). Transglutaminases have been used for the conjugation of drugs to antibodies containing artificial glutamine tags which are acceptor glutamine residues which have been introduced into the antibody by genetic engineering (Strop et al., Chem. Biol. 20, 161-167 (2013)). Furthermore, the conserved glutamine residue Q295 (Kabat EU numbering) of the constant region of the heavy chain of antibodies is the only γ-carbonyl-amide donor for the bacterial transglutaminase (EC 2.3.2.13) in the backbone of aglycosylated IgG1 molecules, and is thus an acceptor glutamine, whereas no acceptor glutamine is present in the backbone of IgG1 when the antibody has been glycosylated at position N297 (Kabat EU numbering) of the heavy chain. In summary, bacterial transglutaminase can be used for the conjugation of an amine-donor substrate, for example a drug-linker construct, at an acceptor glutamine residue of an antibody. Such acceptor glutamines can be introduced by engineering of the antibody by mutations or by the generation of aglycosylated antibodies. Such aglycosylated antibodies can be introduced by deglycosylation using N-glycosidase F (PNGase F) or by mutation of N297 of the glycosylation site of the heavy chain (Kabat EU numbering) to any other amino acid except N. The enzymatic conjugation of such aglycosylated antibodies using bacterial transglutaminase has been described for aglycosylated antibody variants containing the mutations N297D, N297Q or N297S (see U.S. Pat. Nos. 9,764,038 and 9,764,038, the contents of which are incorporated by reference). The enzymatic conjugation of such aglycosylated antibodies by means of transglutaminase generally affords AOCs having a drug antibody ratio (DAR) of 2, in which both heavy chains are specifically functionalized at position Q295 (Kabat EU numbering). Only mutation N297Q of the heavy chain affords an additional conjugation site per heavy chain. The conjugation of such variants leads to AOCs having a DAR of 4, in which both heavy chains are specifically functionalized at positions Q295 and Q297.

In embodiments, the chemical modification strategy utilized to create AOCs described herein is lysine conjugation. Lysine residues in proteins, e.g., antibodies, possess a primary amine group (—NH₂) in their side chains, making them suitable targets for chemical modification. This primary amine group can react with various chemical reagents, including small molecules or polymers (e.g., cleavable and non-cleavable linkers).

In embodiments, lysine conjugation can be either site-specific or random. In site-specific conjugation, specific lysine residues within a protein, e.g., antibody, can be targeted ensuring precise control over the modification. In contrast, random conjugation involves modifying lysine residues without selectivity.

However, as described in Example 26 and FIGS. 39A-39C, tryptic peptide mapping of 3E10 conjugated at lysine residues showed that V region lysines in both VL and VH were observed to be modified and, in fact, VL K53 is the most modified lysine, occurred in nearly ⅔ of instances. Furthermore, the humanization trials described in WO 2023/168352, demonstrated that any modification to VL K53 negatively impacted the immunoreactivity to nucleic acids of the humanized 3E10-D31N monoclonal antibody (V66). This is further elucidated in Examples 27, which show greater uptake of 3E10-D31N monoclonal antibody (V66) oligonucleotide conjugates utilizing transglutaminase-mediated enzymatic conjugation in A427 cells, and in Example 28, which utilize hindered disulfide linker (SPDMV) conjugate (transglutaminase-SPDMV), and imparted the highest exon-skipping (~15%), representing a greater than 10× improvement over lysine-PEG8 AOC.

In embodiments, the conjugation strategy is selected from one of the following:

| ID No. | Conjugation strategy |
|---|---|
| CS1 | Lysine conjugation (Lys) |
| CS1A | Random lysine conjugation |
| CS1B | Site-selective lysine conjugation (Lys-azide) |
| CS2 | Transglutaminase-mediated enzymatic conjugation (Tg) |
| CS3 | Amine conjugation |
| CS4 | Thiol conjugation |
| CS5 | Ester linkage |

a. Lysine Attachment

In some embodiments of AOCs described herein, the linker moiety is conjugated to the antibody or antigen binding fragment therein through an amine linkage at one or more surface-exposed lysine residues on the antibody or antigen binding fragment thereof. Generally, lysine conjugation is a random process, with respect to which lysines are conjugated to the liker-payload. However, some preference for conjugation at certain lysines can occur due to the context of the primary, secondary, ternary, and/or quaternary structure surrounding a particular lysine residue. Many different chemistries are known in the art for attaching payloads to proteins at lysine groups. For example, activated esters on the drug-linker complexes, often O-succinimide reagents such as N-hydroxysuccinimidyl (NHS) or sulfo-NHS esters, can react with the antibody lysine residues and achieve conjugation via amide bonds, or stable amidine bonds can be generated on an antibody by the reaction of imido ester compounds, such as Traut's reagent, with antibody lysine residues. Further description of lysine conjugation techniques are described, for example, in Walker, J. M., et al., “Antibody-drug conjugates,” Humana Press (2013); Bhat, A. S., et al., “The next step in homogenous bioconjugate development: optimizing payload placement and conjugate composition,” BioProcess International (2014); and Jain, N., et al., “Current ADC linker chemistry,” Pharm Res., 32:3526-40 (2015), the disclosure of which are incorporated herein by reference, in their entireties, for all purposes.

Accordingly, in some embodiments, the present disclosure provides pharmaceutical compositions comprising an antibody-oligonucleotide conjugate (AOC) having the formula A-(L-Pᵣ)_{q}, where: A is a 3E10 antibody or antigen-binding fragment thereof as described herein, L is a linker as described herein, P is an oligonucleotide moiety as described herein, r is an integer from 1 to 4, and q is an integer from 1 to 16, in which L is conjugated to A through lysine moieties.

Advantageously, because the structural regions of antibodies contain many lysine residues, lysine conjugation can be used to generate AOC molecules with a high drug to antibody ratio (DAR). Accordingly, in some embodiments, a composition comprising an AOC where lysine attachment is used to conjugate the drug to the antibody or antigen binding fragment thereof as described herein will have an average DAR of at least 4 (an average of at least four drug moieties attached to each antibody). In some embodiments, such a composition will have an average DAR of at least 6. In some embodiments, such a composition will have an average DAR of at least 8. In some embodiments, such a composition will have an average DAR of at least 10. In some embodiments, such a composition will have an average DAR of at least 2, at least 3, at least 4, at least 5, at least 6, at least 7, at least 8, at least 9, at least 10, at least 11, at least 12, or more.

Accordingly, in some embodiments, a composition comprising an AOC where lysine attachment is used to conjugate the drug to the antibody or antigen binding fragment thereof as described herein, r is 1 and q is at least 4. In some embodiments, r is 1 and q is at least 6. In some embodiments, r is 1 and q is at least 8. In some embodiments, r is 1 and q is at least 10. In some embodiments, r is 1 and q is at least 2, at least 3, at least 4, at least 5, at least 6, at least 7, at least 8, at least 9, at least 10, at least 11, at least 12, or more. In some embodiments, a composition comprising an AOC where lysine attachment is used to conjugate the drug to the antibody or antigen binding fragment thereof as described herein, r is 2 (e.g., the linker is a branched linker) and q is at least 2. In some embodiments, r is 2 and q is at least 3. In some embodiments, r is 2 and q is at least 4. In some embodiments, r is 2 and q is at least 5. In some embodiments, r is 2 and q is at least 2, at least 3, at least 4, at least 5, at least 6, at least 7, at least 8, at least 9, at least 10, at least 11, at least 12, or more. In yet other embodiments, a composition comprising an AOC where lysine attachment is used to conjugate the drug to the antibody or antigen binding fragment thereof as described herein, the linker is highly branched, e.g., r is at least 3. In some embodiments, r is at least 4. In some embodiments, r is at least 5, 6, 7, 8, or more.

In some aspects, lysine conjugation can be either site-specific or random. In site-specific conjugation, specific lysine residues within a protein, e.g., antibody, can be targeted ensuring precise control over the modification. In contrast, random conjugation involves modifying lysine residues without selectivity.

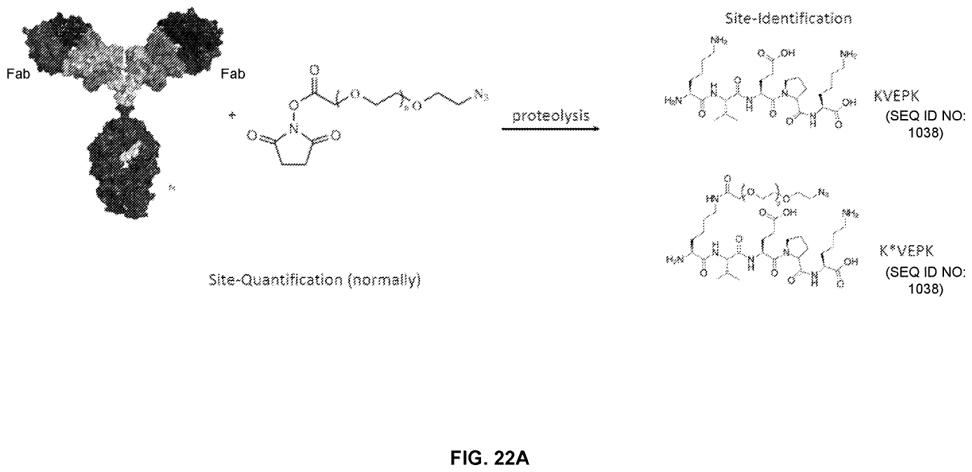

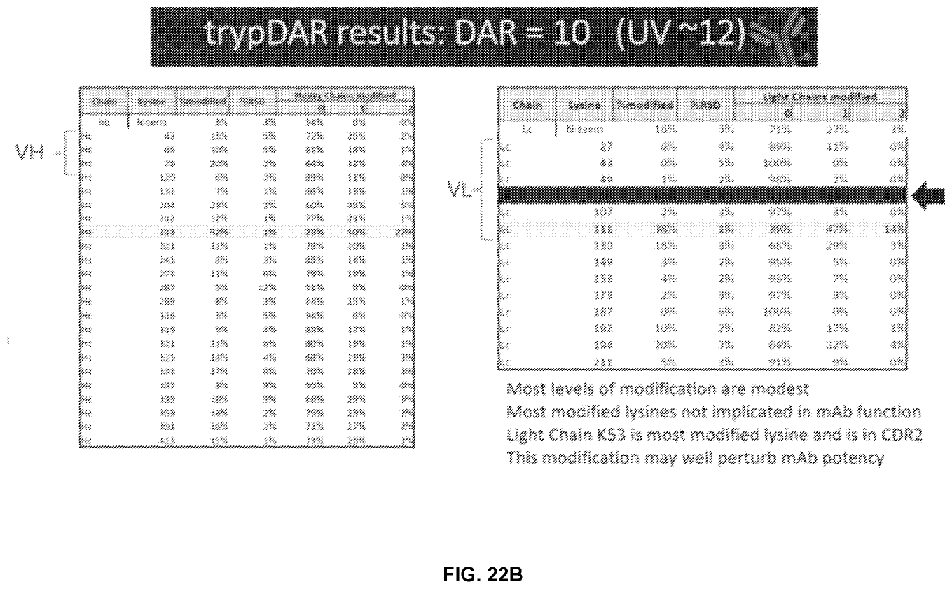

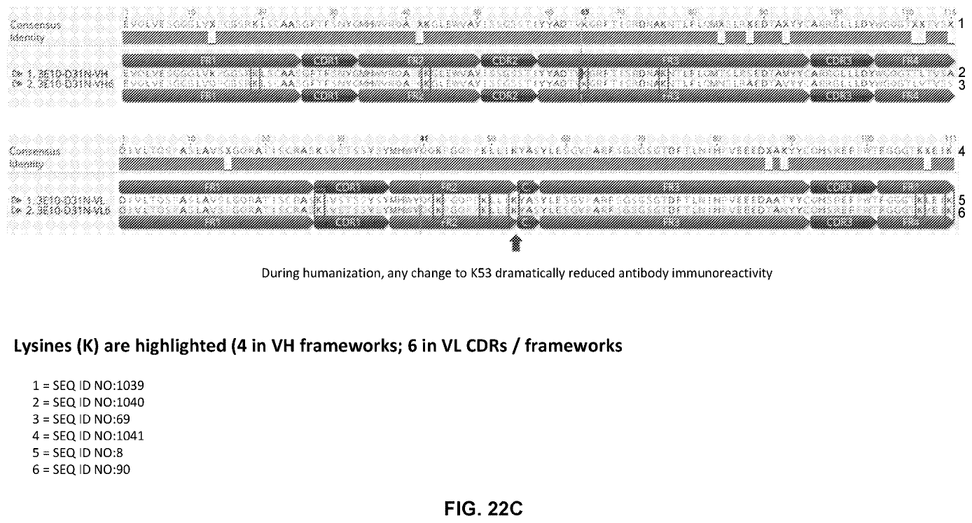

However, as described in Example 5 and FIGS. 22A-22C, tryptic peptide mapping of 3E10 conjugated at lysine residues showed that V region lysines in both VL and VH were observed to be modified and, in fact, VL K53 is the most modified lysine, occurred in nearly ⅔ of instances. Furthermore, the humanization trials described in WO 2023/168352, demonstrated that any modification to VL K53 negatively impacted the immunoreactivity to nucleic acids of the humanized 3E10-D31N monoclonal antibody (V66). This is further elucidated in Example 6, which show greater uptake of 3E10-D31N monoclonal antibody (V66) oligonucleotide conjugates utilizing transglutaminase-mediated enzymatic conjugation in A427 cells, and in Example 7, which utilize hindered disulfide linker (SPDMV) conjugate (transglutaminase-SPDMV), and imparted the highest exon-skipping (~15%), representing a greater than 10× improvement over lysine-PEG8 AOC.

b. Cysteine Attachment

In some embodiments of AOCs described herein, the linker moiety is conjugated to the antibody or antigen binding fragment therein through a sulfide linkage at one or more surface-exposed cysteine residues on the antibody or antigen binding fragment thereof. In some embodiments of AOCs described herein, the linker moiety is conjugated to the antibody or antigen binding fragment therein through a thiol side chain of one or more cysteine residues on the antibody or antigen binding fragment thereof. In general, antibodies do not possess free thiols, and all cysteine residues form disulfide bonds. In human IgG1, which is commonly used in modern ADCs, there are 4 interchain and 12 intrachain disulfide bonds. The 4 interchain disulfides, which are generally not critical for structural stability of IgG1, can be selectively reduced under mild conditions to give 2, 4, 6, or 8 free thiols while keeping the 12 intrachain disulfides intact. Due to the limited number of conjugation sites and the distinct reactivity of the thiol group, cysteine-based conjugation allows for controlled DAR and heterogeneity. Engineered Cys residues can also be used for site specific conjugation without the partial reduction of the endogenous disulfide bonds using, e.g., EnCys-mAb technology. Many different chemistries are known in the art for attaching payloads to proteins at cystine groups. For example, 8 nucleophilic cysteine residues can first be liberated from the reduced inter-chain disulfide bonds via reducing agents and later conjugated with drug-linker complexes. This approach generates ADCs with heterogeneous conjugation sites and a different number of drugs attached, resulting in a drug to antibody ratio (DAR) ranging from 0~8. Alternatively, partial reduction with either dithiothreitol (DTT) or tris(2-carboxyethyl) phosphine (TCEP) can be used to result in the disruption of the heavy-light inter-chain disulfides to release free Cys for drug conjugation, while treatment using 5,5′-dithiobis(2-nitrobenzoic acid) (DTNB) yields drug conjugates connect to the Cys residues usually involved in heavy-heavy inter-chain disulfides. Further description of cysteine conjugation techniques are described, for example, in Behrens, C. R.; et al., “Methods for site-specific drug conjugation to antibodies,” mAbs. 2014, 6 (1): 46-53; Dennler, P.; et al., “Antibody conjugates: from heterogeneous populations to defined reagents,” Antibodies. 2015, 4:197-224; and Agarwal, P.; et al., “Site-specific antibody-drug conjugates: the nexus of bioorthogonal chemistry, protein engineering, and drug development,” Bioconjugate Chem. 2015, 26:176-192, the disclosure of which are incorporated herein by reference, in their entireties, for all purposes.

Accordingly, in some embodiments, the present disclosure provides pharmaceutical compositions comprising an antibody-oligonucleotide conjugate (AOC) having the formula A-(L-Pᵣ)_{q}, where: A is a 3E10 antibody or antigen-binding fragment thereof as described herein, L is a linker as described herein, P is an oligonucleotide moiety as described herein, r is an integer from 1 to 4, and q is an integer from 1 to 16, in which L is conjugated to A through cysteine moieties.

Advantageously, because of the limited number of conjugation sites and the distinct reactivity of the thiol group, cysteine-based conjugation can be used to generate AOC molecules with a controlled DAR and heterogeneity.

Accordingly, in some embodiments, a composition comprising an AOC where cysteine attachment is used to conjugate the drug to the antibody or antigen binding fragment thereof as described herein will have an average DAR of at least 2. In some embodiments, such a composition will have an average DAR of at least 3. In some embodiments, such a composition will have an average DAR of at least 4. In some embodiments, such a composition will have an average DAR of at least 5. In some embodiments, such a composition will have an average DAR of at least 6. In some embodiments, such a composition will have an average DAR of at least 7. In some embodiments, such a composition will have an average DAR of about 8. In some embodiments, such a composition will have an average DAR of at least 2, at least 3, at least 4, at least 5, at least 6, at least 7, or about 8.

Accordingly, in some embodiments, a composition comprising an AOC where cysteine attachment is used to conjugate the drug to the antibody or antigen binding fragment thereof as described herein, r is 1 and q is at least 2. In some embodiments, r is 1 and q is at least 3. In some embodiments, r is 1 and q is at least 4. In some embodiments, r is 1 and q is at least 5. In some embodiments, r is 1 and q is at least 6. In some embodiments, r is 1 and q is at least 7. In some embodiments, r is 1 and q is about 8. In some embodiments, r is 1 and q is at least 2, at least 3, at least 4, at least 5, at least 6, at least 7, or 8. In some embodiments, a composition comprising an AOC where cysteine attachment is used to conjugate the drug to the antibody or antigen binding fragment thereof as described herein, r is 2 (e.g., the linker is a branched linker) and q is at least 2. In some embodiments, r is 2 and q is at least 3. In some embodiments, r is 2 and q is at least 4. In some embodiments, r is 2 and q is at least 5. In some embodiments, r is 2 and q is at least 6. In some embodiments, r is 2 and q is at least 7. In some embodiments, r is 2 and q is about 8. In some embodiments, r is 2 and q is at least 2, at least 3, at least 4, at least 5, at least 6, at least 7, or about 8. In yet other embodiments, a composition comprising an AOC where cysteine attachment is used to conjugate the drug to the antibody or antigen binding fragment thereof as described herein, the linker is highly branched, e.g., r is at least 3. In some embodiments, r is at least 4. In some embodiments, r is at least 5, 6, 7, 8, or more.

c. Transglutaminase-Based Attachment

In some embodiments of AOCs described herein, the linker moiety is conjugated to the antibody or antigen binding fragment therein through the primary amide side chain of one or more glutamine residues on the antibody or antigen binding fragment thereof. Generally, transglutaminase derived from Streptomyces mobaraensis catalyzes transpeptidation where a primary amine-containing linker is covalently attached to the primary amide side chain of a specific glutamine (Q295) within deglycosylated antibodies, resulting in ADCs with a defined DAR of 2 (one conjugation site per heavy chain) (Jeger et al., “Site-specific and stoichiometric modification of antibodies by bacterial transglutaminase,” Angew Chem Int Ed Engl 2010 49:9995-9997; and Dennler et al., “Transglutaminase-based chemo-enzymatic conjugation approach yields homogeneous antibody-drug conjugates,” Bioconjugate Chem 2014 25:569-578 the disclosure of which are incorporated herein by reference, in their entireties, for all purposes). An N297Q mutation prior to this conjugation provides two more reaction sites (DAR=4). This method is quite advantageous in terms of practical ADC production as the glycosidase and transglutaminase directly modify and conjugate native mAbs with the payload, without the need for genetic engineering. An alternative version using a peptide sequence-specific transglutaminase can also be used (Strop et al., “Location matters: site of conjugation modulates stability and pharmacokinetics of antibody drug conjugates,” Chem Biol 2013 20:161-167). This enzyme recognizes and utilizes LLQG (SEQ ID NO:1037) motif that is genetically incorporated, resulting in site-specific antibody-drug conjugation.

Accordingly, in some embodiments, the present disclosure provides pharmaceutical compositions comprising an antibody-oligonucleotide conjugate (AOC) having the formula A-(L-Pᵣ)_{q}, where: A is a 3E10 antibody or antigen-binding fragment thereof as described herein, L is a linker as described herein, P is a payload moiety as described herein, r is an integer from 1 to 4, and q is an integer from 1 to 16, in which L is conjugated to A through glutamine moieties.

Accordingly, in some embodiments, a composition comprising an AOC where glutamine attachment is used to conjugate the drug to the antibody or antigen binding fragment thereof as described herein will have an average Drug Antibody Ratio (DAR) of about 2. In some embodiments, such a composition will have an average DAR of at least 2. In some embodiments, such a composition will have an average DAR of at least 3. In some embodiments, such a composition will have an average DAR of about 4. In some embodiments, such a composition will have an average DAR of about 2, at least 2, at least 3, or about 4.

Accordingly, in some embodiments, a composition comprising an AOC where glutamine attachment is used to conjugate the drug to the antibody or antigen binding fragment thereof as described herein, r is 1 and q is about 2. In some embodiments, r is 1 and q is at least 2. In some embodiments, r is 1 and q is at least 3. In some embodiments, r is 1 and q is about 4. In some embodiments, r is 1 and q is about 2, at least 2, at least 3, or about 4. In some embodiments, a composition comprising an AOC where glutamine attachment is used to conjugate the drug to the antibody or antigen binding fragment thereof as described herein, r is 2 (e.g., the linker is a branched linker) and q is about 2. In some embodiments, r is 2 and q is at least 2. In some embodiments, r is 2 and q is at least 3. In some embodiments, r is 2 and q is about 4. In some embodiments, r is 2 and q is about 2, at least 2, at least 3, or about 4. In yet other embodiments, a composition comprising an AOC where cysteine attachment is used to conjugate the drug to the antibody or antigen binding fragment thereof as described herein, the linker is highly branched, e.g., r is at least 3. In some embodiments, r is at least 4. In some embodiments, r is at least 5, 6, 7, 8, or more.

### 2. Cleavable Linkers

In some aspects, the linker is a cleavable linker. As used herein, “cleavable linker” refers to a linker which can connect two or more molecules and then be cleaved once exposed to an agent. Cleavable linkers can include chemically or enzymatically unstable or degradable linkages. Cleavable linkers generally rely on processes inside the cell to liberate the drug, such as reduction in the cytoplasm, exposure to acidic conditions in the lysosome, or cleavage by specific proteases or other enzymes within the cell. Cleavable linkers generally incorporate one or more chemical bonds that are either chemically or enzymatically cleavable while the remainder of the linker is non-cleavable. In some aspects, the cleavable linker is an acid-labile linker, a phosphatase linker, glucuronidase linker, a cathepsin-B cleavable linker, a cathepsin-L cleavable linker, a protease-sensitive linker, a photo-labile linker, or a disulfide (SPDMV) transglutaminase-containing linker. In some aspects, the cleavable linker is selected from the group consisting of succinyl, O-succinyl, 4-succinimidyl-oxycarbonyl-a-(2-pyridyldithio) toluene, sulfosuccinimidyl 6-(3′-(2-pyridyldithio) propionamido) hexanoate, N-succinimidyl-3-(−2-pyridyldithio)-proprionate, succinimidyl 6-(3 (2-pyridyldithio) propionamido) hexanoate, 3-(2-pyridyldithio)-propionyl hydrazide, S-(2-thiopyridyl)-L-cysteine, N-succinimidyl 4-(2-pyridyldithio) butanoate (SPDB) (CAS: 115088-06-7), N-succinimidyl 4-(2-pyridyldithio)-2-sulfobutanoate (sulfo-SPDB), N-succinimidyl 4-(2-pyridyldithio) pentanoate (SPP) (CAS: 341498-08-6), CL2A, maleimidocaproyl-valine-citrulline-p-aminobenzoyloxycarbonyl (MC-vc-PAB), and N-succinimidyl 4-(2-pyridyldithio)-2-sulfopentanoate (sulfo-SPP). In some aspects, the cleavable linker is a homo-bi-functional linker, optionally comprising an alkyl or polyethylene glycol (PEG) chain. In some embodiments, the linker length of the polyethylene glycol (PEG) chain is 4 PEG molecules, i.e., PEG4. In some embodiments, the PEG chain is PEG8. In some other embodiments, the PEG chain is PEG12. In some aspects, the cleavable homo-bi-functional linker is selected from the group consisting of DSP (Lomant's Reagent) (CAS: 57757-57-0) and Acid-PEG4-S-S-PEG4-Acid (CAS: 2055015-40-0).

Additional examples of linkers which contain cleavable disulfide bonds include, but are not limited to “DPDPB”, 1,4-di-[3′-(2′-pyridyldithio) propionamido]butane; “SADP”, (N-succinimidyl (4-azidophenyl) 1,3′-dithio propionate); “Sulfo-SADP” (Sulfosuccinimidyl (4-azi-dophenyldithio) propionate; “DSP”-Dithio bis(succinimidylproprionate); “DTSSP”-3,3′-Dithio bis(sulfosuccinimidylpropionate); “DTBP”-dimethyl 3,3dithiobispropionimidate-2HCI. Examples of linkers cleavable by oxidation include “DST’-disuccinimidyl tartarate; and “Sulfa-DST”-disuccin imidyl tartarate.

In some embodiments, the linker L is selected from:

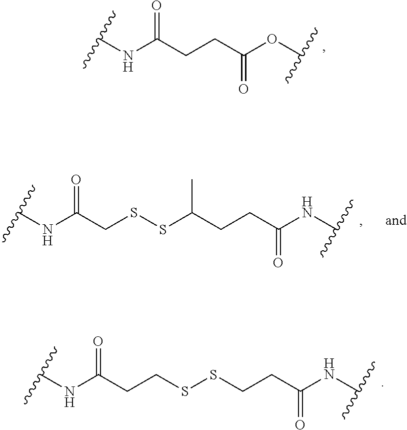 [SMILES: *NC(=O)CCC(=O)O* ; *NC(=O)CCC(C)SSCC(=O)N* ; *NC(=O)CCSSCCC(=O)N*]

In some embodiments, the linker L is selected from:

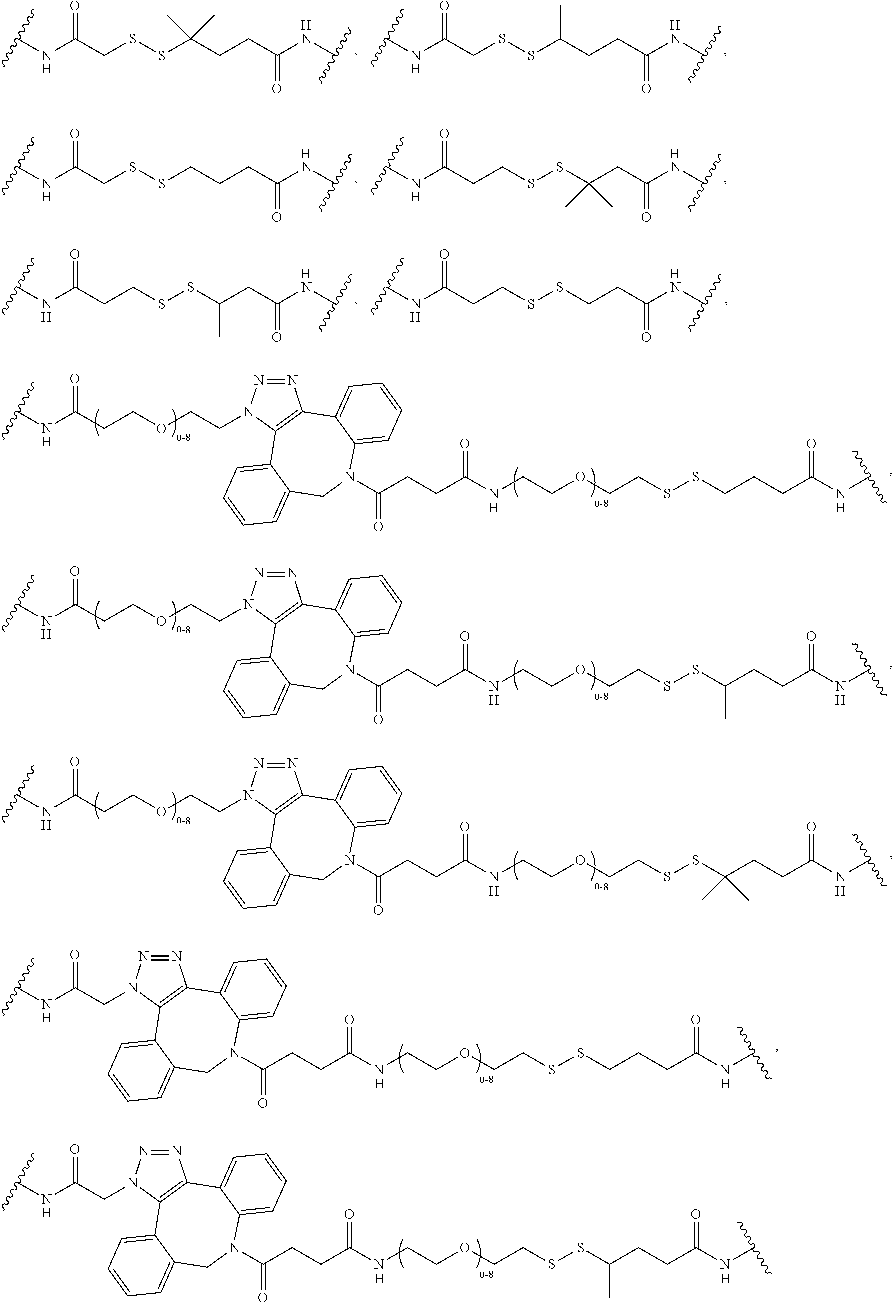

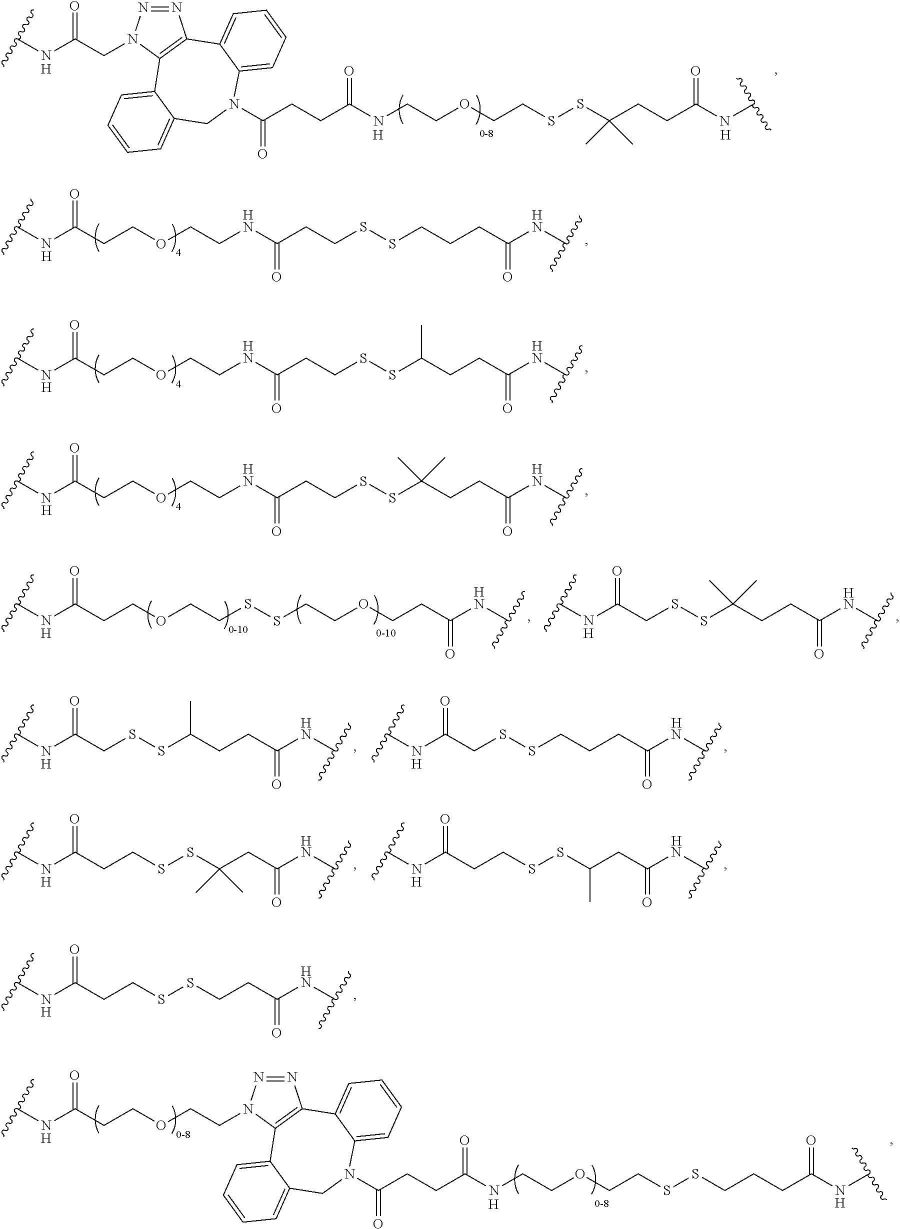

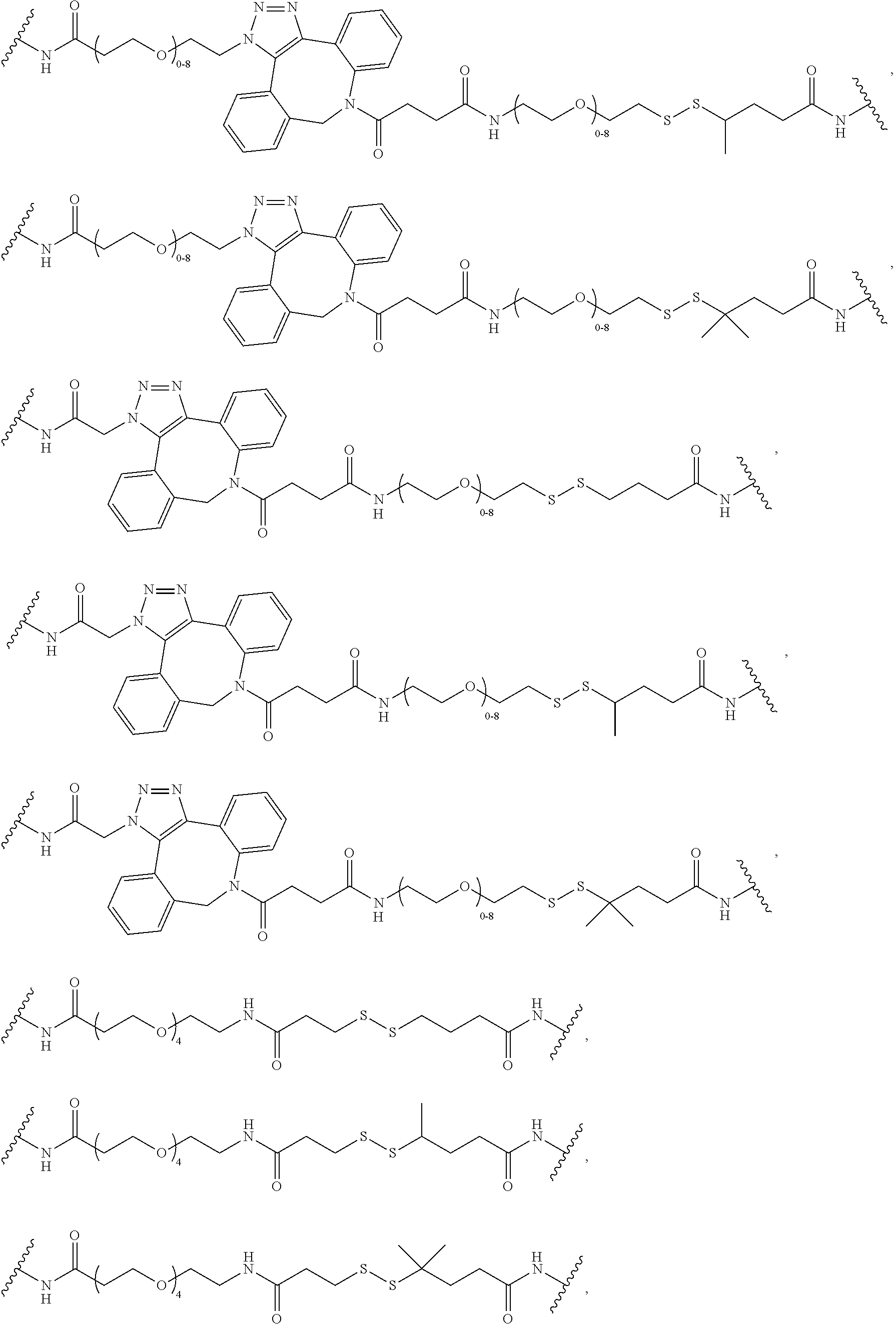

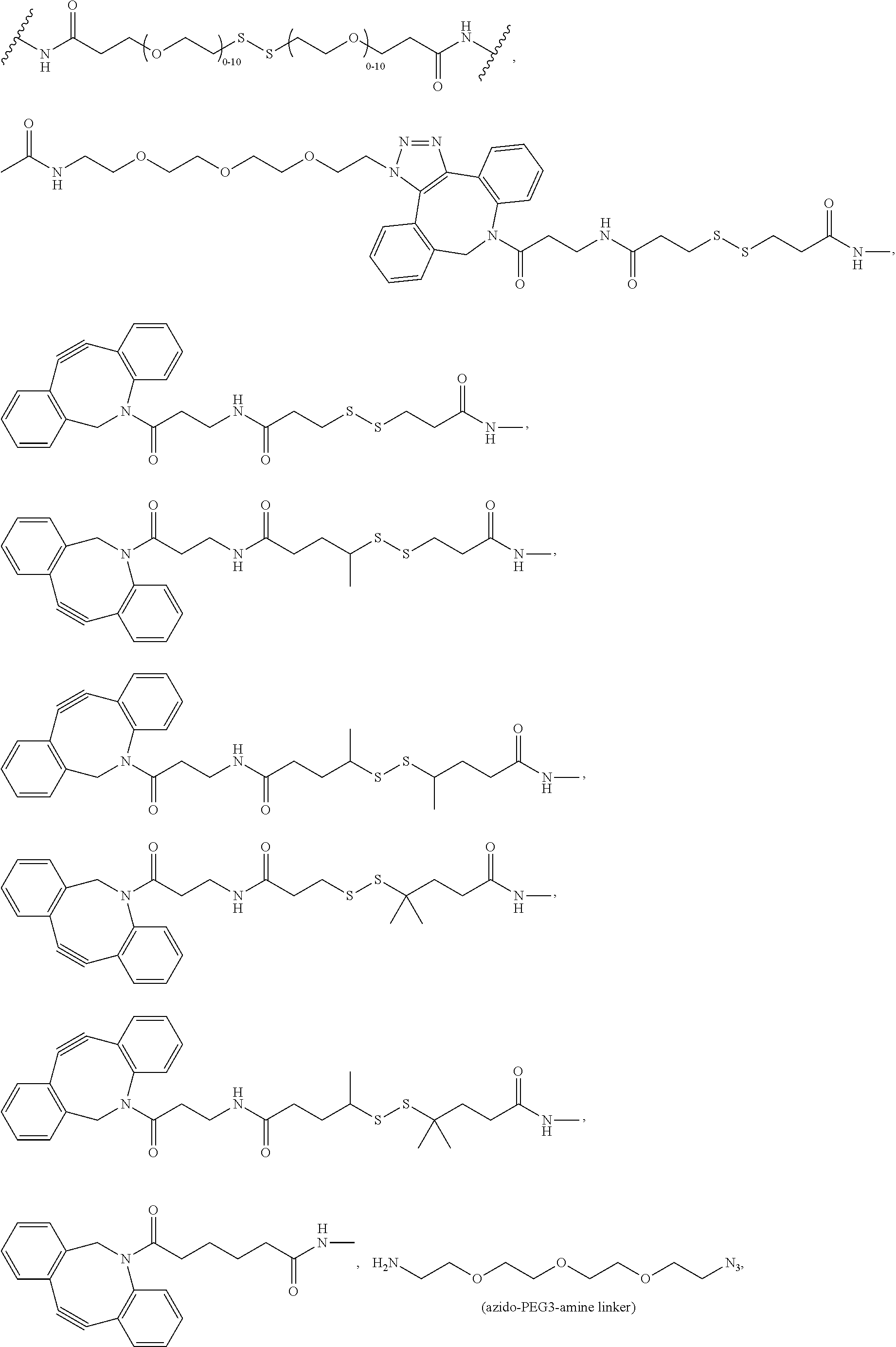

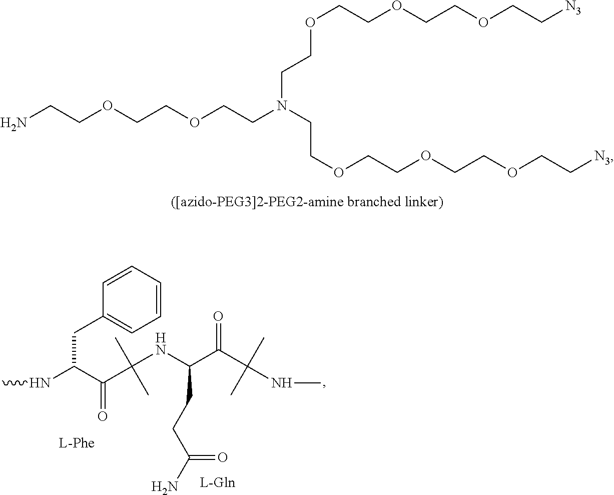

and

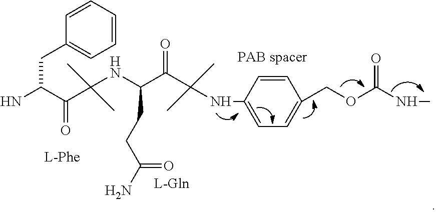

a. Cathepsin-Cleavable Linkers

In some embodiments of AOCs described herein, the linker moiety is conjugated to the antibody or antigen binding fragment therein through a cathepsin-cleavable linker.

AOCs can enter cells via receptor-mediated endocytosis, during intracellular transit or trafficking, and thus they may ultimately encounter the acidic degradative environment of the lysosome which contains multiple proteases and other catalytic enzymes for breakdown of internalized biologic substances. Most often, cleavable linkers therefore leverage an attribute of the lysosome for payload release such as lability to low pH environment, disulfide reducing environment, or cleavage by a lysosomal protease such as cathepsin B. Protease cleavable linkers or (peptide linkers) typically contain a dipeptide sequence based on deduced canonical cleavage specificity for a given protease. Common dipeptide sequences are Valine-Citrulline or Valine-Alanine. These linkers have been used frequently for conjugation of both ADCs and AOCs.

Upon release via linker cleavage, the payloads then require escape from the endosome to traffic to the specific subcellular region in order to mediate an effect.

3E10 and its derivatives, including the humanized V66 IgG1κ, are anti-DNA antibodies with the unique property of direct cell entry and subsequent trafficking to the nucleus of cells. This occurs via interaction of antibody: DNA complexes with the cell surface transporter ENT2 (equilibrative nucleoside transporter-2) and so does not require the classical endocytic mechanism employing clathrin or dynamin mediated internalization. Further, upon internalization, the antibody does not encounter early or late endosomes and, importantly, does not encounter the harsh environment of the lysosome. As a result, trafficking occurs directly to the nucleus. As such, 3E10 and its derivatives can serve as delivery vehicles for therapeutic payloads that are mechanistically active within the nucleus of target cells.

Unlike conventional antibody-drug conjugates, it is believed that 3E10 is not transported into the cell through endocytosis and, as such, 3E10 AOCs may not be exposed to the low endosomal pH conditions relied upon in some antibody-drug conjugates for cleavage of pH-sensitive linkers. As such, other strategies are needed for releasing conjugated oligonucleotides from 3E10. In embodiments, an AOC described herein comprises a cleavable linker for which the catalytic agent is present within the within the nucleus for selective release within that organelle.

It has been found that the cysteine protease Cathepsin L can also be present within the nucleus of cells. See, e.g., Goulet et al., “Increased expression and activity of nuclear cathepsin L in cancer cells suggests a novel mechanism of cell transformation,” Mol Cancer Res. 2007 September; 5(9): 899-907, incorporated herein by reference in its entirety. This ubiquitous protease has previously been shown to be present primarily in lysosomes as well as in secreted form. Translation initiation within the cathepsin L mRNA for both mouse and human cells has been shown to take place at alternative internal start sites resulting in a polypeptide lacking an NH₂-terminal signal peptide. Other cathepsins typically identified in lysosomes have also been identified in the nucleus, including cathepsin D, cathepsin V, and cathepsin B.

A nuclear form of the cysteine protease cathepsin-L is found in mouse tumor cells (and also in human tumor cells) (Goulet et al., “Increased expression and activity of nuclear cathepsin L in cancer cells suggests a novel mechanism of cell transformation,” Mol Cancer Res. 2007 September; 5(9): 899-907; and Soond et al., “Lost or Forgotten: The nuclear cathepsin protein isoforms in cancer,” Cancer Lett. 2019 Oct. 10; 462:43-50, each incorporated herein by reference in their entireties. Indeed there have been identified several proteases identified that localize to the nucleus but cathepsin-L is most prominent. Further, in cancer cells cathepsin-L appears to play a significant role in cell cycle progression and importantly tumor progression and metastasis.

In some embodiments, AOCs disclosed herein comprise dipeptides that are substrates for proteolytic cleavage by nuclear localized cathepsins, optionally cathepsin L, cathepsin S, cathepsin D, cathepsin V, and/or cathepsin B.

In some embodiments, the present disclosure provides an antibody-oligonucleotide conjugate (AOC) having the formula A-(L-Pᵣ)_{q}, where: A is a 3E10 antibody or antigen-binding fragment thereof as described herein (e.g., as described in the section titled 3E10 Antibodies and Antigen-Binding Fragments), L is a cathepsin-cleavable linker, P is a payload moiety, r is an integer from 1 to 4 and q is an integer from 1 to 16. In some embodiments, L is a cathepsin L-cleavable linker. In some embodiments, L is a cathepsin S-cleavable linker. In some embodiments, L is a cathepsin D-cleavable linker. In some embodiments, L is a cathepsin B-cleavable linker. In some embodiments, L is a cathepsin V-cleavable linker.

In embodiments, L comprises a dipeptide selected from Phe-Gln, Val-Gln, Leu-Gln, Tyr-Met, Phe-Arg, Phe-Gly, Trp-Thr, Tyr-Gly, Phe-Thr, Val-Gly, Val-Cit, Val-Arg, Thr-Thr, and Val-Thr. In embodiments, L further comprises one or more PEG molecules.

### 3. Non-Cleavable Linkers

In some aspects, the linker is a non-cleavable linker. As used herein, “non-cleavable linker” refers to linkers where the release of the biologically active molecule does not depend on, for example, the differential properties between the plasma and some cytoplasmic compartments, or whether the linker has a physical property that permits enzymatic cleavage or chemical cleavage. Non-cleavable linkers can be alkylene chains, or can be polymeric in nature, such as, for example, those based upon polyalkylene glycol polymers, amide polymers, or can include segments of alkylene chains, polyalkylene glycols and/or amide polymers. In some aspects, the non-cleavable linker is selected from DBCO-C6-NHS ester (CAS: 1384870-47-6) and DBCO-PEG8-NHS ester. In some aspects, the non-cleavable linker is a homo-bi-functional linker, optionally comprising an alkyl or polyethylene glycol (PEG) chain. In some aspects, the non-cleavable homo-bi-functional linker is selected from DSS (CAS: 68528-80-3) and Bis-PEG8-NHS ester. In embodiments, non-cleavable linkers can include N-succinimidyl (4-iodoacetyl)-aminobenzoate, sulfosuccinimidyl(4-iodoacetyl)-aminobenzoate, dichlorotriazinic acid, N-succinimidyl-[(N-maleimidopropionamido)-tetraethyleneglycol]ester (NHS-PEG4-maleimide), N-succinimidyl 4-(maleimidomethyl)cyclohexanecarboxylate (SMCC), or N-sulfosuccinimidyl 4-(maleimidomethyl)cyclohexanecarboxylate (sulfoSMCC).

Additional examples of non-cleavable linkers are “Sulfo-LC-SMPT”-(sulfosuccinimidyl 6-[alphamethyl-alpha-(2-pyridylthio) toluamido}hexanoate; “SMPT”-4-succinimidyloxycarbonyl-alpha-methyl-α(2-pyridyldithio)toluene; “ABH”-Azidobenzoyl hydrazide; “NHS-ASA”-N-Hydroxysuccinimidyl-4-azi dosalicyclic acid; “SASD”-Sulfosuccinimidyl 2-(pazidosali-cylamido)ethyl-1,3-dithiopropionate; “APDP”-N-{4-(p-azi dosalicylamido) buthy}-3′ (2′-pyidyldithio) propionamide; “BASED”-Bis-[-(4-azidosalicylamido)ethyl]disulfide; “HSAB”-N-hydroxysuccinimidyl-4 azidobenzoate; “APG”-p-Azidophenyl glyoxal monohydrate; “SANPAH”-N-Suc-ciminidyl-6 (4′-azido-2′-mitrophenyl-amimo) hexanoate; “Sulfo-SANPAH”-Sulfosuccinimidyl6-(4′-azido-2′-nitro phenylamino) hexanoate; “ANB-NOS” N-5-Azido-2-ni trobenzyoyloxysuccinimide; “SAND”-Sulfosuccinimidyl-2-(m-azido-o-mitrobenzamido)-ethyl-1,3′-dithiopr-opionate; “PNP-DTP”-p-nitrophenyl-2-diazo-3,3,3-trifluoropropi-onate; “SMCC”-Succinimidyl-4-(N-maleimidomethyl) cy clohexane-1-carboxylate; “Sulfo-SMCC”-Sulfosuccinim idyl-4-(N-maleimidomethyl)cyclohexane-1-carboxy-late; “MBS” m-Maleimidobenzoyl-N-hydroxysuccinimide ester; “sulfo-MBS”-m-Maleimidobenzoyl-N-hydroxysulfosuccin imide ester; “SIAB”-N-Succinimidyl (4-iodoacetyl) ami nobenzoate; “SulfSIAB”-N-Sulfosuccinimidyl (4-io doacetyl)aminobenzoate; “SMPB”-Succinimidyl 4-(pmalenimidophenyl) butyrate; “Sulfo-SMPB”-Sulfosuc-cinimidyl 4-(p-malenimidophenyl) butyrate; “DSS”-Disuc cinimidyl suberate; “BMH”-Bis maleimidohexane; “BSSS”-bis(sulfosuccinimidyl) suber ate; “DFDNB”-1,5-difluoro-2,4-dinitrobenzene; “DMA”-dimethyl adipimidate 2HCI; “DMP”-Dimethyl pimelimidate-2HCI; “DMS”-dimethyl suberimidate-2-HCl; “SPDPN-succinimidyl-3-(2-py ridylthio) propionate; “Sulfo-HSAB”-Sulfosuccinimidyl 4-(pazidophenyl) butyrate; “Sulfo-SAPB”-Sulfosuccinim idyl 4-(p-azidophenylbutyrate); “ASIB”-1-9p-azidosalicyla mido)-4-(iodoacetamido) butane; “ASBA”-4-(p-Azidosali-cylamido) butylamine.

Bifunctional chemical linkers include 4-succinimidyl-oxycarbonyl-C-(2-pyridyldithio) toluene; sulfosuccinimidyl-6-O-methyl-O-(pyridyldithiol)-toluamidohe-xanoate; N-succinimidyl-3 (2-pyridyldithio)-proprionate; succinimidyl-6-3 (-(−2-pyridyldithio)-proprionamidohexanoate; sulfosuccinimidyl-6-3 (-(−2-pyridyldithio)-propionamido hexanoate; 3-(2-pyridyldithio)-propionyl hydrazide, Ellman's reagent, dichlorotriazinic acid, S-(2-thiopyridyl)-Lcysteine, and the like. Further bifunctional linking molecules are disclosed in U.S. Pat. Nos. 5,349,066; 5,618,528; 4,569,789; 4,952,394; and 5,137,877, each of which is incorporated herein by reference in its entirety.

There are a large number of chemical cross-linking agents that are known to those skilled in the art and useful for cross-linking portions of a conjugate. For example, the cross-linking agents are heterobifunctional cross-linkers, which can be used to link molecules in a stepwise manner. Heterobifunctional crosslinkers provide the ability to design more specific coupling methods for conjugating, thereby reducing the occurrences of unwanted side reactions such as homo-protein polymers.

A wide variety of heterobifunctional cross-linkers are known in the art, including succinimidyl 4-(N-maleimidomethyl)cyclohexane-1-carboxylate (SMCC), m-Maleimidobenzoyl N-hydroxysuccinimide ester (MBS); N-succinimidyl (4-iodoacetyl)aminobenzoate (SIAB); succinimidyl 4-(p-ma-30 leimidophenyl) butyrate (SMPB); 1-ethyl-3-(3-dimethylaminopropyl) carbodiimide hydrochloride (EDC); 4-succinimidyloxycarbonyl-a-methyl-α-(2-pyridyldithio)-tolune (SMPT); N-succinimidyl 3-(2-pyridyldithio) propi onate (SPDP); succinimidyl 6-[3-(2-pyridyldithio) propi-onate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl subcrate (DSS), bismaleimidohexane (BMH) and dimethylpimelimidate: 2 HCl (DMP) are examples of useful homobifunctional cross linking agents, and bis-[B-(4-azidosalicylamido)ethyl]disul fide (BASED) and N-succinimidyl-6 (4′-azido-2′-nitrophe nylamino) hexanoate (SANPAH) are examples of useful photoreactive cross-linkers.

One class of heterobifunctional cross-linkers, included above, contain the primary amine reactive group, N-hydroxysuccinimide (NHS), or its water soluble analog N-hydroxysulfosuccinimide (sulfa-NHS). Primary amines (lysine epsilon groups) at alkaline pH's are unprotonated and react by nucleophilic attack on NHS or sulfa-NHS esters. This reaction results in the formation of an amide bond, and release of NHS or sulfa-NHS as a byproduct. Another reactive group useful as part of a hetero bifunctional cross-linker is a thiol reactive group. Common thiol reactive groups include maleimides, halogens, and pyridyl disulfides. Maleimides react specifically with free sulfhydryls (cysteine residues) in minutes, under slightly acidic to neutral (pH 6.5-7.5) conditions. Halogens (iodo acetyl functions) react with —SH groups at physiological pH's. Both of these reactive groups result in the formation of stable thioether bonds.

In embodiments, the universal antibody-oligonucleotide conjugate (AOC) provided herein comprises a linker attachment schema selected from the group consisting of: conjugation to exposed lysines, cysteine conjugation following modest reduction of 3E10, engineering free cysteines (e.g., at the C-terminus), site-specific transglutaminase linkage, wherein the linkage can further comprise click chemistry, and any combination thereof. In embodiments, an AOC provided herein further comprises a linker attachment schema comprising engineering free cysteines (e.g., at the C-terminus). In embodiments, an AOC provided herein, comprises an antibody that is cysteine-engineered at the site of linker attachment. In embodiments, an AOC provided herein, comprises a cleavable linker comprising a cleavable disulfide bond. In embodiments, an AOC provided herein comprises a linker wherein the linker is a hindered linker, wherein the linker comprises a carbon atom bearing a sulfur capable of forming a disulfide bond, and wherein the carbon atom is substituted with at least one substituent other than H. In embodiments, the substituent comprises a hydrocarbyl or a substituted hydrocarbyl moiety.

In some embodiments, the linker L is selected from:

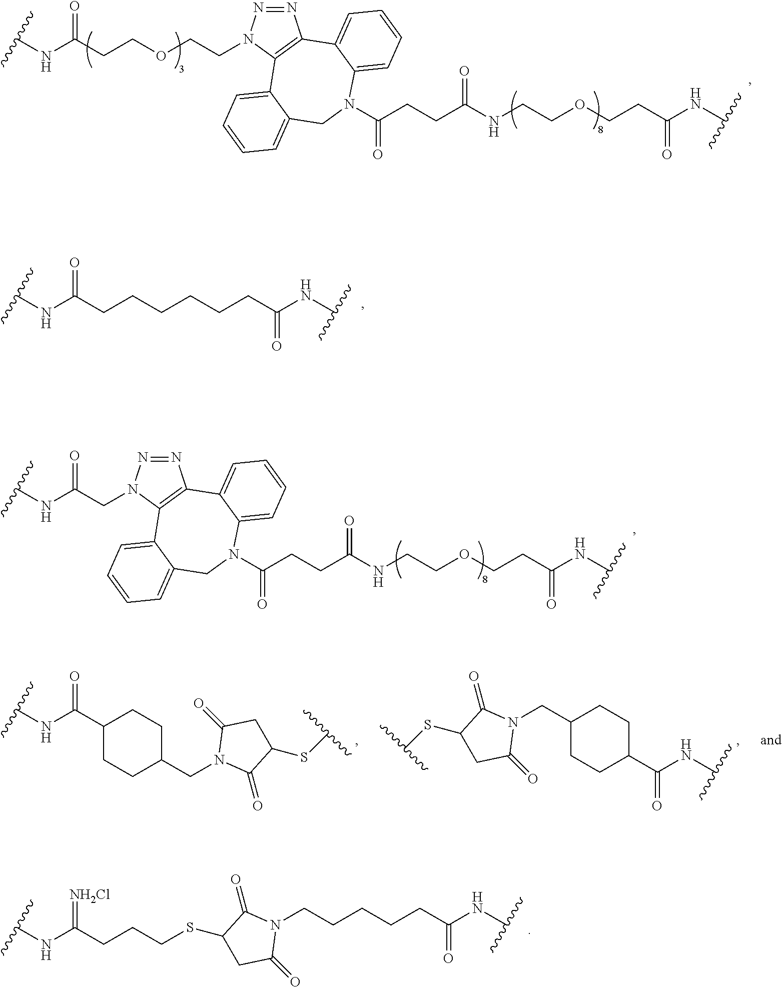

In some embodiments, the linker L is selected from:

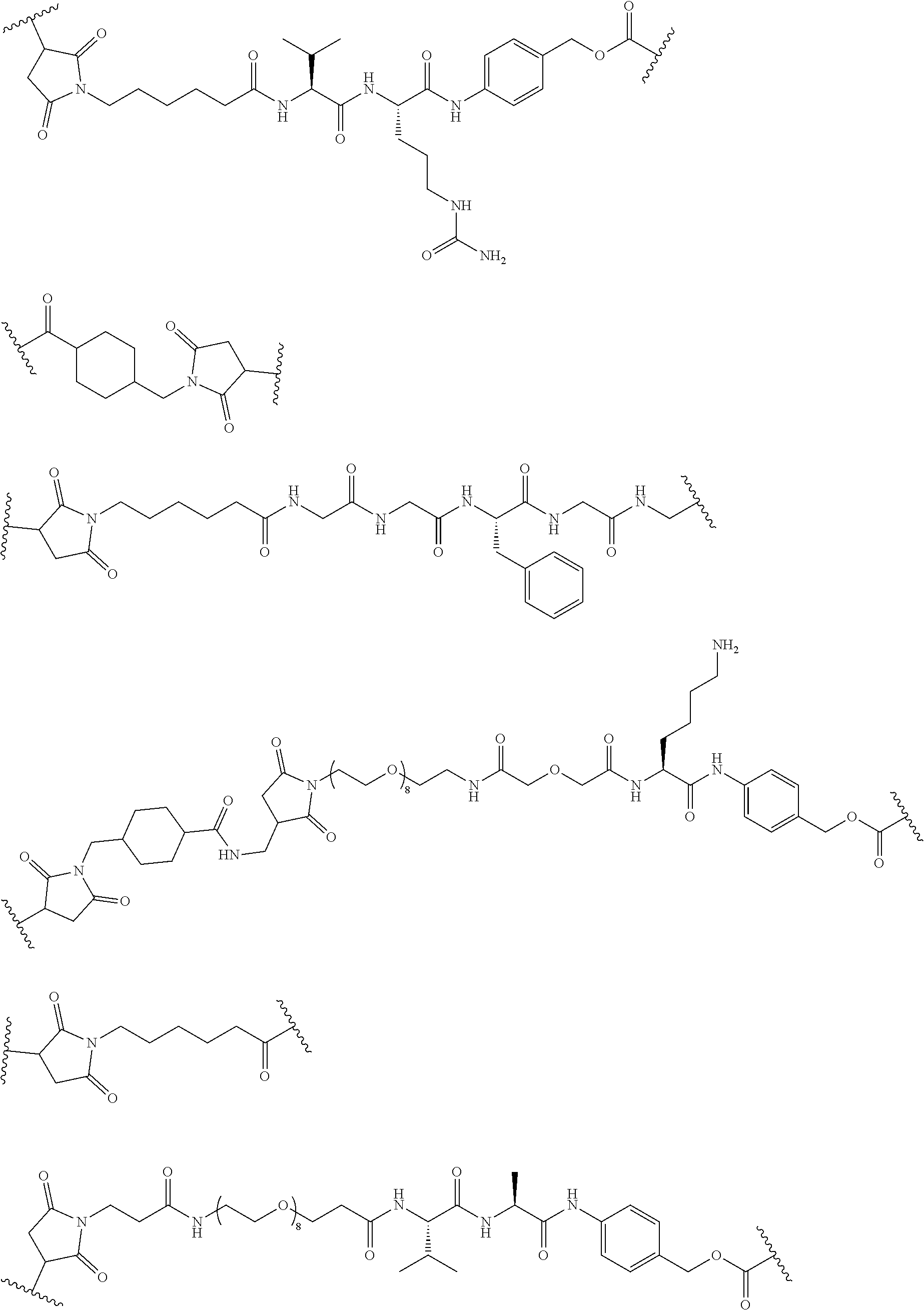

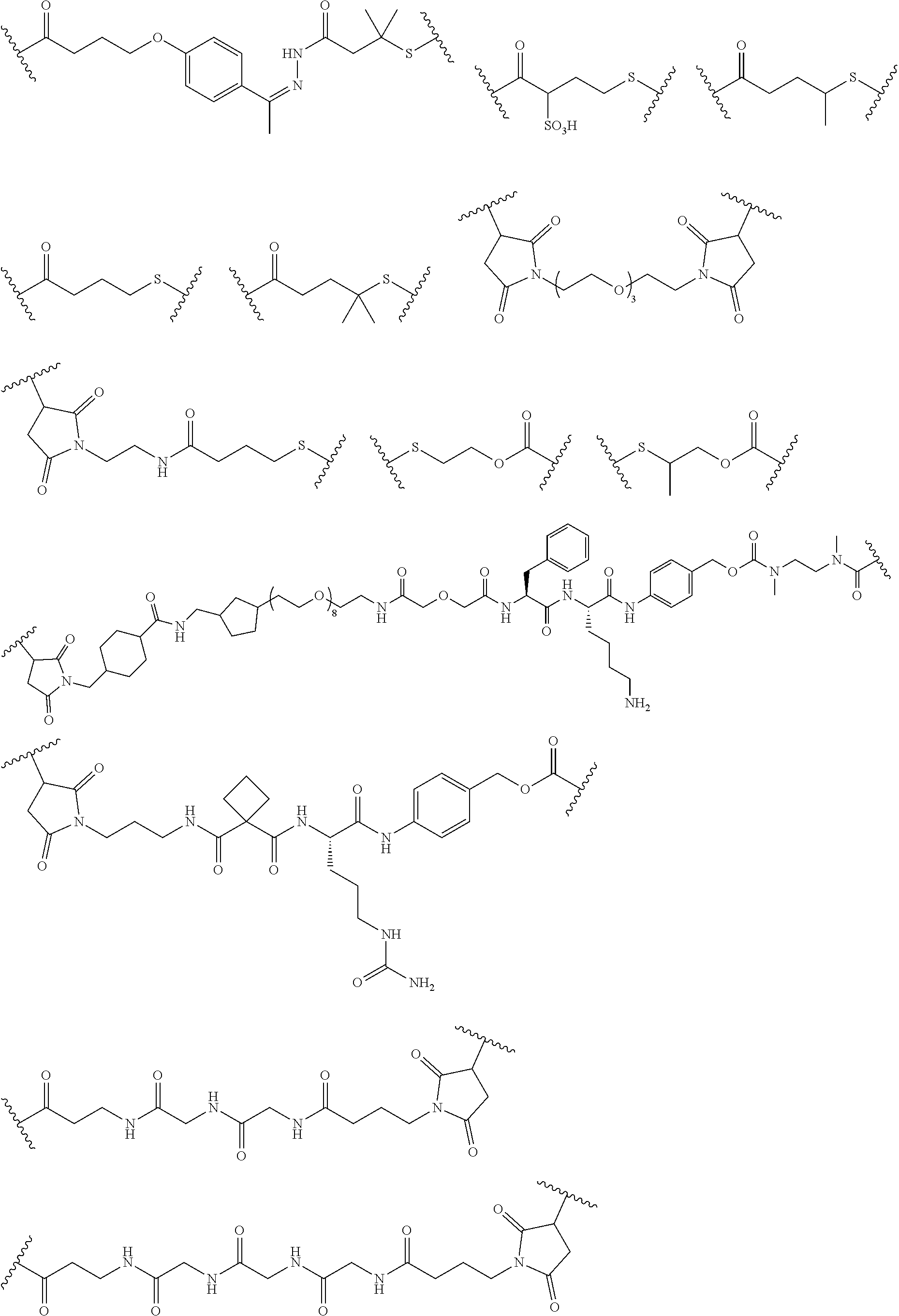

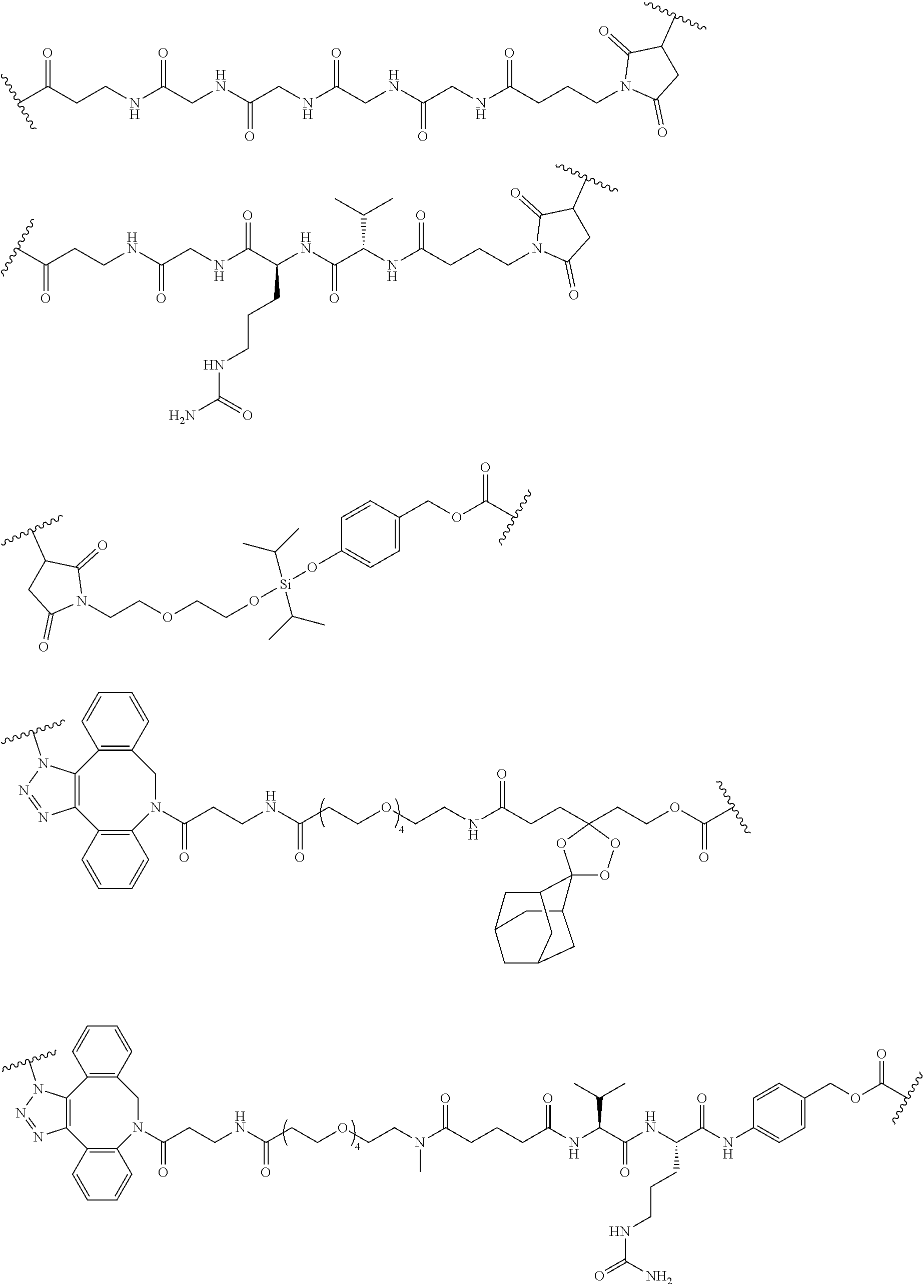

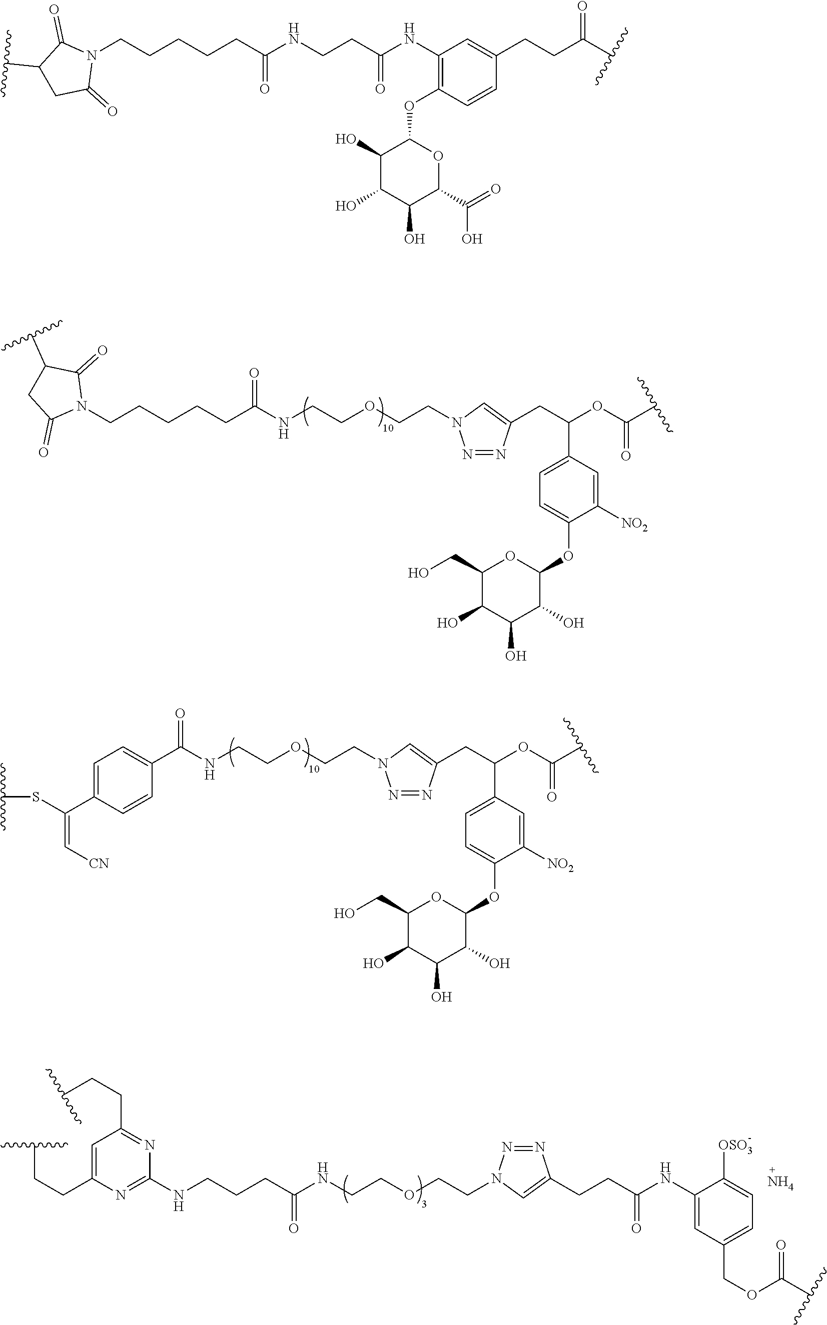

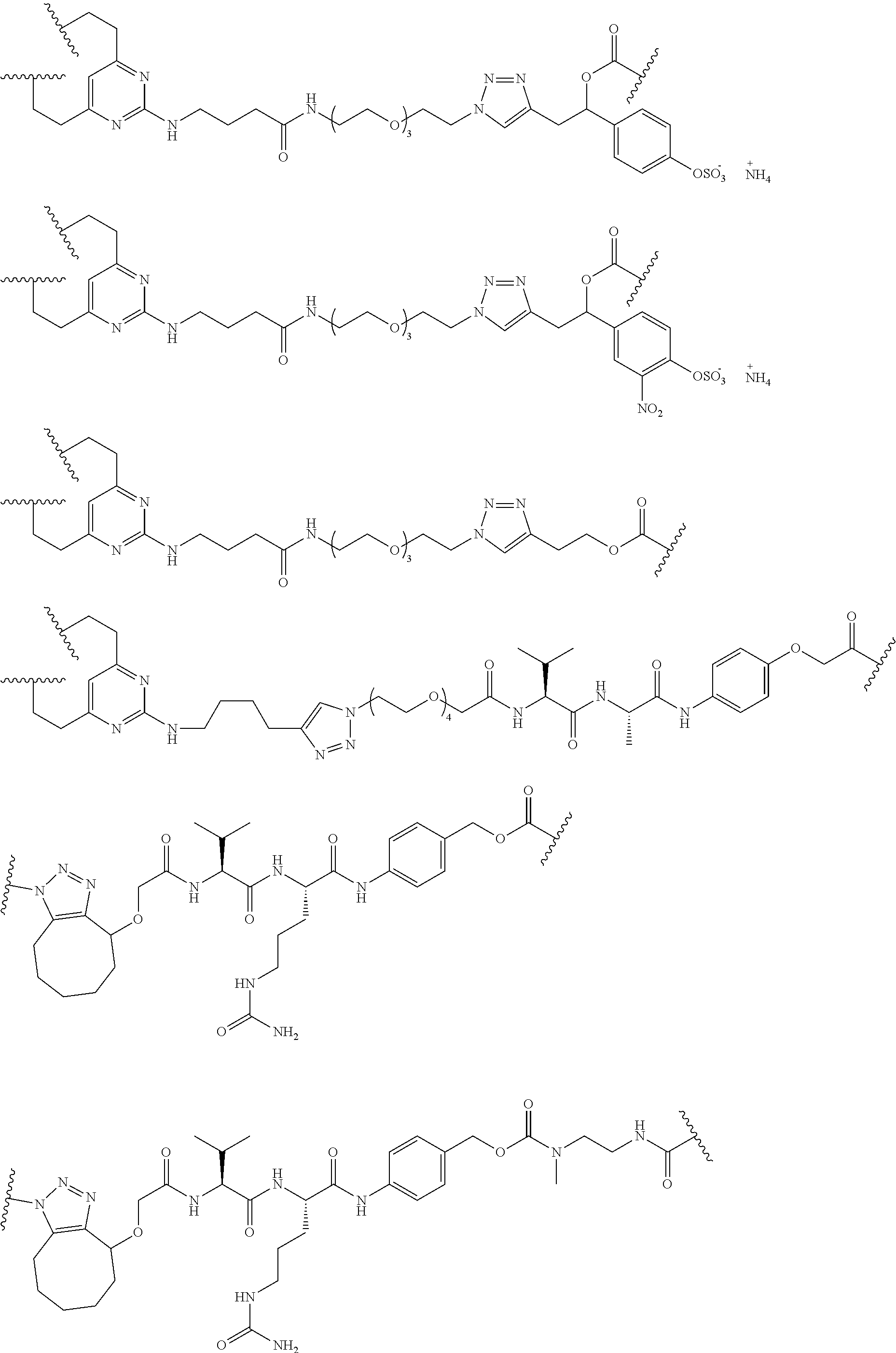

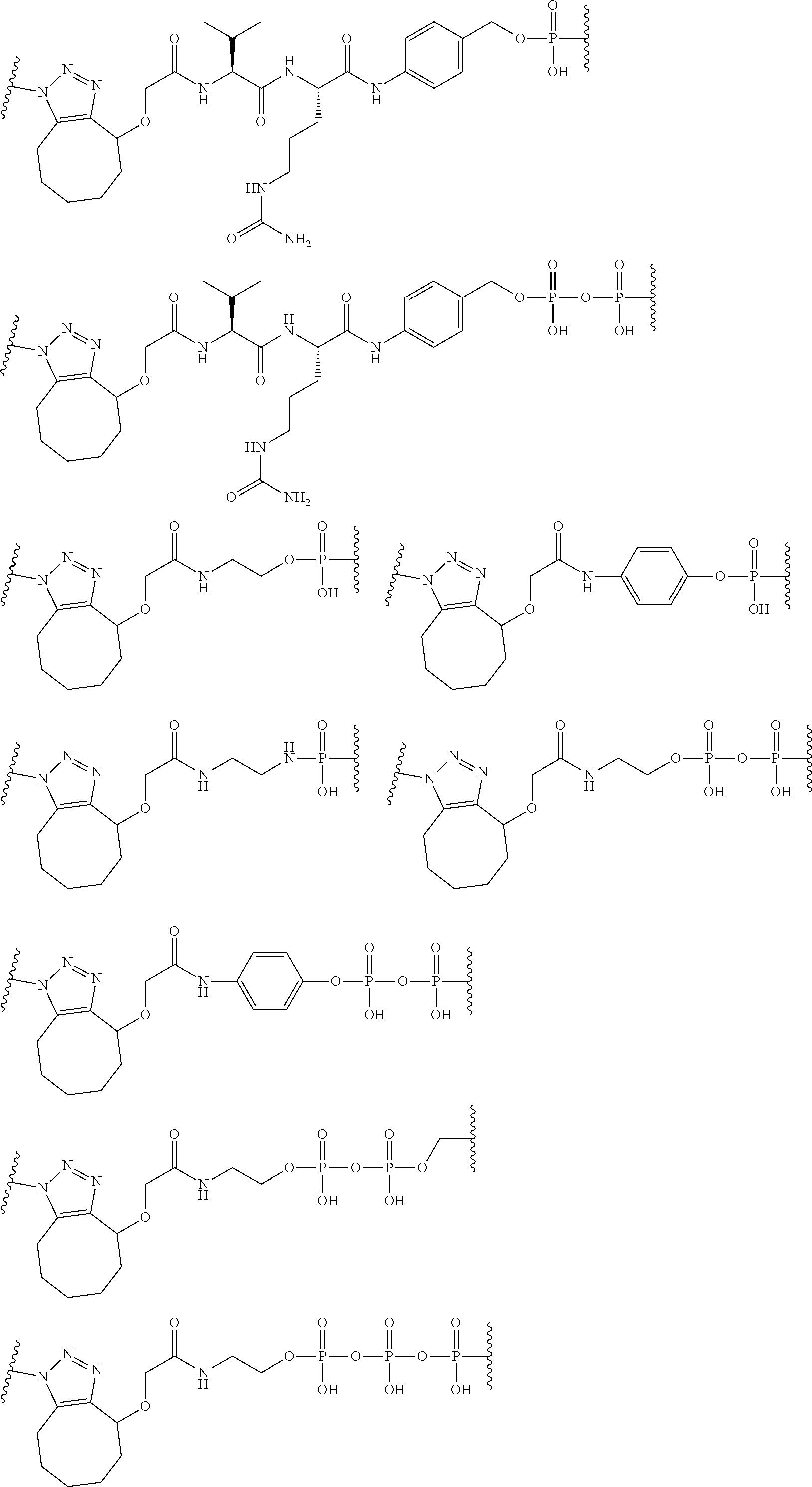

[chemical structure]

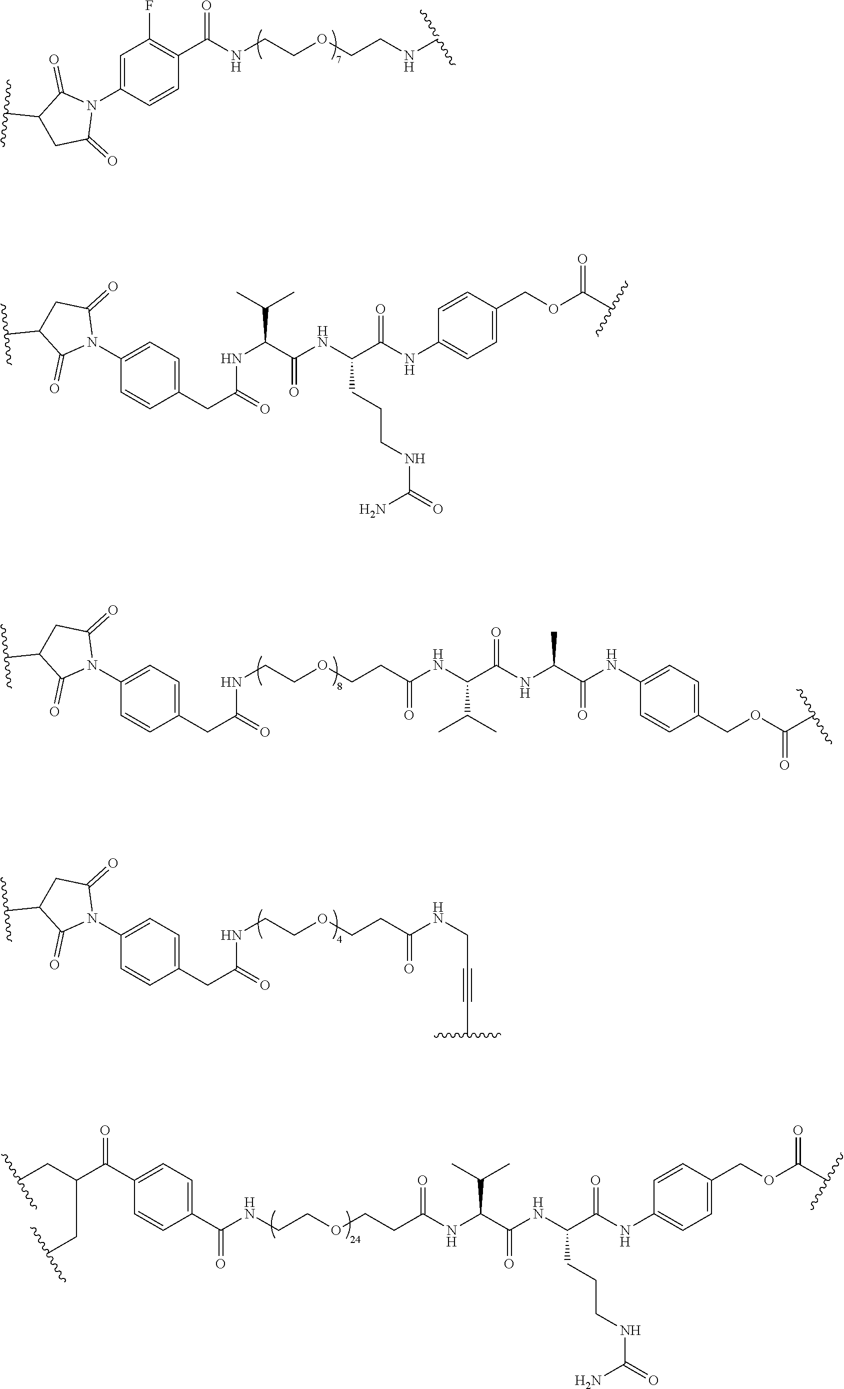

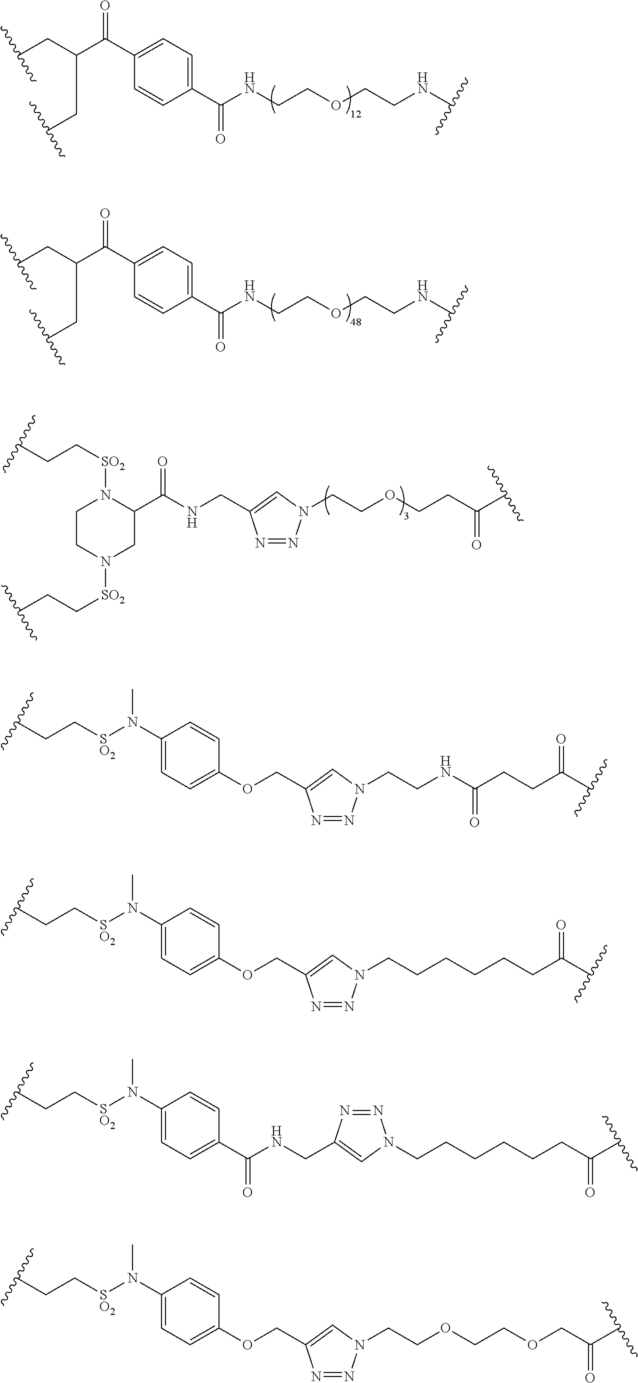

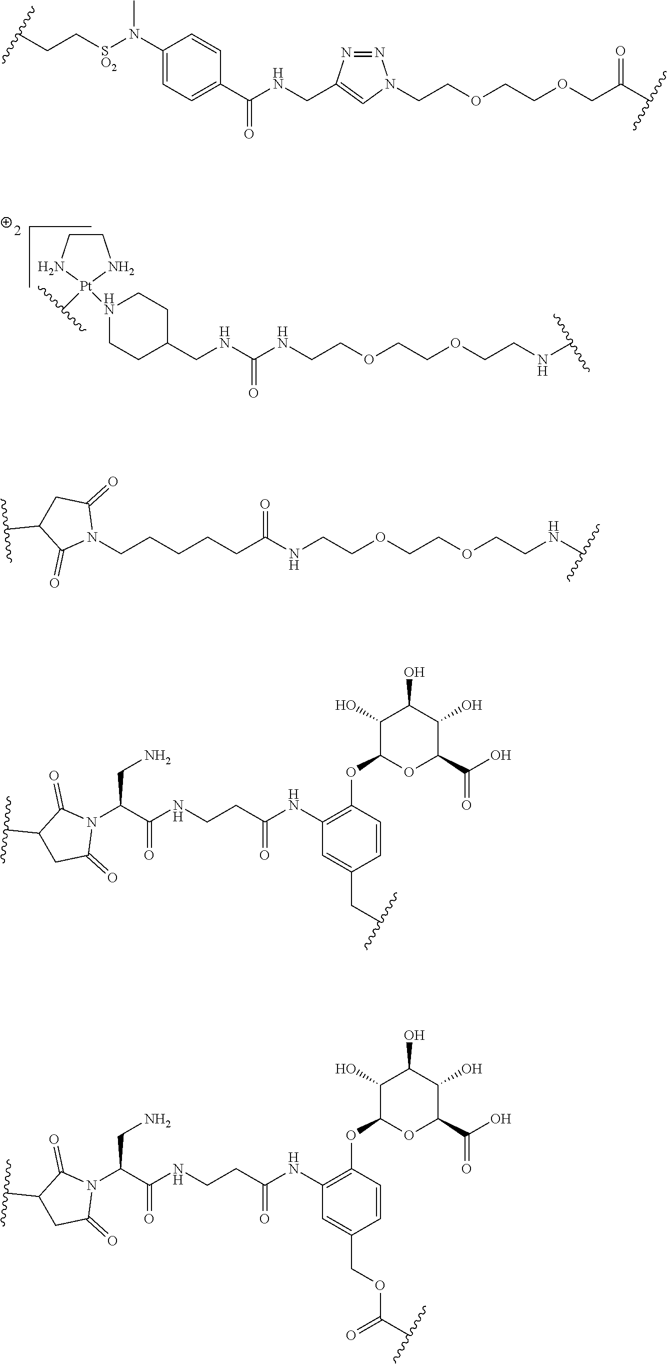

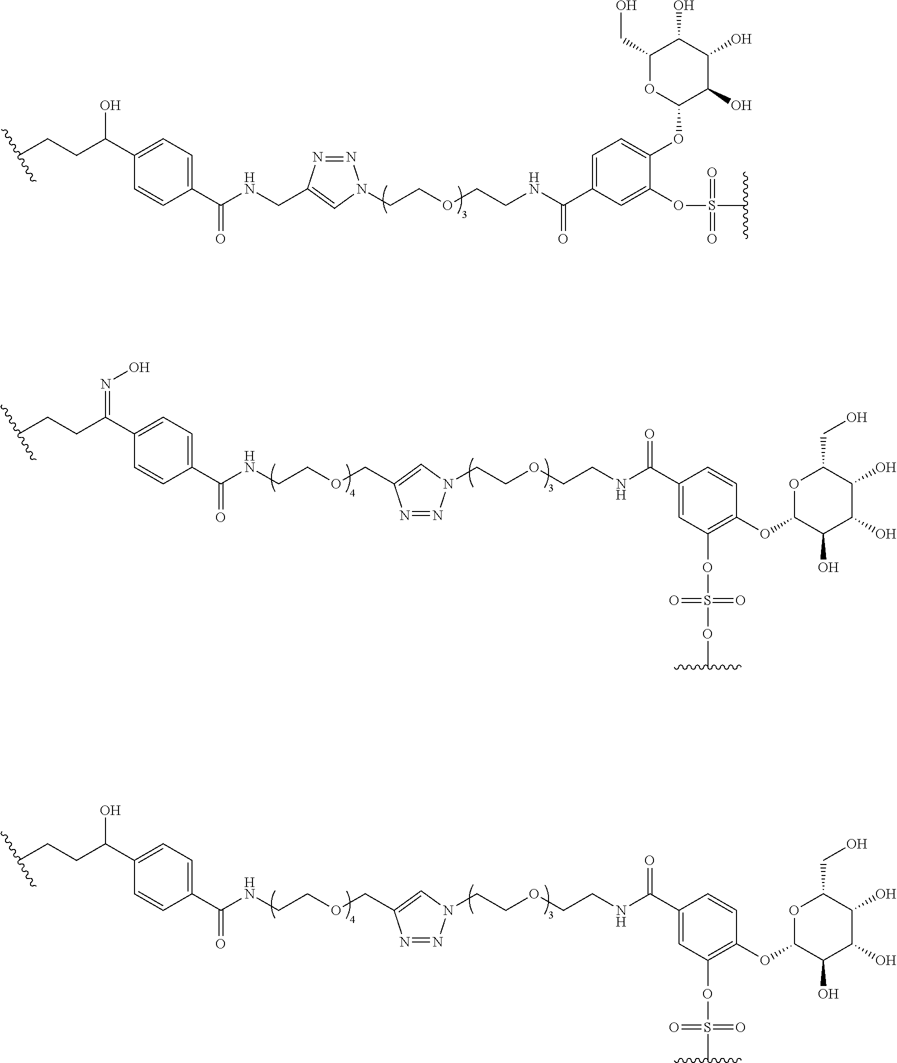

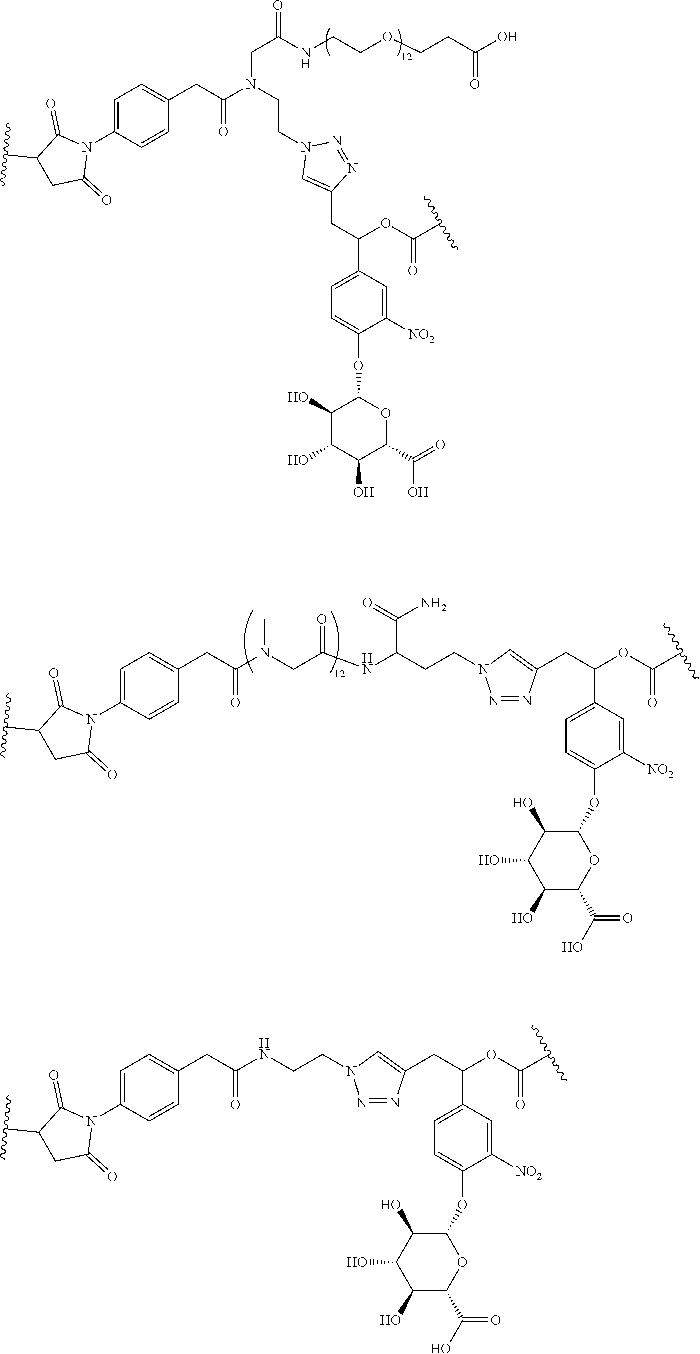

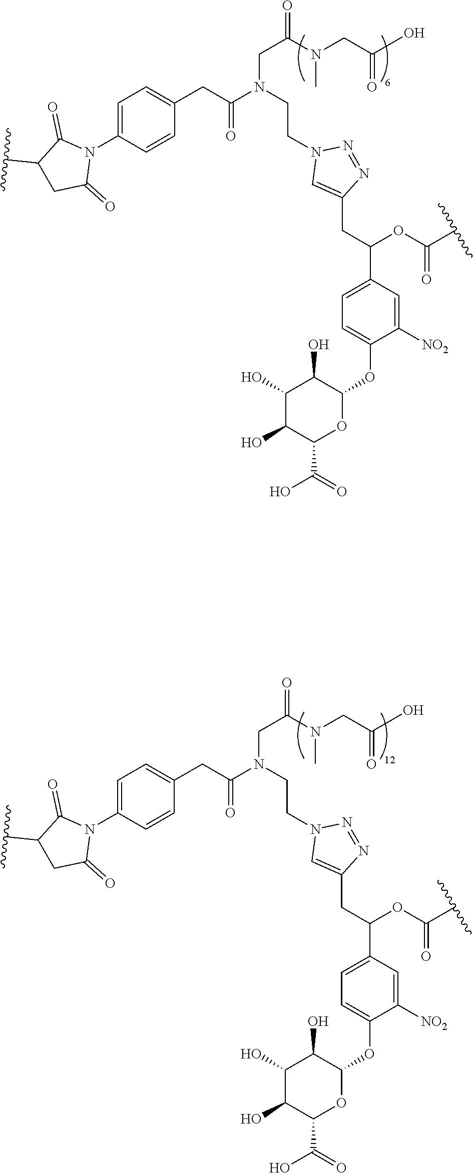

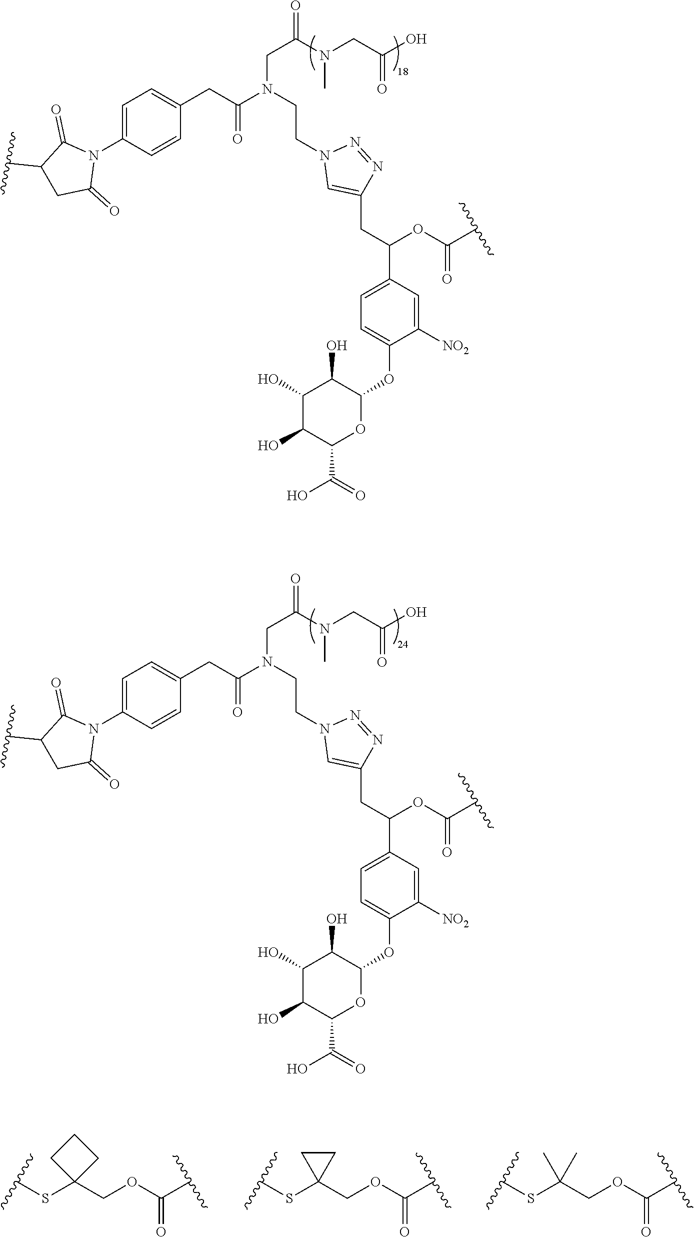

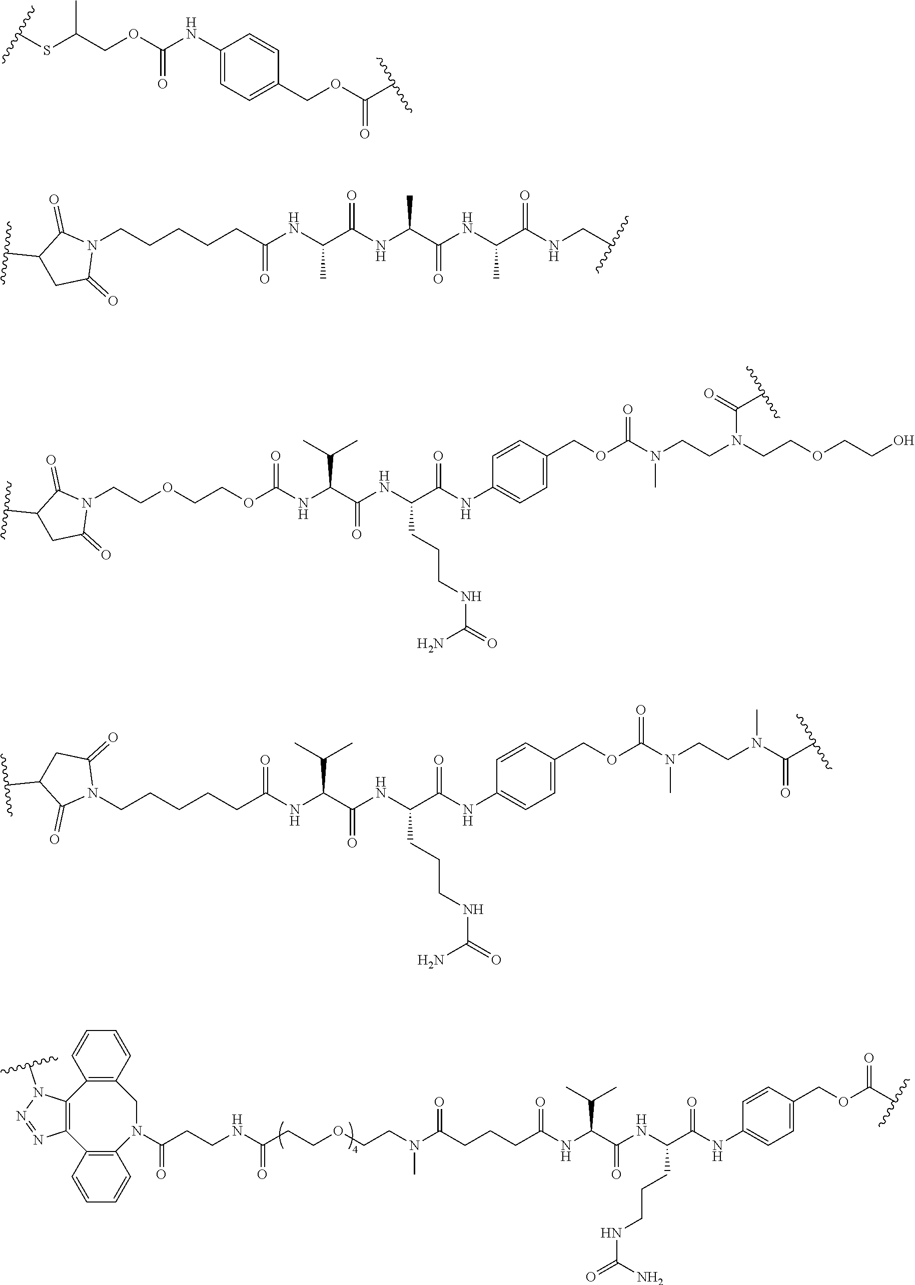

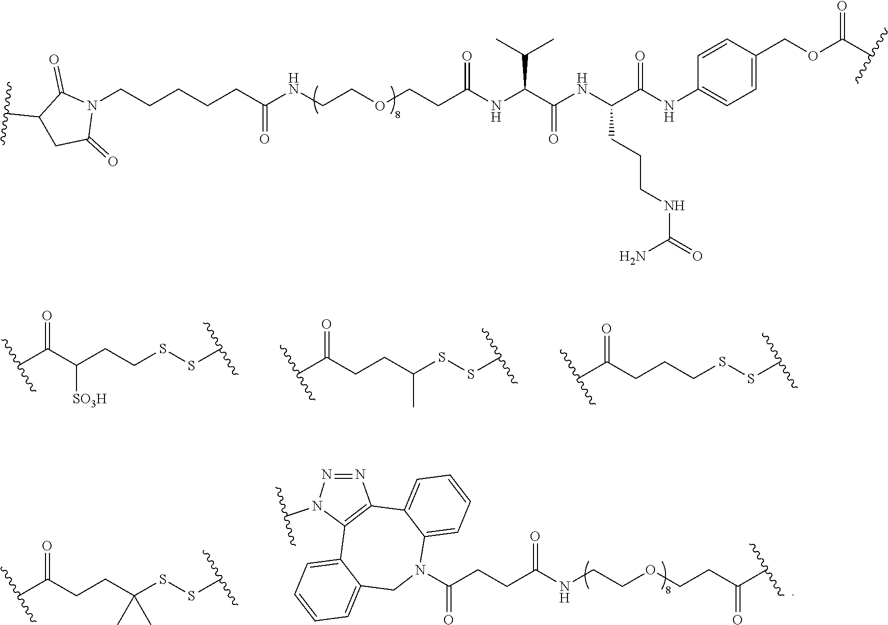

### C. Therapeutic Oligonucleotides

In embodiments, the present disclosure provides an antibody-oligonucleotide conjugate (AOC) having the formula A-(L-Pᵣ)_{q}, wherein: A is a 3E10 antibody or antigen-binding fragment thereof, L is a linker, P is an oligonucleotide moiety described herein, r is an integer from 1 to 4, and q is an integer from 1 to 16. In embodiments, P is a therapeutic oligonucleotide.

As used herein, the term “therapeutic oligonucleotide” refers to an oligonucleotide that has a biological, a cytotoxic, or a therapeutic effect in a cell. In embodiments, the therapeutic oligonucleotide is a functional nucleic acid, such as an oligonucleotide or a polynucleotide.

In some embodiments, a 3E10 antibody or antigen-binding fragment thereof described herein is used to deliver a gene-regulating polynucleotide that inhibits, reduces or silences expression a gene product that promotes cancer growth and/or progression, e.g., by targeting the gene or a transcript thereof. Non-limiting examples of gene-regulating polynucleotides include siRNAs and antisense oligonucleotides. For a review of the various types of gene-regulating polynucleotides that have been researched for therapeutic capability See, for example, Roberts T C, Langer R, Wood M J A, “Advances in oligonucleotide drug delivery,” Nat. Rev. Drug Discov., 19 (10): 673-94 (2020), the content of which is incorporated herein by reference, in its entirety, for all purposes.

In some embodiments, compositions described herein comprise a conjugate, comprising a 3E10 antibody or antigen-binding fragment thereof conjugated through a cleavable linker to an siRNA molecule that targets cMyc mRNA.

In other aspects, compositions described herein comprise a conjugate, comprising a 3E10 antibody or antigen-binding fragment thereof conjugated through a non-cleavable linker to an siRNA molecule that targets cMyc mRNA.

The Small interfering RNA (siRNA), also known as short interfering RNA or silencing RNA, is a class of double-stranded non-coding RNA molecules, typically from about 20 to about 27 nucleotides in length, from about 8 to about 50 nucleotides in length or from about 10 to about 30 nucleotides in length, and is processed within the RNA interference (RNAi) pathway.

In some aspects, an siRNA molecule comprises at least one 2′ modified nucleotide, at least one modified internucleotide linkage, at least one inverted abasic moiety, or any combination thereof.

Gene-regulating nucleic acid drugs such as siRNA can regulate post-transcriptional gene expression, and silence target genes, such as those involved in signaling pathways associated with cancer progression. See, for example, Zhou et al., Delivery of nucleic acid therapeutics for cancer immunotherapy, Medicine in Drug Discovery, Mar. 24, 2020; and Dahlman et al., In vivo endothelial siRNA delivery using polymeric nanoparticles with low molecular weight, Nature Nanotechnol. 2014; 9 (8): 648-55, the disclosures of which are incorporated herein by reference in their entireties for all purposes.

Thus, siRNAs may be used for modulating the expression of immune checkpoint molecules, such as those described herein, by regulating the post-translational gene expression and/or silencing corresponding genes. Similarly, siRNAs can be used for indirectly regulating the activity of immune checkpoint molecules by modulating the expression of agonists or inhibitors of immune checkpoint molecules.

Accordingly, in some embodiments, compositions for treatment of cancer described herein include a conjugate, comprising a 3E10 antibody or antigen-binding fragment thereof conjugated through a cleavable linker to an siRNA molecule that targets cMyc mRNA.

In some other embodiments, compositions for treatment of cancer described herein includes a conjugate, comprising a 3E10 antibody or antigen-binding fragment thereof conjugated through a non-cleavable linker to an siRNA molecule that targets cMyc mRNA.

The siRNAs can similarly be used for silencing genes regulating tumor growth or angiogenesis. For example, siRNAs have been used to target vascular endothelial growth factor (VEGF) and kinesin spindle protein (KSP) (for solid tumors, e.g., liver metastasis from colon cancer). Other genetic targets that can be silenced using siRNAs include, but are not limited to, genes encoding protein kinase N3 (PKN3) (e.g., for metastatic pancreatic cancer), M2 subunit of ribonucleotide reductase (RRM2) (e.g., for solid tumors), Myc oncoprotein (e.g., for hepatocellular carcinoma), ephrin type-A receptor 2 (EphA2) (e.g., for advanced cancers), and KRAS G12D mutation (e.g., for advanced pancreatic cancers). See, for example, Chalbatani G. M., et al., Int J Nanomedicine, 14:3111-28 (2019), the disclosure of which is incorporated herein by reference in its entirety for all purposes.

Other non-limiting examples of siRNA and associated cancer types being studied are provided in Table 1A below. See, for example, Xiong H. et al., Int. J. Mol. Sci., 22 (7): 3295 (2021), the disclosure of which is incorporated herein by reference in its entirety for all purposes.

TABLE 1A
Example siRNA and associated cancer types

| Drug/Therapeutic | Target | Cancer |
|---|---|---|
| KRAS G12D siRNA | KRASG12D | Pancreatic cancer |
| EphA2-targeting DOPC- | EPHA2 | Solid tumors |
| encapsulated siRNA | | |
| APN401 | CBLB | Brain cancer, melanoma, pancreatic cancer, |
| | | renal cell cancer |
| Proteosome siRNA and tumor | LMP2, | Melanoma |
| antigen RNA-transfected | LMP7, | |
| dendritic cells | MECL1 | |
| TKM-080301 | PLK1 | Cancer with hepatic metastases, liver cancer, |
| | | hepatocellular cancer, adrenocortical cancer |
| Atu027 | PNK3 | Solid tumors, pancreatic cancer |
| DCR-MYC | MYC | Solid tumors, hepatocellular cancer |
| CALAA-01 | M2 subunit of | Solid tumors |
| | ribonucleotide | |
| | reductase 2 | |
| siG12D LODER | KRASG12D | Pancreatic cancer |
| ARO-HIF2 | HIF2A | Clear cell renal cell carcinoma |
| SV40 vectors carrying siRNA | Unknown | Chronic myeloid leukemia |

Non-limiting example sequences of siRNA oligonucleotide molecules for use in treating various disorders are provided in Table 1B below.

TABLE 1B
Example siRNA oligonucleotide sequences and associated disorders

| | Target | | | | SEQ ID | |
|---|---|---|---|---|---|---|
| Disease | Gene | Name | Strand | Sequence | NO | Reference |
| Hereditary | TTR | Patisiran | S | GUAACCAAGAGU | 152 | U.S. |
| transthyretin | (NM_ | | | AUUCCAUDTDT | | 10,060,921 |
| amyloidosis | 000371.4) | | AS | AUGGAAUACUCU | 153 | |
| | | | | UGGUUACDTDT | | |
| | | Vutrisiran | S | UCUUGGUUACAU | 154 | U.S. |
| | | | | GAAAUCCCAUC | | 2020/0248180 |
| | | | AS | UGGGAUUUCAUG | 155 | |
| | | | | UAACCAAGA | | |
| Acute | IKBKAP | Givosiran | S | CAGAAAGAGUGU | 156 | U.S. |
| Hepatic | (NM_ | | | CUCAUCUUA | | 11,091,759 |
| Porphyria | 003640.5) | | AS | AAUGAUGAGACA | 157 | |
| | | | | CUCUUUCUGGU | | |
| Primary | HAO1 | Lumasiran | S | UAUAUUUCCAGG | 158 | U.S. |
| hyperoxaluria | (NM_ | | | AUGAAAGUCCA | | 11,261,447 |
| type 1 | 017545.3) | | AS | GACUUUCAUCCU | 159 | |
| | | | | GGAAAUAUA | | |
| Hypercholes | PCSK9 | Inclisiran | S | ACAAAAGCAAAA | 160 | U.S. |
| terolemia | (NM_ | | | CAGGUCUAGAA | | 2023/0235020 |
| | 174936.4) | | AS | CUAGACCUGUTU | 161 | |
| | | | | UGCUUUUGUN | | |
| Primary | LDHA | Nedosiran | S | AUGUUGUCCUUU | 162 | U.S. |
| Hyper- | (NM_ | | | UUAUCUGAGCAG | | 11,661,604 |
| oxaliuria | 001165416.2) | | | CCGAAAGGCUGC | | |
| Type 3 | | | AS | UCAGAUAAAAAG | 163 | |
| | | | | GACAACAUGG | | |
| Hemophelia | SERPIN | Fitusiran | S | GGUUAACACCAU | 164 | U.S. |
| A/B | C1 | | | UUACUUCAA | | 2021/0393669 |
| | (NM_ | | AS | UUGAAGUAAAUG | 165 | |
| | 000488.4) | | | GUGUUAACCAG | | |
| Acute | P53 | Teprasiran | S | GAGAAUAUUUCA | 166 | U.S. |
| kidney | (NM_ | | | CCCUUCA | | 11,015,198 |
| injury | 001276696.3) | | AS | UGAAGGGUGAAA | 167 | |
| | | | | UAUUCUC | | |
| Nonarteritic | CASP2 | Cosdosiran | S | AGGAGUUCCACA | 168 | U.S. |
| Anterior | (NM_ | | | UUCUGGC | | 11,015,198 |
| Ischemic | 032982.4) | | AS | GCCAGAAUGUGG | 169 | |
| Optic | | | | AACUCCU | | |
| Neuripathy | | | | | | |
| Ocular pain | TRPV4 | Tivanisirain | S | UGAAGUAGAAGA | 170 | U.S. |
| | (NM_ | | | UGCGCUU | | 11,015,198 |
| | 001177433.1) | | AS | AAGCGCAUCUUC | 171 | |
| | | | | UACUUCA | | |
| alpha-1 | SERPIN | Belcesiran | S | AAACCCUUUGUC | 172 | U.S. |
| antitrypsin | A1 | | | UUCUUAAAGCAG | | 2022/0380761 |
| deficiency | (NM_ | | | CCGAAAGGCUGC | | |
| associated | 000295.5) | | AS | UUUAAGAAGACA | 173 | |
| liver disease | | | | AAGGGUUUGG | | |
| Glaucoma, | ADRB2 | Bamosiran | S | CUGGAUCACAUG | 174 | U.S. |
| Ocular | (NM_ | | | CACAAUGTT | | 10,011,832 |
| hypertension | 000024.6) | | AS | CAUUGUGCAUGU | 175 | |
| | | | | GAUCCAGTT | | |
| Hemolytic | C5 | Cemdisiran | S | AAGCAAGAUAUU | 176 | U.S. |
| uremic | (NM_ | | | UUUAUAAUAN | | 2021/0046182 |
| syndrome, | 001735.3) | | AS | UAUUAUAAAAAU | 177 | |
| IgA | | | | AUCUUGCUUUUT | | |
| nephropathy, | | | | T | | |
| Paroxysmal | | | | | | |
| nocturnal | | | | | | |
| hemoglobin | | | | | | |
| uria, | | | | | | |
| Myasthenia | | | | | | |
| gravis | | | | | | |

In some embodiments, compositions described herein comprise a conjugate, comprising a 3E10 antibody or an antigen-binding fragment thereof conjugated through a cleavable linker to an antisense oligonucleotide.

In other aspects, compositions described herein comprise a conjugate, comprising a 3E10 antibody or an antigen-binding fragment thereof conjugated through a non-cleavable linker to an antisense oligonucleotide.

Antisense oligonucleotides (ASOs) are short, synthetic, chemically modified chains of nucleotides that have the potential to target any gene product of interest. In embodiments, an ASO is a single-stranded sequence complementary to the sequence of the target gene's transcribed messenger RNA (mRNA) within a cell. See, for example, Rinaldi et al., “Antisense oligonucleotides: the next frontier for treatment of neurological disorders,” Nat. Rev. Neurol. 2018; 14 (1): 9-21; Bennett, Therapeutic Antisense Oligonucleotides Are Coming of Age. Ann. Rev. Med. 2019; 70:307-321, the disclosures of which are incorporated herein by reference in their entireties for all purposes. An ASO targets the corresponding mRNA to degrade the targeted complex by mechanisms such as endogenous cellular RNase H.

One example of an ASO being used for cancer therapy is an ASO targeting CD39 mRNA, resulting in an increase in CD8+ T cell proliferation, thereby improving antitumor immune responses. See, for example, Zhou, et al. Delivery of nucleic acid therapeutics for cancer immunotherapy. Medicine in Drug Discovery, 6 (2020) 100023, the disclosure of which is incorporated herein by reference in its entirety for all purposes.

Other non-limiting examples of ASOs and the cancers they are associated with are provided in Table 2.

TABLE 2
Example ASOs being studied for treatment of cancers
(see, e.g., Int. J. Mol. Sci 22 (2021) 3295):

| Drug/Therapeutic | Target | Cancer |
|---|---|---|
| AEG35156 | AEG35156 | Hepatocellular Cancer, Pancreatic Cancer, Breast |
| | | Cancer, Non-Small Cell Lung Cancer, Leukemia, |
| | | Lymphoma |
| Apatorsen (OGX-427) | HSP27 | Urologic Cancer, Bladder Cancer, Prostate Cancer, |
| | | Urothelial Cancer, Non-Small Cell Lung Cancer |
| ARRx (AZD5312) | AR | Prostate cancer |
| AZD4785 | KRAS | Non-Small Cell Lung Cancer |
| AZD8701 | FOXP3 | Advanced cancer |
| AZD9150 | STAT3 | Bladder cancer, lymphoma, malignancies |
| BP1001 | GRB2 | Ph1 Positive Leukemia, Acute Myeloid Leukemia, |
| | | Chronic Myelogenous Leukemia |
| Cenersen (EL625) | TP53 | Acute Myelogenous Leukemia, lymphoma |
| CpG 7909 | TLR9 | Melanoma, Breast Cancer, Renal Cancer, |
| (PF03512676) | | Lymphoma, Non-Small Cell Lung Cancer, |
| | | Esophageal Cancer, Prostate Cancer |
| CpG ODN (GNKG168) | TLR9 | Leukemia |
| CpG Oligonucleotide | TLR9 | Breast cancer |
| CpG-ODN | TLR9 | Glioblastoma |
| Custirsen (OGX-011) | ApoJ | Prostate cancer, breast cancer, non-small cell lung |
| | | cancer |
| Danvatirsen (AZD9150, | STAT3 | Advanced cancers |
| ISIS STAT3Rx) | | |
| EGFR Antisense DNA | EGFR | Head and Neck Squamous Cell Cancer, Gastric |
| | | Cancer, Ovarian Cancer, Prostate Cancer |
| EZN-2968 | HIF1A | Hepatocellular cancer, lymphoma |
| (RO7070179, SPC2968) | | |
| G4460 | CMYB | Leukemia, hematologic malignancies |
| IGF-1R/AS ODN | IGF1 | Glioma |
| IGV-001 containing | IGF1R | Glioblastoma |
| autologous GBM cells | | |
| treated with antisense | | |
| oligonucleotide (IMV- | | |
| 001) | | |
| IMO-2055 (EMD | TLR9 | Renal cell cancer, colorectal cancer, non-small cell |
| 1201081) | | lunch cancer, head and neck cancer |
| ION251 | IRF4 | Myeloma |
| ION537 | YAP1 | Advanced solid tumors |
| ISIS 183750(ISIS- | EIF4E | Castrate-resistant prostate cancer, non-small cell lung |
| EIF4ERx, LY2275796) | | cancer, colorectal cancer |
| ISIS 2503 | HRAS | Colorectal cancer, pancreatic cancer |
| ISIS 5132 | CRAF | Ovarian cancer |
| L-Bcl-2 antisense | BCL2 | Advanced lymphoid malignancies |
| oligonucleotide | | |
| LErafAON | CRAF | Cancers |
| Lucanix | TGFB2 | Non-small cell lung cancer |
| LY2181308 | BIRC5 | Non-small cell lung cancer |
| LY900003 (ISIS 3521, | PKCA | Melanoma, lung cancer, non-small cell lung cancer, |
| Affinitak) | | breast cancer |
| MTL-CEBPA | CEBPA | Hepatocellular cancer |
| Oblimersen (G3139) | BCL2 | Cancers |
| OGX-427 | HSP27 | Cancers |
| PNT2258 | BCL2 | Prostate cancer, lymphoma, melanoma |
| SPC2996 | BCL2 | Chronic lymphocytic leukemia |
| TGF 2 Antisense- | TGFB2 | Advanced cancers |
| GMCSF Gene Modified | | |
| Autologous Tumor Cell | | |
| (TAG) Vaccine | | |
| SD-101 | TLR9 | Cancers |
| Trabedersen (AP 12009, | TGFB2 | Glioblastoma, anaplastic astrocytoma, pancreatic |
| OT-101) | | cancer, melanoma, colorectal cancer |
| VEGF-Antisense | VEGF | Mesothelioma |
| Oligonucleotide | | |

In some embodiments, the binding (e.g., hybridization) of the oligonucleotide, e.g., siRNA or ASO of an antibody-oligonucleotide conjugate, to target mRNA leads to degradation of the target mRNA or blocks translation of the target mRNA. In some embodiments, the binding of the oligonucleotide to the target mRNA creates a duplex nucleic acid molecule, which then recruits an endogenous nuclease for degradation of the mRNA. In some embodiments, the oligonucleotide has a stretch of DNA nucleotides sufficient to recruit RNaseH, and thereby trigger degradation of the target mRNA. For example, the oligonucleotide may have a stretch (e.g., a central stretch) of at least 6 or at least 8 DNA nucleotides, and which is optionally a stretch of 9 or 10 DNA nucleotides. In some embodiments, one or more DNA and/or RNA nucleotides comprise a 2′ chemical modification independently selected from 2′-Fluoro, 2′-Methyl, and 2′-Ethyl. As such, in some embodiments a siRNA or an ASO may have a design. For example, the antisense oligonucleotide may be a gapmer (i.e., have a gapmer design) having a 5′ and a 3′ segment, each of the 5′ and 3′ segments independently being from 2 to 6 nucleotides or from 2 to 4 nucleotides, and where the 5′ and 3′ segments do not contain DNA nucleotides. In some embodiments, the gapmer is a 3-8-3 gapmer, having a central block of DNA nucleotides and 5′ and 3′ segments of 3 RNA nucleotides each. In other embodiments, the gapmer is a 2-10-2 gapmer, having a central block of 10 DNA nucleotides and a 5′ and 3′ segments of 2 RNA nucleotides each. Additional non-limiting examples of gapmer designs are described in U.S. Patent Appl. Publ. No. 2012/0322851. In some embodiments, one or more nucleotides of the 5′ segment and the 3′ segment comprise 2′-O substituents, optionally where all of the nucleotides of the 5′ segment and the 3′ segment comprise 2′-O substituents. Exemplary 2′-O substituents are independently selected from 2′-O methyl, 2′-O ethyl, 2′-O methoxyethyl (MOE), and a bridged nucleotide (e.g., a locked or bi-cyclic nucleotide) having a 2′ to 4′ bridge. In some embodiments, the bridged nucleotide has a methylene bridge (LNA) or a constrained ethyl bridge (cEt). For further examples of 2′ modified nucleosides, please see e.g. Freier & Altmann; Nucl. Acid Res., 1997, 25, 4429-4443 and Uhlmann; Curr. Opinion in Drug Development, 2000, 3 (2), 293-213, and Deleavey and Damha, Chemistry and Biology 2012, 19, 937.

The term “gapmer” refers to an oligonucleotide having a central block of deoxynucleotides (also referred to herein as “DNA nucleotides”) with 5′ and 3′ segments (of at least 2 nucleotides) of RNA nucleotides. As used herein, the term “DNA nucleotide” refers to a nucleotide that is not an RNA nucleotide. DNA nucleotides typically have a 2′ H, but may alternatively have various 2′ chemical modifications, including 2′-halo and 2′-lower alkyl (e.g., C1-4). In some embodiments, the 2′ chemical modifications of DNA nucleotides are independently selected from 2′-Fluoro, 2′-Methyl, and 2′-Ethyl.

In some embodiments, the oligonucleotide has a modified backbone or modified internucleotide linkages. The term “internucleotide linkage” refers to the linkage between two adjacent nucleosides in a polynucleotide molecule. Naturally, the internucleotide linkage is a phosphodiester bond that forms between two oxygen atoms of the phosphate group and an oxygen atom of the sugar (either at 3′ or 5′ position) to form two ester bonds bridging between the two adjacent nucleosides. Modification of the internucleotide linkage may provide different characteristics, including but not limited to enhanced stability. For example, phosphorothioate or phosphorodithioate linkages increase the resistance of the internucleotide linkage to nucleases. Another example is phosphoacetate linkage (PACE), which improves transfection characteristics and enhances nuclease resistance. Internucleotide linkages and oligonucleotide backbone modifications which may be employed in the oligonucleotides of the present description include, but are not limited to, phosphodiester, phosphorothioate, phosphorodithioate, methylphosphonate, alkylphosphonate, alkylphosphonothioate, phosphotriester, phosphoramidate, phosphoramidite, phosphorodiamidate, siloxane, carbonate, carboalkoxy, acetamidate, carbamate, morpholino, peptide nucleic acid, borano, thioether, bridged phosphoramidate, bridged methylene phosphonate, bridged phosphorothioate, and sulfone internucleoside linkages. In some embodiments, the oligonucleotide comprises one or more phosphorothioate or phosphorodithioate nucleotides.

In some embodiments, the oligonucleotide comprises one or more phosphorothioate or phosphorodithioate internucleotide linkages. In some embodiments, phosphorothioate or phosphorodithioate bonds can be introduced between the last three to five nucleotides at the 5′- and/or 3′-end of the oligonucleotide to reduce exonuclease degradation. In some embodiments, the oligonucleotide has a combination of phosphodiester and phosphorothioate/phosphorodithioate linkages. In some embodiments, the oligonucleotide contains at least two, at least three, at least four, at least five, at least six, at least seven, at least eight, at least nine, or at least ten phosphorothioate or phosphorodithioate internucleotide linkages. In some embodiments, the oligonucleotide comprises substantially alternating phosphodiester and phosphorothioate internucleotide linkages. In some embodiments, the oligonucleotide is fully phosphorothioate/phosphorodithioate linked (i.e., all bonds are either phosphorothioate or phosphorodithioate).

In some embodiments, the oligonucleotide may contain one or more modified bases. In some embodiments, cytosine is replaced with 5-methylcytosine, which may enhance base pairing. Other modified bases (particularly of cytosine or guanine) can be employed to reduce immunogenicity, where needed. Other modified bases are described in U.S. Pat. No. 10,064,959, which is hereby incorporated by reference. In various embodiments, cytidine nucleobases in the oligonucleotide are 5-methyl cytidine. Thus, it will be understood by the skilled person that where a sequence includes a cytidine nucleobase (“C”), the term includes 5-methyl C. Further, it is understood that where uracil bases are included in a sequence, the term “U” includes pseudouridine and N1-methylpseudouridine.

### 1. Example ASOs for Exon Skipping

In embodiments, the present disclosure provides an antibody-oligonucleotide conjugate (AOC) having the formula A-(L-Pᵣ)_{q}, wherein: A is a 3E10 antibody or antigen-binding fragment thereof, L is a linker, P is an ASO described herein, r is an integer from 1 to 4, and q is an integer from 1 to 16.

Examples of ASOs that can be used in exon skipping methodologies are shown in Tables 6-8.

TABLE 6
Example genes targeted by exon skipping oligos.

| Neurogenetic Diseases | Mutant Protein (Genes) | Exon(s) or intronic |
|---|---|---|
| Ataxia telangiectasia | Ataxia telangiectasia | Exons 8 and 55 |
| | mutated (ATM) | (Siva et al. Nucleic Acid |
| | | Ther. 2014 Feb.; 24(1): 69-86.) |
| Congenital disorder of | phosphomannomutase 2 | Intronic 5′ and 3′ |
| glycosylation | (PMM2) | cryptic splice sites etween |
| | | exon 7 and 8 |
| | | (Siva et al. Nucleic Acid |
| | | Ther. 2014 Feb.; 24(1): 69-86.) |
| Frontotemporal | Microtubule-associated | Exon 10 |
| dementia and | protein tau (MAPT) | (Siva et al. Nucleic Acid |
| Parkinsonism linked | | Ther. 2014 Feb.; 24(1): 69-86.) |
| to chromosome 17 | | |
| Niemann Pick disease | Niemann-Pick C1 (NPC1) | Intronic cryptic 5′ |
| type C | | splice site between exon 9 and 10 |
| | | (Siva et al. Nucleic Acid |
| | | Ther. 2014 Feb.; 24(1): 69-86.) |
| Neurofibromatosis | Neurofibromin (NF1) | Intronic cryptic 5′ |
| type 1 | | splice site between exon 4 |
| | | and 4a; exon 30 and 31; |
| | | exon 45 and 46; and exon 9a and 9b |
| | | (Siva et al. Nucleic Acid |
| | | Ther. 2014 Feb.; 24(1): 69-86.) |
| Neurofibromatosis | Merlin (NF2) | Intronic cryptic 5′ |
| type 2 | | splice site between exon 13 and 14 |
| | | (Siva et al. Nucleic Acid |
| | | Ther. 2014 Feb.; 24(1): 69-86.) |
| Megalencephalic | Oligomeric plasma | Intronic cryptic 5′ |
| leukoencephalopathy | membrane (MLC1) | splice site between exon 10 and 11 |
| with subcortical cysts | | (Silva et al. 2014) |
| type 1 | | |
| Pelizaeus-Merzbacher | Proteolipid protein (PLP1) | Exon 3A/3B |
| disease | | (Silva et al. 2014) |
| Familial dysautonomia | Inhibitor kinase complex- | Exon 20 |
| | associated protein | (U.S. Pat. No. 11,179,472) |
| | (IKBKAP) | |
| Spinal muscular atrophy | Survival of motor neuron | Exon 7 |
| | (SMN2) | (U.S. Pat. No. 11,179,472) |
| Myotonic dystrophy (DM) Type I | Dystrophia myotonica | Exon 15 |
| | protein kinase (DMPK) | (U.S. Pat. No. 11,179,472) |
| Myotonic dystrophy (DM) Type II | Cellular nucleic acid- | Between exon 1 and 2 |
| | binding protein | (U.S. Pat. No. 11,179,472) |
| | (CNBP/ZNF9) | |
| Frontotemporal lobar | TAR DNA-binding protein | Exon 6 |
| degeneration (FTLD), | 43 (TARDBP) | (U.S. Pat. No. 11,179,472) |
| amyotrophic lateral sclerosis | | |
| (ALS) | | |
| Hutchinson-Gilford syndrome | Prelamin A (LMNA) | Exons 4 and 5 |
| | | (U.S. Pat. No. 11,179,472) |
| Fukuyama congenital muscular | Fukutin (FKTN) | Exons 4 and 5 |
| dystrophy (FCMD), Walker- | | (U.S. Pat. No. 11,179,472) |
| Warburg syndrome (WWS), limb- | | |
| girdle muscular dystrophy type | | |
| 2M (LGMD2M), and dilated | | |
| cardiomyopathy type 1X | | |
| (CMD1X) | | |
| Parkinson′s Disease PD | LRRK2 | Exon 41 |
| | | (Korecka et al., Mol Ther |
| | | Nucleic Acids. 2020 Sep. |
| | | 4; 21: 623-635) |
| Cardiovascular Diseases | Mutant Protein (Genes) | Exon(s) or intronic |
| Familial hypercholesterolemia | LDL receptor protein | Exon 12 |
| (FH) | (LDLR) | (U.S. Pat. No. 11,179,472) |
| | APOB | Exon 27 |
| | | (Disterer et al. Mol Therap |
| | | 2013 21(3); 602-609) |
| Metabolic Diseases | Mutant Protein (Genes) | Exon(s) or intronic |
| Medium-chain acyl-coenzyme A | Medium-chain acyl-CoA | Exon 11 |
| dehydrogenase deficiency | dehydrogenase (ACADM) | (Bruun et al. Molecular |
| | | Genetics and Metabolism |
| | | Volume 110, Issues 1-2, |
| | | Pages 122) |
| Phenylketonuria | phenylalanine hydroxylase | Exon 11 |
| | (PAH) | (Heintz et al. Mol Genet |
| | | Metab. 2012 |
| | | Aug.; 105(4): 559-565) |
| Cancers | Mutation Protein (Genes) | Exon(s) or intronic |
| Colorectal cancer, lung cancer | K-Ras (KRAS) | Exon 2 |
| | | (WO/2015/091525) |
| Skeletal Muscle Disorders | Mutation Protein (Genes) | Exon(s) or intronic |
| Duchenne muscular dystrophy | Myostatin (MSTN) | Exon 2 |
| (DMD) | | (Kemaladewi, DU et al. |
| | | BMC Med Genomics 4, 36 |
| | | (2011)) |
| Duchenne muscular dystrophy | Dystrophin (DMD) | Exons 45, 51, 53 |
| (DMD) | | (Kemaladewi, DU et al. |
| | | BMC Med Genomics 4, 36 |
| | | (2011)) |
| Becker muscular dystrophy | Dystrophin (DMD) | Exons 9, 25, 27, 31, 37, 38, |
| | | 41, 72 and 74 |
| | | (Okubo et al. Hum Genet. |
| | | 2020; 139(2): 247-255) |
| Lung Disorders | Mutation Protein (Genes) | Exon(s) or intronic |
| Cystic fibrosis | Cystic fibrosis | Exon 23 |
| | transmembrane | (Kim et al., 2022) |
| | conductance regulator | |
| | (CFTR) | |
| Other diseases | Mutation protein (Genes) | Exon(s) or intronic |
| Irritable Bowel Syndrome | IL23R | Exon 9 |
| | | (U.S. Pat. No. 9,868,776B2) |
| Dystrophic Epidermolysis Bullosa | COL7A1 | Exons 73, 74, and 80 |
| | | (U.S. Pat. No. 9,340,783B2) |

TABLE 7
Example sequences of oligos for use in treating various disorders.

| | | Target | | SEQ |
|---|---|---|---|---|
| Disease | Target Gene | exon | Sequence | ID NO |
| Congenital | PMM2 | 7 | UAGCUGCAAAGCAAGUGAAGCGGAC | 201 |
| disorder of | (NM_000303.3) | | AUCACAAACACAACCUACCUCAGGC | 202 |
| glycosylation | | | | |
| Familial | IKBKAP | 20 | UCGCAAACAGUACAAUGGCG | 203 |
| dysauUonomia | (NM_003640.5) | | AACAGUACAAUGGCG | 204 |
| | | | UCGCAAACAGUACAA | 205 |
| | | | ACUAGUCGCAAACAG | 206 |
| | | | AGCUAACUAGUCGCA | 207 |
| Duchenne | MSTN | 2 | AGCCCAUCUUCUCCUGGUCCUGGGAAGG | 208 |
| Muscular | (NM_005259.3) | | | |
| Dystrophy | DMD | 44 | GUGUCUUUCU GAGCCGCCAU UUCUCA | 209 |
| | (NM_004006.3) | | UCUGUUAGCC ACGCCGCCAU UU | 210 |
| | | | CUGUUAGCCA CUCGCCGCCA UUUC | 211 |
| | | | UUGUGUCUUU CUUCUGUUAG CCAC | 212 |
| | | | UGUUAGCCAC UGAUCUCAAC AGAUCU | 213 |
| | | | GUUCAGCUUC UGUUCUCAAC AGAUCU | 214 |
| | | | UUGUGUCUUU CUGCGCCGCC AUUUCU | 215 |
| | | | CUGAGAAACU GUUCGCCGCC AUUUCU | 216 |
| | | | UCUCAGGAAU UUGCGCCGCC AUUUCU | 217 |
| | | | UGUUAGCCAC UGACGCCGCC AUUUCU | 218 |
| | | | GUUCAGCUUC UGUCGCCGCC AUUUCU | 219 |
| | | | AUUCUCAGGA AUUUGAACGC | 220 |
| | | | CGCCAUUUCU | |
| | | | AUUUGUGUCU UUCUGUUUCU | 221 |
| | | | CAACAGAUCU | |
| | | | AUUCUCAGGA AUUUGUUUCU CAACA | 222 |
| | | | AUUUGUGUCU UUCUGAACGC | 223 |
| | | | CGCCAUUUCU | |
| | | | UCUCAGGAAU UUGUCUCAAC AGAUCU | 224 |
| | | 45 | GUUUGCCGCUGCCUCCUGGAGUUCCU | 225 |
| | | | GUUUGCCGCUGCCCUGGAGUUCCU | 226 |
| | | | CAGUUUGCCGCUGCCCAUCCUGGAGUUC | 227 |
| | | | CU | |
| | | | CAGUUUGCCGCUGCCCUGGAGUUCCU | 228 |
| | | | UUCUUCCCCAGUUGCGCCAUCCUGGAGU | 229 |
| | | | UC | |
| | | | CAGACCUCCUGCCACGCCAUCCUGGAGU | 230 |
| | | | UC | |
| | | | UUGCCGCUGCCCACAUCCUGGAGUUC | 231 |
| | | | GCCGCUGCCCACAUCCUGGAGUUCCU | 232 |
| | | | CCGCUGCCCAAUGUCCUGGAGUUCCU | 233 |
| | | | GCUGCCCAAUGCCAUCCUGGAGUUC | 234 |
| | | | ″GCUGCCCAAUGCCAUCCUGGAGUUC | 235 |
| | | | UUGCCGCUGCCCAAUGCCAUCCUGG″ | 236 |
| | | | ACAGUUUGCCGCUGCCCAAUGCCAU | 237 |
| | | | UGACAACAGUUUGCCGCUGCCCAAU | 238 |
| | | | UGUUCUGACAACAGUUUGCCGCUGC | 239 |
| | | | UUCAAUGUUCUGACAACAGUUUGCC | 240 |
| | | | UUGCAUUCAAUGUUCUGACAACAGU | 241 |
| | | | CCCAGUUGCAUUCAAUGUUCUGACA | 242 |
| | | | CCAAUGCCAUCCUGGAGUUCCUGUAA | 243 |
| | | | CAA UGC CAU CCU GGA GUU CCU G | 244 |
| | | | GCC CAA UGC CAU CCU GGA GUU CCU | 245 |
| | | | GUA AGA UAC C | |
| | | | GCC CAA UGC CAU CCU GGA G?U CCU | 246 |
| | | | GUA AGA U | |
| | | | UUG CCG CUG CCC AAU GCC AUC CUG | 247 |
| | | | GAG UUC CUG UAA GAU | |
| | | 50 | CAAUGCCAUCCUGGAGUUCCUG | 248 |
| | | | AUGGGAUCCAGUAUACUUACA | 249 |
| | | | AUGGGAUCCAGUAUACUUAC | 250 |
| | | | UGGGAUCCAGUAUACUUAC | 251 |
| | | | AUGGGAUCCAGUAUACUUA | 252 |
| | | | GAUCCAGUAUACUUACAGG | 253 |
| | | 51 | AAUGGGAUCCAGUAUACUU | 254 |
| | | | CUCCAACAUC AAGGAAGAUG | 255 |
| | | | GCAUUUCUAG | |
| | | | UCAAGGAAGAUGGCAUUUCU | 256 |
| | | | GUGUCACCAGAGUAACAGUCUGAGUAGG | 257 |
| | | | AG | |
| | | | AGGUUGUGUCACCAGAGUAACAGUCUGA | 258 |
| | | | GU | |
| | | | GGCAGUUUCCUUAGUAACCACAGGUUGU | 259 |
| | | | GU | |
| | | | AGAUGGCAGUUUCCUUAGUAACCACAGG | 260 |
| | | | UU | |
| | | | AUGGCAUUUCUAGUUUGGAGAUGGCAGU | 261 |
| | | | UU | |
| | | | CUC CAA CAU CAA GGA AGA UGG CAU | 262 |
| | | | UUC UAG | |
| | | | CUCCAACAUCAAGGAAGAUGGCAUUUCU | 263 |
| | | | AG | |
| | | 53 | CCUCCGGUUC UGAAGGUGUU C | 264 |
| | | | AUU CAA CUG UUG CCU CCG GUU CUG | 265 |
| | | | AAG GUG | |
| | | | CUG AAG GUG UUC UUG UAC UUC AUC C | 266 |
| | | | CAU UCA ACU GUU GCC UCC GGU UCU | 267 |
| | | | GAA GGU G | |
| | | | CCUCCGGUUCUGAAGGUGUUC | 268 |
| | | | GUUGCCUCCGGUUCUGAAGGUGUUC | 269 |
| | | | GUUGCCUCCGGUUCUGAAGGUGUUC | 270 |
| | | | CCUCCGGUUCUGAAGGUGUUC | 271 |
| | | | GUUGCCUCCGGUUCUGAAGGUGUUC | 272 |
| | | 55 | GCAGCCUCUCGCUCACUCACC | 273 |
| | | | CCAAAGCAGCCUCUCGCUCAC | 274 |
| | | | UUCUUCCAAAGCAGCCUCUCG | 275 |
| | | | AUCUAUGAGUUUCUUCCAAAG | 276 |
| | | | UGUUGCAGUAAUCUAUGAGUU | 277 |
| | | | CAGGGGGAACUGUUGCAGUAA | 278 |
| | | | UUUCCAGGUCCAGGGGGAACU | 279 |
| | | | GCAAGAAACUUUUCCAGGUCC | 280 |
| | | | CAG CAG CAG | 281 |
| Myotonic | DMPK | 3′-UUR | CAG CAG CAG CAG | 282 |
| Dystrophy 1 | (NM_004409.5) | | CAG CAG CAG CAG CAG | 283 |
| (DM1) | | | CAG CAG CAG CAG CAG CAG | 284 |
| | | | CAG GCA GGC | 285 |
| Myotonic | ZNF9 | InUron 1 | CAG GCA GGC AGG | 286 |
| Dystrophy 2 | (NM_ | | AGG CAG GCA | 287 |
| (DM2) | 001127192.2) | | AGG CAG GCA GGC | 288 |
| | | | GGC AGG CAG | 289 |
| | | | GGC AGG CAG GCA | 290 |
| | | | UCACUUUCAU AAUGCUGG | 291 |
| Spinal Muscular | SMN2 | 7 | CUGCCAAGGA GGGCCCUGCU CAGCC | 292 |
| Atrophy, type 3 | (NM_017411.4) | | | |
| Bladder cancer | HRAS | 2 | CGCACUCUUG CCCACACCGC CGGCG | 293 |
| | (NM_005343.4) | | CUCACCUCUA UAGUGGGGUC GUAUU | 294 |
| | | | UUGCCCACAC CGCCGGCGCC C | 295 |
| | | | UUGCCCACAC CGACGGCGCC C | 296 |
| | | | UAUAGUGGGG UCGUAUUCGU CCACA | 297 |
| | | | UGGUUCUGGA UCAGCUGGAU GGUCA | 298 |
| | | | CGGGGUCCUC CUACAGGGUC UCCUG | 299 |
| | | | GUCAUCGCUC CUCAGGGGCC UGCGG | 300 |
| | | | CCCACCACCA CCAGCUUAUA UUCCG | 301 |
| | | | GGGUCUCCUG CCCCACCUGC CAAGG | 302 |
| | | | UUCAGUCAUU UUCAGCAGGC CUUAU | 303 |
| Bladder cancer; | KRAS | 2 | UUUACCUCUA UUGUUGGAUC AUAUU | 304 |
| Breast cancer: | | | | |
| Gastric cancer; | (NM_004985.5) | | AUGAAAAUGG UCAGAGAAAC CUUUA | 305 |
| Acute Myeloid | | | | |
| Leukemia; | | | UCUGUAUCAA AGAAUGGUCC UGCAC | 306 |
| Lung cancer; | | | CAGUAAUAUG CAUAUUAAAA CAAGA | 307 |
| Pancreatic | | | AAUAAAAAUA AUGAAAAUGU GACUA | 308 |
| carcinoma; | | | UAUUAGAACA UGUCACACAU AAGGU | 309 |
| RAS-associated | | | AGUUUAUAUU CAGUCAUUUU CAGCA | 310 |
| autoimmune | | | UACGCCACCA GCUCCAACUA CCACA | 311 |
| leukoproliferative | | | GCUGUAUCGU CAAGGCACUC UUGCC | 312 |
| disorder type | | | UCGUCCACAA AAUGAUUCUG AAUUA | 313 |
| IV | | | UUUCACACCA GCAAGAACCU GUUGG | 314 |
| RAS-associated | NRAS | 2 | CCUCACCUCU AUGGUGGGAU CAUAU | 315 |
| autoimmune | (NM_002524.5) | | ACCACCAGUU UGUACUCAGU CAUUU | 316 |
| lympho- | | | CAACACCACC UGCUCCAACC ACCAC | 317 |
| proliferative | | | GUGCGCUUUU CCCAACACCA CCUGC | 318 |
| syndrome | | | UGGUUCUGGA UUAGCUGGAU UGUCA | 319 |
| type IV; | | | GGUGGGAUCA UAUUCAUCUA CAAAG | 320 |
| Colorectal; | | | AAUCAGGGUU AAUUGGCGAG CCACA | 321 |
| Thyroid | | | CAGGAUCAGG UCAGCGGGCU ACCAC | 322 |
| carcinoma, | | | AAA AAU GUC UCC UGC GAG AGC AAA C | 323 |
| follicular | | | | |
| Valosine- | VCP | 5 | CCA CCA UGG ACA AGA AAA AUG UCU C | 324 |
| containing | (NM_007126.5) | | CAG CAC GCA UCC CAC CAU GGA C | 325 |
| protein (VCP)- | | | AUC ACU GUG UCU GGA GCA ACA AUG C | 326 |
| associated | | | UCG UUU GAU AGG CUC CCC UUC GCA G | 327 |
| inclusion body | | | AUC AGG GAG AAA ACU CAC CUC UCG U | 328 |
| myopathy (IBM) | | | ACCUACCCAG UUCGGAAUGA UC | 329 |
| Inflammatory | IL23R | 9 | CUACCCAGUU CGGAAUGA | 330 |
| Bowel diseases | (NM_144701.3) | | UACCCAGUUC GGAAU | 331 |
| (IBS) | | | AUGUCUCCUC UGUUGUCUAA UG | 332 |
| | | | UAACAGCAAA GACGAUCAUU CC | 333 |
| | | | GGGCUCUGGGCUCCUGAGCC | 334 |
| Hutchison- | LMNA | 11-12 | CCGAGCUGCUGCAGUGGGAG | 335 |
| Gilford Progeria | (NM_ | | GCAGGUCCCGCACAGCACGG | 336 |
| Syndrome | 001406990.1) | | UGGCAGAUGCCUUGUCGGCA | 337 |
| (HGPS) | | | GAGCCGCUGGCAGAUGCCUU | 338 |
| | | | CUGAGCCGCUGGCAGAUGCC | 339 |
| | | | AGGAGAUGGGUCCGCCCACC | 340 |
| | | | CAGAGGAGAUGGGUCCGCCC | 341 |
| | | | GCCAGAGGAGAUGGGUCCGC | 342 |
| | | | AGGUCCCGGGAGAAGGGA | 343 |
| Alagille | JAG1 | 13 | CAGCCAGGUCCCGGGAGA | 344 |
| syndrome | (NM_000214.3) | | GGAGACAGCCAGGUCCCG | 345 |
| | | | AGUCUGGAGACAGCCAGG | 346 |
| | | | GAGCAAGUCUGGAGACAG | 347 |
| | | | AAAAGGAGCAAGUCUGGA | 348 |
| | | | GGGACAAAAGGAGCAAGU | 349 |
| | | | GCAGUGGUAGUAAGUGGG | 350 |
| | | | GGGAUCAGAUCACAGCCA | 351 |
| | | | CAGAUCACAGCCAUGCAC | 352 |
| | | | CCCAGCGUCGCCCUGGGC | 353 |
| Uuberous | TSC2 | 4 | CCCAUCCCAGCGUCGCCC | 354 |
| Sclerosis | (NM_000548.5) | | CGUCACCCAUCCCAGCGU | 355 |
| Complex | | | CCUGACGUCACCCAUCCC | 356 |
| | | | GGCAGCCUGACGUCACCC | 357 |
| | | | CAGUGGGCAGCCUGACGU | 358 |
| | | | ACAGUCAGUGGGCAGCCU | 359 |
| | | | ACAGGACAGUCAGUGGGC | 360 |
| | | | ACAGGAUCAGCAGAGCCU | 361 |
| | | | AUCAGCAGAGCCUGCCAG | 362 |
| | | | AGGGCUUAAA AUUUCUCCUG C | 363 |
| Usher Syndrome | USH2A | 62 | CCAAGGGCUU AAAAUUUCUC C | 364 |
| | (NM_206933.4) | | UUUGAAUCAG AGUCCAAGGG C | 365 |
| | | | AAGUUUCUCA GUCCACUUGG G | 366 |
| | | | CCGGCCAUUC UCUUUCUGUU C | 367 |
| | | | AUGCCCGGCC AUUCUCUUUC | 368 |
| | | | UGACCACUGU AGUAGCAAUG | 369 |
| | | | ACACCAUUGG UUCUCAUAGG | 370 |
| | | | AUGGUUUGCA GCCACAACAC | 371 |
| | | | CCACAACACC AAUGCGAUAU | 372 |
| | | | GCAUGGUUUG CAGCCACAAC | 373 |
| | | | CUUGGGGAAG AUUCUAAGGU | 374 |
| | | | GUAUCCGCUG AGGGAAUA | 375 |
| Aicardi- | ADAR | 2 or 3 | GUGUAGUAUC CGCUGAGG | 376 |
| Goutieres | (NM_001193495) | | GAUGGGUGUA GUAUCCGC | 377 |
| syndrome-6 | | | AAAUGGAUGG GUGUAGUA | 378 |
| (AGS6) | | | CCUUGAAAUG GAUGGGUG | 379 |
| | | | GGCGCAGAAC CACCAGGU | 380 |
| Familial | ATP1A2 | 22 or 23 | AGGGAGUUGC AAUAUCGA | 381 |
| hemiplegic | (NM_000702) | | | |
| migraine-2; | | | | |
| Familiar Basilar | | | | |
| Migraine; | | | | |
| Alternating | | | | |
| hemiplegia of | | | | |
| childhood | | | | |
| Progressive | PRICKLE2 | 4 or 5 | GCCUCCACAG AUUCAGGG | 382 |
| Myoclonic | (NM_198859) | | | |
| Epilepsy 5 | | | | |
| Mental | SETD5 | 4 or 5 | AAACUUCAUU CUGGAACA | 383 |
| Retardation-23 | (NM_001080517) | | | |
| (MRD23); | | | | |
| 3p25 | | | | |
| Microdeletion | | | | |
| Syndrome | | | | |
| Leuko- | EIF2B5 | 12 or 13 | AGGGCUUUGG CCAUUUUC | 384 |
| encephalopathy | (NM_003907) | | | |
| with | EIF2B1 | 6 | ACAGUUUGCA CUUCCGGG | 385 |
| vanishing white | (NM_001414) | | | |
| matter | EIF2B2 | 1 | CGCCUAAGGA AUUCACUC | 386 |
| | (NM_014239) | | | |
| Zellweger | PEX1 | 10, 19, | CGCCUAAGGA AUUCACUC | 387 |
| Syndrome; | (NM 000466) | 21, | | |
| Heimler | | or 14 | | |
| Syndrome-1 | PEX1 | 10, 18, | GGAUACGCCU AAGGAAUU | 388 |
| | (NM_001282677) | 20, | | |
| | | or 13 | | |
| | PEX1 | 10, 19, | GCCAGUGCCC AUAGCGGG | 389 |
| | (NM_001282678) | 21, | | |
| | | or 14 | | |
| Early Infantile | STXBP1 | 18, or | CUUAUGCCAG UGCCCAUA | 390 |
| Epileptic | (NM_003165) | 19 | | |
| encephalopathy- | STXBP1 | 18 | CCCAAGGGAG CCACGCGU | 391 |
| 4 | (NM 001032221) | | | |
| Familial Infantile | PRRT2 | 1 | AAGGGAGCCA CGCGUUAC | 392 |
| convulsion with | (NM_001256443) | | | |
| paroxysmal | PRRT2 | 1 | AGCCCAAGGG AGCCACGC | 393 |
| choreoathetosis; | (NM_145239) | | | |
| Episodic | PRRT2 | 1 | CUUCCGGGAC AGUGUGGA | 394 |
| kinesigenic | (NM_001256442) | | | |
| dyskenisia 1; | | | | |
| Benign familial | | | | |
| infantile | | | | |
| seizures-2 | | | | |
| Generalized | STX1B | 6, or 7 | UUCUUGGCAG CUGGAACA | 395 |
| epilepsy with | (NM_0582874) | | | |
| febrile seizure | | | | |
| plus type 9 | | | | |
| Smith-Magenis | RAI1 | 4 | GUAGUCCUGG UGGCAUGC | 396 |
| syndrome | (NM 030665) | | | |
| Pitt-Hopkins | TCF4 | 10 | CCCCUGUAGU CCUGGUGG | 397 |
| syndrome | (NM_001243236) | | | |
| | TCF4 | 10 | CUGUAGUCCU GGUGGCAU | 398 |
| | (NM_001243235) | | | |
| | TCF4 | 10 | UGGCAUCACG GACAAUGC | 399 |
| | (NM_001243234) | | | |
| Niemann-Pick | NPC1 | 15 | CGACAAAGAG AUUCAGCA | 400 |
| disease type C1 | (NM_000271) | | | |
| Episodic Ataxia | CACNA1A | 36, or | GACGGCGACA AAGAGAUU | 401 |
| type 2; | (NM_000068) | 37 | | |
| Familial | CACNA1A | 36, or | AUGAUGACGG CGACAAAG | 402 |
| Hemiplagic | (NM_023035) | 37 | | |
| Migraine; | CACNA1A | 36, or | UGUCCAUGAU GACGGCGA | 403 |
| SpinoCerebellar | (NM_001174080) | 37 | | |
| Ataxia type 6 | CACNA1A | 36, or | AAAGUUGUCC AUGAUGAC | 404 |
| | (NM_001127222) | 37 | | |
| | CACNA1A | 36, or | GACUUGUGGG UGUUCUCA | 405 |
| | (NM_001127221) | 37 | | |
| Hereditary | DNMT1 | 29, or | GAGUGGACUU GUGGGUGU | 406 |
| Sensory | (NM_001379) | 14 | | |
| Neuropathy type | DNMT1 | 30, or | CGCUGGAGUG GACUUGUG | 407 |
| IE; | (NM_001130823) | 15 | | |
| Autosomal | DNMT1 | 29, or | UAGCUCGCUG GAGUGGAC | 408 |
| dominant | (NM_001318730) | 14 | | |
| cerebellar ataxia, | DNMT1 | 30, or | GUCUUCUGCC UUGGGCUU | 409 |
| deafness and | (NM_001318731) | 15 | | |
| narcolepsy | | | | |
| Neurofibromatosis | NF2 | 14 | GAUAGGUCUU CUGCCUUG | 410 |
| type 2; | (NM_181830) | | | |
| NF2-related | NF2 | 15 | AUCUAAAAUC UGCUUCUU | 411 |
| meningioma; | (NM_181829) | | | |
| Shwannomatosis | NF2 | 4 | AUGCAGAUAG GUCUUCUG | 412 |
| 1 | (NM_181833) | | | |
| | NF2 | 16 | GUCUUCUGCC UUGGGCUU | 413 |
| | (NM_181832) | | | |
| | NF2 | 15 | CUCAAAUGCA GAUAGGUC | 414 |
| | (NM_181828) | | | |
| | NF2 | 16 | UCUUUUCCAU GUAUUCCA | 415 |
| | (NM_016418) | | | |
| | NF2 | 15 | ACCACGUUCA CCCCGUUC | 416 |
| | (NM_000268) | | | |
| Phelan- | SHANK3 | 16 | GGCCGGUCAG GAGGGCGG | 417 |
| McDermid | (NM_033517) | | | |
| syndrome | | | | |
| (PHMDS); | | | | |
| Schizophrenia- | | | | |
| 15 (SCZD15)″ | | | | |
| Metachromatic | ARSA | 2, or 3 | GAGCCGGCCG GUCAGGAG | 418 |
| Leukodystrophy | (NM_000487) | | | |
| | ARSA | 3, or 4 | ACCGGGAGCC GGCCGGUC | 419 |
| | (NM_001085425) | | | |
| | ARSA | 3, or 4 | UCCGAACCGG GAGCCGGC | 420 |
| | (NM_001085426) | | | |
| | ARSA | 3, or 4 | UCCGAACCGG GAGCCGGC | 421 |
| | (NM_001085427) | | | |
| | ARSA | 2, or 3 | CCAGAAAAAAUAAAUACUUUC | 422 |
| | (NM_001085428) | | | |
| Cystic Fibrosis | CTFR | 24 | AAUGUUCCAGAAAAAAUA | 423 |
| | (EF466097.1) | | UCUAAAUGUUCCAGAAAA | 424 |
| | | | CCACUGUUCAUAGGGAUC | 425 |
| | | | CCAUACCUGUAUAGGAGAGAUUCAUA | 426 |
| Familial | APOB | 27 | CCAUACCUGUAUAG GAGAGAAU-UAGA | 427 |
| Hyper- | (NM_000384.3) | 27 | CCAUACCUGUAUAGGAGAGAAUUAGAUU | 428 |
| cholesterolemia | | | CAUA | |
| | | 27 | AUCCUCUUUGGUAACCUCACCUCAC | 429 |
| Phenylketonuria | PAH | 11 | UCUCCACGGUCGCCCUUCAGCCCGCGUU | 430 |
| | (NM_ | | CU | |
| | 001354304.2) | | | |
| Dystrophic | COL7A1 | 73, 74, | UCUCCACGGUCGCCCUUCAGCCCGC | 431 |
| Epidermolysis | (NM_000094.4) | or 80 | CCUUUCUCUCCCCGUUCUCCCUGAA | 432 |
| Bullosa, | | | CCUUUCUCUCCCCAUUCUCCCUGAA | 433 |
| autosomal | | | CCACCUGUUCUCCACGUUCUCCUUUC | 434 |
| dominant | | | GGCCUCUUGGACCCUGCAGACCCU | 435 |
| | | | GGCCUCUUGGACCCUACAGACCCU | 436 |
| | | | ACCUACCCAG UUCGGAAUGA UC | 437 |
| Inflammatory | ILR23 | 9 | CUACCCAGUU CGGAAUGA | 438 |
| Bowel Disease | (NM_144701.3) | | UACCCAGUUC GGAAU | 439 |
| | | | AUGUCUCCUC UGUUGUCUAA UG | 440 |
| | | | UAACAGCAAA GACGAUCAUU CC | 441 |
| | | | GAAACAACUUUUUGUUUGAGAA | 442 |
| | | | GUUUUUCCAGAGUGCACAACUA | 443 |
| | | | F: AGUCCAAGUGUGGCUCAAA | 444 |
| | | | R: UUUGAGCCACACUUGGACU | 445 |
| Frontotemporal | MAPT | 10 | F: GGCUCAAAGGAUAAUAUCA | 446 |
| dementia with | (NM_ | | R: UGAUAUUAUCCUUUGAGCC | 447 |
| parkinsonism | 001377265.1) | | F: GCAACGUCCAGUCCAAGUG | 448 |
| linked to | | | R: CACUUGGACUGGACGUUGC | 449 |
| chromosome 17 | | | AGACAGACCUGAUCACCUACCUGGU | 450 |
| Parkinson's | LRRK2 | 41 | GGUAUCUGCCAGAAAAUGCACAGGA | 451 |
| Disease | (NM_198578.4) | | CUACCAGCCUACCAUGUUACCUUGA | 452 |

TABLE 8
Example single stranded oligonucleotide sequences capable of inducing exon skipping
for use in treating DMD.

| | Target | Target | | |
|---|---|---|---|---|
| Disease | Gene | exon | Sequence | SEQ ID NO |
| Duchenne Muscular | DMD | 43 | CCACAGGCGUUGCACUUUGCAAUGC | 453 |
| Dystrophy | | | CACAGGCGUUGCACUUUGCAAUGCU | 454 |
| | | | ACAGGCGUUGCACUUUGCAAUGCUG | 455 |
| | | | CAGGCGUUGCACUUUGCAAUGCUGC | 456 |
| | | | CAGGCGUUGCACUUUGCAAUGCUGC | 457 |
| | | | AGGCGUUGCACUUUGCAAUGCUGCU | 458 |
| | | | GGCGUUGCACUUUGCAAUGCUGCUG | 459 |
| | | | GCGUUGCACUUUGCAAUGCUGCUGU | 460 |
| | | | CGUUGCACUUUGCAAUGCUGCUGUC | 461 |
| | | | CGUUGCACUUUGCAAUGCUGCUG | 462 |
| | | | GUUGCACUUUGCAAUGCUGCUGUCU | 463 |
| | | | UUGCACUUUGCAAUGCUGCUGUCUU | 464 |
| | | | UGCACUUUGCAAUGCUGCUGUCUUC | 465 |
| | | | GCACUUUGCAAUGCUGCUGUCUUCU | 466 |
| | | | CACUUUGCAAUGCUGCUGUCUUCUU | 467 |
| | | | ACUUUGCAAUGCUGCUGUCUUCUUG | 468 |
| | | | CUUUGCAAUGCUGCUGUCUUCUUGC | 469 |
| | | | UUUGCAAUGCUGCUGUCUUCUUGCU | 470 |
| | | | UUGCAAUGCUGCUGUCUUCUUGCUA | 471 |
| | | | UGCAAUGCUGCUGUCUUCUUGCUAU | 472 |
| | | | GCAAUGCUGCUGUCUUCUUGCUAUG | 473 |
| | | | CAAUGCUGCUGUCUUCUUGCUAUGA | 474 |
| | | | AAUGCUGCUGUCUUCUUGCUAUGAA | 475 |
| | | | AUGCUGCUGUCUUCUUGCUAUGAAU | 476 |
| | | | UGCUGCUGUCUUCUUGCUAUGAAUA | 477 |
| | | | GCUGCUGUCUUCUUGCUAUGAAUAA | 478 |
| | | | CUGCUGUCUUCUUGCUAUGAAUAAU | 479 |
| | | | UGCUGUCUUCUUGCUAUGAAUAAUG | 480 |
| | | | GCUGUCUUCUUGCUAUGAAUAAUGU | 481 |
| | | | CUGUCUUCUUGCUAUGAAUAAUGUC | 482 |
| | | | UGUCUUCUUGCUAUGAAUAAUGUCA | 483 |
| | | | GUCUUCUUGCUAUGAAUAAUGUCAA | 484 |
| | | | UCUUCUUGCUAUGAAUAAUGUCAAU | 485 |
| | | | CUUCUUGCUAUGAAUAAUGUCAAUC | 486 |
| | | | UUCUUGCUAUGAAUAAUGUCAAUCC | 487 |
| | | | UCUUGCUAUGAAUAAUGUCAAUCCG | 488 |
| | | | CUUGCUAUGAAUAAUGUCAAUCCGA | 489 |
| | | | UUGCUAUGAAUAAUGUCAAUCCGAC | 490 |
| | | | UGCUAUGAAUAAUGUCAAUCCGACC | 491 |
| | | | GCUAUGAAUAAUGUCAAUCCGACCU | 492 |
| | | | CUAUGAAUAAUGUCAAUCCGACCUG | 493 |
| | | | UAUGAAUAAUGUCAAUCCGACCUGA | 494 |
| | | | AUGAAUAAUGUCAAUCCGACCUGAG | 495 |
| | | | UGAAUAAUGUCAAUCCGACCUGAGC | 496 |
| | | | GAAUAAUGUCAAUCCGACCUGAGCU | 497 |
| | | | AAUAAUGUCAAUCCGACCUGAGCUU | 498 |
| | | | AUAAUGUCAAUCCGACCUGAGCUUU | 499 |
| | | | UAAUGUCAAUCCGACCUGAGCUUUG | 500 |
| | | | AAUGUCAAUCCGACCUGAGCUUUGU | 501 |
| | | | AUGUCAAUCCGACCUGAGCUUUGUU | 502 |
| | | | UGUCAAUCCGACCUGAGCUUUGUUG | 503 |
| | | | GUCAAUCCGACCUGAGCUUUGUUGU | 504 |
| | | | UCAAUCCGACCUGAGCUUUGUUGUA | 505 |
| | | | CAAUCCGACCUGAGCUUUGUUGUAG | 506 |
| | | | AAUCCGACCUGAGCUUUGUUGUAGA | 507 |
| | | | AUCCGACCUGAGCUUUGUUGUAGAC | 508 |
| | | | UCCGACCUGAGCUUUGUUGUAGACU | 509 |
| | | | CCGACCUGAGCUUUGUUGUAGACUA | 510 |
| | | | CGACCUGAGCUUUGUUGUAG | 511 |
| | | | CGACCUGAGCUUUGUUGUAGACUAU | 512 |
| | | | GACCUGAGCUUUGUUGUAGACUAUC | 513 |
| | | | ACCUGAGCUUUGUUGUAGACUAUCA | 514 |
| | | | CCUGAGCUUUGUUGUAGACUAUC | 515 |
| | | | GCAAUGCUGCUGUCUUCUUGCU | 516 |
| | | | AACAAAAUGUACAAGGACCGAC | 517 |
| | | | UGCAAAGUGCAACGCCUGUGGA | 518 |
| | | | AUAGUCUACAACAAAGCUCAGG | 519 |
| | | | CUGUUUUAAAAUUUUUAUAUUA | 520 |
| | | | AUAUAUGUGUUACCUACCCUUG | 521 |
| | | | ACAUUUUGUUAACUUUUUCCCA | 522 |
| | | | CUUUUUCCCAUUGGAAAUCAAG | 523 |
| | | | UUUUUCCCAUUGGAAAUCAAGC | 524 |
| | | | UCCUGUAGCUUCACCCUUUCCA | 525 |
| | | | AAUGUACAAGGACCGACAAGGG | 526 |
| | | | ACAAAAUGUACAAGGACCGACA | 527 |
| | | | AACAAAAUGUACAAGGACCGAC | 528 |
| | | | GGAAAAAGUUAACAAAAUGUAC | 529 |
| | | | UCUCUCCCAGCUUGAUUUCCAA | 530 |
| | | | CUCUCUCCCAGCUUGAUUUCCA | 531 |
| | | | GCCUGUGGAAAGGGUGAAGCUA | 532 |
| | | | GCAAAGUGCAACGCCUGUGGAA | 533 |
| | | | UGCAAAGUGCAACGCCUGUGGA | 534 |
| | | | AGCAUUGCAAAGUGCAACGCCU | 535 |
| | | | AUAGUCUACAACAAAGCUCAGG | 536 |
| | | | AAAGAUAGUCUACAACAAAGCU | 537 |
| | | 44 | GUGGCUAACAGAAGCU | 538 |
| | | | GGGAACAUGCUAAAUAC | 539 |
| | | | AGACACAAAUUCCUGAGA | \|540 |
| | | | CUGUUGAGAAA | 541 |
| | | | AUUUAGCAUGUUCCCAAUUCUC | 542 |
| | | | AAUCGCCUGCAGGUAAAAGCAU | 543 |
| | | | UCUCAGAAAGACACAAAUUCCU | 544 |
| | | | UAUUUAGCAUGUUCCCAAUUCU | 545 |
| | | | AACAGAUCUGUCAAAUCGCCUG | 546 |
| | | | AAUCGCCUGCAGGUAAAAGCAU | 547 |
| | | | UGCUAAAUACAAAUGGUAUCUU | 548 |
| | | | AUUGGGAACAUGCUAAAUACAA | 549 |
| | | | AAAGACACAAAUUCCUGAGAAU | 550 |
| | | | GAAAGACACAAAUUCCUGAGA | 551 |
| | | | AUGAUAUAAAGAUAUUUAAUCA | 552 |
| | | | UUGACAGAUCUGUUGAGAAAUG | 553 |
| | | | GAUUUGACAGAUCUGUUGAGAA | 554 |
| | | | UUGAUCCAUAUGCUUUUACCUG | 555 |
| | | 45 | UUUGCCGCUGCCCAAUGCCAUCCUG | 556 |
| | | | UUUGCCGCUGCCCAAUGCCAUCCUG | 557 |
| | | | UUUGCCICUGCCCAAUGCCAUCCUG | 558 |
| | | | UAGAAUACUGGCAUCUGUUUUU | 559 |
| | | | UGGCAUCUGUUUUUGAGGAUUG | 560 |
| | | | UUGCCGCUGCCCAAUGCCAUCC | 561 |
| | | | GAGGUAGGGCGACAGAUCUAAU | 562 |
| | | | UCUACAGGAAAAAUUGGGAAGC | 563 |
| | | | GCGGCAAACUGUUGUCAGAACA | 564 |
| | | | UUUUGGUAUCUUACAGGAACUC | 565 |
| | | | AGACCUCCUGCCACCGCAGAUU | 566 |
| | | | UCCCAAUUUUUCCUGUAGAAUA | 567 |
| | | | UAGAAUACUGGCAUCUGUUUUU | 568 |
| | | | UUUGCCGCUGCCCAAUGCCAUC | 569 |
| | | | GGAGUUCCUGUAAGAUACCAAA | 570 |
| | | | AGAGGUAGGGCGACAGAUCUAA | 571 |
| | | | CUGUCAGACAGAAAAAAGAGGU | 572 |
| | | | GCUGUCAGACAGAAAAAAGAGG | 573 |
| | | | AACAGCUGUCAGACAGAAAAAA | 574 |
| | | | AAGCCUGAAUCUGCGGUGGCAG | 575 |
| | | | GGGAAGCCUGAAUCUGCGGUGG | 576 |
| | | | AAUUGGGAAGCCUGAAUCUGCG | 577 |
| | | | AAAAAUUGGGAAGCCUGAAUCU | 578 |
| | | | GCCAGUAUUCUACAGGAAAAAU | 579 |
| | | | UGCCAGUAUUCUACAGGAAAAA | 580 |
| | | | AAAAACAGAUGCCAGUAUUCUA | 581 |
| | | | UGUCAGAACAUUGAAUGCAACU | 582 |
| | | | UUGUCAGAACAUUGAAUGCAAC | 583 |
| | | | GUUGUCAGAACAUUGAAUGCAA | 584 |
| | | | AACUCCAGGAUGGCAUUGGGCA | 585 |
| | | | UACAGGAACUCCAGGAUGGCAU | 586 |
| | | | UUACAGGAACUCCAGGAUGGCA | 587 |
| | | | GGUAUCUUACAGGAACUCCAGG | 588 |
| | | | UUUUGGUAUCUUACAGGAACUC | 589 |
| | | | GUUUUGCCUUUUUGGUAUCUUA | 590 |
| | | | UCUUUUCUCAAAUAAAAAGACA | 591 |
| | | 46 | GCUUUUCUUUUAGUUGCUGCUCUUU | 592 |
| | | | CUUUUCUUUUAGUUGCUGCUCUUUU | 593 |
| | | | UUUUCUUUUAGUUGCUGCUCUUUUC | 594 |
| | | | UUUCUUUUAGUUGCUGCUCUUUUCC | 595 |
| | | | UUCUUUUAGUUGCUGCUCUUUUCCA | 596 |
| | | | UCUUUUAGUUGCUGCUCUUUUCCAG | 597 |
| | | | CUUUUAGUUGCUGCUCUUUUCCAGG | 598 |
| | | | UUUUAGUUGCUGCUCUUUUCCAGGU | 599 |
| | | | UUUAGUUGCUGCUCUUUUCCAGGUU | 600 |
| | | | UUAGUUGCUGCUCUUUUCCAGGUUC | 601 |
| | | | UAGUUGCUGCUCUUUUCCAGGUUCA | 602 |
| | | | AGUUGCUGCUCUUUUCCAGGUUCAA | 603 |
| | | | GUUGCUGCUCUUUUCCAGGUUCAAG | 604 |
| | | | UUGCUGCUCUUUUCCAGGUUCAAGU | 605 |
| | | | UGCUGCUCUUUUCCAGGUUCAAGUG | 606 |
| | | | GCUGCUCUUUUCCAGGUUCAAGUGG | 607 |
| | | | CUGCUCUUUUCCAGGUUCAAGUGGG | 608 |
| | | | UGCUCUUUUCCAGGUUCAAGUGGGA | 609 |
| | | | GCUCUUUUCCAGGUUCAAGUGGGAC | 610 |
| | | | CUCUUUUCCAGGUUCAAGUGGGAUA | 611 |
| | | | UCUUUUCCAGGUUCAAGUGGGAUAC | 612 |
| | | | UCUUUUCCAGGUUCAAGUGG | 613 |
| | | | CUUUUCCAGGUUCAAGUGGGAUACU | 614 |
| | | | UUUUCCAGGUUCAAGUGGGAUACUA | 615 |
| | | | UUUCCAGGUUCAAGUGGGAUACUAG | 616 |
| | | | UUCCAGGUUCAAGUGGGAUACUAGC | 617 |
| | | | UCCAGGUUCAAGUGGGAUACUAGCA | 618 |
| | | | CCAGGUUCAAGUGGGAUACUAGCAA | 619 |
| | | | CAGGUUCAAGUGGGAUACUAGCAAU | 620 |
| | | | AGGUUCAAGUGGGAUACUAGCAAUG | 621 |
| | | | GGUUCAAGUGGGAUACUAGCAAUGU | 622 |
| | | | GUUCAAGUGGGAUACUAGCAAUGUU | 623 |
| | | | UUCAAGUGGGAUACUAGCAAUGUUA | 624 |
| | | | UCAAGUGGGAUACUAGCAAUGUUAU | 625 |
| | | | CAAGUGGGAUACUAGCAAUGUUAUC | 626 |
| | | | AAGUGGGAUACUAGCAAUGUUAUCU | 627 |
| | | | AGUGGGAUACUAGCAAUGUUAUCUG | 628 |
| | | | GUGGGAUACUAGCAAUGUUAUCUGC | 629 |
| | | | UGGGAUACUAGCAAUGUUAUCUGCU | 630 |
| | | | GGGAUACUAGCAAUGUUAUCUGCUU | 631 |
| | | | GGAUACUAGCAAUGUUAUCUGCUUC | 632 |
| | | | GAUACUAGCAAUGUUAUCUGCUUCC | 633 |
| | | | AUACUAGCAAUGUUAUCUGCUUCCU | 634 |
| | | | UACUAGCAAUGUUAUCUGCUUCCUC | 635 |
| | | | ACUAGCAAUGUUAUCUGCUUCCUCC | 636 |
| | | | CUAGCAAUGUUAUCUGCUUCCUCCA | 637 |
| | | | UAGCAAUGUUAUCUGCUUCCUCCAA | 638 |
| | | | AGCAAUGUUAUCUGCUUCCUCCAAC | 639 |
| | | | GCAAUGUUAUCUGCUUCCUCCAACC | 640 |
| | | | CAAUGUUAUCUGCUUCCUCCAACCA | 641 |
| | | | AAUGUUAUCUGCUUCCUCCAACCAU | 642 |
| | | | AUGUUAUCUGCUUCCUCCAACCAUA | 643 |
| | | | UGUUAUCUGCUUCCUCCAACCAUAA | 644 |
| | | 50 | CCAAUAGUGGUCAGUCCAGGAGCUA | 645 |
| | | | CAAUAGUGGUCAGUCCAGGAGCUAG | 646 |
| | | | AAUAGUGGUCAGUCCAGGAGCUAGG | 647 |
| | | | AUAGUGGUCAGUCCAGGAGCUAGGU | 648 |
| | | | AUAGUGGUCAGUCCAGGAGCU | 649 |
| | | | UAGUGGUCAGUCCAGGAGCUAGGUC | 650 |
| | | | AGUGGUCAGUCCAGGAGCUAGGUCA | 651 |
| | | | GUGGUCAGUCCAGGAGCUAGGUCAG | 652 |
| | | | UGGUCAGUCCAGGAGCUAGGUCAGG | 653 |
| | | | GGUCAGUCCAGGAGCUAGGUCAGGC | 654 |
| | | | GUCAGUCCAGGAGCUAGGUCAGGCU | 655 |
| | | | UCAGUCCAGGAGCUAGGUCAGGCUG | 656 |
| | | | CAGUCCAGGAGCUAGGUCAGGCUGC | 657 |
| | | | AGUCCAGGAGCUAGGUCAGGCUGCU | 658 |
| | | | GUCCAGGAGCUAGGUCAGGCUGCUU | 659 |
| | | | UCCAGGAGCUAGGUCAGGCUGCUUU | 660 |
| | | | CCAGGAGCUAGGUCAGGCUGCUUUG | 661 |
| | | | CAGGAGCUAGGUCAGGCUGCUUUGC | 662 |
| | | | AGGAGCUAGGUCAGGCUGCUUUGCC | 663 |
| | | | GGAGCUAGGUCAGGCUGCUUUGCCC | 664 |
| | | | GAGCUAGGUCAGGCUGCUUUGCCCU | 665 |
| | | | AGCUAGGUCAGGCUGCUUUGCCCUC | 666 |
| | | | GCUAGGUCAGGCUGCUUUGCCCUCA | 667 |
| | | | CUAGGUCAGGCUGCUUUGCCCUCAG | 668 |
| | | | UAGGUCAGGCUGCUUUGCCCUCAGC | 669 |
| | | | AGGUCAGGCUGCUUUGCCCUCAGCU | 670 |
| | | | GGUCAGGCUGCUUUGCCCUCAGCUC | 671 |
| | | | GUCAGGCUGCUUUGCCCUCAGCUCU | 672 |
| | | | UCAGGCUGCUUUGCCCUCAGCUCUU | 673 |
| | | | CAGGCUGCUUUGCCCUCAGCUCUUG | 674 |
| | | | AGGCUGCUUUGCCCUCAGCUCUUGA | 675 |
| | | | GGCUGCUUUGCCCUCAGCUCUUGAA | 676 |
| | | | GCUGCUUUGCCCUCAGCUCUUGAAG | 677 |
| | | | CUGCUUUGCCCUCAGCUCUUGAAGU | 678 |
| | | | UGCUUUGCCCUCAGCUCUUGAAGUA | 679 |
| | | | GCUUUGCCCUCAGCUCUUGAAGUAA | 680 |
| | | | CUUUGCCCUCAGCUCUUGAAGUAAA | 681 |
| | | | UUUGCCCUCAGCUCUUGAAGUAAAC | 682 |
| | | | UUGCCCUCAGCUCUUGAAGUAAACG | 683 |
| | | | UGCCCUCAGCUCUUGAAGUAAACGG | 684 |
| | | | GCCCUCAGCUCUUGAAGUAAACGGU | 685 |
| | | | CCCUCAGCUCUUGAAGUAAACGGUU | 686 |
| | | | CCUCAGCUCUUGAAGUAAAC | 687 |
| | | | CCUCAGCUCUUGAAGUAAACG | 688 |
| | | | CUCAGCUCUUGAAGUAAACG | 689 |
| | | | CCUCAGCUCUUGAAGUAAACGGUUU | 690 |
| | | | CUCAGCUCUUGAAGUAAACGGUUUA | 691 |
| | | | UCAGCUCUUGAAGUAAACGGUUUAC | 692 |
| | | | CAGCUCUUGAAGUAAACGGUUUACC | 693 |
| | | | AGCUCUUGAAGUAAACGGUUUACCG | 694 |
| | | | GCUCUUGAAGUAAACGGUUUACCGC | 695 |
| | | | CUCUUGAAGUAAACGGUUUACCGCC | 696 |
| | | | ACUAUUGGAGCCUGUAAGUAUA | 697 |
| | | | AGGAAGUUAGAAGAUCUGAGCU | 698 |
| | | | AGAAUGGGAUCCAGUAUACUUA | 699 |
| | | | AGUAUACUUACAGGCUCCAAUA | 700 |
| | | | CAGGCUCCAAUAGUGGUCAGUC | 701 |
| | | | CAAUAGUGGUCAGUCCAGGAGC | 702 |
| | | | GUGGUCAGUCCAGGAGCUAGGU | 703 |
| | | | UUGCCCUCAGCUCUUGAAGUAA | 704 |
| | | | AGUAUACUGGAUCCCAUUCUCU | 705 |
| | | | ACUAUUGGAGCCUGUAAGUAUA | 706 |
| | | | CUAGCUCCUGGACUGACCACUA | 707 |
| | | | GCAAAGCAGCCUGACCUAGCUC | 708 |
| | | | AAACCGUUUACUUCAAGAGCUG | 709 |
| | | | UAAACCGUUUACUUCAAGAGCU | 710 |
| | | | AGAUCUGAGCUCUGAGUGGAAG | 711 |
| | | | AGAAGAUCUGAGCUCUGAGUGG | 712 |
| | | | AGUUAGAAGAUCUGAGCUCUGA | 713 |
| | | | AUGUGUAUGCUUUUCUGUUAAA | 714 |
| | | | AUGGGAUCCAGUAUACUUACA | 715 |
| | | | AUGGGAUCCAGUAUACUUAC | 716 |
| | | | UGGGAUCCAGUAUACUUAC | 717 |
| | | | AUGGGAUCCAGUAUACUUA | 718 |
| | | | GAUCCAGUAUACUUACAGG | 719 |
| | | | GGAUCCAGUAUACUUACAG | 720 |
| | | | AAUGGGAUCCAGUAUACUU | 721 |
| | | 51 | GUACCUCCAACAUCAAGGAAGAUGG | 722 |
| | | | UACCUCCAACAUCAAGGAAGAUGGC | 723 |
| | | | ACCUCCAACAUCAAGGAAGAUGGCA | 724 |
| | | | CCUCCAACAUCAAGGAAGAUGGCAU | 725 |
| | | | CUCCAACAUCAAGGAAGAUGGCAUU | 726 |
| | | | UCCAACAUCAAGGAAGAUGGCAUUU | 727 |
| | | | CCAACAUCAAGGAAGAUGGCAUUUC | 728 |
| | | | CAACAUCAAGGAAGAUGGCAUUUCU | 729 |
| | | | AACAUCAAGGAAGAUGGCAUUUCUA | 730 |
| | | | ACAUCAAGGAAGAUGGCAUUUCUAG | 731 |
| | | | CAUCAAGGAAGAUGGCAUUUCUAGU | 732 |
| | | | AUCAAGGAAGAUGGCAUUUCUAGUU | 733 |
| | | | UCAAGGAAGAUGGCAUUUCUAGUUU | 734 |
| | | | CAAGGAAGAUGGCAUUUCUAGUUUG | 735 |
| | | | AAGGAAGAUGGCAUUUCUAGUUUGG | 736 |
| | | | AGGAAGAUGGCAUUUCUAGUUUGGA | 737 |
| | | | GGAAGAUGGCAUUUCUAGUUUGGAG | 738 |
| | | | GAAGAUGGCAUUUCUAGUUUGGAGA | 739 |
| | | | AAGAUGGCAUUUCUAGUUUGGAGAU | 740 |
| | | | AGAUGGCAUUUCUAGUUUGGAGAUG | 741 |
| | | | GAUGGCAUUUCUAGUUUGGAGAUGG | 742 |
| | | | AUGGCAUUUCUAGUUUGGAGAUGGC | 743 |
| | | | UGGCAUUUCUAGUUUGGAGAUGGCA | 744 |
| | | | GGCAUUUCUAGUUUGGAGAUGGCAG | 745 |
| | | | GCAUUUCUAGUUUGGAGAUGGCAGU | 746 |
| | | | CAUUUCUAGUUUGGAGAUGGCAGUU | 747 |
| | | | AUUUCUAGUUUGGAGAUGGCAGUUU | 748 |
| | | | UUUCUAGUUUGGAGAUGGCAGUUUC | 749 |
| | | | UUCUAGUUUGGAGAUGGCAGUUUCC | 750 |
| | | | UCUAGUUUGGAGAUGGCAGUUUCCU | 751 |
| | | | CUAGUUUGGAGAUGGCAGUUUCCUU | 752 |
| | | | UAGUUUGGAGAUGGCAGUUUCCUUA | 753 |
| | | | AGUUUGGAGAUGGCAGUUUCCUUAG | 754 |
| | | | GUUUGGAGAUGGCAGUUUCCUUAGU | 755 |
| | | | UUUGGAGAUGGCAGUUUCCUUAGUA | 756 |
| | | | UUGGAGAUGGCAGUUUCCUUAGUAA | 757 |
| | | | UGGAGAUGGCAGUUUCCUUAGUAAC | 758 |
| | | | GAGAUGGCAGUUUCCUUAGUAACCA | 759 |
| | | | AGAUGGCAGUUUCCUUAGUAACCAC | 760 |
| | | | GAUGGCAGUUUCCUUAGUAACCACA | 761 |
| | | | AUGGCAGUUUCCUUAGUAACCACAG | 762 |
| | | | UGGCAGUUUCCUUAGUAACCACAGG | 763 |
| | | | GGCAGUUUCCUUAGUAACCACAGGU | 764 |
| | | | GCAGUUUCCUUAGUAACCACAGGUU | 765 |
| | | | CAGUUUCCUUAGUAACCACAGGUUG | 766 |
| | | | AGUUUCCUUAGUAACCACAGGUUGU | 767 |
| | | | GUUUCCUUAGUAACCACAGGUUGUG | 768 |
| | | | UUUCCUUAGUAACCACAGGUUGUGU | 769 |
| | | | UUCCUUAGUAACCACAGGUUGUGUC | 770 |
| | | | UCCUUAGUAACCACAGGUUGUGUCA | 771 |
| | | | CCUUAGUAACCACAGGUUGUGUCAC | 772 |
| | | | CUUAGUAACCACAGGUUGUGUCACC | 773 |
| | | | UUAGUAACCACAGGUUGUGUCACCA | 774 |
| | | | UAGUAACCACAGGUUGUGUCACCAG | 775 |
| | | | AGUAACCACAGGUUGUGUCACCAGA | 776 |
| | | | GUAACCACAGGUUGUGUCACCAGAG | 777 |
| | | | UAACCACAGGUUGUGUCACCAGAGU | 778 |
| | | | AACCACAGGUUGUGUCACCAGAGUA | 779 |
| | | | ACCACAGGUUGUGUCACCAGAGUAA | 780 |
| | | | CCACAGGUUGUGUCACCAGAGUAAC | 781 |
| | | | CACAGGUUGUGUCACCAGAGUAACA | 782 |
| | | | ACAGGUUGUGUCACCAGAGUAACAG | 783 |
| | | | CAGGUUGUGUCACCAGAGUAACAGU | 784 |
| | | | AGGUUGUGUCACCAGAGUAACAGUC | 785 |
| | | | GGUUGUGUCACCAGAGUAACAGUCU | 786 |
| | | | GUUGUGUCACCAGAGUAACAGUCUG | 787 |
| | | | UUGUGUCACCAGAGUAACAGUCUGA | 788 |
| | | | UGUGUCACCAGAGUAACAGUCUGAG | 789 |
| | | | GUGUCACCAGAGUAACAGUCUGAGU | 790 |
| | | | UGUCACCAGAGUAACAGUCUGAGUA | 791 |
| | | | GUCACCAGAGUAACAGUCUGAGUAG | 792 |
| | | | UCACCAGAGUAACAGUCUGAGUAGG | 793 |
| | | | CACCAGAGUAACAGUCUGAGUAGGA | 794 |
| | | | ACCAGAGUAACAGUCUGAGUAGGAG | 795 |
| | | | UAGUAACCACAGGUUGUGUCAC | 796 |
| | | | GUUGUGUCACCAGAGUAACAGU | 797 |
| | | | UCUGAGUAGGAGCUAAAAUAUU | 798 |
| | | | GAGGGUGAUGGUGGGUGACCUU | 799 |
| | | | UAUAAAAUCACAGAGGGUGAUG | 800 |
| | | | UUGAUCAAGUUAUAAAAUCACA | 801 |
| | | | UGAUCAUCUCGUUGAUAUCCUC | 802 |
| | | | UUGAUCAAGCAGAGAAAGCCAG | 803 |
| | | | AGUCGGUAAGUUCUGUCCAAGC | 804 |
| | | | GCCCGGUUGAAAUCUGCCAGAG | 805 |
| | | | CAGAGCAGGUACCUCCAACAUC | 806 |
| | | | GGUACCUCCAACAUCAAGGAAG | 807 |
| | | | CAAGGAAGAUGGCAUUUCUAGU | 808 |
| | | | AGAUGGCAUUUCUAGUUUGGAG | 809 |
| | | | AUGGCAGUUUCCUUAGUAACCA | 810 |
| | | | GUCACCAGAGUAACAGUCUGAG | 811 |
| | | | UCUGAGUAGGAGCUAAAAUAUU | 812 |
| | | | AAAUAUUUUGGGUUUUUGCAAA | 813 |
| | | | GUAUGAGAAAAAAUGAUAAAAG | 814 |
| | | | CAACGAGAUGAUCAUCAAGCAG | 815 |
| | | | GAGGGUGAUGGUGGGUGACCUU | 816 |
| | | | AUAAAAUCACAGAGGGUGAUGG | 817 |
| | | | UAUAAAAUCACAGAGGGUGAUG | 818 |
| | | | AGUUAUAAAAUCACAGAGGGUG | 819 |
| | | | UGAUCAAGUUAUAAAAUCACAG | 820 |
| | | | CUGAGUAGGAGCUAAAAUAUUU | 821 |
| | | | UUGAUCAAGTTATAAAATCACA | 822 |
| | | | GGGCTTGGACAGAACTTACCGA | 823 |
| | | | CTCTGGCAGATTTCAACCGGGC | 824 |
| | | | ACCTGCTCTGGCAGATTTCAAC | 825 |
| | | | TACCTGCTCTGGCAGATTTCAA | 826 |
| | | | CTTGATGTTGGAGGTACCTGCT | 827 |
| | | | AATGCCATCTTCCTTGATGTTG | 828 |
| | | | AGAAATGCCATCTTCCTTGATG | 829 |
| | | | GGTGACACAACCTGTGGTTACT | 830 |
| | | | TGTTACTCTGGTGACACAACCT | 831 |
| | | | AGCTCCTACTCAGACTGTTACT | 832 |
| | | | CGGTAAGITCTGICCTCAAGGAAGATGGCA | 833 |
| | | | CTCATACCTTCTGCTTCAAGGAAGATGGCA | 834 |
| | | | CTCCAACATCAAGGAAGATGGCATTTCTAG | 835 |
| | | | AACATCAAGGAAGATGGCATT | 836 |
| | | | TCCAACATCAAGGAAGATGGC | 837 |
| | | | ACCTCCAACATCAAGGAAGAT | 838 |
| | | | GAGUAACAGUCUGAGUAGGAG | 839 |
| | | | UGUGUCACCAGAGUAACAGUC | 840 |
| | | | AACCACAGGUUGUGUCACCAG | 841 |
| | | | UUUCCUUAGUAACCACAGGUU | 842 |
| | | | GAGAUGGCAGUUUCCUUAGUA | 843 |
| | | | UUCUAGUUUGGAGAUGGCAGU | 844 |
| | | | AAGAUGGCAUUUCUAGUUUGG | 845 |
| | | | AACAUCAAGGAAGAUGGCAUU | 846 |
| | | | AGGUACCUCCAACAUCAAGGA | 847 |
| | | | CUGCCAGAGCAGGUACCUCCA | 848 |
| | | | CGGUUGAAAUCUGCCAGAGCA | 849 |
| | | | UGUCCAAGCCCGGUUGAAAUC | 850 |
| | | | CGGUAAGUUCUGUCCAAGCCC | 851 |
| | | | GAAAGCCAGUCGGUAAGUUCU | 852 |
| | | | AUCAAGCAGAGAAAGCCAGUC | 853 |
| | | | UUAUAACUUGAUCAAGCAGAG | 854 |
| | | | CUCUGUGAUUUUAUAACUUGA | 855 |
| | | | CACCAUCACCCUCUGUGAUUU | 856 |
| | | | CAAGGUCACCCACCAUCACCC | 857 |
| | | | UUGAUAUCCUCAAGGUCACCC | 858 |
| | | | GAUCAUCUCGUUGAUAUCCUC | 859 |
| | | | UCUGCUUGAUGAUCAUCUCGU | 860 |
| | | | GGCAUUUCUAGUUUGGAGAUG | 861 |
| | | | CAAGGAAGAUGGCAUUUCUAG | 862 |
| | | | CCUCCAACAUCAAGGAAGAUG | 863 |
| | | 52 | AGCCUCUUGAUUGCUGGUCUUGUUU | 864 |
| | | | GCCUCUUGAUUGCUGGUCUUGUUUU | 865 |
| | | | CCUCUUGAUUGCUGGUCUUGUUUUU | \|866 |
| | | | CCUCUUGAUUGCUGGUCUUG | 867 |
| | | | CUCUUGAUUGCUGGUCUUGUUUUUC | 868 |
| | | | UCUUGAUUGCUGGUCUUGUUUUUCA | 869 |
| | | | CUUGAUUGCUGGUCUUGUUUUUCAA | 870 |
| | | | UUGAUUGCUGGUCUUGUUUUUCAAA | 871 |
| | | | UGAUUGCUGGUCUUGUUUUUCAAAU | 872 |
| | | | GAUUGCUGGUCUUGUUUUUCAAAUU | 873 |
| | | | GAUUGCUGGUCUUGUUUUUC | 874 |
| | | | AUUGCUGGUCUUGUUUUUCAAAUUU | 875 |
| | | | UUGCUGGUCUUGUUUUUCAAAUUUU | 876 |
| | | | UGCUGGUCUUGUUUUUCAAAUUUUG | 877 |
| | | | GCUGGUCUUGUUUUUCAAAUUUUGG | 878 |
| | | | CUGGUCUUGUUUUUCAAAUUUUGGG | 879 |
| | | | UGGUCUUGUUUUUCAAAUUUUGGGC | 880 |
| | | | GGUCUUGUUUUUCAAAUUUUGGGCA | 881 |
| | | | GUCUUGUUUUUCAAAUUUUGGGCAG | 882 |
| | | | UCUUGUUUUUCAAAUUUUGGGCAGC | 883 |
| | | | CUUGUUUUUCAAAUUUUGGGCAGCG | 884 |
| | | | UUGUUUUUCAAAUUUUGGGCAGCGG | 885 |
| | | | UGUUUUUCAAAUUUUGGGCAGCGGU | 886 |
| | | | GUUUUUCAAAUUUUGGGCAGCGGUA | 887 |
| | | | UUUUUCAAAUUUUGGGCAGCGGUAA | 888 |
| | | | UUUUCAAAUUUUGGGCAGCGGUAAU | 889 |
| | | | UUUCAAAUUUUGGGCAGCGGUAAUG | 890 |
| | | | UUCAAAUUUUGGGCAGCGGUAAUGA | 891 |
| | | | UCAAAUUUUGGGCAGCGGUAAUGAG | 892 |
| | | | CAAAUUUUGGGCAGCGGUAAUGAGU | 893 |
| | | | AAAUUUUGGGCAGCGGUAAUGAGUU | 894 |
| | | | AAUUUUGGGCAGCGGUAAUGAGUUC | 895 |
| | | | AUUUUGGGCAGCGGUAAUGAGUUCU | 896 |
| | | | UUUUGGGCAGCGGUAAUGAGUUCUU | 897 |
| | | | UUUGGGCAGCGGUAAUGAGUUCUUC | 898 |
| | | | UUGGGCAGCGGUAAUGAGUUCUUCC | 899 |
| | | | UGGGCAGCGGUAAUGAGUUCUUCCA | 900 |
| | | | GGGCAGCGGUAAUGAGUUCUUCCAA | 901 |
| | | | GGCAGCGGUAAUGAGUUCUUCCAAC | 902 |
| | | | GCAGCGGUAAUGAGUUCUUCCAACU | 903 |
| | | | CAGCGGUAAUGAGUUCUUCCAACUG | 904 |
| | | | AGCGGUAAUGAGUUCUUCCAACUGG | 905 |
| | | | GCGGUAAUGAGUUCUUCCAACUGGG | 906 |
| | | | CGGUAAUGAGUUCUUCCAACUGGGG | 907 |
| | | | GGUAAUGAGUUCUUCCAACUGGGGA | 908 |
| | | | GGUAAUGAGUUCUUCCAACUGG | 909 |
| | | | GUAAUGAGUUCUUCCAACUGGGGAC | 910 |
| | | | UAAUGAGUUCUUCCAACUGGGGACG | 911 |
| | | | AAUGAGUUCUUCCAACUGGGGACGC | 912 |
| | | | AUGAGUUCUUCCAACUGGGGACGCC | 913 |
| | | | UGAGUUCUUCCAACUGGGGACGCCU | 914 |
| | | | GAGUUCUUCCAACUGGGGACGCCUC | 915 |
| | | | AGUUCUUCCAACUGGGGACGCCUCU | 916 |
| | | | GUUCUUCCAACUGGGGACGCCUCUG | 917 |
| | | | UUCUUCCAACUGGGGACGCCUCUGU | 918 |
| | | | UCUUCCAACUGGGGACGCCUCUGUU | 919 |
| | | | CUUCCAACUGGGGACGCCUCUGUUC | 920 |
| | | | UUCCAACUGGGGACGCCUCUGUUCC | 921 |
| | | | UCCAACUGGGGACGCCUCUGUUCCA | 922 |
| | | | CCAACUGGGGACGCCUCUGUUCCAA | 923 |
| | | | CAACUGGGGACGCCUCUGUUCCAAA | 924 |
| | | | AACUGGGGACGCCUCUGUUCCAAAU | 925 |
| | | | ACUGGGGACGCCUCUGUUCCAAAUC | 926 |
| | | | CUGGGGACGCCUCUGUUCCAAAUCC | 927 |
| | | | UGGGGACGCCUCUGUUCCAAAUCCU | 928 |
| | | | GGGGACGCCUCUGUUCCAAAUCCUG | 929 |
| | | | GGGACGCCUCUGUUCCAAAUCCUGC | 930 |
| | | | GGACGCCUCUGUUCCAAAUCCUGCA | 931 |
| | | | GACGCCUCUGUUCCAAAUCCUGCAU | 932 |
| | | 53 | CUCUGGCCUGUCCUAAGACCUGCUC | 933 |
| | | | UCUGGCCUGUCCUAAGACCUGCUCA | 934 |
| | | | CUGGCCUGUCCUAAGACCUGCUCAG | 935 |
| | | | UGGCCUGUCCUAAGACCUGCUCAGC | 936 |
| | | | GGCCUGUCCUAAGACCUGCUCAGCU | 937 |
| | | | GCCUGUCCUAAGACCUGCUCAGCUU | 938 |
| | | | CCUGUCCUAAGACCUGCUCAGCUUC | 939 |
| | | | CUGUCCUAAGACCUGCUCAGCUUCU | 940 |
| | | | UGUCCUAAGACCUGCUCAGCUUCUU | 941 |
| | | | GUCCUAAGACCUGCUCAGCUUCUUC | 942 |
| | | | UCCUAAGACCUGCUCAGCUUCUUCC | 943 |
| | | | CCUAAGACCUGCUCAGCUUCUUCCU | 944 |
| | | | CUAAGACCUGCUCAGCUUCUUCCUU | 945 |
| | | | UAAGACCUGCUCAGCUUCUUCCUUA | 946 |
| | | | AAGACCUGCUCAGCUUCUUCCUUAG | 947 |
| | | | AGACCUGCUCAGCUUCUUCCUUAGC | 948 |
| | | | GACCUGCUCAGCUUCUUCCUUAGCU | 949 |
| | | | ACCUGCUCAGCUUCUUCCUUAGCUU | 950 |
| | | | CCUGCUCAGCUUCUUCCUUAGCUUC | 951 |
| | | | CUGCUCAGCUUCUUCCUUAGCUUCC | 952 |
| | | | UGCUCAGCUUCUUCCUUAGCUUCCA | 953 |
| | | | GCUCAGCUUCUUCCUUAGCUUCCAG | 954 |
| | | | CUCAGCUUCUUCCUUAGCUUCCAGC | 955 |
| | | | UCAGCUUCUUCCUUAGCUUCCAGCC | 956 |
| | | | CAGCUUCUUCCUUAGCUUCCAGCCA | 957 |
| | | | AGCUUCUUCCUUAGCUUCCAGCCAU | 958 |
| | | | GCUUCUUCCUUAGCUUCCAGCCAUU | 959 |
| | | | CUUCUUCCUUAGCUUCCAGCCAUUG | 960 |
| | | | UUCUUCCUUAGCUUCCAGCCAUUGU | 961 |
| | | | UCUUCCUUAGCUUCCAGCCAUUGUG | 962 |
| | | | CUUCCUUAGCUUCCAGCCAUUGUGU | 963 |
| | | | UUCCUUAGCUUCCAGCCAUUGUGUU | 964 |
| | | | UCCUUAGCUUCCAGCCAUUGUGUUG | 965 |
| | | | CCUUAGCUUCCAGCCAUUGUGUUGA | 966 |
| | | | CUUAGCUUCCAGCCAUUGUGUUGAA | 967 |
| | | | UUAGCUUCCAGCCAUUGUGUUGAAU | 968 |
| | | | UAGCUUCCAGCCAUUGUGUUGAAUC | 969 |
| | | | AGCUUCCAGCCAUUGUGUUGAAUCC | 970 |
| | | | GCUUCCAGCCAUUGUGUUGAAUCCU | 971 |
| | | | CUUCCAGCCAUUGUGUUGAAUCCUU | 972 |
| | | | UUCCAGCCAUUGUGUUGAAUCCUUU | 973 |
| | | | UCCAGCCAUUGUGUUGAAUCCUUUA | 974 |
| | | | CCAGCCAUUGUGUUGAAUCCUUUAA | 975 |
| | | | CAGCCAUUGUGUUGAAUCCUUUAAC | 976 |
| | | | AGCCAUUGUGUUGAAUCCUUUAACA | 977 |
| | | | GCCAUUGUGUUGAAUCCUUUAACAU | 978 |
| | | | CCAUUGUGUUGAAUCCUUUAACAUU | 979 |
| | | | CAUUGUGUUGAAUCCUUUAACAUUU | 980 |
| | | | CCUUGGUUUCUGUGAUUUUCUU | 981 |
| | | | UCCUUAGCUUCCAGCCAUUGUG | 982 |
| | | | CUUGUACUUCAUCCCACUGAUU | 983 |
| | | | ACUGAUUCUGAAUUCUUUCAAC | 984 |
| | | | AGCCAAGCUUGAGUCAUGGAAG | 985 |
| | | | UUAGGACAGGCCAGAGCCAAGC | 986 |
| | | | GCAACAGUUGAAUGAAAUGUUA | 987 |
| | | | CCUUCAGAACCGGAGGCAACAG | 988 |
| | | | AGUUGAAAGAAUUCAGAAUCAG | 989 |
| | | | UUUUAUUCUAGUUGAAAGAAUU | 990 |
| | | | UUUUUCCUUUUAUUCUAGUUGA | 991 |
| | | | AAAGGUAUCUUUGAUACUAACC | 992 |
| | | | CCUUGGUUUCUGUGAUUUUCUU | 993 |
| | | | CUUUUGGAUUGCAUCUACUGUA | 994 |
| | | | UUUUGGAUUGCAUCUACUGUAU | 995 |
| | | | ACCCUCCUUCCAUGACUCAAGC | 996 |
| | | | CUUCCAUGACUCAAGCUUGGCU | 997 |
| | | | ACAUUUCAUUCAACUGUUGCCU | 998 |
| | | | UCAACUGUUGCCUCCGGUUCUG | 999 |
| | | | CCAAAAGAAAAUCACAGAAACC | 1000 |
| | | | GCCAAGCUUGAGUCAUGGAAGG | 1001 |
| | | | AGCCAAGCUUGAGUCAUGGAAG | 1002 |
| | | | CAGAGCCAAGCUUGAGUCAUGG | 1003 |
| | | | AGGCCAGAGCCAAGCUUGAGUC | 1004 |
| | | | AGAAGCUGAGCAGGUCUUAGGA | 1005 |
| | | | AAGGAAGAAGCUGAGCAGGUCU | 1006 |
| | | | GGAAGCUAAGGAAGAAGCUGAG | 1007 |
| | | | UUCAACACAAUGGCUGGAAGCU | 1008 |
| | | | GUUAAAGGAUUCAACACAAUGG | 1009 |
| | | | AAAUGUUAAAGGAUUCAACACA | 1010 |
| | | | GCAACAGUUGAAUGAAAUGUUA | 1011 |
| | | | UACAAGAACACCUUCAGAACCG | 1012 |
| | | | AAGUACAAGAACACCUUCAGAA | 1013 |
| | | | AGUUGAAAGAAUUCAGAAUCAG | 1014 |
| | | | UAGUUGAAAGAAUUCAGAAUCA | 1015 |
| | | 55 | CUGUUGCAGUAAUCUAUGAG | 1016 |
| | | | UGCAGUAAUCUAUGAGUUUC | 1017 |
| | | | GAGUCUUCUAGGAGCCUU | 1018 |
| | | | UGCCAUUGUUUCAUCAGCUCUUU | 1019 |
| | | | UCCUGUAGGACAUUGGCAGU | 1020 |
| | | | CUUGGAGUCUUCUAGGAGCC | 1021 |
| | | 57 | UAGGUGCCUGCCGGCUU | 1022 |
| | | | UUCAGCUGUAGCCACACC | 1023 |
| | | | CUGAACUGCUGGAAAGUCGCC | 1024 |
| | | | CUGGCUUCCAAAUGGGACCUGAAAAAGAAC | 1025 |
| | | 59 | CAAUUUUUCCCACUCAGUAUU | 1026 |
| | | | UUGAAGUUCCUGGAGUCUU | 1027 |
| | | | UCCUCAGGAGGCAGCUCUAAAU | 1028 |
| | | 62 | UGGCUCUCUCCCAGGG | 1029 |
| | | | GAGAUGGCUCUCUCCCAGGGACCCUGG | 1030 |
| | | | GGGCACUUUGUUUGGCG | 1031 |

In some embodiments, the ASO comprises a sequence selected from Tables 6-8. In some embodiments, the ASO comprises a sequence selected from SEQ ID NOs: 201-1031. In some embodiments, any of the nucleic acids of Table 6-8 can be linked to any 3E10 antibody or antigen-binding fragment thereof described herein via any linker described herein. In some embodiments, any of the nucleic acids of Table 6-8 is linked to any 3E10 antibody or antigen-binding fragment thereof described herein via a cathepsin L cleavable linker. In some embodiments, any of the nucleic acids of Table 6-8 is linked to any 3E10 antibody or antigen-binding fragment thereof described herein via a cleavable linker comprising a -Phe-Gln-dipeptide.

In some embodiments, any of the nucleic acids of Table 6-8 is linked to any 3E10 antibody or antigen-binding fragment thereof described herein via a cleavable linker comprising a -Val-Gln-dipeptide. In some embodiments, any of the nucleic acids of Table 6-8 is linked to any 3E10 antibody or antigen-binding fragment thereof described herein via a cleavable linker comprising a -Leu-Gln-dipeptide.

### D. Exemplary Linker-Antibodies

In embodiments, the present disclosure provides an antibody-oligonucleotide conjugate (AOC) having the formula A-(L-Pᵣ)_{q}, wherein: A is a 3E10 antibody or antigen-binding fragment thereof, L is a linker, P is an oligonucleotide moiety described herein, r is an integer from 1 to 4, and q is an integer from 1 to 16.

In some embodiments, L is a cathepsin-L cleavable linker, and A is an antibody or antigen-binding fragment thereof that comprises VL CDRs of SEQ ID NOs: 61, 62, and 63 and VH CDRs of SEQ ID NOs: 58, 59, and 60. In some embodiments, the cathepsin-L cleavable linker comprises a -Phe-Gln-dipeptide. In some embodiments, the cathepsin-L cleavable linker comprises a -Val-Gln-dipeptide. In some embodiments, the cathepsin-L cleavable linker comprises a -Leu-Gln-dipeptide. In some embodiments, the cathepsin-L cleavable linker comprises a -Tyr-Met-dipeptide. In some embodiments, the cathepsin-L cleavable linker comprises a -Phe-Arg-dipeptide. In some embodiments, the cathepsin-L cleavable linker comprises a -Phe-Gly-dipeptide. In some embodiments, the cathepsin-L comprises cleavable linker comprises a -Trp-Thr-dipeptide. In some embodiments, the cathepsin-L cleavable linker comprises a -Tyr-Gly-dipeptide. In some embodiments, the cathepsin-L cleavable linker comprises a -Phe-Thr-dipeptide. In some embodiments, the cathepsin-L cleavable linker comprises a -Val-Gly-dipeptide. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an AOC with a cathepsin-L cleavable linker and an antibody or antigen-binding fragment thereof that comprises VL CDRs of SEQ ID NOs: 61, 62, and 63 and VH CDRs of SEQ ID NOs: 58, 59, and 60, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the cancer cells express the ENT2 transporter protein. In some embodiments, the cancer cells express functional ENT2, wherein the ENT2 nucleoside salvage pathway is not disrupted such that nucleic acid molecules and the 3E10 antibody or antigen-binding fragment thereof with nucleic acid molecules can enter the cells via the ENT2 transporter. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cathepsin-L cleavable linker, and A is an antibody or antigen-binding fragment thereof that comprises VL CDRs of SEQ ID NOs: 9, 10, and 11 and VH CDRs of SEQ ID NOs: 15, 4, and 5. In some embodiments, the cathepsin-L cleavable linker comprises a -Phe-Gln-dipeptide. In some embodiments, the cathepsin-L cleavable linker comprises a -Val-Gln-dipeptide. In some embodiments, the cathepsin-L cleavable linker comprises a -Leu-Gln-dipeptide. In some embodiments, the cathepsin-L cleavable linker comprises a -Tyr-Met-dipeptide. In some embodiments, the cathepsin-L cleavable linker comprises a -Phe-Arg-dipeptide. In some embodiments, the cathepsin-L cleavable linker comprises a -Phe-Gly-dipeptide. In some embodiments, the cathepsin-L comprises cleavable linker comprises a -Trp-Thr-dipeptide. In some embodiments, the cathepsin-L cleavable linker comprises a -Tyr-Gly-dipeptide. In some embodiments, the cathepsin-L cleavable linker comprises a -Phe-Thr-dipeptide. In some embodiments, the cathepsin-L cleavable linker comprises a -Val-Gly-dipeptide. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an AOC with a cathepsin-L cleavable linker and an antibody or antigen-binding fragment thereof that comprises VL CDRs of SEQ ID NOs: 9, 10, and 11 and VH CDRs of SEQ ID NOs: 15, 4, and 5, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cathepsin-L cleavable linker, and A is an antibody or antigen-binding fragment thereof that comprises VL CDRs of SEQ ID NOs: 29, 10, and 11 and VH CDRs of SEQ ID NOs: 15, 26, and 5. In some embodiments, the cathepsin-L cleavable linker comprises a -Phe-Gln-dipeptide. In some embodiments, the cathepsin-L cleavable linker comprises a -Val-Gln-dipeptide. In some embodiments, the cathepsin-L cleavable linker comprises a -Leu-Gln-dipeptide. In some embodiments, the cathepsin-L cleavable linker comprises a -Tyr-Met-dipeptide. In some embodiments, the cathepsin-L cleavable linker comprises a -Phe-Arg-dipeptide. In some embodiments, the cathepsin-L cleavable linker comprises a -Phe-Gly-dipeptide. In some embodiments, the cathepsin-L comprises cleavable linker comprises a -Trp-Thr-dipeptide. In some embodiments, the cathepsin-L cleavable linker comprises a -Tyr-Gly-dipeptide. In some embodiments, the cathepsin-L cleavable linker comprises a -Phe-Thr-dipeptide. In some embodiments, the cathepsin-L cleavable linker comprises a -Val-Gly-dipeptide. In some embodiments, the antibody or antigen-binding fragment thereof further comprises a heavy chain variable region (VH) with an amino acid sequence having at least 95% sequence identity to SEQ ID NO:127 and a light chain variable region (VL) with an amino acid sequence having at least 95% sequence identity to SEQ ID NO:126. In some embodiments, the antibody or antigen-binding fragment thereof further comprises a VH with an amino acid sequence having at least 98% sequence identity to SEQ ID NO:127 and a VL with an amino acid sequence having at least 98% sequence identity to SEQ ID NO:126. In some embodiments, the antibody or antigen-binding fragment thereof further comprises a VH with an amino acid sequence having at least 99% sequence identity to SEQ ID NO:127 and a VL with an amino acid sequence having at least 99% sequence identity to SEQ ID NO:126. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an AOC with a cathepsin-L cleavable linker and an antibody or antigen-binding fragment thereof that comprises VL CDRs of SEQ ID NOs: 29, 10, and 11 and VH CDRs of SEQ ID NOs: 15, 26, and 5, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cathepsin-L cleavable linker, and A is an antibody or antigen-binding fragment thereof that comprises heavy chain variable region (VH) with an amino acid sequence of SEQ ID NO:69 and a light chain variable region (VL) with an amino acid sequence of SEQ ID NO:90. In some embodiments, the cathepsin-L cleavable linker comprises a -Phe-Gln-dipeptide. In some embodiments, the cathepsin-L cleavable linker comprises a -Val-Gln-dipeptide. In some embodiments, the cathepsin-L cleavable linker comprises a -Leu-Gln-dipeptide. In some embodiments, the cathepsin-L cleavable linker comprises a -Tyr-Met-dipeptide. In some embodiments, the cathepsin-L cleavable linker comprises a -Phe-Arg-dipeptide. In some embodiments, the cathepsin-L cleavable linker comprises a -Phe-Gly-dipeptide. In some embodiments, the cathepsin-L comprises cleavable linker comprises a -Trp-Thr-dipeptide. In some embodiments, the cathepsin-L cleavable linker comprises a -Tyr-Gly-dipeptide. In some embodiments, the cathepsin-L cleavable linker comprises a -Phe-Thr-dipeptide. In some embodiments, the cathepsin-L cleavable linker comprises a -Val-Gly-dipeptide. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an AOC with a cathepsin-L cleavable linker and an antibody or antigen-binding fragment thereof that comprises heavy chain variable region (VH) with an amino acid sequence of SEQ ID NO:69 and a light chain variable region (VL) with an amino acid sequence of SEQ ID NO:90, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker and an antibody or antigen-binding fragment thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker and an antibody or antigen-binding fragment thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H1-dependent antisense oligonucleotide. In some embodiments, the ASO mediates exon skipping.

In some embodiments, L is a cathepsin-L cleavable linker, and A is an antibody or antigen-binding fragment thereof that comprises heavy chain variable region (VH) with an amino acid sequence of SEQ ID NO:105 and a light chain variable region (VL) with an amino acid sequence of SEQ ID NO:117. In some embodiments, the cathepsin-L cleavable linker comprises a-Phe-Gln-dipeptide. In some embodiments, the cathepsin-L cleavable linker comprises a -Val-Gln-dipeptide. In some embodiments, the cathepsin-L cleavable linker comprises a -Leu-Gln-dipeptide. In some embodiments, the cathepsin-L cleavable linker comprises a -Tyr-Met-dipeptide. In some embodiments, the cathepsin-L cleavable linker comprises a -Phe-Arg-dipeptide. In some embodiments, the cathepsin-L cleavable linker comprises a -Phe-Gly-dipeptide. In some embodiments, the cathepsin-L comprises cleavable linker comprises a -Trp-Thr-dipeptide. In some embodiments, the cathepsin-L cleavable linker comprises a -Tyr-Gly-dipeptide. In some embodiments, the cathepsin-L cleavable linker comprises a -Phe-Thr-dipeptide. In some embodiments, the cathepsin-L cleavable linker comprises a -Val-Gly-dipeptide. In some embodiments, the cathepsin-L cleavable linker is attached to the antibody or antigen-binding fragment thereof at a lysine residue. In some embodiments, the cathepsin-L cleavable linker is attached to the antibody or antigen-binding fragment thereof at a cysteine residue. In some embodiments, the cathepsin-L cleavable linker is attached to the antibody or antigen-binding fragment thereof at a glutamine residue.

In some embodiments of an AOC with a cathepsin-L cleavable linker and an antibody or antigen-binding fragment thereof that comprises heavy chain variable region (VH) with an amino acid sequence of SEQ ID NO:105 and a light chain variable region (VL) with an amino acid sequence of SEQ ID NO:117, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker and an antibody or antigen-binding fragment thereof that comprises heavy chain variable region (VH) with an amino acid sequence of SEQ ID NO: 105 and a light chain variable region (VL) with an amino acid sequence of SEQ ID NO: 117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker and an antibody or antigen-binding fragment thereof that comprises Heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Phe-Gln-dipeptide, and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Phe-Gln-dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln-dipeptide is attached to the antibody or antigen-binding fragment thereof at a glutamine residue.

In some embodiments of an AOC with a cleavable linker comprising a -Phe-Gln-dipeptide and an antibody or antigen-binding fragment thereof that comprises VL CDRs of SEQ ID NOs: 61, 62, and 63 and VH CDRs of SEQ ID NOs: 58, 59, and 60, the cleavable linker comprising a -Phe-Gln-dipeptide is an un-branched linker. In some embodiments, the cleavable linker comprising a -Phe-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Phe-Gln-dipeptide and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Phe-Gln-dipeptide and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Phe-Gln-dipeptide, and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Phe-Gln-dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln-dipeptide is attached to the antibody or antigen-binding fragment thereof at a glutamine residue.

In some embodiments of an AOC with a cleavable linker comprising a -Phe-Gln-dipeptide and an antibody or antigen-binding fragment thereof that comprises VL CDRs of SEQ ID NOs: 9, 10, and 11 and VH CDRs of SEQ ID NOs: 15, 4, and 5, the cleavable linker comprising a-Phe-Gln-dipeptide is an un-branched linker. In some embodiments, the cleavable linker comprising a -Phe-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Phe-Gln-dipeptide and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Phe-Gln-dipeptide and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Phe-Gln-dipeptide, and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO:117. In some embodiments, the antibody or antigen-binding fragment thereof further comprises a VH with an amino acid sequence having at least 98% sequence identity to SEQ ID NO:105 and a VL with an amino acid sequence having at least 98% sequence identity to SEQ ID NO:117. In some embodiments, the antibody or antigen-binding fragment thereof further comprises a VH with an amino acid sequence having at least 99% sequence identity to SEQ ID NO: 105 and a VL with an amino acid sequence having at least 99% sequence identity to SEQ ID NO: 117. In some embodiments, the cleavable linker comprising a -Phe-Gln-dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln-dipeptide is attached to the antibody or antigen-binding fragment thereof at a glutamine residue.

In some embodiments of an AOC with a cleavable linker comprising a -Phe-Gln-dipeptide and an antibody or antigen-binding fragment thereof that comprises VL CDRs of SEQ ID NOs: 29, 10, and 11 and VH CDRs of SEQ ID NOs: 15, 26, and 5, the cleavable linker comprising a -Phe-Gln-dipeptide is an un-branched linker. In some embodiments, the cleavable linker comprising a -Phe-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Phe-Gln-dipeptide and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Phe-Gln-dipeptide and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Phe-Gln-dipeptide, and A is an antibody or antigen-binding fragment thereof that comprises a heavy chain variable region (VH) with an amino acid sequence of SEQ ID NO:69 and a light chain variable region (VL) with an amino acid sequence of SEQ ID NO:90. In some embodiments, the cleavable linker comprising a -Phe-Gln-dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln-dipeptide is attached to the antibody or antigen-binding fragment thereof at a glutamine residue.

In some embodiments of an AOC with a cleavable linker comprising a -Phe-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90, the cleavable linker comprising a-Phe-Gln-dipeptide is an un-branched linker. In some embodiments, the cleavable linker comprising a -Phe-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Phe-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Phe-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer.

In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Phe-Gln-dipeptide, and A is an antibody or antigen-binding fragment thereof that comprises a heavy chain variable region (VH) with an amino acid sequence of SEQ ID NO:105 and a light chain variable region (VL) with an amino acid sequence of SEQ ID NO:117. In some embodiments, the cleavable linker comprising a -Phe-Gln-dipeptide is attached to the antibody or antigen-binding fragment thereof at a lysine residue. In some embodiments, the cleavable linker comprising a -Phe-Gln-dipeptide is attached to the antibody or antigen-binding fragment thereof at a cysteine residue. In some embodiments, the cleavable linker comprising a -Phe-Gln-dipeptide is attached to the antibody or antigen-binding fragment thereof at a glutamine residue.

In some embodiments of an AOC with a cleavable linker comprising a -Phe-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117, the cleavable linker comprising a -Phe-Gln-dipeptide is an un-branched linker. In some embodiments, the cleavable linker comprising a -Phe-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Phe-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Phe-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Val-Gln-dipeptide, and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO: 69 and a VL with an amino acid sequence having at least 98% sequence identity to SEQ ID NO: 90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Val-Gln-dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln-dipeptide is attached to the antibody or antigen-binding fragment thereof at a glutamine residue.

In some embodiments of an AOC with a cleavable linker comprising a -Val-Gln-dipeptide and an antibody or antigen-binding fragment thereof that comprises VL CDRs of SEQ ID NOs: 61, 62, and 63 and VH CDRs of SEQ ID NOs: 58, 59, and 60, the cleavable linker comprising a -Val-Gln-dipeptide is an un-branched linker. In some embodiments, the cleavable linker comprising a -Val-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Val-Gln-dipeptide and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Val-Gln-dipeptide and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Val-Gln-dipeptide, and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO: 69 and a VL with an amino acid sequence having at least 98% sequence identity to SEQ ID NO: 90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Val-Gln-dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln-dipeptide is attached to the antibody or antigen-binding fragment thereof at a glutamine residue.

In some embodiments of an AOC with a cleavable linker comprising a -Val-Gln-dipeptide and an antibody or antigen-binding fragment thereof that comprises VL CDRs of SEQ ID NOs: 9, 10, and 11 and VH CDRs of SEQ ID NOs: 15, 4, and 5, the cleavable linker comprising a-Val-Gln-dipeptide is an un-branched linker. In some embodiments, the cleavable linker comprising a -Val-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Val-Gln-dipeptide and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Val-Gln-dipeptide and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Val-Gln-dipeptide, and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO:117. In some embodiments, the antibody or antigen-binding fragment thereof further comprises a VH with an amino acid sequence having at least 98% sequence identity to SEQ ID NO:105 and a VL with an amino acid sequence having at least 98% sequence identity to SEQ ID NO:117. In some embodiments, the antibody or antigen-binding fragment thereof further comprises a VH with an amino acid sequence having at least 99% sequence identity to SEQ ID NO: 105 and a VL with an amino acid sequence having at least 99% sequence identity to SEQ ID NO: 117. In some embodiments, the cleavable linker comprising a -Val-Gln-dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln-dipeptide is attached to the antibody or antigen-binding fragment thereof at a glutamine residue.

In some embodiments of an AOC with a cleavable linker comprising a -Val-Gln-dipeptide and an antibody or antigen-binding fragment thereof that comprises VL CDRs of SEQ ID NOs: 29, 10, and 11 and VH CDRs of SEQ ID NOs: 15, 26, and 5, the cleavable linker comprising a -Val-Gln-dipeptide is an un-branched linker. In some embodiments, the cleavable linker comprising a -Val-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Val-Gln-dipeptide and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Val-Gln-dipeptide and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Val-Gln-dipeptide, and A is an antibody or antigen-binding fragment thereof that comprises a heavy chain variable region (VH) with an amino acid sequence of SEQ ID NO:69 and a light chain variable region (VL) with an amino acid sequence of SEQ ID NO:90. In some embodiments, the cleavable linker comprising a -Val-Gln-dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln-dipeptide is attached to the antibody or antigen-binding fragment thereof at a glutamine residue.

In some embodiments of an AOC with a cleavable linker comprising a -Val-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90, the cleavable linker comprising a-Val-Gln-dipeptide is an un-branched linker. In some embodiments, the cleavable linker comprising a -Val-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Val-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Val-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Val-Gln-dipeptide, and A is an antibody or antigen-binding fragment thereof that comprises a heavy chain variable region (VH) with an amino acid sequence of SEQ ID NO:105 and a light chain variable region (VL) with an amino acid sequence of SEQ ID NO:117. In some embodiments, the cleavable linker comprising a -Val-Gln-dipeptide is attached to the antibody or antigen-binding fragment thereof at a lysine residue. In some embodiments, the cleavable linker comprising a -Val-Gln-dipeptide is attached to the antibody or antigen-binding fragment thereof at a cysteine residue. In some embodiments, the cleavable linker comprising a -Val-Gln-dipeptide is attached to the antibody or antigen-binding fragment thereof at a glutamine residue.

In some embodiments of an AOC with a cleavable linker comprising a -Val-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117, the cleavable linker comprising a-Val-Gln-dipeptide is an un-branched linker. In some embodiments, the cleavable linker comprising a -Val-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Val-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Val-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Leu-Gln-dipeptide, and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO: 69 and a VL with an amino acid sequence having at least 98% sequence identity to SEQ ID NO: 90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Leu-Gln-dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln-dipeptide is attached to the antibody or antigen-binding fragment thereof at a glutamine residue.

In some embodiments of an AOC with a cleavable linker comprising a -Leu-Gln-dipeptide and an antibody or antigen-binding fragment thereof that comprises VL CDRs of SEQ ID NOs: 61, 62, and 63 and VH CDRs of SEQ ID NOs: 58, 59, and 60, the cleavable linker comprising a -Leu-Gln-dipeptide is an un-branched linker. In some embodiments, the cleavable linker comprising a -Leu-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Leu-Gln-dipeptide and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Leu-Gln-dipeptide and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Leu-Gln-dipeptide, and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO: 69 and a VL with an amino acid sequence having at least 98% sequence identity to SEQ ID NO: 90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the cleavable linker comprising a -Leu-Gln-dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln-dipeptide is attached to the antibody or antigen-binding fragment thereof at a glutamine residue.

In some embodiments of an AOC with a cleavable linker comprising a -Leu-Gln-dipeptide and an antibody or antigen-binding fragment thereof that comprises VL CDRs of SEQ ID NOs: 9, 10, and 11 and VH CDRs of SEQ ID NOs: 15, 4, and 5, the cleavable linker comprising a-Leu-Gln-dipeptide is an un-branched linker. In some embodiments, the cleavable linker comprising a -Leu-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Leu-Gln-dipeptide and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Leu-Gln-dipeptide and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Leu-Gln-dipeptide, and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO:117. In some embodiments, the antibody or antigen-binding fragment thereof further comprises a VH with an amino acid sequence having at least 98% sequence identity to SEQ ID NO:105 and a VL with an amino acid sequence having at least 98% sequence identity to SEQ ID NO:117. In some embodiments, the antibody or antigen-binding fragment thereof further comprises a VH with an amino acid sequence having at least 99% sequence identity to SEQ ID NO: 105 and a VL with an amino acid sequence having at least 99% sequence identity to SEQ ID NO: 117. In some embodiments, the cleavable linker comprising a -Leu-Gln-dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln-dipeptide is attached to the antibody or antigen-binding fragment thereof at a glutamine residue.

In some embodiments of an AOC with a cleavable linker comprising a -Leu-Gln-dipeptide and an antibody or antigen-binding fragment thereof that comprises VL CDRs of SEQ ID NOs: 29, 10, and 11 and VH CDRs of SEQ ID NOs: 15, 26, and 5, the cleavable linker comprising a -Leu-Gln-dipeptide is an un-branched linker. In some embodiments, the cleavable linker comprising a -Leu-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Leu-Gln-dipeptide and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Leu-Gln-dipeptide and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Leu-Gln-dipeptide, and A is an antibody or antigen-binding fragment thereof that comprises a heavy chain variable region (VH) with an amino acid sequence of SEQ ID NO:69 and a light chain variable region (VL) with an amino acid sequence of SEQ ID NO:90. In some embodiments, the cleavable linker comprising a -Leu-Gln-dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln-dipeptide is attached to the antibody or antigen-binding fragment thereof at a glutamine residue.

In some embodiments of an AOC with a cleavable linker comprising a -Leu-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90, the cleavable linker comprising a-Leu-Gln-dipeptide is an un-branched linker. In some embodiments, the cleavable linker comprising a -Leu-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Leu-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Leu-Gln-dipeptide and an antibody or antigen-binding fragment thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a cleavable linker comprising a -Leu-Gln-dipeptide, and A is an antibody or antigen-binding fragment thereof that comprises a heavy chain variable region (VH) with an amino acid sequence of SEQ ID NO:105 and a light chain variable region (VL) with an amino acid sequence of SEQ ID NO:117. In some embodiments, the cleavable linker comprising a -Leu-Gln-dipeptide is attached to the antibody or antigen-binding fragment thereof at a lysine residue. In some embodiments, the cleavable linker comprising a -Leu-Gln-dipeptide is attached to the antibody or antigen-binding fragment thereof at a cysteine residue. In some embodiments, the cleavable linker comprising a -Leu-Gln-dipeptide is attached to the antibody or antigen-binding fragment thereof at a glutamine residue.

In some embodiments of an AOC with a cleavable linker comprising a -Leu-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117, the cleavable linker comprising a-Leu-Gln-dipeptide is an un-branched linker. In some embodiments, the cleavable linker comprising a -Leu-Gln-dipeptide is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cleavable linker comprising a-Leu-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cleavable linker comprising a -Leu-Gln-dipeptide and an antibody or antigen-binding fragment thereof that comprises a heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a disulfide cleavable linker, and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an AOC with a disulfide cleavable linker and an antibody or antigen-binding fragment thereof that comprises VL CDRs of SEQ ID NOs: 61, 62, and 63 and VH CDRs of SEQ ID NOs: 58, 59, and 60, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a disulfide cleavable linker, and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an AOC with a disulfide cleavable linker and an antibody or antigen-binding fragment thereof that comprises VL CDRs of SEQ ID NOs: 9, 10, and 11 and VH CDRs of SEQ ID NOs: 15, 4, and 5, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a disulfide cleavable linker, and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO: 117. In some embodiments, the antibody or antigen-binding fragment thereof further comprises a VH with an amino acid sequence having at least 98% sequence identity to SEQ ID NO: 105 and a VL with an amino acid sequence having at least 98% sequence identity to SEQ ID NO: 117. In some embodiments, the antibody or antigen-binding fragment thereof further comprises a VH with an amino acid sequence having at least 99% sequence identity to SEQ ID NO: 105 and a VL with an amino acid sequence having at least 99% sequence identity to SEQ ID NO: 117. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an AOC with a disulfide cleavable linker and an antibody or antigen-binding fragment thereof that comprises VL CDRs of SEQ ID NOs: 29, 10, and 11 and VH CDRs of SEQ ID NOs: 15, 26, and 5, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a disulfide cleavable linker, and A is an antibody or antigen-binding fragment thereof that comprises a heavy chain variable region (VH) with an amino acid sequence of SEQ ID NO:69 and a light chain variable region (VL) with an amino acid sequence of SEQ ID NO:90. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an AOC with a disulfide cleavable linker and an antibody or antigen-binding fragment thereof that comprises a heavy chain variable region (VH) with an amino acid sequence of SEQ ID NO:69 and a light chain variable region (VL) with an amino acid sequence of SEQ ID NO:90, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker and an antibody or antigen-binding fragment thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker and an antibody or antigen-binding fragment thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a disulfide cleavable linker, and A is an antibody or antigen-binding fragment thereof that comprises a heavy chain variable region (VH) with an amino acid sequence of SEQ ID NO:105 and a light chain variable region (VL) with an amino acid sequence of SEQ ID NO:117. In some embodiments, the disulfide cleavable linker is attached to the antibody or antigen-binding fragment thereof at a lysine residue. In some embodiments, the disulfide cleavable linker is attached to the antibody or antigen-binding fragment thereof at a cysteine residue. In some embodiments, the disulfide cleavable linker is attached to the antibody or antigen-binding fragment thereof at a glutamine residue.

In some embodiments of an AOC with a disulfide cleavable linker and an antibody or antigen-binding fragment thereof that comprises a heavy chain variable region (VH) with an amino acid sequence of SEQ ID NO: 105 and a light chain variable region (VL) with an amino acid sequence of SEQ ID NO:117, the disulfide cleavable linker is an un-branched linker. In some embodiments, the disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker and an antibody or antigen-binding fragment thereof that comprises a heavy chain variable region (VH) with an amino acid sequence of SEQ ID NO:105 and a light chain variable region (VL) with an amino acid sequence of SEQ ID NO: 117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker and an antibody or antigen-binding fragment thereof that comprises a heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a hindered disulfide cleavable linker, and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO: 90. In some embodiments, the antibody or antigen-binding fragment thereof further comprises a VH with an amino acid sequence having at least 98% sequence identity to SEQ ID a VH with an amino acid sequence having at least 99% sequence identity to SEQ ID NO:69 and a VL with an amino acid sequence having at least 99% sequence identity to SEQ ID NO:90. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an AOC with a hindered disulfide cleavable linker and an antibody or antigen-binding fragment thereof that comprises VL CDRs of SEQ ID NOs: 61, 62, and 63 and VH CDRs of SEQ ID NOs: 58, 59, and 60, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a hindered disulfide cleavable linker, and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an AOC with a hindered disulfide cleavable linker and an antibody or antigen-binding fragment thereof that comprises VL CDRs of SEQ ID NOs: 9, 10, and 11 and VH CDRs of SEQ ID NOs: 15, 4, and 5, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a hindered disulfide cleavable linker, and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO: 117. In some embodiments, the antibody or antigen-binding fragment thereof further comprises a VH with an amino acid sequence having at least 98% sequence identity to SEQ ID NO: 105 and a VL with an amino acid sequence having at least 98% sequence identity to SEQ ID NO: 117. In some embodiments, the antibody or antigen-binding fragment thereof further comprises a VH with an amino acid sequence having at least 99% sequence identity to SEQ ID NO: 105 and a VL with an amino acid sequence having at least 99% sequence identity to SEQ ID NO: 117. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an AOC with a hindered disulfide cleavable linker and an antibody or antigen-binding fragment thereof that comprises VL CDRs of SEQ ID NOs: 29, 10, and 11 and VH CDRs of SEQ ID NOs: 15, 26, and 5, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a hindered disulfide cleavable linker, and A is an antibody or antigen-binding fragment thereof that comprises a heavy chain variable region (VH) with an amino acid sequence of SEQ ID NO:69 and a light chain variable region (VL) with an amino acid sequence of SEQ ID NO:90. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an AOC with a hindered disulfide cleavable linker and an antibody or antigen-binding fragment thereof that comprises a heavy chain variable region (VH) with an amino acid sequence of SEQ ID NO:69 and a light chain variable region (VL) with an amino acid sequence of SEQ ID NO:90, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker and an antibody or antigen-binding fragment thereof that comprises a heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker and an antibody or antigen-binding fragment thereof that comprises a heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, L is a hindered disulfide cleavable linker, and A is an antibody or antigen-binding fragment thereof that comprises a heavy chain variable region (VH) with an amino acid sequence of SEQ ID NO:105 and a light chain variable region (VL) with an amino acid sequence of SEQ ID NO:117. In some embodiments, the hindered disulfide cleavable linker is attached to the antibody or antigen-binding fragment thereof at a lysine residue. In some embodiments, the hindered disulfide cleavable linker is attached to the antibody or antigen-binding fragment thereof at a cysteine residue. In some embodiments, the hindered disulfide cleavable linker is attached to the antibody or antigen-binding fragment thereof at a glutamine residue.

In some embodiments of an AOC with a hindered disulfide cleavable linker and an antibody or antigen-binding fragment thereof that comprises a heavy chain variable region (VH) with an amino acid sequence of SEQ ID NO:105 and a light chain variable region (VL) with an amino acid sequence of SEQ ID NO: 117, the hindered disulfide cleavable linker is an un-branched linker. In some embodiments, the hindered disulfide cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker and an antibody or antigen-binding fragment thereof that comprises a heavy chain variable region (VH) with an amino acid sequence of SEQ ID NO:105 and a light chain variable region (VL) with an amino acid sequence of SEQ ID NO:117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker and an antibody or antigen-binding fragment thereof that comprises a heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

### E. Exemplary Conjugation Chemistry-Linkers

In embodiments, the present disclosure provides an antibody-oligonucleotide conjugate (AOC) having the formula A-(L-Pᵣ)_{q}, wherein: A is a 3E10 antibody or antigen-binding fragment thereof, L is a linker, P is an oligonucleotide moiety described herein, r is an integer from 1 to 4, and q is an integer from 1 to 16.

In embodiments, L is a cathepsin-L cleavable linker attached to the 3E10 antibody or antigen-binding fragment thereof at a lysine residue. In some embodiments, the cathepsin-L cleavable linker comprises a -Phe-Gln-dipeptide. In embodiments, the cathepsin-L cleavable linker comprises a -Val-Gln-dipeptide. In embodiments, the cathepsin-L cleavable linker comprises a -Leu-Gln-dipeptide. In embodiments, the cathepsin-L comprises cleavable linker comprises a -Tyr-Met-dipeptide. In embodiments, the cathepsin-L comprises cleavable linker comprises a -Phe-Arg-dipeptide. In embodiments, the cathepsin-L comprises cleavable linker comprises a -Phe-Gly-dipeptide. In embodiments, the cathepsin-L comprises cleavable linker comprises a -Trp-Thr-dipeptide. In embodiments, the cathepsin-L comprises cleavable linker comprises a -Tyr-Gly-dipeptide. In embodiments, the cathepsin-L comprises cleavable linker comprises a -Phe-Thr-dipeptide. In embodiments, the cathepsin-L comprises cleavable linker comprises a -Val-Gly-dipeptide. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO: 105 and a light chain variable region (VL) with an amino acid sequence of SEQ ID NO:117.

In some embodiments of an AOC with a cathepsin-L cleavable linker attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker attached to the 3E10 antibody or antigen-binding fragment thereof at a lysine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker attached to the 3E10 antibody or antigen-binding fragment thereof at a cysteine residue. In some embodiments, the cathepsin-L cleavable linker comprises a -Phe-Gln-dipeptide. In embodiments, the cathepsin-L cleavable linker comprises a -Val-Gln-dipeptide. In embodiments, the cathepsin-L cleavable linker comprises a -Leu-Gln-dipeptide. In embodiments, the cathepsin-L comprises cleavable linker comprises a -Tyr-Met-dipeptide. In embodiments, the cathepsin-L comprises cleavable linker comprises a -Phe-Arg-dipeptide. In embodiments, the cathepsin-L comprises cleavable linker comprises a -Phe-Gly-dipeptide. In embodiments, the cathepsin-L comprises cleavable linker comprises a -Trp-Thr-dipeptide. In embodiments, the cathepsin-L comprises cleavable linker comprises a -Tyr-Gly-dipeptide. In embodiments, the cathepsin-L comprises cleavable linker comprises a -Phe-Thr-dipeptide. In embodiments, the cathepsin-L comprises cleavable linker comprises a -Val-Gly-dipeptide. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO: 105 and a light chain variable region (VL) with an amino acid sequence of SEQ ID NO:117.

In some embodiments of an AOC with a cathepsin-L cleavable linker attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker attached to the 3E10 antibody or antigen-binding fragment thereof at a cysteine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In some embodiments, the present disclosure provides pharmaceutical compositions comprising an antibody-oligonucleotide conjugate (AOC) having the formula A-(L-Pᵣ)_{q}, where: A is a 3E10 antibody or antigen-binding fragment thereof as described herein (e.g., as described in the section titled 3E10 Antibodies and Antigen-Binding Fragments), L is a linker, P is a payload moiety as described herein, r is an integer from 1 to 4 and q is an integer from 1 to 16.

In embodiments, L is a cathepsin-L cleavable linker attached to the 3E10 antibody or antigen-binding fragment thereof at a glutamine residue. In some embodiments, the cathepsin-L cleavable linker comprises a -Phe-Gln-dipeptide. In embodiments, the cathepsin-L cleavable linker comprises a -Val-Gln-dipeptide. In embodiments, the cathepsin-L cleavable linker comprises a -Leu-Gln-dipeptide. In embodiments, the cathepsin-L comprises cleavable linker comprises a -Tyr-Met-dipeptide. In embodiments, the cathepsin-L comprises cleavable linker comprises a -Phe-Arg-dipeptide. In embodiments, the cathepsin-L comprises cleavable linker comprises a -Phe-Gly-dipeptide. In embodiments, the cathepsin-L comprises cleavable linker comprises a -Trp-Thr-dipeptide. In embodiments, the cathepsin-L comprises cleavable linker comprises a -Tyr-Gly-dipeptide. In embodiments, the cathepsin-L comprises cleavable linker comprises a -Phe-Thr-dipeptide. In embodiments, the cathepsin-L comprises cleavable linker comprises a -Val-Gly-dipeptide. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO: 105 and a light chain variable region (VL) with an amino acid sequence of SEQ ID NO:117.

In some embodiments of an AOC with a cathepsin-L cleavable linker attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker attached to the 3E10 antibody or antigen-binding fragment thereof at a glutamine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Phe-Gln-dipeptide attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO: 69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Phe-Gln-dipeptide attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Phe-Gln-dipeptide attached to the 3E10 antibody or antigen-binding fragment thereof at a lysine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Phe-Gln-dipeptide attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Phe-Gln-dipeptide attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO: 69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Phe-Gln-dipeptide attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Phe-Gln-dipeptide attached to the 3E10 antibody or antigen-binding fragment thereof at a cysteine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Phe-Gln-dipeptide attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Phe-Gln-dipeptide attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Phe-Gln-dipeptide attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Phe-Gln-dipeptide attached to the 3E10 antibody or antigen-binding fragment thereof at a glutamine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Phe-Gln-dipeptide attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Val-Gln-dipeptide attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO: 69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Val-Gln-dipeptide attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Val-Gln-dipeptide attached to the 3E10 antibody or antigen-binding fragment thereof at a lysine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Val-Gln-dipeptide attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Val-Gln-dipeptide attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO: 69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Val-Gln-dipeptide attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Val-Gln-dipeptide attached to the 3E10 antibody or antigen-binding fragment thereof at a cysteine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Val-Gln-dipeptide attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Val-Gln-dipeptide attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Val-Gln-dipeptide attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Val-Gln-dipeptide attached to the 3E10 antibody or antigen-binding fragment thereof at a glutamine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Val-Gln-dipeptide attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Leu-Gln-dipeptide attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO: 69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Leu-Gln-dipeptide attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Leu-Gln-dipeptide attached to the 3E10 antibody or antigen-binding fragment thereof at a lysine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Leu-Gln-dipeptide attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Leu-Gln-dipeptide attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO: 69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Leu-Gln-dipeptide attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Leu-Gln-dipeptide attached to the 3E10 antibody or antigen-binding fragment thereof at a cysteine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Leu-Gln-dipeptide attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a cathepsin-L cleavable linker comprising a -Leu-Gln-dipeptide attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a cathepsin-L cleavable linker comprising a -Leu-Gln-dipeptide attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a cathepsin-L cleavable linker comprising a -Leu-Gln-dipeptide attached to the 3E10 antibody or antigen-binding fragment thereof at a glutamine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a cathepsin-L cleavable linker comprising a -Leu-Gln-dipeptide attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a disulfide cleavable linker attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a disulfide cleavable linker attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker attached to the 3E10 antibody or antigen-binding fragment thereof at a lysine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a disulfide cleavable linker attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a disulfide cleavable linker attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker attached to the 3E10 antibody or antigen-binding fragment thereof at a cysteine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a disulfide cleavable linker attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a disulfide cleavable linker attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a disulfide cleavable linker attached to the 3E10 antibody or antigen-binding fragment thereof at a glutamine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a disulfide cleavable linker attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a hindered disulfide cleavable linker attached to the 3E10 antibody or antigen-binding fragment thereof at a lysine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a hindered disulfide cleavable linker attached to the 3E10 antibody or antigen-binding fragment thereof at a lysine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker attached to the 3E10 antibody or antigen-binding fragment thereof at a lysine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker attached to the 3E10 antibody or antigen-binding fragment thereof at a lysine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a hindered disulfide cleavable linker attached to the 3E10 antibody or antigen-binding fragment thereof at a cysteine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO:117.

In some embodiments of an AOC with a hindered disulfide cleavable linker attached to the 3E10 antibody or antigen-binding fragment thereof at a cysteine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker attached to the 3E10 antibody or antigen-binding fragment thereof at a cysteine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker attached to the 3E10 antibody or antigen-binding fragment thereof at a cysteine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

In embodiments, L is a hindered disulfide cleavable linker attached to the 3E10 antibody or antigen-binding fragment thereof at a glutamine residue. In some embodiments, the antibody or antigen-binding fragment thereof comprises VL CDRs of SEQ ID NOs: 61, 62, and 63 and VH CDRs of SEQ ID NOs: 58, 59, and 60. In some embodiments, the antibody or antigen-binding fragment thereof comprises VL CDRs of SEQ ID NOs: 9, 10, and 11 and VH CDRs of SEQ ID NOs: 15, 4, and 5. In some embodiments, the antibody or antigen-binding fragment thereof comprises VL CDRs of SEQ ID NOs: 29, 10, and 11 and VH CDRs of SEQ ID NOs: 15, 26, and 5. In some embodiments, the antibody or antigen-binding fragment thereof comprises a heavy chain variable region (VH) with an amino acid sequence of SEQ ID NO:69 and a light chain variable region (VL) with an amino acid sequence of SEQ ID NO:90. In some embodiments, the antibody or antigen-binding fragment thereof comprises a heavy chain variable region (VH) with an amino acid sequence of SEQ ID NO:105 and a light chain variable region (VL) with an amino acid sequence of SEQ ID NO: 117.

In some embodiments of an AOC with a hindered disulfide cleavable linker attached to the 3E10 antibody or antigen-binding fragment thereof at a glutamine residue, the cathepsin-L cleavable linker is an un-branched linker. In some embodiments, the cathepsin-L cleavable linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with a hindered disulfide cleavable linker attached to the 3E10 antibody or antigen-binding fragment thereof at a glutamine residue are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with a hindered disulfide cleavable linker attached to the 3E10 antibody or antigen-binding fragment thereof at a glutamine residue, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the oligonucleotide (P) is an siRNA. In some embodiments, the oligonucleotide (P) is an antisense oligonucleotide (ASO). In some embodiments, the ASO is an RNase H₁-dependent antisense oligonucleotide. In some embodiments, the ASO mediates exon skipping.

### F. Exemplary AOCs

In embodiments, the present disclosure provides an antibody-oligonucleotide conjugate (AOC) having the formula A-(L-Pᵣ)_{q}, where: A is a 3E10 antibody or antigen-binding fragment thereof (e.g., as described in the section titled 3E10 Antibodies and Antigen-Binding Fragments), L is a linker as described herein, P is an oligonucleotide moiety described herein, r is an integer from 1 to 4, and q is an integer from 1 to 16.

In some embodiments, P is an siRNA, and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the siRNA is attached to the antibody or antigen-binding fragment thereof at a lysine residue. In some embodiments, the siRNA is attached to the antibody or antigen-binding fragment thereof at a cysteine residue. In some embodiments, the siRNA is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with an siRNA and an antibody or antigen-binding fragment thereof that comprises VL CDRs of SEQ ID NOs: 61, 62, and 63 and VH CDRs of SEQ ID NOs: 58, 59, and 60, the linker connecting the antibody or antigen binding fragment thereof to the siRNA is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with an siRNA and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with an siRNA and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is an siRNA, and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the siRNA is attached to the antibody or antigen-binding fragment thereof at a lysine residue. In some embodiments, the siRNA is attached to the antibody or antigen-binding fragment thereof at a cysteine residue. In some embodiments, the siRNA is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with an siRNA and an antibody or antigen-binding fragment thereof that comprises VL CDRs of SEQ ID NOs: 9, 10, and 11 and VH CDRs of SEQ ID NOs: 15, 4, and 5, the linker connecting the antibody or antigen binding fragment thereof to the siRNA is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with an siRNA and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with an siRNA and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is an siRNA, and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO:117. In some embodiments, the antibody or antigen-binding fragment thereof further comprises a VH with an amino acid sequence having at least 98% sequence identity to SEQ ID NO:105 and a VL with an amino acid sequence having at least 98% sequence identity to SEQ ID NO:117. In some embodiments, the antibody or antigen-binding fragment thereof further comprises a VH with an amino acid sequence having at least 99% sequence identity to SEQ ID NO:105 and a VL with an amino acid sequence having at least 99% sequence identity to SEQ ID NO:117. In some embodiments, the siRNA is attached to the antibody or antigen-binding fragment thereof at a lysine residue. In some embodiments, the siRNA is attached to the antibody or antigen-binding fragment thereof at a cysteine residue. In some embodiments, the siRNA is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with an siRNA and an antibody or antigen-binding fragment thereof that comprises VL CDRs of SEQ ID NOs: 29, 10, and 11 and VH CDRs of SEQ ID NOs: 15, 26, and 5, the linker connecting the antibody or antigen binding fragment thereof to the siRNA is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with an siRNA and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with an siRNA and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is an siRNA, and A is an antibody or antigen-binding fragment thereof that comprises heavy chain variable region (VH) with an amino acid sequence of SEQ ID NO:69 and a light chain variable region (VL) with an amino acid sequence of SEQ ID NO: 90. In some embodiments, the siRNA is attached to the antibody or antigen-binding fragment thereof at a lysine residue. In some embodiments, the siRNA is attached to the antibody or antigen-binding fragment thereof at a cysteine residue. In some embodiments, the siRNA is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with an siRNA and an antibody or antigen-binding fragment thereof that comprises heavy chain variable region (VH) with an amino acid sequence of SEQ ID NO:69 and a light chain variable region (VL) with an amino acid sequence of SEQ ID NO:90, the linker connecting the antibody or antigen binding fragment thereof to the siRNA is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with an siRNA and an antibody or antigen-binding fragment thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with an siRNA and an antibody or antigen-binding fragment thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is an siRNA, and A is an antibody or antigen-binding fragment thereof that comprises heavy chain variable region (VH) with an amino acid sequence of SEQ ID NO:105 and a light chain variable region (VL) with an amino acid sequence of SEQ ID NO: 117. In some embodiments, the siRNA is attached to the antibody or antigen-binding fragment thereof at a lysine residue. In some embodiments, the siRNA is attached to the antibody or antigen-binding fragment thereof at a cysteine residue. In some embodiments, the siRNA is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with an siRNA and an antibody or antigen-binding fragment thereof that comprises heavy chain variable region (VH) with an amino acid sequence of SEQ ID NO:105 and a light chain variable region (VL) with an amino acid sequence of SEQ ID NO: 117, the linker connecting the antibody or antigen binding fragment thereof to the siRNA is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with an siRNA and an antibody or antigen-binding fragment thereof that comprises heavy chain variable region (VH) with an amino acid sequence of SEQ ID NO:105 and a light chain variable region (VL) with an amino acid sequence of SEQ ID NO:117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with an siRNA and an antibody or antigen-binding fragment thereof that comprises Heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

In some embodiments, P is an antisense oligonucleotide (ASO), and A is an antibody or antigen-binding fragment thereof that comprises VL CDRs of SEQ ID NOs: 61, 62, and 63 and VH CDRs of SEQ ID NOs: 58, 59, and 60. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO: 90. In some embodiments, the antibody or antigen-binding fragment thereof further comprises a VH with an amino acid sequence having at least 98% sequence identity to SEQ ID a VH with an amino acid sequence having at least 99% sequence identity to SEQ ID NO:69 and a VL with an amino acid sequence having at least 99% sequence identity to SEQ ID NO:90. In some embodiments, the ASO is attached to the antibody or antigen-binding fragment thereof at a lysine residue. In some embodiments, the ASO is attached to the antibody or antigen-binding fragment thereof at a cysteine residue. In some embodiments, the ASO is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with ASO and an antibody or antigen-binding fragment thereof that comprises VL CDRs of SEQ ID NOs: 61, 62, and 63 and VH CDRs of SEQ ID NOs: 58, 59, and 60, the linker connecting the antibody or antigen binding fragment thereof to the ASO is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with ASO and an antibody or antigen-binding fragment thereof that comprises VL CDRs of SEQ ID NOs: 61, 62, and 63 and VH CDRs of SEQ ID NOs: 58, 59, and 60 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with ASO and an antibody or antigen-binding fragment thereof that comprises VL CDRs of SEQ ID NOs: 61, 62, and 63 and VH CDRs of SEQ ID NOs: 58, 59, and 60, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the ASO is an RNase H₁-dependent antisense oligonucleotide. In some embodiments, the ASO mediates exon skipping.

In some embodiments, P is an antisense oligonucleotide (ASO), and A is an antibody or antigen-binding fragment thereof that comprises VL CDRs of SEQ ID NOs: 9, 10, and 11 and VH CDRs of SEQ ID NOs: 15, 4, and 5. In some embodiments, the antibody or antigen-binding fragment thereof further comprises a heavy chain variable region (VH) with an amino acid sequence having at least 95% sequence identity to SEQ ID NO:69 and a light chain variable region (VL) with an amino acid sequence having at least 95% sequence identity to SEQ ID NO:90. In some embodiments, the antibody or antigen-binding fragment thereof further comprises a VH with an amino acid sequence having at least 98% sequence identity to SEQ ID NO:69 and a VL with an amino acid sequence having at least 98% sequence identity to SEQ ID NO:90. In some embodiments, the antibody or antigen-binding fragment thereof further comprises a VH with an amino acid sequence having at least 99% sequence identity to SEQ ID NO:69 and a VL with an amino acid sequence having at least 99% sequence identity to SEQ ID NO:90. In some embodiments, the ASO is attached to the antibody or antigen-binding fragment thereof at a lysine residue. In some embodiments, the ASO is attached to the antibody or antigen-binding fragment thereof at a cysteine residue. In some embodiments, the ASO is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with ASO and an antibody or antigen-binding fragment thereof that comprises VL CDRs of SEQ ID NOs: 9, 10, and 11 and VH CDRs of SEQ ID NOs: 15, 4, and 5, the linker connecting the antibody or antigen binding fragment thereof to the ASO is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with ASO and an antibody or antigen-binding fragment thereof that comprises VL CDRs of SEQ ID NOs: 9, 10, and 11 and VH CDRs of SEQ ID NOs: 15, 4, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with ASO and an antibody or antigen-binding fragment thereof that comprises VL CDRs of SEQ ID NOs: 9, 10, and 11 and VH CDRs of SEQ ID NOs: 15, 4, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the ASO is an RNase H₁-dependent antisense oligonucleotide. In some embodiments, the ASO mediates exon skipping.

In some embodiments, P is an antisense oligonucleotide (ASO), and A is an antibody or antigen-binding fragment thereof that comprises VL CDRs of SEQ ID NOs: 29, 10, and 11 and VH CDRs of SEQ ID NOs: 15, 26, and 5. In some embodiments, the antibody or antigen-binding fragment thereof further comprises a heavy chain variable region (VH) with an amino acid sequence having at least 95% sequence identity to SEQ ID NO:105 and a light chain variable region (VL) with an amino acid sequence having at least 95% sequence identity to SEQ ID NO: 117. In some embodiments, the antibody or antigen-binding fragment thereof further comprises a VH with an amino acid sequence having at least 98% sequence identity to SEQ ID NO: 105 and a VL with an amino acid sequence having at least 98% sequence identity to SEQ ID NO: 117. In some embodiments, the antibody or antigen-binding fragment thereof further comprises a VH with an amino acid sequence having at least 99% sequence identity to SEQ ID NO: 105 and a VL with an amino acid sequence having at least 99% sequence identity to SEQ ID NO: 117. In some embodiments, the ASO is attached to the antibody or antigen-binding fragment thereof at a lysine residue. In some embodiments, the ASO is attached to the antibody or antigen-binding fragment thereof at a cysteine residue. In some embodiments, the ASO is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with ASO and an antibody or antigen-binding fragment thereof that comprises VL CDRs of SEQ ID NOs: 29, 10, and 11 and VH CDRs of SEQ ID NOs: 15, 26, and 5, the linker connecting the antibody or antigen binding fragment thereof to the ASO is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with ASO and an antibody or antigen-binding fragment thereof that comprises VL CDRs of SEQ ID NOs: 29, 10, and 11 and VH CDRs of SEQ ID NOs: 15, 26, and 5 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with ASO and an antibody or antigen-binding fragment thereof that comprises VL CDRs of SEQ ID NOs: 29, 10, and 11 and VH CDRs of SEQ ID NOs: 15, 26, and 5, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the ASO is an RNase H₁-dependent antisense oligonucleotide. In some embodiments, the ASO mediates exon skipping.

In some embodiments, P is an antisense oligonucleotide (ASO), and A is an antibody or antigen-binding fragment thereof that comprises heavy chain variable region (VH) with an amino acid sequence of SEQ ID NO:69 and a light chain variable region (VL) with an amino acid sequence of SEQ ID NO:90. In some embodiments, the ASO is attached to the antibody or antigen-binding fragment thereof at a lysine residue. In some embodiments, the ASO is attached to the antibody or antigen-binding fragment thereof at a cysteine residue. In some embodiments, the ASO is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with ASO and an antibody or antigen-binding fragment thereof that comprises heavy chain variable region (VH) with an amino acid sequence of SEQ ID NO:69 and a light chain variable region (VL) with an amino acid sequence of SEQ ID NO: 90, the linker connecting the antibody or antigen binding fragment thereof to the ASO is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with ASO and an antibody or antigen-binding fragment thereof that comprises heavy chain variable region (VH) with an amino acid sequence of SEQ ID NO:69 and a light chain variable region (VL) with an amino acid sequence of SEQ ID NO:90 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with ASO and an antibody or antigen-binding fragment thereof that comprises heavy chain variable region (VH) with an amino acid sequence of SEQ ID NO:69 and a light chain variable region (VL) with an amino acid sequence of SEQ ID NO:90, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the ASO is an RNase H₁-dependent antisense oligonucleotide. In some embodiments, the ASO mediates exon skipping.

In some embodiments, P is an antisense oligonucleotide (ASO), and A is an antibody or antigen-binding fragment thereof that comprises heavy chain variable region (VH) with an amino acid sequence of SEQ ID NO:105 and a light chain variable region (VL) with an amino acid sequence of SEQ ID NO:117. In some embodiments, the ASO is attached to the antibody or antigen-binding fragment thereof at a lysine residue. In some embodiments, the ASO is attached to the antibody or antigen-binding fragment thereof at a cysteine residue. In some embodiments, the ASO is attached to the antibody or antigen-binding fragment thereof at a glutamine residue. In some embodiments, the linker is a cathepsin-L cleavable linker. In some embodiments, the linker is a disulfide cleavable linker. In some embodiments, the linker is a hindered disulfide linker.

In some embodiments of an AOC with ASO and an antibody or antigen-binding fragment thereof that comprises heavy chain variable region (VH) with an amino acid sequence of SEQ ID NO:105 and a light chain variable region (VL) with an amino acid sequence of SEQ ID NO: 117, the linker connecting the antibody or antigen binding fragment thereof to the ASO is an un-branched linker. In some embodiments, the linker is a branched linker. In some embodiments, the branched linker has two arms. In some embodiments, the branched linker has three arms. In some embodiments, the branched linker has four arms. In some embodiments, the branched linker has at least 2 arms, at least 3 arms, at least 4 arms, or more. In some embodiments, the branched linker has from 2 to 6 arms, from 2 to 5 arms, from 2 to 4 arms, from 2 to 3 arms, from 3 to 6 arms, from 3 to 5 arms, from 3 to 4 arms, from 4 to 6 arms, or from 5 to 6 arms.

In some embodiments, compositions of an AOC with ASO and an antibody or antigen-binding fragment thereof that comprises heavy chain variable region (VH) with an amino acid sequence of SEQ ID NO:105 and a light chain variable region (VL) with an amino acid sequence of SEQ ID NO:117 are provided. In some such compositions, the average drug to antibody ratio (DAR) for AOC in the composition at least 2. In some such compositions, the average DAR for AOC in the composition is at least 4. In some such compositions, the average DAR for AOC in the composition is at least 6. In some such compositions, the average DAR for AOC in the composition is at least 8. In some such compositions, the average DAR for AOC in the composition is at least 2, at least 3, at least 4, at least 5, at least 6, at least 7, at least 8, at least 9, at least 10, or greater. In some such compositions, the average DAR for AOC in the composition is at least 6.

In some embodiments, methods are provided for treating cancer by administering, to a subject in need thereof, a therapeutically effective amount of an AOC with ASO and an antibody or antigen-binding fragment thereof that comprises Heavy chain variable region (VH) with an amino acid sequence of SEQ ID NO:105 and a light chain variable region (VL) with an amino acid sequence of SEQ ID NO:117, as described above. In some embodiments, the cancer is a colorectal cancer. In some embodiments, the cancer is an ovarian cancer. In some embodiments, the cancer is a breast cancer. In some embodiments, the cancer is a pancreatic cancer. In some embodiments, the cancer is a non-small cell lung cancer. In some embodiments, the cancer is a biliary tract cancer. In some embodiments, the cancer is a gastric cancer. In some embodiments, the cancer is a cervical cancer. In yet other some embodiments, the cancer is colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor. In some embodiments, the ASO is an RNase H₁-dependent antisense oligonucleotide. In some embodiments, the ASO mediates exon skipping.

### IV. Pharmaceutical Compositions

In some aspects, the antibody-oligonucleotide conjugate (AOC) described herein can be used therapeutically in combination with a pharmaceutically acceptable carrier.

In embodiments, the present disclosure provides a pharmaceutical composition comprising an antibody-oligonucleotide conjugate (AOC) having the formula A-(L-Pᵣ)_{q}, wherein: A is a 3E10 antibody or antigen-binding fragment thereof, L is a linker, P is an oligonucleotide as described herein, r is an integer from 1 to 4, and q is an integer from 1 to 16; wherein the 3E10 antibody or antigen-binding fragment thereof comprises (a) a light chain variable region (VL) complementarity determining region (CDR) 1 comprising the amino acid sequence of X1ASX2X3VSTSSYSYX4X5, where X1 is K, R, or H, X2 is K, R, or H, X3 is T or S, X4 is M or L, and X5 is K, R, H, or A (SEQ ID NO:61), (b) a VL CDR2 comprising the amino acid sequence of YASYLXIS, where X¹ is D, E, N, or Q (SEQ ID NO:62), and (c) a VL CDR3 comprising the amino acid sequence of QX1SX2X3FPWT, where X1 is K, R, or H, X2 is K, R, or H, and X3 is D or E (SEQ ID NO:63), and (d) a heavy chain variable region (VH) CDR1 comprising the amino acid sequence of X1YGMX2, where X1 is D, E, N, Q, R, or K and X2 is K, R, or H (SEQ ID NO:58), (e) a VH CDR2 comprising the amino acid sequence of YISSX1SSTIYYAX2X3VX4G, where X1 is G or S, X2 is D or E, X3 is T or S, and X4 is K, R, or H (SEQ ID NO:59), and (f) a VH CDR3 comprising the amino acid sequence of X1GLLLX2Y, where X1 is K, R, or H, and X2 is D or E (SEQ ID NO:60), and a pharmaceutically acceptable carrier.

In embodiments, the present disclosure provides a pharmaceutical composition comprising an antibody-oligonucleotide conjugate (AOC) having the formula A-(L-Pᵣ)_{q}, wherein: A is a 3E10 antibody or antigen-binding fragment thereof, L is a linker, P is an oligonucleotide as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of NYGMH (SEQ ID NO: 15), and a pharmaceutically acceptable carrier. In embodiments, the antibody or antigen-binding fragment thereof comprises:
(a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5), and a pharmaceutically acceptable carrier. In embodiments, the antibody or antigen-binding fragment thereof comprises a light chain variable region (VL) comprising an amino acid sequence that is identical to SEQ ID NO:21. In embodiments, the antibody or antigen-binding fragment thereof comprises a heavy chain variable region (VH) comprising an amino acid sequence that is identical to SEQ ID NO:14. In embodiments, the antibody or antigen-binding fragment thereof comprises a full length light chain (LC) comprising an amino acid sequence that is identical to SEQ ID NO:20. In embodiments, the antibody or antigen-binding fragment thereof comprises a full length heavy chain (HC) comprising an amino acid sequence that is identical to SEQ ID NO:13.

In embodiments of pharmaceutical compositions comprising AOCs provided herein, the amino acid residue corresponding with N31 of the heavy chain CDR1 is substituted with R. In embodiments, the amino acid residue corresponding with N31 of the heavy chain CDR1 is substituted with L. In embodiments, the amino acid residue corresponding with N31 of the heavy chain CDR1 is substituted with K.

In embodiments of pharmaceutical compositions comprising an AOC provided herein, the antibody or antigen-binding fragment thereof is a murine, a chimeric, a humanized, or a human antibody.

In embodiments of the pharmaceutical composition comprising an antibody-oligonucleotide conjugate (AOC) provided herein, L is a cleavable linker. In embodiments, L is selected from the succinyl, O-succinyl, 4-succinimidyl-oxycarbonyl-a-(2-pyridyldithio) toluene, sulfosuccinimidyl 6-(3′-(2-pyridyldithio) propionamido) hexanoate, N-succinimidyl-3-(−2-pyridyldithio)-proprionate, succinimidyl 6-(3 (2-pyridyldithio) propionamido) hexanoate, 3-(2-pyridyldithio)-propionyl hydrazide, S-(2-thiopyridyl)-L-cysteine, N-succinimidyl 4-(2-pyridyldithio) butanoate (SPDB), N-succinimidyl 4-(2-pyridyldithio)-2-sulfobutanoate (sulfo-SPDB), N-succinimidyl 4-(2-pyridyldithio) pentanoate (SPP), CL2A, maleimidocaproyl-valine-citrulline-p-aminobenzoyloxycarbonyl (MC-vc-PAB) and N-succinimidyl 4-(2-pyridyldithio)-2-sulfopentanoate (sulfo-SPP). In embodiments of the pharmaceutical composition comprising an antibody-oligonucleotide conjugate (AOC) provided herein, L is a non-cleavable linker. In embodiments, L is selected from N-succinimidyl (4-iodoacetyl)-aminobenzoate, sulfosuccinimidyl(4-iodoacetyl)-aminobenzoate, dichlorotriazinic acid, N-succinimidyl-[(N-maleimidopropionamido)-tetraethyleneglycol]ester (NHS-PEG4-maleimide), N-succinimidyl 4-(maleimidomethyl)cyclohexanecarboxylate (SMCC), or N-sulfosuccinimidyl 4-(maleimidomethyl)cyclohexanecarboxylate (sulfoSMCC).

In embodiments of the pharmaceutical composition comprising an antibody-oligonucleotide conjugate (AOC), P is an siRNA, an siRNA molecule that targets cMyc mRNA, an antisense oligonucleotide (ASO), or an ASO that targets a cMyc gene.

In embodiments, the present disclosure provides a pharmaceutical composition comprising an antibody-oligonucleotide conjugate (AOC) having the formula A-(L-Pᵣ)_{q}, wherein: A is a humanized 3E10 antibody or antigen-binding fragment thereof comprising: (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO: 10), and (c) a VL CDR3 comprising the amino acid sequence of QHSREFPWT (SEQ ID NO:11), and (d) a heavy chain variable region (VH) CDR1 comprising the amino acid sequence of NYGMH (SEQ ID NO:15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5), L is a linker described herein, and P is an siRNA molecule that targets cMyc mRNA, and a pharmaceutically acceptable carrier.

In embodiments, the present disclosure provides a pharmaceutical composition comprising an AOC disclosed herein that has the formula A-(L-Pᵣ)_{q}, wherein: A is a 3E10 antibody or antigen-binding fragment thereof, L is a linker, P is an oligonucleotide as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of XYGMH (SEQ ID NO: 16), wherein X=D, N, R, L, or K and corresponds to an amino acid residue at position 31 of the 3E10 heavy chain, and wherein D31 is wild-type 3E10 CDR1, and a pharmaceutically acceptable carrier.

In some aspects of pharmaceutical compositions comprising the antibody-oligonucleotide conjugate (AOC) provided herein, the amino acid residue corresponding with N31 of the heavy chain CDR1 is substituted with R. In some aspects, the amino acid residue corresponding with N31 of the heavy chain CDR 1 is substituted with L. In some aspects, the amino acid residue corresponding with N31 of the heavy chain CDR1 is substituted with K.

In some aspects of the pharmaceutical composition comprising an 3E10 antibody-oligonucleotide conjugate (AOC) provided herein, the 3E10 antibody or antigen-binding fragment thereof is murine, chimeric, humanized, or human.

In some aspects of the pharmaceutical composition comprising an antibody-oligonucleotide conjugate (AOC) provided herein, comprises a cleavable linker. In some aspects, the cleavable linker is an acid-labile linker, a protease-sensitive linker, a photo-labile linker, or a disulfide-containing linker. In some aspects, the cleavable linker is selected from the group consisting of SPDB (CAS: 115088-06-7) or SPP (CAS: 341498-08-6). In some aspects, the cleavable linker is a homo-bi-functional linker, optionally comprising an alkyl or polyethylene glycol (PEG) chain. In some aspects, the cleavable homo-bi-functional linker is selected from the group consisting of DSP (Lomant's Reagent) (CAS: 57757-57-0) and Acid-PEG4-S-S-PEG4-Acid (CAS: 2055015-40-0).

In some aspects of the pharmaceutical composition comprising an antibody-oligonucleotide conjugate (AOC) provided herein, comprises a non-cleavable linker. In some aspects, the non-cleavable linker is selected from DBCO-C6-NHS ester (CAS: 1384870-47-6) and DBCO-PEG8-NHS ester. In some aspects, the non-cleavable linker is a homo-bi-functional linker, optionally comprising an alkyl or polyethylene glycol (PEG) chain. In some aspects, the non-cleavable homo-bi-functional linker is selected from DSS (CAS: 68528-80-3) and Bis-PEG8-NHS ester.

In some aspects, the present disclosure provides a pharmaceutical composition comprising an antibody-oligonucleotide conjugate (AOC) comprising: VH complementarity determining regions (CDRs) having the amino acid sequences of NYGMH (SEQ ID NO:15); CDR H2.1: YISSGSSTIYYADTVKG (SEQ ID NO:4); CDR H3.1: RGLLLDY (SEQ ID NO:5), and VL CDRs having the amino acid sequences of RASKSVSTSSYSYMH (SEQ ID NO:9); CDR L2.1: YASYLES (SEQ ID NO:10); CDR L3.1: QHSREFPWT (SEQ ID NO:11), SPP (CAS: 341498-08-6), and a cMyc-siRNA sequence described herein (SEQ ID NOs: 140-151 and 240-265).

### A. Pharmaceutically Acceptable Carriers

The pharmaceutical composition can be in solution, emulsions, or in a suspension (for example, incorporated into microparticles, liposomes, or cells). An appropriate amount of a pharmaceutically-acceptable salt can be used in the formulation to render the formulation isotonic. Examples of pharmaceutically-acceptable carriers include, but are not limited to, saline, Ringer's solution and dextrose solution. The pH of the solution can be from about 5 to about 8, or from about 7 to about 7.5. Pharmaceutical compositions can include carriers, thickeners, diluents, buffers, preservatives, and surface-active agents. Further carriers include sustained release preparations such as semi-permeable matrices of solid hydrophobic polymers containing the antibody, which matrices are in the form of shaped particles, e.g., films, liposomes or microparticles. It will be apparent to those persons skilled in the art that certain carriers can be used, depending upon, for instance, the route of administration and concentration of composition being administered. Pharmaceutical compositions can also include one or more active ingredients such as antimicrobial agents, anti-inflammatory agents, and anesthetics.

Preparations for parenteral administration include sterile aqueous or non-aqueous solutions, suspensions, and emulsions. Parenteral vehicles include sodium chloride solution, Ringer's dextrose, dextrose and sodium chloride, lactated Ringer's, or fixed oils. Preservatives and other additives can also be present such as, for example, antimicrobials, anti-oxidants, chelating agents, and inert gases.

To aid dissolution of antibodies into the aqueous environment a surfactant might be added as a wetting agent. Surfactants can include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants could be present in the formulation of the protein or derivative either alone or as a mixture in different ratios. Additives which potentially enhance uptake of peptides are for instance the fatty acids oleic acid, linoleic acid and linolenic acid.

In certain aspects, the compositions disclosed herein are formulated with additional agents that promote entry into the desired cell or tissue. Such additional agents include micelles, liposomes, and dendrimers.

### B. Dosages

The term “effective amount” refers an amount that is non-toxic to a subject or a majority or normal cells, but is an amount of the active agent that is sufficient to provide a desired effect. This amount can vary from subject to subject, depending on the species, age, and physical condition of the subject, the severity of the disease that is being treated, the particular conjugate, or more specifically, the particular active agent used, its mode of administration, and the like.

The term “pharmaceutically acceptable” refers to the fact that the carrier, diluent or excipient must be compatible with the other ingredients of the formulation and not deleterious to the recipient thereof. For example, the carrier, diluent, or excipient or composition thereof can be administered to a subject along with an antibody-drug conjugate (AOC) of the disclosure without causing any undesirable biological effects or interacting in an undesirable manner with any of the other components of the pharmaceutical composition in which it is contained. Pharmaceutical compositions including the AOCs can be administered by any suitable means, for example, parenterally, such as by subcutaneous, intravenous, intramuscular, intrathecal, or intracisternal injection or infusion techniques (e.g., as sterile injectable aqueous or non-aqueous Solutions or Suspensions) in dosage formulations containing non-toxic, pharmaceutically acceptable vehicles or diluents. In some aspects the conjugate is administered parenterally, or intravenously.

The pharmaceutical compositions of an AOC described herein can be administered either alone or in combination with other therapeutic agents, can conveniently be presented in unit dose form and can be prepared by any of the methods well known in the art of pharmacy. All methods include bringing the conjugate into association with the carrier, which constitutes one or more accessory ingredients, are contemplated. Techniques for formulation and administration are generally known in the art. Suitable routes can be, for example, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, intrasternal, topical, epidermal, or mucosal administration.

### V. Methods of Use

In some aspects, the present disclosure provides methods of treating cancer. The compositions, conjugates, and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth.

The term “cancer” or “tumor” refers to a cell that displays uncontrolled growth, invasion upon adjacent tissues, and often metastasis to other locations of the body.

In some aspects, the present disclosure provides a method of treating cancer in a subject in need thereof, comprising administering a therapeutically effective amount of a pharmaceutical composition comprising an antibody-oligonucleotide conjugate (AOC) having the formula A-(L-Pᵣ)_{q}, wherein: A is a 3E10 antibody or antigen-binding fragment thereof, L is a linker, optionally wherein L is a cleavable linker, P is an oligonucleotide moiety as described herein, optionally wherein P is an siRNA molecule that targets cMyc mRNA, ris an integer from 1 to 4, and q is an integer from 1 to 16; and wherein the 3E10 antibody or antigen-binding fragment thereof comprises a heavy chain variable region (VH) CDR1 comprising the amino acid sequence of NYGMH (SEQ ID NO:15). In some aspects, the 3E10 antibody or antigen-binding fragment thereof comprises (a) a light chain variable region (VL) complementarity determining region (CDR) 1 comprising the amino acid sequence of RASKSVSTSSYSYMH (SEQ ID NO:9), (b) a VL CDR2 comprising the amino acid sequence of YASYLES (SEQ ID NO:10), and (c) a VL CDR3 comprising the amino acid sequence of QHSREFPWT (SEQ ID NO: 11), and (d) a heavy chain variable region (VH) CDR1 comprising the amino acid sequence of NYGMH (SEQ ID NO: 15), (e) a VH CDR2 comprising the amino acid sequence of YISSGSSTIYYADTVKG (SEQ ID NO: 4), and (f) a VH CDR3 comprising the amino acid sequence of RGLLLDY (SEQ ID NO: 5).

In some aspects, the 3E10 antibody or antigen-binding fragment thereof comprises VH complementarity determining regions (CDRs) having the amino acid sequences of NYGMH (SEQ ID NO:15); CDR H2.1: YISSGSSTIYYADTVKG (SEQ ID NO:4); CDR H3.1: RGLLLDY (SEQ ID NO:5), and VL CDRs having the amino acid sequences of RASKSVSTSSYSYMH (SEQ ID NO: 9); CDR L2.1: YASYLES (SEQ ID NO:10); CDR L3.1: QHSREFPWT (SEQ ID NO:11), and a pharmaceutically acceptable carrier. In some aspects, the present disclosure provides a method of inducing death of a cancer cell in a subject in need thereof, comprising administering a therapeutically effective amount of a pharmaceutical composition comprising an antibody-oligonucleotide conjugate (AOC), wherein: the 3E10 antibody or antigen-binding fragment thereof is conjugated through a cleavable linker to an siRNA molecule that targets cMyc mRNA; and wherein the 3E10 antibody or antigen-binding fragment thereof comprises a heavy chain variable region (VH) CDR1 comprising the amino acid sequence of NYGMH (SEQ ID NO:15), and a pharmaceutically acceptable carrier. In some aspects, the 3E10 antibody or antigen-binding fragment thereof comprises VH complementarity determining regions (CDRs) having the amino acid sequences of NYGMH (SEQ ID NO:15); CDR H2.1: YISSGSSTIYYADTVKG (SEQ ID NO: 4); CDR H3.1: RGLLLDY (SEQ ID NO:5), and VL CDRs having the amino acid sequences of RASKSVSTSSYSYMH (SEQ ID NO:9); CDR L2.1: YASYLES (SEQ ID NO:10); CDR L3.1: QHSREFPWT (SEQ ID NO:11), and a pharmaceutically acceptable carrier.

In some aspects, the 3E10 antibody or antigen-binding fragment thereof comprises a heavy chain variable region (VH) having an amino acid sequence that is identical to 3E10-HC_D31N (SEQ ID NO:13) or 3E10-VH_D31N (SEQ ID NO:14); and a light chain variable region (VL) having an amino acid sequence that is identical to 3E10-VL_D31N (SEQ ID NO:20) or 3E10-VL-VR_D31N (SEQ ID NO:21). In embodiments, the antibody or antigen-binding fragment thereof comprises a full length heavy chain (HC) comprising an amino acid sequence that is identical to SEQ ID NO:13.

In some aspects of the methods disclosed herein, the amino acid residue corresponding with N31 of the heavy chain CDR1 is substituted with R. In some aspects, the amino acid residue corresponding with N31 of the heavy chain CDR1 is substituted with L. In some aspects, the amino acid residue corresponding with N31 of the heavy chain CDR1 is substituted with K.

In some aspects of the methods disclosed herein, the antibody or antigen-binding fragment thereof is murine, chimeric, humanized, or human.

In some aspects of the methods disclosed herein, the pharmaceutical composition comprising an antibody-oligonucleotide conjugate (AOC) provided herein, is a cleavable linker. In some aspects, (L) the cleavable linker is an acid-labile linker, a protease-sensitive linker, a photo-labile linker, or a disulfide-containing linker. In some aspects, the cleavable linker is selected from the group consisting of SPDB (CAS: 115088-08-7) or SPP (CAS: 341498-08-6). In some aspects, the cleavable linker is a homo-bi-functional linker, optionally comprising an alkyl or polyethylene glycol (PEG) chain. In some aspects, the cleavable homo-bi-functional linker is selected from the group consisting of DSP (Lomant's Reagent) (CAS: 57757-57-0) and Acid-PEG4-S-S-PEG4-Acid (CAS: 2055015-40-0).

In some aspects of the methods disclosed herein, the pharmaceutical composition comprising an antibody-oligonucleotide conjugate (AOC) provided herein, is a non-cleavable linker. In some aspects, the non-cleavable linker is selected from DBCO-C6-NHS ester (CAS: 1384870-47-6), Az/DBCO-PEG 8 and DBCO-PEG8-NHS ester. In some aspects, the non-cleavable linker is a homo-bi-functional linker, optionally comprising an alkyl or polyethylene glycol (PEG) chain. In some aspects, the non-cleavable homo-bi-functional linker is selected from DSS (CAS: 68528-80-3) and Bis-PEG8-NHS ester.

In embodiments of the methods disclosed herein, L is a cleavable linker. In embodiments, L is selected from succinyl, O-succinyl, 4-succinimidyl-oxycarbonyl-a-(2-pyridyldithio) toluene, sulfosuccinimidyl 6-(3′-(2-pyridyldithio) propionamido) hexanoate, N-succinimidyl-3-(−2-pyridyldithio)-proprionate, succinimidyl 6-(3 (2-pyridyldithio) propionamido) hexanoate, 3-(2-pyridyldithio)-propionyl hydrazide, S-(2-thiopyridyl)-L-cysteine, N-succinimidyl 4-(2-pyridyldithio) butanoate (SPDB), N-succinimidyl 4-(2-pyridyldithio)-2-sulfobutanoate (sulfo-SPDB), N-succinimidyl 4-(2-pyridyldithio) pentanoate (SPP), CL2A, maleimidocaproyl-valine-citrulline-p-aminobenzoyloxycarbonyl (MC-vc-PAB) and N-succinimidyl 4-(2-pyridyldithio)-2-sulfopentanoate (sulfo-SPP).

In embodiments of the methods disclosed herein, L is a non-cleavable linker. In embodiments, L is selected from N-succinimidyl (4-iodoacetyl)-aminobenzoate, sulfosuccinimidyl (4-iodoacetyl)-aminobenzoate, dichlorotriazinic acid, N-succinimidyl-[(N-maleimidopropionamido)-tetraethyleneglycol]ester (NHS-PEG4-maleimide), N-succinimidyl 4-(maleimidomethyl)N-sulfosuccinimidyl cyclohexanecarboxylate (SMCC), or 4-(maleimidomethyl)cyclohexanecarboxylate (sulfoSMCC).

In embodiments, the present disclosure provides a method of treating cancer in a subject in need thereof, comprising administering a therapeutically effective amount of a pharmaceutical composition comprising an AOC disclosed herein that has the formula A-(L-Pᵣ)_{q}, wherein: A is a 3E10 antibody or antigen-binding fragment thereof, L is a linker, P is a payload as described herein, r is an integer from 1 to 4, and q is an integer from 1 to 16; wherein the 3E10 antibody or antigen-binding fragment thereof comprises a heavy chain variable region (VH) CDR1 comprising the amino acid sequence of XYGMH (SEQ ID NO: 16), wherein X=D, N, R, L, or K and corresponds to an amino acid residue at position 31 of the 3E10 heavy chain, and wherein D31 is wild-type 3E10 CDR1.

### A. Cancers

In some aspects, the subject has a cancer selected from colorectal cancer, pancreatic cancer, lung cancer, ovarian cancer, liver cancer, breast cancer, brain cancer, kidney cancer, prostate cancer, gastrointestinal cancer, melanoma, cervical cancer, bladder cancer, glioblastoma, glioma, brain stem glioma, head and neck cancer, lymphomas, Hodgkin lymphoma, Non-Hodgkin lymphoma, cutaneous B-cell lymphoma, cutaneous T-cell lymphoma, Waldenstrom macroglobulinemia, chronic lymphocytic leukemia, leukemia, neuroblastoma, Wilms tumor, bone cancer, brain stem tumor, childhood diffuse intrinsic pontine glioma (DIPG), retinoblastoma, rhabdoid tumor, sarcoma, spinal cord tumor, endocrine cancer, esophageal cancer, gastric cancer, hepatobiliary cancer, myeloma, renal cancer, thyroid cancer, uterine cancer, carcinoma, blastoma, papilloma, adenoma, an astrocytic tumor, an oligodendroglial tumor, an oligoastrocytic tumor, an ependymal tumor, a choroid plexus tumor, a neuronal or mixed neuronal-glial tumor, tumor of the pineal region, embryonal tumor, or an otherwise uncategorized neuroepithelial tumor.

Examples of cancer can also include small-cell lung cancer (SCLC), non-small cell lung cancer (NSCLC), squamous NSCLC, nonsquamous NSCLC, clear cell carcinoma, endometrial cancer, renal cell carcinoma (RCC), hormone refractory prostate adenocarcinoma, glioblastoma multiforme, stomach cancer, hepatoma (hepatocellular carcinoma), colon carcinoma, head and neck squamous cell carcinoma (HNSCC), germ cell tumor, pediatric sarcoma, sinonasal natural killer, metastatic malignant melanoma, cutaneous or intraocular malignant melanoma, mesothelioma, skin cancer, cancer of the anal region, testicular cancer, carcinoma of the fallopian tubes, carcinoma of the endometrium, carcinoma of the cervix, carcinoma of the vagina, carcinoma of the vulva, cancer of the small intestine, cancer of the parathyroid gland, cancer of the adrenal gland, sarcoma of soft tissue, cancer of the urethra, cancer of the penis, cancer of the ureter, carcinoma of the renal pelvis, neoplasm of the central nervous system (CNS), spinal axis tumor, pituitary adenoma, Kaposi's sarcoma, epidermoid cancer, squamous cell cancer, environmentally-induced cancers including those induced by asbestos, virus-related cancers or cancers of viral origin, human papilloma virus (HPV)-related or -originating tumors, and any combinations of said cancers.

Examples of cancer can also include acute leukemia (ALL), acute myelogenous leukemia (AML), chronic myelogenous leukemia (CML), undifferentiated AML, myeloblastic leukemia, promyelocytic leukemia, myelomonocytic leukemia, monocytic leukemia, erythroleukemia, megakaryoblastic leukemia, isolated granulocytic sarcoma, chloroma B-cell lymphoma, T-cell lymphoma, lymphoplasmacytoid lymphoma, monocytoid B-cell lymphoma, mucosa-associated lymphoid tissue (MALT) lymphoma, anaplastic large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic lymphoma, peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, lymphoplasmacytoid lymphoma (LPL) with Waldenstrom's macroglobulinemia, myeloma, IgG myeloma, light chain myeloma, nonsecretory myeloma, smoldering myeloma (indolent myeloma), solitary plasmocytoma, multiple myeloma, hairy cell lymphoma, and any combinations of said cancers.

Malignant tumors which can be treated can be classified according to the embryonic origin of the tissue from which the tumor is derived. Carcinomas are tumors arising from endodermal or ectodermal tissues such as skin or the epithelial lining of internal organs and glands. The disclosed compositions can be effective in treating carcinomas. Sarcoma, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors can show up at numerous organs or tissues of the body to establish a cancer.

In some aspects of the methods of the present disclosure, the administration of the AOC induces cell killing that is measurable by a tumor cell-killing potency (EC50) value. In some aspects, the administration of the AOC induces cell killing in a breast cancer cell line with an EC50 value of from about 0.2-13 nM. In some aspects, the administration of the AOC induces cell killing in a colorectal cancer cell line with an ECso value of from about 50-90 nM. In some aspects, the administration of the AOC induces cell killing in a colorectal cancer cell line with an EC50 value of from about 1-3 nM. In some aspects, the administration of the AOC induces cell killing in an ovarian cancer cell line with an EC50 value of from about 2-20 nM. In some aspects, the administration of the AOC induces cell killing with an EC50 value of from about 2.5-9.5 nM. In some aspects, the administration of the AOC induces cell killing with an EC50 value of from about 50-90 nM.

In some embodiments, the cancer is a carcinoma, a sarcoma, a blastoma, a papilloma, or an adenoma. In some embodiments, the cancer is metastatic cancer.

In some embodiments, the cancer is selected from the group consisting of bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, melanoma, myeloma, ovarian cancer, pancreatic cancer, prostate cancer, renal cancer, thyroid cancer, and uterine cancer.

In some embodiments, the cancer is lung cancer. In some embodiments, the lung cancer is small-cell lung cancer (SCLC), non-small cell lung cancer (NSCLC), squamous NSCLC, or nonsquamous NSCLC. In some embodiments, the lung cancer is non-small cell lung cancer (NSCLC).

In some embodiments, the cancer is breast cancer. In some embodiments, the cancer is BRCA2 negative breast cancer. In some embodiments, the cancer is triple negative breast cancer.

In some embodiments, the cancer is a skin cancer selected from the group consisting of basal cell carcinoma, squamous cell carcinoma, and melanoma. In some embodiments, the cancer is melanoma.

In some embodiments, the cancer is a cancer of the central nervous system. In some embodiments, the cancer is a neuroepithelial brain or spinal tumor selected from the group consisting of a medulloblastoma, an astrocytic tumor, an oligodendroglial tumor, an oligoastrocytic tumor, an ependymal tumor, a choroid plexus tumor, a neuronal or mixed neuronal-glial tumor, a tumor of the pineal region, an embryonal tumor, or an otherwise uncategorized neuroepithelial tumor.

In some embodiments, the administering is by parenteral administration. In some embodiments, the parenteral administration is intramuscular administration, intravenous administration, or subcutaneous administration.

### B. Dosages and Administration

Administration of the suitable compositions provided herein can be effected by different ways, e.g., by intravenous, intraperitoneal, subcutaneous, intramuscular, intrathecal, intratumoral, intraocular, intravitreal, intraarterial, topical, intradermal, etc., administration. The route of administration, of course, depends, inter alia, on the AOC contained in the pharmaceutical composition.

The dosage regimen will be determined by the attending physician and other clinical factors. As is well known in the medical arts, dosages for any one patient depends on many factors, including the patient's size, body surface area, age, sex, the particular compound to be administered, time and route of administration, the kind and stage of infection or disease, general health and other drugs being administered concurrently.

### C. Uses

Uses of an AOC described herein are also contemplated. In some aspects, the present disclosure provides a use of an AOC described herein, or a pharmaceutical composition comprising an AOC described herein, for the treatment of cancer in a subject in need thereof. In some aspects, the present disclosure provides a use of AOCs described herein, or the pharmaceutical composition comprising an AOC described herein, for inducing death of a cancer cell in a subject in need thereof. In some aspects, the present disclosure provides a use of an AOC described herein, or the pharmaceutical composition comprising an AOC described herein, for inhibiting tumor growth in a subject in need thereof.

### EXAMPLES

### Example 1: Assessing In Vitro Myc Luciferase Activity in the HCT116 Cell Line Treated with 3E10-Myc-siRNA

Several 3E10-cMyc siRNA conjugates were evaluated in the human colon cancer cell line, HCT116, for the inhibition of the expression of Myc luciferase reporter. The HCT116 cell line contains mutated B-catenin, which leads to the accumulation of β-catenin and constitutive activation of downstream Myc that induces the expression of Myc luciferase reporter (HCY116 Myc-Luc cells).

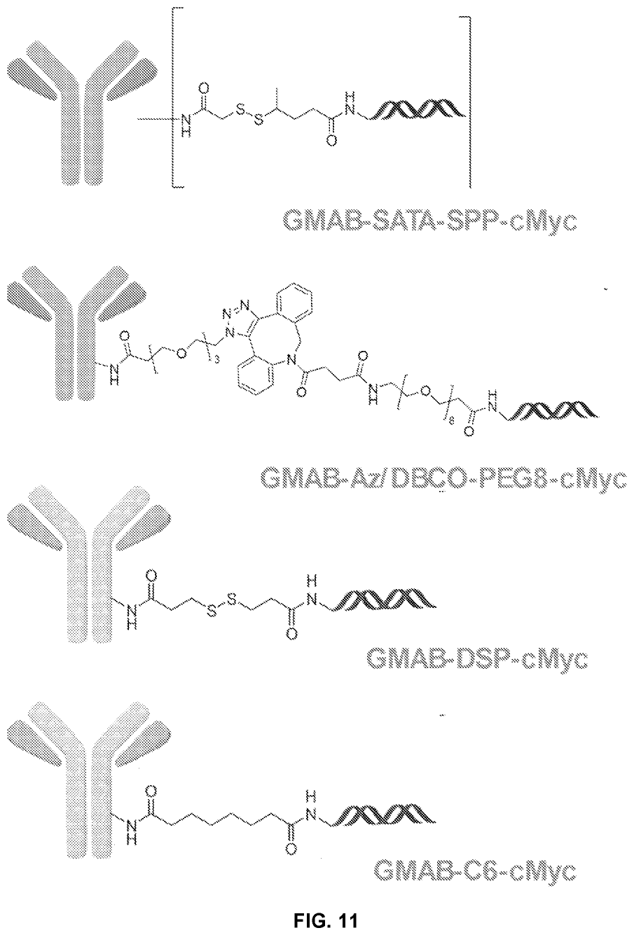

3E10-cMyc siRNA conjugates were made by conjugating the Myc siRNA shown in Table 3 to a humanized 3E10 antibody (V66) using a cleavable or non-cleavable linker as shown in FIG. 11 (3E10-SATA-SPP-cMyc, 3E10-Az/DBCO-PEG8-cMyc, 3E10-DSP-cMyc, and 3E10-C6-cMyc. The 3E10-cMyc siRNA conjugates were added at concentrations of 2.5 μM or 5 μM to wells with approximately 20,000 cells HCT116 Myc-Luc cells. Cells were treated with the humanized 3E10 antibody (V66) alone, or PBS to serve as controls. Approximately 48 hours after treatment, luciferase expression levels were measured. Results shown as percentage of Relative Light Units (RLU) compared to untreated cells, and are are normalized to PBS treated control wells.

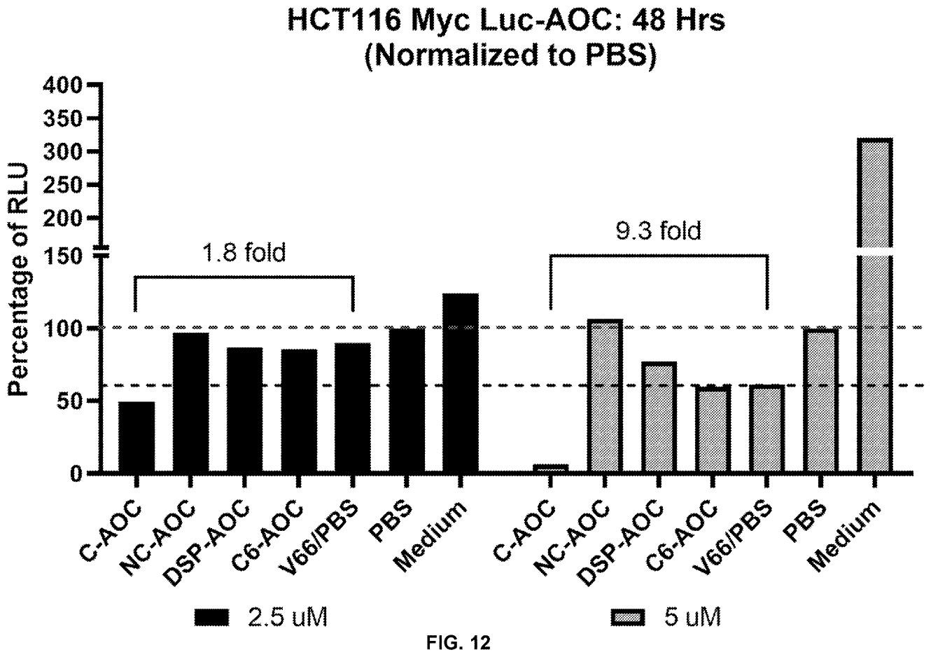

FIG. 12 shows the results of Myc luciferase activity in HCT1116 cells treated with 3E10-SATA-SPP-cMyc, 3E10-Az/DBCO-PEG8-cMyc, 3E10-DSP-cMyc, and 3E10-C6-cMyc. Among these four 3E10-cMyc-siRNA conjugates, 3E10-SATA-SPP-cMyc, with a cleavable linker and designated as “C-AOC” in FIG. 12, showed the most significant reduction in luciferase expression in the Myc Luc reporter assay.

The cMyc-siRNA exemplified in the four 3E10-cMyc-siRNA conjugates, 3E10-SATA-SPP-cMyc, 3E10-Az/DBCO-PEG8-cMyc, 3E10-DSP-cMyc, and 3E10-C6-cMyc, is listed in Table 3.

TABLE 3
cMyc-siRNA sequence

| | |
|---|---|
| 5′-N6-G-mC-U-U-mU-U-mU-U-G-C-mC-mC-U-mG-C-G-mU-mG-A-C- | SEQ ID NO: 144 |
| C-A-G-dA-dT-3′ (sense) | |
| 3′-mC-mU-mC-G-mA-A-A-A-A-A-C-G-G-G-mA-C-mG-C-A-C-U-G-G- | SEQ ID NO: 147 |
| mU-mC-mU-mA-5′ (antisense) | |

In Table 3, Deoxyribonucleotides are indicated as dA, dC, dG and dT. 2′-O-methyl-modified nucleotides are denoted as mA, mC, mG and mU. The phosphodiester linkages are indicated as (-). The 5′-aminohexyl moiety on the sense strand of the duplex siRNA for conjugation to antibody is denoted as N6.

Other exemplary cMyc-siRNA sequences are shown in Tables 4-5.

TABLE 4
Exemplary cMyc-siRNA sequence

| | |
|---|---|
| 5′ N6-G-C-C-A-C-A-G-C-A-U-A-C-A-U-C-C-U-G-U-U-U-3′ (sense) | SEQ ID NO: 145 |
| 3′-U-U-C-G-G-U-G-U-C-G-U-A-U-G-U-A-G-G-A-C-A-5′ (antisense) | SEQ ID NO: 148 |

In Table 4, the phosphodiester linkages are indicated as (-) and the 5′-aminohexyl moiety on the sense strand of the duplex siRNA for conjugation to antibody is denoted as N6.

TABLE 5
Exemplary cMyc-siRNA sequence

| | |
|---|---|
| 5′-N6*2′FG*mC*2′FC-mA-2′FC-mA-2′FG-mC-2′FA-mU-2′FA-mC- | SEQ ID NO: 146 |
| 2′FA-mU-2′FC-mC-2′FU-mG-2′FU-mU-2′FU-3′ (sense) | |
| 3′-mU*2′FU*mC-2′FG-mG-2′FU-mG-2′FU-mC-2′FG-mU-2′FA-mU- | SEQ ID NO: 149 |
| 2′FG-mU-2′FA-mG-2′FG-mA*2′FC*mA-5′ (antisense) | |

In Table 5, the 2′-O-methyl-modified nucleotides are denoted as mA, mC, mG and mU. The 2′-fluoro-modified nucleotides are denoted as 2′FA, 2′FC, 2′FG. The 2′FU phosphorothioate linkages are indicated as (*). The phosphodiester linkages are indicated as (-). The 5′-aminohexyl moiety on the sense strand of the duplex siRNA for conjugation to antibody is denoted as N6.

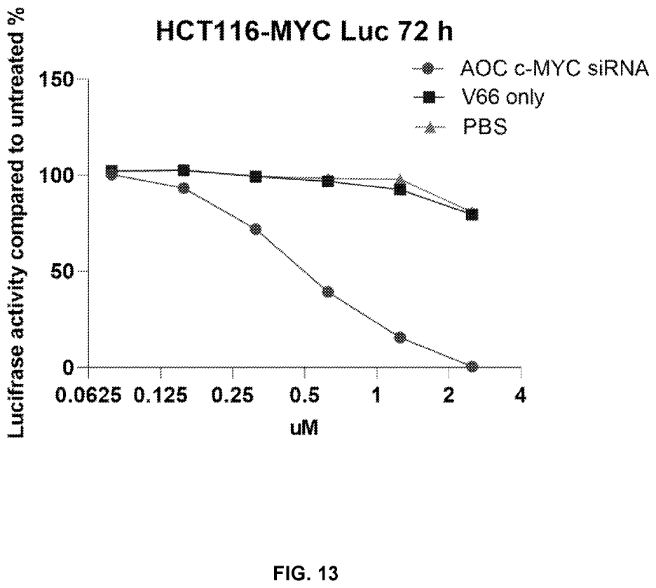

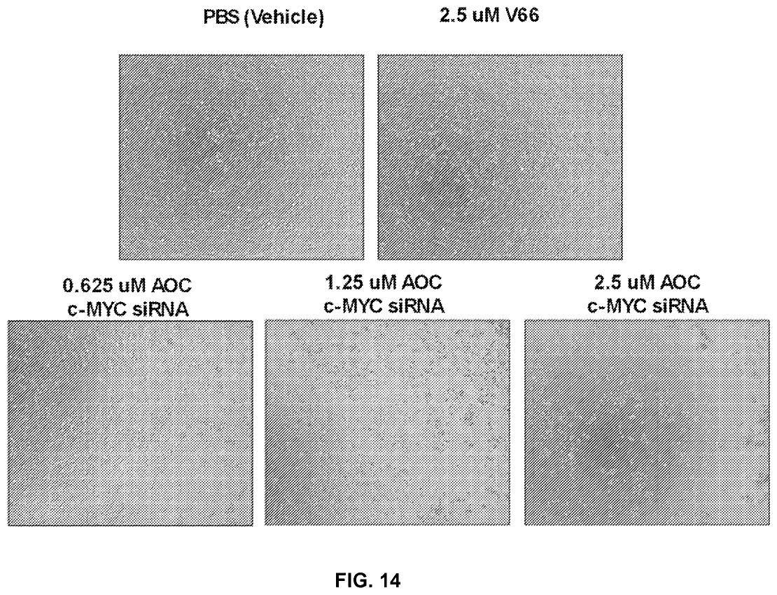

HCT116 Myc-Luc cells were treated with the 3E10-cMyc siRNA conjugate with a cleavable linker (i.e., 3E10-SATA-SPP-cMyc) in concentrations ranging from 0 mM to greater than 2 μM. Cells treated with the humanized 3E10 antibody (V66) alone, or PBS served as controls. Cells were harvested and luciferase activity was evaluated 72 hours after treatment. The 3E10-cMyc siRNA conjugate showed a concentration dependent reduction of luciferase activity at all concentrations relative to the humanized 3E10 antibody (V66) and PBS controls, especially at the maximum concentration of greater than 2 mM (~2.5 mM), as quantified in FIG. 13 and as shown in the micrographs in FIG. 14.

### Example 2: Dose Response Curves Measuring In Vitro Myc Luciferase Activity in a HCT116 Cell Line Treated with 3E10-Myc-siRNA

The effect of the 3E10-cMyc siRNA conjugate (i.e., 3E10-SATA-SPP-cMyc) was evaluated in the human colon cancer cell line, HCT116, for the inhibition of the expression of Myc luciferase reporter. The HCT116 cell line contains mutated B-catenin, which leads to the accumulation of β-catenin and constitutive activation of downstream Myc that induces the expression of Myc luciferase reporter (HCT116 MYC-Luc).

A 3E10-cMyc siRNA conjugate with a cleavable linker was tested in duplicate. In each of the experiments, the 3E10-cMyc siRNA conjugates were added at the required concentration in 100 μL, followed by 2-fold serial dilutions in complete medium (50 μL). Approximately 20,000 cells in 50 μL complete medium were added to wells with HCT116 Myc Luc cells. Approximately 48 hours later, equal amount of Luciferase reagent added and read on plate reader. Units are normalized to untreated control wells, which is considered as 100%. Treatment with the humanized 3E10 antibody alone, designated as V66, or PBS served as controls for each of the experiments.

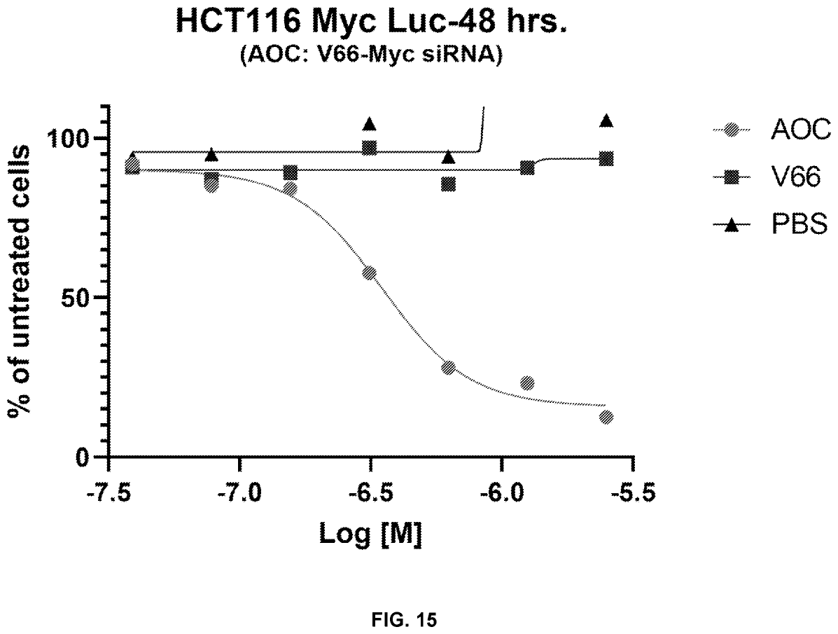

FIG. 15 shows the results of Myc luciferase activity in HCT116 Myc-Luc cells treated with a 3E10-cMyc siRNA conjugate with a cleavable linker, a humanized 3E10 antibody, and PBS. FIG. 15A shows the luciferase activity of the cells after treatment in Relative Light Units (RLU) and FIG. 15B shows the luciferase activity of the cells after treatment as a percentage (%) of untreated cells. The 3E10-cMyc siRNA conjugate with a cleavable linker showed significant luciferase activity compared to the controls in the Myc-Luc reporter assay, in a dose-dependent manner, especially at the maximum concentration of 2.5 mM.

### Example 3: In Vitro Cell Viability in OVCAR8 Cell Line Treated with 3E10-Myc-siRNA in a Dose-Dependent Manner

3E10-cMyc siRNA conjugates were evaluated in a dose-dependent manner in the human ovarian cancer cell line, OVCAR8, for the inhibition of cell viability using Cell Titer Glo assay that measures cellular ATP levels.

A 3E10-cMyc siRNA conjugate with a cleavable linker (i.e., 3E10-SATA-SPP-cMyc) were tested in in OVCAR8 cell viability assay. In the experiment 3E10-cMyc siRNA conjugates were at the required concentration in 100 μL, followed by 2-fold serial dilutions in complete medium (50 μL). Approximately 5,000 cells in 50 μL complete medium were added to wells with OVCAR8 cells. Approximately 72 hours later, equal amount of Cell Titer Glo reagent was added and read on plate reader. Units are normalized to untreated control wells, which is considered as 100%. Treatment with the humanized 3E10 antibody alone, designated as V66, or PBS served as controls for each of the experiments.

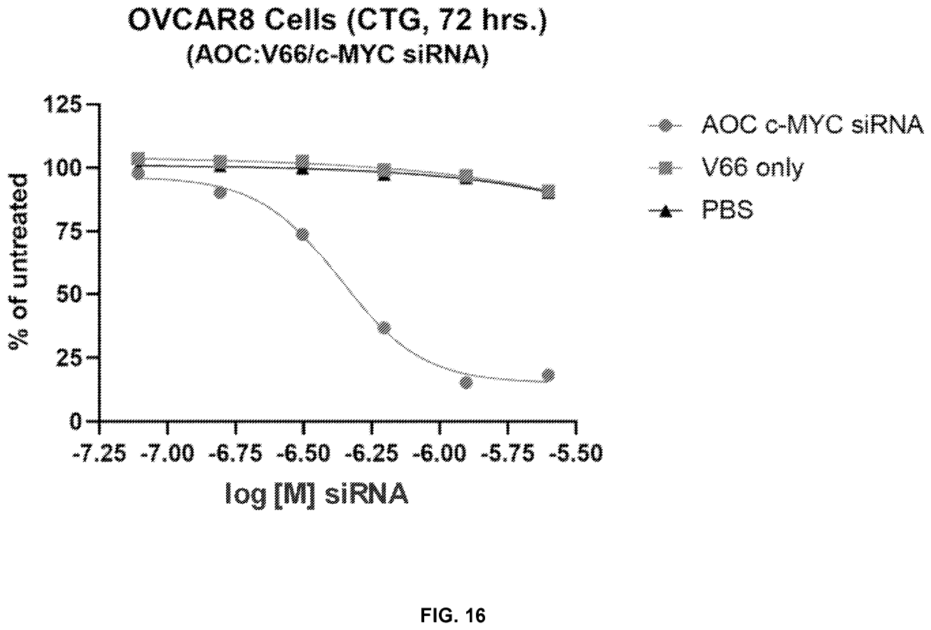

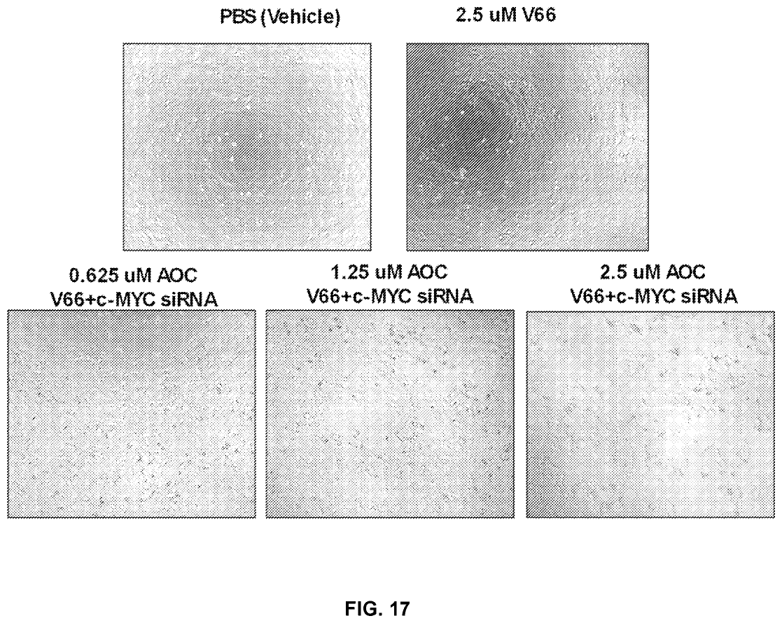

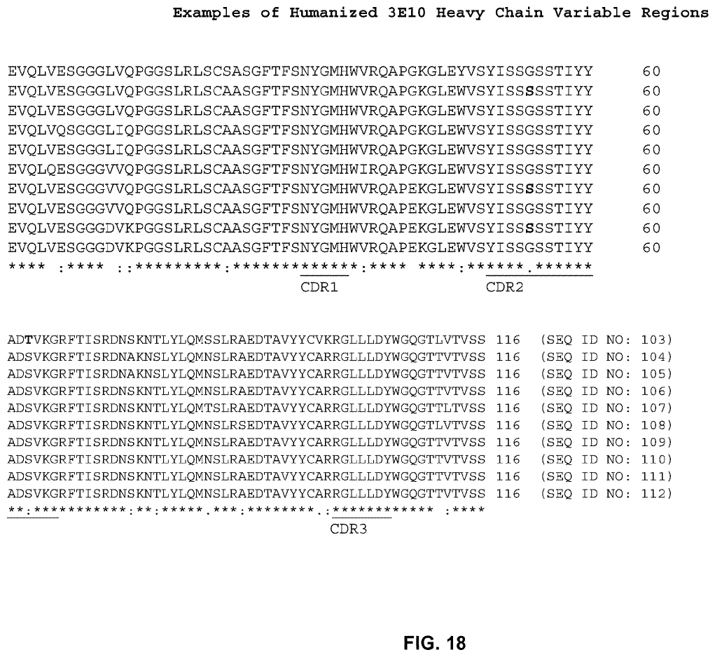

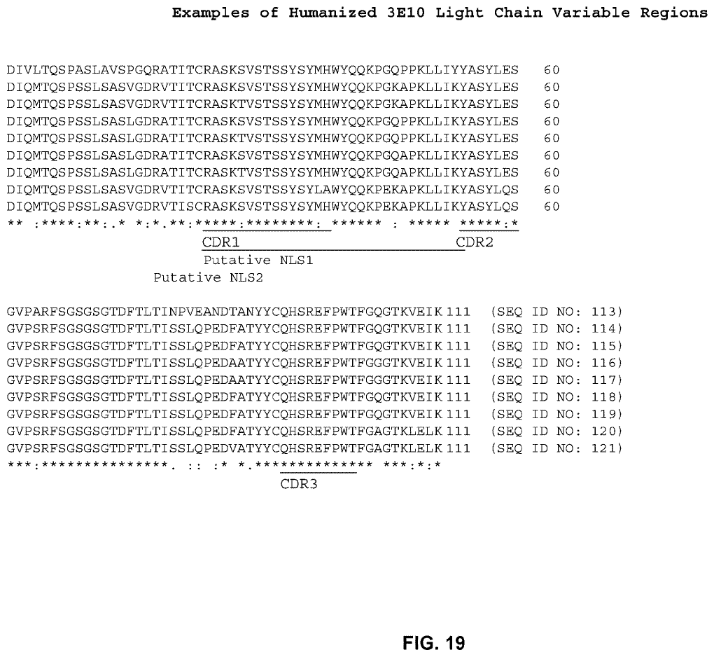

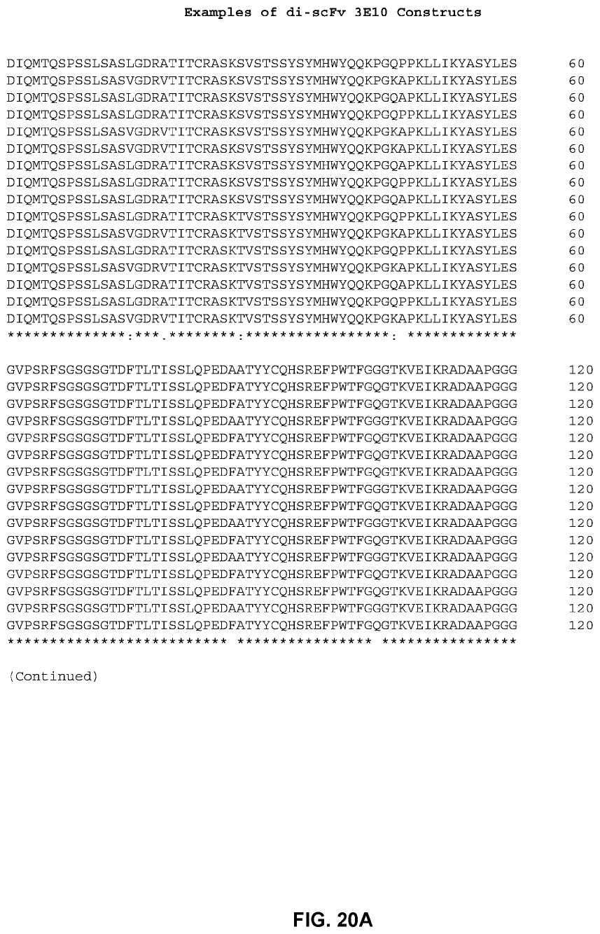

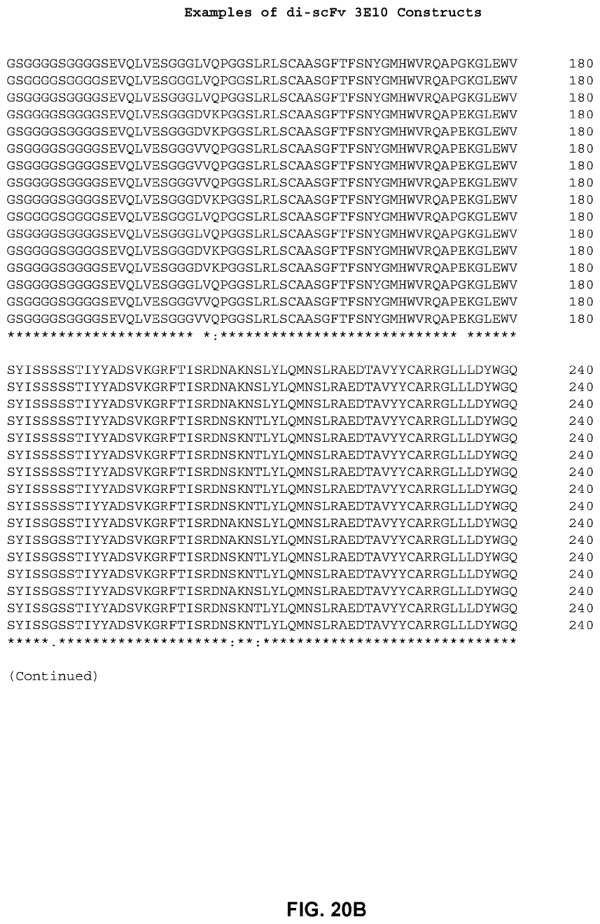

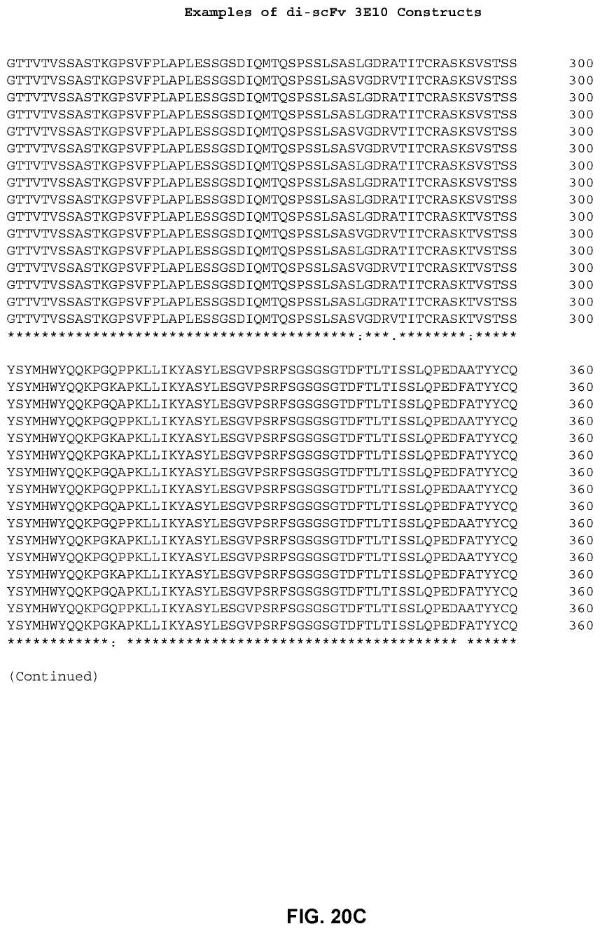

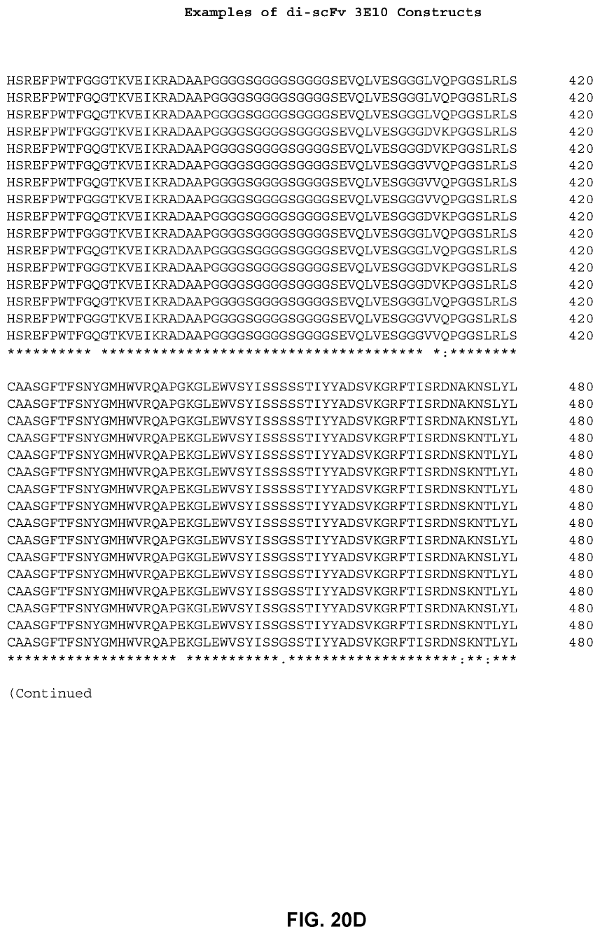

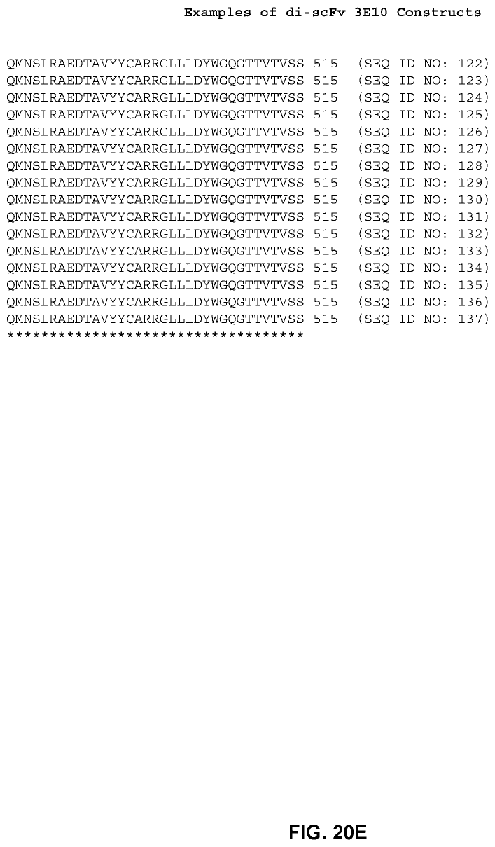

FIGS. 16-17 show the results of the Cell Titer Glo assay in OVCAR8 cells treated with the 3E10-cMyc siRNA conjugate with a cleavable linker, the humanized antibody (V66) alone, or PBS (vehicle). Only the 3E10-cMyc siRNA conjugate with a cleavable linker showed significant inhibition in cell viability assay at all concentrations relative to humanized 3E10 antibody (V66) and siRNA, in a dose dependent manner.

### Example 4: Effect of 3E10-D31N (V66) and Carboplatin Combination Treatment on Tumor Volume in a Xenograft OVCAR8 Ovarian Tumor Model

A humanized 3E10-D31N monoclonal antibody (V66) was evaluated in an xenograft OVCAR-8 ovarian tumor model as a single agent, in a non-covalent complex with a c-Myc antisense oligonucleotide (ASO), and in combination with the chemotherapeutic, carboplatin.

In the experiment, V66 was mixed with a c-Myc ASO at a 3:1 molar ratio of antibody to c-myc ASO for 30 minutes at room temperature with light agitation in PBS. V66 and c-Myc ASO concentrations in the complex were determined by methods described by Porterfield et. al. Virology. 2010). The Myc ASO targeted a c-Myc exon 2 mRNA and contained the following sequence: 5′-mU*mU*mC*A*C*C*A*T*C*T*C*mU*mC-3′ (SEQ ID NO:153). The 2′-O-methyl-modified ribose nucleotides are denoted as mA, mC, mG and mU. The Phosphorothioate linkages are indicated as (*). The deoxyribonucleotides are indicated as A, C, G and T.

In the experiment, mean tumor volume (±SEM mm³) was measured in a xenograft OVCAR-8 ovarian tumor model after administering each test agent according to the following groups: Vehicle Control (0 mg/kg) (Group 1), V66/scramble or nontargeting ASO (srASO) (90 mg/kg) (IV) (Group 2), Myc-ASO (3 mg/kg) (IV) (Group 3), V66/Myc-ASO (90 mg/kg) (IV) (Group 4), Carboplatin (70 mg/kg (IV) (IP) (Group 5), and V66/Myc-ASO (90 mg/kg) (IV)+Carboplatin (70 mg/kg) (IP) (Group 6). In Groups 1-6, the test agent was administered intravenously, with dosing every two days for 12 days (Q2D×12). For Groups 5 and 6, the Carboplatin was administered intraperitoneally, with weekly dosing for 4 weeks.

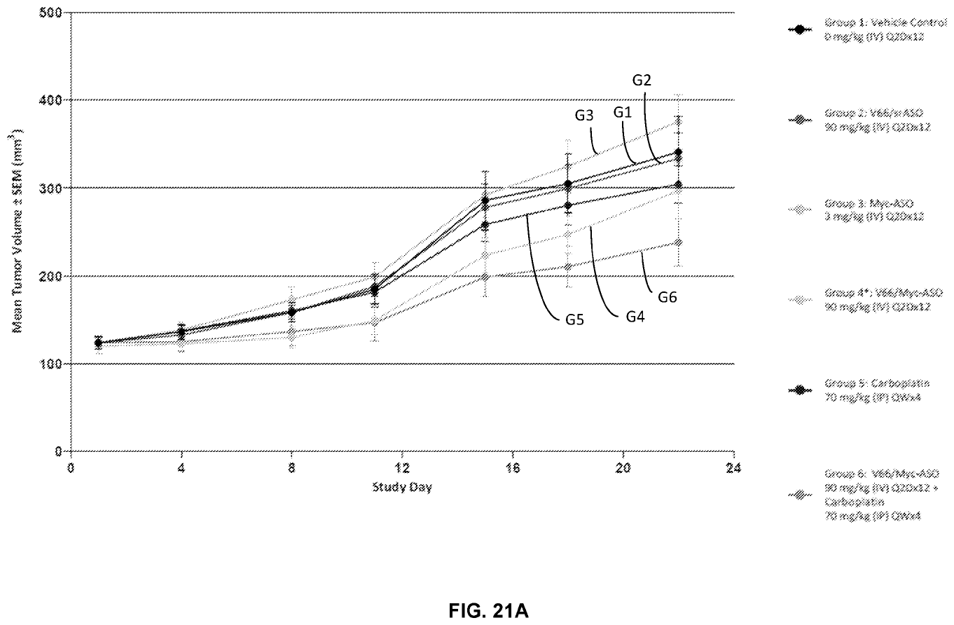

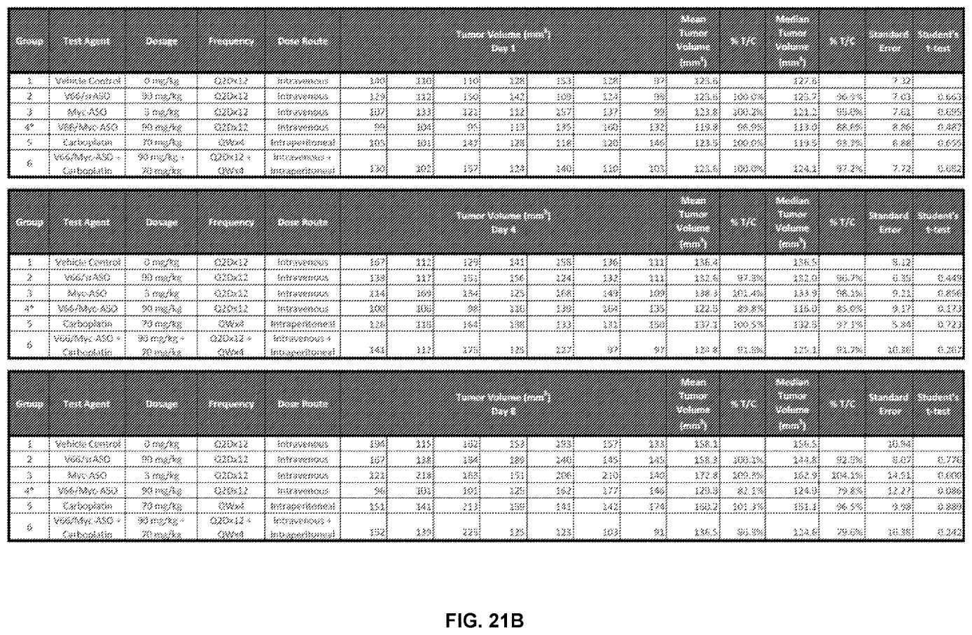

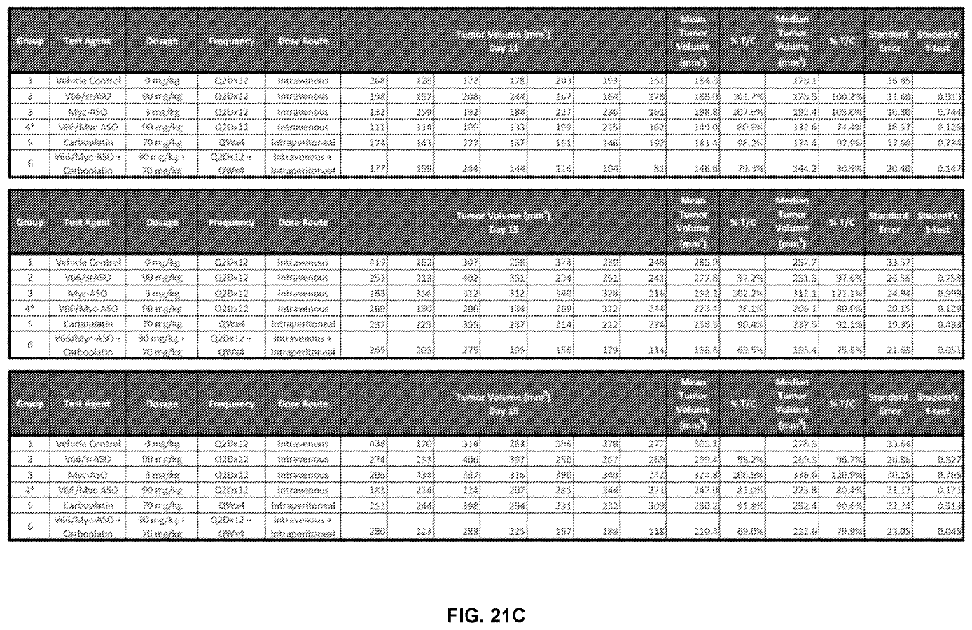

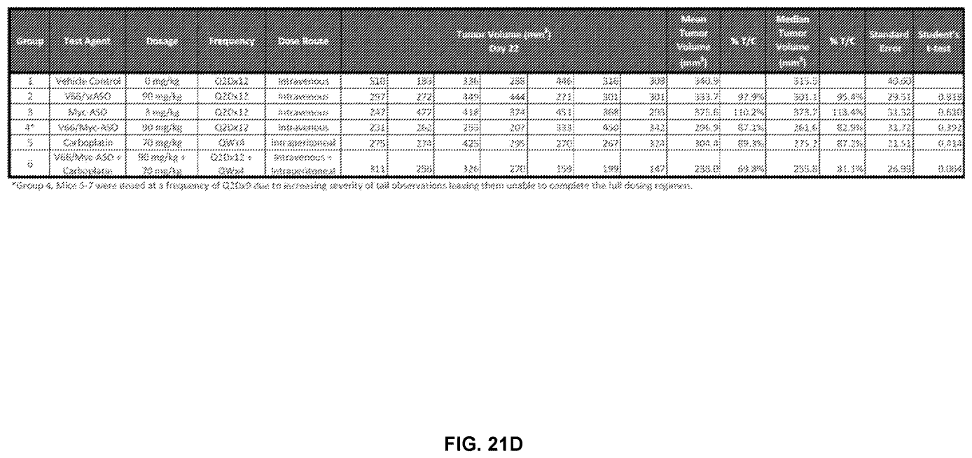

FIG. 21A shows the results of treatment with test agents (Groups 1-6) on tumor volume after a 24-day study period. At 22 days, Group 6: V66/Myc-ASO (90 mg/kg) (IV)+Carboplatin (70 mg/kg) resulted in a mean tumor volume of approximately 230 mm³. This differed from the administration of test agents in Group 4: V66/Myc-ASO (90 mg/kg) (IV) or Group 5: Carboplatin (70 mg/kg) which resulted in a mean tumor volume of approximately 300 mm³. This suggests that the combination treatment of Group 6 is more effective than the single agent treatments of Groups 4 and 5. Importantly, the control Groups 1-3 all exhibited mean tumor volumes of greater than 300 mm³ at day 22.

FIGS. 21B-21D show a data table for tumor volumes (mm³) for all 6 groups. The reduction in tumor volume on Day 18 is significant comparing Group 6: V66/Myc-ASO (90 mg/kg) (IV)+Carboplatin (70 mg/kg) to Group 1: Vehicle Control (0 mg/kg) according to Student's t-test (0.045).

### Example 5: Tryptic Peptide Mapping to Identify Preferential Lysine Residues for 3E10-D31N Monoclonal Antibody (V66) Conjugation

Since the variable regions and CDRs of the 3E10-D31N monoclonal antibody (V66) contain multiple lysine residues, it was investigated whether conjugation in this region may have a negative impact on antibody immunoreactivity and function.

To test this hypothesis, 3E10-D31N monoclonal antibody (V66) Lys-azide conjugation intermediates were digested and mapped by mass spectrometry to characterize modified lysines in the VH and VL chains of the antibody and as shown in FIGS. 22A and 22B. As is shown in FIG. 22B, most levels of lysine modification are modest and many of the modified lysines are not implicated in antibody function. As shown in FIG. 22C, the light chain K53 is the most modified lysine (nearly ⅔ instances) and is in the CDR2. These results indicate that any modification of VL K53 would negatively impact the ability of the 3E10-D31N humanized antibody (V66) to bind with with nucleic acids (e.g., DNA and/or RNA) and in turn have a negative impact on the function of an antibody-oligonucleotide conjugate (AOC).

### Example 6. Improved Cellular Internalization of 3E10-D31N Monoclonal Antibody (V66) Oligonucleotide Conjugates Utilizing Transglutaminase-Mediated Enzymatic Conjugation

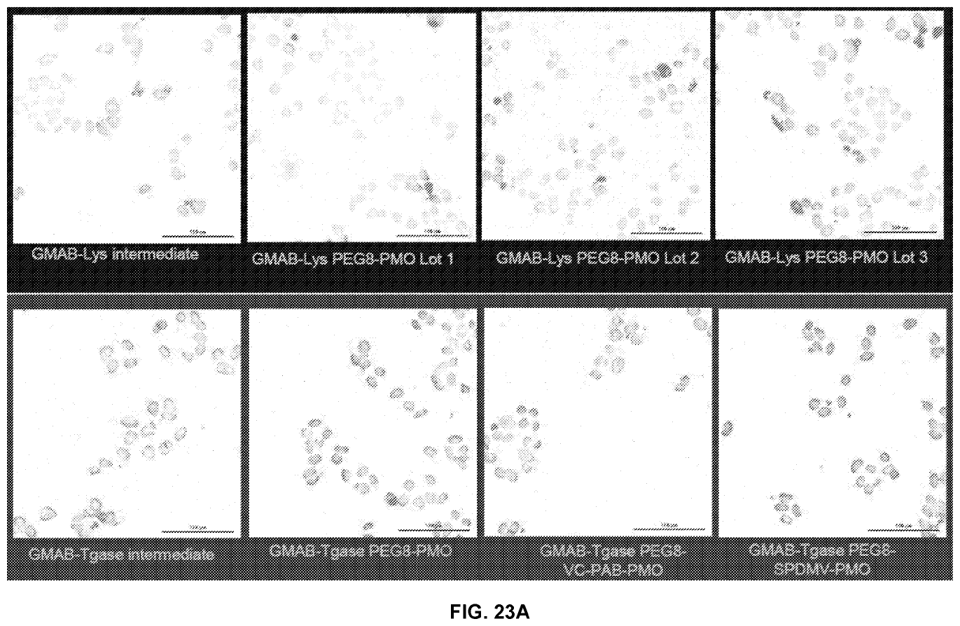

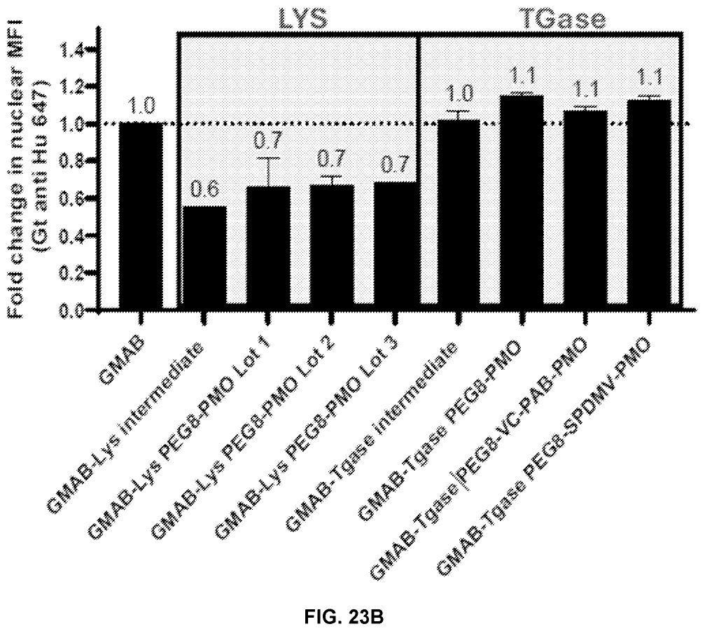

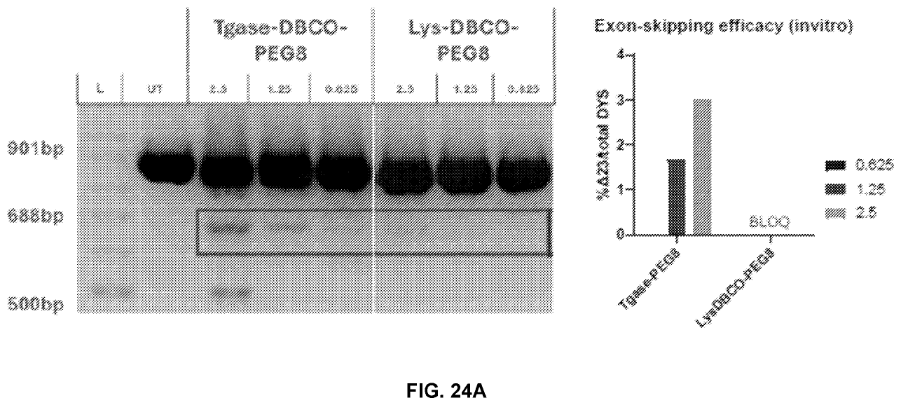

Internalization of 3E10-D31N monoclonal antibody (V66) oligonucleotide conjugates was investigated in a lung carcinoma epithelial cell line (A427 cells) comparing lysine versus transglutaminase-mediated enzymatic conjugation methods. As is shown in FIG. 23A, immunohistochemistry staining suggests 3E10-D31N monoclonal antibody (V66) oligonucleotide conjugates utilizing transglutaminase-mediated enzymatic conjugation show greater uptake than 3E10-D31N monoclonal antibody (V66) oligonucleotide conjugated with lysine intermediate. These results are further exemplified in FIG. 23B, which show fold change in nuclei median fluorescence intensity (MFI, a readout for antibody internalization) of all tested antibody oligonucleotide conjugate lots.

### Example 7. Modifications to 3E10-D31N Monoclonal Antibody (V66) Oligonucleotide Conjugates Effect Greater Exon Skipping in C2C12 Myotubules

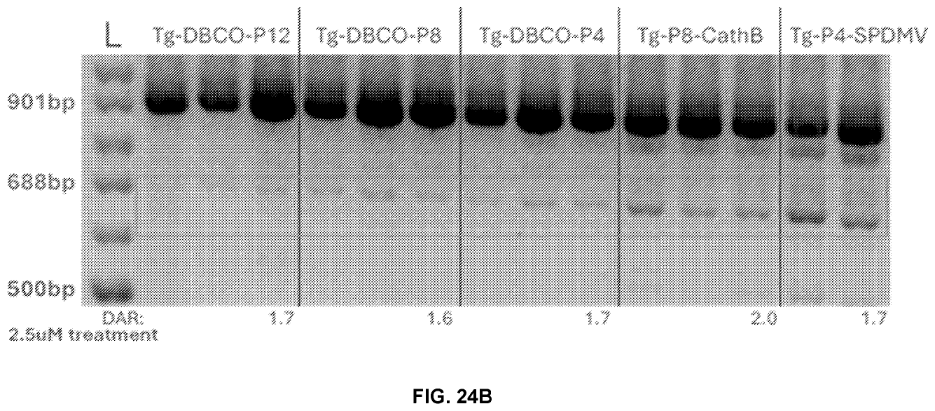

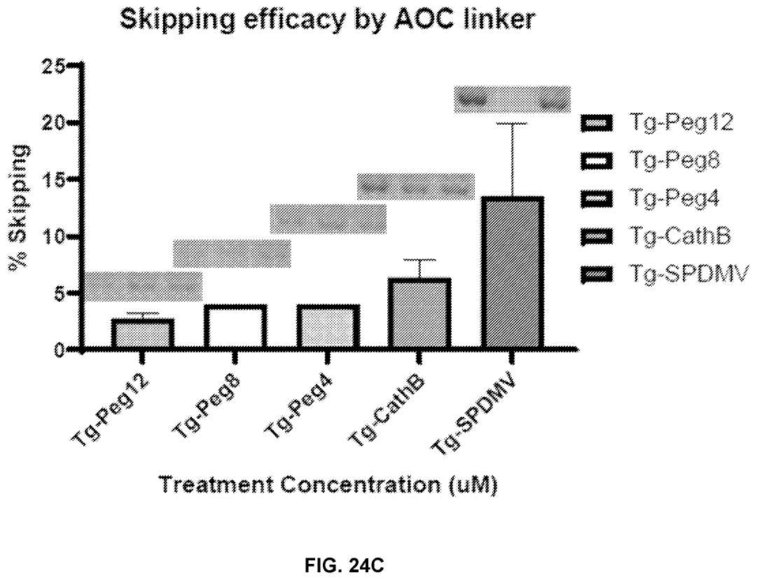

Lysine versus transglutaminase-mediated enzymatic conjugation methods were evaluated in 3E10-D31N monoclonal antibody (V66)-oligonucleotide conjugates (AOCs) to determine the effect on exon skipping in C2C12 myotubules. As is shown in FIG. 24A, transglutaminase-mediated enzymatic conjugation demonstrated improved exon skipping over lysine conjugation. Furthermore, as is shown in FIG. 24B, varying the length of the PEG linkers (e.g., PEG4, PEG8, and PEG12) did not significantly impact exon skipping. Lastly, as shown in FIG. 24C, the cleavable linkers, protease Cathepsin-B and SPDMV (disulfide), show enhanced exon-skipping compared to non-cleavable linkers with transglutaminase-mediated enzymatic conjugation. Furthermore, the hindered disulfide linker (SPDMV) conjugate (Tg-SPDMV) imparted the highest exon-skipping (~15%), representing greater than 10× improvement over lysine-PEG8 AOC (via lysine conjugation).

### Example 8. Linker Stability of 3E10-D31N Monoclonal Antibody (V66)-Oligonucleotide Conjugates (AOCs) in Mouse Serum

Linker stability was evaluated for several 3E10-D31N monoclonal antibody (V66)-oligonucleotide conjugates (AOCs) with a phosphorodiamidate morpholino (PMO) designed to improve the therapeutic efficacy targeting Duchenne muscular dystrophy (DMD) in mouse serum. The AOCs tested were: (1) DMD 3E10-D31N monoclonal antibody (V66)-SPP-PMO using lysine conjugation;

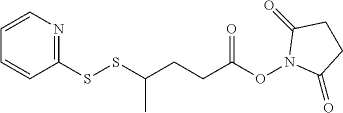 [SMILES: CC(CCC(=O)ON1C(=O)CCC1=O)SSc1ccccn1]

### SPP Linker

(2) DMD 3E10-D31N monoclonal antibody (V66)-Az-DBCO-PEG8-VC-PAB-PMO;

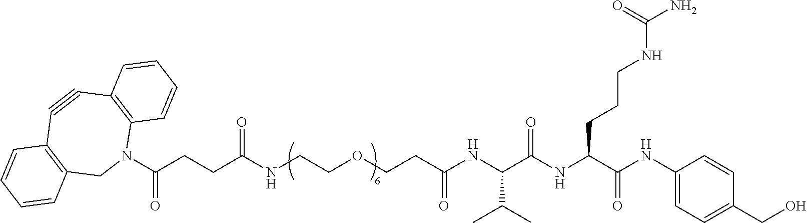

### DBCO-PEG8-VC-PAB Linker

(3) 3E10-D31N monoclonal antibody (V66) Az-DBCO-PEG8-PMO using transglutaminase (TGase) mediated enzymatic conjugation;

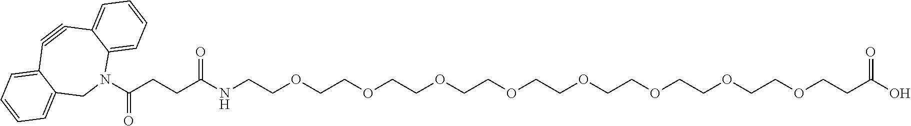 [SMILES: O=C(O)CCOCCOCCOCCOCCOCCOCCOCCOCCNC(=O)CCC(=O)N1Cc2ccccc2C#Cc2ccccc21]

### DBCO-PEG8 Linker

and (4) 3E10-D31N monoclonal antibody (V66)-Az-DBCO-PEG4-SPDMV-PMO.

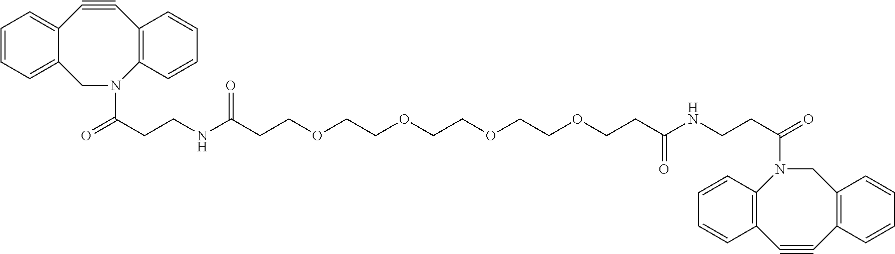 [SMILES: O=C(CCOCCOCCOCCOCCC(=O)NCCC(=O)N1Cc2ccccc2C#Cc2ccccc21)NCCC(=O)N1Cc2ccccc2C#Cc2ccccc21]

### DBCO-PEG4-SPDMV

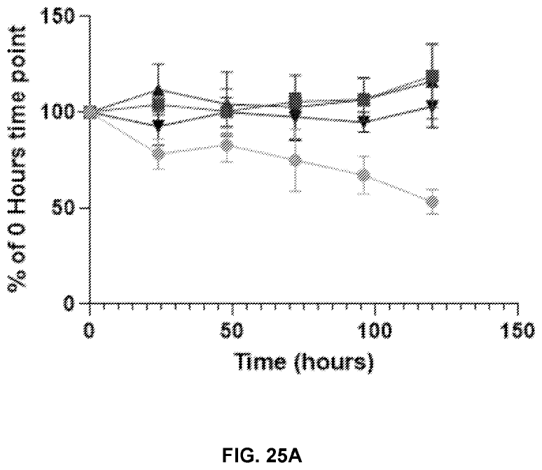

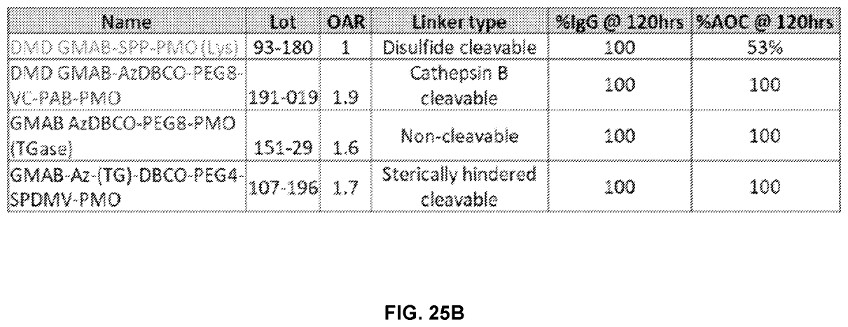

The results in FIG. 25A indicate that the di-methyl-hindered disulfide cleavable linker and protease cleavable linker have greater stability over a single methyl-hindered disulfide linker in mouse serum. FIG. 25B shows a data table indicating the panel of antibody-oligonucleotide conjugates (AOCs) tested and their oligonucleotide to antibody ratios (OAR).

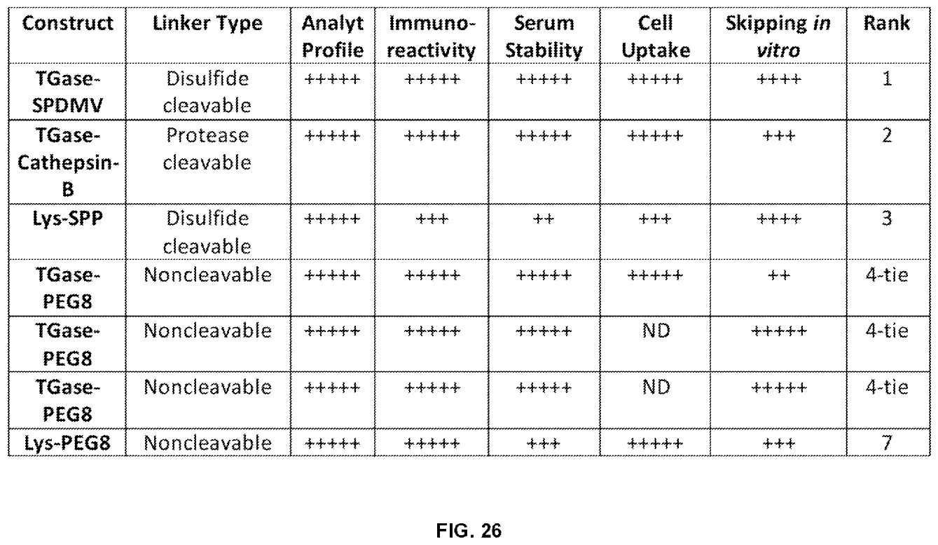

Furthermore, FIG. 26, shows the in vitro performance comparison of a panel of antibody-oligonucleotide constructs. The in vitro results suggest that transglutaminase-SPDMV constructs with a disulfide cleavable linker outperforms all other tested AOC constructs regarding analyte profile, immunoreactivity with nucleic acids, serum stability, cell uptake, and exon skipping in vitro.

Further assays will be tested to look at emergence of free payload (indicative of instability) and of OAR stability. Furthermore, plate based ELISAs and chromatographic methods (HIC) will be utilized to elucidate changes in OAR over time.

Sequences

| SEQ ID | | | |
|---|---|---|---|
| NO. | Name | Description | Sequence |
| 178 | NLS1 | Additionally | RASKSVSTSSYSYMHWYQQKPGQPPKLLIKY |
| | | contemplated | |
| | | sequences | |
| 179 | NLS2 | Additionally | RASKTVSTSSYSYMHWYQQKPGQPPKLLIKY |
| | | contemplated | |
| | | sequences | |
| 180 | NLS3 | Additionally | RVTITCRASKSVSTSSYSYMHWYQQKPGKAPKL |
| | | contemplated | |
| | | sequences | |
| 181 | NLS4 | Additionally | XRASKTVSTSSYSYMHWYQQKPGQPPKLLXKY (where (X) = any |
| | (consensus) | contemplated | residue, such as a basic residue (R or K) |
| | | sequences | |
| | Linker | Additionally | GS |
| | 0 | contemplated | |
| | | sequences | |
| 182 | Linker | Additionally | GQSSRSS |
| | 1 | contemplated | |
| | | sequences | |
| 183 | Linker | Additionally | GQSSRSSSGGGSSGGGGS |
| | 2 | contemplated | |
| | | sequences | |
| 184 | Linker | Additionally | GSGS |
| | 3 | contemplated | |
| | | sequences | |
| 185 | Linker | Additionally | GGGS |
| | 4 | contemplated | |
| | | sequences | |
| 186 | Linker | Additionally | (Gly₄-Ser)₂ |
| | 5 | contemplated | |
| | | sequences | |
| 187 | Linker | Additionally | (Gly₄-Ser)₄ |
| | 6 | contemplated | |
| | | sequences | |
| 188 | Linker | Additionally | (Gly-Gly-Gly-Gly-Ser)₃ |
| | 7 | contemplated | |
| | | sequences | |
| 189 | Linker | Additionally | RADAAPGGGGSGGGGSGGGGS |
| | 8 | contemplated | |
| | | sequences | |
| 190 | Linker | Additionally | ASTKGPSVFPLAPLESSGS |
| | 9 | contemplated | |
| | | sequences | |
| 191 | CDR | Additionally | GFTFSDYG |
| | H1.3 | contemplated | |
| | | sequences | |
| 192 | CDR | Additionally | GFTFSNYG |
| | H1.4 | contemplated | |
| | | sequences | |
| 193 | CDR | Additionally | ISSGSSTI |
| | H2.2 | contemplated | |
| | | sequences | |
| 194 | CDR | Additionally | ISSSSSTI |
| | H2.2 | contemplated | |
| | | sequences | |
| 195 | CDR | Additionally | ARRGLLLDY |
| | H3.2 | contemplated | |
| | | sequences | |
| 196 | CDR | Additionally | KSVSTSSYSY |
| | L1.2 | contemplated | |
| | | sequences | |
| 197 | CDR | Additionally | KTVSTSSYSY |
| | L1.2 | contemplated | |
| | | sequences | |
| | CDR | Additionally | YAS |
| | L2.2 | contemplated | |
| | | sequences | |
| 198 | CDR | Additionally | QHSREFPWT |
| | L3.2 | contemplated | |
| | | sequences | |
| 199 | | light chain | MTQSPSSLSASLGDRATITCRASKTVSTSSYSYMHWYQQKPGQPPKL |
| | | variable | LIKYASYLESGVPSRFSGSGSGTDFTLTISSLQPEDAATYYCQHSRE |
| | | domain (VL) | FPWTFGGGTKVEIK |
| 200 | | heavy chain | EVQLVESGGGLVQPGGSLRLSCAASGFTFSNYGMHWVRQAPGKGLEW |
| | | variable | VSYISSGSSTIYYADSVKGRFTISRDNAKNSLYLQMNSLRAEDTAVY |
| | | domain (VH) | YCARRGLLLDYWGQGTTVTVSS |

1. A conjugate of Formula (I): 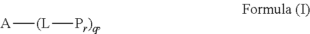
wherein in Formula (I):
A is an antibody or antigen-binding fragment thereof comprising a heavy chain variable region (VH) CDR1 comprising the amino acid sequence of SEQ ID NO:58, CDR2 comprising the amino acid sequence of SEQ ID NO:59, CDR3 comprising SEQ ID NO:60; and a light chain variable region (VL) CDR1 comprising the amino acid sequence of SEQ ID NO:61, CDR2 comprising the amino acid sequence of SEQ ID NO:62, CDR3 comprising the amino acid sequence of SEQ ID NO:63;
L is a linker;
P is an oligonucleotide moiety;
r is an integer from 1 to 4; and
q is an integer from 1 to 16.

2. The conjugate of claim 1, wherein the linker L comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}—, —N═CR^{a}—, —CR^{a}═N—, —S—, —S(O)—, —S(O)₂—, —OP(O)OR^{a}—, —OP(O)OR^{a}O—, —P(O)OR^{a}O—, —O—, —CR^{b}₂—, —[(CR^{b}₂)₁₋₁₂O]₁₋₅₀—, —C(O)—, —C(S)—, —C(═N—OH)—, —C(NR^{a})—, —C(NH₂Cl)—, —C(O)O—, —OC(O)—, —C(O)S—, —SC(O)—, —C(O)NR^{a}—, —NR^{a}C(O)—, —C(O)NR^{a}SO₂—, —SO₂NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, —S(O)ₜN(R^{a})ₜ— (where t is 1 or 2), —N(R^{a})S(O)ₜ— (where t is 1 or 2), and —X_{AA}—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted alkenyl, optionally substituted fluoroalkenyl, optionally substituted cycloalkenyl, optionally substituted cycloalkenylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
two independent R^{b} groups or an R^{a} and an R^{b} can be joined together to form an optionally substituted cycle; and
—X_{AA}— is an amino acid sequence comprising 1 to 6 amino acid moieties.

3. The conjugate of claim 2, wherein each amino acid moiety of —X_{AA}— is independently selected from alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr), valine (Val), citrulline (Cit), and homocitrulline (HoCit).

4. The conjugate of any one of claims 1 to 3, wherein the linker L comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, [CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}═N—, —S—, —OP(O)OR^{a} O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—.

5. The conjugate of any one of claims 1 to 4, wherein the linker L comprises one or more groups selected from optionally substituted C₁-C₁₆ alkylene, —C≡C—, —CR^{a}═CR^{a}—, optionally substituted phenylene, optionally substituted C₃-C₆ cycloalkylene, —[CH₂CH₂O]₁₋₁₆—, —[CH₂CH₂CH₂O]₁₋₁₆—, optionally substituted 5- to 6-membered heteroarylene, optionally substituted 5- to 20-membered heterocycloalkylene, —NR^{a}—, —N═CR^{a}—, —CR^{a}—N—, —S—, —OP(O)OR^{a} O—, —O—, —CR^{b}₂—, —C(O)—, —C(O)O—, —OC(O)—, —C(O)NR^{a}—, —NR^{a}C(O)—, —OC(O)O—, —OC(O)NR^{a}—, —NR^{a}C(O)O—, and —X_{AA}—.

6. The conjugate of any one of claims 1 to 5, wherein the linker L comprises one or more groups selected from —[C(R^{b})₂]₁₋₆—, —C≡C—, —CR^{a}═CR^{a}—, —[CH₂CH₂O]₁₋₁₆—, —NR^{a}—, —N═CR^{a}—, —CR^{a}═N—, —S—, —OP(O)OR^{a} O—, —O—, —C(O)—, —C(O)O—, —OC(O)—, —C(O)NR^{a}—, —NR^{a}C(O)—, —OC(O)O—, —OC(O)NR^{a}—, —NR^{a}C(O)O—, —X_{AA}—, 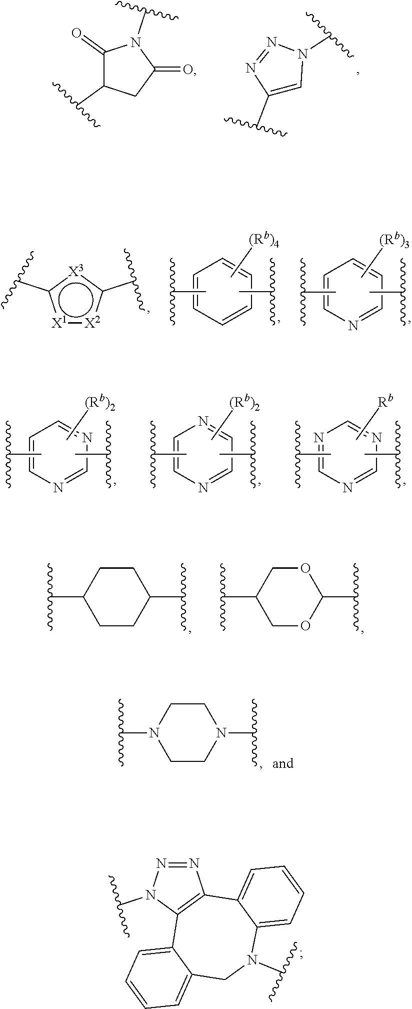
and
X¹, X², and X³ are independently selected at each occurrence from NR^{a}, N, CR^{b}, S, and O.

7. The conjugate of any one of claims 1 to 6, wherein the linker L is of Formula (L-1): 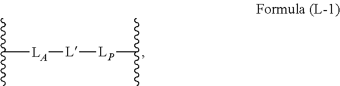
wherein in Formula (L-1):
L_{A} is a connecting moiety through which A is covalently attached to L′;
L′ is a bond or comprises one or more groups selected from optionally substituted alkylene, optionally substituted alkenylene, optionally substituted alkynylene, optionally substituted arylene, optionally substituted cycloalkylene, optionally substituted heteroalkylene, optionally substituted heteroarylene, optionally substituted heterocycloalkylene, —NR^{a}—, —N═CR^{a}—, —CR^{a}—N—, —S—, —S(O)—, —S(O)₂—, —OP(O)OR^{a} O—, —O—, —CR^{b}₂—, —[(CR^{b}₂)₁₋₁₂O]₁₋₅₀—, —C(O)—, —C(S)—, —C(NR^{a})—, —C(NH₂Cl)—, —C(O)O—, —OC(O)—, —C(O)S—, —SC(O)—, —C(O)NR^{a}—, —NR^{a}C(O)—, —C(O)NR^{a} SO₂—, —SO₂NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, —S(O)ₜN(R^{a})—, —N(R^{a})S(O)ₜ—, and —X_{AA}—; and
L_{P} is a connecting moiety through which P is covalently attached to L′.

8. The conjugate of any one of claims 1 to 7, wherein the linker L comprises at least one cleavable moiety.

9. The conjugate of claim 8, wherein the cleavable moiety comprises an acid-labile moiety, a reducibly-labile moiety, or an enzymatically-labile moiety.

10. The conjugate of claim 8 or claim 9, wherein the cleavable moiety comprises one or more groups selected from: 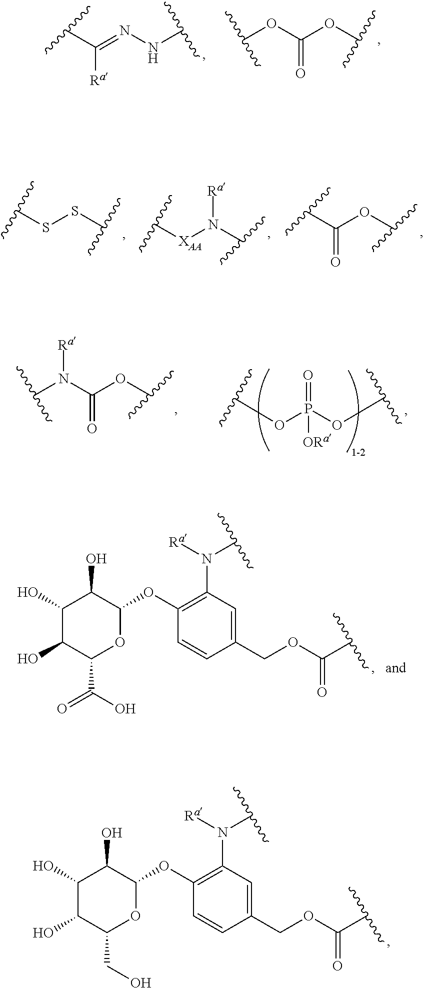
wherein:
each R^{a′} is independently selected at each occurrence from hydrogen, optionally substituted alkyl, and optionally substituted heteroalkyl.

11. The conjugate of any one of claims 9 to 10, wherein the cleavable moiety comprises the reducibly-labile moiety —S—S—.

12. The conjugate of any one of claims 1 to 8, wherein the linker L is of Formula (L-10): 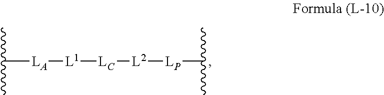
wherein in Formula (L-10):
L_{A} is selected from a bond, —NR^{a′}—, and —S—;
L¹ is a bond or comprises one or more groups selected from optionally substituted C₁-C₁₈ alkylene, —C≡C—, —CR^{a}═CR^{a}—, optionally substituted 6- to 14-membered arylene, optionally substituted d C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}—N—, —S—, —OP(O)OR^{a} O—, —O—, —CR^{b}₂—, —C(O)—, —C(O)O—, —OC(O)—, —C(O)S—, —SC(O)—, —C(O)NR^{a}—, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—;
L_{C} is selected from an acid-labile moiety, a reducibly-labile moiety, and an enzymatically-labile moiety;
L² is a bond or comprises one or more groups selected from optionally substituted C₁-C₁₈ alkylene, —C≡C—, —CR^{a}═CR^{a}, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —N═CR^{a}—, —CR^{a}—N—, —S—, —OP(O)OR^{a} O—, —O—, —CR^{b}₂—, —C(O)—, —C(O)O—, —OC(O)—, —C(O)S—, —SC(O)—, —C(O)NR^{a}, —NR^{a}C(O)—, —OC(O)O—, —OC(O)S—, —SC(O)O—, —OC(O)NR^{a}—, —NR^{a}C(O)O—, —SC(O)NR^{a}—, —NR^{a}C(O)S—, and —X_{AA}—;
L_{P} is selected from a bond, —NR^{a′}—, —S—, and —O—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{a′} is independently selected at each occurrence from hydrogen, optionally substituted alkyl, and optionally substituted heteroalkyl;
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl; and
—X_{AA}— is an amino acid sequence comprising 1 to 4 amino acid moieties.

13. The conjugate of claim 11, wherein L_{C} is selected from: 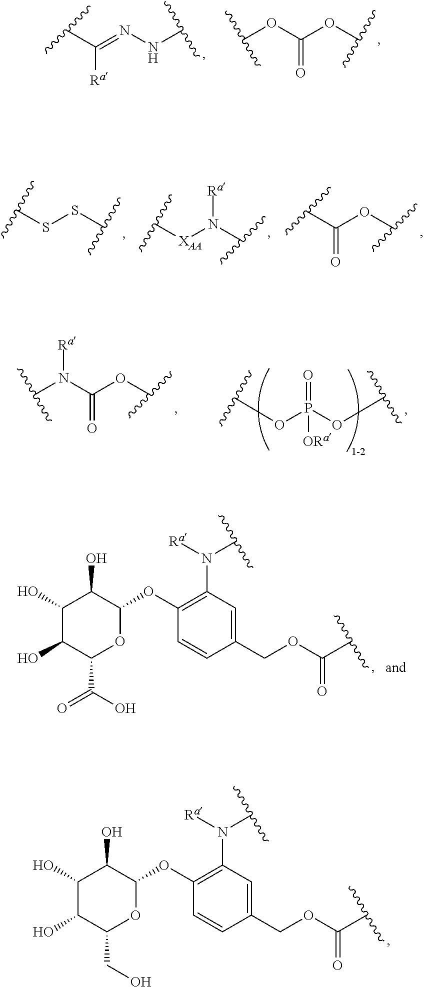
wherein:
each R^{a′} is independently selected at each occurrence from hydrogen, optionally substituted alkyl, and optionally substituted heteroalkyl.

14. The conjugate of claim 12 or claim 13, wherein L_{C} is —S—S—.

15. The conjugate of any one of claims 1 to 7, wherein the linker L is of Formula (L-11): 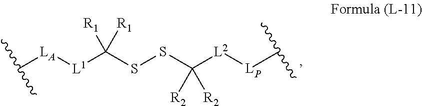
wherein in Formula (L-11):
L_{A} is selected from a bond, —NH—, and —S—;
L¹ is a bond or comprises one or more groups selected from —[C(R^{b})₂]₁₋₆—, —C≡C—, —CR^{a}═CR^{a}—, —[CH₂CH₂O]₁₋₆—, —NR^{a}—, —N═CR^{a}—, —CR^{a}═N—, —S—, —OP(O)OR^{a} O—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—, 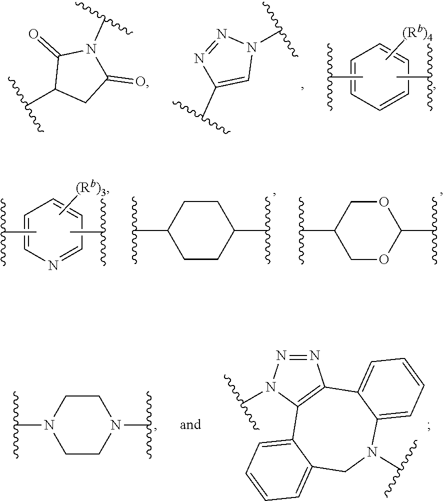
L² is a bond or comprises one or more groups selected from —[C(R^{b})₂]₁₋₆—, —C≡C—, —CR^{a}═CR^{a}—, —[CH₂CH₂O]₁₋₆—, —NR^{a}—, —N═CR^{a}—, —CR^{a}═N—, —S—, —OP(O)OR^{a} O—, —O—, —C(O)—, —C(O)O—, —OC(O)—, —C(O)NR^{a}—, —NR^{a}C(O)—, —OC(O)O—, —X_{AA}—, —OC(O)NR^{a}—, —NRC(O)O—, 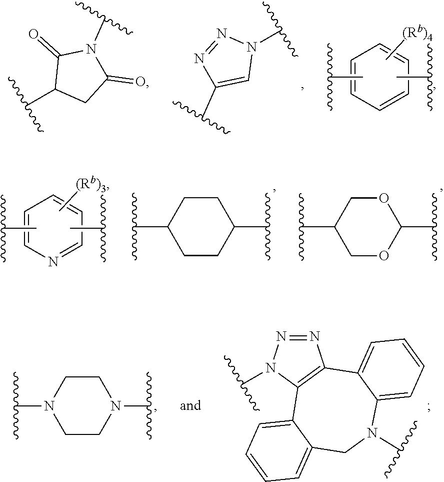
L_{P} is selected from a bond, —NR^{a′}, and —O—;
each R₁ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₁ groups are taken together to form optionally substituted cycloalkyl;
each R₂ is independently selected from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or both R₂ groups are taken together to form optionally substituted cycloalkyl;
each R^{a} is independently selected at each occurrence from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted alkyl;
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl; and
—X_{AA}— is an amino acid sequence comprising 2 to 4 amino acid moieties.

16. The conjugate of any one of claims 1 to 7, wherein the linker L is of Formula (L-12): 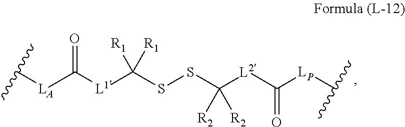
wherein in Formula (L-12):
L_{A} is selected from a bond and —NH—;
L^{1′} comprises one or more groups selected from —[C(R^{b})₂]₁₋₁₀—, —[CH₂CH₂O]₁₋₁₀—, —NR^{a}—, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—, 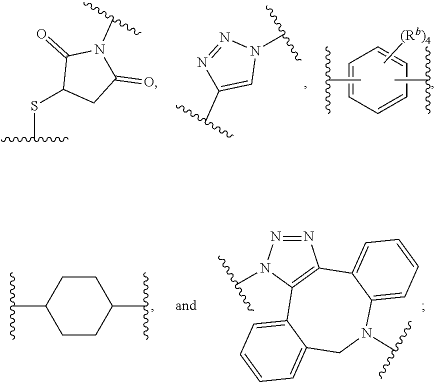
L^{2′} comprises one or more groups selected from —[C(R^{b})₂]₁₋₁₀—, —[CH₂CH₂O]₁₋₁₀—, —NR^{a}, —O—, —C(O)—, —C(O)O—, —OC(O)—, —C(O)NR^{a}—, —NR^{a}C(O)—, —OC(O)O—, —X_{AA}—, —OC(O)NR^{a}—, —NR^{a}C(O)O—, 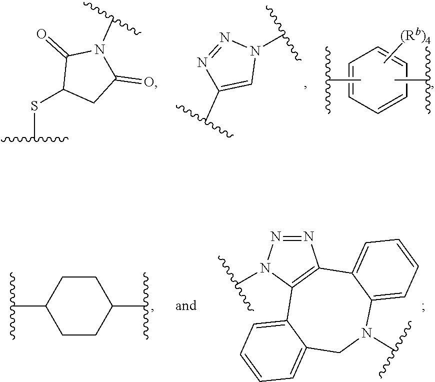
L_{P} is selected from a bond and —NR^{a′}—;
each R₁ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₂ groups are taken together to form optionally substituted C₃-C₆ cycloalkyl;
each R₂ is independently selected from hydrogen, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or both R₂ groups are taken together to form optionally substituted C₃-C₆ cycloalkyl;
each R^{a} is independently selected at each occurrence from hydrogen, optionally substituted C₁-C₆ alkyl, optionally substituted C₁-C₆ fluoroalkyl, and optionally substituted C₃-C₆ cycloalkyl;
each R^{a′} is independently selected at each occurrence from hydrogen and optionally substituted C₁-C₆ alkyl;
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₆ alkyl, optionally substituted C₁-C₆ fluoroalkyl, and optionally substituted C₃-C₆ cycloalkyl; or two independent R^{b} groups attached to the same carbon atom are taken together to form optionally substituted C₃-C₆ cycloalkyl; and
—X_{AA}— is an amino acid sequence comprising 2 or 3 amino acid moieties.

17. The conjugate of claim 15 or claim 16, wherein at least one R₁ or R₂ is other than hydrogen.

18. The conjugate of any one of claims 15 to 17, wherein at least one R₁ is an optionally substituted C₁-C₈ alkyl.

19. The conjugate of any one of claims 15 to 17, wherein each R₁ is independently an optionally substituted C₁-C₈ alkyl.

20. The conjugate of any one of claims 15 to 19, wherein at least one R₂ is an optionally substituted C₁-C₈ alkyl.

21. The conjugate of any one of claims 15 to 19, wherein each R₂ is independently an optionally substituted C₁-C₈ alkyl.

22. The conjugate of any one of claims 1 to 10, wherein the linker L is selected from:

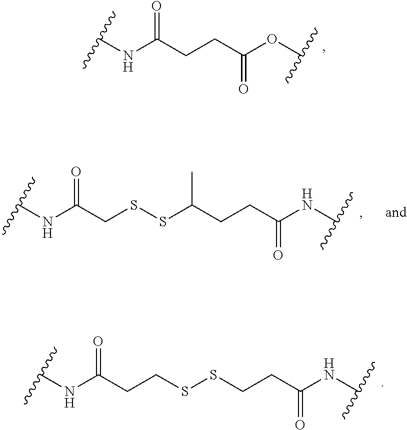 [SMILES: *NC(=O)CCC(=O)O* ; *NC(=O)CCC(C)SSCC(=O)N* ; *NC(=O)CCSSCCC(=O)N*]

23. The conjugate of any one of claims 1 to 16, wherein the linker L is selected from:

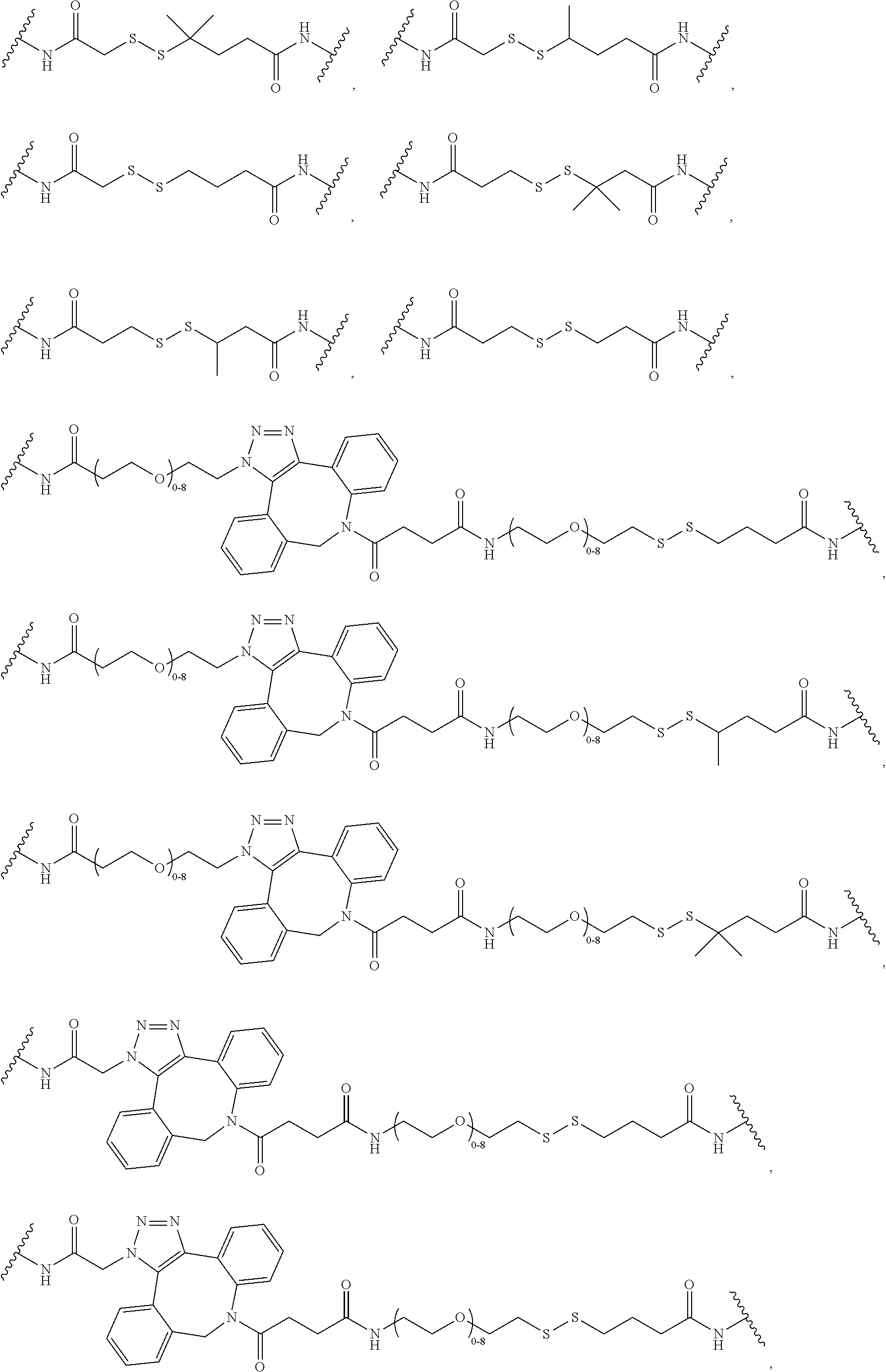

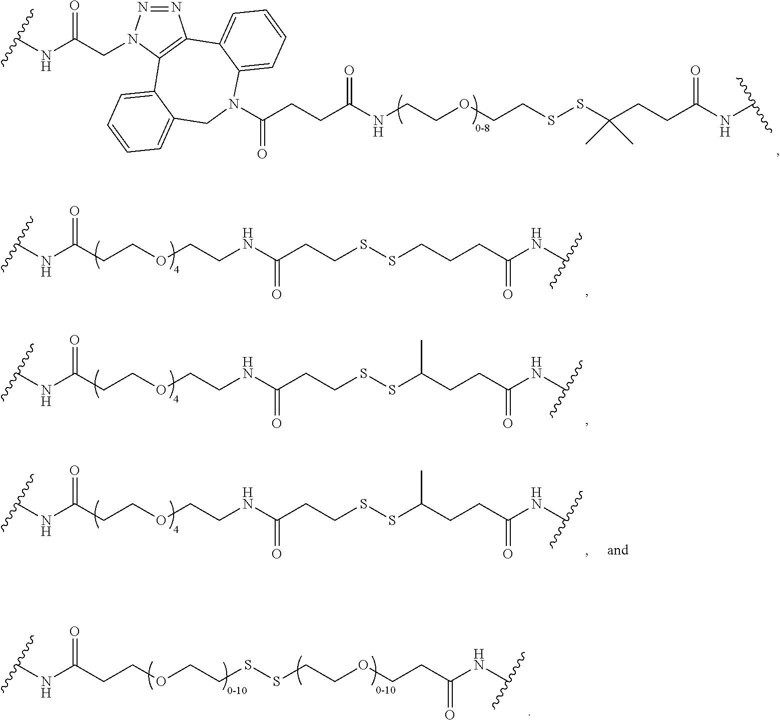

24. The conjugate of any one of claims 8 to 23, wherein the linker is a cleavable linker.

25. The conjugate of claim 24, wherein the cleavable linker is conjugated to a lysine of the 3E10 antibody or antigen-binding fragment thereof.

26. The conjugate of claim 24, wherein the cleavable linker is conjugated to a cysteine of the 3E10 antibody or antigen-binding fragment thereof.

27. The conjugate of claim 24, wherein the cleavable linker is conjugated to a histidine of the 3E10 antibody or antigen-binding fragment thereof.

28. The conjugate of claim 24, wherein the cleavable linker is conjugated to an arginine of the 3E10 antibody or antigen-binding fragment thereof.

29. The conjugate of claim 24, wherein the cleavable linker is conjugated to an aspartic acid of the 3E10 antibody or antigen-binding fragment thereof.

30. The conjugate of claim 24, wherein the cleavable linker is conjugated to a glutamine of the 3E10 antibody or antigen-binding fragment thereof.

31. The conjugate of claim 30, wherein the conjugate has a drug to antibody ratio (DAR) of at least 3:1.

32. The conjugate of claim 30, wherein the conjugate has a drug to antibody ratio (DAR) of at least 4:1.

33. The conjugate of claim 30, wherein the conjugate has a drug to antibody ratio (DAR) of at least 6:1.

34. The conjugate of any one of claims 30-33, wherein the linker is a branched linker attached to at least two copies of the oligonucleotide moiety.

35. The conjugate of claim 24, wherein the cleavable linker is conjugated to a serine of the 3E10 antibody or antigen-binding fragment thereof.

36. The conjugate of claim 24, wherein the cleavable linker is conjugated to a threonine of the 3E10 antibody or antigen-binding fragment thereof.

37. The conjugate of claim 24, wherein the cleavable linker is conjugated to a tyrosine of the 3E10 antibody or antigen-binding fragment thereof.

38. The conjugate of any one of claims 1 to 7, wherein the linker L is of Formula (L-20): 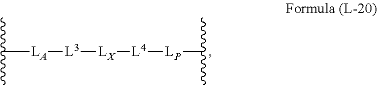
wherein in Formula (L-20):
L_{A} is selected from a bond, —NR^{a′}—, and —S—;
L³ is a bond or comprises one or more groups selected from —[C(R^{b})₂]₁₋₈—, —NR^{a}—, —C(O)—, —C(S)—, —C(NR^{a})—, —C(NH₂Cl)—, —C≡C—, —CR^{a}—CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
L_{X} comprises one or more groups selected from optionally substituted C₁-C₁₈ alkylene, —C≡C—, —CR^{a}═CR^{a}—, optionally substituted 6- to 14-membered arylene, optionally substituted C₃-C₂₀ cycloalkylene, —[CH₂O]₁₋₁₈—, —[CH₂CH₂O]₁₋₁₈—, —[CH₂CH₂CH₂O]₁₋₁₈—, optionally substituted 5- to 18-membered heteroarylene, optionally substituted 3- to 20-membered heterocycloalkylene, —NR^{a}—, —S—, —O—, —CR^{b}₂—, —C(O)—, —C(S)—, —C(NR^{a})—, —C(NH₂Cl)—, —C(O)O—, —OC(O)—, —C(O)S—, —SC(O)—, —C(O)NR^{a}—, and —NR^{a}C(O)—;
L⁴ is a bond or comprises one or more groups selected from —[C(R^{b})₂]₁₋₈—, —NR^{a}—, —C(O)—, —C(S)—, —C(NR^{a})—, —C(NH₂Cl)—, —C≡C—, —CR^{a}═CR^{a}—, optionally substituted 6- to 14-membered arylene, optionally substituted C₃-C₂₀ cycloalkylene, optionally substituted 5- to 18-membered heteroarylene, and optionally substituted 3- to 20-membered heterocycloalkylene;
L_{P} is selected from a bond, —NR^{a′}—, —S—, and —O—;
each R^{a} is independently selected at each occurrence from hydrogen, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl;
each R^{a′} is independently selected at each occurrence from hydrogen, optionally substituted alkyl, and optionally substituted heteroalkyl; and
each R^{b} is independently selected at each occurrence from hydrogen, halide, —OH, —SO₃H, —OPO₃H₂, —PO₃H₂, —C(O)NR^{a}₂, —CO₂R^{a}, —NR^{a}₂, optionally substituted alkyl, optionally substituted fluoroalkyl, optionally substituted cycloalkyl, optionally substituted cycloalkylalkyl, optionally substituted aryl, optionally substituted aralkyl, optionally substituted heteroalkyl, optionally substituted heterocycloalkyl, optionally substituted heterocycloalkylalkyl, optionally substituted heteroaryl, and optionally substituted heteroarylalkyl; or two independent R^{b} groups are taken together to form optionally substituted cycloalkyl, optionally substituted aryl, optionally substituted heterocycloalkyl, or optionally substituted heteroaryl.

39. The conjugate of any one of claims 1 to 7, wherein the linker L is of Formula (L-21): 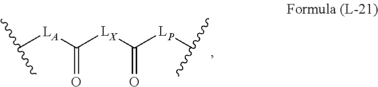
wherein in Formula (L-21):
L_{A} is selected from a bond and —NH—;
L_{X} comprises one or more groups selected from optionally substituted —[C(R^{b})₂]₁₋₆—, —C≡C—, —CR^{a}—CR^{a}—, —[CH₂CH₂CH₂O]₁₋₆—, —NR^{a}—, —O—, —C(O)—, —C(O)O—, —OC(O)—, —C(O)S—, —SC(O)—, —C(O)NR^{a}—, —NR^{a}C(O)—, 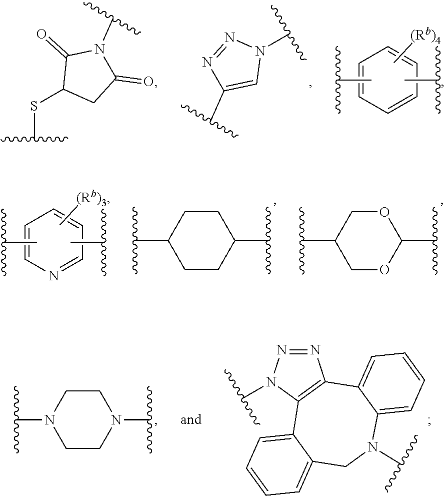
L_{P} is selected from a bond and —NR^{a′}—;
each R^{a} is independently selected at each occurrence from hydrogen, optionally substituted C₁-C₈alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

40. The conjugate of any one of claims 1 to 7, wherein the linker L is of Formula (L-22a) or Formula (L-22b): 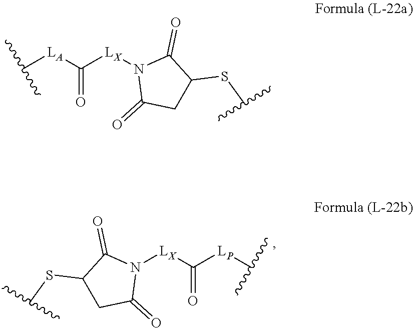
wherein in Formulas (L-22a) and (L-22b):
L_{A} is selected from a bond and —NH—;
L_{X} comprises one or more groups selected from optionally substituted —[C(R^{b})₂]₁₋₁₀—, —C≡C—, —CR^{a}—CR^{a}—, —[CH₂CH₂CH₂O]₁₋₁₀—, —NR^{a}—, —C(O)—, —C(O)O—, —OC(O)—, —C(O)S—, —SC(O)—, —C(O)NR^{a}—, —NR^{a}C(O)—, 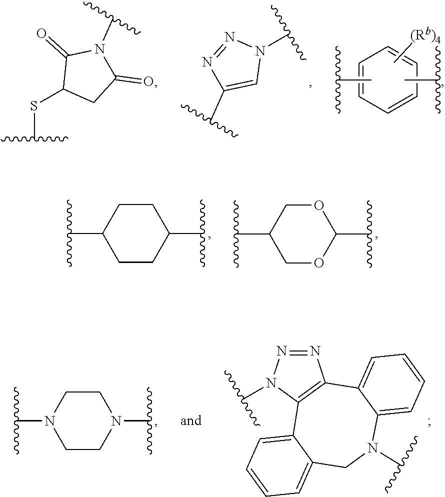
L_{P} is selected from a bond and —NR^{a′}—;
each R^{a} is independently selected at each occurrence from hydrogen, optionally substituted C₁-C₈alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl;
each R^{a′} is independently selected at each occurrence from hydrogen and optionally substituted C₁-C₆ alkyl; and
each R^{b} is independently selected at each occurrence from hydrogen, halide, —OH, —SO₃H, —OPO₃H₂, —PO₃H₂, —CO₂R^{a}, —NR^{a}₂, optionally substituted C₁-C₈ alkyl, optionally substituted C₁-C₈ fluoroalkyl, optionally substituted C₃-C₆ cycloalkyl, optionally substituted phenyl, optionally substituted benzyl, optionally substituted 5- to 10-membered heterocycloalkyl, optionally substituted 5- to 6-membered heteroaryl; or two independent R^{b} groups are taken together to form optionally substituted cycloalkyl.

41. The conjugate of any one of claims 1 to 7, wherein the linker L is selected from:

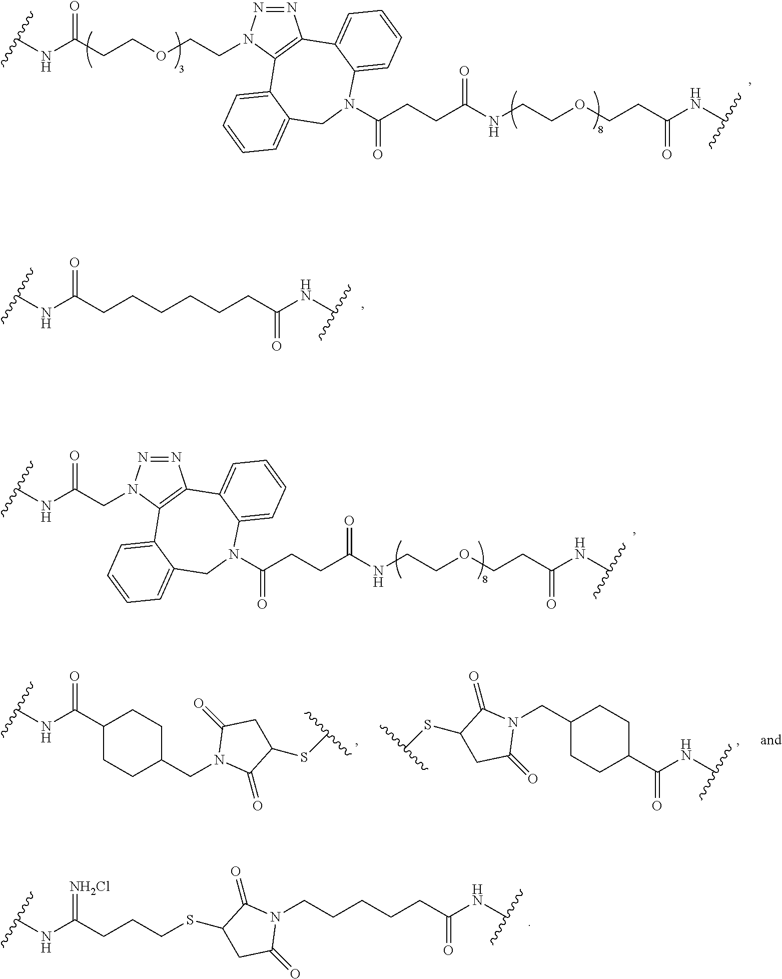

42. The conjugate of any one of claims 38 to 41, wherein the linker is a non-cleavable linker.

43. The conjugate of claim 42, wherein the non-cleavable linker is conjugated to a lysine of the 3E10 antibody or antigen-binding fragment thereof.

44. The conjugate of claim 42, wherein the non-cleavable linker is conjugated to a cysteine of the 3E10 antibody or antigen-binding fragment thereof.

45. The conjugate of claim 42, wherein the non-cleavable linker is conjugated to a histidine of the 3E10 antibody or antigen-binding fragment thereof.

46. The conjugate of claim 42, wherein the non-cleavable linker is conjugated to an arginine of the 3E10 antibody or antigen-binding fragment thereof.

47. The conjugate of claim 42, wherein the non-cleavable linker is conjugated to an aspartic acid of the 3E10 antibody or antigen-binding fragment thereof.

48. The conjugate of claim 42, wherein the non-cleavable linker is conjugated to a glutamine of the 3E10 antibody or antigen-binding fragment thereof.

49. The conjugate of claim 48, wherein the conjugate has a drug to antibody ratio (DAR) of at least 3:1.

50. The conjugate of claim 48, wherein the conjugate has a drug to antibody ratio (DAR) of at least 4:1.

51. The conjugate of claim 48, wherein the conjugate has a drug to antibody ratio (DAR) of at least 6:1.

52. The conjugate of any one of claims 48-51, wherein the linker is a branched linker attached to at least two copies of the oligonucleotide moiety.

53. The conjugate of claim 42, wherein the non-cleavable linker is conjugated to a serine of the 3E10 antibody or antigen-binding fragment thereof.

54. The conjugate of claim 42, wherein the non-cleavable linker is conjugated to a threonine of the 3E10 antibody or antigen-binding fragment thereof.

55. The conjugate of claim 42, wherein the non-cleavable linker is conjugated to a tyrosine of the 3E10 antibody or antigen-binding fragment thereof.

56. The conjugate of any one of claims 1 to 6, wherein the linker L comprises a moiety of Formula (L-100) or Formula (L-101): 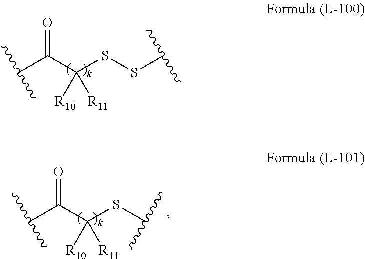
wherein in Formulas (L-100) and (L-101):
each R₁₀ is independently selected at each occurrence from hydrogen, —OH, and C₁-C₆ alkyl;
each R₁₁ is independently selected at each occurrence from hydrogen, —OH, and C₁-C₆ alkyl; and
k is 1, 2, 3, 4, 5, 6, 7, 8, 9 or 10.

57. The conjugate of claim 56, wherein each R₁₀ is hydrogen; each R₁₁ is independently selected at each occurrence from hydrogen, —CH₃, —CH₂CH₃, and —CH(CH₃)₂; and k is 3, 4, 5, 6, 7, 8, 9, or 10.

58. The conjugate of claim 56, wherein each R₁₀ is hydrogen; each R₁₁ is independently selected at each occurrence from hydrogen, —CH₃, and —CH₂CH₃; and k is 3, 4, 5, 6, 7, 8, 9, or 10.

59. The conjugate of claim 56, wherein each R₁₀ is hydrogen; each R₁₁ is independently selected at each occurrence from hydrogen and —CH₃; and k is 3, 4, 5, 6, 7, 8, 9, or 10.

60. The conjugate of claim 56, wherein each R₁₀ is hydrogen; each R¹¹ is hydrogen; and k is 3, 4, 5, 6, 7, 8, 9, or 10.

61. The conjugate of claim 1, wherein the linker L is selected from:

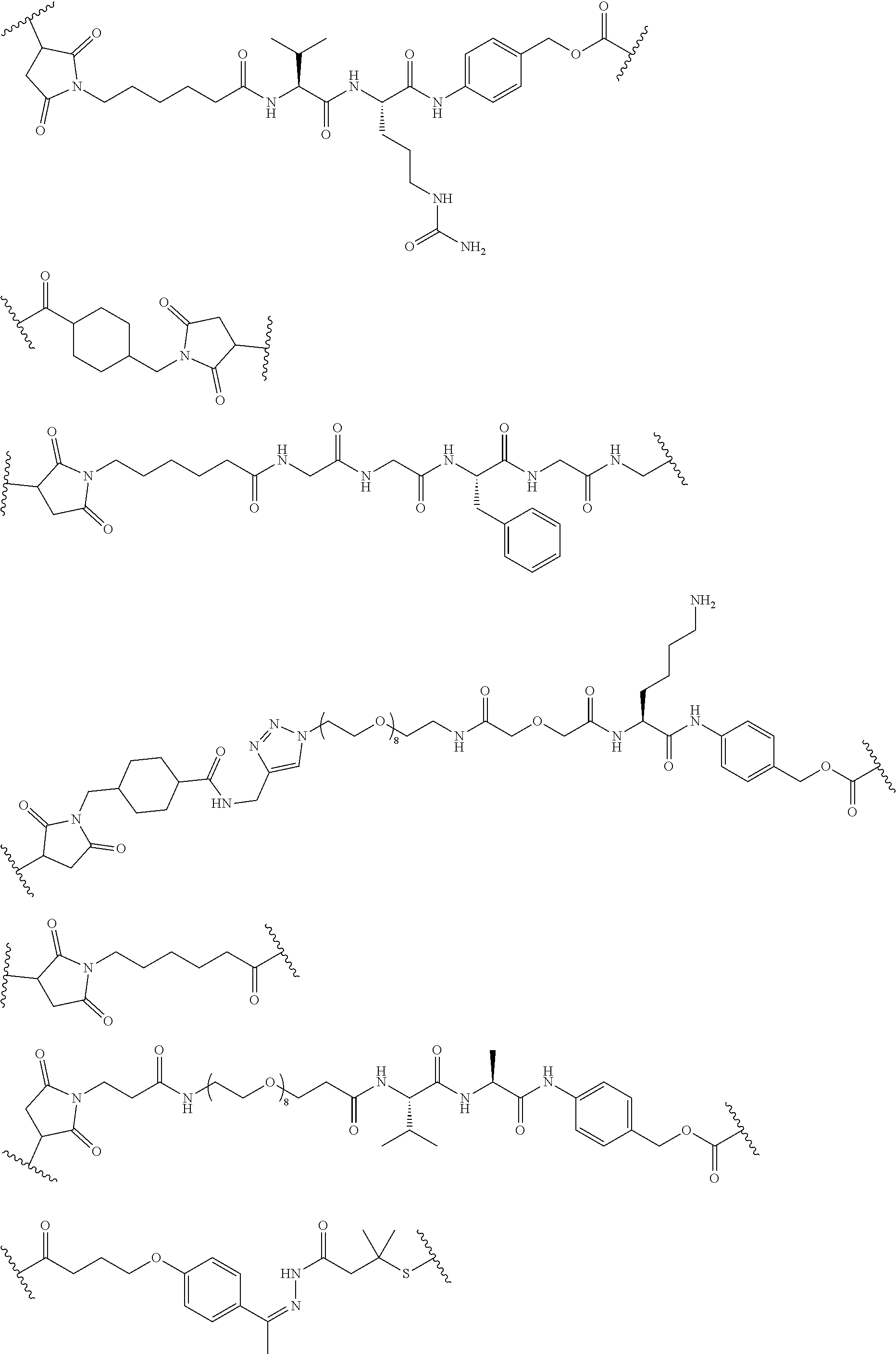

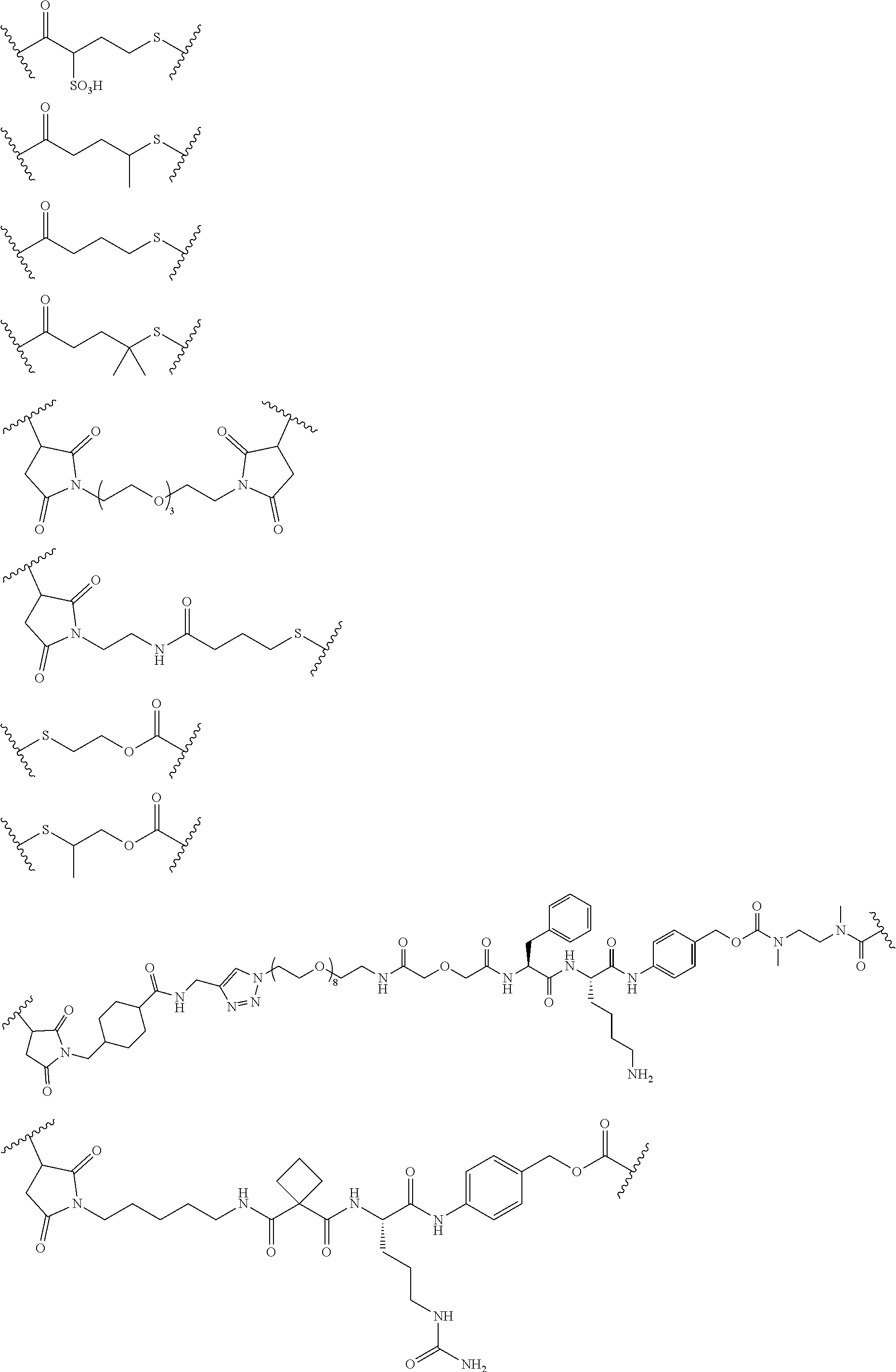

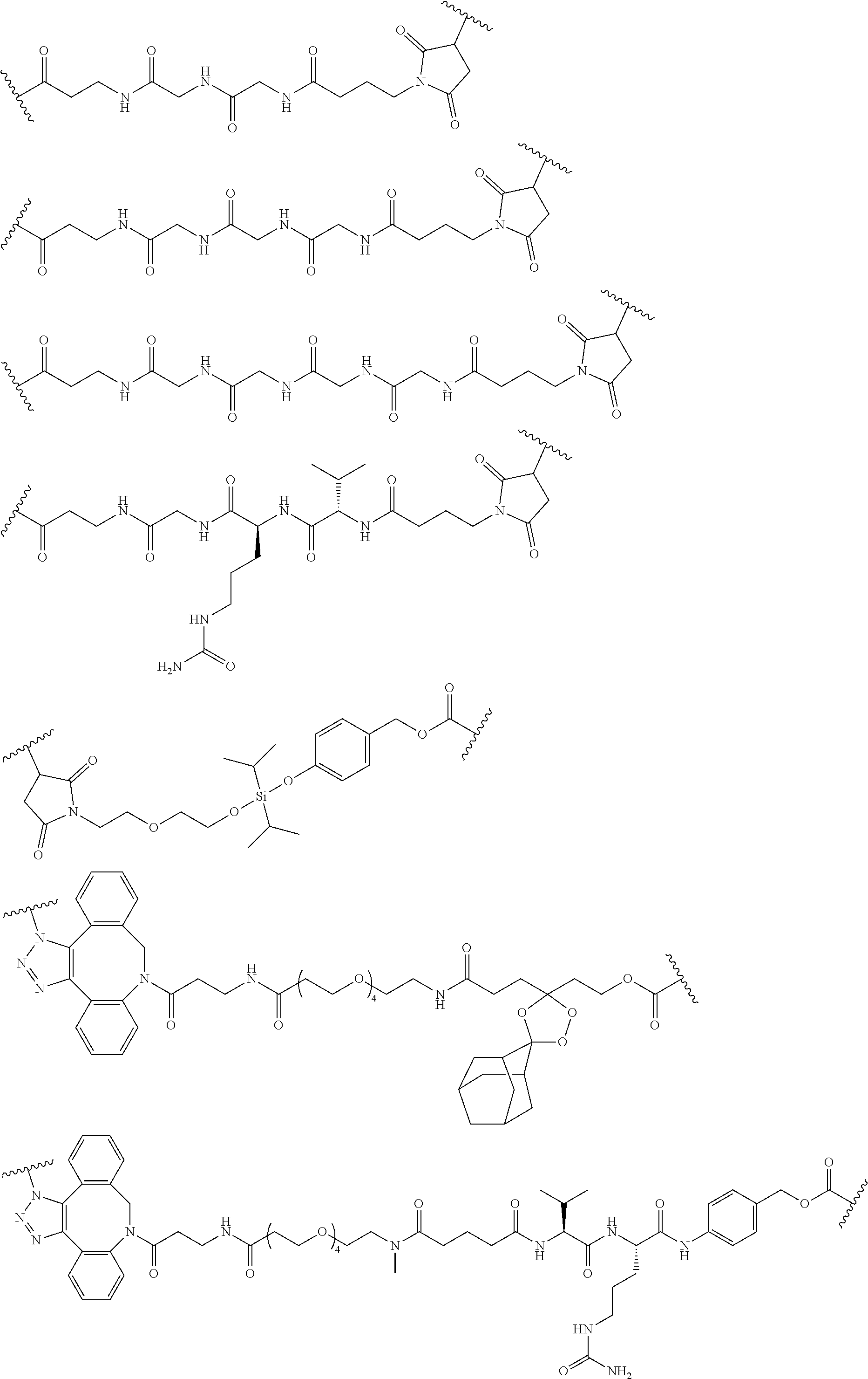

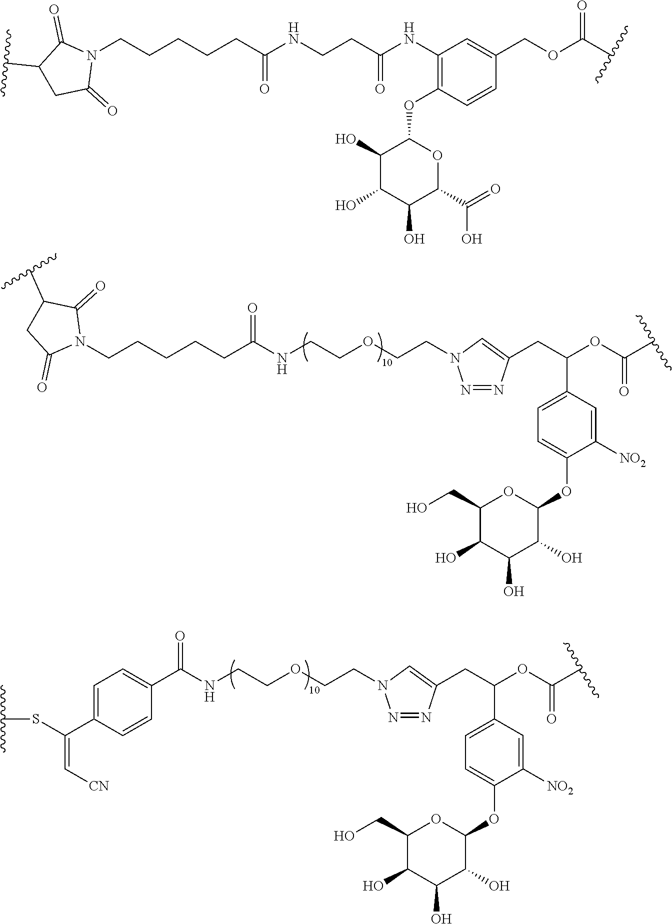

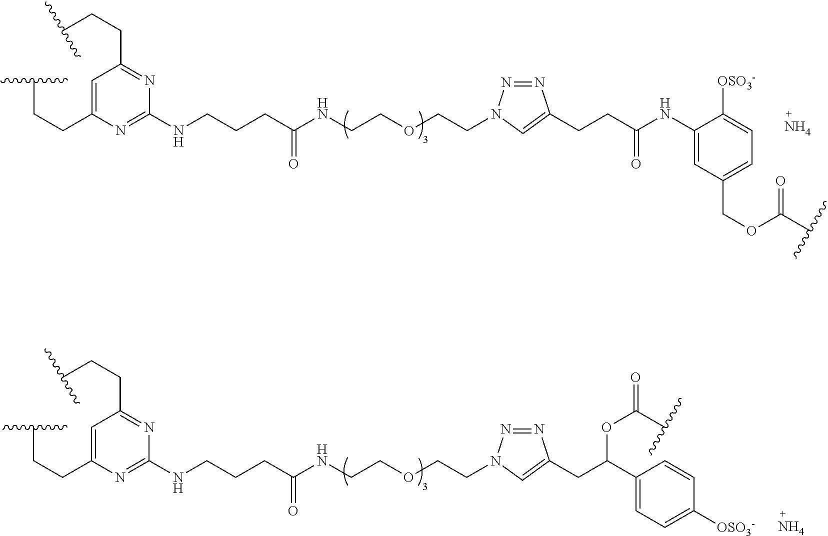

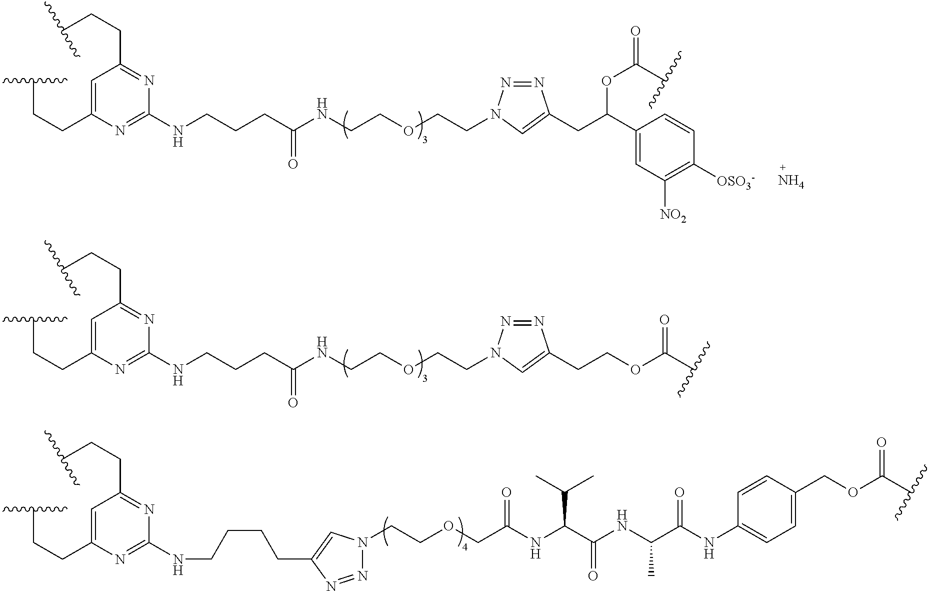

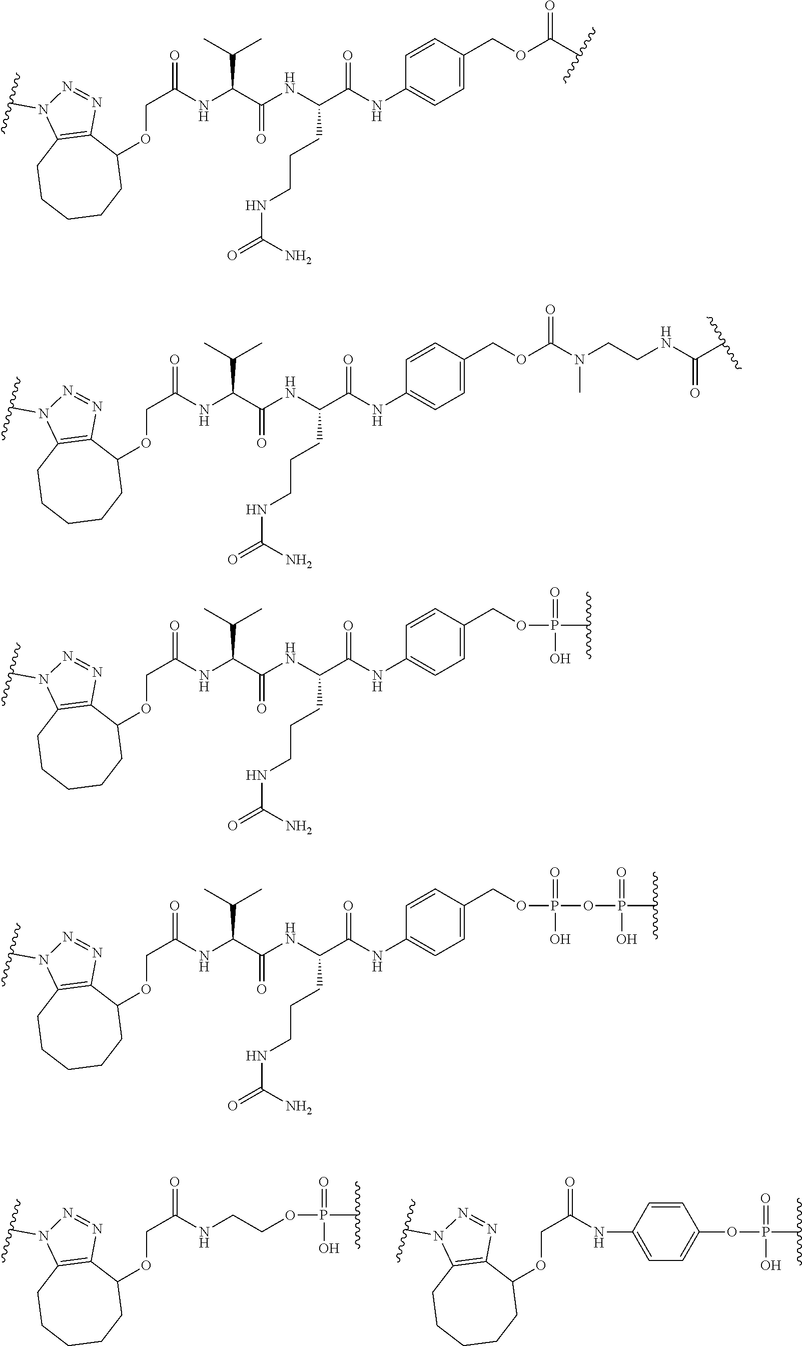 [SMILES: *C(=O)OCc1ccc(NC(=O)[C@H](CCCNC(N)=O)NC(=O)[C@@H](NC(=O)COC2CCCCCc3c2nnn3*)C(C)C)cc1 ; *C(=O)NCCN(C)C(=O)OCc1ccc(NC(=O)[C@H](CCCNC(N)=O)NC(=O)[C@@H](NC(=O)COC2CCCCCc3c2nnn3*)C(C)C)cc1 ; *n1nnc2c1CCCCCC2OCC(=O)N[C@H](C(=O)N[C@@H](CCCNC(N)=O)C(=O)Nc1ccc(COP(*)(=O)O)cc1)C(C)C ; *n1nnc2c1CCCCCC2OCC(=O)N[C@H](C(=O)N[C@@H](CCCNC(N)=O)C(=O)Nc1ccc(COP(=O)(O)OP(*)(=O)O)cc1)C(C)C ; *n1nnc2c1CCCCCC2OCC(=O)NCCOP(*)(=O)O ; *n1nnc2c1CCCCCC2OCC(=O)Nc1ccc(OP(*)(=O)O)cc1]

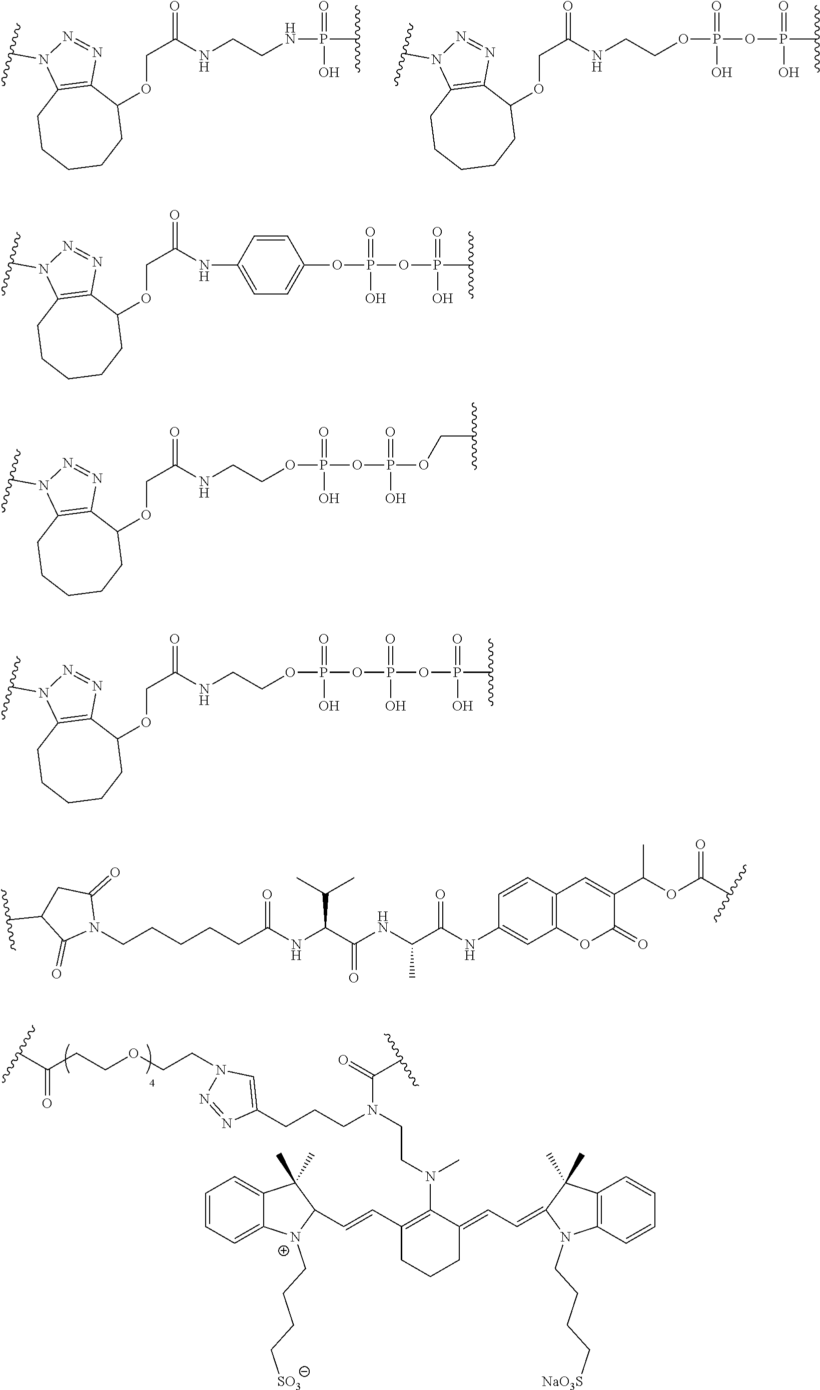

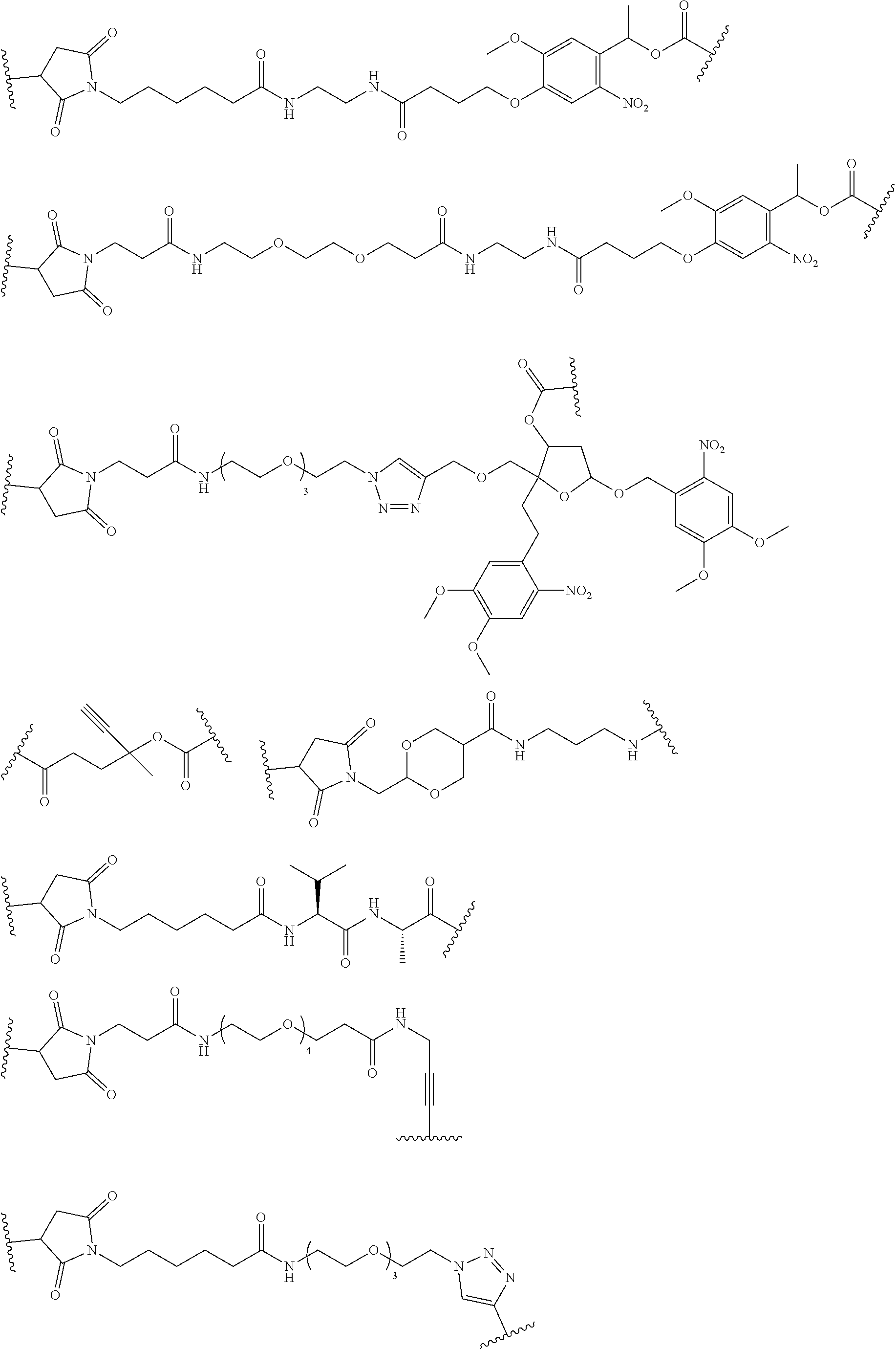

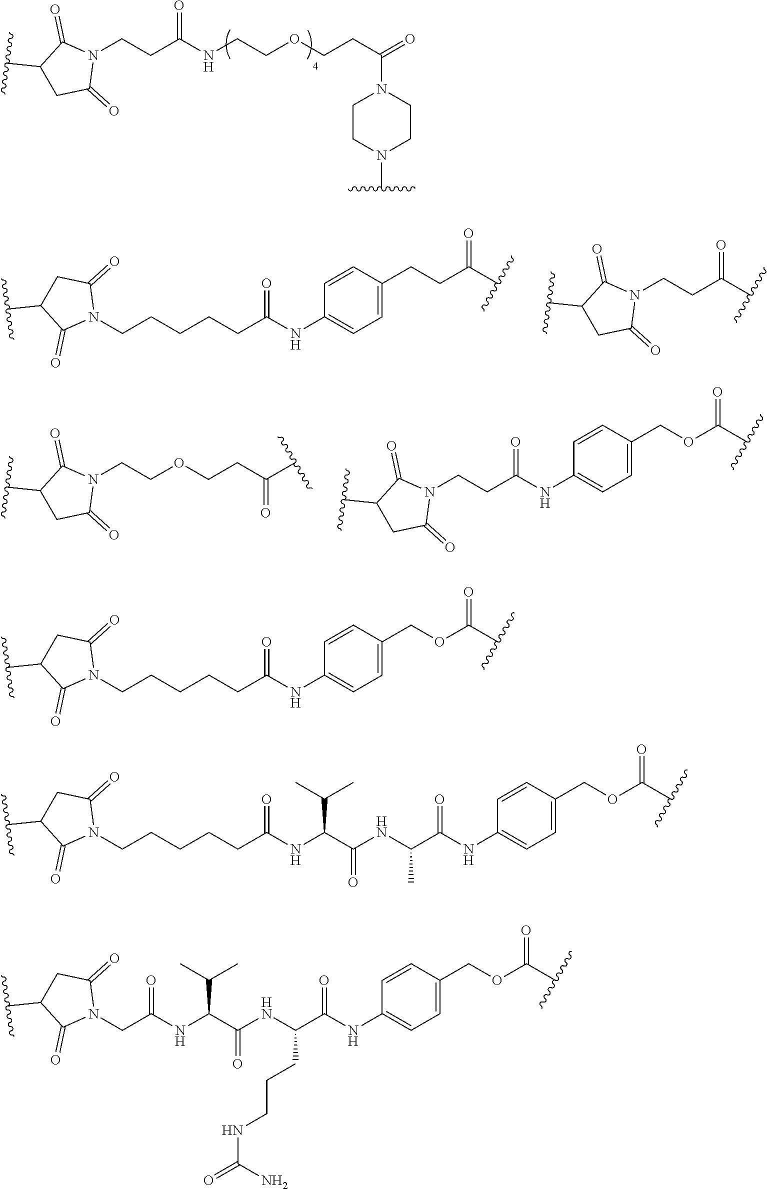

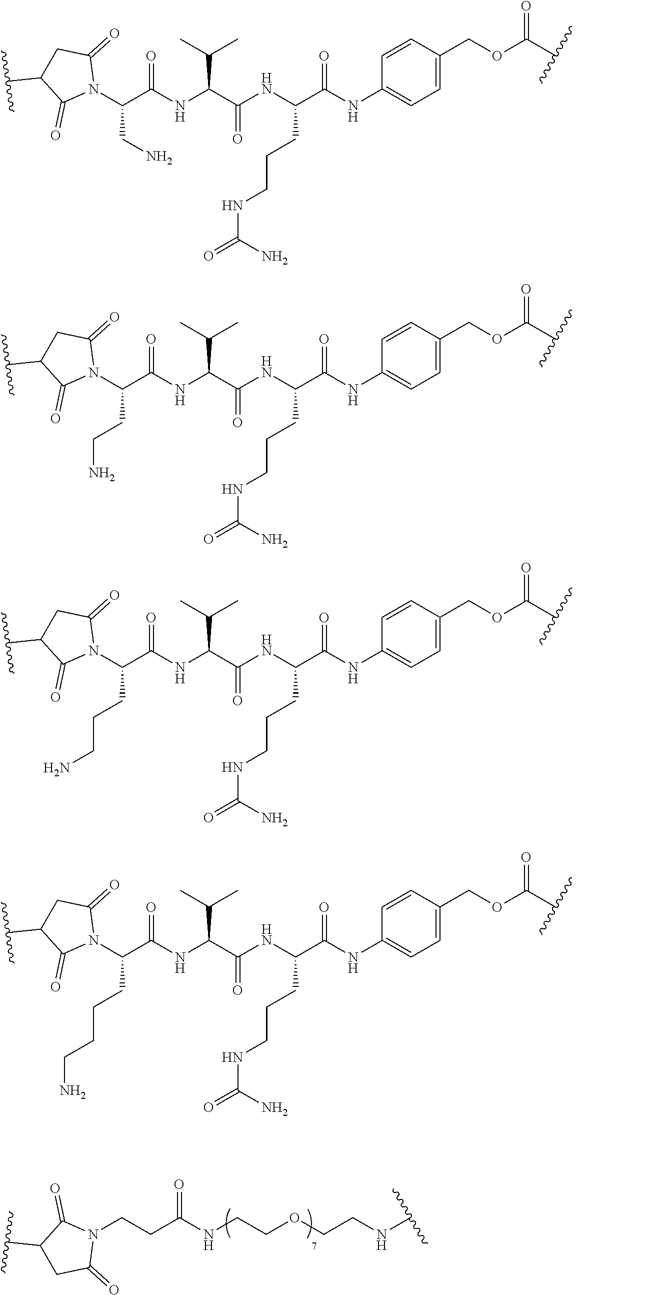

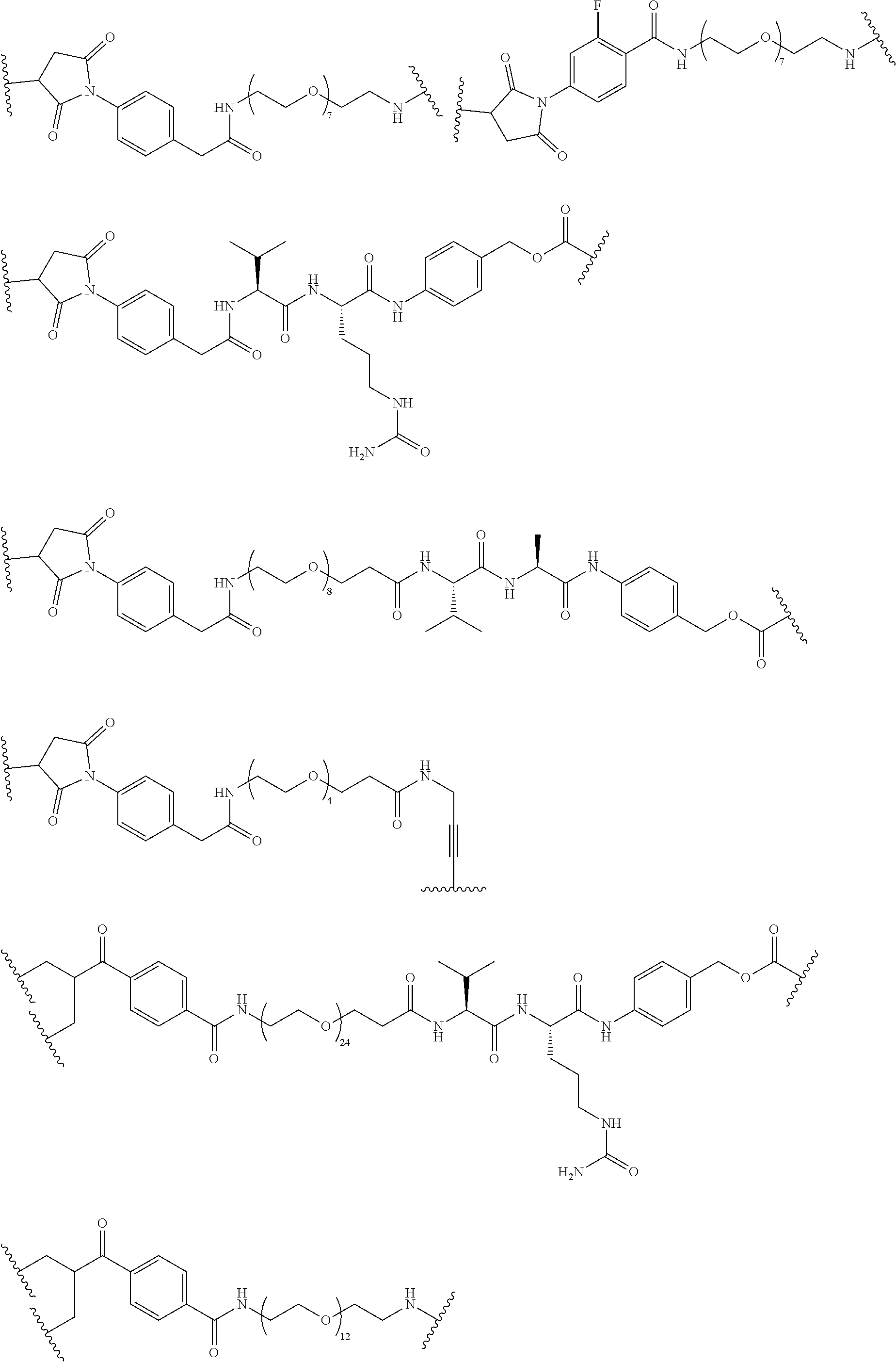

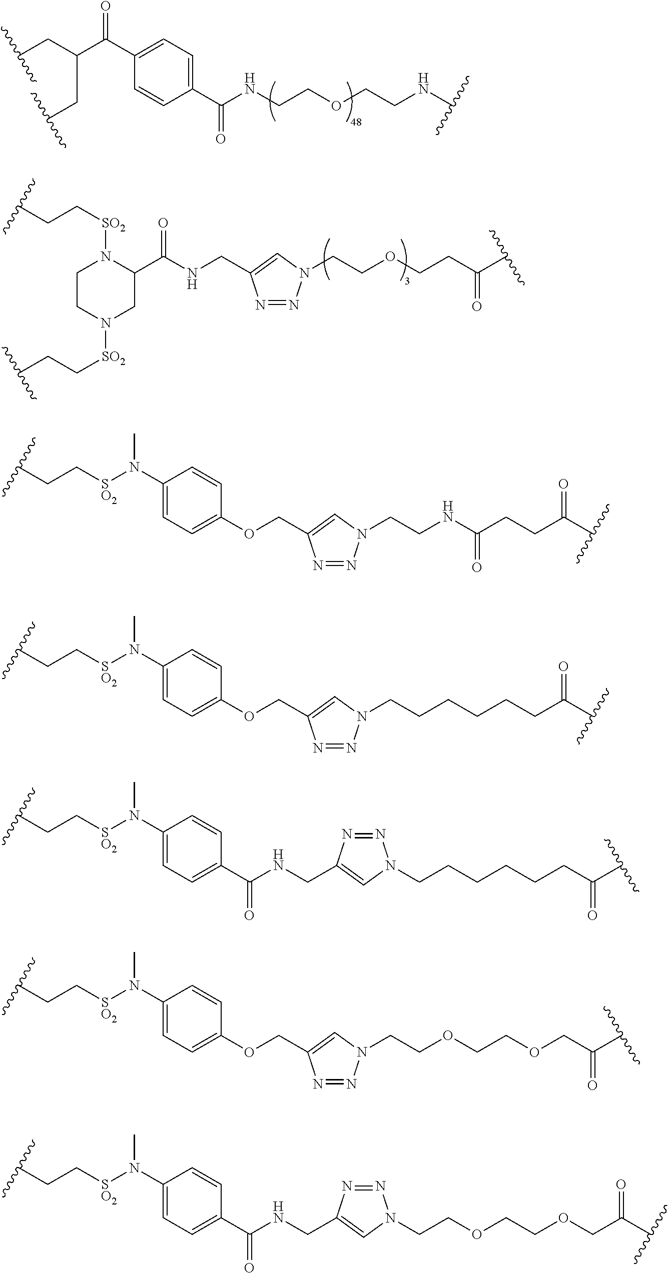

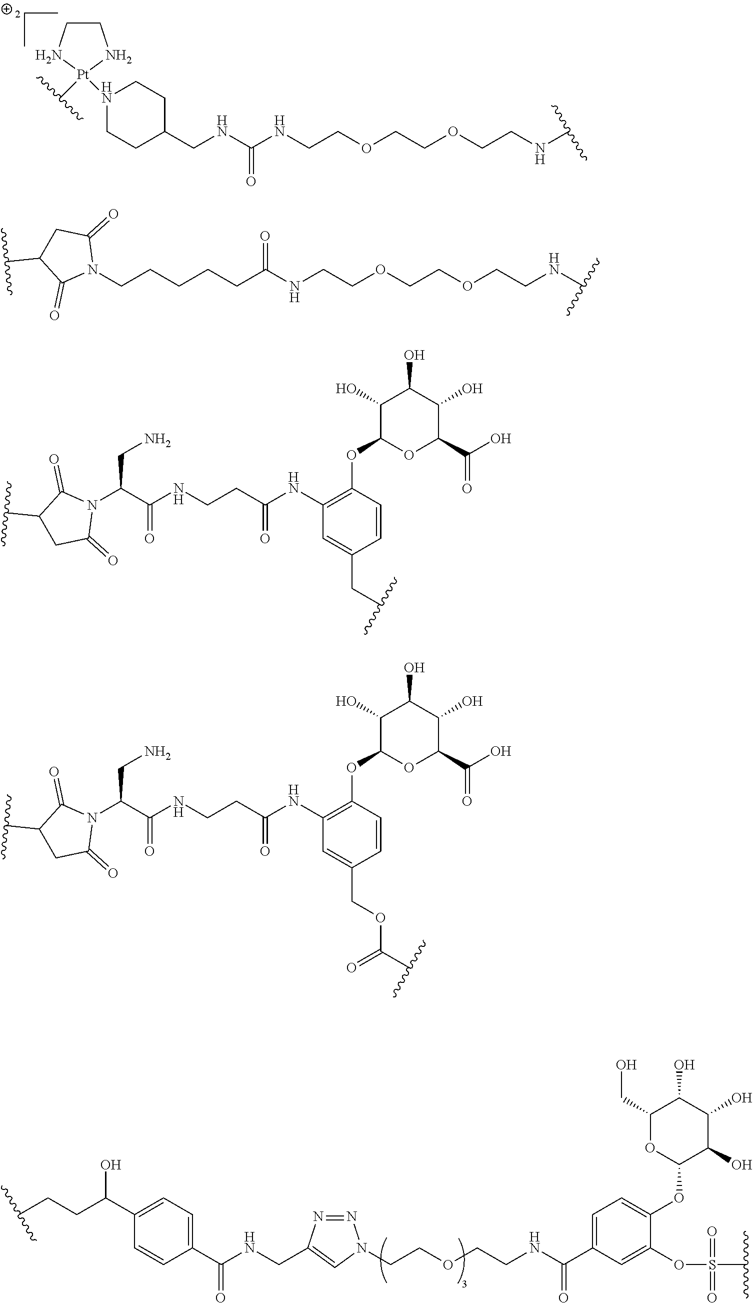

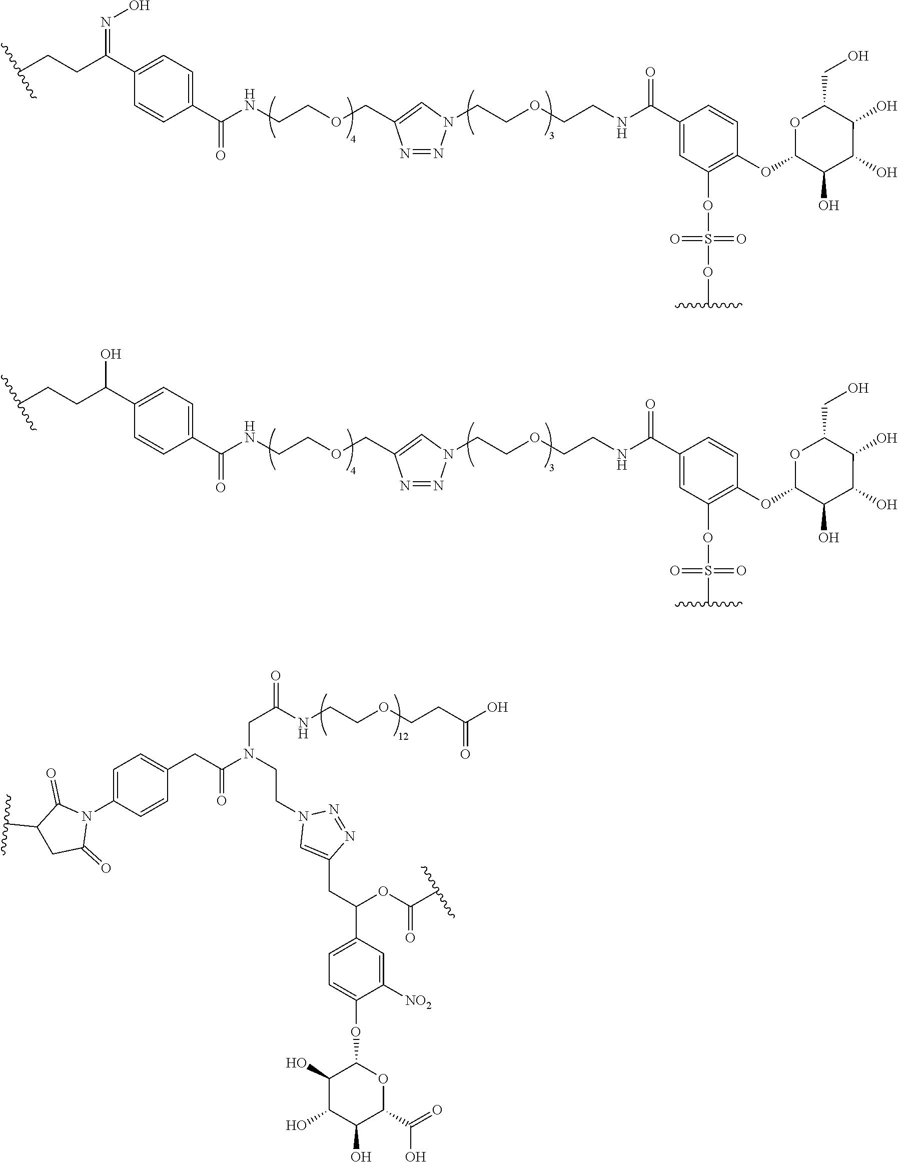

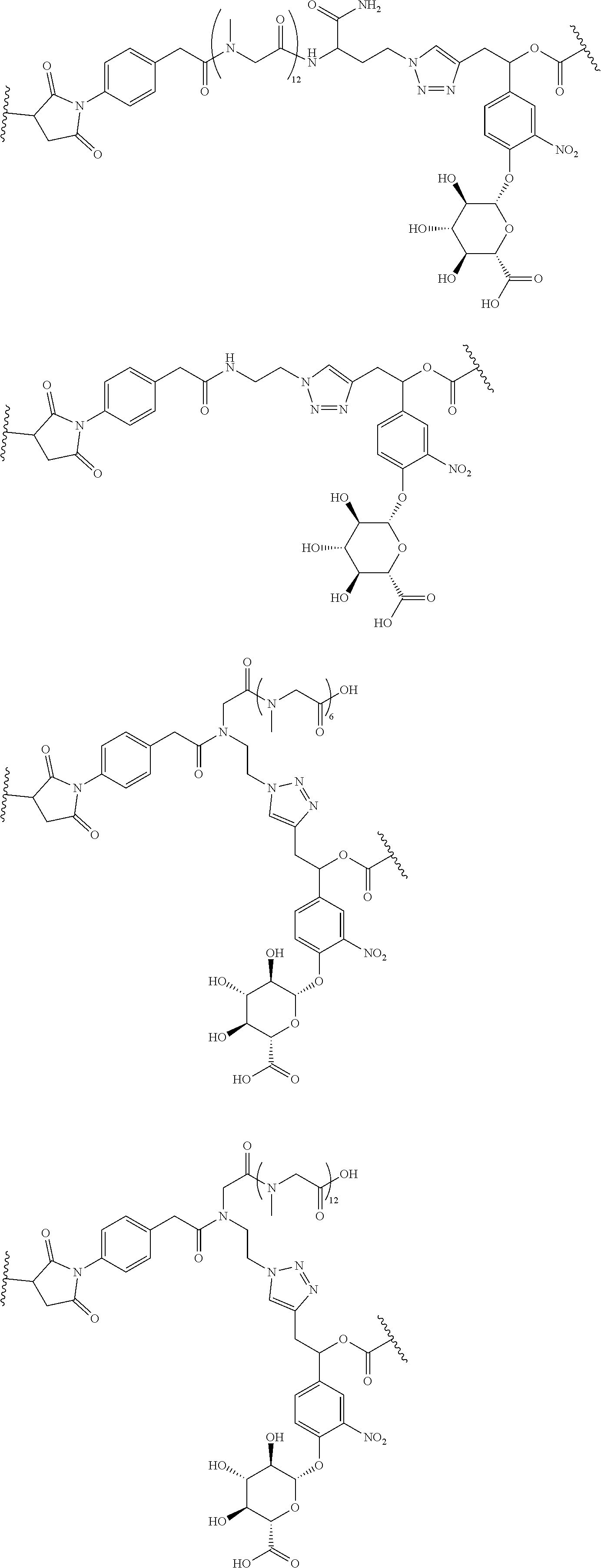

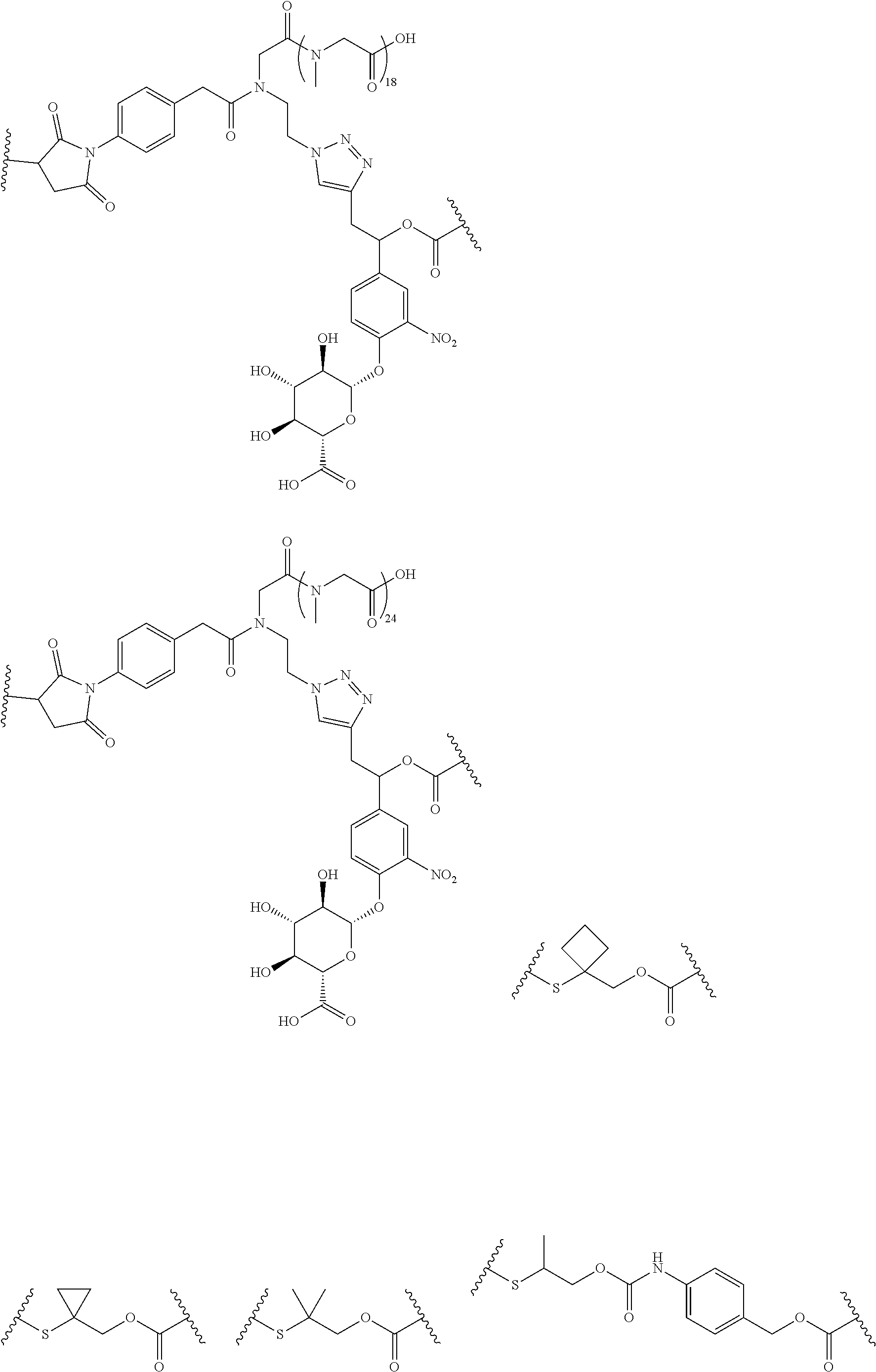

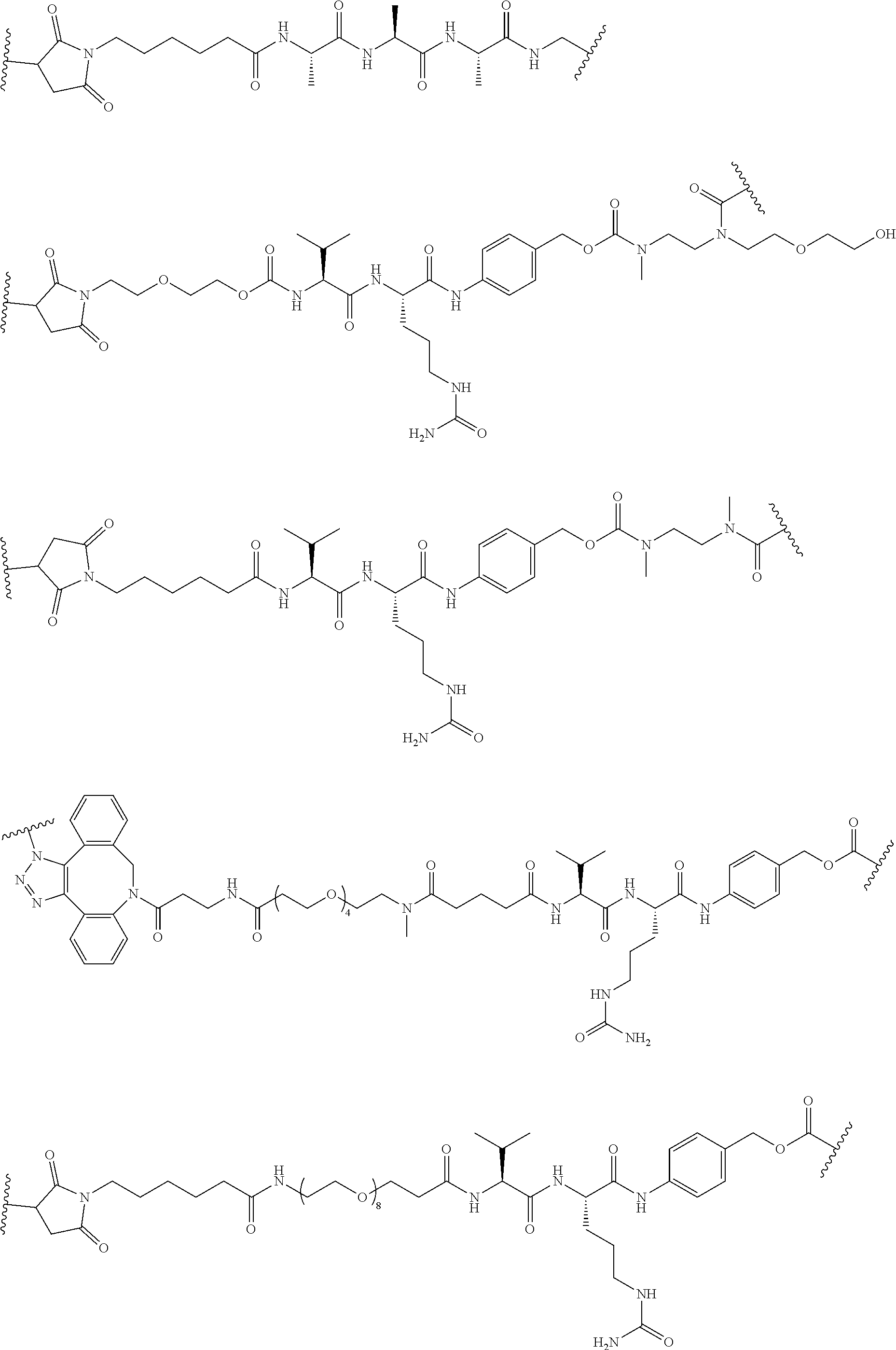

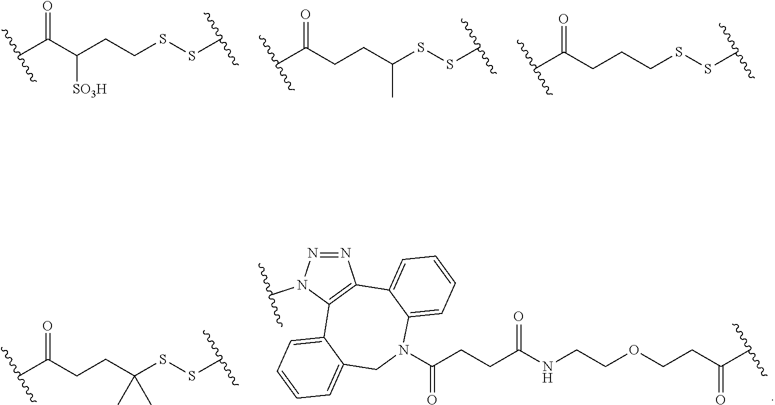

62. The conjugate of any one of claims 1 to 16, wherein each amino acid moiety of —X_{AA}— is independently selected from alanine (Ala), arginine (Arg), glycine (Gly), histidine (His), isoleucine (Ile), leucine (Leu), lysine (Lys), phenylalanine (Phe), tryptophan (Trp), tyrosine (Tyr), valine (Val), citrulline (Cit), and homocitrulline (HoCit).

63. The conjugate of any one of claims 1 to 16, wherein each amino acid moiety of —X_{AA}— is independently selected from alanine (Ala), glycine (Gly), lysine (Lys), phenylalanine (Phe), valine (Val), and citrulline (Cit).

64. The conjugate of any one of claims 1 to 15, wherein the amino acid sequence —X_{AA}— is selected from -Val-Cit-, -Cit-Val-, -Val-Ala-, -Ala-Val-, -Phe-Lys-, -Lys-Phe-, -Ala-Ala-, -Val-Val-, -Gly-Gly-, -Ala-Ala-Ala-, -Gly-Gly-Gly-, -Gly-Gly-Phe-Gly-(SEQ ID NO: 1032), -Gly-Phe-Gly-Gly-(SEQ ID NO:1033), -Gly-Gly-Gly-Phe-(SEQ ID NO:1034), -Phe-Gly-Gly-Gly-(SEQ ID NO:1035), and -Gly-Gly-Gly-Gly-(SEQ ID NO:1036).

65. The conjugate of any one of claims 1 to 16, wherein the amino acid sequence —X_{AA}— is selected from -Val-Cit-, —Cit-Val-, -Val-Ala-, -Ala-Val-, -Phe-Lys-, -Lys-Phe-, -Ala-Ala-, -Val-Val-, -Gly-Gly-, -Ala-Ala-Ala-, and -Gly-Gly-Gly-.

66. The conjugate of any one of claims 1 to 64, wherein the oligonucleotide moiety P is selected from a small interfering RNA (siRNA) molecule and an antisense oligonucleotide (ASO) molecule.

67. The conjugate of any one of claims 1 to 66, wherein the oligonucleotide moiety P is a small interfering RNA (siRNA) molecule.

68. The conjugate of any one of claims 1 to 66, wherein the oligonucleotide moiety P is an antisense oligonucleotide (ASO) molecule.

69. The conjugate of claim 66 or claim 67, wherein the siRNA molecule targets cMyc mRNA.

70. The conjugate of claim 69, wherein the siRNA molecule comprises a nucleotide sequence selected from the group consisting of 5′-GCUUUUUUGCCCUGCGUGACCAGAT-3′ (SEQ ID NO: 138), 5′-GCCACAGCAUACAUCCUGUUU-3′ (SEQ ID NO:139), and 5′-GCCACAGCAUACAUCCUGUUU-3′ (SEQ ID NO:140), 3′-CUCGAAAAAACGGGACGCACUGGUCUA-5′ (SEQ ID NO:141), 3′-UUCGGUGUCGUAUGUAGGACA-5′ (SEQ ID NO:142), and 3′-UUCGGUGUCGUAUGUAGGACA-5′ (SEQ ID NO:143).

71. The conjugate of claim 69 or claim 70, wherein the siRNA molecule comprises at least one 2′ modified nucleotide comprising a 2′-O-methyl, 2′-O-methoxyethyl(2′-O-MOE), 2′-O-aminopropyl, 2′-deoxy, 2′-deoxy-2′-fluoro, 2′-O-aminopropyl(2′-O-AP), 2′-O-dimethylaminoethyl(2′-O-DMAOE), 2′-O-dimethylaminopropyl(2′-O-DMAP), 2′-O-dimethylaminoethyloxyethyl(2′-O-DMAEOE), or 2′—O—N-methylacetamido (2′-O-NMA) modified nucleotide, at least one modified internucleotide linkage, or at least one inverted abasic moiety, or a combination thereof.

72. The conjugate of any one of claims 69 to 71, wherein the siRNA molecules comprises a nucleotide sequence selected from the group consisting of: 5′-N6-G-mC-U-U-mU-U-mU-U-G-C-mC-mC-U-mG-C-G-mU-mG-A-C-C-A-G-dA-dT-3′ (SEQ ID NO:144), 5′-N6-G-C-C-A-C-A-G-C-A-U-A-C-A-U-C-C-U-G-U-U-U-3′ (SEQ ID NO:145), 5′-N6*2′FG*mC*2′FC-mA-2′FC-mA-2′FG-mC-2′FA-mU-2′FA-mC-2′FA-mU-2′FC-mC-2′FU-mG-2′FU-mU-2′FU-3′ (SEQ ID NO: 146), 3′-mC-mU-mC-G-mA-A-A-A-A-A-C-G-G-G-mA-C-mG-C-A-C-U-G-G-mU-mC-mU-mA 5′ (SEQ ID NO:147), 3′-U-U-C-G-G-U-G-U-C-G-U-A-U-G-U-A-G-G-A-C-A-5′ (SEQ ID NO: 148), and 3′-mU*2′FU*mC-2′FG-mG-2′FU-mG-2′FU-mC-2′FG-mU-2′FA-mU-2′FG-mU-2′FA-mG-2′FG-mA*2′FC*mA-5′ (SEQ ID NO:149).

73. The conjugate of any one of claims 69 to 71, wherein the siRNA molecule hybridizes to at least 8 contiguous bases of the c-Myc mRNA.

74. The conjugate of any one of claims 69 to 73, wherein the siRNA molecule is from 8 to 50 nucleotides in length or from 10 to 30 nucleotides in length.

75. The conjugate of claim 66 or claim 67, wherein the siRNA molecule comprises a nucleotide sequence selected from:

| Strand | Sequence | SEQ ID NO |
|---|---|---|
| sense | GUAACCAAGAGUAUUCCAUDTDT | 152 |
| antisense | AUGGAAUACUCUUGGUUACDTDT | 153 |
| sense | UCUUGGUUACAUGAAAUCCCAUC | 154 |
| antisense | UGGGAUUUCAUGUAACCAAGA | 155 |
| sense | CAGAAAGAGUGUCUCAUCUUA | 156 |
| antisense | AAUGAUGAGACACUCUUUCUGGU | 157 |
| sense | UAUAUUUCCAGGAUGAAAGUCCA | 158 |
| antisense | GACUUUCAUCCUGGAAAUAUA | 159 |
| sense | ACAAAAGCAAAACAGGUCUAGAA | 160 |
| antisense | CUAGACCUGUTUUGCUUUUGUN | 161 |
| sense | AUGUUGUCCUUUUUAUCUGAGCAGC | 162 |
| | CGAAAGGCUGC | |
| antisense | UCAGAUAAAAAGGACAACAUGG | 163 |
| sense | GGUUAACACCAUUUACUUCAA | 164 |
| antisense | UUGAAGUAAAUGGUGUUAACCAG | 165 |
| sense | GAGAAUAUUUCACCCUUCA | 166 |
| antisense | UGAAGGGUGAAAUAUUCUC | 167 |
| sense | AGGAGUUCCACAUUCUGGC | 168 |
| antisense | GCCAGAAUGUGGAACUCCU | 169 |
| sense | UGAAGUAGAAGAUGCGCUU | 170 |
| antisense | AAGCGCAUCUUCUACUUCA | 171 |
| sense | AAACCCUUUGUCUUCUUAAAGCAGC | 172 |
| | CGAAAGGCUGC | |
| antisense | UUUAAGAAGACAAAGGGUUUGG | 173 |
| sense | CUGGAUCACAUGCACAAUGTT | 174 |
| antisense | CAUUGUGCAUGUGAUCCAGTT | 175 |
| sense | AAGCAAGAUAUUUUUAUAAUAN | 176 |
| antisense | UAUUAUAAAAAUAUCUUGCUUUUTT | 177 |

76. The conjugate of claim 66 or claim 68, wherein the ASO molecule targets a c-Myc gene.

77. The conjugate of claim 76, wherein the ASO molecule targets exon 2 of the c-Myc gene.

78. The conjugate of claim 76 or claim 77, wherein the ASO molecule comprises a sequence of UUCACCATCTCUC (SEQ ID NO:150).

79. The conjugate of any one of claims 76-78, wherein the ASO molecule comprises a sequence of mU*mU*mC*A*C*C*A*T*C*T*C*mU*mC (SEQ ID NO:151), wherein m denotes 2′-O-methyl-modified ribose nucleotides and * denotes phosphorothioate linkage.

80. The conjugate of claim 66 or 66, wherein the ASO molecule comprises a sequence selected from Table 7 and Table 8.

81. The conjugate of any one of claims 1 to 80, wherein the VL CDR1 comprises the amino acid sequence of SEQ ID NO:9, CDR2 comprises the amino acid sequence of SEQ ID NO:10, CDR3 comprises SEQ ID NO:11; and the VH CDR1 comprises the amino acid sequence of SEQ ID NO: 15, CDR2 comprises the amino acid sequence of SEQ ID NO:4, CDR3 comprises the amino acid sequence of SEQ ID NO:5.

82. The conjugate of any one of claims 1 to 80, wherein the VL CDR1 comprises the amino acid sequence of SEQ ID NO:29, CDR2 comprises the amino acid sequence of SEQ ID NO:10, CDR3 comprises SEQ ID NO:11; and the VH CDR1 comprises the amino acid sequence of SEQ ID NO: 15, CDR2 comprises the amino acid sequence of SEQ ID NO:26, CDR3 comprises the amino acid sequence of SEQ ID NO:5.

83. The conjugate of any one of claims 1 to 80, wherein the antibody or antigen-binding fragment thereof comprises a light chain variable region (VL) comprising an amino acid sequence of SEQ ID NO:21 and a heavy chain variable region (VH) comprising an amino acid sequence of SEQ ID NO:14.

84. The conjugate of any one of claims 1 to 80, wherein the antibody or antigen-binding fragment thereof a full-length light chain (LC) comprising an amino acid sequence of SEQ ID NO: 20 and a full-length heavy chain (HC) comprising an amino acid sequence of SEQ ID NO:13.

85. The conjugate of any one of claims 1 to 84, wherein the antibody or antigen-binding fragment thereof comprises: a light chain variable domain (VL) comprising an amino acid sequence that is at least 95% identical to an amino acid sequence selected from the group consisting of 3E10-VL-H₁ (SEQ ID NO:85), 3E10-VL-H₂ (SEQ ID NO:86), 3E10-VL-H₃ (SEQ ID NO:87), 3E10-VL-H₄ (SEQ ID NO:88), 3E10-VL-H₅ (SEQ ID NO:89), and 3E10-VL-H₆ (SEQ ID NO:90); and a heavy chain variable domain (VH) comprising an amino acid sequence that is at least 95% identical to an amino acid sequence selected from the group consisting of 3E10-VH-H₁ (SEQ ID NO: 64), 3E10-VH-H₂ (SEQ ID NO:65), 3E10-VH-H₃ (SEQ ID NO:66), 3E10-VH-H₄ (SEQ ID NO: 67), 3E10-VH-H₅ (SEQ ID NO:68), 3E10-VH-H₆ (SEQ ID NO:69), and 3E10-VH-H₇ (SEQ ID NO: 70).

86. The conjugate of any one of claims 1 to 85, wherein the antibody or antigen-binding fragment thereof comprises: a light chain variable domain (VL) comprising an amino acid sequence selected from the group consisting of 3E10-VL-H₁ (SEQ ID NO:85), 3E10-VL-H₂ (SEQ ID NO: 86), 3E10-VL-H₃ (SEQ ID NO:87), 3E10-VL-H₄ (SEQ ID NO:88), 3E10-VL-H₅ (SEQ ID NO: 89), and 3E10-VL-H₆ (SEQ ID NO:90); and a heavy chain variable domain (VH) comprising an amino acid sequence selected from the group consisting of 3E10-VH-H₁ (SEQ ID NO:64), 3E10-VH-H₂ (SEQ ID NO:65), 3E10-VH-H₃ (SEQ ID NO:66), 3E10-VH-H₄ (SEQ ID NO:67), 3E10-VH-H₅ (SEQ ID NO:68), 3E10-VH-H₆ (SEQ ID NO:69), and 3E10-VH-H₇ (SEQ ID NO: 70).

87. The conjugate of any one of claims 1 to 86, wherein the antibody or antigen-binding fragment thereof comprises a VL/VH pair selected from the group consisting of (a) VL1 (SEQ ID NO: 85) and VH1 (SEQ ID NO:64), (b) VL1 (SEQ ID NO:85) and VH2 (SEQ ID NO:65), (c) VL1 (SEQ ID NO:85) and VH3 (SEQ ID NO:66), (d) VL1 (SEQ ID NO:85) and VH4 (SEQ ID NO: 67), (e) VL2 (SEQ ID NO:86) and VH1 (SEQ ID NO:64), (f) VL2 (SEQ ID NO:86) and VH2 (SEQ ID NO:65), (g) VL2 (SEQ ID NO:86) and VH3 (SEQ ID NO:66), (h) VL2 (SEQ ID NO:86) and VH4 (SEQ ID NO:67), (i) VL3 (SEQ ID NO:87) and VH1 (SEQ ID NO:64), (j) VL3 (SEQ ID NO: 87) and VH2 (SEQ ID NO:65), (k) VL3 (SEQ ID NO:87) and VH3 (SEQ ID NO:66), (1) VL3 (SEQ ID NO:87) and VH4 (SEQ ID NO:67), (m) VL4 (SEQ ID NO:88) and VH1 (SEQ ID NO: 64), (n) VL4 (SEQ ID NO:88) and VH2 (SEQ ID NO:65), (0) VL4 (SEQ ID NO:88) and VH3 (SEQ ID NO:66), (p) VL4 (SEQ ID NO:88) and VH4 (SEQ ID NO:67), (q) VL5 (SEQ ID NO:89) and VH5 (SEQ ID NO:68), (r) VL5 (SEQ ID NO:89) and VH6 (SEQ ID NO:69), (s) VL6 (SEQ ID NO: 90) and VH5 (SEQ ID NO:68), and (t) VL6 (SEQ ID NO:90) and VH6 (SEQ ID NO:69).

88. The conjugate of any one of claims 1 to 87, wherein the antibody or antigen-binding fragment thereof comprises: a light chain variable domain (VL) comprising 3E10-VL-H₆ (SEQ ID NO: 90) and a heavy chain variable domain (VH) comprising 3E10-VH-H₆ (SEQ ID NO:69).

89. The conjugate of any one of claims 1 to 88, wherein the antibody or antigen-binding fragment thereof comprises: a light chain variable domain (VL) comprising the amino acid sequence (DIQMTQSPSSLSASLGDRATITCRASKTVSTSSYSYMHWYQQKPGQPPKLLIKYASYLE SGVPSRFSGSGSGTDFTLTISSLQPEDAATYYCQHSREFPWTFGGGTKVEIK) (SEQ ID NO: 117) and a heavy chain variable domain (VH) comprising the amino acid sequence (EVQLVESGGGLVQPGGSLRLSCAASGFTFSNYGMHWVRQAPGKGLEWVSYISSGSSTI YYADSVKGRFTISRDNAKNSLYLQMNSLRAEDTAVYYCARRGLLLDYWGQGTTVTVS S) (SEQ ID NO:105).

90. A method for treating a subject in need thereof, the method comprising administering a therapeutically effective amount of a conjugate according to any one of claims 1 to 89 to the subject.

91. A method for treating cancer in a subject in need thereof, the method comprising administering to the subject a therapeutically effective amount of a conjugate according to any one of claims 1 to 89 to the subject.

92. The method of claim 91, wherein the cancer is a carcinoma, a sarcoma, a blastoma, a papilloma, or an adenoma.

93. The method of claim 91, wherein the cancer is metastatic cancer.

94. The method of any one of claims 91-93, wherein the cancer is selected from the group consisting of bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, melanoma, myeloma, ovarian cancer, pancreatic cancer, prostate cancer, renal cancer, thyroid cancer, and uterine cancer.

95. The method of claim 94, wherein the cancer is a skin cancer selected from the group consisting of basal cell carcinoma, squamous cell carcinoma, and melanoma.

96. The method of claim 94, wherein the cancer is melanoma.

97. The method of claim 91, wherein the cancer is ovarian cancer.

98. The method of claim 91, wherein the cancer is colorectal cancer.

99. The method of claim 91, wherein the cancer is breast cancer.

100. The method of claim 91, wherein the cancer is BRCA2-breast cancer.

101. The method of claim 91, wherein the cancer is triple negative breast cancer.

102. The method of claim 91, wherein the cancer is lung cancer.

103. The method of claim 91, wherein the cancer is non-small cell lung cancer (NSCLC).

104. The method of any one of claims 91-93, wherein the cancer is a cancer of the central nervous system.

105. The method of claim 91, wherein the cancer is a neuroepithelial brain or spinal tumor selected from the group consisting of a medulloblastoma, an astrocytic tumor, an oligodendroglial tumor, an oligoastrocytic tumor, an ependymal tumor, a choroid plexus tumor, a neuronal or mixed neuronal-glial tumor, a tumor of the pineal region, an embryonal tumor, or an otherwise uncategorized neuroepithelial tumor.

106. The method of any one of claims 91-105, wherein the cancer expresses a functional ENT2 transporter.

107. A conjugate according to any one of claims 1 to 89 for use in the treatment of cancer.

108. The conjugate of claim 107, wherein the cancer is a carcinoma, a sarcoma, a blastoma, a papilloma, or an adenoma.

109. The conjugate of claim 107, wherein the cancer is metastatic cancer.

110. The conjugate of any one of claims 107-109, wherein the cancer is selected from the group consisting of bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, melanoma, myeloma, ovarian cancer, pancreatic cancer, prostate cancer, renal cancer, thyroid cancer, and uterine cancer.

111. The conjugate of claim 110, wherein the cancer is a skin cancer selected from the group consisting of basal cell carcinoma, squamous cell carcinoma, and melanoma.

112. The conjugate of claim 110, wherein the cancer is melanoma.

113. The conjugate of claim 107, wherein the cancer is ovarian cancer.

114. The conjugate of claim 107, wherein the cancer is colorectal cancer.

115. The conjugate of claim 107, wherein the cancer is breast cancer.

116. The conjugate of claim 107, wherein the cancer is BRCA2-breast cancer.

117. The conjugate of claim 107, wherein the cancer is triple negative breast cancer.

118. The conjugate of claim 107, wherein the cancer is lung cancer.

119. The conjugate of claim 107, wherein the cancer is non-small cell lung cancer (NSCLC).

120. The conjugate of any one of claims 107-109, wherein the cancer is a cancer of the central nervous system.

121. The conjugate of claim 107, wherein the cancer is a neuroepithelial brain or spinal tumor selected from the group consisting of a medulloblastoma, an astrocytic tumor, an oligodendroglial tumor, an oligoastrocytic tumor, an ependymal tumor, a choroid plexus tumor, a neuronal or mixed neuronal-glial tumor, a tumor of the pineal region, an embryonal tumor, or an otherwise uncategorized neuroepithelial tumor.

122. The conjugate of any one of claims 107-121, wherein the cancer expresses a functional ENT2 transporter.

123. The method of any one of claims 91-105 or conjugate of any one of claims 107-122, wherein the administering is by parenteral administration.

124. The method of claim 123, wherein the parenteral administration is intramuscular administration, intravenous administration, or subcutaneous administration.
